(19) United States
(12) Patent Application Publication
(10) Pub. No.: US 2019/0381160 A1
(43) Pub. Date: Dec. 19, 2019

(54) PERSONALIZED DELIVERY VECTOR-BASED IMMUNOTHERAPY AND USES THEREOF

(71) Applicant: ADVAXIS, INC., Princeton, NJ (US)

(72) Inventors: Robert PETIT, Newtown, PA (US); Kyle PERRY, Lawrenceville, NJ (US); Michael F. PRINCIOTTA, Hightstown, NJ (US); Daniel J O'CONNOR, Pennington, NJ (US)

(73) Assignee: Advaxis, Inc., Princeton, NJ (US)

(21) Appl. No.: 15/576,178

(22) PCT Filed: May 26, 2016
(86) PCT No.: PCT/US2016/034301

§ 371 Date: Nov. 21, 2017

Related U.S. Application Data
(60) Provisional application No. 62/218,936, filed on Sep. 15, 2015.
(60) Provisional application No. 62/174,692, filed on Jun. 12, 2015.
(60) Provisional application No. 62/166,591, filed on May 26, 2015.

(51) Int. Cl. A61K 39/02; C12Q 1/6886; C12N 15/74; C12N 15/62; C12N 1/20; C07K 14/195; A61K 39/00; C12N 7/00; A61P 35/00

(52) U.S. Cl. CPC A61K 39/02; C12Q 1/6886; C12N 15/74; C12N 15/62; C12N 1/20; C07K 14/195; C12N 2710/20034; C12N 7/00; A61P 35/00; A61K 2039/523; A61K 2039/522; C07K 2319/00; A61K 2039/585; A61K 39/001106

(57) ABSTRACT

This invention provides a system of providing and creating personalized immunotherapeutic compositions for a subject having a disease or condition, including therapeutic immunotherapy delivery vectors and methods of making the same comprising gene expression constructs expressing peptides associated with one or more neo-epitopes or peptides containing mutations that are specific to a subject's cancer or unhealthy tissue. A delivery vector of this invention includes bacterial vectors including Listeria bacterial vectors; or viral vectors, peptide immunotherapy vectors; or DNA immunotherapy vectors, comprising one or more fusion proteins comprising one or more peptides comprising one or more neo-epitopes present in disease-bearing biological samples obtained from the subject. This invention also provides methods of using the same for inducing an immune response against a disease or condition, including a tumor or cancer, or an infection, or an autoimmune disease or an organ transplant rejection in the subject.

### CROSS REFERENCE TO RELATED APPLICATIONS

This application claims the benefit of U.S. Application No. 62/166,591, filed May 26, 2015, U.S. Application No. 62/174,692, filed Jun. 12, 2015, and U.S. Application No. 62/218,936, filed, Sep. 15, 2015, each of which is herein incorporated by reference in its entirety for all purposes.

### REFERENCE TO A SEQUENCE LISTING SUBMITTED AS A TEXT FILE VIA EFS WEB

The Sequence Listing written in file SEQLIST_ST25.txt is 488 kb, was created on May 25, 2016, and is hereby incorporated by reference.

### FIELD OF INTEREST

This invention provides a personalized immunotherapy composition for a subject having a disease or condition, including therapeutic immunotherapy delivery vectors and methods of making the same comprising gene expression constructs expressing peptides associated with one or more neo-epitopes or peptides containing mutations that are specific to a subject's cancer or unhealthy tissue. A delivery vector of this invention includes bacterial vectors; or viral vectors, or peptide immunotherapy vectors; or DNA immunotherapy vectors including Listeria bacterial vectors comprising one or more fusion proteins comprising one or more peptides comprising one or more neo-epitopes present in disease-bearing biological samples obtained from the subject. This invention also provides methods of using the same for inducing an immune response against a disease or condition, including a tumor or cancer, or an infection, or an autoimmune disease or an organ transplant rejection in the subject.

### BACKGROUND OF THE INVENTION

Before personalized medicine, most patients with a specific type and stage of cancer received the same treatment. However, it has become clear to doctors and patients that some treatments worked well for certain patients and not as well for others. Thus, there is a need to develop effective, personalized immunotherapies effective for a particular tumor. Personalized treatment strategies may be more effective for an individual and cause fewer side effects than would be expected with standard treatments.

Tumors develop due to mutations in a person's DNA, which can cause the production of mutated or abnormal proteins, comprising potential neo-epitopes not present within the corresponding normal protein produced by the host. Some of these neo-epitopes may stimulate T-cell responses and mediate the destruction of early-stage cancerous cells by the immune system so that clinical evidence of a cancer does not develop. In cases of established cancer, however, the immune response has been insufficient. A large body of data has been generated regarding the development of therapeutic immunotherapies that target natural sequence tumor-associated, over-expressed or inappropriately expressed biomarkers in cancer. However demonstration of clear clinical benefit associated with these treatments has proven quite difficult with only one therapeutic immunotherapy being approved by the FDA at the time of this writing. A major reason for this is that as part of central tolerance that develops in all individuals, any T cells that have high binding affinity toward natural sequence peptides are identified as self-antigens and these self-reactive clones are eliminated by the thymus early in life, or otherwise inactivated through mechanisms of tolerance to prevent auto-immunity.

Neo-epitopes are potentially immunogenic epitopes present within a protein associated with a disease that result from a change in the DNA that occurs later in life, such as an acquired mutation or genomic change caused by changes in the DNA of certain cells. For example a cancer, wherein the specific “neo-epitope” is not present within the corresponding normal protein associated with cells (in the same individual) that do not harbor the acquired DNA abnormality which results in the neoepitope expressed in a subjects cells that are not diseased or comprising a disease-bearing tissue therein. Neo-epitopes may be challenging to identify, however doing so and developing treatments that target them would be advantageous for use within a personalized treatment strategy. The specific acquired DNA abnormality(s) are very individual to both the specific patient's diseased cells as well as the particular epitope that their immune system might recognize. Because these factors vary from person to person, a personalized approach must be employed to target the multiple neoepitopes, which may number in the thousands, that occur in a person with disease like a cancer or pre-malignant condition.

Listeria monocytogenes (Lm) is a Gram-positive facultative intracellular pathogen that can cause listeriosis. In its intercellular lifecycle, Lm enters host cells through by phagocytosis or by active invasion of non-phagocytic cells. Following internalization, Lm may mediate its escape from the membrane bound phagosome/vacuole by secretion of several bacterial virulence factors, primarily the pore-forming protein listeriolysin O (LLO), enabling the bacteria to enter the host cell cytoplasm. In the cytoplasm Lm replicates and spreads to adjacent cells based on the mobility facilitated by the bacterial actin-polymerizing protein (ActA) along with other virulence factors. In the cytoplasm, Lm-secreted proteins and ultimately Lm structural proteins are degraded by the proteasome and processed into peptides that can associate with MHC class I molecules in the endoplasmic reticulum. These MHC-peptide complexes are transported to the cell surface and can be presented to and recognized by target-specific T cells. This unique characteristic makes it a very attractive T cell generating vector in that tumor antigen can be presented with MHC class I molecules to activate tumor-specific cytotoxic T lymphocytes (CTLs). CTLs are the primary target-specific effector cells that kill other cells in the body like cancer cells or cells that harbor an intracellular infection.

In addition, once internalized, Lm is also processed in the phagolysosomal compartment and its peptides presented on MHC Class II which can generate antigen specific CD4-T cell responses which can assits CTLs in target-directed killing of cabcerous or infected cells.

In addition, since the vector is a live bacteria its composition can stimulate a number of triggers of innate immunity which includes several external, intercellular, and cytosolic molecular pattern receptors, including PAMPs, DAMPS, and TLR's. For example, recognition of peptidoglycan by nuclear oligomerization domain-like receptors and Lm DNA by DNA sensors, AIM2 and STING, and activate inflammatory and immune-modulatory cascades. This combination of inflammatory responses and efficient delivery of antigens to the MHC I and MHC II pathways makes Lm a powerful immunotherapy vector in treating, protecting against, and inducing an immune response against a tumor.

Targeting neo-epitopes specific to a subject's cancer as a component of a Listeria based immunotherapy vector that additionally stimulates T-cell response and can also be used in combination with other therapies, may provide an immunotherapy that is both personalized to a subject's cancer and effective in the treatment of the cancer. The fusion of a highly immunogenic peptide antigen to a targeted peptide can significantly increase the immunogenicity of the target antigen or the ability of immunotherapies to stimulate T cells that have escaped tolerance mechanisms, may have a particular potential as immunotherapies.

The present invention provides personalized immunotherapy compositions and uses thereof for targeting potential neo-epitopes within abnormal or unhealthy tissue of a subject, wherein the immunotherapy comprises the use of a recombinant Listeria immunotherapy as a delivery and immunotherapeutic vector for expressing peptides and/or fusion polypeptides comprising said neo-epitopes in order to enhance an immune response targeting these neo-epitopes. The personalized immunotherapies created may effectively treat, prevent, prolong life, or reduce the incidence of a disease, for example cancer in a subject. Further, recombinant Listeria of the present invention may effectively be used in combination with other anti-disease or anti-cancer therapies.

### SUMMARY OF THE INVENTION

In one aspect, the present invention relates to a system for providing a personalized immunotherapy system created for a subject having a disease or condition, said system comprising:
a. an attenuated Listeria strain delivery vector; and
b. a plasmid vector for transforming said Listeria strain, said plasmid vector comprising a nucleic acid construct comprising one or more open reading frames encoding one or more peptides comprising one or more neo-epitopes or potential neo-epitopes, wherein said neo-epitope(s) comprise immunogenic epitopes present in a disease-bearing tissue or cell of said subject having said disease or condition;
   wherein transforming said Listeria strain with said vector creates a personalized immunotherapy system targeted to said subject's disease or condition.

In one aspect, the present invention relates to a system for providing a personalized immunotherapy system created for a subject having a disease or condition, said system comprising:
a. a delivery vector; and optionally
b. a plasmid vector for transforming said delivery vector, said plasmid vector comprising a nucleic acid construct comprising one or more open reading frames encoding one or more peptides comprising one or more neo-epitopes, wherein said neo-epitope(s) comprise immunogenic epitopes present in a disease-bearing tissue or cell of said subject having said disease or condition.

In a related aspect, said delivery vector comprises a bacterial delivery vector. In another related aspect said delivery vector comprises a viral vector delivery vector. In another related aspect said delivery vector comprises a peptide immunotherapy delivery vector. In another related aspect, said delivery vector comprises a DNA plasmid immunotherapy delivery vector.

In a related aspect, the disease or condition comprises an infectious disease, an autoimmune disease, an organ rejection of a transplant, or a tumor, or cancer, or dysplastic cells or tissue. In another aspect, the adaptive immune response is facilitated and enhanced by an innate immune response triggered by the administration of the live, attenuated immunotherapy agents to a person as treatment. In another related aspect, the immune response is an adaptive immune response. In yet another related aspect, the immune response is a T-cell immune response. In another related aspect, an attenuated, recombinant Listeria is cultivated, cryopreserved, optionally lyophilized and spray-dried, and administered as a form of treatment to the subject either alone or in combination with other potentially beneficial treatments for their disease. The treatment can include repeated administrations.

In another aspect, the present invention relates to a process for creating a personalized immunotherapy for a subject having a disease or condition, the process comprising the steps of:
a. comparing one or more open reading frames (ORFs) in nucleic acid sequences extracted from a disease-bearing biological sample with one or more ORFs in nucleic acid sequences extracted from a healthy biological sample, wherein said comparing identifies one or more neo-epitopes encoded within said one or more ORFs from the disease-bearing sample;
b. screening peptides comprising said one or more neo-epitopes for an immunogenic response;
c. transforming an attenuated Listeria strain with a vector comprising a nucleic acid sequence that encodes a one or more peptides comprising said one or more immunogenic neo-epitopes;
d. and, alternatively storing said attenuated recombinant Listeria for administering to said subject at a pre-determined period or administering said attenuated recombinant Listeria strain to said subject, wherein said attenuated recombinant Listeria strain is administered as part of an immunogenic composition.

In a related aspect, the invention relates to a recombinant attenuated Listeria strain comprising the following:
a. a nucleic acid molecule, said nucleic acid molecule comprising a first open reading frame encoding a fusion polypeptide, wherein said fusion polypeptide comprises an immunogenic polypeptide or fragment thereof fused to one or more peptides comprising one or more neo-epitopes provided herein; or,
b. a minigene nucleic acid construct comprising a first open reading frame encoding a chimeric protein, wherein said chimeric protein comprises:
   i. a bacterial secretion signal sequence,
   ii. a ubiquitin (Ub) protein,
   iii. one or more peptides comprising one or more neo-epitopes provided herein; and
wherein the signal sequence, the ubiquitin, and the one or more peptides in i.-iii. are operatively linked or arranged in tandem from the amino-terminus to the carboxy-terminus.

In a related aspect, the bacterial sequence is a Listerial sequence, wherein in some embodiments, said Listerial sequence is an hly signal sequence or an actA signal sequence.

In another related aspect, the present invention relates to an immunogenic composition comprising an attenuated recombinant Listeria strain provided herein, and a pharmaceutically acceptable carrier.

In another related aspect, the composition comprises one or more attenuated Listeria strains, wherein each attenuated Listeria strain expresses one or more different peptides comprising one or more neo-epitopes. In another aspect, each attenuated Listeria expresses a range of neo-epitopes.

In a related aspect, the process provided herein allows the generation of a personalized enhanced anti-disease, or anti-infectious disease, anti-autoimmune disease, anti-rejection of an organ transplant, or anti-tumor or anticancer immune response in said subject having a disease or condition.

In another related aspect, the process provided herein allows personalized treatment or prevention of said disease, or said infection, said autoimmune disease, said rejection of an organ transplant, or said tumor or cancer in a subject.

In another related aspect, the process provided herein increases survival time in said subject having said disease or condition, or said infection, or said autoimmune disease, or said organ transplant rejection, or said tumor or cancer.

In one aspect, the present invention relates to a recombinant attenuated Listeria strain, wherein the Listeria strain comprises a nucleic acid sequence comprising one or more open reading frames encoding one or more peptides comprising one or more personalized neo-epitopes, wherein the neo-epitope(s) comprises immunogenic epitopes present in a disease or condition bearing tissue or cell of a subject having the disease or condition.

In one aspect, the present invention relates to a process for creating a personalized immunotherapy for a subject having a disease or condition, the process comprising the steps of: (a) comparing one or more open reading frames (ORFs) in nucleic acid sequences extracted from a disease-bearing biological sample with one or more ORFs in nucleic acid sequences extracted from a healthy biological sample, wherein said comparing identifies one or more nucleic acid sequences encoding one or more peptides comprising one or more neo-epitopes encoded within said one or more ORFs from the disease-bearing sample; (b) transforming an attenuated Listeria strain with a vector comprising a nucleic acid sequence encoding one or more peptides comprising said one or more neo-epitopes identified in a.; and, alternatively storing said attenuated recombinant Listeria for administering to said subject at a pre-determined period or administering a composition comprising said attenuated recombinant Listeria strain to said subject, and wherein said administering results in the generation of a personalized T-cell immune response against said disease or said condition; optionally, (c) obtaining a second biological sample from said subject comprising a T-cell clone or T-infiltrating cell from said T-cell immune response and characterizing specific peptides comprising one or more neo-epitopes bound by MHC Class I or MHC Class II molecules on said T cells, wherein said one or more neo-epitopes are immunogenic; (d) screening for and selecting a nucleic acid construct encoding one or more peptides comprising one or more immunogenic neo-epitope identified in c.; and, (e) transforming a second attenuated recombinant Listeria strain with a vector comprising a nucleic acid sequence encoding one or more peptides comprising said one or more immunogenic neo-epitopes; and, alternatively storing said second attenuated recombinant Listeria for administering to said subject at a pre-determined period or administering a second composition comprising said second attenuated recombinant Listeria strain to said subject, wherein said process creates a personalized immunotherapy for said subject.

A process for creating a personalized immunotherapy for a subject having a disease or condition, the process comprising the steps of: (a) comparing one or more open reading frames (ORFs) in nucleic acid sequences extracted from a disease-bearing biological sample with one or more ORFs in nucleic acid sequences extracted from a healthy biological sample, wherein said comparing identifies one or more nucleic acid sequences encoding one or more peptides comprising one or more neo-epitopes encoded within said one or more ORFs from the disease-bearing sample; (b) transforming a vector with a nucleic acid sequence encoding one or more peptides comprising said one or more neo-epitopes identified in a., or generating a DNA immunotherapy vector or a peptide immunotherapy vector using said nucleic acid sequence encoding one or more peptides comprising said one or more neo-epitopes identified in a.; and, alternatively storing said vector or said DNA immunotherapy or said peptide immunotherapy for administering to said subject at a pre-determined period or administering a composition comprising said vector, said DNA immunotherapy or said peptide immunotherapy to said subject, and wherein said administering results in the generation of a personalized T-cell immune response against said disease or said condition; and optionally, (c) obtaining a second biological sample from said subject comprising a T-cell clone or T-infiltrating cell from said T-cell immune response and characterizing specific peptides comprising one or more immunogenic neo-epitopes bound by MHC Class I or MHC Class II molecules on said T cells; (d) screening for and selecting a nucleic acid construct encoding one or more peptides comprising one or more immunogenic neo-epitope identified in c.; and, (e) transforming a second vector with a nucleic acid sequence comprising one or more open reading frames encoding one or more peptides comprising said one or more immunogenic neo-epitopes or generating a DNA immunotherapy vector or a peptide immunotherapy vector using said nucleic acid sequence encoding one or more peptides comprising said one or more immunogenic neo-epitopes identified in c.; and, alternatively storing said vector or said DNA immunotherapy or said peptide immunotherapy for administering to said subject at a pre-determined period, or administering a composition comprising said vector, said DNA immunotherapy or said peptide immunotherapy to said subject, wherein said process creates a personalized immunotherapy for said subject.

In one aspect, the present invention relates to a recombinant attenuated Listeria strain comprising: (a) a nucleic acid molecule, the nucleic acid molecule comprising a first open reading frame encoding a fusion polypeptide, wherein the fusion polypeptide comprises an immunogenic polypeptide or fragment thereof fused to one or more peptides comprising one or more neo-epitopes provided herein; or, (b) a minigene nucleic acid construct comprising one or more open reading frames encoding a chimeric protein, wherein the chimeric protein comprises: (i) a bacterial secretion signal sequence, (ii) a ubiquitin (Ub) protein, (iii) one or more peptides comprising one or more neo-epitopes provided herein; and, wherein the signal sequence, the ubiquitin and one or more peptides in (a)-(c) are operatively linked or arranged in tandem from the amino-terminus to the carboxy-terminus, wherein the neo-epitope(s) comprise immunogenic epitopes present in a disease or condition bearing tissue or cell of a subject having the disease or condition.

In a related aspect, administrating the Listeria strain to a subject having said disease or condition generates an immune response targeted to the subject's disease or condition.

In a related aspect, the strain is a personalized immunotherapy vector for said subject targeted to said subject's disease or condition.

In a related aspect, the neo-epitope sequences are tumor specific, metastases specific, bacterial infection specific, viral infection specific, and any combination thereof.

In a related aspect, one or more neo-epitope comprises between about 5 to 50 amino acids.

In a related aspect, the neo-epitopes are determined using exome sequencing or transcriptome sequencing of the disease-bearing tissue or cell.

In a related aspect, one or more neo-epitope(s) are screened for immunosuppressive epitopes, wherein immunosuppressive epitopes are excluded from the nucleic acid molecule.

In a related aspect, one or more neo-epitope(s) are codon optimized for expression and secretion according to the Listeria strain.

In a related aspect, one or more peptides are each fused to an immunogenic polypeptide or fragment thereof.

In a related aspect, the immunogenic polypeptide is a mutated Listeriolysin O (LLO) protein, a truncated LLO (tLLO) protein, a truncated ActA protein, an ActA-PEST2 (LA-242) fusion, or a PEST amino acid sequence.

In a related aspect, the disease or condition is an infectious disease, an autoimmune disease, or a tumor or a cancer, or dysplasia.

In a related aspect, the infectious disease comprises a viral or bacterial infection.

In a related aspect, one or more neo-epitopes comprise an infectious disease-associated-specific epitope.

In a related aspect, the attenuated Listeria comprises a mutation in one or more endogenous genes.

In a related aspect, the Listeria strain further comprises a nucleic acid construct comprising one or more open reading frames encoding one or more one or more immunomodulatory molecule(s).

In a related aspect, a personalized immunotherapy composition comprising one or more Listeria strain(s) as disclosed in any of the above.

In a related aspect, a personalized immunotherapy composition elicits an immune response targeted against one or more neo-epitopes.

In a related aspect, the composition comprises a combination of the Listeria strains, wherein the combination comprises a plurality of the neo-epitopes that are administered on the same day.

In a related aspect the combination comprises a plurality of the Listeria strains that are administered on different days or in alternating sequence wherein the combination of strains administered on different days comprises a plurality of the neo-epitopes.

In a related aspect, the composition comprises a combination of the Listeria strains, wherein the combination comprises a plurality of the neo-epitopes that are administered on the same day.

In a related aspect, the combination comprises all of the neo-epitopes identidied in the patient that can be expressed in this system.

In a related aspect, the combination comprises all or a plurality of of the neo-epitopes described as clonal.

In a related aspect, the combination comprises all or a plurality of the neo-epitopes that are also represented in the transcriptome based on RNA sequencing.

In a related aspect, the composition comprises a combination of a plurality of the Listeria strains, wherein each strain comprises the nucleic acid construct comprising one or more open reading frames encoding one or more peptides comprising at least one unique the neo-epitope.

In a related aspect, the composition comprises a combination of the Listeria strains, wherein the combination comprises a plurality of the neo-epitopes.

In a related aspect, the combination comprises up to about 500 of the neo-epitopes.

In a related aspect, the combination further comprises one or more recombinant attenuated Listeria strain delivery vector comprising a nucleic acid construct comprising one or more open reading frames encoding one or more peptides comprising one or more epitopes, wherein the epitope(s) comprise immunogenic epitope(s) present in a disease-bearing tissue or cell of the subject having the disease or condition, wherein administrating the Listeria strain generates a immunotherapy targeted to the subject's disease or condition.

In a related aspect, the composition, as disclosed in any of the above, further comprising an adjuvant.

In a related aspect, administering the composition to the subject generates a personalized enhanced anti-disease, or anti-condition immune response in the subject.

In a related aspect of the present invention, a DNA immunotherapy comprising the personalized immunotherapy composition as described in any of the above.

In a related aspect of the present invention, a peptide immunotherapy comprising the personalized immunotherapy composition as described in any of the above.

In a related aspect of this invention, a pharmaceutical composition of the present invention comprising the immunotherapy or personalized immunotherapy composition as described in any of the above and a pharmaceutical carrier.

In a related aspect of this invention, a method of inducing an immune response to at least one neo-epitope present in a disease or condition bearing tissue or cell in a subject having the disease or condition, the method comprising the step of administering the personalized immunotherapy composition or immunotherapy as described in any of the above to the subject.

In a related aspect of this invention, a method of inducing a targeted immune response in a subject having a disease or condition, comprising administering to the subject the immunogenic composition or immunotherapy as described in any of the above, wherein administrating the Listeria strain generates a personalized immunotherapy targeted to the subject's disease or condition.

In a related aspect of this invention, a method of treating, suppressing or inhibiting disease or condition in a subject, the method comprising the step of administrating a personalized immunotherapy composition or immunotherapy as described in any of the above, for targeting the disease or condition.

In yet another embodiment, the disease or condition is an infectious disease, autoimmune disease, organ transplantation rejection, a tumor or a cancer.

In a related aspect of the present invention, a method of increasing the ratio of T effector cells to regulatory T cells (Tregs) in the lymphoid tissue or systemic circulation, and tumor, or diseased or dysplastic tissue of a subject, wherein the T effector cells are targeted to a neo-epitope present within a disease or condition bearing tissue of a subject, the method comprising the step of administering to the subject personalized immunotherapy composition or immunotherapy as described in any of the above.

In a related aspect of the present invention, a method for increasing antigen-specific T-cells in a subject, wherein the antigen or a peptide fragment thereof comprises one or more neo-epitopes, the method comprising the step of administering to the subject a personalized immunotherapy composition or immunotherapy as described in any of the above.

In a related aspect of the present invention, a method for increasing survival time of a subject having a tumor or suffering from cancer, or suffering from an infectious disease, comprising the step of administering to the subject a personalized immunotherapy composition or immunotherapy as described in any of the above.

In a related aspect of the present invention, a method of protecting a subject from a cancer, the method comprising the step of administering to the subject a personalized immunotherapy composition or immunotherapy as described in any of the above.

In a related aspect of the present invention, a method of inhibiting or delaying the onset of cancer in a subject, the method comprising the step of administering to the subject a personalized immunotherapy composition or immunotherapy as described in any of the above.

In a related aspect of the present invention, a method of reducing tumor or metastases size in a subject, the method comprising the step of administrating to the subject a personalized immunotherapy composition or immunotherapy as described in any of the above.

In a related aspect of the present invention, a method of protecting a subject from an infectious disease, the method comprising the step of administering to the subject a personalized immunotherapy composition or immunotherapy as described in any of the above.

According to another embodiment of the present invention, a method as described above is disclosed, additionally comprising the steps of creating the personalized immunotherapy composition, wherein the creating comprises the steps of:
(a) comparing one or more open reading frames (ORFs) in nucleic acid sequences extracted from a disease-bearing biological sample with one or more ORFs in nucleic acid sequences extracted from a healthy biological sample, wherein the comparing identifies one or more nucleic acid sequences encoding one or more peptides comprising one or more neo-epitopes encoded within one or more ORFs from the disease-bearing sample;
(b) transforming an attenuated Listeria strain with a vector comprising a nucleic acid sequence encoding one or more peptides comprising one or more neo-epitopes identified in a.; and, alternatively storing the attenuated recombinant Listeria for administering to the subject at a pre-determined period or administering a composition comprising the attenuated recombinant Listeria strain to the subject, and wherein the administering results in the generation of a personalized T-cell immune response against the disease or the condition; optionally,
(c) obtaining a second biological sample from the subject comprising a T-cell clone or T-infiltrating cell from the T-cell immune response and characterizing specific peptides comprising one or more neo-epitopes bound by MHC Class I or MHC Class II molecules on the T cells, wherein one or more neo-epitopes are immunogenic;
(d) screening for and selecting a nucleic acid construct encoding one or more peptides comprising one or more immunogenic neo-epitope identified in (c); and,
(e) transforming a second attenuated recombinant Listeria strain with a vector comprising a nucleic acid sequence encoding one or more peptides comprising one or more immunogenic neo-epitopes; and, alternatively storing the second attenuated recombinant Listeria for administering to the subject at a pre-determined period or administering a second composition comprising the second attenuated recombinant Listeria strain to the subject,
   wherein the process creates a personalized immunotherapy for the subject.

In one embodiment, the invention relates to an immunogenic mixture of compositions comprising one or more recombinant Listeria strains produced by the process disclosed herein. In another embodiment, each of said Listeria in said mixture comprises a nucleic acid molecule encoding a fusion polypeptide or chimeric protein comprising one or more neo-epitopes. In another embodiment, each Listeria in said mixture expresses 1-5, 5-10, 10-15, 15-20, 10-20, 20-30, 30-40, 40-50, 50-60, 60-70, 70-80, 80-90, 90-100, or 100-200 neo-epitopes. In another embodiment, each mixture comprises 1-5, 5-10, 10-15, 15-20, 10-20, 20-30, 30-40, or 40-50 recombinant Listeria strains.

In one embodiment, the invention relates to a method of eliciting a personalized anti-tumor response in a subject, the method comprising the step of concomitantly or sequentially administering to said subject an immunogenic mixture composition disclosed herein. In another embodiment, disclosed herein is a method of preventing or treating a tumor in a subject, the method comprising the step of concomitantly or sequentially administering to said subject the immunogenic mixture of compositions disclosed herein. In one embodiment, the invention relates to a nucleic acid construct encoding a chimeric protein comprising the following elements: a N-terminal truncated LLO (tLLO) fused to a first neo-epitope amino acid sequence, wherein said first neo-epitope AA sequence is operatively linked to a second neo-epitope AA sequence via a linker sequence, wherein said second neo-epitope AA sequence is operatively linked to at least one additional neo-epitope amino acid sequence via a linker sequence, and wherein a last neo-epitope is operatively linked to a histidine tag at the C-terminus via a linker sequence.

In another embodiment, the invention relates to a system for creating personalized immunotherapy for a subject, comprising: at least one processor; and at least one storage medium containing program instructions for execution by said processor, said program instructions causing said processor to execute steps comprising:
a. Receiving output data containing all neo-antigens and the human leukocyte antigen (HLA) type of the subject;
b. Scoring the hydrophobicity of each epitope and removing epitopes that score above a certain threshold;
c. Numerically rate the remaining neo-antigens based its ability to bind to subject HLA and on its predictive MHC binding score;
d. Inserting the amino acid sequence of each epitope into a plasmid;
e. Scoring the hydrophobicity of each construct and removing any constructs that score above a certain threshold;
f. Translating the amino acid sequence of each construct into the corresponding DNA sequence, starting with the highest scored construct;
g. Inserting additional epitopes into the plasmid construct in order of ranking until a predetermined upper limit is reached;
h. Adding a DNA sequence tag to the end of the construct in order to measure the immunotherapeutic response in a subject; and
i. Optimizing the epitope and DNA sequence tag for expression and secretion in Listeria monocytogenes.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A and 1B. Lm-E7 and Lm-LLO-E7 (ADXS11-001) use different expression systems to express and secrete E7. Lm-E7 was generated by introducing a gene cassette into the orfZ domain of the L. monocytogenes genome (FIG. 1A). The hly promoter drives expression of the hly signal sequence and the first five amino acids (AA) of LLO followed by HPV-16 E7. (FIG. 1B), Lm-LLO-E7 was generated by transforming the prfA-strain XFL-7 with the plasmid pGG-55. pGG-55 has the hly promoter driving expression of a non-hemolytic fusion of LLO-E7. pGG-55 also contains the prfA gene to select for retention of the plasmid by XFL-7 in vivo.

FIG. 2. Lm-E7 and Lm-LLO-E7 secrete E7. Lm-Gag (lane 1), Lm-E7 (lane 2), Lm-LLO-NP (lane 3), Lm-LLO-E7 (lane 4), XFL-7 (lane 5), and 10403S (lane 6) were grown overnight at 37° C. in Luria-Bertoni broth. Equivalent numbers of bacteria, as determined by OD at 600 nm absorbance, were pelleted and 18 ml of each supernatant was TCA precipitated. E7 expression was analyzed by Western blot. The blot was probed with an anti-E7 mAb, followed by HRP-conjugated anti-mouse (Amersham), then developed using ECL detection reagents.

FIG. 3. Tumor immunotherapeutic efficacy of LLO-E7 fusions. Tumor size in millimeters in mice is shown at 7, 14, 21, 28 and 56 days post tumor-inoculation. Naive mice: open-circles; Lm-LLO-E7: filled circles; Lm-E7: squares; Lm-Gag: open diamonds; and Lm-LLO-NP: filled triangles.

FIG. 4. Splenocytes from Lm-LLO-E7-immunized mice proliferate when exposed to TC-1 cells. C57BL/6 mice were immunized and boosted with Lm-LLO-E7, Lm-E7, or control rLm strains. Splenocytes were harvested 6 days after the boost and plated with irradiated TC-1 cells at the ratios shown. The cells were pulsed with ³H thymidine and harvested. Cpm is defined as (experimental cpm)—(no-TC-1 control).

FIGS. 5A and 5B. (FIG. 5A) Western blot demonstrating that Lm-ActA-E7 secretes E7. Lane 1: Lm-LLO-E7; lane 2: Lm-ActA-E7.001; lane 3; Lm-ActA-E7-2.5.3; lane 4: Lm-ActA-E7-2.5.4. (FIG. 5B) Tumor size in mice administered Lm-ActA-E7 (rectangles), Lm-E7 (ovals), Lm-LLO-E7 (X), and naive mice (non-vaccinated; solid triangles).

FIGS. 6A-6C. (FIG. 6A) schematic representation of the plasmid inserts used to create 4 LM immunotherapies. Lm-LLO-E7 insert contains all of the Listeria genes used. It contains the hly promoter, the first 1.3 kb of the hly gene (which encodes the protein LLO), and the HPV-16 E7 gene. The first 1.3 kb of hly includes the signal sequence (ss) and the PEST region. Lm-PEST-E7 includes the hly promoter, the signal sequence, and PEST and E7 sequences but excludes the remainder of the truncated LLO gene. Lm-ΔPEST-E7 excludes the PEST region, but contains the hly promoter, the signal sequence, E7, and the remainder of the truncated LLO. Lm-E7epi has only the hly promoter, the signal sequence, and E7. (FIG. 6B) Top panel: Listeria constructs containing PEST regions induce tumor regression. Bottom panel: Average tumor sizes at day 28 post-tumor challenge in 2 separate experiments. (FIG. 6C) Listeria constructs containing PEST regions induce a higher percentage of E7-specific lymphocytes in the spleen. Average and SE of data from 3 experiments are depicted.

FIGS. 7A and 7B. (FIG. 7A) Induction of E7-specific IFN-gamma-secreting CD8⁺ T cells in the spleens and the numbers penetrating the tumors, in mice administered TC-1 tumor cells and subsequently administered Lm-E7, Lm-LLO-E7, Lm-ActA-E7, or no immunotherapy (naive). (FIG. 7B) Induction and penetration of E7 specific CD8⁺ cells in the spleens and tumors of the mice described for (FIG. 7A).

FIGS. 8A and 8B. Listeria constructs containing PEST regions induce a higher percentage of E7-specific lymphocytes within the tumor. (FIG. 8A) representative data from 1 experiment. (FIG. 8B) average and SE of data from all 3 experiments.

FIG. 9. Data from Cohorts 1 and 2 indicting the efficacy observed in the patients in the clinical trial presented in Example 6.

FIGS. 10A and 10B. (FIG. 10A) Schematic representation of the chromosomal region of the Lmdd-143 and LmddA-143 after klk3 integration and actA deletion; (FIG. 10B) The klk3 gene is integrated into the Lmdd and LmddA chromosome. PCR from chromosomal DNA preparation from each construct using klk3 specific primers amplifies a band of 714 bp corresponding to the klk3 gene, lacking the secretion signal sequence of the wild type protein.

FIGS. 11A-11D. (FIG. 11A) Map of the pADV134 plasmid. (FIG. 11B) Proteins from LmddA-134 culture supernatant were precipitated, separated in a SDS-PAGE, and the LLO-E7 protein detected by Western-blot using an anti-E7 monoclonal antibody. The antigen expression cassette consists of hly promoter, ORF for truncated LLO and human PSA gene (klk3). (FIG. 11C) Map of the pADV142 plasmid. (FIG. 11D) Western blot showed the expression of LLO-PSA fusion protein using anti-PSA and anti-LLO antibody.

FIGS. 12A and 12B. (FIG. 12A) Plasmid stability in vitro of LmddA-LLO-PSA if cultured with and without selection pressure (D-alanine). Strain and culture conditions are listed first and plates used for CFU determination are listed after. (FIG. 12B) Clearance of LmddA-LLO-PSA in vivo and assessment of potential plasmid loss during this time. Bacteria were injected i.v. and isolated from spleen at the time point indicated. CFUs were determined on BHI and BHI+D-alanine plates.

FIGS. 13A and 13B. (FIG. 13A) In vivo clearance of the strain LmddA-LLO-PSA after administration of 10⁸ CFU in C57BL/6 mice. The number of CFU were determined by plating on BHI/str plates. The limit of detection of this method was 100 CFU. (FIG. 13B) Cell infection assay of J774 cells with 10403S, LmddA-LLO-PSA and XFL7 strains.

FIGS. 14A-14E. (FIG. 14A) PSA tetramer-specific cells in the splenocytes of naïve and LmddA-LLO-PSA immunized mice on day 6 after the booster dose. (FIG. 14B) Intracellular cytokine staining for IFN-γ in the splenocytes of naïve and LmddA-LLO-PSA immunized mice were stimulated with PSA peptide for 5 h. Specific lysis of EL4 cells pulsed with PSA peptide with in vitro stimulated effector T cells from LmddA-LLO-PSA immunized mice and naïve mice at different effector/target ratio using a caspase based assay (FIG. 14C) and a europium based assay (FIG. 14D). Number of IFNγ spots in naïve and immunized splenocytes obtained after stimulation for 24 h in the presence of PSA peptide or no peptide (FIG. 14E).

FIGS. 15A-15C. Immunization with LmddA-142 induces regression of Tramp-C1-PSA (TPSA) tumors. Mice were left untreated (n=8) (FIG. 15A) or immunized i.p. with LmddA-142 (1×10⁸ CFU/mouse) (n=8) (FIG. 15B) or Lm-LLO-PSA (n=8), (FIG. 15C) on days 7, 14 and 21. Tumor sizes were measured for each individual tumor and the values expressed as the mean diameter in millimeters. Each line represents an individual mouse.

FIGS. 16A and 16B. (FIG. 16A) Analysis of PSA-tetramer⁺CD8⁺ T cells in the spleens and infiltrating T-PSA-23 tumors of untreated mice and mice immunized with either an Lm control strain or LmddA-LLO-PSA (LmddA-142). (FIG. 16B) Analysis of CD4⁺ regulatory T cells, which were defined as CD25⁺FoxP3⁺, in the spleens and infiltrating T-PSA-23 tumors of untreated mice and mice immunized with either an Lm control strain or LmddA-LLO-PSA.

FIGS. 17A and 17B. (FIG. 17A) Schematic representation of the chromosomal region of the Lmdd-143 and LmddA-143 after klk3 integration and actA deletion; (FIG. 17B) The klk3 gene is integrated into the Lmdd and LmddA chromosome. PCR from chromosomal DNA preparation from each construct using klk3 specific primers amplifies a band of 760 bp corresponding to the klk3 gene.

FIGS. 18A-C. (FIG. 18A) Lmdd-143 and LmddA-143 secretes the LLO-PSA protein. Proteins from bacterial culture supernatants were precipitated, separated in a SDS-PAGE and LLO and LLO-PSA proteins detected by Western-blot using an anti-LLO and anti-PSA antibodies; (FIG. 18B) LLO produced by Lmdd-143 and LmddA-143 retains hemolytic activity. Sheep red blood cells were incubated with serial dilutions of bacterial culture supernatants and hemolytic activity measured by absorbance at 590 nm; (FIG. 18C) Lmdd-143 and LmddA-143 grow inside the macrophage-like J774 cells. J774 cells were incubated with bacteria for 1 hour followed by gentamicin treatment to kill extracellular bacteria. Intracellular growth was measured by plating serial dilutions of J774 lysates obtained at the indicated timepoints. Lm 10403S was used as a control in these experiments.

FIG. 19. Immunization of mice with Lmdd-143 and LmddA-143 induces a PSA-specific immune response. C57BL/6 mice were immunized twice at 1-week interval with 1×10⁸ CFU of Lmdd-143, LmddA-143 or LmddA-142 and 7 days later spleens were harvested. Splenocytes were stimulated for 5 hours in the presence of monensin with 1 μM of the PSA₆₅₋₇₄ peptide. Cells were stained for CD8, CD3, CD62L and intracellular IFN-γ and analyzed in a FACS Calibur cytometer.

FIGS. 20A and 20B. Construction of ADXS31-164. (FIG. 20A) Plasmid map of pAdv164, which harbors Bacillus subtilis dal gene under the control of constitutive Listeria p60 promoter for complementation of the chromosomal dal-dat deletion in LmddA strain. It also contains the fusion of truncated LLO₍₁₋₄₄₁₎ to the chimeric human Her2/neu gene, which was constructed by the direct fusion of 3 fragments the Her2/neu: EC1 (aa 40-170), EC2 (aa 359-518) and ICI (aa 679-808). (FIG. 20B) Expression and secretion of tLLO-ChHer2 was detected in Lm-LLO-ChHer2 (Lm-LLO-138) and LmddA-LLO-ChHer2 (ADXS31-164) by western blot analysis of the TCA precipitated cell culture supernatants blotted with anti-LLO antibody. A differential band of ˜104 KD corresponds to tLLO-ChHer2. The endogenous LLO is detected as a 58 KD band. Listeria control lacked ChHer2 expression.

FIGS. 21A-21C. Immunogenic properties of ADXS31-164 (FIG. 21A) Cytotoxic T cell responses elicited by Her2/neu Listeria-based immunotherapies in splenocytes from immunized mice were tested using NT-2 cells as stimulators and 3T3/neu cells as targets. Lm-control was based on the LmddA background that was identical in all ways but expressed an irrelevant antigen (HPV16-E7). (FIG. 21B) IFN-γ secreted by the splenocytes from immunized FVB/N mice into the cell culture medium, measured by ELISA, after 24 hours of in vitro stimulation with mitomycin C treated NT-2 cells. (FIG. 21C) IFN-γ secretion by splenocytes from HLA-A2 transgenic mice immunized with the chimeric immunotherapy, in response to in vitro incubation with peptides from different regions of the protein. A recombinant ChHer2 protein was used as positive control and an irrelevant peptide or no peptide groups constituted the negative controls as listed in the FIG. legend. IFN-γ secretion was detected by an ELISA assay using cell culture supernatants harvested after 72 hours of co-incubation. Each data point was an average of triplicate data+/−standard error. *P value<0.001.

FIG. 22. Tumor Prevention Studies for Listeria-ChHer2/neu Immunotherapies Her2/neu transgenic mice were injected six times with each recombinant Listeria-ChHer2 or a control Listeria immunotherapy Immunizations started at 6 weeks of age and continued every three weeks until week 21. Appearance of tumors was monitored on a weekly basis and expressed as percentage of tumor free mice. *p<0.05, N=9 per group.

FIG. 23. Effect of immunization with ADXS31-164 on the % of Tregs in Spleens. FVB/N mice were inoculated s.c. with 1×10⁶ NT-2 cells and immunized three times with each immunotherapy at one week intervals. Spleens were harvested 7 days after the second immunization. After isolation of the immune cells, they were stained for detection of Tregs by anti CD3, CD4, CD25 and FoxP3 antibodies. Dot-plots of the Tregs from a representative experiment showing the frequency of CD25⁺/FoxP3⁺ T cells, expressed as percentages of the total CD3⁺ or CD3⁺CD4⁺ T cells across the different treatment groups.

FIGS. 24A and 24B. Effect of immunization with ADXS31-164 on the % of tumor infiltrating Tregs in NT-2 tumors. FVB/N mice were inoculated s.c. with 1×10⁶ NT-2 cells and immunized three times with each immunotherapy at one week intervals. Tumors were harvested 7 days after the second immunization. After isolation of the immune cells, they were stained for detection of Tregs by anti CD3, CD4, CD25 and FoxP3 antibodies. (FIG. 24A). Dot-plots of the Tregs from a representative experiment. (FIG. 24B). Frequency of CD25⁺/FoxP3⁺ T cells, expressed as percentages of the total CD3⁺ or CD3⁺CD4⁺ T cells (left panel) and intratumoral CD8/Tregs ratio (right panel) across the different treatment groups. Data is shown as mean±SEM obtained from 2 independent experiments.

FIGS. 25A-25C. Vaccination with ADXS31-164 can delay the growth of a breast cancer cell line in the brain. Balb/c mice were immunized thrice with ADXS31-164 or a control Listeria immunotherapy. EMT6-Luc cells (5,000) were injected intracranially in anesthetized mice. (FIG. 25A) Ex vivo imaging of the mice was performed on the indicated days using a Xenogen X-100 CCD camera. (FIG. 25B) Pixel intensity was graphed as number of photons per second per cm2 of surface area; this is shown as average radiance. (FIG. 25C) Expression of Her2/neu by EMT6-Luc cells, 4T1-Luc and NT-2 cell lines was detected by Western blots, using an anti-Her2/neu antibody. J774.A2 cells, a murine macrophage like cell line was used as a negative control.

FIGS. 26A-C represents a schematic map of a recombinant Listeria protein minigene construct. (FIG. 26A) represents a construct producing the ovalbumin derived SIINFEKL peptide (SEQ ID NO: 75). (FIG. 26B) represents a comparable recombinant protein in which a GBM derived peptide has been introduced in place of SIINFEKL by PCR cloning. (FIG. 26C) represents a construct designed to express 4 separate peptide antigens from a strain of Listeria.

FIG. 27. A schematic representation showing the cloning of the different ActA PEST regions in the plasmid backbone pAdv142 (see FIG. 11C) to create plasmids pAdv211, pAdv223 and pAdv224 is shown in (FIG. 27). This schematic shows different ActA coding regions were cloned in frame with Listeriolysin O signal sequence in the backbone plasmid pAdv142, restricted with XbaI and XhoI.

FIGS. 28A-B. (FIG. 28A) Tumor regression study using TPSA23 as transplantable tumor model. Three groups of eight mice were implanted with 1×10⁶ tumor cells on day 0 and were treated on day 6, 13 and 20 with 10⁸ CFU of different therapies: LmddA142, LmddA211, LmddA223 and LmddA224. Naïve mice did not receive any treatment. Tumors were monitored weekly and mice were sacrificed if the average tumor diameter was 14-18 mm Each symbol in the graph represents the tumors size of an individual mouse. The experiment was repeated twice and similar results were obtained. (FIG. 28B) The percentage survival of the naïve mice and immunized mice at different days of the experiment.

FIGS. 29A-B. PSA specific immune responses were examined by tetramer staining (FIG. 29A) and intracellular cytokine staining for IFN-γ (FIG. 29B). Mice were immunized three times at weekly intervals with 10⁸ CFU of different therapies: LmddA142 (ADXS31-142), LmddA211, LmddA223 and LmddA224. For immune assays, spleens were harvested on day 6 after the second boost. Spleens from 2 mice/group were pooled for this experiment. (A) PSA specific T cells in the spleen of naïve, LmddA142, LmddA211, LmddA223 and LmddA224 immunized mice were detected using PSA-epitope specific tetramer staining. Cells were stained with mouse anti-CD8 (FITC), anti-CD3 (Percp-Cy5.5), anti-CD62L (APC) and PSA tetramer-PE and analyzed by FACS Calibur. (FIG. 29B) Intracellular cytokine staining to detect the percentage of IFN-γ secreting CD8+CD62Llow cells in the naïve and immunized mice after stimulation with 1 μM of PSA specific, H-2Db peptide (HCIRNKSVIL) for 5 h.

FIGS. 30A-C. TPSA23, tumor model was used to study immune response generation in C57BL6 mice by using ActA/PEST2 (LA229) fused PSA and tLLO fused PSA. Four groups of five mice were implanted with 1×10⁶ tumor cells on day 0 and were treated on day 6 and 14 with 10⁸ CFU of different therapies: LmddA274, LmddA142 (ADXS31-142) and LmddA211. Naïve mice did not receive any treatment. On Day 6 post last immunization, spleen and tumor was collected from each mouse. (FIG. 30A) Table shows the tumor volume on day 13 post immunization. PSA specific immune responses were examined by pentamer staining in spleen (FIG. 30B) and in tumor (FIG. 30C). For immune assays, spleens from 2 mice/group or 3 mice/group were pooled and tumors from 5 mice/group was pooled. Cells were stained with mouse anti-CD8 (FITC), anti-CD3 (Percp-Cy5.5), anti-CD62L (APC) and PSA Pentamer-PE and analyzed by FACS Calibur.

FIGS. 31A-31C. SOE mutagenesis strategy. Decreasing/lowering the virulence of LLO was achieved by mutating the 4th domain of LLO. (FIGS. 31A-31B). This domain contains a cholesterol binding site allowing it to bind to membranes where it oligomerizes to form pores. FIG. 31C Shows fragments of full length LLO (rLLO529). Recombinant LLO, rLLO493, represents a LLO N-terminal fragment spanning from amino acids 1-493 (including the signal sequence). Recombinant LLO, rLLO482, represents an N-terminal LLO fragment (including a deletion of the cholesterol binding domain, amino acids 483-493) spanning from amino acids 1-482 (including the signal sequence). Recombinant LLO, rLLO415, represents an N-terminal LLO fragment (including a deletion of the cholesterol binding domain, amino acids 483-493) spanning from amino acids 1-415 (including the signal sequence). Recombinant LLO, rLLO59-415, represents an N-terminal LLO fragment that spans from amino acids 59-415 (excluding the cholesterol binding domain). Recombinant LLO, rLLO416-529, represents a N-terminal LLO fragment that spans from amino acids 416-529 and includes the cholesterol binding domain.

FIGS. 32A and 32B. Expression of mutant LLO proteins by Coomassie staining is shown in FIG. 32A and by Western blot in FIG. 32B.

FIGS. 33A and 33B. Histograms present data showing hemolytic activity of mutant LLO (mutLLO and ctLLO) proteins at pH 5.5 (FIG. 33A) and 7.4 (FIG. 33B).

FIG. 34. A plasmid map of a PAK6 construct (7605 bp), wherein PAK6 is expressed as a fusion protein with tLLO. Schematic map of the plasmid for PAK6. The plasmid contains both Listeria (Rep R) and Escherichia coli (p15) origin of replication. The black arrow represents the direction of transcription. Bacillus subtilis dal gene complements the synthesis of D-alanine. The antigen expression cassette consists of hly promoter, ORF for truncated LLO and human PAK6 gene.

FIG. 35. A nucleic acid sequences of PAK6 as set forth in SEQ ID NO: 102.

FIG. 36. An amino acid sequence of PAK6 as set forth in SEQ ID NO: 103.

FIG. 37A. General overview of the tumor sequencing and DNA generation work stream.

FIG. 37B. General overview of DNA cloning and immunotherapy manufacturing work stream.

FIG. 38. Diagram of a cluster of fully enclosed single use cell growth systems arranged for parallel manufacturing of personalized immunotherapy compositions.

FIG. 39. Detailed diagram of the inoculation and fermentation segments of fully enclosed single use cell growth system.

FIG. 40. Detailed diagram of the concentration segment of fully enclosed single use cell growth system.

FIG. 41. Detailed diagram of the diafiltration segment of fully enclosed single use cell growth system.

FIG. 42. Detailed diagram of the product dispensation segment of fully enclosed single use cell growth system.

FIG. 43A. Diagram of the process of using a serial selection of neo-epitopes in order to improve efficiency of immunotherapy.

FIG. 43B. Diagram of the process of using a parallel selection multiple neo-epitopes.

FIG. 44. Flow chart of a process (manual or automated) that generates the DNA sequence of a personalized plasmid vector comprising one or more neo-epitopes for use in a delivery vector, e.g., Listeria monocytogenes using output data containing all neo-antigens and patient HLA types.

FIG. 45 shows the effects of moving the SIINFEKL tag on 25D detection. The SIINFEKL tag identifies a secreted neo-epitope whether the tag is located at the C-terminus, the N-terminus, or in between.

FIG. 46A shows the timeline for B16F10 tumor experiments, including treatments with Lm Neo constructs.

FIG. 46B shows tumor regression with LmddA274, Lm-Neo-12, and Lm-Neo-20, with PBS used as a negative control.

FIG. 46C compares survival of mice with B16F10 tumors following treatment with LmddA274, Lm-Neo-12, or Lm-Neo-20, with PBS used as a negative control.

FIG. 47A-C show expression and secretion levels for PSA-Survivin-SIINFEKL (FIG. 47A), PSA-Survivin without SIINFEKL (FIG. 47B), and Neo 20-SIINFEKL (FIG. 47C).

FIG. 48 shows CD8 T-cell response to the Neo 20 antigen (with C-terminal SIINFEKL tag) or a negative control. The graph indicates the percent SIINFEKL-specific CD8 T-cell response for each condition.

FIG. 49A shows tumor regression with LmddA274, Lm-Neo-12, Lm-Neo-20, and Lm-Neo 30, with PBS used as a negative control.

FIG. 49B compares survival of mice with B16F10 tumors following treatment with LmddA274, Lm-Neo-12, Lm-Neo-20, and Lm-Neo 30, with PBS used as a negative control.

FIG. 50 shows the effects of randomizing the order of neo-epitopes within a construct or breaking down the combination of neo-epitopes into subcombinations of neo-epitopes and randomizing those subcombinations to modify secretion.

FIG. 51 shows the relative CD8 cell response in mice immunized with lung neo-epitope constructs.

It will be appreciated that for simplicity and clarity of illustration, elements shown in the Figs. have not necessarily been drawn to scale. For example, the dimensions of some of the elements may be exaggerated relative to other elements for clarity. Further, where considered appropriate, reference numerals may be repeated among the Figs. to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details, as embodied herein. In other instances, well-known methods, procedures, and components have not been described in detail so as not to obscure the present invention.

In one embodiment, provided herein is a system for providing a personalized immunotherapy system created for a subject having a disease or condition, said system comprising:
a. an attenuated Listeria strain delivery vector; and
b. a plasmid vector for transforming said Listeria strain, said plasmid vector comprising a nucleic acid construct comprising one or more open reading frames encoding one or more peptides comprising one or more neo-epitopes, wherein said neo-epitope(s) comprise immunogenic epitopes present in a disease-bearing tissue or cell of said subject having said disease or condition;
   wherein transforming said Listeria strain with said plasmid vector creates a personalized immunotherapy system targeted to said subject's disease or condition.

In one embodiment, the present invention provides a process for creating a personalized immunotherapy for a subject having a disease or condition, the process comprising the steps of:
a. comparing one or more open reading frames (ORFs) in nucleic acid sequences extracted from a disease-bearing biological sample with one or more ORFs in nucleic acid sequences extracted from a healthy biological sample, wherein said comparing identifies one or more neo-epitopes encoded within said one or more ORFs from the disease-bearing sample;
b. screening peptides comprising said one or more neo-epitopes for an immunogenic response;
c. transforming an attenuated Listeria strain with a plasmid vector comprising a nucleic acid sequence that encodes a one or more peptides comprising said one or more immunogenic neo-epitopes; and
d. alternatively storing said attenuated recombinant Listeria for administering to said subject at a pre-determined period or administering said attenuated recombinant Listeria strain to said subject, wherein said attenuated recombinant Listeria strain is administered as part of an immunogenic composition.

In another embodiment, provided herein is a system for providing a personalized immunotherapy for a subject having a disease or condition, comprising the following components:
a. a disease-bearing biological sample obtained from said subject having said disease or condition;
b. a healthy biological sample, wherein said healthy biological sample is obtained from said human subject having said disease or condition or another healthy human subject;
c. a screening assay or screening tool and associated digital software for comparing one or more open reading frames (ORFs) in nucleic acid sequences extracted from said disease-bearing biological sample with open reading frames in nucleic acid sequences extracted from said healthy biological sample, and for identifying mutations in said ORFs encoded by said nucleic acid sequences of said disease-bearing sample, wherein said mutations comprise one or more neo-epitopes;
   i. wherein said associated digital software comprises access to a sequence database that allows screening of said mutations within said ORFs for identification of T-cell epitope(s) or immunogenic potential, or any combination thereof;
d. a nucleic acid cloning and expression kit for cloning and expressing a nucleic acid encoding one or more peptides comprising said one or more neo-epitopes from said disease-bearing sample;
e. an immunogenic assay for testing the T-cell immunogenecity of candidate peptides comprising one or more neo-epitopes;
f. an attenuated Listeria delivery vector for transforming with a plasmid vector comprising a nucleic acid construct comprising one or more open reading frames encoding said identified immunogenic peptides comprising one or more immunogenic neo-epitopes of step (e),
   wherein once transformed, said Listeria is stored or is administered to said human subject in (a) as part of an immunogenic composition.

In another embodiment, an infectious disease, an organ transplant rejection, or a tumor or cancer.

In one embodiment, the present invention relates to a system for providing a personalized immunotherapy system created for a subject having a disease or condition, said system comprising:
c. a delivery vector; and optionally
d. a plasmid vector for transforming said delivery vector, said plasmid vector comprising a nucleic acid construct comprising one or more open reading frames encoding one or more peptides comprising one or more neo-epitopes, wherein said neo-epitope(s) comprise immunogenic epitopes present in a disease-bearing tissue or cell of said subject having said disease or condition.

In one embodiment, provided herein is a recombinant attenuated Listeria strain, wherein the Listeria strain comprises a nucleic acid sequence comprising one or more open reading frames encoding one or more peptides comprising one or more personalized neo-epitopes, wherein the neo-epitopes comprise immunogenic epitopes present in a disease- or condition-bearing tissue or cell of a subject having the disease or condition.

In one embodiment, provided herein is a recombinant attenuated Listeria strain comprising: (a) a nucleic acid molecule, the nucleic acid molecule comprising a first open reading frame encoding a fusion polypeptide, wherein the fusion polypeptide comprises an immunogenic polypeptide or fragment thereof fused to one or more peptides comprising one or more neo-epitopes provided herein; or (b) a minigene nucleic acid construct comprising one or more open reading frames encoding a chimeric protein, wherein the chimeric protein comprises: (i) a bacterial secretion signal sequence; (ii) a ubiquitin (Ub) protein; and (iii) one or more peptides comprising one or more neo-epitopes provided herein; wherein the signal sequence, the ubiquitin, and the one or more peptides in (i)-(iii) are operatively linked or arranged in tandem from the amino-terminus to the carboxy-terminus, wherein the neo-epitopes comprise immunogenic epitopes present in a disease- or condition-bearing tissue or cell of a subject having the disease or condition.

In another embodiment, administrating the Listeria strain to a subject having said disease or condition generates an immune response targeted to the subject's disease or condition.

In another embodiment, the strain is a personalized immunotherapy vector for said subject targeted to said subject's disease or condition.

In another embodiment, the peptides comprise at least two different neo-epitope amino acid sequences.

In another embodiment, the peptides comprise one or more neo-epitope repeats of the same amino acid sequence.

In another embodiment, the Listeria strain comprises one neo-epitope.

In another embodiment, the Listeria strain comprises the neo-epitopes in the range of about 1-100. Alternatively, the Listeria strain comprises the neo-epitopes in the range of about 1-5, 5-10, 10-15, 15-20, 10-20, 20-30, 30-40, 40-50, 50-60, 60-70, 70-80, 80-90, 90-100, 5-15, 5-20, 5-25, 15-20, 15-25, 15-30, 15-35, 20-25, 20-35, 20-45, 30-45, 30-55, 40-55, 40-65, 50-65, 50-75, 60-75, 60-85, 70-85, 70-95, 80-95, 80-105 or 95-105. Alternatively, the Listeria strain comprises the neo-epitopes in the range of about 50-100. Alternatively, the Listeria strain comprises up to about 100 neo-epitopes. Alternatively, the Listeria strain comprises the neo-epitopes in the range of about 1-100, 5-100, 5-75, 5-50, 5-40, 5-30, 5-20, 5-15 or 5-10. Alternatively, the Listeria strain comprises the neo-epitopes in the range of about 1-100, 1-75, 1-50, 1-40, 1-30, 1-20, 1-15 or 1-10.

In another embodiment, the Listeria strain comprises above about 100 neo-epitopes. In another embodiment, the Listeria strain comprises up to about 10 neo-epitopes. In another embodiment, the Listeria strain comprises up to about 20 neo-epitopes. In another embodiment, the Listeria strain comprises up to about 30 neo-epitopes. In another embodiment, the Listeria strain comprises up to about 40 neo-epitopes. In another embodiment, the Listeria strain comprises up to about 50 neo-epitopes. Alternatively, the Listeria strain comprises about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 neo-epitopes.

In one embodiment described herein, incorporation of amino acids in the range of about 5-30 amino acids flanking each side of a detected mutation in a neo-epitope are generated. Additionally or alternatively, varying sizes of neo-epitope inserts are inserted in the range of about 8-27 amino acids in length. Additionally or alternatively, varying sizes of neo-epitope inserts are inserted in the range of about 5-50 amino acids in length. Additionally or alternatively, varying sizes of neo-epitope inserts (i.e., a peptide encoding a neo-epitope) are inserted in the range of 10-30, 10-40, 15-30, 15-40, or 15-25 amino acids in length. In another embodiment each neo-epitope insert is 1-10, 10-20, 20-30, or 30-40 amino acids long. In another embodiment, the neo-epitope insert is 1-100, 5-100, 5-75, 5-50, 5-40, 5-30, 5-20, 5-15 or 5-10 amino acids long. In yet another embodiment, the neo-epitope amino acid sequence is 1-100, 1-75, 1-50, 1-40, 1-30, 1-20, 1-15 or 1-10. In another embodiment, each neo-epitope insert is 21 amino acids in length or is a “21-mer” neo-epitope sequence. In yet another embodiment, the neo-epitope amino acid insert is about 8-11 or 11-16 amino acids long.

In another embodiment, the neo-epitope sequences are tumor-specific, metastasis-specific, bacterial-infection-specific, viral-infection-specific, and any combination thereof. Additionally or alternatively, the neo-epitope sequences are inflammation-specific, immune-regulation-molecule-epitope-specific, T-cell-specific, an autoimmune-disease-specific, Graft-versus-host disease-(GvHD)-specific, and any combination thereof.

In another embodiment, one or more neo-epitopes comprise linear neo-epitopes. Additionally or alternatively, one or more neo-epitopes comprise a solvent-exposed epitope. In another embodiment, one or more neo-epitopes comprise conformational neo-epitopes.

In another embodiment, one or more neo-epitopes comprise a T-cell epitope.

In one embodiment, disclosed herein is a nucleic acid construct encoding a chimeric protein comprising the following elements: an immunogenic polypeptide fused to a first neo-epitope amino acid (AA) sequence, wherein said first neo-epitope AA sequence is operatively linked to a second neo-epitope AA sequence via a linker sequence, wherein said second neo-epitope AA sequence is operatively linked to at least one additional neo-epitope amino acid sequence via a linker sequence. Optionally, the immunogenic polypeptide is an N-terminal truncated LLO (tLLO). Optionally, the last neo-epitope is operatively linked to a tag, such as a histidine tag at the C-terminus, via a linker sequence. Optionally, the nucleic acid construct comprises at least 1 stop codon (e.g., 2 stop codons) following the sequence encoding the tag. In one embodiment, disclosed herein is a nucleic acid construct encoding a chimeric protein comprising the following elements: a N-terminal truncated LLO (tLLO) fused to a first neo-epitope amino acid (AA) sequence, wherein said first neo-epitope AA sequence is operatively linked to a second neo-epitope AA sequence via a linker sequence, wherein said second neo-epitope AA sequence is operatively linked to at least one additional neo-epitope amino acid sequence via a linker sequence, and wherein a last neo-epitope is operatively linked to a histidine tag at the C-terminus via a linker sequence. Optionally, the histidine tag is a 6× histidine tag. In another embodiment, said elements are arranged or are operatively linked from N-terminus to C-terminus. In another embodiment, each nucleic acid construct comprises at least 1 stop codon following the sequence encoding said 6× histidine (HIS) tag.

In another embodiment, each nucleic acid construct comprises 2 stop codons following the sequence encoding said 6× histidine (HIS) tag. In another embodiment, said 6× histidine tag is operatively linked at the N-terminus to a SIINFEKL peptide. In another embodiment, said linker is a 4× glycine linker.

In another embodiment, the nucleic acid construct comprises at least one additional neo-epitope amino acid sequence. In another embodiment, the nucleic acid construct comprises 2-10 additional neo-epitopes, 10-15 additional neo-epitopes, 10-25 additional neo-epitopes, 25-40 additional neo-epitopes, or 40-60 additional neo-epitopes. In another embodiment, the nucleic acid construct comprises about 1-10, about 10-30, about 30-50, about 50-70, about 70-90, or up to about 100 neo-epitopes. For example, the nucleic acid construct can comprise about 5-100 neo-epitopes, or about 15-35 neo-epitopes.

In another embodiment each neo-epitope amino acid sequence is 1-10, 10-20, 20-30, or 30-40 amino acids long. In another embodiment, the neo-epitope amino acid sequence is 1-100, 5-100, 5-75, 5-50, 5-40, 5-30, 5-20, 5-15 or 5-10 amino acids long. In yet another embodiment, the neo-epitope amino acid sequence is 1-100, 1-75, 1-50, 1-40, 1-30, 1-20, 1-15 or 1-10. In another embodiment, each neo-epitope amino acid sequence is 21 amino acids in length or is a “21-mer” neo-epitope sequence. In yet another embodiment, the neo-epitope amino acid sequence is about 8-11 or 11-16 amino acids long.

In another embodiment, the nucleic acid construct encodes a recombinant polypeptide, chimeric protein, or fusion polypeptide comprising an N-terminal truncated LLO fused to a 21 amino acid sequence of a neo-epitope flanked by a linker sequence and followed by at least one second neo epitope flanked by another linker and terminated by a SIINFEKL-6×His tag- and 2 stop codons closing the open reading frame: pHly-tLLO-21mer #1-4× glycine linker G1-21mer #2-4× glycine linker G2- . . . -SIINFEKL-6×His tag-2× stop codon. In another embodiment, expression of the above construct is driven by an hly promoter.

In another embodiment, the nucleic acid sequence comprises one or more linker sequences incorporated between at least one first neo-epitope and at least one second neo-epitope. In another embodiment, the nucleic acid sequence comprises at least two different linker sequences incorporated between at least one first neo-epitope and at least one second neo-epitope to at least one third epitope. In another embodiment, one or more linker(s) is a 4× glycine linker selected from a group comprising nucleotide sequences as set forth in SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, and SEQ ID NO: 86.

In another embodiment, the nucleic acid sequence comprises at least one sequence encoding a TAG fused to the encoded peptide. In another embodiment, the TAG comprises the amino acid sequence as set forth in SEQ ID NO: 87.

In another embodiment, the one or more neo-epitopes each comprises between about 8 to 27 amino acids. Alternatively, the one or more neo-epitopes each comprises between about 5 to 50 amino acids. In another embodiment, the one or more neo-epitopes each comprises about 21 amino acids

In another embodiment, the neo-epitopes are determined using exome sequencing or transcriptome sequencing of the disease-bearing tissue or cell.

In another embodiment, the neo-epitopes comprise a nucleic acid sequence encoding a selected amino acid mutation in comparison to a matching biological sample amino acid sequences, flanked by about 10 amino acids on its N-terminus and about 10 amino acids on its C-terminus.

In another embodiment, one or more neo-epitopes, peptides comprising the immunogenic epitopes, or both, are hydrophilic.

In another embodiment, one or more neo-epitopes, peptides comprising the immunogenic epitopes, or both are up to 1.6 on the Kyte Doolittle hydropathy plot.

In another embodiment, one or more neo-epitope(s) are screened for immunosuppressive epitopes, wherein immunosuppressive epitopes are excluded from the nucleic acid molecule.

In another embodiment, one or more neo-epitope(s) are codon optimized for expression and secretion according to the Listeria strain.

In one embodiment, the nucleic acid sequence encoding a neo-epitope, therapeutic polypeptide or nucleic acid is optimized for increased levels of one or more neo-epitope or nucleic acid expression, or, in another embodiment, for increased duration of therapeutic polypeptide comprising one or more neo-epitopes or nucleic acid expression, or, in another embodiment, a combination thereof. Additionally or alternatively, the nucleic acid sequence encoding a neo-epitope, therapeutic polypeptide or nucleic acid is optimized for increased levels of translation, secretion, transcription, and any combination thereof.

Additionally or alternatively, the nucleic acid sequence encoding a neo-epitope, therapeutic polypeptide or nucleic acid is optimized for nucleic acid sequence encoding a neo-epitope, therapeutic polypeptide or nucleic acid is optimized for decreased levels of secondary structures possibilities possibly formed in the oligonucleotide sequence, or alternatively optimized to prevent attachment of any enzyme that may modify the sequence.

In one embodiment, the term “optimized” refers to a desired change, which, in one embodiment, is a change in synthetic gene expression comprising one or more neo-epitopes as described in the present invention, and, in another embodiment, in protein expression. In one embodiment, optimized gene expression is optimized regulation of gene expression. In another embodiment, optimized gene expression is an increase in gene expression. According to this aspect and in one embodiment, a 2-fold through 1000-fold increase in gene expression compared to wild-type is contemplated. In another embodiment, a 2-fold to 500-fold increase in gene expression, in another embodiment, a 2-fold to 100-fold increase in gene expression, in another embodiment, a 2-fold to 50-fold increase in gene expression, in another embodiment, a 2-fold to 20-fold increase in gene expression, in another embodiment, a 2-fold to 10-fold increase in gene expression, in another embodiment, a 3-fold to 5-fold increase in gene expression is contemplated.

In another embodiment, optimized gene expression may be an increase in gene expression under particular environmental conditions. In another embodiment, optimized gene expression may comprise a decrease in gene expression, which, in one embodiment, may be only under particular environmental conditions.

In another embodiment, optimized synthetic gene expression is an increased duration of gene expression. According to this aspect and in one embodiment, a 2-fold through 1000-fold increase in the duration of gene expression compared to wild-type is contemplated. In another embodiment, a 2-fold to 500-fold increase in the duration of gene expression, in another embodiment, a 2-fold to 100-fold increase in the duration of gene expression, in another embodiment, a 2-fold to 50-fold increase in the duration of gene expression, in another embodiment, a 2-fold to 20-fold increase in the duration of gene expression, in another embodiment, a 2-fold to 10-fold increase in the duration of gene expression, in another embodiment, a 3-fold to 5-fold increase in the duration of gene expression is contemplated. In another embodiment, the increased duration of gene expression is compared to gene expression in non-vector-expressing controls, or alternatively, compared to gene expression in wild-type-vector-expressing controls.

Expression in bacterial cells is hampered, in one embodiment, by transcriptional silencing, low mRNA half-life, secondary structure formation, attachment sites of oligonucleotide binding molecules such as repressors and inhibitors, and availability of rare tRNAs pools. The source of many problems in bacterial expressions is found within the original sequence. The optimization of RNAs may include modification of cis acting elements, adaptation of its GC-content, modifying codon bias with respect to non-limiting tRNAs pools of the bacterial cell, and voiding internal homologous regions.

Therefore, in one embodiment, when relying on carefully designed synthetic sequences, stable messages with prolonged half-lives, high level protein production within the host can be expected.

Thus, in one embodiment, optimizing a sequence entails adapting the codon usage to the codon bias of host genes, which in one embodiment, are Listeria monocytogenes genes; adjusting regions of very high (>80%) or very low (<30%) GC content; avoiding one or more of the following cis-acting sequence motifs: internal TATA-boxes, chi-sites and ribosomal entry sites; AT-rich or GC-rich sequence stretches; repeat sequences and RNA secondary structures; (cryptic) splice donor and acceptor sites, branch points; or a combination thereof. In one embodiment, a gene is optimized for expression in Homo sapiens cells. In still another embodiment, optimizing expression entails adding sequence elements to flanking regions of a gene and/or elsewhere in the expression vector.

In one embodiment, the formulations and methods of the present invention provide a nucleic acid optimized for increased expression levels, duration, or a combination thereof of a therapeutic polypeptide comprising one or more neo-epitope encoded by said nucleic acid.

In another embodiment, one or more neo-epitope(s) allow for MHC class II epitope presentation.

In another embodiment, the Listeria strain expresses and secretes one or more peptides comprising one or more neo-epitopes.

In another embodiment, the Listeria strain expresses and secretes one or more peptides comprising one or more neo-epitopes during infection of the subject.

In another embodiment, the Listeria strain comprises a plurality of the nucleic acid sequence molecules.

In one embodiment, the nucleic acid construct encoding the fusion polypeptides disclosed herein is a plasmid insert. In another embodiment, the insert comprises a first open reading frame encoding said fusion polypeptide. In another embodiment, the fusion polypeptide comprises an immunogenic polypeptide or fragment thereof fused to one or more peptides comprising one or more neo-epitopes disclosed herein. In an embodiment, this insert may be on a plasmid, or at least partially integrated into the genome. In another embodiment the insert can be designed as a minigene nucleic acid construct comprising one or more open reading frames encoding a chimeric protein, the chimeric protein includes: a bacterial secretion signal sequence, an ubiquitin (Ub) protein, and one or more peptides comprising one or more neo-epitopes provided herein. In another embodiment, the signal sequence, said ubiquitin and one or more peptides are operatively linked or arranged in tandem from the amino-terminus to the carboxy-terminus.

In another embodiment, the Listeria strain comprises the nucleic acid sequence in a minigene nucleic acid construct comprising one or more open reading frames encoding a chimeric protein, wherein the chimeric protein comprises: (a) a bacterial secretion signal sequence, (b) a ubiquitin (Ub) protein, (c) one or more peptides comprising one or more neo-epitopes provided herein; and, wherein the signal sequence, the ubiquitin, and the one or more peptides in (a)-(c) are operatively linked or arranged in tandem from the amino-terminus to the carboxy-terminus.

In another embodiment, the nucleic acid molecule is in a bacterial artificial chromosome in the recombinant Listeria strain.

In another embodiment, the nucleic acid molecule is in a plasmid in the recombinant Listeria strain.

In another embodiment, the plasmid is an integrative plasmid.

In another embodiment, the plasmid is an extrachromosomal multicopy plasmid.

In another embodiment, the plasmid is stably maintained in the Listeria strain in the absence of antibiotic selection.

In another embodiment, the plasmid does not confer antibiotic resistance upon the recombinant Listeria.

In another embodiment, the one or more peptides are each fused to an immunogenic polypeptide or fragment thereof. For example, each of the one or more peptides can be fused to different immunogenic polypeptides or fragments thereof, or the combination of the one or more peptides can be fused to an immunogenic polypeptide or fragment thereof (e.g., an immunogenic polypeptide linked to a first neo-epitope, which is linked to a second neo-epitope, which is linked to a third neo-epitope, and so forth).

In another embodiment, the one or more peptides comprising one or more immunogenic neo-epitopes are fused concomitantly to an immunogenic polypeptide or fragment thereof.

In another embodiment, the immunogenic polypeptide is a mutated Listeriolysin O (LLO) protein, a truncated LLO (tLLO) protein, a truncated ActA protein, an ActA-PEST2 fusion, or a PEST amino acid sequence.

In another embodiment, the ActA-PEST2 fusion protein is set forth in SEQ ID NO: 16.

In another embodiment, the tLLO protein is set forth in SEQ ID NO: 3.

In another embodiment, the actA is set forth in SEQ ID NO: 12-13 and 15-18.

In another embodiment, the PEST amino acid sequence is selected from the sequences set forth in SEQ ID NOs: 5-10.

In another embodiment, the mutated LLO comprises a mutation in a cholesterol-binding domain (CBD).

In another embodiment, the mutation comprises a substitution of residue C484, W491, or W492 of SEQ ID NO: 2, or any combination thereof.

In another embodiment, the mutation comprises a substitution of 1-11 amino acid(s) within the CBD set forth in SEQ ID NO: 68 with a 1-50 amino acid non-LLO peptide, wherein the non-LLO peptide comprises a peptide comprising a neo-epitope.

In another embodiment, the mutation comprises a deletion of 1-11 amino acid(s) within the CBD as set forth in SEQ ID NO: 68.

In another embodiment, the one or more peptides comprise a heterologous antigen or a self-antigen associated with said disease. In another embodiment, the heterologous antigen or the self-antigen is a tumor-associated antigen or a fragment thereof.

In another embodiment, the neo-epitope or fragment thereof comprises a Human Papilloma Virus (HPV)-16-E6, HPV-16-E7, HPV-18-E6, HPV-18-E7, a Her/2-neu antigen, a chimeric Her2 antigen, a Prostate Specific Antigen (PSA), bivalent PSA, ERG, Androgen receptor (AR), PAK6, Prostate Stem Cell Antigen (PSCA), NY-ESO-1, a Stratum Corneum Chymotryptic Enzyme (SCCE) antigen, Wilms tumor antigen 1 (WT-1), HIV-1 Gag, human telomerase reverse transcriptase (hTERT), Proteinase 3, Tyrosinase Related Protein 2 (TRP2), High Molecular Weight Melanoma Associated Antigen (HMW-MAA), synovial sarcoma, X (SSX)-2, carcinoembryonic antigen (CEA), Melanoma-Associated Antigen E (MAGE-A, MAGE 1, MAGE2, MAGE3, MAGE4), interleukin-13 Receptor alpha (IL13-R alpha), Carbonic anhydrase IX (CALX), survivin, GP100, an angiogenic antigen, a ras protein, a p53 protein, a p97 melanoma antigen, KLH antigen, carcinoembryonic antigen (CEA), gp100, MART1 antigen, TRP-2, HSP-70, beta-HCG, or Testisin.

In another embodiment, the tumor or cancer comprises a breast cancer or tumor, a cervical cancer or tumor, an Her2 expressing cancer or tumor, a melanoma, a pancreatic cancer or tumor, an ovarian cancer or tumor, a gastric cancer or tumor, a carcinomatous lesion of the pancreas, a pulmonary adenocarcinoma, a glioblastoma multiforme, a colorectal adenocarcinoma, a pulmonary squamous adenocarcinoma, a gastric adenocarcinoma, an ovarian surface epithelial neoplasm, an oral squamous cell carcinoma, non-small-cell lung carcinoma, an endometrial carcinoma, a bladder cancer or tumor, a head and neck cancer or tumor, a prostate carcinoma, a renal cancer or tumor, a bone cancer or tumor, a blood cancer, or a brain cancer or tumor.

In another embodiment, the tumor or cancer comprises a metastasis of the tumor or cancer.

In another embodiment, the disease or condition is an infectious disease, an autoimmune disease, or a tumor or a cancer.

In another embodiment, the infectious disease comprises a viral or bacterial infection.

In another embodiment, one or more neo-epitopes comprise an infectious disease-associated-specific epitope.

In another embodiment, the infections disease is an infectious viral disease.

In another embodiment, the infections disease is an infectious bacterial disease.

In another embodiment, the infectious disease is caused by one of the following pathogens: Leishmania, Entamoeba histolytica (which causes amebiasis), Trichuris, BCG/Tuberculosis, Malaria, Plasmodium falciparum, Plasmodium malariae, Plasmodium vivax, Rotavirus, Cholera, Diptheria-Tetanus, Pertussis, Haemophilus influenzae, Hepatitis B, Human papilloma virus, Influenza seasonal), Influenza A (H1N1) Pandemic, Measles and Rubella, Mumps, Meningococcus A+C, Oral Polio Immunotherapies, mono, bi and trivalent, Pneumococcal, Rabies, Tetanus Toxoid, Yellow Fever, Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers, Arenaviruses (LCM, Junin virus, Machupo virus, Guanarito virus, Lassa Fever), Bunyaviruses (Hantaviruses, Rift Valley Fever), Flaviruses (Dengue), Filoviruses (Ebola, Marburg), Burkholderia pseudomallei, Coxiella burnetii (Q fever), Brucella species (brucellosis), Burkholderia mallei (glanders), Chlamydia psittaci (Psittacosis), Ricin toxin (from Ricinus communis), Epsilon toxin of Clostridium perfringens, Staphylococcus enterotoxin B, Typhus fever (Rickettsia prowazekii), other Rickettsias, Food- and Waterborne Pathogens, Bacteria (Diarrheagenic E. coli, Pathogenic Vibrios, Shigella species, Salmonella BCG/, Campylobacter jejuni, Yersinia enterocolitica), Viruses (Caliciviruses, Hepatitis A, West Nile Virus, LaCrosse, Calif. encephalitis, VEE, EEE, WEE, Japanese Encephalitis Virus, Kyasanur Forest Virus, Nipah virus, hantaviruses, Tickborne hemorrhagic fever viruses, Chikungunya virus, Crimean-Congo Hemorrhagic fever virus, Tickborne encephalitis viruses, Hepatitis B virus, Hepatitis C virus, Herpes Simplex virus (HSV), Human immunodeficiency virus (HIV), Human papillomavirus (HPV)), Protozoa (Cryptosporidium parvum, Cyclospora cayatanensis, Giardia lamblia, Entamoeba histolytica, Toxoplasma), Fungi (Microsporidia), Yellow fever, Tuberculosis, including drug-resistant TB, Rabies, Prions, Severe acute respiratory syndrome associated coronavirus (SARS-CoV), Coccidioides posadasii, Coccidioides immitis, Bacterial vaginosis, Chlamydia trachomatis, Cytomegalovirus, Granuloma inguinale, Hemophilus ducreyi, Neisseria gonorrhea, Treponema pallidum, Streptococcus mutans, or Trichomonas vaginalis.

In another embodiment, the attenuated Listeria comprises a mutation in one or more endogenous genes.

In another embodiment, the endogenous gene mutation is selected from an actA gene mutation, a prfA mutation, an actA and inlB double mutation, a dal/dal gene double mutation, or a dal/dat/actA gene triple mutation, or a combination thereof.

In another embodiment, the mutation comprises an inactivation, truncation, deletion, replacement or disruption of the gene or genes.

In another embodiment, the vector further comprises an open reading frame or a second nucleic acid sequence comprising an open reading frame encoding a metabolic enzyme.

In another embodiment, the metabolic enzyme encoded by the open reading frame is an alanine racemase enzyme or a D-amino acid transferase enzyme.

In another embodiment, the Listeria is Listeria monocytogenes.

In another embodiment, the Listeria strain further comprises a nucleic acid construct comprising one or more open reading frames encoding one or more one or more immunomodulatory molecule(s).

In another embodiment, the immunomodulatory molecule is expressed and secreted from said Listeria strain, wherein said molecule is selected from a group comprising Interferon gamma, a cytokine, a chemokine, a T-cell stimulant, and any combination thereof.

In one embodiment, a personalized immunotherapy composition disclosed herein comprises one or more delivery vectors as disclosed herein. In one embodiment, a personalized immunotherapy composition disclosed herein comprises one or more Listeria strain(s) as disclosed in any of the above. In another embodiment, a personalized immunotherapy composition comprises a mixture of 1-2, 1-5, 1-10, 1-20 or 1-40 recombinant delivery vectors, each vector expressing one or more neo-epitopes. In another embodiment, the mixture comprises 1-5, 5-10, 10-15, 15-20, 10-20, 20-30, 30-40, or 40-50 delivery vectors. In another embodiment, a personalized immunotherapy composition comprises a mixture of 1-2, 1-5, 1-10, 1-20 or 1-40 recombinant delivery vectors, each vector expressing one or more neo-epitopes in the context of a fusion protein with a truncated LLO protein, a truncated ActA protein or a PEST amino acid sequence. In one embodiment, the individual delivery vectors present in the mixture of delivery vectors are administered concomitantly to a subject as part of a therapy. In another embodiment, the individual delivery vectors present in the mixture of delivery vectors are administered sequentially to a subject as part of a therapy.

In one embodiment, disclosed herein is an immunogenic mixture of compositions comprising one or more recombinant delivery vectors produced by the process disclosed herein. In another embodiment, each of said delivery vector in said mixture comprises a nucleic acid molecule encoding a fusion polypeptide or chimeric protein comprising one or more neo-epitopes. In another embodiment, each delivery vector in said mixture expresses 1-5, 5-10, 10-15, 15-20, 10-20, 20-30, 30-40, 40-50, 50-60, 60-70, 70-80, 80-90, 90-100, or 100-200 neo-epitopes. In another embodiment, each mixture comprises 1-5, 5-10, 10-15, 15-20, 10-20, 20-30, 30-40, or 40-50 delivery vectors. In another embodiment, the mixture comprises a plurality of delivery vectors, each delivery vector comprising a different set of one or more neo-epitopes. A first set of neo-epitopes can be different from a second set if it includes one neo-epitope that the second set does not. Likewise, a first set of neo-epitopes can be different from a second set if it does not include a neo-epitopes that the second set does include. For example, a first set and a second set of neo-epitopes can include one or more of the same neo-epitopes and can still be different sets, or a first set can be different from a second set of neo-epitopes by virtue of not including any of the same neo-epitopes

In one embodiment, disclosed herein is an immunogenic mixture of compositions comprising one or more recombinant Listeria strains produced by the process disclosed herein. In another embodiment, each of said Listeria in said mixture comprises a nucleic acid molecule encoding a fusion polypeptide or chimeric protein comprising one or more neo-epitopes. In another embodiment, each Listeria in said mixture expresses 1-5, 5-10, 10-15, 15-20, 10-20, 20-30, 30-40, 40-50, 50-60, 60-70, 70-80, 80-90, 90-100, or 100-200 neo-epitopes. In another embodiment, each mixture comprises 1-5, 5-10, 10-15, 15-20, 10-20, 20-30, 30-40, or 40-50 recombinant Listeria strains. In another embodiment, the mixture comprises a plurality of recombinant Listeria strains, each Listeria strain comprising a different set of one or more neo-epitopes. A first set of neo-epitopes can be different from a second set if it includes one neo-epitope that the second set does not. Likewise, a first set of neo-epitopes can be different from a second set if it does not include a neo-epitopes that the second set does include. For example, a first set and a second set of neo-epitopes can include one or more of the same neo-epitopes and can still be different sets, or a first set can be different from a second set of neo-epitopes by virtue of not including any of the same neo-epitopes.

In one embodiment, disclosed herein is a method of eliciting a personalized anti-tumor response in a subject, the method comprising the step of concomitantly or sequentially administering to said subject an immunogenic mixture composition disclosed herein. In another embodiment, disclosed herein is a method of preventing or treating a tumor in a subject, the method comprising the step of concomitantly or sequentially administering to said subject the immunogenic mixture of compositions disclosed herein. In one embodiment, a composition comprising at least one recombinant Listeria strain selected from said mixture of compositions may be administered simultaneously (i.e., in the same medicament), concurrently (i.e., in separate medicaments administered one right after the other in any order) or sequentially in any order with at least another recombinant Listeria strain selected from said mixture of compositions. Sequential administration is particularly useful when a drug substance comprising a recombinant Listeria strain disclosed herein is in different dosage forms (one agent is a tablet or capsule and another agent is a sterile liquid) and/or are administered on different dosing schedules, e.g., one composition from said mixture of compositions comprising one Listeria strain is administered at least daily and another that is administered less frequently, such as once weekly, once every two weeks, or once every three weeks.

In another embodiment, the personalized immunotherapy composition elicits an immune response targeted against one or more neo-epitopes.

In another embodiment, the composition comprises a plurality or combination of Listeria strains, wherein each strain comprises the nucleic acid construct comprising one or more open reading frames encoding one or more peptides comprising at least one unique the neo-epitope.

In another embodiment, the composition comprises a combination of the Listeria strains, wherein the combination comprises a plurality of the neo-epitopes.

It will be appreciated by a skilled artisan that the term “plurality” may encompass an integer above 1. In one embodiment, the term refers to a range of 1-10, 10-20, 20-30, 30-40, 40-50, 60-70, 70-80, 80-90, or 90-100.

In another embodiment, the combination comprises up to about 300 the neo-epitopes.

In another embodiment, the combination comprises a range of about 1-5, 5-10, 10-15, 15-20, 10-20, 20-30, 30-40, 40-50, 50-60, 60-70, 70-80, 80-90, 90-100, or 100-200 neo-epitopes.

In one embodiment, the combination comprises a range of about 8-27 epitopes per vector. In another embodiment, the combination comprises a range of about 21 epitopes per vector. In another embodiment, the combination comprises a range of about 1-5, 1-10, 1-20, 1-30, 1-50, 1-60, 1-70, 1-80, 1-90, 1-100, 1-110, 1-150, 1-200, 1-250, 1-300, or 1-500 epitopes per vector.

In one embodiment all epitopes are neo-epitopes. In another embodiment, at least one epitope per vector is a neo-epitope.

In one embodiment, determination of a number of constructs vs. mutational burden in a delivery vector is performed to determine efficiency of expression and secretion of neo-epitopes. In another embodiment, ranges of linear neo-epitopes are tested, starting with about 50 epitopes per vector. In another embodiment, ranges of linear neo-epitopes are tested, starting with about 1-5, 5-10, 10-20, 20-50, 50-70, 70-90, 90-110, 110-150, 150-200, 200-250, 300-350, or 400-500 epitopes per vector. In one embodiment, constructs include at least one neo-epitope per vector.

In one embodiment, the number of vectors to be used is determined considering the efficiency of translation and secretion of multiple epitopes from a single vector, and the multiplicity of infection (MOI) needed for each Lm vector harboring specific neo-epitopes, or in reference to the number of neo-epitopes.

In one embodiment, the number of vectors to be used (e.g. a Listeria vector) is determined by taking into consideration predefining groups of: known tumor-associated mutations found in circulating tumor cells; known cancer “driver” mutations; and/or known chemotherapy resistance mutations and giving these priority in the 21 amino acid sequence peptide selection (see Example 30). In another embodiment, this can be accomplished by screening identified mutated genes against the COSMIC (Catalogue of somatic mutations in cancer, cancer.Sanger.ac.uk) or Cancer Genome Analysis or other similar cancer-associated gene database. Further, and in another embodiment, screening for immunosuppressive epitopes (T-reg epitopes, IL-10 inducing T helper epitopes, etc.) is utilized to de-select or to avoid immunosuppressive influences on the vector. In another embodiment, selected codons are codon optimized to efficient translation and secretion according to specific the specific delivery vector (e.g. Listeria strain). Example for codons optimized for L. monocytogenes as known in the art is presented in table 8 herein.

In another embodiment, the combination comprises at least two different neo-epitopes amino acid sequences.

In another embodiment, the combination comprises the neo-epitopes in the range of about 1-5, 5-10, 10-15, 15-20, 10-20, 20-30, 30-40, 40-50, 50-60, 60-70, 70-80, 80-90, or 90-100.

In another embodiment, the combination comprises the neo-epitopes in the range of about 50-100.

In another embodiment, the combination comprises up to about 100 the neo-epitopes.

In another embodiment, the combination comprises above about 100 the neo-epitopes.

In another embodiment, the combination comprises up to about 10 the neo-epitopes.

In another embodiment, the combination comprises up to about 20 the neo-epitopes.

In another embodiment, the combination comprises up to about 50 the neo-epitopes.

In another embodiment, the combination comprises about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 the neo-epitopes.

In another embodiment, the combination comprises the neo-epitopes in the range of about 5-15, 5-20, 5-25, 15-20, 15-25, 15-30, 15-35, 20-25, 20-35, 20-45, 30-45, 30-55, 40-55, 40-65, 50-65, 50-75, 60-75, 60-85, 70-85, 70-95, 80-95, 80-105 or 95-105.

In another embodiment, the combination comprises about 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 the neo-epitopes.

In another embodiment, the combination further comprises one or more recombinant attenuated Listeria strain delivery vector comprising a nucleic acid construct comprising one or more open reading frames encoding one or more one or more immunomodulatory molecule(s).

In another embodiment, the immunomodulatory molecule is expressed and secreted from the Listeria strain, wherein the molecule is selected from a group comprising Interferon gamma, a cytokine, a chemokine, a T-cell stimulant, and any combination thereof.

In another embodiment, the combination further comprises one or more recombinant attenuated Listeria strain delivery vector comprising a nucleic acid construct comprising one or more open reading frames encoding one or more peptides comprising one or more epitopes, wherein the epitope(s) comprise immunogenic epitopes present in a disease-bearing tissue or cell of the subject having the disease or condition, wherein administrating the Listeria strain generates a immunotherapy targeted to the subject's disease or condition.

In another embodiment, the composition, as disclosed in any of the above, further comprising an adjuvant.

In another embodiment, the adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

In another embodiment, administering the composition to the subject generates a personalized enhanced anti-disease, or anti-condition immune response in the subject.

In another embodiment, the immune response comprises an anti-cancer or anti-tumor response.

In another embodiment, the immune response comprises an anti-infectious disease response.

In another embodiment, the infectious disease comprises a viral infection.

In another embodiment, the infectious disease comprises a bacterial infection.

In another embodiment, the personalized immunotherapy increases survival time in the subject having the disease or condition.

In another embodiment, the personalized immunotherapy reduces tumor size or metastases size in the subject having the disease or condition.

In another embodiment, the personalized immunotherapy protects against metastases in the subject having the disease or condition.

In another embodiment of the present invention, a DNA immunotherapy comprising the personalized immunotherapy composition as disclosed in any of the above.

In another embodiment of the present invention, a peptide immunotherapy comprising the personalized immunotherapy composition as disclosed in any of the above.

In another embodiment, the immunotherapy further comprises an adjuvant, cytokine, chemokine, or combination thereof.

In another embodiment of this invention, a pharmaceutical composition of the present invention comprising the immunotherapy or personalized immunotherapy composition as disclosed in any of the above and a pharmaceutical carrier.

In another embodiment of this invention, a method of inducing an immune response to at least one neo-epitope present in a disease or condition bearing tissue or cell in a subject having the disease or condition, the method comprising the step of administering the personalized immunotherapy composition or immunotherapy as disclosed in any of the above to the subject.

In another embodiment of this invention, a method of inducing a targeted immune response in a subject having a disease or condition, comprising administering to the subject the immunogenic composition or immunotherapy as disclosed in any of the above, wherein administrating the Listeria strain generates a personalized immunotherapy targeted to the subject's disease or condition.

In another embodiment of this invention, a method of treating, suppressing or inhibiting disease or condition in a subject, the method comprising the step of administrating a personalized immunotherapy composition or immunotherapy as disclosed in any of the above, for targeting the disease or condition.

According to another embodiment of the present invention, a method as described above is disclosed, additionally comprising the step of administrating the composition or immunotherapy orally or parenterally.

In another embodiment administrating parenterally comprises intravenous administration, subcutaneous administration, or intramuscular administration.

In yet another embodiment, the disease or condition is an infectious disease, autoimmune disease, organ transplantation rejection, a tumor or a cancer.

In another embodiment, the tumor or cancer comprises a breast cancer or tumor, a cervical cancer or tumor, an Her2 expressing cancer or tumor, a melanoma, a pancreatic cancer or tumor, an ovarian cancer or tumor, a gastric cancer or tumor, a carcinomatous lesion of the pancreas, a pulmonary adenocarcinoma, a glioblastoma multiforme, a colorectal adenocarcinoma, a pulmonary squamous adenocarcinoma, a gastric adenocarcinoma, an ovarian surface epithelial neoplasm, an oral squamous cell carcinoma, non-small-cell lung carcinoma, an endometrial carcinoma, a bladder cancer or tumor, a head and neck cancer or tumor, a prostate carcinoma, a renal cancer or tumor, a bone cancer or tumor, a blood cancer, or a brain cancer or tumor.

In another embodiment, the infectious disease comprises a viral or bacterial infection.

In another embodiment, the infectious disease is caused by one of the following pathogens: Leishmania, Entamoeba histolytica (which causes amebiasis), Trichuris, BCG/Tuberculosis, Malaria, Plasmodium falciparum, Plasmodium malariae, Plasmodium vivax, Rotavirus, Cholera, Diptheria-Tetanus, Pertussis, Haemophilus influenzae, Hepatitis B, Human papilloma virus, Influenza seasonal), Influenza A (H1N1) Pandemic, Measles and Rubella, Mumps, Meningococcus A+C, Oral Polio Immunotherapies, mono, bi and trivalent, Pneumococcal, Rabies, Tetanus Toxoid, Yellow Fever, Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers, Arenaviruses (LCM, Junin virus, Machupo virus, Guanarito virus, Lassa Fever), Bunyaviruses (Hantaviruses, Rift Valley Fever), Flaviruses (Dengue), Filoviruses (Ebola, Marburg), Burkholderia pseudomallei, Coxiella burnetii (Q fever), Brucella species (brucellosis), Burkholderia mallei (glanders), Chlamydia psittaci (Psittacosis), Ricin toxin (from Ricinus communis), Epsilon toxin of Clostridium perfringens, Staphylococcus enterotoxin B, Typhus fever (Rickettsia prowazekii), other Rickettsias, Food- and Waterborne Pathogens, Bacteria (Diarrheagenic E. coli, Pathogenic Vibrios, Shigella species, Salmonella BCG/, Campylobacter jejuni, Yersinia enterocolitica), Viruses (Caliciviruses, Hepatitis A, West Nile Virus, LaCrosse, Calif. encephalitis, VEE, EEE, WEE, Japanese Encephalitis Virus, Kyasanur Forest Virus, Nipah virus, hantaviruses, Tickborne hemorrhagic fever viruses, Chikungunya virus, Crimean-Congo Hemorrhagic fever virus, Tickborne encephalitis viruses, Hepatitis B virus, Hepatitis C virus, Herpes Simplex virus (HSV), Human immunodeficiency virus (HIV), Human papillomavirus (HPV)), Protozoa (Cryptosporidium parvum, Cyclospora cayatanensis, Giardia lamblia, Entamoeba histolytica, Toxoplasma), Fungi (Microsporidia), Yellow fever, Tuberculosis, including drug-resistant TB, Rabies, Prions, Severe acute respiratory syndrome associated coronavirus (SARS-CoV), Coccidioides posadasii, Coccidioides immitis, Bacterial vaginosis, Chlamydia trachomatis, Cytomegalovirus, Granuloma inguinale, Hemophilus ducreyi, Neisseria gonorrhea, Treponema pallidum, Streptococcus mutans, or Trichomonas vaginalis.

In another embodiment of the present invention, provided is a method of increasing the ratio of T effector cells to regulatory T cells (Tregs) in the spleen and tumor of a subject, wherein the T effector cells are targeted to a neo-epitope present within a disease or condition bearing tissue of a subject, the method comprising the step of administering to the subject personalized immunotherapy composition or immunotherapy as disclosed in any of the above.

In another embodiment of the present invention, provided is a method for increasing antigen-specific T-cells in a subject, wherein the antigen or a peptide fragment thereof comprises one or more neo-epitopes, the method comprising the step of administering to the subject a personalized immunotherapy composition or immunotherapy as disclosed in any of the above.

In another embodiment of the present invention, provided is a method for increasing survival time of a subject having a tumor or suffering from cancer, or suffering from an infectious disease, the method comprising the step of administering to the subject a personalized immunotherapy composition or immunotherapy as disclosed in any of the above.

In another embodiment of the present invention, provided is a method of protecting a subject from a cancer, the method comprising the step of administering to the subject a personalized immunotherapy composition or immunotherapy as disclosed in any of the above.

In another embodiment of the present invention, provided is a method of inhibiting or delaying the onset of cancer in a subject, the method comprising the step of administering to the subject a personalized immunotherapy composition or immunotherapy as disclosed in any of the above.

In another embodiment of the present invention, provided is a method of reducing tumor or metastasis size in a subject, the method comprising the step of administrating to the subject a personalized immunotherapy composition or immunotherapy as disclosed in any of the above.

According to another embodiment of the present invention, the tumor or cancer comprises a breast cancer or tumor, a cervical cancer or tumor, an Her2 expressing cancer or tumor, a melanoma, a pancreatic cancer or tumor, an ovarian cancer or tumor, a gastric cancer or tumor, a carcinomatous lesion of the pancreas, a pulmonary adenocarcinoma, a glioblastoma multiforme, a colorectal adenocarcinoma, a pulmonary squamous adenocarcinoma, a gastric adenocarcinoma, an ovarian surface epithelial neoplasm, an oral squamous cell carcinoma, non-small-cell lung carcinoma, an endometrial carcinoma, a bladder cancer or tumor, a head and neck cancer or tumor, a prostate carcinoma, a renal cancer or tumor, a bone cancer or tumor, a blood cancer, or a brain cancer or tumor.

In another embodiment of the present invention, provided is a method of protecting a subject from an infectious disease, the method comprising the step of administering to the subject a personalized immunotherapy composition or immunotherapy as disclosed in any of the above.

In another embodiment of the present invention, the infectious disease comprises a viral or bacterial infection.

In another embodiment of the present invention, the infectious disease is caused by one of the following pathogens: Leishmania, Entamoeba histolytica (which causes amebiasis), Trichuris, BCG/Tuberculosis, Malaria, Plasmodium falciparum, Plasmodium malariae, Plasmodium vivax, Rotavirus, Cholera, Diptheria-Tetanus, Pertussis, Haemophilus influenzae, Hepatitis B, Human papilloma virus, Influenza seasonal), Influenza A (H1N1) Pandemic, Measles and Rubella, Mumps, Meningococcus A+C, Oral Polio Immunotherapies, mono, bi and trivalent, Pneumococcal, Rabies, Tetanus Toxoid, Yellow Fever, Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers, Arenaviruses (LCM, Junin virus, Machupo virus, Guanarito virus, Lassa Fever), Bunyaviruses (Hantaviruses, Rift Valley Fever), Flaviruses (Dengue), Filoviruses (Ebola, Marburg), Burkholderia pseudomallei, Coxiella burnetii (Q fever), Brucella species (brucellosis), Burkholderia mallei (glanders), Chlamydia psittaci (Psittacosis), Ricin toxin (from Ricinus communis), Epsilon toxin of Clostridium perfringens, Staphylococcus enterotoxin B, Typhus fever (Rickettsia prowazekii), other Rickettsias, Food- and Waterborne Pathogens, Bacteria (Diarrheagenic E. coli, Pathogenic Vibrios, Shigella species, Salmonella BCG/, Campylobacter jejuni, Yersinia enterocolitica), Viruses (Caliciviruses, Hepatitis A, West Nile Virus, LaCrosse, Calif. encephalitis, VEE, EEE, WEE, Japanese Encephalitis Virus, Kyasanur Forest Virus, Nipah virus, hantaviruses, Tickborne hemorrhagic fever viruses, Chikungunya virus, Crimean-Congo Hemorrhagic fever virus, Tickborne encephalitis viruses, Hepatitis B virus, Hepatitis C virus, Herpes Simplex virus (HSV), Human immunodeficiency virus (HIV), Human papillomavirus (HPV)), Protozoa (Cryptosporidium parvum, Cyclospora cayatanensis, Giardia lamblia, Entamoeba histolytica, Toxoplasma), Fungi (Microsporidia), Yellow fever, Tuberculosis, including drug-resistant TB, Rabies, Prions, Severe acute respiratory syndrome associated coronavirus (SARS-CoV), Coccidioides posadasii, Coccidioides immitis, Bacterial vaginosis, Chlamydia trachomatis, Cytomegalovirus, Granuloma inguinale, Hemophilus ducreyi, Neisseria gonorrhea, Treponema pallidum, Streptococcus mutans, or Trichomonas vaginalis.

In another embodiment of the administering results in the generation of a personalized T-cell immune response against the disease or the condition.

According to another embodiment of the present invention, a method as described above is disclosed, additionally comprising the steps of creating the personalized immunotherapy composition, wherein the creating comprises the steps of:
(a) comparing one or more open reading frames (ORFs) in nucleic acid sequences extracted from a disease-bearing biological sample with one or more ORFs in nucleic acid sequences extracted from a healthy biological sample, wherein the comparing identifies one or more nucleic acid sequences encoding one or more peptides comprising one or more neo-epitopes encoded within one or more ORFs from the disease-bearing sample;
(b) transforming an attenuated Listeria strain with a vector comprising a nucleic acid sequence encoding one or more peptides comprising one or more neo-epitopes identified in a.; and, alternatively storing the attenuated recombinant Listeria for administering to the subject at a pre-determined period or administering a composition comprising the attenuated recombinant Listeria strain to the subject, and wherein the administering results in the generation of a personalized T-cell immune response against the disease or the condition; optionally,
(c) obtaining a second biological sample from the subject comprising a T-cell clone or T-infiltrating cell from the T-cell immune response and characterizing specific peptides comprising one or more neo-epitopes bound by MHC Class I or MHC Class II molecules on the T cells, wherein one or more neo-epitopes are immunogenic;
(d) screening for and selecting a nucleic acid construct encoding one or more peptides comprising one or more immunogenic neo-epitope identified in c.; and,
(e) transforming a second attenuated recombinant Listeria strain with a vector comprising a nucleic acid sequence encoding one or more peptides comprising one or more immunogenic neo-epitopes; and, alternatively storing the second attenuated recombinant Listeria for administering to the subject at a pre-determined period or administering a second composition comprising the second attenuated recombinant Listeria strain to the subject,
   wherein the process creates a personalized immunotherapy for the subject.

In one embodiment, the one or more neo-epitopes comprise a plurality of neo-epitopes. Optionally, step (b) can further comprise one or more iterations of randomizing the order of the one or more peptides comprising the plurality of neo-epitopes within the nucleic acid sequence of step (b). Such randomizing can include, for example, randomizing the order of the entire set of one or more peptides comprising the plurality of neo-epitopes, or can comprise randomizing the order of a subset of the one or more peptides comprising a subset of the plurality of neo-epitopes. For example, if the nucleic acid sequence comprises 20 peptides (ordered 1-20) comprising 20 neo-epitopes, the randomizing can comprise randomizing the order of all 20 peptides or can comprise randomizing the order of only a subset of the peptides (e.g., peptides 1-5 or 6-10). Such randomization of the order can facilitate secretion and presentation of the neo-epitopes and of each individual region.

In another embodiment, the step of comparing one or more open reading frames (ORFs) in nucleic acid sequences extracted from a disease-bearing biological sample with one or more ORFs in nucleic acid sequences extracted from a healthy biological sample, further comprises using of a screening assay or screening tool and associated digital software for comparing one or more ORFs in nucleic acid sequences extracted from the disease-bearing biological sample with one or more ORFs in nucleic acid sequences extracted from the healthy biological sample, wherein the associated digital software comprises access to a sequence database that allows screening of mutations within the ORFs in the nucleic acid sequences extracted from the disease-bearing biological sample for identification of immunogenic potential of the neo-epitopes.

In another embodiment of the invention the method as disclosed in any of the above, additionally comprises the step of screening one or more neo-epitopes, peptide comprising one or more neo-epitopes, or both, for hydrophobicity and hydrophilicity.

According to another embodiment of the present invention, a method as described above is disclosed, additionally comprises the step of selecting one or more neo-epitopes, peptides comprising one or more neo-epitopes, or both, that are hydrophilic.

According to another embodiment of the present invention, a method as described above is disclosed, additionally comprises the step of selecting one or more neo-epitopes, peptides comprising one or more neo-epitopes, or both, that are up to 1.6 in the Kyte Doolittle hydropathy plot.

According to another embodiment of the present invention, a method as described above is disclosed, additionally comprising the step of codon optimizing one or more neo-epitopes or peptides comprising one or more neo-epitopes for expression and secretion according to the specific Listeria strain.

According to another embodiment of the present invention, a method as described above is disclosed, additionally comprising the step of screening one or more neo-epitope(s) for immunosuppressive epitopes.

According to another embodiment of the present invention, the biological sample is tissue, cells, blood or sera.

According to another embodiment of the present invention, a method as described above is disclosed, additionally comprises the step of obtaining the disease-bearing biological sample from the subject having the disease or condition.

According to another embodiment of the present invention as disclosed herein, additionally comprises the step of obtaining the healthy biological sample from the subject having the disease or condition.

According to another embodiment, the step of obtaining a second biological sample from the subject comprises obtaining a biological sample comprising T-cell clones or T-infiltrating cells that expand following administration of the second composition comprising the attenuated recombinant Listeria strain.

According to another embodiment of the present invention, a method as described above is disclosed, additionally comprising the steps of: (a) identifying, isolating and expanding T cell clones or T-infiltrating cells that respond against the disease; and, (b) screening for and identifying one or more peptides comprising one or more immunogenic neo-epitopes loaded on specific MHC Class I or MHC Class II molecules to which a T-cell receptor on the T cells binds to.

In another embodiment, the step of screening for and identifying comprises T-cell receptor sequencing, multiplex based flow cytometry, or high-performance liquid chromatography.

In another embodiment, the sequencing comprises using associated digital software and database.

According to another embodiment of the present invention, a method as described above is disclosed, additionally comprising the step of determining the sequencing of the nucleic acid sequences using exome sequencing or transcriptome sequencing.

In one embodiment, a fusion polypeptide or chimeric protein disclosed herein is expressed and secreted by a recombinant Listeria disclosed herein. In another embodiment, the fusion polypeptide, or chimeric protein disclosed herein comprises a C-terminal SIINFEKL-S-6×HIS tag. In another embodiment, the fusion polypeptide, or chimeric protein disclosed herein is expressed and secreted by a recombinant Listeria disclosed herein. In another embodiment, secretion of the antigen, or polypeptides (fusion or chimeric) disclosed herein is detected using a protein, molecule or antibody (or fragment thereof) that specifically binds to a polyhistidine (His) tag. In another embodiment, the fusion polypeptide, or chimeric protein disclosed herein is expressed and secreted by a recombinant Listeria disclosed herein. In another embodiment, secretion of the antigen, or polypeptides (fusion or chimeric) disclosed herein is detected using an antibody, protein or molecule that binds a SIINFEKL-S-6×HIS tag. In another embodiment, the fusion polypeptide of chimeric protein disclosed herein comprise any other tag know in the art, including, but not limited to chitin binding protein (CBP), maltose binding protein (MBP), and glutathione-S-transferase (GST), thioredoxin (TRX) and poly(NANP).

In one embodiment, each neo-epitope is connected with a linker sequence to the following neo-epitope encoded on the same vector. In one embodiment, the linker is 4× glycine DNA sequence. It will be appreciated by a skilled artisan that other linker sequences known in the art may be used in the methods and compositions disclosed herein (see for e.g. Reddy Chichili, V. P., Kumar, V. and Sivaraman, J. (2013), Linkers in the structural biology of protein—protein interactions. Protein Science, 22: 153-167, which is incorporated by reference herein in its entirety). In yet another embodiment the linker is selected from a group comprising SEQ ID NO: 1-11, SEQ ID NO 76-86 accordingly, and any combination thereof.

In one embodiment, the final neo-epitope in an insert is fused to a TAG sequence followed by a stop codon. It will be appreciated by a skilled artisan that a TAG may allow easy detection of the fusion polypeptide or chimeric protein during for example secretion from the Lm vector or when testing construct for affinity to specific T-cells, or presentation by antigen presenting cells.

In one embodiment, about 10 flanking amino acids on each side of the detected mutation are incorporated to accommodate class1 MHC-1 presentation, in order to provide at least some of the different HLA T-cell receptor (TCR) reading frames.

Table 7 herein shows a sample list of 50 neo-epitope peptides wherein each mutation is indicated by a Bolded amino acid letter and is flanked by 10 amino acids on each side providing an 21 amino acid peptide neo-epitope. In one embodiment, if there are more usable 21 amino acid peptides than can fit into a single plasmid, different 21 amino acid peptides are designated into 1^{st}, 2^{nd}, etc. construct by priority rank as needed/desired. In another embodiment, the priority of assignment to one of multiple vectors composing the entire set of desired neo-epitopes are determined based on factors like relative size, priority of transcription, and/or overall hydrophobicity of the translated polypeptide.

In one embodiment different linker sequences are distributed between the neo-epitopes for minimizing repeats. In another embodiment, distributing different linker sequences between the neo-epitopes reduce secondary structures thereby allowing efficient transcription, translation, secretion, maintenance, or stabilization of the plasmid including the insert within the Lm recombinant vector strain population.

In one embodiment, disclosed herein is a process for creating a personalized immunotherapy for a subject having a disease or condition, the process comprising the steps of:
a. comparing one or more open reading frames (ORFs) in nucleic acid sequences extracted from a disease-bearing biological sample with one or more ORFs in nucleic acid sequences extracted from a healthy biological sample, wherein said comparing identifies one or more nucleic acid sequences encoding one or more peptides comprising one or more neo-epitopes encoded within said one or more ORFs from the disease-bearing sample;
b. transforming an attenuated Listeria strain with a vector comprising a nucleic acid sequence encoding one or more peptides comprising said one or more neo-epitopes identified in a.; and, alternatively storing said attenuated recombinant Listeria for administering to said subject at a pre-determined period or administering a composition comprising said attenuated recombinant Listeria strain to said subject, and wherein said administering results in the generation of a personalized T-cell immune response against said disease or said condition; optionally,
c. Obtaining a second biological sample from said subject comprising a T-cell clone or T-infiltrating cell from said T-cell immune response and characterizing specific peptides comprising one or more neo-epitopes bound by MHC Class I or MHC Class II molecules on said T cells, wherein said one or more neo-epitopes are immunogenic;
d. Screening for and selecting a nucleic acid construct encoding one or more peptides comprising one or more immunogenic neo-epitope identified in c.; and,
e. Transforming a second attenuated recombinant Listeria strain with a vector comprising a nucleic acid sequence encoding one or more peptides comprising said one or more immunogenic neo-epitopes; and, alternatively storing said second attenuated recombinant Listeria for administering to said subject at a pre-determined period or administering a second composition comprising said second attenuated recombinant Listeria strain to said subject,
   wherein said process creates a personalized immunotherapy for said subject.

In one embodiment, disclosed herein is a process for creating a personalized immunotherapy for a subject having a disease or condition, the process comprising the steps of:

a. comparing one or more open reading frames (ORFs) in nucleic acid sequences extracted from a disease-bearing biological sample with one or more ORFs in nucleic acid sequences extracted from a healthy biological sample, wherein said comparing identifies one or more nucleic acid sequences encoding one or more peptides comprising one or more neo-epitopes encoded within said one or more ORFs from the disease-bearing sample;
b. transforming a vector with a nucleic acid sequence encoding one or more peptides comprising said one or more neo-epitopes identified in a., or generating a DNA immunotherapy vector or a peptide immunotherapy vector using said nucleic acid sequence encoding one or more peptides comprising said one or more neo-epitopes identified in a.; and, alternatively storing said vector or said DNA immunotherapy or said peptide immunotherapy for administering to said subject at a pre-determined period or administering a composition comprising said vector, said DNA immunotherapy or said peptide immunotherapy to said subject, and wherein said administering results in the generation of a personalized T-cell immune response against said disease or said condition; and optionally,
c. Obtaining a second biological sample from said subject comprising a T-cell clone or T-infiltrating cell from said T-cell immune response and characterizing specific peptides comprising one or more immunogenic neo-epitopes bound by MHC Class I or MHC Class II molecules on said T cells;
d. Screening for and selecting a nucleic acid construct encoding one or more peptides comprising one or more immunogenic neo-epitope identified in c.; and,
e. Transforming a second vector with a nucleic acid sequence comprising one or more open reading frames encoding one or more peptides comprising said one or more immunogenic neo-epitopes or generating a DNA immunotherapy vector or a peptide immunotherapy vector using said nucleic acid sequence encoding one or more peptides comprising said one or more immunogenic neo-epitopes identified in c.; and, alternatively storing said vector or said DNA immunotherapy or said peptide immunotherapy for administering to said subject at a pre-determined period, or administering a composition comprising said vector, said DNA immunotherapy or said peptide immunotherapy to said subject,
wherein said process creates a personalized immunotherapy for said subject.

In one embodiment, disclosed herein is a process for creating a personalized immunotherapy for a subject having a disease or condition, the process comprising the steps of:
a. comparing one or more open reading frames (ORFs) in nucleic acid sequences extracted from a disease-bearing biological sample with one or more ORFs in nucleic acid sequences extracted from a healthy biological sample, wherein said comparing identifies one or more nucleic acid sequences encoding one or more peptides comprising one or more neo-epitopes encoded within said one or more ORFs from the disease-bearing sample;
b. transforming a vector with a nucleic acid sequence encoding one or more peptides comprising said one or more neo-epitopes identified in a., or generating a DNA immunotherapy vector or a peptide immunotherapy vector using said nucleic acid sequence comprising one or more ORFs encoding one or more peptides comprising said one or more neo-epitopes identified in a.; and, alternatively storing said vector or said DNA immunotherapy or said peptide immunotherapy for administering to said subject at a pre-determined period or administering a composition comprising said vector, said DNA immunotherapy or said peptide immunotherapy to said subject, and wherein said administering results in the generation of a personalized T-cell immune response against said disease or said condition; and optionally,
c. Obtaining a second biological sample from said subject comprising a T-cell clone or T-infiltrating cell or blood or tissue specimen whereby response to potential neo-epitope peptides can be identified and selected based on increased or changed T-cell immune response and characterizing by reacting with specific peptides comprising one or more immunogenic neo-epitopes bound by MHC Class I or MHC Class II molecules on said T cells, wherein said one or more neo-epitopes are immunogenic or by PCR based deep sequencing of the T cell receptor specificity and evaluation of increased Tcell responses associated with neo-epitopes;
d. Screening for and selecting a nucleic acid construct encoding one or more peptides comprising one or more immunogenic neo-epitope identified in c.; and,
e. Transforming a second vector with a nucleic acid sequence encoding one or more peptides comprising said one or more immunogenic neo-epitopes, or generating a DNA immunotherapy vector or a peptide immunotherapy vector using said nucleic acid sequence encoding one or more peptides comprising said one or more immunogenic neo-epitopes identified in c.; and, alternatively storing said vector or said DNA immunotherapy or said peptide immunotherapy for administering to said subject at a pre-determined period, or administering a composition comprising said vector, said DNA immunotherapy or said peptide immunotherapy to said subject,
   wherein said process creates a personalized immunotherapy for said subject.

In another embodiment, provided herein is a system for providing a personalized immunotherapy for a subject having a disease or condition, comprising the following components:
g. a disease-bearing biological sample obtained from said subject having said disease or condition;
h. a healthy biological sample, wherein said healthy biological sample is obtained from said human subject having said disease or condition or another healthy human subject;
i. a screening assay or screening tool and associated digital software for comparing one or more open reading frames (ORFs) in nucleic acid sequences extracted from said disease-bearing biological sample with open reading frames in nucleic acid sequences extracted from said healthy biological sample, and for identifying mutations in said ORFs encoded by said nucleic acid sequences of said disease-bearing sample, wherein said mutations comprise one or more neo-epitopes;
   i. wherein said associated digital software comprises access to a sequence database that allows screening of said mutations within said ORFs for identification of T-cell epitope(s) or immunogenic potential, or any combination thereof;
j. a nucleic acid cloning and expression kit for cloning and expressing a nucleic acid encoding one or more peptides comprising said one or more neo-epitopes from said disease-bearing sample;
k. an immunogenic assay for testing the T-cell immunogenecity and/or binding of candidate peptides comprising one or more neo-epitopes;
l. analytic equipment, and associated software for sequencing and analyzing nucleic acid sequences, peptide amino acid sequences and T-cell receptor amino acid sequences.
m. an attenuated Listeria delivery vector for transforming with a plasmid vector comprising a nucleic acid construct comprising one or more open reading frames encoding said identified immunogenic peptides comprising one or more immunogenic neo-epitopes of step (e),
   i. wherein once transformed, said Listeria is stored or is administered to said human subject in (a) as part of an immunogenic composition; or
n. a delivery vector; and optionally
o. a vector for transforming said delivery vector, said vector comprising a nucleic acid construct comprising one or more open reading frames encoding one or more peptides comprising one or more neo-epitopes, wherein said neo-epitope(s) comprise immunogenic epitopes present in a disease-bearing tissue or cell of said subject having said disease or condition.

In another embodiment, said one or more peptides are encoded by one or more open reading frames (ORFs) in said nucleic acid sequence.

In another embodiment, a disease is an infectious disease, or a tumor or cancer.

In another embodiment, said delivery vector comprises a bacterial delivery vector. In another related aspect said delivery vector comprises a viral vector delivery vector. In another related aspect said delivery vector comprises a peptide immunotherapy delivery vector. In another related aspect, said delivery vector comprises a DNA immunotherapy delivery vector.

In one embodiment, provided herein is a process for creating a personalized immunotherapy, the process comprising the steps of:
a. obtaining a disease-bearing biological sample from a subject having said disease or condition;
b. extracting nucleic acids from said disease-bearing sample;
c. obtaining a healthy biological sample from said subject in step (a) or from a different individual of the same species;
d. extracting nucleic acids from said healthy sample;
e. sequencing the extracted nucleic acid from steps (b) and (d);
f. comparing one or more open reading frames (ORFs) in nucleic acid sequences extracted from said disease-bearing biological sample with open reading frames in nucleic acid sequences extracted from said healthy biological sample, and for identifying mutated nucleic acid sequences within said ORFs of said disease-bearing sample, wherein said ORFs encodes a peptide comprising one or more neo-epitopes;
g. identifying mutated sequences within said ORFs in said disease-bearing sample, wherein said ORFs encodes a peptide comprising one or more neo-epitopes;
a. wherein said neo-epitopes are identified using methods well known in the art, including, but not limited to T-cell receptor (TCR) sequencing, or whole exome sequencing.
h. expressing said one or more peptides comprising said identified mutated nucleic acid sequences;
i. screening each peptide comprising said one or more neo-epitopes for an immunogenic T-cell response, wherein the presence of an immunogenic T-cell response correlates with presence of one or more neo-epitopes comprising a T-cell epitope;
j. identifying and selecting a nucleic acid sequence that encodes a one or more immunogenic peptides comprising one or more immunogenic neo-epitopes that are T-cell epitopes, and transforming an attenuated Listeria strain with a plasmid vector comprising said sequence;
k. culturing and characterizing said attenuated Listeria strain to confirm expression and secretion of said one or more immunogenic peptides; and,
l. storing said attenuated Listeria for administering to said subject at a pre-determined period or administering said attenuated Listeria strain to said subject, wherein said attenuated Listeria strain is administered as part of an immunogenic composition.

In another embodiment, the process of obtaining a second biological sample from said subject comprises obtaining a biological sample comprising T-cell clones or T-infiltrating cells that expand following administration of said second composition comprising said attenuated recombinant Listeria strain.

In another embodiment, the process of characterizing specific peptides comprising one or more immunogenic neo-epitopes bound by MHC Class I or MHC Class II molecules on said T cells comprises the steps of:
a. Identifying, isolating and expanding T cell clones or T-infiltrating cells that respond against said disease;
b. Screening for and identifying one or more peptides comprising one or more immunogenic neo-epitopes loaded on specific MHC Class I or MHC Class II molecules to which a T-cell receptor on said T cells binds to.

In another embodiment, a screening step for and identifying one or more peptides comprising one or more immunogenic neo-epitopes loaded on specific MHC Class I or MHC Class II molecules comprises contacting said T-cells with said one or more peptides. In another embodiment, said screening step for and identifying comprises performing T-cell receptor sequencing, multiplex based flow cytometry, or high-performance liquid chromatography to determine peptide specificity. It will be well appreciated by a skilled artisan that methods for determining peptides that bind to T-cell receptors are well known in the art.

In one embodiment, the step of comparing in a system or a process of creating a personalized immunotherapy provided herein, comprises a use of a screening assay or screening tool and associated digital software for comparing one or more open reading frames (ORFs) in nucleic acid sequences extracted from said disease-bearing biological sample with open reading frames in nucleic acid sequences extracted from said healthy biological sample, and for identifying mutated nucleic acid sequences within said ORFs of said disease-bearing sample that encode or are comprised within a peptide comprising one or more neo-epitopes. In another embodiment, the associated digital software comprises access to a sequence database that allows screening of said disease-bearing nucleic acid sequences within said ORFs or the corresponding digitally translated amino acid sequence encoding said peptide comprising one or more neo-epitopes for identification of a T-cell epitope or immunogenic potential, or any combination thereof.

In one embodiment, a step of screening for an immunogenic T-cell response in the system or process of creating a personalized immunotherapy provided comprises use of an immune response assay well known in the art, including for example T-cell proliferation assays, in vitro tumor regression assays using T-cells activated with said neo-epitope and co-incubated with tumor cells using a ⁵¹Cr-release assay or a ³H-thymidine assay, an ELISA assay, an ELlspot assay, and a FACS analysis. (See for example U.S. Pat. No. 8,771,702, which is incorporated herein in its entirety).

In another embodiment, the bacterial sequence is a Listerial sequence, wherein in some embodiments, said Listeria sequence is an hly signal sequence or an actA signal sequence.

In another embodiment, the disease is a localized disease. In another embodiment, the disease is a tumor or cancer. In another embodiment, the tumor or cancer is a solid tumor or cancer. In another embodiment, the tumor or cancer is a liquid tumor or cancer. In another embodiment, an abnormal or unhealthy biological sample comprises a tumor, or a cancer, or a portion thereof.

In one embodiment, the disease is an infectious disease. In another embodiment, the infectious disease is an infectious viral disease or an infectious bacterial disease. In another embodiment, a neo-epitope identified by the process provided herein is an infectious disease-associated-specific epitope.

In another embodiment, a neo-epitope comprises a unique tumor or cancer neo-epitope. In another embodiment, a neo-epitope comprises a cancer-specific or tumor-specific epitope. In another embodiment, a neo-epitope is immunogenic. In another embodiment, a neo-epitope is recognized by T-cells. In another embodiment, a peptide comprising one or more neo-epitopes activates a T-cell response against a tumor or cancer, wherein said response is personalized to said subject.

In another embodiment, a neo-epitope comprises a unique tumor or cancer neo-epitope. In another embodiment, a neo-epitope comprises a unique epitope related to an infectious disease. In one embodiment, the infectious disease epitope directly correlates with the disease. In an alternate embodiment, the infectious disease epitope is associated with the infectious disease.

In another embodiment, the process provided herein allows the generation of a personalized enhanced anti-disease, or anti-infection, or anti-infectious disease, or anti-tumor immune response in said subject having a disease. In another embodiment, the process provided herein allows personalized treatment or prevention of said disease, or said infection or infectious disease, or said tumor or cancer in a subject. In another embodiment, the process provided herein increases survival time in said subject having said disease, or said infection or infectious disease, or said tumor or cancer.

In one embodiment, the present invention provides an immunogenic composition comprising a recombinant Listeria strain provided herein, and a pharmaceutically acceptable carrier. In another embodiment, provided herein are one or more immunogenic compositions comprising one or more recombinant Listeria strains, wherein each Listeria strain expresses one or more different peptides comprising one or more different neo-epitopes. In another embodiment each Listeria expresses a range of neo-epitopes. In another embodiment, each peptide comprises one or more neo-epitopes that are T-cell epitopes. In one embodiment, provided herein is a method of eliciting targeted, personalized anti-tumor T cell response in a subject, the method comprising the step of administering to the subject an effective amount of an immunogenic composition comprising a recombinant Listeria strain provided herein, wherein the Listeria strain expresses one or more neo-epitopes. In another embodiment, a Listeria strain comprises one of the following: a nucleic acid molecule comprising a first open reading frame encoding a fusion polypeptide, wherein the fusion polypeptide comprises an immunogenic polypeptide or fragment thereof fused to a peptide comprising one or more neo-epitopes associated with cancer disease; or, a minigene nucleic acid construct comprising a first open reading frame encoding a chimeric protein, wherein said chimeric protein comprises a Listerial secretion signal sequence, an ubiquitin (Ub) protein, and one or more peptides each comprising one or more neo-epitopes associated with a tumor or a cancer, wherein said signal sequence, said ubiquitin and said one or more peptides are respectively arranged in tandem, or are operatively linked, from the amino terminus to the carboxy terminus.

In another embodiment, the fusion peptides are further linked to a HIS tag or a SIINFEKL tag. In another embodiment, the tag sequence comprises a C-terminal SIINFEKL and 6 His amino acids. In another embodiment, the tag sequence is an amino acid or nucleic acid sequence that allows for easy detection of the neo-epitope. In another embodiment, the tag sequence is an amino acid or nucleic acid sequence that for confirmation of secretion of a neo-epitope disclosed herein. It will be appreciated by a skilled artisan that the sequences for the tags may be incorporated into the fusion peptide sequences on the plasmid or phage vector. These tags may be expressed and the antigenic epitopes presented allowing a clinician to follow the immunogenicity of the secreted peptide by following immune responses to these “tag” sequence peptides. Such immune response can be monitored using a number of reagents including but not limited to, monoclonal antibodies and DNA or RNA probes specific for these tags.

In another embodiment, a method of this invention is increasing the ratio of T effector cells to regulatory T cells (Tregs) in the spleen and tumor of a subject, wherein said T effector cells are targeted to a neo-epitope present within abnormal or unhealthy tissue of a subject, for example a tumor tissue or a cancer, the method comprising the step of administering to the subject an immunogenic composition comprising a recombinant Listeria strain provided herein.

In another embodiment, a method of this invention is for increasing antigen-specific T-cells in a subject, wherein said antigen or a peptide fragment thereof comprises one or more neo-epitopes, the method comprising the step of administering to the subject an immunogenic composition comprising a recombinant Listeria strain provided herein.

In another embodiment, a method of this invention is for increasing survival time of a subject having a tumor or suffering from cancer, or suffering from an infectious disease, the method comprising the step of administering to the subject an immunogenic composition comprising a recombinant Listeria strain provided herein.

In another embodiment, a method of this invention is treating a tumor or a cancer or an infection or an infectious disease in a subject, the method comprising the step of administering to the subject an immunogenic composition comprising a recombinant Listeria strain provided herein.

### I. Personalizing Immunotherapy

In one embodiment, a process of this invention creates a personalized immunotherapy. In another embodiment, a process of creating a personalized immunotherapy for a subject having a disease or condition comprises identifying and selecting neo-epitopes within mutated and variant antigens (neo-antigens) that are specific to said patient's disease. In another embodiment, a process for creating a personalized immunotherapy for a subject is in order to provide a treatment for said subject. In another embodiment, personalized immunotherapy may be used to treat such diseases as cancer, autoimmune disease, organ transplantation rejection, bacterial infection, viral infection, and chronic viral illnesses such as HIV.

A step in a process of creating a personalized immunotherapy is, in one embodiment, to obtain an abnormal or unhealthy biological sample, from a subject having a disease or condition. As used herein, the term “abnormal or unhealthy biological sample” is used interchangeably with “disease-bearing biological sample” or “disease-bearing sample” having all the same meanings and qualities. In one embodiment, a biological sample is a tissue, cells, blood, any sample obtained from a subject that comprises lymphocytes, any sample obtained from a subject that comprises disease-bearing cells, or any sample obtained from a subject that is healthy but is also comparable to a disease-bearing sample that is obtained from the same subject or similar individual.

In one embodiment, an abnormal or unhealthy biological sample comprises a tumor tissue or a cancer tissue or a portion thereof. In another embodiment, a tumor or cancer may be a solid tumor. In another embodiment, a tumor or cancer is not a solid tumor or cancer, for example a blood cancer or a breast cancer wherein a tumor does not form.

In another embodiment, a tumor sample relates to any sample such as a bodily sample derived from a patient containing or being expected of containing tumor or cancer cells. The bodily sample may be any tissue sample such as blood, a tissue sample obtained from the primary tumor or from tumor metastases or any other sample containing tumor or cancer cells. In yet another embodiment, a bodily sample is blood, cells from saliva, or cells from cerebrospinal fluid. In another embodiment, a tumor sample relates to one or more isolated tumor or cancer cells such as circulating tumor cells (CTCs) or a sample containing one or more isolated tumor or cancer cells such as circulating tumor cells (CTCs). In another embodiment, a tumor or a cancer comprises a breast cancer or tumor. In another embodiment, a tumor or a cancer comprises is a cervical cancer or tumor. In another embodiment, a tumor or a cancer comprises a Her2 containing tumor or cancer. In another embodiment, a tumor or a cancer comprises melanoma tumor or cancer. In another embodiment, a tumor or a cancer comprises a pancreatic tumor or cancer. In another embodiment, a tumor or a cancer comprises an ovarian tumor or cancer. In another embodiment, a tumor or a cancer comprises a gastric tumor or cancer. In another embodiment, a tumor or a cancer comprises a carcinomatous lesion of the pancreas. In another embodiment, a tumor or a cancer comprises a pulmonary adenocarcinoma tumor or cancer. In another embodiment, a tumor or a cancer comprises a glioblastoma multiforme tumor or cancer. In another embodiment, a tumor or a cancer comprises a colorectal adenocarcinoma tumor or cancer. In another embodiment, a tumor or a cancer comprises a pulmonary squamous adenocarcinoma tumor or cancer. In another embodiment, a tumor or a cancer comprises a gastric adenocarcinoma tumor or cancer. In another embodiment, a tumor or a cancer comprises an ovarian surface epithelial neoplasm (e.g. a benign, proliferative or malignant variety thereof) tumor or cancer. In another embodiment, a tumor or a cancer comprises an oral squamous cell carcinoma tumor or cancer. In another embodiment, a tumor or a cancer comprises a non-small-cell lung carcinoma tumor or cancer. In another embodiment, a tumor or a cancer comprises an endometrial carcinoma tumor or cancer. In another embodiment, a tumor or a cancer comprises a bladder tumor or cancer. In another embodiment, a tumor or a cancer comprises a head and neck tumor or cancer. In another embodiment, a tumor or a cancer comprises a prostate carcinoma tumor or cancer. In another embodiment, a tumor or a cancer comprises a gastric adenocarcinoma tumor or cancer. In another embodiment, a tumor or a cancer comprises an oropharyngeal tumor or cancer. In another embodiment, a tumor or a cancer comprises a lung tumor or cancer. In another embodiment, a tumor or a cancer comprises an anal tumor or cancer. In another embodiment, a tumor or a cancer comprises a colorectal tumor or cancer. In another embodiment, a tumor or a cancer comprises an esophageal tumor or cancer. In another embodiment, a tumor or a cancer comprises a mesothelioma tumor or cancer.

In another embodiment, an abnormal or unhealthy biological sample comprises non-tumor or cancerous tissue. In another embodiment, an abnormal or unhealthy biological sample comprises cells isolated from a blood sample, cells from saliva, or cells from cerebral spinal fluid. In another embodiment, an abnormal or unhealthy biological sample comprises a sample of any tissue or portion thereof that is considered abnormal or unhealthy.

In one embodiment, other non-tumor or non-cancerous diseases, including infectious diseases from which a disease-bearing biological sample can be obtained for analysis according to the process provided herein, are encompassed by the present invention. In another embodiment, an infectious disease comprises a viral infection. In another embodiment, an infectious disease comprises a chronic viral infection. In another embodiment, an infectious disease comprises a chronic viral illness such as HIV. In another embodiment, an infectious disease comprises a bacterial infection. In another embodiment, the infectious disease is a parasitic infection.

In one embodiment, the infectious disease is one caused by, but not limited to, any one of the following pathogens: Leishmania, Entamoeba histolytica (which causes amebiasis), Trichuris, BCG/Tuberculosis, Malaria, Plasmodium falciparum, Plasmodium malariae, Plasmodium vivax, Rotavirus, Cholera, Diptheria-Tetanus, Pertussis, Haemophilus influenzae, Hepatitis B, Human papilloma virus, Influenza seasonal), Influenza A (H1N1) Pandemic, Measles and Rubella, Mumps, Meningococcus A+C, Oral Polio Immunotherapies, mono, bi and trivalent, Pneumococcal, Rabies, Tetanus Toxoid, Yellow Fever, Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers, Arenaviruses (LCM, Junin virus, Machupo virus, Guanarito virus, Lassa Fever), Bunyaviruses (Hantaviruses, Rift Valley Fever), Flaviruses (Dengue), Filoviruses (Ebola, Marburg), Burkholderia pseudomallei, Coxiella burnetii (Q fever), Brucella species (brucellosis), Burkholderia mallei (glanders), Chlamydia psittaci (Psittacosis), Ricin toxin (from Ricinus communis), Epsilon toxin of Clostridium perfringens, Staphylococcus enterotoxin B, Typhus fever (Rickettsia prowazekii), other Rickettsias, Food- and Waterborne Pathogens, Bacteria (Diarrheagenic E. coli, Pathogenic Vibrios, Shigella species, Salmonella BCG/, Campylobacter jejuni, Yersinia enterocolitica), Viruses (Caliciviruses, Hepatitis A, West Nile Virus, LaCrosse, Calif. encephalitis, VEE, EEE, WEE, Japanese Encephalitis Virus, Kyasanur Forest Virus, Nipah virus, hantaviruses, Tickborne hemorrhagic fever viruses, Chikungunya virus, Crimean-Congo Hemorrhagic fever virus, Tickborne encephalitis viruses, Hepatitis B virus, Hepatitis C virus, Herpes Simplex virus (HSV), Human immunodeficiency virus (HIV), Human papillomavirus (HPV)), Protozoa (Cryptosporidium parvum, Cyclospora cayatanensis, Giardia lamblia, Entamoeba histolytica, Toxoplasma), Fungi (Microsporidia), Yellow fever, Tuberculosis, including drug-resistant TB, Rabies, Prions, Severe acute respiratory syndrome associated coronavirus (SARS-CoV), Coccidioides posadasii, Coccidioides immitis, Bacterial vaginosis, Chlamydia trachomatis, Cytomegalovirus, Granuloma inguinale, Hemophilus ducreyi, Neisseria gonorrhea, Treponema pallidum, Trichomonas vaginalis, or any other infectious disease known in the art that is not listed herein.

In one embodiment, pathogenic protozoans and helminths infections include: amebiasis; malaria; leishmaniasis; trypanosomiasis; toxoplasmosis; Pneumocystis carinii; babesiosis; giardiasis; trichinosis; filariasis; schistosomiasis; nematodes; trematodes or flukes; and cestode (tapeworm) infections.

In another embodiment, the infectious disease is a livestock infectious disease. In another embodiment, livestock diseases can be transmitted to man and are called “zoonotic diseases.” In another embodiment, these diseases include, but are not limited to, Foot and mouth disease, West Nile Virus, rabies, canine parvovirus, feline leukemia virus, equine influenza virus, infectious bovine rhinotracheitis (IBR), pseudorabies, classical swine fever (CSF), IBR, caused by bovine herpesvirus type 1 (BHV-1) infection of cattle, and pseudorabies (Aujeszky's disease) in pigs, toxoplasmosis, anthrax, vesicular stomatitis virus, Rhodococcus equi, Tularemia, Plague (Yersinia pestis), Trichomonas.

In one embodiment, other non-tumor or non-cancerous diseases, including autoimmune diseases from which a disease-bearing biological sample can be obtained for analysis according to the process provided herein, are encompassed by the present invention. It will be appreciated by the skilled artisan that the term “autoimmune disease” refers to a disease or condition arising from immune reactions directed against an individual's own tissues, organs or manifestation thereof or resulting condition therefrom. As used herein the term “autoimmune disease” includes cancers and other disease states where the antibodies that are directed towards self-tissues are not necessarily involved in the disease condition but are still important in diagnostics. Further, in one embodiment, it refers to a condition that results from, or is aggravated by, the production of autoantibodies by B cells of antibodies that are reactive with normal body tissues and antigens. In other embodiments, the autoimmune disease is one that involves secretion of an autoantibody that is specific for an epitope from a self-antigen (e.g. a nuclear antigen).

In an effort to treat a subject having an autoimmune disease, in one embodiment, this invention comprises systems and methods to identify auto-reactive neo-epitopes, wherein said system or process comprises methods to immunize a subject having an autoimmune disease against these auto-reactive neo-epitopes, in order to induce tolerance mediated by antibodies or immunosuppressor cells, for examples Tregs or MDSCs.

In one embodiment, an autoimmune disease comprises a systemic autoimmune disease. The term “systemic autoimmune disease” refers to a disease, disorder or a combination of symptoms caused by autoimmune reactions affecting more than one organ. In another embodiment, a systemic autoimmune disease includes, but is not limited to, Anti-GBM nephritis (Goodpasture's disease), Granulomatosis with polyangiitis (GPA), microscopic polyangiitis (MP A), systemic lupus erythematosus (SLE), polymyositis (PM) or Celiac disease.

In one embodiment, an autoimmune disease comprises a connective tissue disease. The term “connective tissue disease” refers to a disease, condition or a combination of symptoms caused by autoimmune reactions affecting the connective tissue of the body. In another embodiment, a connective tissue disease includes, but is not limited to, systemic lupus erythematosus (SLE), polymyositis (PM), systemic sclerosis or mixed connective tissue disease (MCTD).

In one embodiment, other non-tumor or non-cancerous diseases, including organ transplantation rejection from which a disease-bearing biological sample can be obtained for analysis according to the process provided herein, are encompassed by the present invention. In another embodiment, the rejected organ is a solid organ, including but not limited to a heart, a lung, a kidney, a liver, pancreas, intestine, stomach, testis, cornea, skin, heart valve, a blood vessel, or bone. In another embodiment, the rejected organs include but are not limited to a blood tissue, bone marrow, or islets of Langerhans cells.

In an effort to treat a transplant subject having a rejection of the transplanted organ or is experiencing graft v. host disease (GVhD), in one embodiment, this invention comprises systems and methods to identify auto-reactive neo-epitopes, wherein said system or process comprises methods to immunize a subject having an autoimmune disease against these auto-reactive neo-epitopes, in order to induce tolerance mediated by antibodies or immunosuppressor cells, for examples Tregs or MDSCs.

Samples may be obtained using routine biopsy procedures well known in the art. Biopsies may comprise the removal of cells or tissues from a subject by skilled medical personnel, for example a pathologist. There are many different types of biopsy procedures. The most common types include: (1) incisional biopsy, in which only a sample of tissue is removed; (2) excisional biopsy, in which an entire lump or suspicious area is removed; and (3) needle biopsy, in which a sample of tissue or fluid is removed with a needle. When a wide needle is used, the procedure is called a core biopsy. When a thin needle is used, the procedure is called a fine-needle aspiration biopsy.

In one embodiment, a sample of this invention is obtained by incisional biopsy. In another embodiment a sample is obtained by an excisional biopsy. In another embodiment, a sample is obtained using a needle biopsy. In another embodiment, a needle biopsy is a core biopsy. In another embodiment, a biopsy is a fine-needle aspiration biopsy. In another embodiment, a sample is obtained from as part of a blood sample. In another embodiment, a sample is obtained as part of a cheek swab. In another embodiment, a sample is obtained as part of a saliva sampling. In another embodiment, a biological sample comprises all or part of a tissue biopsy. In another embodiment, a tissue biopsy is taken and cells from that tissue sample are collected, wherein the cells comprise a biological sample of this invention. In another embodiment, a sample of this invention is obtained as part of a cell biopsy. In another embodiment, multiple biopsies may be taken from the same subject. In another embodiment, biopsies from the same subject may be collected from the same tissue or cells. In another embodiment, biopsies from the same subject may be collected from a different tissue of cell source within the subject.

In one embodiment, a biopsy comprises a bone marrow tissue. In another embodiment, a biopsy comprises a blood sample. In another embodiment, a biopsy comprises a biopsy of gastrointestinal tissue, for example esophagus, stomach, duodenum, rectum, colon and terminal ileum. In another embodiment, a biopsy comprises lung tissue. In another embodiment, a biopsy comprises prostate tissue. In another embodiment, a biopsy comprises liver tissue. In another embodiment, a biopsy comprises nervous system tissue, for example a brain biopsy, a nerve biopsy, or a meningeal biopsy. In another embodiment, a biopsy comprises urogenital tissue, for example a renal biopsy, an endometrial biopsy or a cervical conization. In another embodiment, a biopsy comprises a breast biopsy. In another embodiment, a biopsy comprises a lymph node biopsy. In another embodiment, a biopsy comprises a muscle biopsy. In yet another embodiment, a biopsy comprises a skin biopsy. In another embodiment, a biopsy comprises a bone biopsy. In another embodiment, a disease-bearing sample pathology of each sample is examined to confirm a diagnosis of the diseased tissue. In another embodiment, a healthy sample is examined to confirm a diagnosis of the health tissue.

In one embodiment, normal or a healthy biological sample is obtained from the subject. In another embodiment, the normal or healthy biological sample is a non-tumor sample which relates to any sample such as a bodily sample derived from a subject. The sample may be any tissue sample such as healthy cells obtained from a biological sample provided herein. In another embodiment, the normal or healthy biological sample is obtained from another individual which in one embodiment, is a related individual. In another embodiment, another individual is of the same species as the subject. In another embodiment, another individual is a healthy individual not containing or not being expected of containing a disease-bearing biological sample. In another embodiment, another individual is a healthy individual not containing or not being expected of containing tumor or cancer cells. It will be appreciated by a skilled artisan that the healthy individual may be screened using methods known in the art for the presence of a disease in order to determine that he or she is healthy. A disease-bearing biological sample and a healthy biological sample can both be obtained from the same tissue (e.g., a tissue section containing both tumor tissue and surrounding normal tissue). Preferably, healthy biological samples consist essentially or entirely of normal, healthy cells and can be used in comparison to a disease-bearing biological sample (e.g., a sample thought to comprise cancer cells or a particular type of cancer cells). Preferably, the samples are of the same type (e.g., both blood or both sera). For example, if the disease-bearing biological sample comprises cells, preferably the cells in the healthy biological sample have the same tissue origin as the disease-bearing cells in the disease-bearing biological sample (e.g., lung or brain) and arise from the same cell type (e.g., neuronal, epithelial, mesenchymal, hematopoietic).

In another embodiment, the normal or healthy biological sample is obtained at the same time. The terms “normal or healthy biological sample” and “reference sample” or “reference tissue” are used interchangeably throughout, having all the same meanings and qualities. In another embodiment, a “reference” may be used to correlate and compare the results obtained in from a tumor specimen. In another embodiment, a “reference” can be determined empirically by testing a sufficiently large number of normal specimens from the same species. In another embodiment, the normal or healthy biological sample is obtained at a different time, wherein the time may be such that the normal of healthy sample is obtained prior to obtaining the abnormal or healthy sample or afterwards. Methods of obtaining comprise those used routinely in the art for biopsy or blood collection. In another embodiment, a sample is a frozen sample. In another embodiment, a sample is comprised as a tissues paraffin embedded (FFPE) tissue block.

In one embodiment, following obtaining said normal or healthy biological sample, said sample is processed for extracting nucleic acids using techniques and methodologies well known in the art. In another embodiment, nucleic acids extracted comprise DNA. In another embodiment, nucleic acids extracted comprise RNA. In another embodiment, RNA is mRNA.

In another embodiment, a next generation sequencing (NGS) library is prepared. Next-generation sequencing libraries may be constructed and may undergo exome or targeted gene capture. In another embodiment, a cDNA expression library is made using techniques known in the art, for example see US20140141992, which is hereby incorporated in full.

A process of this invention for creating a personalized immunotherapy may comprise use of the extracted nucleic acid from the abnormal or unhealthy sample and the extracted nucleic acid from the normal or healthy reference sample in order to identify somatic mutations or sequence differences present in the abnormal or unhealthy sample as compared with the normal or healthy sample, wherein these sequence having somatic mutations or differences encode an expressed amino acid sequence. In one embodiment, a peptide expressing said somatic mutations or sequence differences, may in certain embodiments, be referred to throughout as “neo-epitopes.”

It will be appreciated by a skilled artisan that the term “neo-epitope” may also refer to an epitope that is not present in a reference sample, such as a normal non-cancerous or germline cell or tissue but is found in disease-bearing tissues, for example in a cancer cell. This includes, in another embodiment, situations wherein in a normal non-cancerous or germline cell a corresponding epitope is found, however, due to one or more mutations in a cancer cell the sequence of the epitope is changed so as to result in the neo-epitope. In another embodiment, a neo-epitope comprises a mutated epitope. In another embodiment, a neo-epitope has non-mutated sequence on either side of the epitope. In one embodiment, a neo-epitope is a linear epitope. In another embodiment, a neo-epitope is considered solvent-exposed and therefore accessible to T-cell antigen receptors.

In another embodiment, one or more peptides provided herein do not comprise one or more immunosuppressive T-regulatory neo-epitopes. In another embodiment, a neo-epitope identified and used by the methods provided herein does not comprise an immunosuppressive epitope. In another embodiment, a neo-epitope identified and used by the methods provided herein does not activate T-regulatory (T-reg) cells.

In another embodiment, a neo-epitope is immunogenic. In another embodiment, a neo-epitope comprises a T-cell epitope. In another embodiment, a neo-epitope comprises an adaptive immune response epitope.

In another embodiment, a neo-epitope comprises a single mutation. In another embodiment, a neo-epitope comprises at least 2 mutations. In another embodiment, a neo-epitope comprises at least 2 mutations. In another embodiment, a neo-epitope comprises at least 3 mutations. In another embodiment, a neo-epitope comprises at least 4 mutations. In another embodiment, a neo-epitope comprises at least 5 mutations. In another embodiment, a neo-epitope comprises at least 6 mutations. In another embodiment, a neo-epitope comprises at least 7 mutations. In another embodiment, a neo-epitope comprises at least 8 mutations. In another embodiment, a neo-epitope comprises at least 9 mutations. In another embodiment, a neo-epitope comprises at least 10 mutations. In another embodiment, a neo-epitope comprises at least 20 mutations. In another embodiment, a neo-epitope comprises 1-10, 11-20, 20-30, and 31-40 mutations.

In another embodiment, a neo-epitope is associated with said disease or condition of said subject. In another embodiment, a neo-epitope is causative of said disease or condition of said subject. In another embodiment, a neo-epitope is present within said disease bearing biological sample. In another embodiment, a neo-epitope is present within said disease bearing biological tissue but is not causative or associated with said disease or condition.

In another embodiment, a peptide, a polypeptide or a fusion peptide of this invention comprises one neo-epitope. In another embodiment, a peptide, a polypeptide or a fusion peptide of this invention comprises two neo-epitopes. In another embodiment, a peptide, a polypeptide or a fusion peptide of this invention comprises 3 neo-epitopes. In another embodiment, a peptide, a polypeptide or a fusion peptide of this invention comprises 4 neo-epitopes. In another embodiment, a peptide, a polypeptide or a fusion peptide of this invention comprises 5 neo-epitopes. In another embodiment, a peptide, a polypeptide or a fusion peptide of this invention comprises 6 neo-epitopes. In another embodiment, a peptide, a polypeptide or a fusion peptide of this invention comprises 7 neo-epitopes. In another embodiment, a peptide, a polypeptide or a fusion peptide of this invention comprises 8 neo-epitopes. In another embodiment, a peptide, a polypeptide or a fusion peptide of this invention comprises 9 neo-epitopes. In another embodiment, a peptide, a polypeptide or a fusion peptide of this invention comprises 10 or more neo-epitopes.

In one embodiment, a step towards identifying neo-epitopes comprises sequencing the extracted nucleic acids obtained from the abnormal or unhealthy biological sample and sequencing the extracted nucleic acids obtained from the normal or healthy biological reference sample. In another embodiment, the entire genome is sequenced. In another embodiment, the exome is sequenced. In yet another embodiment, the transcriptome is sequenced. In another embodiment, a neo-epitope is identified using T-cell receptor sequencing.

In another embodiment, a neo-epitope comprises a neo-epitope known in the art, a disclosed in Pavlenko M, Leder C, Roos A K, Levitsky V, Pisa P. (2005) Identification of an immunodominant H-2D(b)-restricted CTL epitope of human PSA. Prostate. 15; 64(1):50-9 (PSA neo-epitope); Maciag P C, Seavey M M, Pan Z K, Ferrone S, Paterson Y. (2008) Cancer immunotherapy targeting the high molecular weight melanoma-associated antigen protein results in a broad antitumor response and reduction of pericytes in the tumor vasculature. Cancer Res. 1; 68(19):8066-75 (HMW-MAA epitope in HLA-A2 mice); Zhang K Q, Yang F, Ye J, Jiang M, Liu Y, Jin F S, Wu Y Z. (2012) A novel DNA/peptide combined immunotherapy induces PSCA-specific cytotoxic T-lymphocyte responses and suppresses tumor growth in experimental prostate cancer. Urology; 79(6):1410.e7-13. doi: 10.1016/j.urology.2012.02.011. Epub 2012 Apr. 17 (HLA-A2 epitope PSCA); Kouiayskaia D V, Berard C A, Datena E, Hussain A, Dawson N, Klyushnenkova E N, Alexander R B. (2009) Vaccination with agonist peptide PSA: 154-163 (155L) derived from prostate specific antigen induced CD8 T-cell response to the native peptide PSA: 154-163 but failed to induce the reactivity against tumor targets expressing PSA: a phase 2 study in patients with recurrent prostate cancer J. Immunother.; 32(6):655-66 (HLA-A2 epitope PSA).

The term “genome” relates to the total amount of genetic information in the chromosomes of an organism. The term “exome” refers to the coding regions of a genome. The term “transcriptome” relates to the set of all RNA molecules.

A nucleic acid is according to one embodiment, deoxyribonucleic acid (DNA) or ribonucleic acid (RNA), more preferably RNA, most preferably in vitro transcribed RNA (Fv RNA) or synthetic RNA. Nucleic acids include according to the invention genomic DNA, cDNA, mRNA, recombinantly produced and chemically synthesized molecules. In another embodiment, a nucleic acid may be present as a single-stranded or double-stranded and linear or covalently circularly closed molecule. A nucleic acid may, in another embodiment, be isolated. The term “isolated nucleic acid” means, according to the invention, that the nucleic acid (i) was amplified in vitro, for example via polymerase chain reaction (PCR), (ii) was produced recombinantly by cloning, (iii) was purified, for example, by cleavage and separation by gel electrophoresis, or (iv) was synthesized, for example, by chemical synthesis. A nucleic can be employed for introduction into, i.e. transfection of, cells, in particular, in the form of RNA which can be prepared by in vitro transcription from a DNA template. The RNA can moreover be modified before application by stabilizing sequences, capping, and polyadenylation.

It would be understood by a skilled artisan that the term “mutation” may encompass a change of or difference in the nucleic acid sequence (nucleotide substitution, addition or deletion, early termination or stop) compared to a reference sequence. For example a change or difference present in the abnormal sample not found in the normal sample. A “somatic mutation” can occur in any of the cells of the body except the germ cells (sperm and egg) and therefore are not passed on to children. These alterations can (but do not always) cause cancer or other diseases. In one embodiment, a mutation is a non-synonymous mutation. The term “non-synonymous mutation” refers to a mutation, preferably a nucleotide substitution, which does result in an amino acid change such as an amino acid substitution in the translation product.

In the case of an abnormal sample being a tumor or cancer tissue, in one embodiment, a mutation may comprise a “cancer mutation signature.” The term “cancer mutation signature” refers to a set of mutations which are present in cancer cells when compared to non-cancerous reference cells. Included are pre-cancerous or dysplastic tissue, and somatic mutations of same.

Digital karyotyping is a technique used to analyze chromosomes in order to look for any major chromosomal anomaly which may cause a genetic condition. In one embodiment, digital karyotyping may be used to focus on regions of a chromosome for sequencing and comparative analysis. In another embodiment, digital karyotyping is performed virtually analyzing short sequences of DNA from specific loci all over the genome, which are isolated and enumerated.

Any suitable sequencing method can be used according to the invention. In one embodiment, next Generation Sequencing (NGS) technologies is used. Third Generation Sequencing methods might substitute for the NGS technology in the future to speed up the sequencing step of the method. For clarification purposes: the terms “Next Generation Sequencing” or “NGS” in the context of the present invention mean all novel high throughput sequencing technologies which, in contrast to the “conventional” sequencing methodology known as Sanger chemistry, read nucleic acid templates randomly in parallel along the entire genome by breaking the entire genome into small pieces. Such NGS technologies (also known as massively parallel sequencing technologies) are able to deliver nucleic acid sequence information of a whole genome, exome, transcriptome (all transcribed sequences of a genome) or methylome (all methylated sequences of a genome) in very short time periods, e.g. within 1-2 weeks, preferably within 1-7 days or most preferably within less than 24 hours and allow, in principle, single cell sequencing approaches. Multiple NGS platforms which are commercially available or which are mentioned in the literature can be used in the context of the present invention e.g. those described in detail in Zhang et al. 2011: The impact of next-generation sequencing on genomics. J. Genet Genomics 38 (3), 95-109; or in Voelkerding et al. 2009: Next generation sequencing: From basic research to diagnostics. Clinical chemistry 55, 641-658. Non-limiting examples of such NGS technologies/platforms include:

1) The sequencing-by-synthesis technology known as pyrosequencing implemented e.g. in the GS-FLX 454 Genome Sequencer™ of Roche-associated company 454 Life Sciences (Branford, Conn.), first described in Ronaghi et al. 1998: A sequencing method based on real-time pyrophosphate”. Science 281 (5375), 363-365. This technology uses an emulsion PCR in which single-stranded DNA binding beads are encapsulated by vigorous vortexing into aqueous micelles containing PCR reactants surrounded by oil for emulsion PCR amplification. During the pyrosequencing process, light emitted from phosphate molecules during nucleotide incorporation is recorded as the polymerase synthesizes the DNA strand.

2) The sequencing-by-synthesis approaches developed by Solexa (now part of Illumina Inc., San Diego, Calif.) which is based on reversible dye-terminators and implemented e.g. in the Illumina Solexa Genome Analyzer™ and in the Illumina HiSeq 2000 Genome Analyzer™. In this technology, all four nucleotides are added simultaneously into oligo-primed cluster fragments in flow-cell channels along with DNA polymerase. Bridge amplification extends cluster strands with all four fluorescently labeled nucleotides for sequencing.

3) Sequencing-by-ligation approaches, e.g. implemented in the SOLid™ platform of Applied Biosystems (now Life Technologies Corporation, Carlsbad, Calif.). In this technology, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting bead, each containing only copies of the same DNA molecule, are deposited on a glass slide. As a second example, the Polonator™ G.007 platform of Dover Systems (Salem, N.H.) also employs a sequencing-by-ligation approach by using a randomly arrayed, bead-based, emulsion PCR to amplify DNA fragments for parallel sequencing.

4) Single-molecule sequencing technologies such as e.g. implemented in the PacBio RS system of Pacific Biosciences (Menlo Park, Calif.) or in the HeliScope™ platform of Helicos Biosciences (Cambridge, Mass.). The distinct characteristic of this technology is its ability to sequence single DNA or RNA molecules without amplification, defined as Single-Molecule Real Time (SMRT) DNA sequencing. For example, HeliScope uses a highly sensitive fluorescence detection system to directly detect each nucleotide as it is synthesized. A similar approach based on fluorescence resonance energy transfer (FRET) has been developed from Visigen Biotechnology (Houston, Tex.). Other fluorescence-based single-molecule techniques are from U.S. Genomics (GeneEngine™) and Genovoxx (AnyGene™)

5) Nano-technologies for single-molecule sequencing in which various nano structures are used which are, e.g., arranged on a chip to monitor the movement of a polymerase molecule on a single strand during replication. Non-limiting examples for approaches based on nano-technologies are the GridON™ platform of Oxford Nanopore Technologies (Oxford, UK), the hybridization-assisted nano-pore sequencing (HANS™) platforms developed by Nabsys (Providence, R.I.), and the proprietary ligase-based DNA sequencing platform with DNA nanoball (DNB) technology called combinatorial probe-anchor ligation (cPAL™).

6) Electron microscopy based technologies for single-molecule sequencing, e.g. those developed by LightSpeed Genomics (Sunnyvale, Calif.) and Halcyon Molecular (Redwood City, Calif.)

7) Ion semiconductor sequencing which is based on the detection of hydrogen ions that are released during the polymerisation of DNA. For example, Ion Torrent Systems (San Francisco, Calif.) uses a high-density array of micro-machined wells to perform this biochemical process in a massively parallel way. Each well holds a different DNA template. Beneath the wells is an ion-sensitive layer and beneath that a proprietary Ion sensor.

In some embodiments, DNA and RNA preparations serve as starting material for NGS. Such nucleic acids can be easily obtained from samples such as biological material, e.g. from fresh, flash-frozen or formalin-fixed paraffin embedded tumor tissues (FFPE) or from freshly isolated cells or from CTCs which are present in the peripheral blood of patients. Normal non-mutated genomic DNA or RNA can be extracted from normal, somatic tissue, however germline cells are preferred in the context of the present invention. Germline DNA or RNA is extracted from peripheral blood mononuclear cells (PBMCs) in patients with non-hematological malignancies. Although nucleic acids extracted from FFPE tissues or freshly isolated single cells are highly fragmented, they are suitable for NGS applications.

Several targeted NGS methods for exome sequencing are described in the literature (for review see e.g. Teer and Mullikin 2010: Human Mol Genet 19 (2), R145-51), all of which can be used in conjunction with the present invention. Many of these methods (described e.g. as genome capture, genome partitioning, genome enrichment etc.) use hybridization techniques and include array-based (e.g. Hodges et al. 2007: Nat. Genet. 39, 1522-1527) and liquid-based (e.g. Choi et al. 2009: Proc. Natl. Acad. Sci USA 106, 19096-19101) hybridization approaches. Commercial kits for DNA sample preparation and subsequent exome capture are also available: for example, Illumina Inc. (San Diego, Calif.) offers the TruSeq™ DNA Sample Preparation Kit and the Exome Enrichment Kit TruSeq™ Exome Enrichment Kit.

In the context of the present invention, the term “RNA” relates to a molecule which comprises at least one ribonucleotide residue and preferably being entirely or substantially composed of ribonucleotide residues. “Ribonucleotide” relates to a nucleotide with a hydroxyl group at the 2′-position of a β-D-ribofuranosyl group. The term “RNA” comprises double-stranded RNA, single-stranded RNA, isolated RNA such as partially or completely purified RNA, essentially pure RNA, synthetic RNA, and recombinantly generated RNA such as modified RNA which differs from naturally occurring RNA by addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally-occurring RNA.

According to the present invention, the term “RNA” includes and preferably relates to “mRNA”. The term “mRNA” means “messenger-RNA” and relates to a “transcript” which is generated by using a DNA template and encodes a peptide or polypeptide. Typically, an mRNA comprises a 5′-UTR, a protein coding region, and a 3′-UTR. mRNA only possesses limited half-life in cells and in vitro. In the context of the present invention, mRNA may be generated by in vitro transcription from a DNA template. The in vitro transcription methodology is known to the skilled person. For example, there is a variety of in vitro transcription kits commercially available.

In one embodiment, DNA and RNA from a biological sample (disease and/or normal) obtained from human tissue (or any non-human animal) are extracted in triplicates. In another embodiment, the disease sample is a tumor sample and said sample provides the source of neo-antigens/neo-epitopes. In another embodiment, a source of neo-antigens is from sequencing metastases or circulating tumor cells. It will be appreciated by a skilled artisan that these may contain additional mutations that are not resident in the initial biopsy but could be included in the vector to specifically target cytotoxic T cells (CTC's) or metastases that have mutated differently than the primary biopsy that was sequenced.

In one embodiment, triplicates of each sample obtained according to the disclosure herein are sequenced by DNA exome sequencing. Following a whole exome sequencing a VCF file output data or other suitable file is obtained and is presented in the FASTA format or any other suitable format known in the art. In one embodiment, the term “VCF” or Variant Call Format is a file format used by the 1000 Genomes project to encode SNPs and other structural genetic variants. The format is further described on the 1000 Genomes project Web site (www.1000genomes.org/wiki/Analysis/Variant %20Call %20Format/VCF%20%28Variant%20Call%20Format%29%20version%204.0/encoding-structural-variants). VCF calls are available at EBI/NCBI. In one embodiment, the presentation places the non-synonymous mutation in the center and shows 10-15 amino acids on either side of the mutation encoded amino acid. Frame shift mutations will display the entire sequence of the mutated peptide that is encoded until a stop codon with the surrounding 10-15 amino acids. In one embodiment, extracting the relevant information from a VCF or other suitable file and putting it in FASTA or other suitable format allows for direct input of the 21mer neo-epitope sequences into both hydropathy testing and MHC binding affinity scripts.

In one embodiment, the hydrophobicity is scaled using the Kyte-Doolittle (Kyte J, Doolittle RF (May 1982). “A simple method for displaying the hydropathic character of a protein”. J. Mol. Biol. 157 (1): 105-32) or other suitable hydropathy plot or other appropriate scale including, but not limited those disclosed by Rose et. al (Rose, G. D. and Wolfenden, R. (1993) Annu. Rev. Biomol. Struct., 22, 381-415.); Kallol M. Biswas, Daniel R. DeVido, John G. Dorsey (2003) Journal of Chromatography A, 1000, 637-655, Eisenberg D (July 1984). Ann. Rev. Biochem. 53: 595-623.); Abraham D. J., Leo A. J. Proteins: Structure, Function and Genetics 2:130-152 (1987); Sweet R. M., Eisenberg D. J. Mol. Biol. 171:479-488 (1983); Bull H. B., Breese K. Arch. Biochem. Biophys. 161:665-670 (1974); Guy H. R.

Biophys J. 47:61-70 (1985); Miyazawa S., et al., Macromolecules 18:534-552 (1985); Roseman M. A. J. Mol. Biol. 200:513-522 (1988); Wolfenden R. V., et al. Biochemistry 20:849-855 (1981); Wilson K. J; Biochem. J. 199:31-41 (1981); Cowan R., Whittaker R. G. Peptide Research 3:75-80 (1990); Aboderin A. A. Int. J. Biochem. 2:537-544 (1971); Eisenberg D. et al., J. Mol. Biol. 179:125-142 (1984); Hopp T. P., Woods K. R. Proc. Natl. Acad. Sci. U.S.A. 78:3824-3828 (1981); Manavalan P., Ponnuswamy P. K. Nature 275:673-674 (1978).; Black S. D., Mould D. R. Anal. Biochem. 193:72-82 (1991); Fauchere J.-L., Pliska V. E. Eur. J. Med. Chem. 18:369-375 (1983); Janin J. Nature 277:491-492 (1979); Rao M. J. K., Argos P. Biochim Biophys. Acta 869:197-214 (1986); Tanford C. J. Am. Chem. Soc. 84:4240-4274 (1962); Welling G. W., et al., FEBS Lett. 188:215-218 (1985); Parker J. M. R. et al., Biochemistry 25:5425-5431 (1986); Cowan R., Whittaker R. G. Peptide Research 3:75-80 (1990), all of which are incorporated by reference herein in their entirety. In another embodiment, all epitopes scoring on the scale-appropriate measure to have an unsatisfactorily high level of hydrophobicity to be efficiently secreted are moved from the listing or are de-selected. In another embodiment, all epitopes scoring on the Kyte-Doolittle plot to have an unsatisfactorily high level of hydrophobicity to be efficiently secreted, such as 1.6 or above, are moved from the listing or are de-selected. In another embodiment, each neo-antigen's ability to bind to subject HLA is rated using the Immune Epitope Database (IEDB) analysis resource which includes: netMHCpan, ANN, SMMPMBEC. SMM, CombLib_Sidney2008, PickPocket, netMHCcons. Other sources include TEpredict (tepredict.sourceforge.net/help.html) or alternative MHC binding measurement scales available in the art.

In another embodiment, disclosed herein is a system for creating personalized immunotherapy for a subject, comprising: at least one processor; and at least one storage medium containing program instructions for execution by said processor, said program instructions causing said processor to execute steps comprising:
a. Receiving output data containing all neo-antigens/neo-epitopes and the human leukocyte antigen (HLA) type of the subject;
b. Scoring the hydrophobicity of each epitope and removing epitopes that score above a certain threshold;
c. Numerically rate the remaining neo-antigens based its ability to bind to subject HLA and on its predictive MHC binding score;
d. Inserting the amino acid sequence of each epitope into a plasmid;
e. Scoring the hydrophobicity of each construct and removing any constructs that score above a certain threshold;
f. Translating the amino acid sequence of each construct into the corresponding DNA sequence, starting with the highest scored construct;
g. Inserting additional epitopes into the plasmid construct in order of ranking until a predetermined upper limit is reached;
h. Adding a DNA sequence tag to the end of the construct in order to measure the immunotherapeutic response in a subject; and
i. Optimizing the epitope and DNA sequence tag for expression and secretion in Listeria monocytogenes.

In one embodiment, once a neo-epitope is identified, the neo-epitope is scored by the Kyte and Doolittle hydropathy index 21 amino acid window, wherein in another embodiment, neo-epitopes scoring above a specific cutoff (around 1.6) are excluded as they are unlikely to be secretable by Listeria monocytogenes. In another embodiment, the cut off is selected from the following ranges 1.2-1.4, 1.4-1.6, 1.6-1.8, 1.8-2.0, 2.0-2.2 2.2-2.5, 2.5-3.0, 3.0-3.5, 3.5-4.0, or 4.0-4.5. In one embodiment, embodiment the cutoff score used to determine what epitopes are moved from the list or are de-selected is 1.6. In another embodiment, the cutoff is 1.4, 1.5, 1.7, 1.8, 1.9, 2.0, 2.1, 2.2, 2.3, 2.3, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5. In another embodiment, the cut off varies depending on the genus of the delivery vector being used. In another embodiment, the cut off varies depending on the species of the delivery vector being used.

In one embodiment, the neo-epitope is scored by the Kyte and Doolittle hydropathy index 21 amino acid sliding window. In another embodiment, the sliding window size is selected from the group comprising 9, 11, 13, 15, 17, 19, and 21 amino acids. In another embodiment, the sliding window size is 9-11 amino acids, 11-13 amino acids, 13-15 amino acids, 15-17 amino acids, 17-19 amino acids or 19-21 amino acids.

In another embodiment, wherein the DNA sequence tag of step h. is SIINFEKL-6×His or a substitute tag sequence available in the art. In another embodiment, neo-antigens known to have immunosuppressive properties are removed from consideration before step a. above. In one embodiment, these immunosuppressive epitopes are as presented in the sequence or are artificially created as a result of the splicing together of epitope sequences and linkers.

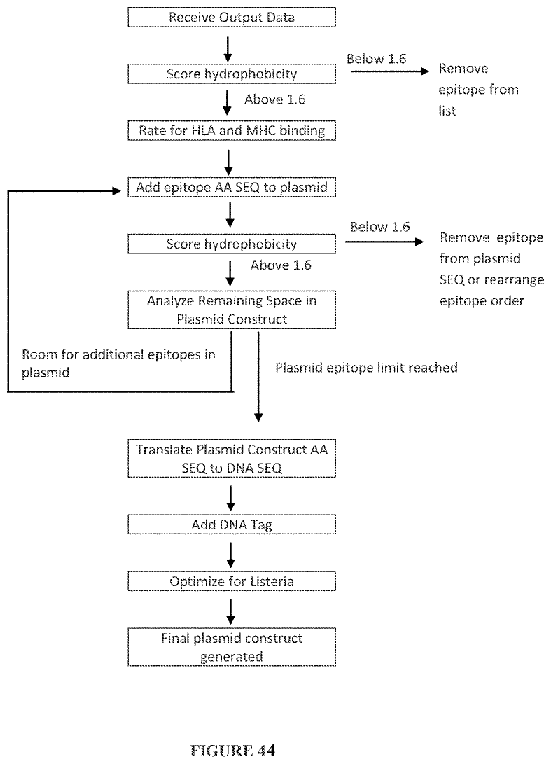

In one embodiment, an output FASTA file obtained by the process disclosed herein (see e.g., Example 30 herein) is used to design patient-specific constructs, either manually or by programmed script. In another embodiment, the programmed script automates the creation of the personalized plasma construct containing one or more neo-epitopes for each subject using a series of protocols (FIG. 44). The output FASTA file is inputted and after running the protocols, the DNA sequence of a LM vector including one or more neo-epitopes is outputted. The software program is useful for creating personalized immunotherapy for each subject.

In one embodiment, the nucleic acid sequences from disease-bearing and healthy samples are compared in order to identify neo-epitopes. Neo-epitopes comprise amino acid sequences changes within ORF sequences. As used herein, the term “sequence change” with respect to peptides or proteins relates to amino acid insertion variants, amino acid addition variants, amino acid deletion variants and amino acid substitution variants, preferably amino acid substitution variants. All these sequence changes according to the invention may potentially create new epitopes.

In one embodiment, amino acid insertion variants comprise insertions of single or two or more amino acids in a particular amino acid sequence. In another embodiment, amino acid addition variants comprise amino- and/or carboxy-terminal fusions of one or more amino acids, such as 1, 2, 3, 4 or 5, or more amino acids. In another embodiment, amino acid deletion variants are characterized by the removal of one or more amino acids from the sequence, such as by removal of 1, 2, 3, 4 or 5, or more amino acids. In another embodiment, amino acid substitution variants are characterized by at least one residue in the sequence being removed and another residue being inserted in its place.

All samples are analyzed for novel genetic sequencing within ORFs. Methods for comparing one or more open reading frames (ORFs) in nucleic acid sequences extracted from said disease-bearing biological sample and healthy biological sample comprise the use of screening assays or screening tools and associated digital software. Methods for performing bioinformatics analyses are known in the art, for example, see US Publication Nos. US 2013/0210645, US 2014/0045881, and International Publication WO 2014/052707, which are each incorporated in full in this application.

Human tumors typically harbor a remarkable number of somatic mutations. Yet, identical mutations in any particular gene are rarely found across tumors (and are even at low frequency for the most common driver mutations). Thus, in one embodiment, a process of this invention comprehensively identifying patient-specific tumor mutations provides a target for a personalized immunotherapy.

In one embodiment, mutations identifying from a disease-bearing sample may be presented on major histocompatibility complex class I molecules (MHCI). In one embodiment, a peptides containing a neo-epitope mutation is immunogenic and is recognized as a ‘non-self’ neo-antigens by the adaptive immune system. In another embodiment, use of one or more neo-epitope sequences comprised in a peptide, a polypeptide, or a fusion polypeptide provides a targeting immunotherapy, which may, in certain embodiments therapeutically activate a T-cell immune responses to said disease or condition. In another embodiment, use of one or more neo-epitope sequences comprised in a peptide, a polypeptide, or a fusion polypeptide provides a targeting immunotherapy, which may, in certain embodiments therapeutically activate an adaptive immune responses to a disease or condition.

In another embodiment, one or more neo-epitope sequences comprised in a peptide, a polypeptide, or a fusion polypeptide is used to provide a therapeutic anti-tumor or anti-cancer T-cell immune response. In another embodiment, use of one or more neo-epitope sequences comprised in a peptide, a polypeptide, or a fusion polypeptide provides a targeting immunotherapy, which may, in certain embodiments therapeutically activate an anti-tumor or anti-cancer adaptive immune response. In another embodiment, one or more neo-epitope sequences comprised in a peptide, a polypeptide, or a fusion polypeptide is used to provide a therapeutic anti-autoimmune disease T-cell immune response. In another embodiment, use of one or more neo-epitope sequences comprised in a peptide, a polypeptide, or a fusion polypeptide provides a targeting immunotherapy, which may, in certain embodiments therapeutically activate an anti-autoimmune disease adaptive immune response. In another embodiment, a one or more neo-epitope sequence comprised in a peptide, a polypeptide, or a fusion polypeptide is use to provide a therapeutic anti-infectious disease T-cell immune response. In another embodiment, use of one or more neo-epitope sequences comprised in a peptide, a polypeptide, or a fusion polypeptide provides a targeting immunotherapy, which may, in certain embodiments therapeutically activate an anti-infectious disease adaptive immune response. In another embodiment, one or more neo-epitope sequences comprised in a peptide, a polypeptide, or a fusion polypeptide is used to provide a therapeutic anti-organ transplantation rejection T-cell immune response. In another embodiment, use of a one or more neo-epitope sequence comprised in a peptide, a polypeptide, or a fusion polypeptide provides a targeting immunotherapy, which may, in certain embodiments therapeutically activate an anti-organ transplantation rejection adaptive immune response.

In another embodiment, wherein the presence of an immunogenic response correlates with a presence of one or more immunogenic neo-epitopes. In another embodiment, a recombinant Listeria comprises nucleic acid encoding neo-epitopes comprising T-cell epitopes, or adaptive immune response epitopes, or any combination thereof.

In one embodiment, the process comprises screening each amino acid sequence comprising one or more neo-epitopes for an immunogenic response, wherein the presence of an immunogenic response correlates with one or more neo-epitopes comprising an immunogenic epitope. In another embodiment, one or more immunogenic neo-epitopes is comprised in a peptide. In another embodiment, one or more immunogenic neo-epitopes is comprised in a polypeptide. In another embodiment, one or more immunogenic neo-epitopes is comprised in a fusion-polypeptide. In another embodiment, one or more immunogenic neo-epitopes is comprised fused to a ubiquitin polypeptide.

In another embodiment, the process comprises screening each amino acid sequence comprising one or more neo-epitopes for an immunogenic T-cell response, wherein the presence of an immunogenic T-cell response correlates with one or more neo-epitopes comprising a T-cell epitope. In another embodiment, the process comprises screening each amino acid sequence comprising one or more neo-epitopes for an adaptive immune response, wherein the presence of an adaptive immune response correlates with one or more neo-epitopes comprising an adaptive immune response epitope.

In one embodiment, a step of screening for an immunogenic T-cell response in the system or process of creating a personalized immunotherapy provided comprises use of an immune response assay well known in the art, including for example T-cell proliferation assays, in vitro tumor regression assays using T-cells activated with said neo-epitope and co-incubated with tumor cells using a ⁵¹Cr-release assay or a ³H-thymidine assay, an ELISA assay, an ELIspot assay, and a FACS analysis. (See for example U.S. Pat. No. 8,771,702, and European Patent No. EP_1774332_B1, which are incorporated herein in their entirety). In another embodiment, a step for screening for an immunogenic response examines a non-T-cell response. In another embodiment, a step of screening for a non-T-cell response in the system or process of creating a personalized immunotherapy provided comprises use of an immune response assay well known in the art, including for example an assay similar to those above for T-cells, except that examining cytokine production focuses on a different subset of cytokines, namely, IL-10 and IL-1β. (See for example U.S. Pat. No. 8,962,319 and EP 177432, both of which are incorporated in full herein. For example, a T-cell immune response may be assayed by a ⁵¹Cr release assay, comprising the steps of immunizing mice with a immunotherapy comprising one or more neo-epitopes, followed by harvesting spleens about ten days post-immunization, wherein splenocytes may then be established in culture with irradiated TC-1 cells (100:1, splenocytes:TC-1) as feeder cells; stimulated in vitro for 5 days, then used in a standard ⁵¹Cr release assay, using a peptide/polypeptide comprising one or more neo-epitopes as the target.

In another embodiment, a step for screening for an immune response comprises use of an HLA-A2 transgenic mouse, for example as disclosed in US Patent Application Publication No.: US-2011-0129499, which is incorporated in full herein.

In one embodiment, the process comprises selecting a nucleic acid sequence that encodes an identified T-cell neo epitope or encodes a peptide comprising said identified T-cell neo-epitope, and transforming said sequence into a recombinant attenuated Listeria strain. In one embodiment, the process comprises selecting a nucleic acid sequence that encodes an identified adaptive immune response neo-epitope or encodes a peptide comprising said identified adaptive immune response neo-epitope, and transforming said sequence into a recombinant attenuated Listeria strain.

In one embodiment, the system or process described herein comprises culturing and characterizing said Listeria strain to confirm expression and secretion of said T-cell neo-epitope. In one embodiment, the system or process described herein comprises culturing and characterizing said Listeria strain to confirm expression and secretion of said adaptive immune response neo-epitope. In one embodiment, the system or process described herein comprises culturing and characterizing said Listeria strain to confirm expression and secretion of said one or more peptides.

In one embodiment, the system or process of this invention comprises storing said Listeria for administrating to said subject at a pre-determined period or administering said Listeria to said subject, wherein said Listeria strain is administered as part of an immunogenic composition.

### II. Recombinant Listeria Strains

In one embodiment, a recombinant Listeria strain of the present invention comprises a nucleic acid molecule, the nucleic acid molecule comprising a first open reading frame encoding a fusion polypeptide, wherein the fusion polypeptide comprises a truncated listeriolysin O (tLLO) protein, a truncated ActA protein, or a PEST amino acid sequence fused to one or more peptides comprising one or more neo-epitopes. It will be understood by a skilled artisan that one or more peptides provided herein which comprise one or more epitopes may be immunogenic to start with and their immunogenicity may be enhanced by fusing with or mixing with an immunogenic polypeptide such as a tLLO, a truncated ActA protein or a PEST amino acid sequence. In another embodiment, a recombinant Listeria strain of the present invention comprises a nucleic acid molecule, the nucleic acid molecule comprising a first open reading frame encoding a truncated listeriolysin O (LLO) protein, a truncated ActA protein, or a PEST amino acid sequence. In one embodiment, the recombinant Listeria strain is attenuated.

In one embodiment, one or more peptides comprising one or more immunogenic neo-epitopes provided herein are each fused to an immunogenic polypeptide or fragment thereof.

In another embodiment, a truncated listeriolysin O (LLO) protein, a truncated ActA protein, or a PEST amino acid sequence is not fused to a heterologous antigen or a fragment thereof. In another embodiment, a truncated listeriolysin O (LLO) protein, a truncated ActA protein, or a PEST amino acid sequence is not fused to one or more peptides provided herein.

In another embodiment, one or more peptides comprising one or more immunogenic neo-epitopes provided herein are mixed with an immunogenic polypeptide or fragment thereof as part of an immunogenic composition.

In one embodiment, a truncated listeriolysin O (LLO) protein comprises a putative PEST sequence. In one embodiment, a truncated actA protein comprises a PEST-containing amino acid sequence. In another embodiment, a truncated actA protein comprises a putative PEST-containing amino acid sequence.

In one embodiment, a PEST amino acid (AA) sequence comprises a truncated LLO sequence. In another embodiment, the PEST amino acid sequence is KENSISSMAPPASPPASPKTPIEKKHADEIDK (SEQ ID NO: 1). In another embodiment, fusion of an antigen to other LM PEST AA sequences from Listeria will also enhance immunogenicity of the antigen.

The N-terminal LLO protein fragment of methods and compositions of the present invention comprises, in another embodiment, SEQ ID No: 3. In another embodiment, the fragment comprises an LLO signal peptide. In another embodiment, the fragment comprises SEQ ID No: 4. In another embodiment, the fragment consists approximately of SEQ ID No: 4. In another embodiment, the fragment consists essentially of SEQ ID No: 4. In another embodiment, the fragment corresponds to SEQ ID No: 4. In another embodiment, the fragment is homologous to SEQ ID No: 4. In another embodiment, the fragment is homologous to a fragment of SEQ ID No: 4. In one embodiment, a truncated LLO used excludes of the signal sequence. In another embodiment, the truncated LLO comprises a signal sequence. It will be clear to those skilled in the art that any truncated LLO without the activation domain, and in particular without cysteine 484, are suitable for methods and compositions of the present invention. In another embodiment, fusion of a heterologous antigen to any truncated LLO, including the PEST AA sequence, SEQ ID NO: 1, enhances cell mediated and anti-tumor immunity of the antigen.

The LLO protein utilized to construct immunotherapies of the present invention has, in another embodiment, the sequence: MKKIMLVFITLILVSLPIAQQTEAKDASAFNKENSISSMAPPASPPASPKTPIEKKHADE IDKYIQGLDYNKNNVLVYHGDAVTNVPPRKGYKDGNEYIVVEKKKKSINQNNADIQ VVNAISSLTYPGALVKANSELVENQPDVLPVKRDSLTLSIDLPGMTNQDNKIVVKNA TKSNVNNAVNTLVERWNEKYAQAYPNVSAKIDYDDEMAYSESQLIAKFGTAFKAV NNSLNVNFGAISEGKMQEEVISFKQIYYNVNVNEPTRPSRFFGKAVTKEQLQALGVN AENPPAYISSVAYGRQVYLKLSTNSHSTKVKAAFDAAVSGKSVSGDVELTNIIKNSSF KAVIYGGSAKDEVQIIDGNLGDLRDILKKGATFNRETPGVPIAYTTNFLKDNELAVIK NNSEYIETTSKAYTDGKINIDHSGGYVAQFNISWDEVNYDPEGNEIVQHKNWSENNK SKLAHFTSSIYLPGNARNINVYAKECTGLAWEWWRTVIDDRNLPLVKNRNISIWGTT LYPKYSNKVDNPIE (GenBank Accession No. P13128; SEQ ID NO: 2; nucleic acid sequence is set forth in GenBank Accession No. X15127). The first 25 AA of the proprotein corresponding to this sequence are the signal sequence and are cleaved from LLO when it is secreted by the bacterium. Thus, in this embodiment, the full length active LLO protein is 504 residues long. In another embodiment, the above LLO fragment is used as the source of the LLO fragment incorporated in a immunotherapy of the present invention.

In another embodiment, the N-terminal fragment of an LLO protein utilized in compositions and methods of the present invention has the sequence:

(SEQ ID NO: 3)
MKKIMLVFITLILVSLPIAQQTEAKDASAFNKENSISSVAPPASPPASPK
TPIEKKHADEIDKYIQGLDYNKNNVLVYHGDAVTNVPPRKGYKDGNEYIV
VEKKKKSINQNNADIQVVNAISSLTYPGALVKANSELVENQPDVLPVKRD
SLTLSIDLPGMTNQDNKIVVKNATKSNVNNAVNTLVERWNEKYAQAYSNV
SAKIDYDDEMAYSESQLIAKFGTAFKAVNNSLNVNFGAISEGKMQEEVIS
FKQIYYNVNVNEPTRPSRFFGKAVTKEQLQALGVNAENPPAYISSVAYGR
QVYLKLSTNSHSTKVKAAFDAAVSGKSVSGDVELTNIIKNSSFKAVIYGG
SAKDEVQIIDGNLGDLRDILKKGATFNRETPGVPIAYTTNFLKDNELAVI
KNNSEYIETTSKAYTDGKINIDHSGGYVAQFNISWDEVNYD.

In another embodiment, the LLO fragment corresponds to about AA 20-442 of an LLO protein utilized herein.

In another embodiment, the LLO fragment has the sequence:

(SEQ ID NO: 4)
MKKIMLVFITLILVSLPIAQQTEAKDASAFNKENSISSVAPPASPPASPK
TPIEKKHADEIDKYIQGLDYNKNNVLVYHGDAVTNVPPRKGYKDGNEYIV
VEKKKKSINQNNADIQVVNAISSLTYPGALVKANSELVENQPDVLPVKRD
SLTLSIDLPGMTNQDNKIVVKNATKSNVNNAVNTLVERWNEKYAQAYSNV
SAKIDYDDEMAYSESQLIAKFGTAFKAVNNSLNVNFGAISEGKMQEEVIS
FKQIYYNVNVNEPTRPSRFFGKAVTKEQLQALGVNAENPPAYISSVAYGR
QVYLKLSTNSHSTKVKAAFDAAVSGKSVSGDVELTNIIKNSSFKAVIYGG
SAKDEVQIIDGNLGDLRDILKKGATFNRETPGVPIAYTTNFLKDNELAVI
KNNSEYIETTSKAYTD.

In another embodiment, the terms “N-terminal truncated LLO protein,” “N-terminal LLO fragment,” “truncated LLO protein,” “ALLO” or their grammatical equivalents are used interchangeably herein and refers to a fragment of LLO that is non-hemolytic. In another embodiment, the terms refer to an LLO fragment that comprises a putative PEST sequence.

In another embodiment, the LLO fragment is rendered non-hemolytic by deletion or mutation of the activation domain. In another embodiment, the LLO fragment is rendered non-hemolytic by deletion or mutation of region comprising cysteine 484. In another embodiment, the LLO is rendered non-hemolytic by a deletion or mutation of the cholesterol binding domain (CBD) as detailed in U.S. Pat. No. 8,771,702, which is incorporated by reference herein.

In one embodiment, the present invention provides a recombinant protein or polypeptide comprising a listeriolysin O (LLO) protein, wherein said LLO protein comprises a mutation of residues C484, W491, W492, or a combination thereof of the cholesterol-binding domain (CBD) of said LLO protein. In one embodiment, said C484, W491, and W492 residues are residues C484, W491, and W492 of SEQ ID NO: 2, while in another embodiment, they are corresponding residues as can be deduced using sequence alignments, as is known to one of skill in the art. In one embodiment, residues C484, W491, and W492 are mutated. In one embodiment, a mutation is a substitution, in another embodiment, a deletion. In one embodiment, the entire CBD is mutated, while in another embodiment, portions of the CBD are mutated, while in another embodiment, only specific residues within the CBD are mutated.

In one embodiment, the present invention provides a recombinant protein or polypeptide comprising a mutated LLO protein or fragment thereof, wherein the mutated LLO protein or fragment thereof contains a substitution of a non-LLO peptide for a mutated region of the mutated LLO protein or fragment thereof, the mutated region comprising a residue selected from C484, W491, and W492. In another embodiment, the LLO fragment is an N-terminal LLO fragment. In another embodiment, the LLO fragment is at least 492 amino acids (AA) long. In another embodiment, the LLO fragment is 492-528 AA long. In another embodiment, the non-LLO peptide is 1-50 amino acids long. In another embodiment, the mutated region is 1-50 amino acids long. In another embodiment, the non-LLO peptide is the same length as the mutated region. In another embodiment, the non-LLO peptide has a length different from the mutated region. In another embodiment, the substitution is an inactivating mutation with respect to hemolytic activity. In another embodiment, the recombinant protein or polypeptide exhibits a reduction in hemolytic activity relative to wild-type LLO. In another embodiment, the recombinant protein or polypeptide is non-hemolytic.

As provided herein, a mutant LLO protein was created wherein residues C484,

W491, and W492 of LLO were substituted with alanine residues (Example 25). The mutated LLO protein, mutLLO, could be expressed and purified in an E. coli expression system (Example 27) and exhibited substantially reduced hemolytic activity relative to wild-type LLO (Example 28).

In another embodiment, the present invention provides a recombinant protein or polypeptide comprising (a) a mutated LLO protein, wherein the mutated LLO protein contains an internal deletion, the internal deletion comprising the cholesterol-binding domain of the mutated LLO protein; and (b) a heterologous peptide of interest. In another embodiment, the sequence of the cholesterol-binding domain is set forth in SEQ ID NO: 101. In another embodiment, the internal deletion is an 11-50 amino acid internal deletion. In another embodiment, the internal deletion is inactivating with regard to the hemolytic activity of the recombinant protein or polypeptide. In another embodiment, the recombinant protein or polypeptide exhibits a reduction in hemolytic activity relative to wild-type LLO.

In another embodiment, the present invention provides a recombinant protein or polypeptide comprising (a) a mutated LLO protein, wherein the mutated LLO protein contains an internal deletion, the internal deletion comprising a fragment of the cholesterol-binding domain of the mutated LLO protein; and (b) a heterologous peptide of interest. In another embodiment, the internal deletion is a 1-11 amino acid internal deletion. In another embodiment, the sequence of the cholesterol-binding domain is set forth in SEQ ID NO: 101. In another embodiment, the internal deletion is inactivating with regard to the hemolytic activity of the recombinant protein or polypeptide. In another embodiment, the recombinant protein or polypeptide exhibits a reduction in hemolytic activity relative to wild-type LLO.

The mutated region of methods and compositions of the present invention comprises, in another embodiment, residue C484 of SEQ ID NO: 2. In another embodiment, the mutated region comprises a corresponding cysteine residue of a homologous LLO protein. In another embodiment, the mutated region comprises residue W491 of SEQ ID NO: 2. In another embodiment, the mutated region comprises a corresponding tryptophan residue of a homologous LLO protein. In another embodiment, the mutated region comprises residue

W492 of SEQ ID NO: 2. In another embodiment, the mutated region comprises a corresponding tryptophan residue of a homologous LLO protein. Methods for identifying corresponding residues of a homologous protein are well known in the art, and include, for example, sequence alignment.

In another embodiment, the mutated region comprises residues C484 and W491. In another embodiment, the mutated region comprises residues C484 and W492. In another embodiment, the mutated region comprises residues W491 and W492. In another embodiment, the mutated region comprises residues C484, W491, and W492.

In another embodiment, the mutated region of methods and compositions of the present invention comprises the cholesterol-binding domain of the mutated LLO protein or fragment thereof. For example, a mutated region consisting of residues 470-500, 470-510, or 480-500 of SEQ ID NO: 2 comprises the CBD thereof (residues 483-493). In another embodiment, the mutated region is a fragment of the CBD of the mutated LLO protein or fragment thereof. For example, as provided herein, residues C484, W491, and W492, each of which is a fragment of the CBD, were mutated to alanine residues (Example 25). Further, as provided herein, a fragment of the CBD, residues 484-492, was replaced with a heterologous sequence from NY-ESO-1 (Example 26). In another embodiment, the mutated region overlaps the CBD of the mutated LLO protein or fragment thereof. For example, a mutated region consisting of residues 470-490, 480-488, 490-500, or 486-510 of SEQ ID NO: 2 comprises the CBD thereof. In another embodiment, a single peptide may have a deletion in the signal sequence and a mutation or substitution in the CBD. Each possibility represents a separate embodiment of the present invention.

The length of the mutated region is, in another embodiment, 1-50 AA. In another embodiment, the length is 1-11 AA. In another embodiment, the length is 2-11 AA. In another embodiment, the length is 3-11 AA. In another embodiment, the length is 4-11 AA. In another embodiment, the length is 5-11 AA. In another embodiment, the length is 6-11 AA. In another embodiment, the length is 7-11 AA. In another embodiment, the length is 8-11 AA. In another embodiment, the length is 9-11 AA. In another embodiment, the length is 10-11 AA. In another embodiment, the length is 1-2 AA. In another embodiment, the length is 1-3 AA. In another embodiment, the length is 1-4 AA. In another embodiment, the length is 1-5 AA. In another embodiment, the length is 1-6 AA. In another embodiment, the length is 1-7 AA. In another embodiment, the length is 1-8 AA. In another embodiment, the length is 1-9 AA. In another embodiment, the length is 1-10 AA. In another embodiment, the length is 2-3 AA. In another embodiment, the length is 2-4 AA. In another embodiment, the length is 2-5 AA. In another embodiment, the length is 2-6 AA. In another embodiment, the length is 2-7 AA. In another embodiment, the length is 2-8 AA. In another embodiment, the length is 2-9 AA. In another embodiment, the length is 2-10 AA. In another embodiment, the length is 3-4 AA. In another embodiment, the length is 3-5 AA. In another embodiment, the length is 3-6 AA. In another embodiment, the length is 3-7 AA. In another embodiment, the length is 3-8 AA. In another embodiment, the length is 3-9 AA. In another embodiment, the length is 3-10 AA. In another embodiment, the length is 11-50 AA. In another embodiment, the length is 12-50 AA. In another embodiment, the length is 11-15 AA. In another embodiment, the length is 11-20 AA. In another embodiment, the length is 11-25 AA. In another embodiment, the length is 11-30 AA. In another embodiment, the length is 11-35 AA. In another embodiment, the length is 11-40 AA. In another embodiment, the length is 11-60 AA. In another embodiment, the length is 11-70 AA. In another embodiment, the length is 11-80 AA. In another embodiment, the length is 11-90 AA. In another embodiment, the length is 11-100 AA. In another embodiment, the length is 11-150 AA. In another embodiment, the length is 15-20 AA. In another embodiment, the length is 15-25 AA. In another embodiment, the length is 15-30 AA. In another embodiment, the length is 15-35 AA. In another embodiment, the length is 15-40 AA. In another embodiment, the length is 15-60 AA. In another embodiment, the length is 15-70 AA. In another embodiment, the length is 15-80 AA. In another embodiment, the length is 15-90 AA. In another embodiment, the length is 15-100 AA. In another embodiment, the length is 15-150 AA. In another embodiment, the length is 20-25 AA. In another embodiment, the length is 20-30 AA. In another embodiment, the length is 20-35 AA. In another embodiment, the length is 20-40 AA. In another embodiment, the length is 20-60 AA. In another embodiment, the length is 20-70 AA. In another embodiment, the length is 20-80 AA. In another embodiment, the length is 20-90 AA. In another embodiment, the length is 20-100 AA. In another embodiment, the length is 20-150 AA. In another embodiment, the length is 30-35 AA. In another embodiment, the length is 30-40 AA. In another embodiment, the length is 30-60 AA. In another embodiment, the length is 30-70 AA. In another embodiment, the length is 30-80 AA. In another embodiment, the length is 30-90 AA. In another embodiment, the length is 30-100 AA. In another embodiment, the length is 30-150 AA. Each possibility represents another embodiment of the present invention.

The substitution mutation of methods and compositions of the present invention is, in another embodiment, a mutation wherein the mutated region of the LLO protein or fragment thereof is replaced by an equal number of heterologous AA. In another embodiment, a larger number of heterologous AA than the size of the mutated region is introduced. In another embodiment, a smaller number of heterologous AA than the size of the mutated region is introduced. Each possibility represents another embodiment of the present invention.

In another embodiment, the substitution mutation is a point mutation of a single residue. In another embodiment, the substitution mutation is a point mutation of 2 residues. In another embodiment, the substitution mutation is a point mutation of 3 residues. In another embodiment, the substitution mutation is a point mutation of more than 3 residues. In another embodiment, the substitution mutation is a point mutation of several residues. In another embodiment, the multiple residues included in the point mutation are contiguous. In another embodiment, the multiple residues are not contiguous.

The length of the non-LLO peptide that replaces the mutated region of recombinant protein or polypeptides of the present invention is, in another embodiment, 1-50 AA. In another embodiment, the length is 1-11 AA. In another embodiment, the length is 2-11 AA. In another embodiment, the length is 3-11 AA. In another embodiment, the length is 4-11 AA. In another embodiment, the length is 5-11 AA. In another embodiment, the length is 6-11 AA. In another embodiment, the length is 7-11 AA. In another embodiment, the length is 8-11 AA. In another embodiment, the length is 9-11 AA. In another embodiment, the length is 10-11 AA. In another embodiment, the length is 1-2 AA. In another embodiment, the length is 1-3 AA. In another embodiment, the length is 1-4 AA. In another embodiment, the length is 1-5 AA. In another embodiment, the length is 1-6 AA. In another embodiment, the length is 1-7 AA. In another embodiment, the length is 1-8 AA. In another embodiment, the length is 1-9 AA. In another embodiment, the length is 1-10 AA. In another embodiment, the length is 2-3 AA. In another embodiment, the length is 2-4 AA. In another embodiment, the length is 2-5 AA. In another embodiment, the length is 2-6 AA. In another embodiment, the length is 2-7 AA. In another embodiment, the length is 2-8 AA. In another embodiment, the length is 2-9 AA. In another embodiment, the length is 2-10 AA. In another embodiment, the length is 3-4 AA. In another embodiment, the length is 3-5 AA. In another embodiment, the length is 3-6 AA. In another embodiment, the length is 3-7 AA. In another embodiment, the length is 3-8 AA. In another embodiment, the length is 3-9 AA. In another embodiment, the length is 3-10 AA. In another embodiment, the length is 11-50 AA. In another embodiment, the length is 12-50 AA. In another embodiment, the length is 11-15 AA. In another embodiment, the length is 11-20 AA. In another embodiment, the length is 11-25 AA. In another embodiment, the length is 11-30 AA. In another embodiment, the length is 11-35 AA. In another embodiment, the length is 11-40 AA. In another embodiment, the length is 11-60 AA. In another embodiment, the length is 11-70 AA. In another embodiment, the length is 11-80 AA. In another embodiment, the length is 11-90 AA. In another embodiment, the length is 11-100 AA. In another embodiment, the length is 11-150 AA. In another embodiment, the length is 15-20 AA. In another embodiment, the length is 15-25 AA. In another embodiment, the length is 15-30 AA. In another embodiment, the length is 15-35 AA. In another embodiment, the length is 15-40 AA. In another embodiment, the length is 15-60 AA. In another embodiment, the length is 15-70 AA. In another embodiment, the length is 15-80 AA. In another embodiment, the length is 15-90 AA. In another embodiment, the length is 15-100 AA. In another embodiment, the length is 15-150 AA. In another embodiment, the length is 20-25 AA. In another embodiment, the length is 20-30 AA. In another embodiment, the length is 20-35 AA. In another embodiment, the length is 20-40 AA. In another embodiment, the length is 20-60 AA. In another embodiment, the length is 20-70 AA. In another embodiment, the length is 20-80 AA. In another embodiment, the length is 20-90 AA. In another embodiment, the length is 20-100 AA. In another embodiment, the length is 20-150 AA. In another embodiment, the length is 30-35 AA. In another embodiment, the length is 30-40 AA. In another embodiment, the length is 30-60 AA. In another embodiment, the length is 30-70 AA. In another embodiment, the length is 30-80 AA. In another embodiment, the length is 30-90 AA. In another embodiment, the length is 30-100 AA. In another embodiment, the length is 30-150 AA.

In another embodiment, the length of the LLO fragment of methods and compositions of the present invention is at least 484 AA. In another embodiment, the length is over 484 AA. In another embodiment, the length is at least 489 AA. In another embodiment, the length is over 489. In another embodiment, the length is at least 493 AA. In another embodiment, the length is over 493. In another embodiment, the length is at least 500 AA. In another embodiment, the length is over 500. In another embodiment, the length is at least 505 AA. In another embodiment, the length is over 505. In another embodiment, the length is at least 510 AA. In another embodiment, the length is over 510. In another embodiment, the length is at least 515 AA. In another embodiment, the length is over 515. In another embodiment, the length is at least 520 AA. In another embodiment, the length is over 520. In another embodiment, the length is at least 525 AA. In another embodiment, the length is over 520. When referring to the length of an LLO fragment herein, the signal sequence is included. Thus, the numbering of the first cysteine in the CBD is 484, and the total number of AA residues is 529.

In another embodiment, the present invention provides a recombinant protein or polypeptide, or an attenuated Listeria strain provided herein comprising the same, comprising (a) a mutated LLO protein, wherein the mutated LLO protein contains an internal deletion, the internal deletion comprising the cholesterol-binding domain of the mutated LLO protein; and (b) peptide comprising one or more epitopes provided herein. In another embodiment, the sequence of the cholesterol-binding domain is set forth in SEQ ID NO: 101. In another embodiment, the internal deletion is a 1-11, 1-50 or an 11-50 amino acid internal deletion. In another embodiment, the internal deletion is inactivating with regard to the hemolytic activity of the recombinant protein or polypeptide. In another embodiment, the recombinant protein or polypeptide exhibits a reduction in hemolytic activity relative to wild-type LLO.

In another embodiment, a peptide of the present invention is a fusion peptide. In another embodiment, “fusion peptide” refers to a peptide or polypeptide comprising two or more proteins linked together by peptide bonds or other chemical bonds. In another embodiment, the proteins are linked together directly by a peptide or other chemical bond. In another embodiment, the proteins are linked together with one or more AA (e.g. a “spacer”) between the two or more proteins.

As provided herein, a mutant LLO protein was created wherein residues C484, W491, and W492 of LLO were substituted with a CTL epitope from the antigen NY-ESO-1 (Example 26). The mutated LLO protein, mutLLO, could be expressed and purified in an E. coli expression system (Example 2 7) and exhibited substantially reduced hemolytic activity relative to wild-type LLO (Example 28). It will be appreciated by a skilled artisan that any neo-epitope identified by the methods or processes provided herein can be used for substituting or replacing the CBD of LLO.

The length of the internal deletion of methods and compositions of the present invention is, in another embodiment, 1-50 AA. In another embodiment, the length is 1-11 AA. In another embodiment, the length is 2-11 AA. In another embodiment, the length is 3-11 AA. In another embodiment, the length is 4-11 AA. In another embodiment, the length is 5-11 AA. In another embodiment, the length is 6-11 AA. In another embodiment, the length is 7-11 AA. In another embodiment, the length is 8-11 AA. In another embodiment, the length is 9-11 AA. In another embodiment, the length is 10-11 AA. In another embodiment, the length is 1-2 AA. In another embodiment, the length is 1-3 AA. In another embodiment, the length is 1-4 AA. In another embodiment, the length is 1-5 AA. In another embodiment, the length is 1-6 AA. In another embodiment, the length is 1-7 AA. In another embodiment, the length is 1-8 AA. In another embodiment, the length is 1-9 AA. In another embodiment, the length is 1-10 AA. In another embodiment, the length is 2-3 AA. In another embodiment, the length is 2-4 AA. In another embodiment, the length is 2-5 AA. In another embodiment, the length is 2-6 AA. In another embodiment, the length is 2-7 AA. In another embodiment, the length is 2-8 AA. In another embodiment, the length is 2-9 AA. In another embodiment, the length is 2-10 AA. In another embodiment, the length is 3-4 AA. In another embodiment, the length is 3-5 AA. In another embodiment, the length is 3-6 AA. In another embodiment, the length is 3-7 AA. In another embodiment, the length is 3-8 AA. In another embodiment, the length is 3-9 AA. In another embodiment, the length is 3-10 AA. In another embodiment, the length is 11-50 AA. In another embodiment, the length is 12-50 AA. In another embodiment, the length is 11-15 AA. In another embodiment, the length is 11-20 AA. In another embodiment, the length is 11-25 AA. In another embodiment, the length is 11-30 AA. In another embodiment, the length is 11-35 AA. In another embodiment, the length is 11-40 AA. In another embodiment, the length is 11-60 AA. In another embodiment, the length is 11-70 AA. In another embodiment, the length is 11-80 AA. In another embodiment, the length is 11-90 AA. In another embodiment, the length is 11-100 AA. In another embodiment, the length is 11-150 AA. In another embodiment, the length is 15-20 AA. In another embodiment, the length is 15-25 AA. In another embodiment, the length is 15-30 AA. In another embodiment, the length is 15-35 AA. In another embodiment, the length is 15-40 AA. In another embodiment, the length is 15-60 AA. In another embodiment, the length is 15-70 AA. In another embodiment, the length is 15-80 AA. In another embodiment, the length is 15-90 AA. In another embodiment, the length is 15-100 AA. In another embodiment, the length is 15-150 AA. In another embodiment, the length is 20-25 AA. In another embodiment, the length is 20-30 AA. In another embodiment, the length is 20-35 AA. In another embodiment, the length is 20-40 AA. In another embodiment, the length is 20-60 AA. In another embodiment, the length is 20-70 AA. In another embodiment, the length is 20-80 AA. In another embodiment, the length is 20-90 AA. In another embodiment, the length is 20-100 AA. In another embodiment, the length is 20-150 AA. In another embodiment, the length is 30-35 AA. In another embodiment, the length is 30-40 AA. In another embodiment, the length is 30-60 AA. In another embodiment, the length is 30-70 AA. In another embodiment, the length is 30-80 AA. In another embodiment, the length is 30-90 AA. In another embodiment, the length is 30-100 AA. In another embodiment, the length is 30-150 AA.

In another embodiment, the mutated LLO protein of the present invention that comprises an internal deletion is full length except for the internal deletion. In another embodiment, the mutated LLO protein comprises an additional internal deletion. In another embodiment, the mutated LLO protein comprises more than one additional internal deletion. In another embodiment, the mutated LLO protein is truncated from the C-terminal end.

In another embodiment, the internal deletion of methods and compositions of the present invention comprises the CBD of the mutated LLO protein or fragment thereof. For example, an internal deletion consisting of residues 470-500, 470-510, or 480-500 of SEQ ID NO: 2 comprises the CBD thereof (residues 483-493). In another embodiment, the internal deletion is a fragment of the CBD of the mutated LLO protein or fragment thereof. For example, residues 484-492, 485-490, and 486-488 are all fragments of the CBD of SEQ ID NO: 2. In another embodiment, the internal deletion overlaps the CBD of the mutated LLO protein or fragment thereof. For example, an internal deletion consisting of residues 470-490, 480-488, 490-500, or 486-510 of SEQ ID NO: 2 comprises the CBD thereof.

In another embodiment, a truncated LLO fragment comprises the first 441 AA of the LLO protein. In another embodiment, the LLO fragment comprises the first 420 AA of LLO. In another embodiment, the LLO fragment is a non-hemolytic form of the wild-type LLO protein.

In another embodiment, the LLO fragment consists of about residues 1-25. In another embodiment, the LLO fragment consists of about residues 1-50. In another embodiment, the LLO fragment consists of about residues 1-75. In another embodiment, the LLO fragment consists of about residues 1-100. In another embodiment, the LLO fragment consists of about residues 1-125. In another embodiment, the LLO fragment consists of about residues 1-150. In another embodiment, the LLO fragment consists of about residues 1175. In another embodiment, the LLO fragment consists of about residues 1-200. In another embodiment, the LLO fragment consists of about residues 1-225. In another embodiment, the LLO fragment consists of about residues 1-250. In another embodiment, the LLO fragment consists of about residues 1-275. In another embodiment, the LLO fragment consists of about residues 1-300. In another embodiment, the LLO fragment consists of about residues 1-325. In another embodiment, the LLO fragment consists of about residues 1-350. In another embodiment, the LLO fragment consists of about residues 1-375. In another embodiment, the LLO fragment consists of about residues 1-400. In another embodiment, the LLO fragment consists of about residues 1-425.

In another embodiment, the LLO fragment contains residues of a homologous LLO protein that correspond to one of the above AA ranges. The residue numbers need not, in another embodiment, correspond exactly with the residue numbers enumerated above; e.g. if the homologous LLO protein has an insertion or deletion, relative to an LLO protein utilized herein, then the residue numbers can be adjusted accordingly. In another embodiment, the LLO fragment is any other LLO fragment known in the art.

Methods for identifying corresponding residues of a homologous protein are well known in the art, and include, for example, sequence alignment. In one embodiment, a homologous LLO refers to identity to an LLO sequence (e.g. to one of SEQ ID No: 2-4) of greater than 70%. In another embodiment, a homologous LLO refers to identity to one of SEQ ID No: 2-4 of greater than 72%. In another embodiment, a homologous refers to identity to one of SEQ ID No: 2-4 of greater than 75%. In another embodiment, a homologous refers to identity to one of SEQ ID No: 2-4 of greater than 78%. In another embodiment, a homologous refers to identity to one of SEQ ID No: 2-4 of greater than 80%. In another embodiment, a homologous refers to identity to one of SEQ ID No: 2-4 of greater than 82%. In another embodiment, a homologous refers to identity to one of SEQ ID No: 2-4 of greater than 83%. In another embodiment, a homologous refers to identity to one of SEQ ID No: 2-4 of greater than 85%. In another embodiment, a homologous refers to identity to one of SEQ ID No: 2-4 of greater than 87%. In another embodiment, a homologous refers to identity to one of SEQ ID No: 2-4 of greater than 88%. In another embodiment, a homologous refers to identity to one of SEQ ID No: 2-4 of greater than 90%. In another embodiment, a homologous refers to identity to one of SEQ ID No: 2-4 of greater than 92%. In another embodiment, a homologous refers to identity to one of SEQ ID No: 2-4 of greater than 93%. In another embodiment, a homologous refers to identity to one of SEQ ID No: 2-4 of greater than 95%. In another embodiment, a homologous refers to identity to one of SEQ ID No: 2-4 of greater than 96%. In another embodiment, a homologous refers to identity to one of SEQ ID No: 2-4 of greater than 97%. In another embodiment, a homologous refers to identity to one of SEQ ID No: 2-4 of greater than 98%. In another embodiment, a homologous refers to identity to one of SEQ ID No: 2-4 of greater than 99%. In another embodiment, a homologous refers to identity to one of SEQ ID No: 2-4 of 100%.

The terms “PEST amino acid sequence,” “PEST sequence,” “PEST sequence peptide,” “PEST peptide,” or “PEST sequence-containing protein or peptide,” are used interchangeably herein. It will be appreciated by the skilled artisan that these terms may encompass a truncated LLO protein, which in one embodiment is an N-terminal LLO, or in another embodiment, a truncated ActA protein. PEST sequence peptides are known in the art and are described in U.S. Pat. No. 7,635,479, and in US Patent Publication Serial No. 2014/0186387, both of which are hereby incorporated in their entirety herein.

In another embodiment, a PEST sequence of prokaryotic organisms can be identified routinely in accordance with methods such as described by Rechsteiner and Roberts (TBS 21:267-271, 1996) for L. monocytogenes. Alternatively, PEST amino acid sequences from other prokaryotic organisms can also be identified based by this method. Other prokaryotic organisms wherein PEST amino acid sequences would be expected to include, but are not limited to, other Listeria species. For example, the L. monocytogenes protein ActA contains four such sequences. These are KTEEQPSEVNTGPR (SEQ ID NO: 5), KASVTDTSEGDLDSSMQSADESTPQPLK (SEQ ID NO: 6), KNEEVNASDFPPPPTDEELR (SEQ ID NO: 7), and RGGIPTSEEFSSLNSGDFTDDENSETTEEEIDR (SEQ ID NO: 8). Also Streptolysin O from Streptococcus sp. contain a PEST sequence. For example, Streptococcus pyogenes Streptolysin O comprises the PEST sequence KQNTASTETTTTNEQPK (SEQ ID NO: 9) at amino acids 35-51 and Streptococcus equisimilis Streptolysin O comprises the PEST-like sequence KQNTANTETTTTNEQPK (SEQ ID NO: 10) at amino acids 38-54. Further, it is believed that the PEST sequence can be embedded within the antigenic protein. Thus, for purposes of the present invention, by “fusion” when in relation to PEST sequence fusions, it is meant that the antigenic protein comprises both the antigen and the PEST amino acid sequence either linked at one end of the antigen or embedded within the antigen. In other embodiments, a PEST sequence or PEST containing polypeptide is not part of a fusion protein, nor does the polypeptide include a heterologous antigen.

The terms “nucleic acid sequence,” “nucleic acid molecule,” “polynucleotide,” or “nucleic acid construct” are used interchangeably herein, and may refer to a DNA or RNA molecule, which may include, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also refers to sequences that include any of the known base analogs of DNA and RNA. The terms may also refer to a string of at least two base-sugar-phosphate combinations. The term may also refer to the monomeric units of nucleic acid polymers. RNA may be, in one embodiment, in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al, Genes & Devel 16: 2491-96 and references cited therein). DNA may be in form of plasmid DNA, viral DNA, linear DNA, or chromosomal DNA or derivatives of these groups. In addition, these forms of DNA and RNA may be single, double, triple, or quadruple stranded. The terms may also include, artificial nucleic acids that may contain other types of backbones but the same bases. In one embodiment, the artificial nucleic acid is a PNA (peptide nucleic acid). PNA contain peptide backbones and nucleotide bases and are able to bind, in one embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. The use of phosphothiorate nucleic acids and PNA are known to those skilled in the art, and are described in, for example, Neilsen P E, Curr Opin Struct Biol 9:353-57; and Raz N K et al Biochem Biophys Res Commun. 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed.

In another embodiment, a nucleic acid molecule provided herein is expressed from an episomal or plasmid vector. In another embodiment, the plasmid is stably maintained in the recombinant Listeria immunotherapy strain in the absence of antibiotic selection. In another embodiment, the plasmid does not confer antibiotic resistance upon the recombinant Listeria.

In one embodiment, an immunogenic polypeptide or fragment thereof provided herein is an ActA protein or fragment thereof. In one embodiment, an ActA protein comprises the sequence set forth in SEQ ID NO: 11:

(SEQ ID NO: 11)
MRAMMVVFITANCITINPDIIFAATDSEDSSLNTDEWEEEKTEEQPSEVN
TGPRYETAREVSSRDIEELEKSNKVKNTNKADLIAMLKAKAEKGPNNNNN
NGEQTGNVAINEEASGVDRPTLQVERRHPGLSSDSAAEIKKRRKAIASSD
SELESLTYPDKPTKANKRKVAKESVVDASESDLDSSMQSADESTPQPLKA
NQKPFFPKVFKKIKDAGKWVRDKIDENPEVKKAIVDKSAGLIDQLLTKKK
SEEVNASDFPPPPTDEELRLALPETPMLLGFNAPTPSEPSSFEFPPPPTD
EELRLALPETPMLLGFNAPATSEPSSPEFPPPPTEDELEIMRETAPSLDS
SFTSGDLASLRSAINRHSENFSDFPLIPTEEELNGRGGRPTSEEFSSLNS
GDFTDDENSETTEEEIDRLADLRDRGTGKHSRNAGFLPLNPFISSPVPSL
TPKVPKISAPALISDITKKAPFKNPSQPLNVFNKKTTTKTVTKKPTPVKT
APKLAELPATKPQETVLRENKTPFIEKQAETNKQSINMPSLPVIQKEATE
SDKEEMKPQTEEKMVEESESANNANGKNRSAGIEEGKLIAKSAEDEKAKE
EPGNHTTLILAMLAIGVFSLGAFIKIIQLRKNN.

The first 29 AA of the proprotein corresponding to this sequence are the signal sequence and are cleaved from ActA protein when it is secreted by the bacterium. In one embodiment, an ActA polypeptide or peptide comprises the signal sequence, AA 1-29 of SEQ ID NO: 11 above. In another embodiment, an ActA polypeptide or peptide does not include the signal sequence, AA 1-29 of SEQ ID NO: 11 above.

In one embodiment, a truncated ActA protein comprises an N-terminal fragment of an ActA protein. In another embodiment, a truncated ActA protein is an N-terminal fragment of an ActA protein. In one embodiment, a truncated ActA protein comprises the sequence set forth in SEQ ID NO: 12:

(SEQ ID NO: 12)
MRAMMVVFITANCITINPDIIFAATDSEDSSLNTDEWEEEKTEEQPSEVN
TGPRYETAREVSSRDIKELEKSNKVRNTNKADLIAMLKEKAEKGPNINNN
NSEQTENAAINEEASGADRPAIQVERRHPGLPSDSAAEIKKRRKAIASSD
SELESLTYPDKPTKVNKKKVAKESVADASESDLDSSMQSADESSPQPLKA
NQQPFFPKVFKKIKDAGKWVRDKIDENPEVKKAIVDKSAGLIDQLLTKKK
SEEVNASDFPPPPTDEELRLALPETPMLLGFNAPATSEPSSFEFPPPPTD
EELRLALPETPMLLGFNAPATSEPSSPEFPPPPTEDELEIIRETASSLDS
SFTRGDLASLRNAINRHSQNFSDFPPIPTEEELNGRGGRP.

In another embodiment, the ActA fragment comprises the sequence set forth in SEQ ID NO: 12.

In another embodiment, a truncated ActA protein comprises the sequence set forth in SEQ ID NO: 13: MGLNRFMRAMMVVFITANCITINPDIIFAATDSEDSSLNTDEWEEEKTEEQPSEVNTG PRYETAREVSSRDIKELEKSNKVRNTNKADLIAMLKEKAEKG (SEQ ID NO: 13).

In another embodiment, the ActA fragment is any other ActA fragment known in the art. In another embodiment, the ActA fragment is an immunogenic fragment.

In another embodiment, an ActA protein comprises the sequence set forth in SEQ ID NO: 14: M G L N R F M R A M M V V F I T A N C I T I N P D I I F A A T D S E D S S L N T D E W E E E K T E E Q P S E V N T G P R Y E T A R E V S S R D I E E L E K S N K V K N T N K A D L I A M L K A K A E K G P N N N N N N G E Q T G N V A I N E E A S G V D R P T L Q V E R R H P G L S S D S A A E I K K R R K A I A S S D S E L E S L T Y P D K P T K A N K R K V A K E S V V D A S E S D L D S S M Q S A D E S T P Q P L K A N Q K P F F P K V F K K I K D A G K W V R D K I D E N P E V K K A I V D K S A G L I D Q L L T K K K S E E V N A S D F P P P P T D E E L R L A L P E T P M L L G F N A P T P S E P S S F E F P P P P T D E E L R L A L P E T P M L L G F N A P A T S E P S S F E F P P P P T E D E L E I M R E T A P S L D S S F T S G D L A S L R S A I N R H S E N F S D F P L I P T E E E L N G R G G R P T S E E F S S L N S G D F T D D E N S E T T E E E I D R L A D L R D R G T G K H S R N A G F L P L N P F I S S P V P S L T P K V P K I S A P A L I S D I T K K A P F K N P S Q P L N V F N K K T T T K T V T K K P T P V K T A P K L A E L P A T K P Q E T V L R E N K T P F I E K Q A E T N K Q S I N M P S L P V I Q K E A T E S D K E E M K P Q T E E K M V E E S E S A N N A N G K N R S A G I E E G K L I A K S A E D E K A K E E P G N H T T L I L A M L A I G V F S L G A F I K I I Q L R K N N (SEQ ID NO: 14). The first 29 AA of the proprotein corresponding to this sequence are the signal sequence and are cleaved from ActA protein when it is secreted by the bacterium. In one embodiment, an ActA polypeptide or peptide comprises the signal sequence, AA 1-29 of SEQ ID NO: 154. In another embodiment, an ActA polypeptide or peptide does not include the signal sequence, AA 1-29 of SEQ ID NO: 14.

In another embodiment, a truncated ActA protein comprises the sequence set forth in SEQ ID NO: 15:

A T D S E D S S L N T D E W E E E K T E E Q P S E V N T G P R Y E T A R E V S S R D I E E L E K S N K V K N T N K A D L I A M L K A K A E K G P N N N N N N G E Q T G N V A I N E E A S G (SEQ ID NO: 15). In another embodiment, a truncated ActA as set forth in SEQ ID NO: 15 is referred to as ActA/PEST1. In another embodiment, a truncated ActA comprises from the first 30 to amino acid 122 of the full length ActA sequence. In another embodiment, SEQ ID NO: 15 comprises from the first 30 to amino acid 122 of the full length ActA sequence. In another embodiment, a truncated ActA comprises from the first 30 to amino acid 122 of SEQ ID NO: 14. In another embodiment, SEQ ID NO: 15 comprises from the first 30 to amino acid 122 of SEQ ID NO: 14.

In another embodiment, a truncated ActA protein comprises the sequence set forth in SEQ ID NO: 16:

A T D S E D S S L N T D E W E E E K T E E Q P S E V N T G P R Y E T A R E V S S R D I E E L E K S N K V K N T N K A D L I A M L K A K A E K G P N N N N N N G E Q T G N V A I N E E A S G V D R P T L Q V E R R H P G L S S D S A A E I K K R R K A I A S S D S E L E S L T Y P D K P T K A N K R K V A K E S V V D A S E S D L D S S M Q S A D E S T P Q P L K A N Q K P F F P K V F K K I K D A G K W V R D K (SEQ ID NO: 16). In another embodiment, a truncated ActA as set forth in SEQ ID NO: 16 is referred to as ActA/PEST2. In another embodiment, a truncated ActA as set forth in SEQ ID NO: 16 is referred to as LA229. In another embodiment, a truncated ActA comprises from amino acid 30 to amino acid 229 of the full length ActA sequence. In another embodiment, SEQ ID NO: 16 comprises from about amino acid 30 to about amino acid 229 of the full length ActA sequence. In another embodiment, a truncated ActA comprises from about amino acid 30 to amino acid 229 of SEQ ID NO: 14. In another embodiment, SEQ ID NO: 16 comprises from amino acid 30 to amino acid 229 of SEQ ID NO: 14.

In another embodiment, a truncated ActA sequence disclosed herein is further fused to an hly signal peptide at the N-terminus. In another embodiment, the truncated ActA fused to hly signal peptide comprises SEQ ID NO: 138:

MKKIMLVFITLILVSLPIAQQTEASRATDSEDSS
LNTDEWEEEKTEEQPSEVNTGPRYETAREVSSR
DIEELEKSNKVKNTNKADLIAMLKAKAEKGPN
NNNNNGEQTGNVAINEEASGVDRPTLQVERRH
PGLSSDSAAEIKKRRKAIASSDSELESLTYPDKP
TKANKRKVAKESVVDASESDLDSSMQSADEST
PQPLKANQKPFFPKVFKKIKDAGKWVRDK.

In another embodiment, a truncated ActA fused to hly signal peptide is encoded by a sequence comprising SEQ ID NO: 139: Atgaaaaaaataatgctagtttttattacacttatattagttagtctaccaattgcgcaacaaactgaagcatctagagcgacagatagcg aagattccagtctaaacacagatgaatgggaagaagaaaaaacagaagagcagccaagcgaggtaaatacgggaccaagatacga aactgcacgtgaagtaagttcacgtgatattgaggaactagaaaaatcgaataaagtgaaaaatacgaacaaagcagacctaatagca atgttgaaagcaaaagcagagaaaggtccgaataacaataataacaacggtgagcaaacaggaaatgtggctataaatgaagaggct tcaggagtcgaccgaccaactctgcaagtggagcgtcgtcatccaggtctgtcatcggatagcgcagcggaaattaaaaaaagaaga aaagccatagcgtcgtcggatagtgagcttgaaagccttacttatccagataaaccaacaaaagcaaataagagaaaagtggcgaaag agtcagttgtggatgcttctgaaagtgacttagattctagcatgcagtcagcagacgagtctacaccacaacctttaaaagcaaatcaaa aaccatttttccctaaagtatttaaaaaaataaaagatgcggggaaatgggtacgtgataaa (SEQ ID NO: 139). In another embodiment, SEQ ID NO: 139 comprises a sequence encoding a linker region (see bold, italic text) that is used to create a unique restriction enzyme site for XbaI so that different polypeptides, heterologous antigens, etc. can be cloned after the signal sequence. Hence, it will be appreciated by a skilled artisan that signal peptidases act on the sequences before the linker region to cleave signal peptide.

In another embodiment, a truncated ActA protein comprises the sequence set forth in SEQ ID NO: 17: A T D S E D S S L N T D E W E E E K T E E Q P S E V N T G P R Y E T A R E V S S R D I E E L E K S N K V K N T N K A D L I A M L K A K A E K G P N N N N N N G E Q T G N V A I N E E A S G V D R P T L Q V E R R H P G L S S D S A A E I K K R R K A I A S S D S E L E S L T Y P D K P T K A N K R K V A K E S V V D A S E S D L D S S M Q S A D E S T P Q P L K A N Q K P F F P K V F K K I K D A G K W V R D K I D E N P E V K K A I V D K S A G L I D Q L L T K K K S E E V N A S D F P P P P T D E E L R L A L P E T P M L L G F N A P T P S E P S S F E F P P P P T D E E L R L A L P E T P M L L G F N A P A T S E P S S (SEQ ID NO: 17). In another embodiment, a truncated ActA as set forth in SEQ ID NO: 17 is referred to as ActA/PEST3. In another embodiment, this truncated ActA comprises from the first 30 to amino acid 332 of the full length ActA sequence. In another embodiment, SEQ ID NO: 17 comprises from the first 30 to amino acid 332 of the full length ActA sequence. In another embodiment, a truncated ActA comprises from about the first 30 to amino acid 332 of SEQ ID NO: 14. In another embodiment, SEQ ID NO: 17 comprises from the first 30 to amino acid 332 of SEQ ID NO: 14.

In another embodiment, a truncated ActA protein comprises the sequence set forth in SEQ ID NO: 18:

(SEQ ID NO: 18)
ATDSEDSSLNTDEWEEEKTEEQPSEVNTGPRYETAREVSSRD
IEELEKSNKVKNTNKADLIAMLKAKAEKGPNNNNNNGEQTG
NVAINEEASGVDRPTLQVERRHPGLSSDSAAEIKKRRKAIAS
SDSELESLTYPDKPTKANKRKVAKESVVDASESDLDSSMQS
ADESTPQPLKANQKPFFPKVFKKIKDAGKWVRDKIDENPEV
KKAIVDKSAGLIDQLLTKKKSEEVNASDFPPPPTDEELRLAL
PETPMLLGFNAPTPSEPSSFEFPPPPTDEELRLALPETPMLLG
FNAPATSEPSSFEFPPPPTEDELEIMRETAPSLDSSFTSGDLA
SLRSAINRHSENFSDFPLIPTEEELNGRGGRPTSE.

In another embodiment, a truncated ActA as set forth in SEQ ID NO: 18 is referred to as ActA/PEST4. In another embodiment, this truncated ActA comprises from the first 30 to amino acid 399 of the full length ActA sequence. In another embodiment, SEQ ID NO: 18 comprises from the first 30 to amino acid 399 of the full length ActA sequence. In another embodiment, a truncated ActA comprises from the first 30 to amino acid 399 of SEQ ID NO: 14. In another embodiment, SEQ ID NO: 18 comprises from the first 30 to amino acid 399 of SEQ ID NO: 14.

In another embodiment, “truncated ActA” or “AActA” refers to a fragment of ActA that comprises a PEST domain. In another embodiment, the terms refer to an ActA fragment that comprises a PEST sequence.

In another embodiment, the recombinant nucleotide encoding a truncated ActA protein comprises the sequence set forth in SEQ ID NO: 19:

atgcgtgcgatgatggtggttttcattactgccaattgcattacgattaa
ccccgacataatatttgcagcgacagatagcgaagattctagtctaaaca
cagatgaatgggaagaagaaaaaacagaagagcaaccaagcgaggtaaat
acgggaccaagatacgaaactgcacgtgaagtaagttcacgtgatattaa
agaactagaaaaatcgaataaagtgagaaatacgaacaaagcagacctaa
tagcaatgttgaaagaaaaagcagaaaaaggtccaaatatcaataataac
aacagtgaacaaactgagaatgcggctataaatgaagaggcttcaggagc
cgaccgaccagctatacaagtggagcgtcgtcatccaggattgccatcgg
atagcgcagcggaaattaaaaaaagaaggaaagccatagcatcatcggat
agtgagcttgaaagccttacttatccggataaaccaacaaaagtaaataa
gaaaaaagtggcgaaagagtcagttgcggatgcttctgaaagtgacttag
attctagcatgcagtcagcagatgagtcacaccacaacctttaaaagcaa
accaacaaccatttttccctaaagtatttaaaaaaataaaagatgcgggg
aaatgggtacgtgataaaatcgacgaaaatcctgaagtaaagaaagcgat
tgttgataaaagtgcagggttaattgaccaattattaaccaaaaagaaaa
gtgaagaggtaaatgcttcggacttcccgccaccacctacggatgaagag
ttaagacttgctttgccagagacaccaatgcttcttggttttaatgctcc
tgctacatcagaaccgagctcattcgaatttccaccaccacctacggatg
aagagttaagacttgctttgccagagacgccaatgcttcttggttttaat
gctcctgctacatcggaaccgagctcgttcgaatttccaccgcctccaac
agaagatgaactagaaatcatccgggaaacagcatcctcgctagattcta
gttttacaagaggggatttagctagtttgagaaatgctattaatcgccat
agtcaaaatttctctgatttcccaccaatcccaacagaagaagagttgaa
cgggagaggcggtagacca.

In another embodiment, the recombinant nucleotide has the sequence set forth in SEQ ID NO: 19. In another embodiment, the recombinant nucleotide comprises any other sequence that encodes a fragment of an ActA protein.

In another embodiment, the ActA fragment consists of about the first 100 AA of the ActA protein.

In another embodiment, the ActA fragment consists of about residues 1-25. In another embodiment, the ActA fragment consists of about residues 1-50. In another embodiment, the ActA fragment consists of about residues 1-75. In another embodiment, the ActA fragment consists of about residues 1-100. In another embodiment, the ActA fragment consists of about residues 1-125. In another embodiment, the ActA fragment consists of about residues 1-150. In another embodiment, the ActA fragment consists of about residues 1-175. In another embodiment, the ActA fragment consists of about residues 1-200. In another embodiment, the ActA fragment consists of about residues 1-225. In another embodiment, the ActA fragment consists of about residues 1-250. In another embodiment, the ActA fragment consists of about residues 1-275. In another embodiment, the ActA fragment consists of about residues 1-300. In another embodiment, the ActA fragment consists of about residues 1-325. In another embodiment, the ActA fragment consists of about residues 1-338. In another embodiment, the ActA fragment consists of about residues 1-350. In another embodiment, the ActA fragment consists of about residues 1-375. In another embodiment, the ActA fragment consists of about residues 1-400. In another embodiment, the ActA fragment consists of about residues 1-450. In another embodiment, the ActA fragment consists of about residues 1-500. In another embodiment, the ActA fragment consists of about residues 1-550. In another embodiment, the ActA fragment consists of about residues 1-600. In another embodiment, the ActA fragment consists of about residues 1-639. In another embodiment, the ActA fragment consists of about residues 30-100. In another embodiment, the ActA fragment consists of about residues 30-125. In another embodiment, the ActA fragment consists of about residues 30-150. In another embodiment, the ActA fragment consists of about residues 30-175. In another embodiment, the ActA fragment consists of about residues 30-200. In another embodiment, the ActA fragment consists of about residues 30-225. In another embodiment, the ActA fragment consists of about residues 30-250. In another embodiment, the ActA fragment consists of about residues 30-275. In another embodiment, the ActA fragment consists of about residues 30-300. In another embodiment, the ActA fragment consists of about residues 30-325. In another embodiment, the ActA fragment consists of about residues 30-338. In another embodiment, the ActA fragment consists of about residues 30-350. In another embodiment, the ActA fragment consists of about residues 30-375. In another embodiment, the ActA fragment consists of about residues 30-400. In another embodiment, the ActA fragment consists of about residues 30-450. In another embodiment, the ActA fragment consists of about residues 30-500. In another embodiment, the ActA fragment consists of about residues 30-550. In another embodiment, the ActA fragment consists of about residues 1-600. In another embodiment, the ActA fragment consists of about residues 30-604.

In another embodiment, the ActA fragment contains residues of a homologous ActA protein that correspond to one of the above AA ranges. The residue numbers need not, in another embodiment, correspond exactly with the residue numbers enumerated above; e.g. if the homologous ActA protein has an insertion or deletion, relative to an ActA protein utilized herein, then the residue numbers can be adjusted accordingly. In another embodiment, the ActA fragment is any other ActA fragment known in the art.

In another embodiment, a homologous ActA refers to identity to an ActA sequence (e.g. to one of SEQ ID No: 11-18) of greater than 70%. In another embodiment, a homologous ActA refers to identity to one of SEQ ID No: 11-18 of greater than 72%. In another embodiment, a homologous refers to identity to one of SEQ ID No: 11-18 of greater than 75%. In another embodiment, a homologous refers to identity to one of SEQ ID No: 11-18 of greater than 78%. In another embodiment, a homologous refers to identity to one of SEQ ID No: 11-18 of greater than 80%. In another embodiment, a homologous refers to identity to one of SEQ ID No: 11-18 of greater than 82%. In another embodiment, a homologous refers to identity to one of SEQ ID No: 11-18 of greater than 83%. In another embodiment, a homologous refers to identity to one of SEQ ID No: 11-18 of greater than 85%. In another embodiment, a homologous refers to identity to one of SEQ ID No: 11-18 of greater than 87%. In another embodiment, a homologous refers to identity to one of SEQ ID No: 11-18 of greater than 88%. In another embodiment, a homologous refers to identity to one of SEQ ID No: 11-18 greater than 90%. In another embodiment, a homologous refers to identity to one of SEQ ID No: 11-18 of greater than 92%. In another embodiment, a homologous refers to identity to one of SEQ ID No: 11-18 of greater than 93%. In another embodiment, a homologous refers to identity to one of SEQ ID No: 11-18 of greater than 95%. In another embodiment, a homologous refers to identity to one of SEQ ID No: 11-18 of greater than 96%. In another embodiment, a homologous refers to identity to one of SEQ ID No: 11-18 of greater than 97%. In another embodiment, a homologous refers to identity to one of SEQ ID No: 11-18 of greater than 98%. In another embodiment, a homologous refers to identity to one of SEQ ID No: 11-18 of greater than 99%. In another embodiment, a homologous refers to identity to one of SEQ ID No: 11-18 of 100%.

It will be appreciated by the skilled artisan that the term “homology,” when in reference to any nucleic acid sequence provided herein may encompass a percentage of nucleotides in a candidate sequence that is identical with the nucleotides of a corresponding native nucleic acid sequence.

Homology is, in one embodiment, determined by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

In another embodiment, “homology” refers to identity to a sequence selected from the sequences provided herein of greater than 68%. In another embodiment, “homology” refers to identity to a sequence selected from the sequences provided herein of greater than 70%. In another embodiment, “homology” refers to identity to a sequence selected from the sequences provided herein of greater than 72%. In another embodiment, the identity is greater than 75%. In another embodiment, the identity is greater than 78%. In another embodiment, the identity is greater than 80%. In another embodiment, the identity is greater than 82%. In another embodiment, the identity is greater than 83%. In another embodiment, the identity is greater than 85%. In another embodiment, the identity is greater than 87%. In another embodiment, the identity is greater than 88%. In another embodiment, the identity is greater than 90%. In another embodiment, the identity is greater than 92%. In another embodiment, the identity is greater than 93%. In another embodiment, the identity is greater than 95%. In another embodiment, the identity is greater than 96%. In another embodiment, the identity is greater than 97%. In another embodiment, the identity is greater than 98%. In another embodiment, the identity is greater than 99%. In another embodiment, the identity is 100%.

In another embodiment, homology is determined via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y). For example methods of hybridization may be carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA.

In one embodiment, the recombinant Listeria strain provided herein lacks antibiotic resistance genes.

In one embodiment, the recombinant Listeria provided herein is capable of escaping the phagolysosome.

In one embodiment, the Listeria genome comprises a deletion of the endogenous actA gene, which in one embodiment is a virulence factor. In one embodiment, the heterologous antigen or antigenic polypeptide is integrated in frame with LLO in the Listeria chromosome. In another embodiment, the integrated nucleic acid molecule is integrated in frame with ActA into the actA locus. In another embodiment, the chromosomal nucleic acid encoding ActA is replaced by a nucleic acid molecule encoding an antigen.

In one embodiment, a peptide provided herein comprises one or more neo-epitopes. In one embodiment, a peptide provided herein is comprised by an antigen. In another embodiment, a peptide provided herein is an antigen fragment. In one embodiment, an antigen provided herein comprises one or more neo-epitopes. In another embodiment, the antigen is a heterologous antigen or a self-antigen. In one embodiment, a heterologous antigen or self-antigen provided herein is a tumor-associated antigen. It will be appreciated by a skilled artisan that the term “heterologous” may refer to an antigen, or portion thereof, which is not naturally or normally expressed from a bacterium. In one embodiment, a heterologous antigen comprises an antigen not naturally or normally expressed from a Listeria strain. In another embodiment, the tumor-associated antigen is a naturally occurring tumor-associated antigen. In another embodiment, the tumor-associated antigen is a synthetic tumor-associated antigen. In yet another embodiment, the tumor-associated antigen is a chimeric tumor-associated antigen. In still another embodiment, the tumor-associated antigen comprises one or more neo-epitopes. In still another embodiment, the tumor-associated antigen is a neo-antigen.

In one embodiment, a recombinant Listeria provided herein comprises a nucleic acid molecule comprising a first open reading frame encoding recombinant polypeptide comprising one or more peptides, wherein said one or more peptides comprise one or more neo-epitopes. In another embodiment, the recombinant polypeptide further comprises a truncated LLO protein, a truncated ActA protein or PEST sequence fused to a peptide provided herein.

In another embodiment, the nucleic acid molecule provided herein comprises a first open reading frame encoding a recombinant polypeptide comprising a truncated LLO protein, a truncated ActA protein or a PEST sequence, wherein the truncated LLO protein, a truncated ActA protein or a PEST sequence peptide is not fused to a heterologous antigen. In another embodiment, the first open reading frame encodes a truncated LLO protein. In another embodiment, the first open reading frame encodes a truncated ActA protein. In another embodiment, the first open reading frame encodes a truncated LLO protein. In another embodiment, the first open reading frame encodes a truncated ActA protein. In another embodiment, the first open reading frame encodes a truncated LLO protein. In another embodiment, the first open reading frame encodes a truncated ActA protein consisting of an N-terminal ActA protein or fragment thereof.

It will be appreciated by a skilled artisan that the terms “antigen,” “antigen fragment,” “antigen portion,” “heterologous protein,” “heterologous protein antigen,” “protein antigen,” “antigen,” “antigenic polypeptide,” or their grammatical equivalents, which are used interchangeably herein, may refer to a polypeptide, peptide or recombinant peptide as described herein that is processed and presented on MHC class I and/or class II molecules present in a subject's cells leading to the mounting of an immune response when present in, or in another embodiment, detected by, the host. In one embodiment, the antigen may be foreign to the host. In another embodiment, the antigen might be present in the host but the host does not elicit an immune response against it because of immunologic tolerance. In another embodiment, the antigen is a neo-antigen comprising one or more neo-epitopes, wherein one or more neo-epitopes are T-cell epitopes. In another embodiment, the antigen or a peptide fragment thereof comprises one or more neo-epitopes that are T-cell epitopes.

In another embodiment, an antigen comprises at least one neo-epitope. In one embodiment, an antigen is a neo-antigen comprising at least one neo-epitope. In one embodiment, a neo-epitope is an epitope that has not been previously recognized by the immune system. Neo-antigens are often associated with tumor antigens and are found in oncogenic cells. Neo-antigens and, by extension, neo-antigenic determinants (neo-epitopes) may be formed when a protein undergoes further modification within a biochemical pathway such as glycosylation, phosphorylation or proteolysis. This, by altering the structure of the protein, can produce new or “neo” epitopes.

In one embodiment, a Listeria provided herein comprises a minigene nucleic acid construct, said construct comprising one or more open reading frames encoding a chimeric protein, wherein said chimeric protein comprises:
a. a bacterial secretion signal sequence;
b. a ubiquitin (Ub) protein;
c. one or more peptides comprising said one or more neo-epitopes; and,
   wherein said signal sequence, said ubiquitin and said one or more peptides in a.-c. are respectively arranged in tandem, or are operatively linked, from the amino-terminus to the carboxy-terminus.

In another embodiment, a bacterial signal sequence provided herein is a Listerial signal sequences, which in another embodiment is an hly or an actA signal sequence. In another embodiment, the bacterial signal sequence is any other signal sequence known in the art. In another embodiment, a recombinant Listeria comprising a minigene nucleic acid construct further comprises two or more open reading frames linked by a Shine-Dalgarno ribosome binding site nucleic acid sequence between each open reading frame. In another embodiment, a recombinant Listeria comprising a minigene nucleic acid construct further comprises one to four open reading frames linked by a Shine-Dalgarno ribosome binding site nucleic acid sequence between each open reading frame. In another embodiment, each open reading frame encodes a different peptide.

In another embodiment, provided herein is a recombinant attenuated Listeria strain comprising a recombinant nucleic acid construct comprising an open reading frame encoding a bacterial secretion signal sequence (SS), a ubiquitin (Ub) protein, and a peptide sequence. In another embodiment, the nucleic acid construct encodes a chimeric protein comprising a bacterial secretion signal sequence, a ubiquitin protein, and a peptide sequence. In one embodiment, the chimeric protein is arranged in the following manner (SS-Ub-Peptide).

In one embodiment, the nucleic acid construct comprises a codon that corresponds to the carboxy-terminus of the peptide moiety is followed by two stop codons to ensure termination of protein synthesis.

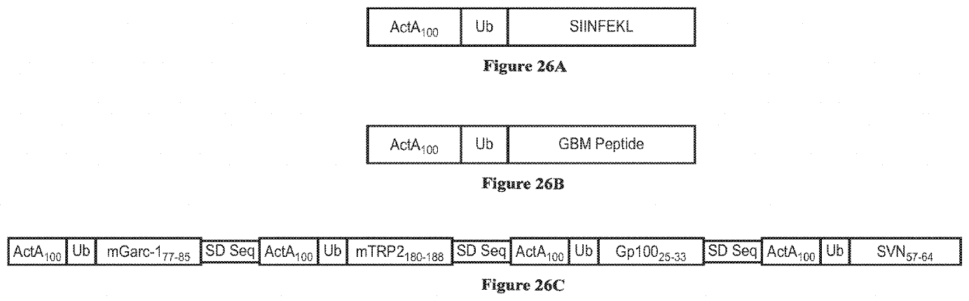

In one embodiment, a minigene nucleic acid construct provided in the compositions and methods described herein comprises an expression system that is designed to facilitate panels of recombinant proteins containing distinct peptide moieties at the carboxy terminus. This is accomplished, in one embodiment, by a PCR reaction utilizing a sequence encoding one of the bacterial secretion signal sequence-ubiquitin-peptide (SS-Ub-Peptide) constructs as a template. In one embodiment, using a primer that extends into the carboxy-terminal region of the Ub sequence and introducing codons for the desired peptide sequence at the 3′ end of the primer, a new SS-Ub-Peptide sequence can be generated in a single PCR reaction (see Examples herein). The 5′ primer encoding the bacterial promoter and the first few nucleotides of the bacterial secretion signal sequence may be the same for all the constructs. A schematic representation of this construct is provided in FIG. 26A-C herein.

In one embodiment, nucleic acids encoding recombinant polypeptides provided herein also comprise a signal peptide or signal sequence. In one embodiment, the bacterial secretion signal sequence encoded by a nucleic acid constructs or nucleic acid molecule provided herein is a Listeria secretion signal sequence. In another embodiment, a fusion protein of methods and compositions of the present invention comprises an LLO signal sequence from Listeriolysin O (LLO). It will be appreciated by a skilled artisan that an antigen or a peptide comprising one or more neo-epitopes provided herein may be expressed through the use of a signal sequence, such as a Listerial signal sequence, for example, the hemolysin (hly) signal sequence or the actA signal sequence. Alternatively, for example, foreign genes can be expressed downstream from a L. monocytogenes promoter without creating a fusion protein. In another embodiment, the signal peptide is bacterial (Listerial or non-Listerial). In one embodiment, the signal peptide is native to the bacterium. In another embodiment, the signal peptide is foreign to the bacterium. In another embodiment, the signal peptide is a signal peptide from Listeria monocytogenes, such as a secA1 signal peptide. In another embodiment, the signal peptide is an Usp45 signal peptide from Lactococcus lactis, or a Protective Antigen signal peptide from Bacillus anthracis. In another embodiment, the signal peptide is a secA2 signal peptide, such the p60 signal peptide from Listeria monocytogenes. In addition, the recombinant nucleic acid molecule optionally comprises a third polynucleotide sequence encoding p60, or a fragment thereof. In another embodiment, the signal peptide is a Tat signal peptide, such as a B. subtilis Tat signal peptide (e.g., PhoD). In one embodiment, the signal peptide is in the same translational reading frame encoding the recombinant polypeptide.

In another embodiment, the secretion signal sequence is from a Listeria protein. In another embodiment, the secretion signal is an ActA₃₀₀ secretion signal. In another embodiment, the secretion signal is an ActA₁₀₀ secretion signal.

In one embodiment, the nucleic acid construct comprises an open reading frame encoding a ubiquitin protein. In one embodiment, the ubiquitin is a full-length protein. It will be appreciated by the skilled artisan that the Ubiquitin in the expressed construct provided herein (expressed from the nucleic acid construct provided herein) is cleaved at the carboxy-terminus from the rest of the recombinant chimeric protein expressed from the nucleic acid construct through the action of hydrolases upon entry to the host cell cytosol. This liberates the amino-terminus of the peptide moiety, producing a peptide (length depends on the specific peptide) in the host cell cytosol.

In one embodiment, the peptide encoded by the nucleic acid constructs provided herein is 8-10 amino acids (AA) in length. In another embodiment, the peptide is 10-20 AA long. In another embodiment, the peptide is a 21-30 AA long. In another embodiment, the peptide is 31-50 AA long. In another embodiment, the peptide is 51-100 AA long.

In one embodiment, a nucleic acid molecule provided herein further comprises a second open reading frame encoding a metabolic enzyme. In another embodiment, the metabolic enzyme complements an endogenous gene that is lacking in the chromosome of the recombinant Listeria strain. In another embodiment, the metabolic enzyme complements an endogenous gene that is mutated in the chromosome of the recombinant Listeria strain. In another embodiment, the metabolic enzyme encoded by the second open reading frame is an alanine racemase enzyme (dal). In another embodiment, the metabolic enzyme encoded by the second open reading frame is a D-amino acid transferase enzyme (dat). In another embodiment, the Listeria strains provided herein comprise a mutation in the endogenous dal/dat genes. In another embodiment, the Listeria lacks the dal/dat genes.

In another embodiment, a nucleic acid molecule of the methods and compositions of the present invention is operably linked to a promoter/regulatory sequence. In another embodiment, the first open reading frame of methods and compositions of the present invention is operably linked to a promoter/regulatory sequence. In another embodiment, the second open reading frame of methods and compositions of the present invention is operably linked to a promoter/regulatory sequence. In another embodiment, each of the open reading frames are operably linked to a promoter/regulatory sequence.

“Metabolic enzyme” refers, in another embodiment, to an enzyme involved in synthesis of a nutrient required by the host bacteria. In another embodiment, the term refers to an enzyme required for synthesis of a nutrient required by the host bacteria. In another embodiment, the term refers to an enzyme involved in synthesis of a nutrient utilized by the host bacteria. In another embodiment, the term refers to an enzyme involved in synthesis of a nutrient required for sustained growth of the host bacteria. In another embodiment, the enzyme is required for synthesis of the nutrient.

In another embodiment, the recombinant Listeria is an attenuated auxotrophic strain. In another embodiment, the recombinant Listeria is an Lm-LLO-E7 strain described in U.S. Pat. No. 8,114,414, which is incorporated by reference herein in its entirety.

In one embodiment the attenuated strain is Lm dal(−)dat(−) (Lmdd). In another embodiment, the attenuated strains is Lm dal(−)dat(−)ΔactA (LmddA). LmddA is based on a Listeria immunotherapy vector which is attenuated due to the deletion of virulence gene actA and retains the plasmid for a desired heterologous antigen or truncated LLO expression in vivo and in vitro by complementation of dal gene.

In another embodiment the attenuated strain is LmddA. In another embodiment, the attenuated strain is LmΔactA. In another embodiment, the attenuated strain is LmΔPrfA. In another embodiment, the attenuated strain is LmΔPrfA*. In another embodiment, the attenuated strain is LmΔPlcB. In another embodiment, the attenuated strain is LmΔPlcA. In another embodiment, the strain is the double mutant or triple mutant of any of the above-mentioned strains. In another embodiment, this strain exerts a strong adjuvant effect which is an inherent property of Listeria-based immunotherapies. In another embodiment, this strain is constructed from the EGD Listeria backbone. In another embodiment, the strain used in the invention is a Listeria strain that expresses a non-hemolytic LLO.

In another embodiment, the Listeria strain is an auxotrophic mutant. In another embodiment, the Listeria strain is deficient in a gene encoding a vitamin synthesis gene. In another embodiment, the Listeria strain is deficient in a gene encoding pantothenic acid synthase.

In one embodiment, the generation of AA strains of Listeria deficient in D-alanine, for example, may be accomplished in a number of ways that are well known to those of skill in the art, including deletion mutagenesis, insertion mutagenesis, and mutagenesis which results in the generation of frameshift mutations, mutations which cause premature termination of a protein, or mutation of regulatory sequences which affect gene expression. In another embodiment, mutagenesis can be accomplished using recombinant DNA techniques or using traditional mutagenesis technology using mutagenic chemicals or radiation and subsequent selection of mutants. In another embodiment, deletion mutants are preferred because of the accompanying low probability of reversion of the auxotrophic phenotype. In another embodiment, mutants of D-alanine which are generated according to the protocols presented herein may be tested for the ability to grow in the absence of D-alanine in a simple laboratory culture assay. In another embodiment, those mutants which are unable to grow in the absence of this compound are selected for further study.

In another embodiment, in addition to the aforementioned D-alanine associated genes, other genes involved in synthesis of a metabolic enzyme, as provided herein, may be used as targets for mutagenesis of Listeria.

In another embodiment, the metabolic enzyme complements an endogenous metabolic gene that is lacking in the remainder of the chromosome of the recombinant bacterial strain. In one embodiment, the endogenous metabolic gene is mutated in the chromosome. In another embodiment, the endogenous metabolic gene is deleted from the chromosome. In another embodiment, the metabolic enzyme is an amino acid metabolism enzyme. In another embodiment, the metabolic enzyme catalyzes a formation of an amino acid used for a cell wall synthesis in the recombinant Listeria strain. In another embodiment, the metabolic enzyme is an alanine racemase enzyme. In another embodiment, the metabolic enzyme is a D-amino acid transferase enzyme. Each possibility represents a separate embodiment of the methods and compositions as provided herein.

In one embodiment, the auxotrophic Listeria strain comprises an episomal expression vector comprising a metabolic enzyme that complements the auxotrophy of the auxotrophic Listeria strain. In another embodiment, the construct is contained in the Listeria strain in an episomal fashion. In another embodiment, the foreign antigen is expressed from a plasmid vector harbored by the recombinant Listeria strain. In another embodiment, the episomal expression plasmid vector lacks an antibiotic resistance marker. In one embodiment, an antigen of the methods and compositions as provided herein is fused to an polypeptide comprising a PEST sequence.

In another embodiment, the Listeria strain is deficient in an amino acid (AA) metabolism enzyme. In another embodiment, the Listeria strain is deficient in a D-glutamic acid synthase gene. In another embodiment, the Listeria strain is deficient in the dat gene. In another embodiment, the Listeria strain is deficient in the dal gene. In another embodiment, the Listeria strain is deficient in the dga gene. In another embodiment, the Listeria strain is deficient in a gene involved in the synthesis of diaminopimelic acid. CysK. In another embodiment, the gene is vitamin-B12 independent methionine synthase. In another embodiment, the gene is trpA. In another embodiment, the gene is trpB. In another embodiment, the gene is trpE. In another embodiment, the gene is asnB. In another embodiment, the gene is gltD. In another embodiment, the gene is gltB. In another embodiment, the gene is leuA. In another embodiment, the gene is argG. In another embodiment, the gene is thrC. In another embodiment, the Listeria strain is deficient in one or more of the genes described hereinabove.

In another embodiment, the Listeria strain is deficient in a synthase gene. In another embodiment, the gene is an AA synthesis gene. In another embodiment, the gene is folP. In another embodiment, the gene is dihydrouridine synthase family protein. In another embodiment, the gene is ispD. In another embodiment, the gene is ispF. In another embodiment, the gene is phosphoenolpyruvate synthase. In another embodiment, the gene is hisF. In another embodiment, the gene is hisH. In another embodiment, the gene is fliI. In another embodiment, the gene is ribosomal large subunit pseudouridine synthase. In another embodiment, the gene is ispD. In another embodiment, the gene is bifunctional GMP synthase/glutamine amidotransferase protein. In another embodiment, the gene is cobS. In another embodiment, the gene is cobB. In another embodiment, the gene is cbiD. In another embodiment, the gene is uroporphyrin-III C-methyltransferase/uroporphyrinogen-III synthase. In another embodiment, the gene is cobQ. In another embodiment, the gene is uppS. In another embodiment, the gene is truB. In another embodiment, the gene is dxs. In another embodiment, the gene is mvaS. In another embodiment, the gene is dapA. In another embodiment, the gene is ispG. In another embodiment, the gene is folC. In another embodiment, the gene is citrate synthase. In another embodiment, the gene is argJ. In another embodiment, the gene is 3-deoxy-7-phosphoheptulonate synthase. In another embodiment, the gene is indole-3-glycerol-phosphate synthase. In another embodiment, the gene is anthranilate synthase/glutamine amidotransferase component. In another embodiment, the gene is menB. In another embodiment, the gene is menaquinone-specific isochorismate synthase. In another embodiment, the gene is phosphoribosylformylglycinamidine synthase I or II. In another embodiment, the gene is phosphoribosylaminoimidazole-succinocarboxamide synthase. In another embodiment, the gene is carB. In another embodiment, the gene is carA. In another embodiment, the gene is thyA. In another embodiment, the gene is mgsA. In another embodiment, the gene is aroB. In another embodiment, the gene is hepB. In another embodiment, the gene is rluB. In another embodiment, the gene is ilvB. In another embodiment, the gene is ilvN. In another embodiment, the gene is alsS. In another embodiment, the gene is fabF. In another embodiment, the gene is fabH. In another embodiment, the gene is pseudouridine synthase. In another embodiment, the gene is pyrG. In another embodiment, the gene is truA. In another embodiment, the gene is pabB. In another embodiment, the gene is an atp synthase gene (e.g. atpC, atpD-2, aptG, atpA-2, etc).

In another embodiment, the gene is phoP. In another embodiment, the gene is aroA. In another embodiment, the gene is aroC. In another embodiment, the gene is aroD. In another embodiment, the gene is plcB.

In another embodiment, the Listeria strain is deficient in a peptide transporter. In another embodiment, the gene is ABC transporter/ATP-binding/permease protein. In another embodiment, the gene is oligopeptide ABC transporter/oligopeptide-binding protein. In another embodiment, the gene is oligopeptide ABC transporter/permease protein. In another embodiment, the gene is zinc ABC transporter/zinc-binding protein. In another embodiment, the gene is sugar ABC transporter. In another embodiment, the gene is phosphate transporter. In another embodiment, the gene is ZIP zinc transporter. In another embodiment, the gene is drug resistance transporter of the EmrB/QacA family. In another embodiment, the gene is sulfate transporter. In another embodiment, the gene is proton-dependent oligopeptide transporter. In another embodiment, the gene is magnesium transporter. In another embodiment, the gene is formate/nitrite transporter. In another embodiment, the gene is spermidine/putrescine ABC transporter. In another embodiment, the gene is Na/Pi-cotransporter. In another embodiment, the gene is sugar phosphate transporter. In another embodiment, the gene is glutamine ABC transporter. In another embodiment, the gene is major facilitator family transporter. In another embodiment, the gene is glycine betaine/L-proline ABC transporter. In another embodiment, the gene is molybdenum ABC transporter. In another embodiment, the gene is techoic acid ABC transporter. In another embodiment, the gene is cobalt ABC transporter. In another embodiment, the gene is ammonium transporter. In another embodiment, the gene is amino acid ABC transporter. In another embodiment, the gene is cell division ABC transporter. In another embodiment, the gene is manganese ABC transporter. In another embodiment, the gene is iron compound ABC transporter. In another embodiment, the gene is maltose/maltodextrin ABC transporter. In another embodiment, the gene is drug resistance transporter of the Bcr/CflA family. In another embodiment, the gene is a subunit of one of the above proteins.

In one embodiment, provided herein is a nucleic acid molecule that is used to transform the Listeria in order to arrive at a recombinant Listeria. In another embodiment, the nucleic acid provided herein used to transform Listeria lacks a virulence gene. In another embodiment, the nucleic acid molecule is integrated into the Listeria genome and carries a non-functional virulence gene. In another embodiment, the virulence gene is mutated in the recombinant Listeria. In yet another embodiment, the nucleic acid molecule is used to inactivate the endogenous gene present in the Listeria genome. In yet another embodiment, the virulence gene is an actA gene, an inlA gene, and inlB gene, an inlC gene, inlJ gene, a plbC gene, a bsh gene, or a prfA gene. It is to be understood by a skilled artisan, that the virulence gene can be any gene known in the art to be associated with virulence in the recombinant Listeria.

In yet another embodiment the Listeria strain is an inlA mutant, an inlB mutant, an inlC mutant, an inlJ mutant, prfA mutant, actA mutant, a dal/dat mutant, a prfA mutant, a plcB deletion mutant, or a double mutant lacking both plcA and plcB or actA and inlB. In another embodiment, the Listeria comprise a deletion or mutation of these genes individually or in combination. In another embodiment, the Listeria provided herein lack each one of genes. In another embodiment, the Listeria provided herein lack at least one and up to ten of any gene provided herein, including the actA, prfA, and dal/dat genes. In another embodiment, the prfA mutant is a D133V prfA mutant.

In one embodiment, the live attenuated Listeria is a recombinant Listeria. In another embodiment, the recombinant Listeria comprises a mutation or a deletion of a genomic internalin C (inlC) gene. In another embodiment, the recombinant Listeria comprises a mutation or a deletion of a genomic actA gene and a genomic internalin C gene. In one embodiment, translocation of Listeria to adjacent cells is inhibited by the deletion of the actA gene and/or the inlC gene, which are involved in the process, thereby resulting in unexpectedly high levels of attenuation with increased immunogenicity and utility as a immunotherapy backbone.

In one embodiment, the metabolic gene, the virulence gene, etc. is lacking in a chromosome of the Listeria strain. In another embodiment, the metabolic gene, virulence gene, etc. is lacking in the chromosome and in any episomal genetic element of the Listeria strain. In another embodiment, the metabolic gene, virulence gene, etc. is lacking in the genome of the virulence strain. In one embodiment, the virulence gene is mutated in the chromosome. In another embodiment, the virulence gene is deleted from the chromosome.

In one embodiment, the recombinant Listeria strain provided herein is attenuated. In another embodiment, the recombinant Listeria lacks the actA virulence gene. In another embodiment, the recombinant Listeria lacks the prfA virulence gene. In another embodiment, the recombinant Listeria lacks the inlB gene. In another embodiment, the recombinant Listeria lacks both, the actA and inlB genes. In another embodiment, the recombinant Listeria strain provided herein comprise an inactivating mutation of the endogenous actA gene. In another embodiment, the recombinant Listeria strain provided herein comprise an inactivating mutation of the endogenous inlB gene. In another embodiment, the recombinant Listeria strain provided herein comprise an inactivating mutation of the endogenous inlC gene. In another embodiment, the recombinant Listeria strain provided herein comprise an inactivating mutation of the endogenous actA and inlB genes. In another embodiment, the recombinant Listeria strain provided herein comprise an inactivating mutation of the endogenous actA and inlC genes. In another embodiment, the recombinant Listeria strain provided herein comprise an inactivating mutation of the endogenous actA, inlB, and inlC genes. In another embodiment, the recombinant Listeria strain provided herein comprise an inactivating mutation of the endogenous actA, inlB, and inlC genes. In another embodiment, the recombinant Listeria strain provided herein comprise an inactivating mutation of the endogenous actA, inlB, and inlC genes. In another embodiment, the recombinant Listeria strain provided herein comprise an inactivating mutation in any single gene or combination of the following genes: actA, dal, dat, inlB, inlC, prfA, plcA, plcB.

It will be appreciated by the skilled artisan that the term “mutation” and grammatical equivalents thereof, include any type of mutation or modification to the sequence (nucleic acid or amino acid sequence), and includes a deletion mutation, a truncation, an inactivation, a disruption, or a translocation. These types of mutations are readily known in the art.

In one embodiment, in order to select for an auxotrophic bacteria comprising a plasmid encoding a metabolic enzyme or a complementing gene provided herein, transformed auxotrophic bacteria are grown on a media that will select for expression of the amino acid metabolism gene or the complementing gene. In another embodiment, a bacteria auxotrophic for D-glutamic acid synthesis is transformed with a plasmid comprising a gene for D-glutamic acid synthesis, and the auxotrophic bacteria will grow in the absence of D-glutamic acid, whereas auxotrophic bacteria that have not been transformed with the plasmid, or are not expressing the plasmid encoding a protein for D-glutamic acid synthesis, will not grow. In another embodiment, a bacterium auxotrophic for D-alanine synthesis will grow in the absence of D-alanine when transformed and expressing the plasmid of the present invention if the plasmid comprises an isolated nucleic acid encoding an amino acid metabolism enzyme for D-alanine synthesis. Such methods for making appropriate media comprising or lacking necessary growth factors, supplements, amino acids, vitamins, antibiotics, and the like are well known in the art, and are available commercially (Becton-Dickinson, Franklin Lakes, N.J.). Each method represents a separate embodiment of the present invention.

In another embodiment, once the auxotrophic bacteria comprising the plasmid of the present invention have been selected on appropriate media, the bacteria are propagated in the presence of a selective pressure. Such propagation comprises growing the bacteria in media without the auxotrophic factor. The presence of the plasmid expressing an amino acid metabolism enzyme in the auxotrophic bacteria ensures that the plasmid will replicate along with the bacteria, thus continually selecting for bacteria harboring the plasmid. The skilled artisan, when equipped with the present disclosure and methods herein will be readily able to scale-up the production of the Listeria immunotherapy vector by adjusting the volume of the media in which the auxotrophic bacteria comprising the plasmid are growing.

The skilled artisan will appreciate that, in another embodiment, other auxotroph strains and complementation systems are adopted for the use with this invention.

In one embodiment, the N-terminal LLO protein fragment and heterologous antigen are fused directly to one another. In another embodiment, the genes encoding the N-terminal LLO protein fragment and heterologous antigen are fused directly to one another. In another embodiment, the N-terminal LLO protein fragment and heterologous antigen are operably attached via a linker peptide. In another embodiment, the N-terminal LLO protein fragment and heterologous antigen are attached via a heterologous peptide. In another embodiment, the N-terminal LLO protein fragment is N-terminal to the heterologous antigen. In another embodiment, the N-terminal LLO protein fragment is expressed and used alone, i.e., in unfused form. In another embodiment, an N-terminal LLO protein fragment is the N-terminal-most portion of the fusion protein. In another embodiment, a truncated LLO is truncated at the C-terminal to arrive at an N-terminal LLO. In another embodiment, a truncated LLO is a non-hemolytic LLO.

In one embodiment, the N-terminal ActA protein fragment and heterologous antigen are fused directly to one another. In another embodiment, the genes encoding the N-terminal ActA protein fragment and heterologous antigen are fused directly to one another. In another embodiment, the N-terminal ActA protein fragment and heterologous antigen are operably attached via a linker peptide. In another embodiment, the N-terminal ActA protein fragment and heterologous antigen are attached via a heterologous peptide. In another embodiment, the N-terminal ActA protein fragment is N-terminal to the heterologous antigen. In another embodiment, the N-terminal ActA protein fragment is expressed and used alone, i.e., in unfused form. In another embodiment, the N-terminal ActA protein fragment is the N-terminal-most portion of the fusion protein. In another embodiment, a truncated ActA is truncated at the C-terminal to arrive at an N-terminal ActA.

In one embodiment, the recombinant Listeria strain provided herein expresses the recombinant polypeptide. In another embodiment, the recombinant Listeria strain comprises a plasmid that encodes the recombinant polypeptide. In another embodiment, a recombinant nucleic acid provided herein is in a plasmid in the recombinant Listeria strain provided herein. In another embodiment, the plasmid is an episomal plasmid that does not integrate into the recombinant Listeria strain's chromosome. In another embodiment, the plasmid is an integrative plasmid that integrates into the Listeria strain's chromosome. In another embodiment, the plasmid is a multicopy plasmid.

In one embodiment, the heterologous antigen is a tumor-associated antigen. In one embodiment, the recombinant Listeria strain of the compositions and methods as provided herein express a heterologous antigenic polypeptide that is expressed by a tumor cell. In one embodiment, a tumor-associated antigen is a prostate specific antigen (PSA). In another embodiment, a tumor-associated antigen is a human papilloma virus (HPV) antigen. In yet another embodiment, a tumor-associated antigen is a Her2/neu chimeric antigen as described in US Patent Pub. No. US2011/014279, which is incorporated by reference herein in its entirety. In still another embodiment, a tumor-associated antigen is an angiogenic antigen.

In one embodiment, the peptide provided herein is an antigenic peptide. In another embodiment, the peptide provided herein is derived from a tumor antigen. In another embodiment, the peptide provided herein is derived from an infectious disease antigen. In another embodiment, the peptide provided herein is derived from a self-antigen. In another embodiment, the peptide provided herein is derived from an angiogenic antigen.

In one embodiment, the antigen from which the peptide provided herein is derived from is derived from a fungal pathogen, bacteria, parasite, helminth, or viruses. In other embodiments, the antigen from which the peptide derived herein is selected from tetanus toxoid, hemagglutinin molecules from influenza virus, diphtheria toxoid, HIV gp120, HIV gag protein, IgA protease, insulin peptide B, Spongospora subterranea antigen, vibriose antigens, Salmonella antigens, pneumococcus antigens, respiratory syncytial virus antigens, Haemophilus influenza outer membrane proteins, Helicobacter pylori urease, Neisseria meningitidis pilins, N. gonorrhoeae pilins, the melanoma-associated antigens (TRP-2, MAGE-1, MAGE-3, gp-100, tyrosinase, MART-1, HSP-70, beta-HCG), human papilloma virus antigens E1 and E2 from type HPV-16, -18, -31, -33, -35 or -45 human papilloma viruses, the tumor antigens CEA, the ras protein, mutated or otherwise, the p53 protein, mutated or otherwise, Muc1, mesothelin, EGFRVIII or pSA.

In other embodiments, the peptide is derived from an antigen that is associated with one of the following diseases; cholera, diphtheria, Haemophilus, hepatitis A, hepatitis B, influenza, measles, meningitis, mumps, pertussis, small pox, pneumococcal pneumonia, polio, rabies, rubella, tetanus, tuberculosis, typhoid, Varicella-zoster, whooping cough, yellow fever, the immunogens and antigens from Addison's disease, allergies, anaphylaxis, Bruton's syndrome, cancer, including solid and blood borne tumors, eczema, Hashimoto's thyroiditis, polymyositis, dermatomyositis, type 1 diabetes mellitus, acquired immune deficiency syndrome, transplant rejection, such as kidney, heart, pancreas, lung, bone, and liver transplants, Graves' disease, polyendocrine autoimmune disease, hepatitis, microscopic polyarteritis, polyarteritis nodosa, pemphigus, primary biliary cirrhosis, pernicious anemia, coeliac disease, antibody-mediated nephritis, glomerulonephritis, rheumatic diseases, systemic lupus erthematosus, rheumatoid arthritis, seronegative spondylarthritides, rhinitis, sjogren's syndrome, systemic sclerosis, sclerosing cholangitis, Wegener's granulomatosis, dermatitis herpetiformis, psoriasis, vitiligo, multiple sclerosis, encephalomyelitis, Guillain-Barre syndrome, myasthenia gravis, Lambert-Eaton syndrome, sclera, episclera, uveitis, chronic mucocutaneous candidiasis, urticaria, transient hypogammaglobulinemia of infancy, myeloma, X-linked hyper IgM syndrome, Wiskott-Aldrich syndrome, ataxia telangiectasia, autoimmune hemolytic anemia, autoimmune thrombocytopenia, autoimmune neutropenia, Waldenstrom's macroglobulinemia, amyloidosis, chronic lymphocytic leukemia, non-Hodgkin's lymphoma, malarial circumsporozite protein, microbial antigens, viral antigens, autoantigens, and lesteriosis.

In another embodiment, the antigen from which the peptide provided herein is derived is a tumor-associated antigen, which in one embodiment, is one of the following tumor antigens: a MAGE (Melanoma-Associated Antigen E) protein, e.g. MAGE 1, MAGE 2, MAGE 3, MAGE 4, a tyrosinase; a mutant ras protein; a mutant p53 protein; p97 melanoma antigen, a ras peptide or p53 peptide associated with advanced cancers; the HPV 16/18 antigens associated with cervical cancers, KLH antigen associated with breast carcinoma, CEA (carcinoembryonic antigen) associated with colorectal cancer, gp100, a MART1 antigen associated with melanoma, or the PSA antigen associated with prostate cancer. In another embodiment, the antigen for the compositions and methods as provided herein are melanoma-associated antigens, which in one embodiment are TRP-2, MAGE-1, MAGE-3, gp-100, tyrosinase, HSP-70, beta-HCG, or a combination thereof. Other tumor-associated antigens known in the art are also contemplated in the present invention.

In one embodiment, the peptide is derived from a chimeric Her2 antigen described in U.S. patent application Ser. No. 12/945,386, which is hereby incorporated by reference herein in its entirety.

In another embodiment, the peptide is derived from an antigen selected from a HPV-E7 (from either an HPV16 or HPV18 strain), a HPV-E6 (from either an HPV16 or HPV18 strain), Her-2/neu, NY-ESO-1, telomerase (TERT, SCCE, CEA, LMP-1, p53, carboxic anhydrase IX (CALX), PSMA, a prostate stem cell antigen (PSCA), a HMW-MAA, WT-1, HIV-1 Gag, Proteinase 3, Tyrosinase related protein 2, PSA (prostate-specific antigen), EGFR-III, survivin, baculoviral inhibitor of apoptosis repeat-containing 5 (BIRC5), LMP-1, p53, PSMA, PSCA, Muc1, PSA (prostate-specific antigen), or a combination thereof.

In one embodiment, a polypeptide expressed by the Listeria of the present invention may be a neuropeptide growth factor antagonist, which in one embodiment is [D-Arg1, D-Phe5, D-Trp7,9, Leu11] substance P, [Arg6, D-Trp7,9, NmePhe8]substance P(6-11). These and related embodiments are understood by one of skill in the art.

In one embodiment, the recombinant Listeria strain as provided herein comprises a nucleic acid molecule encoding a tumor associated antigen, wherein the antigen comprises an HPV-E7 protein. In one embodiment, the recombinant Listeria strain as provided herein comprises a nucleic acid molecule encoding HPV-E7 protein.

In one embodiment, either a whole E7 protein or a fragment thereof is fused to a LLO protein or truncation or peptide thereof, an ActA protein or truncation or peptide thereof, or a PEST-like sequence-containing peptide to generate a recombinant polypeptide or peptide of the composition and methods of the present invention. The E7 protein that is utilized (either whole or as the source of the fragments) has, in another embodiment, the sequence MHGDTPTLHEYMLDLQPETTDLYCYEQLNDSSEEEDEIDGPAGQAEPDRAHYNIVTF CCKCDSTLRLCVQSTHVDIRTLEDLLMGTLGIVCPICSQKP (SEQ ID No: 20). In another embodiment, the E7 protein is a homologue of SEQ ID No: 20. In another embodiment, the E7 protein is a variant of SEQ ID No: 20. In another embodiment, the E7 protein is an isomer of SEQ ID No: 20. In another embodiment, the E7 protein is a fragment of SEQ ID No: 20. In another embodiment, the E7 protein is a fragment of a homologue of SEQ ID No: 20. In another embodiment, the E7 protein is a fragment of a variant of SEQ ID No: 20. In another embodiment, the E7 protein is a fragment of an isomer of SEQ ID No: 20.

In another embodiment, the sequence of the E7 protein is: MHGPKATLQDIVLHLEPQNEIPVDLLCHEQLSDSEEENDEIDGVNHQHLPARRAEPQ RHTMLCMCCKCEARIELVVESSADDLRAFQQLFLNTLSFVCPWCASQQ (SEQ ID No: 21). In another embodiment, the E6 protein is a homologue of SEQ ID No: 21. In another embodiment, the E6 protein is a variant of SEQ ID No: 21. In another embodiment, the E6 protein is an isomer of SEQ ID No: 21. In another embodiment, the E6 protein is a fragment of SEQ ID No: 21. In another embodiment, the E6 protein is a fragment of a homologue of SEQ ID No: 21. In another embodiment, the E6 protein is a fragment of a variant of SEQ ID No: 21. In another embodiment, the E6 protein is a fragment of an isomer of SEQ ID No: 21.

In another embodiment, the E7 protein has a sequence set forth in one of the following GenBank entries: M24215, NC_004500, V01116, X62843, or M14119. In another embodiment, the E7 protein is a homologue of a sequence from one of the above GenBank entries. In another embodiment, the E7 protein is a variant of a sequence from one of the above GenBank entries. In another embodiment, the E7 protein is an isomer of a sequence from one of the above GenBank entries. In another embodiment, the E7 protein is a fragment of a sequence from one of the above GenBank entries. In another embodiment, the E7 protein is a fragment of a homologue of a sequence from one of the above GenBank entries. In another embodiment, the E7 protein is a fragment of a variant of a sequence from one of the above GenBank entries. In another embodiment, the E7 protein is a fragment of an isomer of a sequence from one of the above GenBank entries.

In one embodiment the HPV antigen is an HPV 16. In another embodiment, the HPV is an HPV-18. In another embodiment, the HPV is selected from HPV-16 and HPV-18. In another embodiment, the HPV is an HPV-31. In another embodiment, the HPV is an HPV-35. In another embodiment, the HPV is an HPV-39. In another embodiment, the HPV is an HPV-45. In another embodiment, the HPV is an HPV-51. In another embodiment, the HPV is an HPV-52. In another embodiment, the HPV is an HPV-58. In another embodiment, the HPV is a high-risk HPV type. In another embodiment, the HPV is a mucosal HPV type.

In one embodiment, the HPV E6 is from HPV-16. In another embodiment, the HPV E7 is from HPV-16. In another embodiment, the HPV-E6 is from HPV-18. In another embodiment, the HPV-E7 is from HPV-18. In another embodiment, an HPV E6 antigen is utilized instead of or in addition to an E7 antigen in a composition or method of the present invention for treating or ameliorating an HPV-mediated disease, disorder, or symptom. In another embodiment, an HPV-16 E6 and E7 is utilized instead of or in combination with an HPV-18 E6 and E7. In such an embodiment, the recombinant Listeria may express the HPV-16 E6 and E7 from the chromosome and the HPV-18 E6 and E7 from a plasmid, or vice versa. In another embodiment, the HPV-16 E6 and E7 antigens and the HPV-18 E6 and E7 antigens are expressed from a plasmid present in a recombinant Listeria provided herein. In another embodiment, the HPV-16 E6 and E7 antigens and the HPV-18 E6 and E7 antigens are expressed from the chromosome of a recombinant Listeria provided herein. In another embodiment, the HPV-16 E6 and E7 antigens and the HPV-18 E6 and E7 antigens are expressed in any combination of the above embodiments, including where each E6 and E7 antigen from each HPV strain is expressed from either the plasmid or the chromosome.

In one embodiment, the recombinant Listeria strain as provided herein comprises a nucleic acid molecule encoding a tumor associated antigen, wherein the tumor associated antigen comprises a Her-2/neu peptide. In one embodiment, a tumor associated antigen comprises a Her-2/neu antigen. In one embodiment the Her-2/neu peptide comprises a chimeric Her-2/neu antigen (cHer-2).

In one embodiment, the attenuated auxotrophic Listeria immunotherapy strain is based on a Listeria immunotherapy vector which is attenuated due to the deletion of virulence gene actA and retains the plasmid for Her2/neu expression in vivo and in vitro by complementation of dal gene. In one embodiment, the Listeria strain expresses and secretes a chimeric Her2/neu protein fused to the first 441 amino acids of listeriolysin O (LLO). In another embodiment, the Listeria is a dal/dat/actA Listeria having a mutation in the dal, dat and actA endogenous genes. In another embodiment, the mutation is a deletion, a truncation or an inactivation of the mutated genes. In another embodiment, Listeria strain exerts strong and antigen specific anti-tumor responses with ability to break tolerance toward HER2/neu in transgenic animals. In another embodiment, the dal/dat/actA strain is highly attenuated and has a better safety profile than previous Listeria immunotherapy generation, as it is more rapidly cleared from the spleens of the immunized mice. In another embodiment, the Listeria strain results in a longer delay of tumor onset in transgenic animals than Lm-LLO-ChHer2, the antibiotic resistant and more virulent version of this immunotherapy see U.S. Ser. No. 12/945,386; US Publication No. 2011/0142791, which is incorporated by reference herein in its entirety). In another embodiment, the Listeria strain causes a significant decrease in intra-tumoral T regulatory cells (Tregs). In another embodiment, the lower frequency of Tregs in tumors treated with LmddA immunotherapies result in an increased intratumoral CD8/Tregs ratio, suggesting that a more favorable tumor microenvironment can be obtained after immunization with LmddA immunotherapies. In one embodiment, the present invention provides a recombinant polypeptide comprising an N-terminal fragment of an LLO protein fused to a Her-2 chimeric protein or fused to a fragment thereof. In one embodiment, the present invention provides a recombinant polypeptide consisting of an N-terminal fragment of an LLO protein fused to a Her-2 chimeric protein or fused to a fragment thereof. In the embodiment, the heterologous antigen is a Her-2 chimeric protein or fragment thereof.

In another embodiment, the Her-2 chimeric protein of the methods and compositions of the present invention is a human Her-2 chimeric protein. In another embodiment, the Her-2 protein is a mouse Her-2 chimeric protein. In another embodiment, the Her-2 protein is a rat Her-2 chimeric protein. In another embodiment, the Her-2 protein is a primate Her-2 chimeric protein. In another embodiment, the Her-2 protein is a Her-2 chimeric protein of human or any other animal species or combinations thereof known in the art.

In another embodiment, a Her-2 protein is a protein referred to as “HER-2/neu,” “Erbb2,” “v-erb-b2,” “c-erb-b2,” “neu,” or “cNeu.”

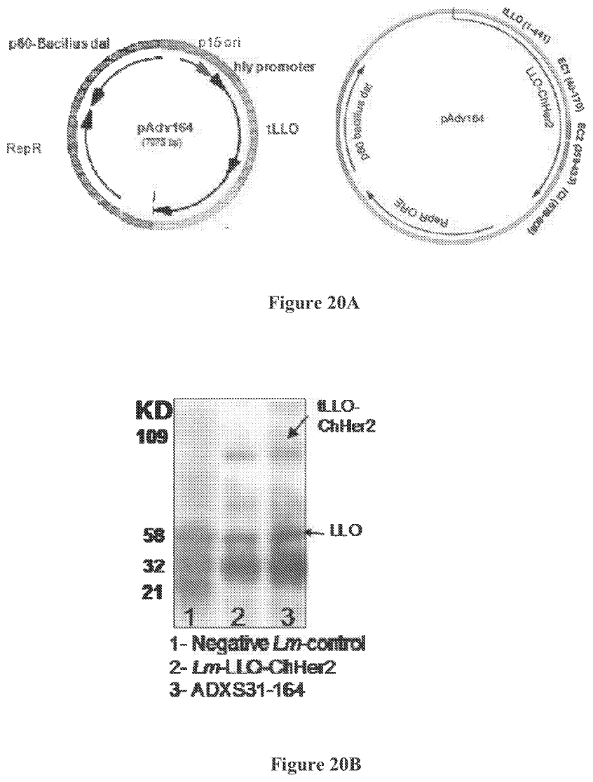

In one embodiment, the Her2-neu chimeric protein, harbors two of the extracellular and one intracellular fragments of Her2/neu antigen showing clusters of MHC-class I epitopes of the oncogene, where, in another embodiment, the chimeric protein harbors 3 H2Dq and at least 17 of the mapped human MHC-class I epitopes of the Her2/neu antigen (fragments EC1, EC2, and IC1) (FIG. 20A. In another embodiment, the chimeric protein harbors at least 13 of the mapped human MHC-class I epitopes (fragments EC2 and IC1). In another embodiment, the chimeric protein harbors at least 14 of the mapped human MHC-class I epitopes (fragments EC1 and IC1). In another embodiment, the chimeric protein harbors at least 9 of the mapped human MHC-class I epitopes (fragments EC1 and IC2). In another embodiment, the Her2-neu chimeric protein is fused to a non-hemolytic listeriolysin O (LLO). In another embodiment, the Her2-neu chimeric protein is fused to the first 441 amino acids of the Listeria-monocytogenes listeriolysin O (LLO) protein and expressed and secreted by the Listeria monocytogenes attenuated auxotrophic strain LmddA. In another embodiment, the expression and secretion of the fusion protein tLLO-ChHer2 from the attenuated auxotrophic strain provided herein that expresses a chimeric Her2/neu antigen/LLO fusion protein is comparable to that of the Lm-LLO-ChHer2 in TCA precipitated cell culture supernatants after 8 hours of in vitro growth (FIG. 20B).

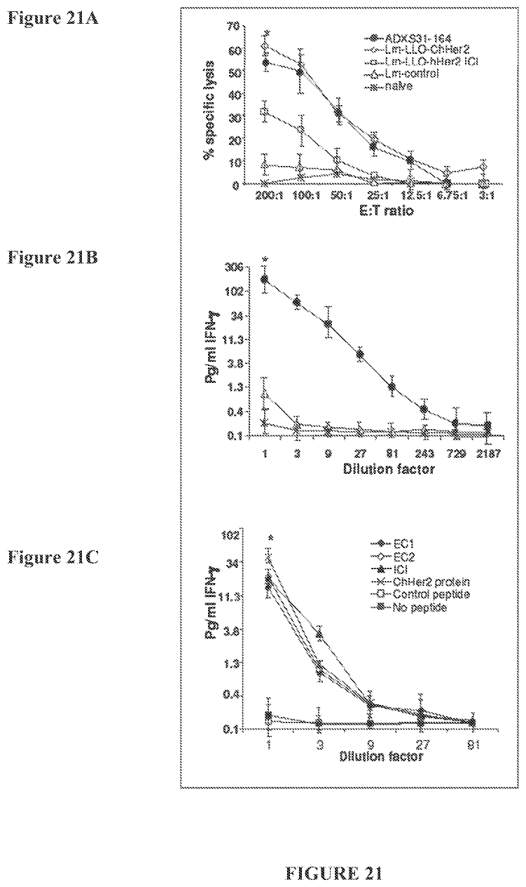

In one embodiment, no CTL activity is detected in naïve animals or mice injected with an irrelevant Listeria immunotherapy (FIG. 21A). While in another embodiment, the attenuated auxotrophic strain provided herein is able to stimulate the secretion of IFN-γ by the splenocytes from wild type FVB/N mice (FIGS. 21B and 21C).

In another embodiment, the Her-2 chimeric protein is encoded by the following nucleic acid sequence set forth in SEQ ID NO:22:

(SEQ ID NO: 22)
gagacccacctggacatgctccgccacctctaccagggctgccaggtggt
gcagggaaacctggaactcacctacctgcccaccaatgccagcctgtcct
tcctgcaggatatccaggaggtgcagggctacgtgctcatcgctcacaac
caagtgaggcaggtcccactgcagaggctgcggattgtgcgaggcaccca
gctctttgaggacaactatgccctggccgtgctagacaatggagacccgc
tgaacaataccacccctgtcacaggggcctccccaggaggcctgcgggag
ctgcagcttcgaagcctcacagagatcttgaaaggaggggtcttgatcca
gcggaacccccagctctgctaccaggacacgattttgtggaagaatatcc
aggagtttgctggctgcaagaagatctttgggagcctggcatttctgccg
gagagctttgatggggacccagcctccaacactgccccgctccagccaga
gcagctccaagtgtttgagactctggaagagatcacaggttacctataca
tctcagcatggccggacagcctgcctgacctcagcgtcttccagaacctg
caagtaatccggggacgaattctgcacaatggcgcctactcgctgaccct
gcaagggctgggcatcagctggctggggctgcgctcactgagggaactgg
gcagtggactggccctcatccaccataacacccacctctgcttcgtgcac
acggtgccctgggaccagctctttcggaacccgcaccaagctctgctcca
cactgccaaccggccagaggacgagtgtgtgggcgagggcctggcctgcc
accagctgtgcgcccgagggcagcagaagatccggaagtacacgatgcgg
agactgctgcaggaaacggagctggtggagccgctgacacctagcggagc
gatgcccaaccaggcgcagatgcggatcctgaaagagacggagctgagga
aggtgaaggtgcttggatctggcgcttttggcacagtctacaagggcatc
tggatccctgatggggagaatgtgaaaattccagtggccatcaaagtgtt
gagggaaaacacatcccccaaagccaacaaagaaatcttagacgaagcat
acgtgatggctggtgtgggctccccatatgtctcccgccttctgggcatc
tgcctgacatccacggtgcagctggtgacacagcttatgccctatggctg
cctcttagactaa.

In another embodiment, the Her-2 chimeric protein has the sequence:

(SEQ ID NO: 23)
ETHLDMLRHLYQGCQVVQGNLELTYLPTNASLSFLQDIQEV
QGYVLIAHNQVRQVPLQRLRIVRGTQLFEDNYALAVLDNGD
PLNNTTPVTGASPGGLRELQLRSLTEILKGGVLIQRNPQLCY
QDTILWKNIQEFAGCKKIFGSLAFLPESFDGDPASNTAPLQP
EQLQVFETLEEITGYLYISAWPDSLPDLSVFQNLQVIRGRILH
NGAYSLTLQGLGISWLGLRSLRELGSGLALIHHNTHLCFVHT
VPWDQLFRNPHQALLHTANRPEDECVGEGLACHQLCARGQ
QKIRKYTMRRLLQETELVEPLTPSGAMPNQAQMRILKETEL
RKVKVLGSGAFGTVYKGIWIPDGENVKIPVAIKVLRENTSPK
ANKEILDEAYVMAGVGSPYVSRLLGICLTSTVQLVTQLMPY
GCLLD.

In one embodiment, the Her2 chimeric protein or fragment thereof of the methods and compositions provided herein does not include a signal sequence thereof. In another embodiment, omission of the signal sequence enables the Her2 fragment to be successfully expressed in Listeria, due the high hydrophobicity of the signal sequence.

In another embodiment, the fragment of a Her2 chimeric protein of methods and compositions of the present invention does not include a transmembrane domain (TM) thereof. In one embodiment, omission of the TM enables the Her-2 fragment to be successfully expressed in Listeria, due the high hydrophobicity of the TM.

Point mutations or amino-acid deletions in the oncogenic protein Her2/neu, have been reported to mediate treatment of resistant tumor cells, when these tumors have been targeted by small fragment Listeria-based immunotherapies or trastuzumab (a monoclonal antibody against an epitope located at the extracellular domain of the Her2/neu antigen). Described herein is a chimeric Her2/neu based composition which harbors two of the extracellular and one intracellular fragments of Her2/neu antigen showing clusters of MHC-class I epitopes of the oncogene. This chimeric protein, which harbors 3 H2Dq and at least 17 of the mapped human MHC-class I epitopes of the Her2/neu antigen was fused to the first 441 amino acids of the Listeria-monocytogenes listeriolysin 0 protein and expressed and secreted by the Listeria monocytogenes attenuated strain LmddA.

In another embodiment, the tumor-associated antigen is an angiogenic antigen. In another embodiment, the angiogenic antigen is expressed on both activated pericytes and pericytes in tumor angiogeneic vasculature, which in another embodiment, is associated with neovascularization in vivo. In another embodiment, the angiogenic antigen is HMW-MAA. In another embodiment, the angiogenic antigen is one known in the art and are provided in WO2010/102140, which is incorporated by reference herein.

Protein and/or peptide homology for any amino acid sequence listed herein is determined, in one embodiment, by methods well described in the art, including immunoblot analysis, or via computer algorithm analysis of amino acid sequences, utilizing any of a number of software packages available, via established methods. Some of these packages may include the FASTA, BLAST, MPsrch or Scanps packages, and may employ the use of the Smith and Waterman algorithms, and/or global/local or BLOCKS alignments for analysis, for example.

In one embodiment, a plasmid comprising a minigene nucleic acid construct provided herein or a nucleic acid molecule encoding a fusion protein comprising an immunogenic polypeptide fused to one or more peptides provided herein is integrated into the Listerial chromosome using homologous recombination. Techniques for homologous recombination are well known in the art, and are described, for example, in Baloglu S, Boyle SM, et al (Immune responses of mice to vaccinia virus recombinants expressing either Listeria monocytogenes partial listeriolysin or Brucella abortus ribosomal L7/L12 protein. Vet Microbiol 2005, 109(1-2): 11-7); and Jiang L L, Song H H, et al., (Characterization of a mutant Listeria monocytogenes strain expressing green fluorescent protein. Acta Biochim Biophys Sin (Shanghai) 2005, 37(1): 19-24). In another embodiment, homologous recombination is performed as described in U.S. Pat. No. 6,855,320. In this case, a recombinant Lm strain that expresses E7 was made by chromosomal integration of the E7 gene under the control of the hly promoter and with the inclusion of the hly signal sequence to ensure secretion of the gene product, yielding the recombinant referred to as Lm-AZ/E7. In another embodiment, a temperature sensitive plasmid is used to select the recombinants Each technique represents a separate embodiment of the present invention.

In another embodiment, the construct or nucleic acid molecule is integrated into the Listerial chromosome using transposon insertion. Techniques for transposon insertion are well known in the art, and are described, inter alia, by Sun et al. (Infection and Immunity 1990, 58: 3770-3778) in the construction of DP-L967. Transposon mutagenesis has the advantage, in another embodiment, that a stable genomic insertion mutant can be formed but the disadvantage that the position in the genome where the foreign gene has been inserted is unknown.

In one embodiment, a vector provided herein is a vector known in the art, including a plasmid or a phage vector. In another embodiment, the construct or nucleic acid molecule is integrated into the Listerial chromosome using a phage vector comprising phage integration sites (Lauer P, Chow M Y et al, Construction, characterization, and use of two Listeria monocytogenes site-specific phage integration vectors. J Bacteriol 2002; 184(15): 4177-86). In certain embodiments of this method, an integrase gene and attachment site of a bacteriophage (e.g. U153 or PSA listeriophage) is used to insert the heterologous gene into the corresponding attachment site, which may be any appropriate site in the genome (e.g. comK or the 3′ end of the arg tRNA gene). In another embodiment, endogenous prophages are cured from the attachment site utilized prior to integration of the construct or heterologous gene. In another embodiment, this method results in single-copy integrants. In another embodiment, the present invention further comprises a phage based chromosomal integration system for clinical applications, where a host strain that is auxotrophic for essential enzymes, including, but not limited to, d-alanine racemase can be used, for example Lmdal(−)dat(−). In another embodiment, in order to avoid a “phage curing step,” a phage integration system based on PSA is used. This requires, in another embodiment, continuous selection by antibiotics to maintain the integrated gene. Thus, in another embodiment, the current invention enables the establishment of a phage based chromosomal integration system that does not require selection with antibiotics. Instead, an auxotrophic host strain can be complemented.

In another embodiment, a vector provided herein is a delivery vector known in the art including a bacterial delivery vector, a viral vector delivery vector, a peptide immunotherapy delivery vector, and a DNA immunotherapy delivery vector. It will be appreciated by one skilled in the art that the term “delivery vectors” refers to a construct which is capable of delivering, and, within certain embodiments expressing, one or more neo-epitopes or peptides comprising one or more neo-epitopes in a host cell. Representative examples of such vectors include viral vectors, nucleic acid expression vectors, naked DNA, and certain eukaryotic cells (e.g., producer cells). In one embodiment, a delivery vector differs from a plasmid or phage vector. In another embodiment, a delivery vector and a plasmid or phage vector of this invention are the same. In another embodiment, a delivery vector used in the methods and compositions disclosed herein is a Listeria monocytogenes strain.

In one embodiment of the methods and compositions as provided herein, the term “recombination site” or “site-specific recombination site” refers to a sequence of bases in a nucleic acid molecule that is recognized by a recombinase (along with associated proteins, in some cases) that mediates exchange or excision of the nucleic acid segments flanking the recombination sites. The recombinases and associated proteins are collectively referred to as “recombination proteins” see, e.g., Landy, A., (Current Opinion in Genetics & Development) 3:699-707; 1993).

A “phage expression vector,” “phage vector,” or “phagemid” refers to any phage-based recombinant expression system for the purpose of expressing a nucleic acid sequence of the methods and compositions as provided herein in vitro or in vivo, constitutively or inducibly, in any cell, including prokaryotic, yeast, fungal, plant, insect or mammalian cell. A phage expression vector typically can both reproduce in a bacterial cell and, under proper conditions, produce phage particles. The term includes linear or circular expression systems and encompasses both phage-based expression vectors that remain episomal or integrate into the host cell genome.

In one embodiment, the term “operably linked” as used herein means that the transcriptional and translational regulatory nucleic acid, is positioned relative to any coding sequences in such a manner that transcription is initiated. Generally, this will mean that the promoter and transcriptional initiation or start sequences are positioned 5′ to the coding region.

In one embodiment, an “open reading frame” or “ORF” is a portion of an organism's genome which contains a sequence of bases that could potentially encode a protein. In another embodiment, the start and stop ends of the ORF are not equivalent to the ends of the mRNA, but they are usually contained within the mRNA. In one embodiment, ORFs are located between the start-code sequence (initiation codon) and the stop-codon sequence (termination codon) of a gene. Thus, in one embodiment, a nucleic acid molecule operably integrated into a genome as an open reading frame with an endogenous polypeptide is a nucleic acid molecule that has integrated into a genome in the same open reading frame as an endogenous polypeptide.

In one embodiment, the present invention provides a fusion polypeptide comprising a linker sequence. In one embodiment, a “linker sequence” refers to an amino acid sequence that joins two heterologous polypeptides, or fragments or domains thereof. In general, as used herein, a linker is an amino acid sequence that covalently links the polypeptides to form a fusion polypeptide. A linker typically includes the amino acids translated from the remaining recombination signal after removal of a reporter gene from a display plasmid vector to create a fusion protein comprising an amino acid sequence encoded by an open reading frame and the display protein. As appreciated by one of skill in the art, the linker can comprise additional amino acids, such as glycine and other small neutral amino acids.

In one embodiment, “endogenous” as used herein describes an item that has developed or originated within the reference organism or arisen from causes within the reference organism. In another embodiment, endogenous refers to native.

“Stably maintained” refers, in another embodiment, to maintenance of a nucleic acid molecule or plasmid in the absence of selection (e.g. antibiotic selection) for 10 generations, without detectable loss. In another embodiment, the period is 15 generations. In another embodiment, the period is 20 generations. In another embodiment, the period is 25 generations. In another embodiment, the period is 30 generations. In another embodiment, the period is 40 generations. In another embodiment, the period is 50 generations. In another embodiment, the period is 60 generations. In another embodiment, the period is 80 generations. In another embodiment, the period is 100 generations. In another embodiment, the period is 150 generations. In another embodiment, the period is 200 generations. In another embodiment, the period is 300 generations. In another embodiment, the period is 500 generations. In another embodiment, the period is more than generations. In another embodiment, the nucleic acid molecule or plasmid is maintained stably in vitro (e.g. in culture). In another embodiment, the nucleic acid molecule or plasmid is maintained stably in vivo. In another embodiment, the nucleic acid molecule or plasmid is maintained stably both in vitro and in vitro.

In another embodiment, provided herein is a recombinant Listeria strain, comprising a nucleic acid molecule operably integrated into the Listeria genome as an open reading frame with an endogenous ActA sequence. In another embodiment, a recombinant Listeria strain of the methods and compositions as provided herein comprise an episomal expression plasmid vector comprising a nucleic acid molecule encoding fusion protein comprising an antigen fused to an ActA or a truncated ActA. In one embodiment, the expression and secretion of the antigen is under the control of an actA promoter and an actA signal sequence and it is expressed as fusion to 1-233 amino acids of ActA (truncated ActA or tActA). In another embodiment, the truncated ActA consists of the first 390 amino acids of the wild type ActA protein as described in U.S. Pat. No. 7,655,238, which is incorporated by reference herein in its entirety. In another embodiment, the truncated ActA is an ActA-N100 or a modified version thereof (referred to as ActA-N100*) in which a PEST motif has been deleted and containing the non-conservative QDNKR substitution as described in US Patent Publication Serial No. 2014/0186387.

In one embodiment, a fragment provided herein is a functional fragment. In another embodiment, a “functional fragment” is an immunogenic fragment that is capable of eliciting an immune response when administered to a subject alone or in a immunotherapy composition provided herein. In another embodiment, a functional fragment has biological activity as will be understood by a skilled artisan and as further provided herein.

In one embodiment, the Listeria strain provided herein is an attenuated strain. In another embodiment, the Listeria strain provided herein is a recombinant strain. In another embodiment, the Listeria strain provided herein is a live attenuated recombinant Listeria strain.

The recombinant Listeria strain of methods and compositions of the present invention is, in another embodiment, a recombinant Listeria monocytogenes strain. In another embodiment, the Listeria strain is a recombinant Listeria seeligeri strain. In another embodiment, the Listeria strain is a recombinant Listeria grayi strain. In another embodiment, the Listeria strain is a recombinant Listeria ivanovii strain. In another embodiment, the Listeria strain is a recombinant Listeria murrayi strain. In another embodiment, the Listeria strain is a recombinant Listeria welshimeri strain. In another embodiment, the Listeria strain is a recombinant strain of any other Listeria species known in the art.

In another embodiment, a recombinant Listeria strain of the present invention has been passaged through an animal host. In another embodiment, the passaging maximizes efficacy of the strain as a immunotherapy vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. In another embodiment, the Listeria strain contains a genomic insertion of the gene encoding the antigen-containing recombinant peptide. In another embodiment, the Listeria strain carries a plasmid comprising the gene encoding the antigen-containing recombinant peptide. In another embodiment, the passaging is performed as described herein. In another embodiment, the passaging is performed by any other method known in the art.

In another embodiment, a recombinant nucleic acid of the present invention is operably linked to a promoter/regulatory sequence that drives expression of the encoded peptide in the Listeria strain. Promoter/regulatory sequences useful for driving constitutive expression of a gene are well known in the art and include, but are not limited to, for example, the P_{hlyA}, P_{ActA}, and p60 promoters of Listeria, the Streptococcus bac promoter, the Streptomyces griseus sgiA promoter, and the B. thuringiensis phaZ promoter.

In another embodiment, inducible and tissue specific expression of the nucleic acid encoding a peptide of the present invention is accomplished by placing the nucleic acid encoding the peptide under the control of an inducible or tissue specific promoter/regulatory sequence. Examples of tissue specific or inducible promoter/regulatory sequences which are useful for this purpose include, but are not limited to the MMTV LTR inducible promoter, and the SV40 late enhancer/promoter. In another embodiment, a promoter that is induced in response to inducing agents such as metals, glucocorticoids, and the like, is utilized. Thus, it will be appreciated that the invention includes the use of any promoter/regulatory sequence, which is either known or unknown, and which is capable of driving expression of the desired protein operably linked thereto. It will be appreciated by a skilled artisan that the term “heterologous” encompasses a nucleic acid, amino acid, peptide, polypeptide, or protein derived from a different species than the reference species. Thus, for example, a Listeria strain expressing a heterologous polypeptide, in one embodiment, would express a polypeptide that is not native or endogenous to the Listeria strain, or in another embodiment, a polypeptide that is not normally expressed by the Listeria strain, or in another embodiment, a polypeptide from a source other than the Listeria strain. In another embodiment, heterologous may be used to describe something derived from a different organism within the same species. In another embodiment, the heterologous antigen is expressed by a recombinant strain of Listeria, and is processed and presented to cytotoxic T-cells upon infection of mammalian cells by the recombinant strain. In another embodiment, the heterologous antigen expressed by Listeria species need not precisely match the corresponding unmodified antigen or protein in the tumor cell or infectious agent so long as it results in a T-cell response that recognizes the unmodified antigen or protein which is naturally expressed in the mammal. The term heterologous antigen may be referred to herein as “antigenic polypeptide”, “heterologous protein”, “heterologous protein antigen”, “protein antigen”, “antigen”, and the like.

It will be appreciated by the skilled artisan that the term “episomal expression vector” encompasses a nucleic acid plasmid vector which may be linear or circular, and which is usually double-stranded in form and is extrachromosomal in that it is present in the cytoplasm of a host bacteria or cell as opposed to being integrated into the bacteria's or cell's genome. In one embodiment, an episomal expression vector comprises a gene of interest. In another embodiment, episomal vectors persist in multiple copies in the bacterial cytoplasm, resulting in amplification of the gene of interest, and, in another embodiment, viral trans-acting factors are supplied when necessary. In another embodiment, the episomal expression vector may be referred to as a plasmid herein. In another embodiment, an “integrative plasmid” comprises sequences that target its insertion or the insertion of the gene of interest carried within into a host genome. In another embodiment, an inserted gene of interest is not interrupted or subjected to regulatory constraints which often occur from integration into cellular DNA. In another embodiment, the presence of the inserted heterologous gene does not lead to rearrangement or interruption of the cell's own important regions. In another embodiment, in stable transfection procedures, the use of episomal vectors often results in higher transfection efficiency than the use of chromosome-integrating plasmids (Belt, P. B. G. M., et al (1991) Efficient cDNA cloning by direct phenotypic correction of a mutant human cell line (HPRT2) using an Epstein-Barr virus-derived cDNA expression plasmid vector. Nucleic Acids Res. 19, 4861-4866; Mazda, O., et al. (1997) Extremely efficient gene transfection into lympho-hematopoietic cell lines by Epstein-Barr virus-based vectors. J. Immunol. Methods 204, 143-151). In one embodiment, the episomal expression vectors of the methods and compositions as provided herein may be delivered to cells in vivo, ex vivo, or in vitro by any of a variety of the methods employed to deliver DNA molecules to cells. The plasmid vectors may also be delivered alone or in the form of a pharmaceutical composition that enhances delivery to cells of a subject.

In one embodiment, the term “fused” refers to operable linkage by covalent bonding. In one embodiment, the term includes recombinant fusion (of nucleic acid sequences or open reading frames thereof). In another embodiment, the term includes chemical conjugation.

“Transforming,” in one embodiment, refers to engineering a bacterial cell to take up a plasmid or other heterologous DNA molecule. In another embodiment, “transforming” refers to engineering a bacterial cell to express a gene of a plasmid or other heterologous DNA molecule. Each possibility represents a separate embodiment of the methods and compositions as provided herein.

In another embodiment, conjugation is used to introduce genetic material and/or plasmids into bacteria. Methods for conjugation are well known in the art, and are described, for example, in Nikodinovic J. et al (A second generation snp-derived Escherichia coli-Streptomyces shuttle expression vector that is generally transferable by conjugation. Plasmid. 2006 November; 56(3):223-7) and Auchtung J M et al (Regulation of a Bacillus subtilis mobile genetic element by intercellular signaling and the global DNA damage response. Proc Natl Acad Sci USA. 2005 Aug. 30; 102(35):12554-9). Each method represents a separate embodiment of the methods and compositions as provided herein.

In one embodiment, the term “attenuation,” refers to a diminution in the ability of the bacterium to cause disease in an animal. In other words, the pathogenic characteristics of the attenuated Listeria strain have been lessened compared with wild-type Listeria, although the attenuated Listeria is capable of growth and maintenance in culture. Using as an example the intravenous inoculation of Balb/c mice with an attenuated Listeria, the lethal dose at which 50% of inoculated animals survive (LD₅₀) is preferably increased above the LD₅₀ of wild-type Listeria by at least about 10-fold, more preferably by at least about 100-fold, more preferably at least about 1,000 fold, even more preferably at least about 10,000 fold, and most preferably at least about 100,000-fold. An attenuated strain of Listeria is thus one which does not kill an animal to which it is administered, or is one which kills the animal only when the number of bacteria administered is vastly greater than the number of wild type non-attenuated bacteria which would be required to kill the same animal. An attenuated bacterium should also be construed to mean one which is incapable of replication in the general environment because the nutrient required for its growth is not present therein. Thus, the bacterium is limited to replication in a controlled environment wherein the required nutrient is provided. The attenuated strains of the present invention are therefore environmentally safe in that they are incapable of uncontrolled replication.

### Compositions

In one embodiment, compositions of the present invention are immunogenic compositions. In one embodiment, compositions of the present invention induce a strong innate stimulation of interferon-gamma, which in one embodiment, has anti-angiogenic properties. In one embodiment, a Listeria of the present invention induces a strong innate stimulation of interferon-gamma, which in one embodiment, has anti-angiogenic properties (Dominiecki et al., Cancer Immunol Immunother. 2005 May; 54(5):477-88. Epub 2004 Oct. 6, incorporated herein by reference in its entirety; Beatty and Paterson, J. Immunol. 2001 Feb. 15; 166(4):2276-82, incorporated herein by reference in its entirety). In one embodiment, anti-angiogenic properties of Listeria are mediated by CD4⁺ T cells (Beatty and Paterson, 2001). In another embodiment, anti-angiogenic properties of Listeria are mediated by CD8⁺ T cells. In another embodiment, IFN-gamma secretion as a result of Listeria vaccination is mediated by NK cells, NKT cells, Th1 CD4⁺ T cells, TC1 CD8⁺ T cells, or a combination thereof.

In another embodiment, administration of compositions of the present invention induce production of one or more anti-angiogenic proteins or factors. In one embodiment, the anti-angiogenic protein is IFN-gamma. In another embodiment, the anti-angiogenic protein is pigment epithelium-derived factor (PEDF); angiostatin; endostatin; fins-like tyrosine kinase (sFlt)-1; or soluble endoglin (sEng). In one embodiment, a Listeria of the present invention is involved in the release of anti-angiogenic factors, and, therefore, in one embodiment, has a therapeutic role in addition to its role as a plasmid vector for introducing an antigen to a subject. Each Listeria strain and type thereof represents a separate embodiment of the present invention.

The immune response induced by methods and compositions as provided herein is, in another embodiment, a T cell response. In another embodiment, the immune response comprises a T cell response. In another embodiment, the response is a CD8+ T cell response. In another embodiment, the response comprises a CD8⁺ T cell response. Each possibility represents a separate embodiment as provided herein.

In another embodiment, administration of compositions of the present invention increase the number of antigen-specific T cells. In another embodiment, administration of compositions activates co-stimulatory receptors on T cells. In another embodiment, administration of compositions induces proliferation of memory and/or effector T cells. In another embodiment, administration of compositions increases proliferation of T cells. Each possibility represents a separate embodiment as provided herein.

As used throughout, the terms “composition” and “immunogenic composition” are interchangeable having all the same meanings and qualities. In one embodiment, an immunogenic composition provided herein comprising a recombinant Listeria strain and further comprising an antibody for concomitant or sequential administration of each component is also referred to as a “combination therapy”. It is to be understood by a skilled artisan that a combination therapy may also comprise additional components, antibodies, therapies, etc. The term “pharmaceutical composition” refers, in some embodiments, to a composition suitable for pharmaceutical use, for example, to administer to a subject in need. In one embodiment, the present invention provides a pharmaceutical composition comprising the attenuated Listeria strain provided herein and a pharmaceutically acceptable carrier. In another embodiment, the present invention provides a pharmaceutical composition comprising the DNA immunotherapy provided herein and a pharmaceutically acceptable carrier. In another embodiment, the present invention provides a pharmaceutical composition comprising the vaccinia virus strain or virus-like particle provided herein and a pharmaceutically acceptable carrier. In another embodiment, the present invention provides a pharmaceutical composition comprising the peptide immunotherapy provided herein and a pharmaceutically acceptable carrier.

In another embodiment, the present invention provides a recombinant immunotherapy vector comprising a nucleotide molecule of the present invention. In another embodiment, the vector is an expression vector. In another embodiment, the expression vector is a plasmid. In another embodiment, the present invention provides a method for the introduction of a nucleotide molecule of the present invention into a cell. Methods for constructing and utilizing recombinant vectors are well known in the art and are described, for example, in Sambrook et al. (2001, Molecular Cloning: A Laboratory Manual, Cold Spring Harbor Laboratory, New York), and in Brent et al. (2003, Current Protocols in Molecular Biology, John Wiley & Sons, New York). In another embodiment, the vector is a bacterial vector. In other embodiments, the vector is selected from Salmonella sp., Shigella sp., BCG, L. monocytogenes and S. gordonii. In another embodiment, one or more peptides are delivered by recombinant bacterial vectors modified to escape phagolysosomal fusion and live in the cytoplasm of the cell. In another embodiment, the vector is a viral vector. In other embodiments, the vector is selected from Vaccinia, Avipox, Adenovirus, AAV, Vaccinia virus NYVAC, Modified vaccinia strain Ankara (MVA), Semliki Forest virus, Venezuelan equine encephalitis virus, herpes viruses, and retroviruses. In another embodiment, the vector is a naked DNA vector. In another embodiment, the vector is any other vector known in the art. Each possibility represents a separate embodiment of the present invention.

Compositions of this invention may be used in methods of this invention in order to elicit an enhanced anti-tumor T cell response in a subject, in order to inhibit tumor-mediated immunosuppression in a subject, or for increasing the ratio or T effector cells to regulatory T cells (Tregs) in the spleen and tumor of a subject, or any combination thereof.

In another embodiment, a composition comprising a Listeria strain of the present invention further comprises an adjuvant. In one embodiment, a composition of the present invention further comprises an adjuvant. The adjuvant utilized in methods and compositions of the present invention is, in another embodiment, a granulocyte/macrophage colony-stimulating factor (GM-CSF) protein. In another embodiment, the adjuvant comprises a GM-CSF protein. In another embodiment, the adjuvant is a nucleotide molecule encoding GM-CSF. In another embodiment, the adjuvant comprises a nucleotide molecule encoding GM-CSF. In another embodiment, the adjuvant is saponin QS21. In another embodiment, the adjuvant comprises saponin QS21. In another embodiment, the adjuvant is monophosphoryl lipid A. In another embodiment, the adjuvant comprises monophosphoryl lipid A. In another embodiment, the adjuvant is SBAS2. In another embodiment, the adjuvant comprises SBAS2. In another embodiment, the adjuvant is an unmethylated CpG-containing oligonucleotide. In another embodiment, the adjuvant comprises an unmethylated CpG-containing oligonucleotide. In another embodiment, the adjuvant is an immune-stimulating cytokine. In another embodiment, the adjuvant comprises an immune-stimulating cytokine. In another embodiment, the adjuvant is a nucleotide molecule encoding an immune-stimulating cytokine. In another embodiment, the adjuvant comprises a nucleotide molecule encoding an immune-stimulating cytokine. In another embodiment, the adjuvant is or comprises a quill glycoside. In another embodiment, the adjuvant is or comprises a bacterial mitogen. In another embodiment, the adjuvant is or comprises a bacterial toxin. In another embodiment, the adjuvant is or comprises any other adjuvant known in the art.

In one embodiment, an immunogenic composition of this invention comprises a recombinant Listeria strain comprising a nucleic acid molecule, said nucleic acid molecule comprising a first open reading frame encoding a fusion polypeptide, wherein said fusion polypeptide comprises a truncated listeriolysin O (LLO) protein, a truncated ActA protein, or a PEST amino acid sequence fused to a heterologous antigen or fragment thereof. In another embodiment, an immunogenic composition of this invention comprises a recombinant Listeria strain comprising a nucleic acid molecule, said nucleic acid molecule comprising a first open reading frame encoding a truncated listeriolysin O (LLO) protein, a truncated ActA protein, or a PEST amino acid sequence.

In one embodiment, an immunogenic composition of this invention comprises a recombinant Listeria strain comprising a nucleic acid molecule, said nucleic acid molecule comprising a first open reading frame encoding a fusion polypeptide, wherein said fusion polypeptide comprises a truncated listeriolysin O (LLO) protein, a truncated ActA protein, or a PEST amino acid sequence fused to a heterologous antigen or fragment thereof, said composition further comprising an antibody or fragment thereof. In another embodiment said antibody or fragment thereof comprises a polyclonal antibody, a monoclonal antibody, an Fab fragment, an F(ab′)2 fragment, an Fv fragment, a single chain antibody, or any combination thereof.

In one embodiment, an immunogenic composition of this invention comprises a recombinant Listeria strain provided herein, said composition further comprising an antibody or fragment thereof. In another embodiment said antibody or fragment thereof comprises a polyclonal antibody, a monoclonal antibody, an Fab fragment, an F(ab′)2 fragment, an Fv fragment, a single chain antibody, or any combination thereof.

In another embodiment, an immunogenic composition of this invention comprises a recombinant Listeria strain, said composition further comprising an antibody or fragment thereof. In another embodiment said antibody or fragment thereof comprises a polyclonal antibody, a monoclonal antibody, an Fab fragment, an F(ab′)2 fragment, an Fv fragment, a single chain antibody, or any combination thereof.

In some embodiments, the term “antibody” refers to intact molecules as well as functional fragments thereof, also referred to herein as “antigen binding fragments”, such as Fab, F(ab′)2, and Fv that are capable of specifically interacting with a desired target as described herein, for example, blocking the binding of a checkpoint inhibitor. In another embodiment, an antibody or functional fragment thereof comprises an immune checkpoint inhibitor antagonist. In another embodiment, an antibody or functional fragment thereof comprises an anti-PD-L1/PD-L2 antibody or fragment thereof. In another embodiment, an antibody or functional fragment thereof comprises an anti-PD-1 antibody or fragment thereof. In another embodiment, an antibody or functional fragment thereof comprises an anti-CTLA-4 antibody or fragment thereof. In another embodiment, an antibody or functional fragment thereof comprises an anti-B7-H4 antibody or fragment thereof.

In some embodiments, the antibody fragments comprise: (1) Fab, the fragment which contains a monovalent antigen-binding fragment of an antibody molecule, which can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)₂ is a dimer of two Fab′ fragments held together by two disulfide bonds; (4) Fv, a genetically engineered fragment containing the variable region of the light chain and the variable region of the heavy chain expressed as two chains; or (5) Single chain antibody (“SCA”), a genetically engineered molecule containing the variable region of the light chain and the variable region of the heavy chain, linked by a suitable polypeptide linker as a genetically fused single chain molecule. Each possibility represents a separate embodiment of the present invention.

Methods of making these fragments are known in the art. (See for example, Harlow and Lane, Antibodies: A Laboratory Manual, Cold Spring Harbor Laboratory, New York, 1988, incorporated herein by reference).

In some embodiments, the antibody fragments may be prepared by proteolytic hydrolysis of the antibody or by expression in E. coli or mammalian cells (e.g. Chinese hamster ovary cell culture or other protein expression systems) of DNA encoding the fragment.

Antibody fragments can, in some embodiments, be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al., Proc. Nat'l Acad. Sci. USA 69:2659-62, 1972. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow and Filpula, Methods, 2: 97-105, 1991; Bird et al., Science 242:423-426, 1988; Pack et al., Bio/Technology 11:1271-77, 1993; and Ladner et al., U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry, Methods, 2: 106-10, 1991.

In some embodiments, the antibodies or fragments as described herein may comprise “humanized forms” of antibodies. In some embodiments, the term “humanized forms of antibodies” refers to non-human (e.g. murine) antibodies, which are chimeric molecules of immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human can be made by introducing of human immunoglobulin loci into transgenic animals, e.g. mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

In one embodiment, the disease provided herein is a cancer or a tumor. In one embodiment, the cancer treated by a method of the present invention is breast cancer. In another embodiment, the cancer is a cervical cancer. In another embodiment, the cancer is an Her2 containing cancer. In another embodiment, the cancer is a melanoma. In another embodiment, the cancer is pancreatic cancer. In another embodiment, the cancer is ovarian cancer. In another embodiment, the cancer is gastric cancer. In another embodiment, the cancer is a carcinomatous lesion of the pancreas. In another embodiment, the cancer is pulmonary adenocarcinoma. In another embodiment, the cancer is pulmonary adenocarcinoma. In another embodiment, it is a glioblastoma multiforme. In another embodiment, the cancer is colorectal adenocarcinoma. In another embodiment, the cancer is pulmonary squamous adenocarcinoma. In another embodiment, the cancer is gastric adenocarcinoma. In another embodiment, the cancer is an ovarian surface epithelial neoplasm (e.g. a benign, proliferative or malignant variety thereof). In another embodiment, the cancer is an oral squamous cell carcinoma. In another embodiment, the cancer is non-small-cell lung carcinoma. In another embodiment, the cancer is an endometrial carcinoma. In another embodiment, the cancer is a bladder cancer. In another embodiment, the cancer is a head and neck cancer. In another embodiment, the cancer is a prostate carcinoma. In another embodiment, the cancer is oropharyngeal cancer. In another embodiment, the cancer is lung cancer. In another embodiment, the cancer is anal cancer. In another embodiment, the cancer is colorectal cancer. In another embodiment, the cancer is esophageal cancer. In another embodiment, the cancer is mesothelioma.

In one embodiment, a heterologous antigen provided herein is HPV-E7. In another embodiment, the antigen is HPV-E6. In another embodiment, the HPV-E7 is from HPV strain 16. In another embodiment, the HPV-E7 is from HPV strain 18. In another embodiment, the HPV-E6 is from HPV strain 16. In another embodiment, the HPV-E7 is from HPV strain 18. In another embodiment, fragments of a heterologous antigen provided herein are also encompassed by the present invention.

In another embodiment, the antigen is Her-2/neu. In another embodiment, the antigen is NY-ESO-1. In another embodiment, the antigen is telomerase (TERT). In another embodiment, the antigen is SCCE. In another embodiment, the antigen is CEA. In another embodiment, the antigen is LMP-1. In another embodiment, the antigen is p53. In another embodiment, the antigen is carboxic anhydrase IX (CALX). In another embodiment, the antigen is PSMA. In another embodiment, the antigen is prostate stem cell antigen (PSCA). In another embodiment, the antigen is HMW-MAA. In another embodiment, the antigen is WT-1. In another embodiment, the antigen is HIV-1 Gag. In another embodiment, the antigen is Proteinase 3. In another embodiment, the antigen is Tyrosinase related protein 2. In another embodiment, the antigen is PSA (prostate-specific antigen). In another embodiment, the antigen is a bivalent PSA. In another embodiment, the antigen is an ERG. In another embodiment, the antigen is an ERG construct type III. In another embodiment, the antigen is an ERG construct type VI. In another embodiment, the antigen is an androgen receptor (AR). In another embodiment, the antigen is a PAK6. In another embodiment, the antigen comprises an epitope rich region of PAK6. In another embodiment, the antigen is selected from HPV-E7, HPV-E6, Her-2, NY-ESO-1, telomerase (TERT), SCCE, HMW-MAA, EGFR-III, survivin, baculoviral inhibitor of apoptosis repeat-containing 5 (BIRC5), WT-1, HIV-1 Gag, CEA, LMP-1, p53, PSMA, PSCA, Proteinase 3, Tyrosinase related protein 2, Muc1, PSA (prostate-specific antigen), or a combination thereof. In another embodiment, an antigen comprises the wild-type form of the antigen. In another embodiment, an antigen comprises a mutant form of the antigen.

In one embodiment, a nucleic acid sequence of PAK6 is set forth in SEQ ID NO: 102. In another embodiment, an amino acid sequence of PAK6 is set for in SEQ ID NO: 103. (See Kwek et al. (2012) J Immunol published online 5 Sep. 2012, which is incorporated herein in full.)

In another embodiment, an “immunogenic fragment” is one that elicits an immune response when administered to a subject alone or in a immunotherapy composition provided herein. Such a fragment contains, in another embodiment, the necessary epitopes in order to elicit either a humoral immune response, and/or an adaptive immune response.

In one embodiment, compositions of this invention comprise an antibody or a functional fragment thereof. In another embodiment, compositions of this invention comprise at least one antibody or functional fragment thereof. In another embodiment, a composition may comprise 2 antibodies, 3 antibodies, 4 antibodies, or more than 4 antibodies. In another embodiment, a composition of this invention comprises an Lm strain and an antibody or a functional fragment thereof. In another embodiment, a composition of this invention comprises an Lm strain and at least one antibody or a functional fragment thereof. In another embodiment, a composition of this invention comprises an Lm strain and 2 antibodies, 3 antibodies, 4 antibodies, or more than 4 antibodies. In another embodiment, a composition of this invention comprises an antibody or a functional fragment thereof, wherein the composition does not include a Listeria strain provided herein. Different antibodies present in the same or different compositions need not have the same form, for example one antibody may be a monoclonal antibody and another may be a FAb fragment. Each possibility represents a different embodiment.

In one embodiment, compositions of this invention comprise an antibody or a functional fragment thereof, which specifically binds GITR or a portion thereof. In another embodiment, compositions of this invention comprise an antibody or functional fragment thereof, which specifically binds OX40 or a portion thereof. In another embodiment, a composition may comprise an antibody that specifically bind GITR or a portion thereof, and an antibody that specifically binds OX40. In another embodiment, a composition of this invention comprises an Lm strain and an antibody or a functional fragment thereof that specifically binds GITR. In another embodiment, a composition of this invention comprises an Lm strain and an antibody or a functional fragment thereof that specifically binds OX40. In another embodiment, a composition of this invention comprises an Lm strain and an antibody that specifically binds GITR or a portion thereof, and an antibody that specifically binds OX40 or a portion thereof. In another embodiment, a composition of this invention comprises an antibody or a functional fragment thereof that specifically binds GITR, wherein the composition does not include a Listeria strain provided herein. In another embodiment, a composition of this invention comprises an antibody or a functional fragment thereof that specifically binds OX40, wherein the composition does not include a Listeria strain provided herein. In another embodiment, a composition of this invention comprises an antibody or a functional fragment thereof that specifically binds GITR, and an antibody that specifically binds GITR, wherein the composition does not include a Listeria strain provided herein. Different antibodies present in the same or different compositions need not have the same form, for example one antibody may be a monoclonal antibody and another may be a FAb fragment. Each possibility represents a different embodiment of this invention.

The term “antibody functional fragment” refers to a portion of an intact antibody that is capable of specifically binding to an antigen to cause the biological effect intended by the present invention. Examples of antibody fragments include, but are not limited to, Fab, Fab′, F(ab′)₂, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations, κ and λ light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

In one embodiment, an antibody or functional fragment thereof comprises an antigen binding region. In one embodiment, an antigen binding regions is an antibody or an antigen-binding domain thereof. In one embodiment, the antigen-binding domain thereof is a Fab or a scFv.

It will be appreciated by a skilled artisan that the term “binds” or “specifically binds,” with respect to an antibody, encompasses an antibody or functional fragment thereof, which recognizes a specific antigen, but does not substantially recognize or bind other molecules in a sample. For example, an antibody that specifically binds to an antigen from one species may also bind to that antigen from one or more species, but, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than a specific amino acid sequence.

In one embodiment, a composition of this invention comprises a recombinant Listeria monocytogenes (Lm) strain. In another embodiment, a composition of this invention comprises an antibody or functional fragment thereof, as described herein.

In one embodiment, an immunogenic composition comprises an antibody or a functional fragment thereof, provided herein, and a recombinant attenuated Listeria, provided herein. In another embodiment, each component of the immunogenic compositions provided herein is administered prior to, concurrently with, or after another component of the immunogenic compositions provided herein. In one embodiment, even when administered concurrently, an Lm composition and an antibody or functional fragment thereof may be administered as two separate compositions. Alternately, in another embodiment, an Lm composition may comprise an antibody or a functional fragment thereof.

The compositions of this invention, in another embodiment, are administered to a subject by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally.

In another embodiment, the compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present invention, the active ingredient is formulated in a capsule. In accordance with this embodiment, the compositions of the present invention comprise, in addition to the active compound and the inert carrier or diluent, a hard gelating capsule.

In another embodiment, compositions are administered by intravenous, intra-arterial, or intra-muscular injection of a liquid preparation. Suitable liquid formulations include solutions, suspensions, dispersions, emulsions, oils and the like. In one embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In some embodiments, when the antibody or functional fragment thereof is administered separately from a composition comprising a recombinant Lm strain, the antibody may be injected intravenously, subcutaneously, or directly into the tumor or tumor bed. In one embodiment, a composition comprising an antibody is injected into the space left after a tumor has been surgically removed, e.g., the space in a prostate gland following removal of a prostate tumor.

In one embodiment, the term “immunogenic composition” may encompass the recombinant Listeria provided herein, and an adjuvant, and an antibody or functional fragment thereof, or any combination thereof. In another embodiment, an immunogenic composition comprises a recombinant Listeria provided herein. In another embodiment, an immunogenic composition comprises an adjuvant known in the art or as provided herein. It is also to be understood that administration of such compositions enhance an immune response, or increase a T effector cell to regulatory T cell ratio or elicit an anti-tumor immune response, as further provided herein.

In one embodiment, this invention provides methods of use which comprise administering a composition comprising the described Listeria strains, and further comprising an antibody or functional fragment thereof. In another embodiment, methods of use comprise administering more than one antibody provided herein, which may be present in the same or a different composition, and which may be present in the same composition as the Listeria or in a separate composition. Each possibility represents a different embodiment of this invention.

In one embodiment, the term “pharmaceutical composition” encompasses a therapeutically effective amount of the active ingredient or ingredients including the Listeria strain, and at least one antibody or functional fragment thereof, together with a pharmaceutically acceptable carrier or diluent. It is to be understood that the term a “therapeutically effective amount” refers to that amount which provides a therapeutic effect for a given condition and administration regimen.

It will be understood by the skilled artisan that the term “administering” encompasses bringing a subject in contact with a composition of the present invention. In one embodiment, administration can be accomplished in vitro, i.e. in a test tube, or in vivo, i.e. in cells or tissues of living organisms, for example humans. In one embodiment, the present invention encompasses administering the Listeria strains and compositions thereof of the present invention to a subject.

The term “about” as used herein means in quantitative terms plus or minus 5%, or in another embodiment plus or minus 10%, or in another embodiment plus or minus 15%, or in another embodiment plus or minus 20%. It is to be understood by the skilled artisan that the term “subject” can encompass a mammal including an adult human or a human child, teenager or adolescent in need of therapy for, or susceptible to, a condition or its sequelae, and also may include non-human mammals such as dogs, cats, pigs, cows, sheep, goats, horses, rats, and mice. It will also be appreciated that the term may encompass livestock. The term “subject” does not exclude an individual that is normal in all respects.

Following the administration of the immunogenic compositions provided herein, the methods provided herein induce the expansion of T effector cells in peripheral lymphoid organs leading to an enhanced presence of T effector cells at the tumor site. In another embodiment, the methods provided herein induce the expansion of T effector cells in peripheral lymphoid organs leading to an enhanced presence of T effector cells at the periphery. Such expansion of T effector cells leads to an increased ratio of T effector cells to regulatory T cells in the periphery and at the tumor site without affecting the number of Tregs. It will be appreciated by the skilled artisan that peripheral lymphoid organs include, but are not limited to, the spleen, peyer's patches, the lymph nodes, the adenoids, etc. In one embodiment, the increased ratio of T effector cells to regulatory T cells occurs in the periphery without affecting the number of Tregs. In another embodiment, the increased ratio of T effector cells to regulatory T cells occurs in the periphery, the lymphoid organs and at the tumor site without affecting the number of Tregs at these sites. In another embodiment, the increased ratio of T effector cells decrease the frequency of Tregs, but not the total number of Tregs at these sites.

### Combination Therapies and Methods of Use Thereof

In one embodiment, this invention provides a method of eliciting an enhanced anti-tumor T cell response in a subject, the method comprising the step of administering to the subject an effective amount of an immunogenic composition comprising a recombinant Listeria strain comprising a nucleic acid molecule, the nucleic acid molecule comprising a first open reading frame encoding fusion polypeptide, wherein the fusion polypeptide comprises a truncated listeriolysin O (LLO) protein, a truncated ActA protein, or a PEST amino acid sequence fused to a heterologous antigen or fragment thereof, wherein said method further comprises a step of administering an effective amount of a composition comprising an immune check-point inhibitor antagonist.

In one embodiment, an immune check-point inhibitor antagonist is an anti-PD-L1/PD-L2 antibody or fragment thereof, an anti-PD-1 antibody or fragment thereof, an anti-CTLA-4 antibody or fragment thereof, or an anti-B7-H4 antibody or fragment thereof.

In another embodiment, this invention provides a method of eliciting an enhanced anti-tumor T cell response in a subject, the method comprising the step of administering to the subject an effective amount of an immunogenic composition comprising a recombinant Listeria strain comprising a nucleic acid molecule, the nucleic acid molecule comprising a first open reading frame encoding a truncated listeriolysin O (LLO) protein, a truncated ActA protein, or a PEST amino acid sequence, wherein said method further comprises a step of administering an effective amount of a composition comprising an antibody or fragment thereof to said subject. In another embodiment, the antibody is an agonist antibody or antigen binding fragment thereof. In another embodiment, the antibody is an anti-TNF receptor antibody or antigen binding fragment thereof. In another embodiment, the antibody is an anti-OX40 antibody or antigen binding fragment thereof. In another embodiment, the antibody is an anti-GITR antibody or antigen binding fragment thereof. In another embodiment, said method further comprises administering additional antibodies, which may be comprise in the composition comprising said recombinant Listeria strain or may be comprised in a separate composition.

In one embodiment, any composition comprising a Listeria strain described herein may be used in the methods of this invention. In one embodiment, any composition comprising a Listeria strain and an antibody or fragment thereof, for example an antibody binding a TNF receptor super family member, or an antibody binding to a T-cell receptor co-stimulatory molecule or an antibody binding to an antigen presenting cell receptor binding a co-stimulatory molecule, as described herein, may be used in the methods of this invention. In one embodiment, any composition comprising an antibody or functional fragment thereof described herein may be used in the methods of this invention. Compositions comprising Listeria strains with and without antibodies have been described in detail above. Compositions with antibodies have also been described in detail above. In some embodiment, in a method of this invention a composition comprising an antibody or fragment thereof, for example an antibody binding to a TNF receptor super family member, or an antibody binding to a T-cell receptor co-stimulatory molecule or an antibody binding to an antigen presenting cell receptor binding a co-stimulatory molecule, may be administered prior to, concurrent with or following administration of a composition comprising a Listeria strain.

In one embodiment, repeat administrations (doses) of compositions of this invention may be undertaken immediately following the first course of treatment or after an interval of days, weeks or months to achieve tumor regression. In another embodiment, repeat doses may be undertaken immediately following the first course of treatment or after an interval of days, weeks or months to achieve suppression of tumor growth. Assessment may be determined by any of the techniques known in the art, including diagnostic methods such as imaging techniques, analysis of serum tumor markers, biopsy, or the presence, absence or amelioration of tumor associated symptoms.

In one embodiment, provided herein are methods and compositions for preventing, treating and vaccinating against a heterologous antigen-expressing tumor and inducing an immune response against sub-dominant epitopes of the heterologous antigen, while preventing an escape mutation of the tumor.

In one embodiment, the methods and compositions for preventing, treating and vaccinating against a heterologous antigen-expressing tumor comprise the use of a truncated Listeriolysin (tLLO) protein. In another embodiment, the methods and compositions provided herein comprise a recombinant Listeria overexpressing tLLO. In another embodiment, the tLLO is expressed from a plasmid within the Listeria.

In another embodiment, provided herein is a method of preventing or treating a tumor growth or cancer in a subject, the method comprising the step of administering to the subject an immunogenic composition comprising an antibody or functional fragment thereof, as described herein, and a recombinant Listeria immunotherapy strain comprising a nucleic acid molecule, the nucleic acid molecule comprising a first open reading frame encoding fusion polypeptide, wherein the fusion polypeptide comprises a truncated listeriolysin O (LLO) protein, a truncated ActA protein, or a PEST amino acid sequence fused to a heterologous antigen or fragment thereof. In another embodiment, provided herein is a method of preventing or treating a tumor growth or cancer in a subject, the method comprising the step of administering to the subject an immunogenic composition comprising an antibody or functional fragment thereof, as described herein, and a recombinant Listeria immunotherapy strain comprising a nucleic acid molecule, the nucleic acid molecule comprising a first open reading frame encoding a truncated listeriolysin O (LLO) protein, a truncated ActA protein, or a PEST amino acid sequence.

In one embodiment, the term “treating” refers to curing a disease. In another embodiment, “treating” refers to preventing a disease. In another embodiment, “treating” refers to reducing the incidence of a disease. In another embodiment, “treating” refers to ameliorating symptoms of a disease. In another embodiment, “treating” refers to increasing performance free survival or overall survival of a patient. In another embodiment, “treating” refers to stabilizing the progression of a disease. In another embodiment, “treating” refers to inducing remission. In another embodiment, “treating” refers to slowing the progression of a disease. The terms “reducing”, “suppressing” and “inhibiting” refer in another embodiment to lessening or decreasing.

In one embodiment, provided herein is a method of increasing a ratio of T effector cells to regulatory T cells (Tregs) in the spleen and tumor microenvironments of a subject, comprising administering the immunogenic composition provided herein. In another embodiment, increasing a ratio of T effector cells to regulatory T cells (Tregs) in the spleen and tumor microenvironments in a subject allows for a more profound anti-tumor response in the subject.

In another embodiment, the T effector cells comprise CD4+FoxP3− T cells. In another embodiment, the T effector cells are CD4+FoxP3− T cells. In another embodiment, the T effector cells comprise CD4+FoxP3− T cells and CD8+ T cells. In another embodiment, the T effector cells are CD4+FoxP3− T cells and CD8+ T cells. In another embodiment, the regulatory T cells is a CD4+FoxP3+ T cell.

In one embodiment, the present invention provides methods of treating, protecting against, and inducing an immune response against a tumor or a cancer, comprising the step of administering to a subject the immunogenic composition provided herein.

In one embodiment, the present invention provides a method of preventing or treating a tumor or cancer in a human subject, comprising the step of administering to the subject the immunogenic composition strain provided herein, the recombinant Listeria strain comprising a recombinant polypeptide comprising an N-terminal fragment of an LLO protein and tumor-associated antigen, whereby the recombinant Listeria strain induces an immune response against the tumor-associated antigen, thereby treating a tumor or cancer in a human subject. In another embodiment, the immune response is a T-cell response. In another embodiment, the T-cell response is a CD4+FoxP3− T cell response. In another embodiment, the T-cell response is a CD8+ T cell response. In another embodiment, the T-cell response is a CD4+FoxP3− and CD8+ T cell response. In another embodiment, the present invention provides a method of protecting a subject against a tumor or cancer, comprising the step of administering to the subject the immunogenic composition provided herein. In another embodiment, the present invention provides a method of inducing regression of a tumor in a subject, comprising the step of administering to the subject the immunogenic composition provided herein. In another embodiment, the present invention provides a method of reducing the incidence or relapse of a tumor or cancer, comprising the step of administering to the subject the immunogenic composition provided herein. In another embodiment, the present invention provides a method of suppressing the formation of a tumor in a subject, comprising the step of administering to the subject the immunogenic composition provided herein. In another embodiment, the present invention provides a method of inducing a remission of a cancer in a subject, comprising the step of administering to the subject the immunogenic composition provided herein. In one embodiment, the nucleic acid molecule comprising a first open reading frame encoding a fusion polypeptide is integrated into the Listeria genome. In another embodiment, the nucleic acid is in a plasmid in the recombinant Listeria immunotherapy strain. In another embodiment, the nucleic acid molecule is in a bacterial artificial chromosome in the recombinant Listeria immunotherapy strain.

In one embodiment, the method comprises the step of co-administering the recombinant Listeria with an additional therapy. In another embodiment, the additional therapy is surgery, chemotherapy, an immunotherapy, a radiation therapy, antibody based immunotherapy, or a combination thereof. In another embodiment, the additional therapy precedes administration of the recombinant Listeria. In another embodiment, the additional therapy follows administration of the recombinant Listeria. In another embodiment, the additional therapy is an antibody therapy. In another embodiment, the recombinant Listeria is administered in increasing doses in order to increase the T-effector cell to regulatory T cell ration and generate a more potent anti-tumor immune response. It will be appreciated by a skilled artisan that the anti-tumor immune response can be further strengthened by providing the subject having a tumor with cytokines including, but not limited to IFN-γ, TNF-α, and other cytokines known in the art to enhance cellular immune response, some of which can be found in U.S. Pat. No. 6,991,785, incorporated by reference herein.

In one embodiment, the methods provided herein further comprise the step of co-administering an immunogenic composition provided herein with an antibody or functional fragment thereof that enhances an anti-tumor immune response in said subject.

In one embodiment, the methods provided herein further comprise the step of co-administering an immunogenic composition provided herein with a indoleamine 2,3-dioxygenase (IDO) pathway inhibitor. IDO pathway inhibitors for use in the present invention include any IDO pathway inhibitor known in the art, including but not limited to, 1-methyltryptophan (1MT), 1-methyltryptophan (1MT), Necrostatin-1, Pyridoxal Isonicotinoyl Hydrazone, Ebselen, 5-Methylindole-3-carboxaldehyde, CAY10581, an anti-IDO antibody or a small molecule IDO inhibitor. In another embodiment, the compositions and methods provided herein are also used in conjunction with, prior to, or following a chemotherapeutic or radiotherapeutic regiment. In another embodiment, IDO inhibition enhances the efficiency of chemotherapeutic agents.

In another embodiment, provided herein is a method of increasing survival of a subject suffering from cancer or having a tumor, the method comprising the step of administering to the subject an immunogenic composition comprising an antibody or functional fragment thereof, as described herein, and a recombinant Listeria immunotherapy strain comprising a nucleic acid molecule, the nucleic acid molecule comprising a first open reading frame encoding fusion polypeptide, wherein the fusion polypeptide comprises a truncated listeriolysin O (LLO) protein, a truncated ActA protein, or a PEST amino acid sequence fused to a heterologous antigen or fragment thereof.

In another embodiment, provided herein is a method of increasing antigen-specific T cells in a subject suffering from cancer or having a tumor, the method comprising the step of administering to the subject an immunogenic composition comprising an antibody or functional fragment thereof, as described herein, and a recombinant Listeria immunotherapy strain comprising a nucleic acid molecule, the nucleic acid molecule comprising a first open reading frame encoding fusion polypeptide, wherein the fusion polypeptide comprises a truncated listeriolysin O (LLO) protein, a truncated ActA protein, or a PEST amino acid sequence fused to a heterologous antigen or fragment thereof. In another embodiment, provided herein is a method of increasing T cells in a subject suffering from cancer or having a tumor, the method comprising the step of administering to the subject an immunogenic composition comprising an antibody or functional fragment thereof, as described herein, and a recombinant Listeria immunotherapy strain comprising a nucleic acid molecule, the nucleic acid molecule comprising a first open reading frame encoding a truncated listeriolysin O (LLO) protein, a truncated ActA protein, or a PEST amino acid sequence.

In another embodiment, a method of present invention further comprises the step of boosting the subject with a recombinant Listeria strain or an antibody or functional fragment thereof, as provided herein. In another embodiment, the recombinant Listeria strain used in the booster inoculation is the same as the strain used in the initial “priming” inoculation. In another embodiment, the booster strain is different from the priming strain. In another embodiment, the antibody used in the booster inoculation binds the same antigen as the antibody used in the initial “priming” inoculation. In another embodiment, the booster antibody is different from the priming antibody. In another embodiment, the same doses are used in the priming and boosting inoculations. In another embodiment, a larger dose is used in the booster. In another embodiment, a smaller dose is used in the booster. In another embodiment, the methods of the present invention further comprise the step of administering to the subject a booster vaccination. In one embodiment, the booster vaccination follows a single priming vaccination. In another embodiment, a single booster vaccination is administered after the priming vaccinations. In another embodiment, two booster vaccinations are administered after the priming vaccinations. In another embodiment, three booster vaccinations are administered after the priming vaccinations. In one embodiment, the period between a prime and a boost strain is experimentally determined by the skilled artisan. In another embodiment, the period between a prime and a boost strain is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost strain is administered 8-10 weeks after the prime strain.

In another embodiment, a method of the present invention further comprises boosting the subject with an immunogenic composition comprising an attenuated Listeria strain provided herein. In another embodiment, a method of the present invention comprises the step of administering a booster dose of the immunogenic composition comprising the attenuated Listeria strain provided herein. In another embodiment, the booster dose is an alternate form of said immunogenic composition. In another embodiment, the methods of the present invention further comprise the step of administering to the subject a booster immunogenic composition. In one embodiment, the booster dose follows a single priming dose of said immunogenic composition. In another embodiment, a single booster dose is administered after the priming dose. In another embodiment, two booster doses are administered after the priming dose. In another embodiment, three booster doses are administered after the priming dose. In one embodiment, the period between a prime and a boost dose of an immunogenic composition comprising the attenuated Listeria provided herein is experimentally determined by the skilled artisan. In another embodiment, the dose is experimentally determined by a skilled artisan. In another embodiment, the period between a prime and a boost dose is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost dose is administered 8-10 weeks after the prime dose of the immunogenic composition.

Heterologous “prime boost” strategies have been effective for enhancing immune responses and protection against numerous pathogens. Schneider et al., Immunol. Rev. 170:29-38 (1999); Robinson, H. L., Nat. Rev. Immunol. 2:239-50 (2002); Gonzalo, R. M. et al., Strain 20:1226-31 (2002); Tanghe, A., Infect. Immun 69:3041-7 (2001). Providing antigen in different forms in the prime and the boost injections appears to maximize the immune response to the antigen. DNA strain priming followed by boosting with protein in adjuvant or by viral vector delivery of DNA encoding antigen appears to be the most effective way of improving antigen specific antibody and CD4+ T-cell responses or CD8+ T-cell responses respectively. Shiver J. W. et al., Nature 415: 331-5 (2002); Gilbert, S. C. et al., Strain 20:1039-45 (2002); Billaut-Mulot, O. et al., Strain 19:95-102 (2000); Sin, J. I. et al., DNA Cell Biol. 18:771-9 (1999). Recent data from monkey vaccination studies suggests that adding CRL1005 poloxamer (12 kDa, 5% POE), to DNA encoding the HIV gag antigen enhances T-cell responses when monkeys are vaccinated with an HIV gag DNA prime followed by a boost with an adenoviral vector expressing HIV gag (Ad5-gag). The cellular immune responses for a DNA/poloxamer prime followed by an Ad5-gag boost were greater than the responses induced with a DNA (without poloxamer) prime followed by Ad5-gag boost or for Ad5-gag only. Shiver, J. W. et al. Nature 415:331-5 (2002). U.S. Patent Appl. Publication No. US 2002/0165172 A1 describes simultaneous administration of a vector construct encoding an immunogenic portion of an antigen and a protein comprising the immunogenic portion of an antigen such that an immune response is generated. The document is limited to hepatitis B antigens and HIV antigens. Moreover, U.S. Pat. No. 6,500,432 is directed to methods of enhancing an immune response of nucleic acid vaccination by simultaneous administration of a polynucleotide and polypeptide of interest. According to the patent, simultaneous administration means administration of the polynucleotide and the polypeptide during the same immune response, preferably within 0-10 or 3-7 days of each other. The antigens contemplated by the patent include, among others, those of Hepatitis (all forms), HSV, HIV, CMV, EBV, RSV, VZV, HPV, polio, influenza, parasites (e.g., from the genus Plasmodium), and pathogenic bacteria (including but not limited to M. tuberculosis, M. leprae, Chlamydia, Shigella, B. burgdorferi, enterotoxigenic E. coli, S. typhosa, H. pylori, V. cholerae, B. pertussis, etc.). All of the above references are herein incorporated by reference in their entireties.

In one embodiment, a treatment protocol of the present invention is therapeutic. In another embodiment, the protocol is prophylactic. In another embodiment, the compositions of the present invention are used to protect people at risk for cancer such as breast cancer or other types of tumors because of familial genetics or other circumstances that predispose them to these types of ailments as will be understood by a skilled artisan. In another embodiment, the immunotherapies are used as a cancer immunotherapy after debulking of tumor growth by surgery, conventional chemotherapy or radiation treatment. Following such treatments, the immunotherapies of the present invention are administered so that the CTL response to the tumor antigen of the immunotherapy destroys remaining metastases and prolongs remission from the cancer. In another embodiment, immunotherapies of the present invention are used to effect the growth of previously established tumors and to kill existing tumor cells.

In some embodiments, the term “comprise” or grammatical forms thereof, refers to the inclusion of the indicated active agent, such as the Lm strains of this invention, as well as inclusion of other active agents, such as an antibody or functional fragment thereof, and pharmaceutically acceptable carriers, excipients, emollients, stabilizers, etc., as are known in the pharmaceutical industry. In some embodiments, the term “consisting essentially of” refers to a composition, whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” may refer to components, which exert a therapeutic effect via a mechanism distinct from that of the indicated active ingredient. In some embodiments, the term “consisting essentially of” may refer to components, which exert a therapeutic effect and belong to a class of compounds distinct from that of the indicated active ingredient. In some embodiments, the term “consisting essentially of” may refer to components, which exert a therapeutic effect and may be distinct from that of the indicated active ingredient, by acting via a different mechanism of action, for example. In some embodiments, the term “consisting essentially of” may refer to components which facilitate the release of the active ingredient. In some embodiments, the term “consisting” refers to a composition, which contains the active ingredient and a pharmaceutically acceptable carrier or excipient.

As used herein, the singular form “a,” “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

In one embodiment, the term “about”, refers to a deviance of between 0.0001-5% from the indicated number or range of numbers. In one embodiment, the term “about”, refers to a deviance of between 1-10% from the indicated number or range of numbers. In one embodiment, the term “about”, refers to a deviance of up to 25% from the indicated number or range of numbers.

The subject matter disclosed herein includes, but is not limited to, the following embodiments:

1. A personalized immunotherapy system created for a subject having a disease or condition, said system comprising:
a. an attenuated Listeria strain delivery vector; and
b. a plasmid vector for transforming said Listeria strain, said plasmid vector comprising a nucleic acid construct comprising one or more open reading frames encoding one or more peptides comprising one or more neo-epitopes, wherein said neo-epitope(s) comprise immunogenic epitopes present in a disease-bearing tissue or cell of said subject having said disease or condition;
wherein transforming said Listeria strain with said plasmid vector creates a personalized immunotherapy system targeted to said subject's disease or condition.
2. The system of embodiment 1, wherein said disease or condition comprises an infectious disease or a tumor or a cancer.
3. The system of embodiment 2, wherein said infectious disease comprises a viral infection.
4. The system of embodiment 2, wherein said infectious disease comprises a bacterial infection.
5. The system of any one of embodiments 1-4, wherein said one or more neo-epitopes comprise a linear neo-epitope(s), or a conformational neo-epitope(s), or any combination thereof.
6. The system of any one of embodiments 1-5, wherein said one or more neo-epitopes comprise a solvent-exposed neo-epitope(s).
7. The system of any one of embodiments 1-6, wherein the immunogenicity of said neo-epitopes was determined using an immunogenic assay analyzing increased secretion of at least one of CD25, CD44, or CD69, or any combination thereof, or an increased secretion of a cytokine selected from the group comprising IFN-γ, TNF-α, IL-1, and IL-2, upon contacting T-cells with said one or more peptides, and wherein said increase identifies said peptide to comprise one or more T-cell neo-epitopes.
8. The system of any one of embodiments 1-7, wherein said attenuated Listeria transformed with said plasmid, secretes said one or more immunogenic peptides.
9. The system of any one of embodiments 1-8, wherein said nucleic acid sequence encoding said one or more peptides comprises one or more neo-epitopes each fused to an immunogenic polypeptide or fragment thereof.
10. The system of any one of embodiments 1-9, wherein said nucleic acid sequence encoding said one or more peptides comprises a minigene nucleic acid construct, said construct comprising an open reading frame encoding a chimeric protein, wherein said chimeric protein comprises:
a. a bacterial secretion signal sequence,
b. a ubiquitin (Ub) protein,
c. said one or more peptides comprising one or more neo-epitopes,
wherein said signal sequence, said ubiquitin and said one or more peptides in (a)-(c) are operatively linked or arranged in tandem from the amino-terminus to the carboxy-terminus.
11. The system of any one of embodiments 1-10, wherein said plasmid vector is an integrative plasmid.
12. The system of any one of embodiments 1-10, wherein said plasmid vector is an extrachromosomal multicopy plasmid.
13. The system of embodiment 12, wherein following transformation said plasmid is stably maintained in said Listeria strain in the absence of antibiotic selection.
14. The system of embodiment 9, wherein said immunogenic polypeptide is a mutated Listeriolysin O (LLO) protein, a truncated LLO (tLLO) protein, a truncated ActA protein, or a PEST amino acid sequence.
15. The system of embodiment 14, wherein said tLLO protein is set forth in SEQ ID NO: 3.
16. The system of embodiment 14, wherein said ActA is set forth in SEQ ID NO: 12-13 and 15-18.
17. The system of embodiment 14, wherein said PEST amino acid sequence is selected from the sequences set forth in SEQ ID NOs: 5-10.
18. The system of embodiment 14, wherein said mutated LLO comprises a mutation in a cholesterol-binding domain (CBD).
19. The system of embodiment 18, wherein said mutation comprises a substitution of residue C484, W491, or W492 of SEQ ID NO: 2, or any combination thereof.
20. The system of embodiment 18, wherein said mutation comprises a substitution of 1-11 amino acid within the CBD as set forth in SEQ ID NO: 68 with a 1-50 amino acid non-LLO peptide, wherein said non-LLO peptide comprises a peptide comprising a neo-epitope.
21. The system of embodiment 18, wherein said mutation comprises a deletion of a 1-11 amino acid within the CBD as set forth in SEQ ID NO: 68.
22. The system of any one of embodiments 1-21, wherein said immunogenic one or more neo-epitopes are associated with said disease or condition.
23. The system of any one of embodiments 1-22, wherein said immunogenic peptides comprising one or more neo-epitopes are comprised by a heterologous or a self-antigen or a fragment thereof.
24. The system of embodiment 23, wherein said heterologous or self-antigen is a tumor-associated antigen.
25. The system of any one of embodiments 1-24, wherein said one or more neo-epitopes comprise a cancer-specific or tumor-specific epitope.
26. The system of embodiment 25, wherein said tumor-associated antigen or fragment thereof comprises a Human Papilloma Virus (HPV)-16-E6, HPV-16-E7, HPV-18-E6, HPV-18-E7, a Her/2-neu antigen, a chimeric Her2 antigen, a Prostate Specific Antigen (PSA), ERG, Androgen receptor (AR), PAK6, Prostate Stem Cell Antigen (PSCA), NY-ESO-1, a Stratum Corneum Chymotryptic Enzyme (SCCE) antigen, Wilms tumor antigen 1 (WT-1), HIV-1 Gag, human telomerase reverse transcriptase (hTERT), Proteinase 3, Tyrosinase Related Protein 2 (TRP2), High Molecular Weight Melanoma Associated Antigen (HMW-MAA), synovial sarcoma, X (SSX)-2, carcinoembryonic antigen (CEA), Melanoma-Associated Antigen E (MAGE-A, MAGE 1, MAGE2, MAGE3, MAGE4), interleukin-13 Receptor alpha (IL13-R alpha), Carbonic anhydrase IX (CALX), survivin, GP100, an angiogenic antigen, a ras protein, a p53 protein, a p97 melanoma antigen, KLH antigen, carcinoembryonic antigen (CEA), gp100, MART1 antigen, TRP-2, HSP-70, beta-HCG, or Testisin.
27. The system of any of embodiments 2 or 25, wherein said tumor or cancer comprises a breast cancer or tumor, a cervical cancer or tumor, an Her2 expressing cancer or tumor, a melanoma, a pancreatic cancer or tumor, an ovarian cancer or tumor, a gastric cancer or tumor, a carcinomatous lesion of the pancreas, a pulmonary adenocarcinoma, a glioblastoma multiforme, a colorectal adenocarcinoma, a pulmonary squamous adenocarcinoma, a gastric adenocarcinoma, an ovarian surface epithelial neoplasm, an oral squamous cell carcinoma, non-small-cell lung carcinoma, an endometrial carcinoma, a bladder cancer or tumor, a head and neck cancer or tumor, a prostate carcinoma, a renal cancer or tumor, a bone cancer or tumor, a blood cancer, or a brain cancer or tumor.
28. The system of any one of embodiments 1-24, wherein said one or more neo-epitope comprise a infectious disease-associated-specific epitope.
29. The system of embodiment 28, wherein said infections disease is an infectious viral disease.
30. The system of embodiment 28, wherein said infections disease is an infectious bacterial disease.
31. The system of embodiment 28, wherein said the infectious disease is caused by one of the following pathogens: Leishmania, Entamoeba histolytica (which causes amebiasis), Trichuris, BCG/Tuberculosis, Malaria, Plasmodium falciparum, Plasmodium malariae, Plasmodium vivax, Rotavirus, Cholera, Diptheria-Tetanus, Pertussis, Haemophilus influenzae, Hepatitis B, Human papilloma virus, Influenza seasonal), Influenza A (H1N1) Pandemic, Measles and Rubella, Mumps, Meningococcus A+C, Oral Polio Immunotherapies, mono, bi and trivalent, Pneumococcal, Rabies, Tetanus Toxoid, Yellow Fever, Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers, Arenaviruses (LCM, Junin virus, Machupo virus, Guanarito virus, Lassa Fever), Bunyaviruses (Hantaviruses, Rift Valley Fever), Flaviruses (Dengue), Filoviruses (Ebola, Marburg), Burkholderia pseudomallei, Coxiella burnetii (Q fever), Brucella species (brucellosis), Burkholderia mallei (glanders), Chlamydia psittaci (Psittacosis), Ricin toxin (from Ricinus communis), Epsilon toxin of Clostridium perfringens, Staphylococcus enterotoxin B, Typhus fever (Rickettsia prowazekii), other Rickettsias, Food- and Waterborne Pathogens, Bacteria (Diarrheagenic E. coli, Pathogenic Vibrios, Shigella species, Salmonella BCG/, Campylobacter jejuni, Yersinia enterocolitica), Viruses (Caliciviruses, Hepatitis A, West Nile Virus, LaCrosse, Calif. encephalitis, VEE, EEE, WEE, Japanese Encephalitis Virus, Kyasanur Forest Virus, Nipah virus, hantaviruses, Tickborne hemorrhagic fever viruses, Chikungunya virus, Crimean-Congo Hemorrhagic fever virus, Tickborne encephalitis viruses, Hepatitis B virus, Hepatitis C virus, Herpes Simplex virus (HSV), Human immunodeficiency virus (HIV), Human papillomavirus (HPV)), Protozoa (Cryptosporidium parvum, Cyclospora cayatanensis, Giardia lamblia, Entamoeba histolytica, Toxoplasma), Fungi (Microsporidia), Yellow fever, Tuberculosis, including drug-resistant TB, Rabies, Prions, Severe acute respiratory syndrome associated coronavirus (SARS-CoV), Coccidioides posadasii, Coccidioides immitis, Bacterial vaginosis, Chlamydia trachomatis, Cytomegalovirus, Granuloma inguinale, Hemophilus ducreyi, Neisseria gonorrhea, Treponema pallidum, Streptococcus mutans, or Trichomonas vaginalis.
32. The system of any one of embodiments 1-31, wherein said attenuated Listeria comprises a mutation in one or more endogenous genes.
33. The system of embodiment 32, wherein said endogenous gene mutation is selected from an actA gene mutation, a prfA mutation, an actA and in1B double mutation, a dal/dal gene double mutation, or a dal/dat/actA gene triple mutation, or a combination thereof.
34. The system of embodiment 33, wherein said prfA mutation is complemented by transforming said Listeria with a plasmid comprising a nucleic acid sequence encoding a PrfA comprising a D133V mutation.
35. The system of any one of embodiments 32-34, wherein said mutation comprises an inactivation, truncation, deletion, replacement or disruption of the gene or genes.
36. The system of any one of embodiments 1-35, wherein said plasmid further comprises a second nucleic acid sequence comprising an open reading frame encoding a metabolic enzyme.
37. The system of embodiment 36, wherein said metabolic enzyme encoded by said open reading frame is an alanine racemase enzyme or a D-amino acid transferase enzyme.
38. The system of any one of embodiments 1-37, wherein said Listeria is Listeria monocytogenes.
39. The system of any one of embodiments 1-38, wherein said recombinant Listeria is cultivated or cryopreserved, or any combination thereof, and administered as a form of treatment to that subject either alone or in combination with other potentially beneficial treatments for said disease.
40. The system of any one of embodiments 1-39, wherein administering said attenuated Listeria transformed with said plasmid is accomplished as part of an immunogenic composition comprising said attenuated recombinant Listeria and an adjuvant to said subject.
41. The system of embodiment 40, wherein administering said immunogenic composition further comprises the step of concomitantly or sequentially administering one or more immunogenic compositions comprising an attenuated Listeria expressing a different peptide comprising one or more neo-epitopes and an adjuvant.
42. The system of any one of embodiments 40-41, wherein said adjuvant comprises wherein said adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.
43. A process for creating a personalized immunotherapy for a subject having a disease or condition, the process comprising the steps of:
a. comparing one or more open reading frames (ORF) in nucleic acid sequences extracted from a disease-bearing biological sample with one or more ORF in nucleic acid sequences extracted from a healthy biological sample, wherein said comparing identifies one or more neo-epitopes encoded within said one or more ORF from the disease-bearing sample;
b. screening peptides comprising said one or more neo-epitopes for an immunogenic response;
c. transforming an attenuated Listeria strain with a plasmid vector comprising a nucleic acid sequence that encodes a one or more peptides comprising said one or more immunogenic neo-epitopes; and, alternatively storing said attenuated recombinant Listeria for administering to said subject at a pre-determined period or administering said attenuated recombinant Listeria strain to said subject, wherein said attenuated recombinant Listeria strain is administered as part of an immunogenic composition.
44. The process of embodiment 43, wherein said disease or condition is an infectious disease, or a tumor or a cancer.
45. The process of embodiment 44, wherein said infectious disease comprises a viral infection.
46. The process of embodiment 44, wherein said infectious disease comprise a bacterial infection.
47. The process of any one of embodiments 43-46, wherein said disease-bearing biological sample is obtained from said subject having said disease or condition.
48. The process of any one of embodiments 43-47, wherein said healthy biological sample is obtained from said subject having said disease or condition, or from a different individual of the same species.
49. The process of any one of embodiments 43-48, wherein said disease-bearing or healthy biological sample comprises a tissue, cells isolated from blood, cells isolated from sputum, cells isolated from saliva, or cells isolated from cerebro spinal fluid.
50. The process of any one of embodiments 43-49, wherein said nucleic acid sequences are determined using exome sequencing.
51. The process of any one of embodiments 43-50, wherein said nucleic acid sequences are determined using transcriptome sequencing.
52. The process of any one of embodiments 43-51, wherein said comparing comprises a use of a screening assay or screening tool and associated digital software for comparing one or more open reading frames (ORF) in nucleic acid sequences extracted from said disease-bearing biological sample with one or more ORF in nucleic acid sequences extracted from said healthy biological sample, sample,
i. wherein said associated digital software comprises access to a sequence database that allows screening of mutations within said ORF for identification of immunogenic potential of said neo-epitopes.
53. The process of any one of embodiments 43-51, wherein said screening for an immunogenic response comprises the following steps:
a. contacting a T-cell or cells with said peptide comprising one or more neo-epitopes;
b. analyzing for an immunogenic T-cell response in said cells, wherein presence of an immunogenic T-cell response identifies said peptide as an immungenic peptide.
54. The process of any one of embodiments 43-53 wherein said one or more neo-epitopes comprise linear or conformational neo-epitopes.
55. The process of any one of embodiments 43-54, wherein said one or more neo-epitopes comprise a solvent-exposed epitope.
56. The process of any one of embodiments 43-55 wherein said attenuated recombinant Listeria secretes a said one or more immunogenic peptides comprising one or more immunogenic neo-epitopes comprising a T-cell epitope.
57. The process of any one of embodiments 43-56, wherein said transforming is accomplished using a plasmid vector comprising a nucleic acid sequence that encodes said one or more immunogenic peptides comprising one or more immunogenic neo-epitopes each fused to an immunogenic polypeptide or fragment thereof.
58. The process of any one of embodiments 43-56, wherein said transforming is accomplished using a plasmid vector comprising a minigene nucleic acid construct, said construct comprising an open reading frame encoding a chimeric protein, wherein said chimeric protein comprises:
a. a bacterial secretion signal sequence,
b. a ubiquitin (Ub) protein,
c. said one or more immunogenic peptides comprising said one or more immunogenic neo-epitope,
wherein said signal sequence, said ubiquitin and said peptide in a.-c. are operatively linked or arranged in tandem from the amino-terminus to the carboxy-terminus.
59. The process of any one of embodiments 43-58, further comprising culturing and characterizing said attenuated recombinant Listeria strain to confirm expression of said one or more immunogenic peptides.
60. The process of any one of embodiments 43-59, wherein said plasmid is an integrative plasmid.
61. The process of any one of embodiments 43-59, wherein said plasmid is an extrachromosomal multicopy plasmid.
62. The process of embodiment 61, wherein said plasmid is stably maintained in said Listeria strain in the absence of antibiotic selection.
63. The process of embodiment 57, wherein said immunogenic polypeptide is a mutated Listeriolysin O (LLO) protein, a truncated LLO (tLLO) protein, a truncated ActA protein, or a PEST amino acid sequence.
64. The process of embodiment 63, wherein said tLLO protein is set forth in SEQ ID NO: 3.
65. The process of embodiment 63, wherein said actA is set forth in SEQ ID NO: 12-13 and 15-18.
66. The process of embodiment 63, wherein said PEST amino acid sequence is selected from the sequences set forth in SEQ ID NOs: 5-10
67. The process of embodiment 63, wherein said mutated LLO comprises a mutation in a cholesterol-binding domain (CBD).
68. The process of embodiment 67, wherein said mutation comprises a substitution of residue C484, W491, or W492 of SEQ ID NO: 2, or any combination thereof.
69. The process of embodiment 67, wherein said mutation comprises a substitution of 1-11 amino acid within the CBD as set forth in SEQ ID NO: 68 with a 1-50 amino acid non-LLO peptide, wherein said non-LLO peptide comprises a peptide comprising a neo-epitope.
70. The process of embodiment 67, wherein said mutation comprises a deletion of a 1-11 amino acid within the CBD as set forth in SEQ ID NO: 68.
71. The process of any one of embodiments 43-70, wherein said one or more peptides comprising said one or more neo-epitopes are comprised by a heterologous antigen or a self-antigen associated with said disease.
72. The process of embodiment 71, wherein said heterologous antigen or said self-antigen is a tumor-associated antigen or a fragment thereof.
73. The process of any one of embodiments 43-72, wherein said one or more neo-epitopes comprise a cancer-specific or tumor-specific epitope.
74. The process of embodiment 73, wherein said tumor-associated antigen or fragment thereof comprises a Human Papilloma Virus (HPV)-16-E6, HPV-16-E7, HPV-18-E6, HPV-18-E7, a Her/2-neu antigen, a chimeric Her2 antigen, a Prostate Specific Antigen (PSA), bivalent PSA, ERG, Androgen receptor (AR), PAK6, Prostate Stem Cell Antigen (PSCA), NY-ESO-1, a Stratum Corneum Chymotryptic Enzyme (SCCE) antigen, Wilms tumor antigen 1 (WT-1), HIV-1 Gag, human telomerase reverse transcriptase (hTERT), Proteinase 3, Tyrosinase Related Protein 2 (TRP2), High Molecular Weight Melanoma Associated Antigen (HMW-MAA), synovial sarcoma, X (SSX)-2, carcinoembryonic antigen (CEA), Melanoma-Associated Antigen E (MAGE-A, MAGE 1, MAGE2, MAGE3, MAGE4), interleukin-13 Receptor alpha (IL13-R alpha), Carbonic anhydrase IX (CAIX), survivin, GP100, an angiogenic antigen, a ras protein, a p53 protein, a p97 melanoma antigen, KLH antigen, carcinoembryonic antigen (CEA), gp100, MART1 antigen, TRP-2, HSP-70, beta-HCG, or Testisin.
75. The process of any one of embodiments 44 and 73, wherein said tumor or cancer comprises a breast cancer or tumor, a cervical cancer or tumor, an Her2 expressing cancer or tumor, a melanoma, a pancreatic cancer or tumor, an ovarian cancer or tumor, a gastric cancer or tumor, a carcinomatous lesion of the pancreas, a pulmonary adenocarcinoma, a glioblastoma multiforme, a colorectal adenocarcinoma, a pulmonary squamous adenocarcinoma, a gastric adenocarcinoma, an ovarian surface epithelial neoplasm, an oral squamous cell carcinoma, non-mall-cell lung carcinoma, an endometrial carcinoma, a bladder cancer or tumor, a head and neck cancer or tumor, a prostate carcinoma, a renal cancer or tumor, a bone cancer or tumor, a blood cancer, or a brain cancer or tumor.
76. The process of any one of embodiments 43-75, wherein said one or more neo-epitopes comprise an infectious disease-associated-specific epitope.
77. The process of embodiment 76, wherein said infections disease is an infectious viral disease.
78. The process of embodiment 76, wherein said infections disease is an infectious bacterial disease.
79. The process of embodiment 76, wherein said the infectious disease is caused by one of the following pathogens: Leishmania, Entamoeba histolytica (which causes amebiasis), Trichuris, BCG/Tuberculosis, Malaria, Plasmodium falciparum, Plasmodium malariae, Plasmodium vivax, Rotavirus, Cholera, Diptheria-Tetanus, Pertussis, Haemophilus influenzae, Hepatitis B, Human papilloma virus, Influenza seasonal), Influenza A (H1N1) Pandemic, Measles and Rubella, Mumps, Meningococcus A+C, Oral Polio Immunotherapies, mono, bi and trivalent, Pneumococcal, Rabies, Tetanus Toxoid, Yellow Fever, Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers, Arenaviruses (LCM, Junin virus, Machupo virus, Guanarito virus, Lassa Fever), Bunyaviruses (Hantaviruses, Rift Valley Fever), Flaviruses (Dengue), Filoviruses (Ebola, Marburg), Burkholderia pseudomallei, Coxiella burnetii (Q fever), Brucella species (brucellosis), Burkholderia mallei (glanders), Chlamydia psittaci (Psittacosis), Ricin toxin (from Ricinus communis), Epsilon toxin of Clostridium perfringens, Staphylococcus enterotoxin B, Typhus fever (Rickettsia prowazekii), other Rickettsias, Food- and Waterborne Pathogens, Bacteria (Diarrheagenic E. coli, Pathogenic Vibrios, Shigella species, Salmonella BCG/, Campylobacter jejuni, Yersinia enterocolitica), Viruses (Caliciviruses, Hepatitis A, West Nile Virus, LaCrosse, Calif. encephalitis, VEE, EEE, WEE, Japanese Encephalitis Virus, Kyasanur Forest Virus, Nipah virus, hantaviruses, Tickborne hemorrhagic fever viruses, Chikungunya virus, Crimean-Congo Hemorrhagic fever virus, Tickborne encephalitis viruses, Hepatitis B virus, Hepatitis C virus, Herpes Simplex virus (HSV), Human immunodeficiency virus (HIV), Human papillomavirus (HPV)), Protozoa (Cryptosporidium parvum, Cyclospora cayatanensis, Giardia lamblia, Entamoeba histolytica, Toxoplasma), Fungi (Microsporidia), Yellow fever, Tuberculosis, including drug-resistant TB, Rabies, Prions, Severe acute respiratory syndrome associated coronavirus (SARS-CoV), Coccidioides posadasii, Coccidioides immitis, Bacterial vaginosis, Chlamydia trachomatis, Cytomegalovirus, Granuloma inguinale, Hemophilus ducreyi, Neisseria gonorrhea, Treponema pallidum, Streptococcus mutans, or Trichomonas vaginalis.
80. The process of any one of embodiments 43-79, wherein said attenuated Listeria comprises a mutation in one or more endogenous genes.
81. The process of embodiment 80, wherein said endogenous gene mutation is selected from an actA gene mutation, a prfA mutation, an actA and in1B double mutation, a dal/dal gene double mutation, or a dal/dat/actA gene triple mutation, or a combination thereof.
82. The process of embodiment 81, wherein said prfA mutation is complemented by transforming said Listeria with a plasmid comprising a nucleic acid sequence encoding a PrfA comprising a D133V mutation.
83. The process of any one of embodiments 80-82, wherein said mutation comprises an inactivation, truncation, deletion, replacement or disruption of the gene or genes.
84. The process of any one of embodiments 43-83, wherein said plasmid further comprises a second nucleic acid sequence comprising an open reading frame encoding a metabolic enzyme.
85. The process of embodiment 84, wherein said metabolic enzyme encoded by said open reading frame is an alanine racemase enzyme or a D-amino acid transferase enzyme.
86. The process of any one of embodiments 43-85, wherein said Listeria is Listeria monocytogenes.
87. The process of any one of embodiments 43-86, further comprising administering one or more immunogenic compositions comprising a recombinant Listeria expressing a different peptide comprising one or more different neo-epitopes and an adjuvant to said subject.
88. The process of embodiment 87, wherein administering comprises concomitant administering or sequential administering.
89. The process of any one of embodiments 87-88, wherein said adjuvant comprises wherein said adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.
90. The process of any one of embodiments 43-89, further comprising administering an immune checkpoint inhibitor antagonist.
91. The process of embodiment 90, wherein said immune checkpoint inhibitor is an anti-PD-L1/PD-L2 antibody or fragment thereof, an anti-PD-1 antibody or fragment thereof, an anti-CTLA-4 antibody or fragment thereof, or an anti-B7-H4 antibody or fragment thereof.
92. The process of any one of embodiments 43-91, wherein said administering generates a personalized enhanced anti-disease, or anti-condition immune response in said subject.
93. The process of embodiment 92, wherein said immune response comprises an anti-cancer or anti-tumor response.
94. The process of embodiment 92, wherein said immune response comprises an anti-infectious disease response.
95. The process of embodiment 94, wherein said infectious disease comprises a viral infection.
96. The process of embodiment 94, wherein said infectious disease comprises a bacterial infection.
97. The process of any one of embodiments 43-96, wherein said process allows personalized treatment or prevention of said disease or condition in said subject.
98. The process of any one of embodiments 43-97, wherein said administration increases survival time in said subject having said disease or condition.
99. A recombinant attenuated Listeria strain produced by the process of any one of embodiments 43-86.
100. A system for providing a personalized immunotherapy system created for a subject having a disease or condition, said system comprising:
a. delivery vector or other vector; and optionally
b. a plasmid vector for transforming said delivery vector, said plasmid vector comprising a nucleic acid construct comprising one or more open reading frames encoding one or more peptides comprising one or more neo-epitopes, wherein said neo-epitope(s) comprise immunogenic epitopes present in a disease-bearing tissue or cell of said subject having said disease or condition.
101. The system of embodiment 100, wherein said delivery vector comprises a bacterial delivery vector.
102. The system of embodiment 100, wherein said delivery vector comprises a viral vector delivery vector.
103. The system of embodiment 100, wherein said delivery vector comprises a peptide immunotherapy delivery vector.
104. The system of embodiment 103, wherein said peptide immunotherapy delivery vector comprises a nucleic acid construct comprising one or more open reading frames encoding one or more peptides comprising one or more neo-epitopes, wherein said neo-epitope(s) comprise immunogenic epitopes present in a disease-bearing tissue or cell of said subject having said disease or condition
105. The system of embodiment 100, wherein said delivery vector comprises a DNA plasmid immunotherapy vector.
106. The system of embodiment 105, wherein said DNA plasmid immunotherapy vector delivery vector comprises a nucleic acid construct comprising one or more open reading frames encoding one or more peptides comprising one or more neo-epitopes, wherein said neo-epitope(s) comprise immunogenic epitopes present in a disease-bearing tissue or cell of said subject having said disease or condition
107. The system of embodiment 100, wherein said disease or condition comprises an infectious disease, or a tumor or a cancer.
108. The system of embodiment 107, wherein said infectious disease comprises a viral infection.
109. The system of embodiment 107, wherein said infectious disease comprises a bacterial infection.
110. The system of any one of embodiments 100-109, wherein said one or more neo-epitopes comprise a linear neo-epitope(s), or a conformational neo-epitope(s), or any combination thereof.
111. The system of any one of embodiments 100-110, wherein said one or more neo-epitopes comprise a solvent-exposed neo-epitope(s).
112. The system of any one of embodiments 100-111, wherein the immunogenicity of said neo-epitopes was determined using an immunogenic assay analyzing increased secretion of at least one of CD25, CD44, or CD69, or any combination thereof, or an increased secretion of a cytokine selected from the group comprising IFN-γ, TNF-α, IL-1, and IL-2, upon contacting T-cells with said one or more peptides, and wherein said increase identifies said peptide to comprise one or more T-cell neo-epitopes.
113. The system of any one of embodiments 100-112, wherein said delivery vector transformed with said plasmid, secretes said one or more immunogenic peptides.
114. The system of any one of embodiments 100-113, wherein said nucleic acid sequence encoding said one or more peptides comprises one or more neo-epitopes each fused to an immunogenic polypeptide or fragment thereof.
115. The system of any one of embodiments 100-113, wherein said nucleic acid sequence encoding said one or more peptides comprises a minigene nucleic acid construct, said construct comprising an open reading frame encoding a chimeric protein, wherein said chimeric protein comprises:
a. a bacterial secretion signal sequence,
b. a ubiquitin (Ub) protein,
c. said one or more peptides comprising one or more neo-epitopes,
wherein said signal sequence, said ubiquitin and said one or more peptides in (a)-(c) are operatively linked or arranged in tandem from the amino-terminus to the carboxy-terminus.
116. The system of any one of embodiments 100-115, wherein said plasmid vector is an integrative plasmid.
117. The system of any one of embodiments 100-115, wherein said plasmid vector is an extrachromosomal multicopy plasmid.
118. The system of embodiment 114, wherein said immunogenic polypeptide is a mutated Listeriolysin O (LLO) protein, a truncated LLO (tLLO) protein, a truncated ActA protein, or a PEST amino acid sequence.
119. The system of embodiment 118, wherein said tLLO protein is set forth in SEQ ID NO: 3.
120. The system of embodiment 118, wherein said ActA is set forth in SEQ ID NO: 12-13 and 15-18.
121. The system of embodiment 118, wherein said PEST amino acid sequence is selected from the sequences set forth in SEQ ID NOs: 5-10.
122. The system of embodiment 118, wherein said mutated LLO comprises a mutation in a cholesterol-binding domain (CBD).
123. The system of embodiment 118, wherein said mutation comprises a substitution of residue C484, W491, or W492 of SEQ ID NO: 2, or any combination thereof.
124. The system of embodiment 118, wherein said mutation comprises a substitution of 1-11 amino acid within the CBD as set forth in SEQ ID NO: 68 with a 1-50 amino acid non-LLO peptide, wherein said non-LLO peptide comprises a peptide comprising a neo-epitope.
125. The system of embodiment 118, wherein said mutation comprises a deletion of a 1-11 amino acid within the CBD as set forth in SEQ ID NO: 68.
126. The system of any one of embodiments 100-125, wherein said immunogenic one or more neo-epitopes are associated with said disease or condition.
127. The system of any one of embodiments 100-125, wherein said immunogenic peptides comprising one or more neo-epitopes are comprised by a heterologous or a self-antigen or a fragment thereof.
128. The system of embodiment 127, wherein said heterologous or self-antigen is a tumor-associated antigen.
129. The system of any one of embodiments 100-128, wherein said one or more neo-epitopes comprise a cancer-specific or tumor-specific epitope.
130. The system of embodiment 128, wherein said tumor-associated antigen or fragment thereof comprises a Human Papilloma Virus (HPV)-16-E6, HPV-16-E7, HPV-18-E6, HPV-18-E7, a Her/2-neu antigen, a chimeric Her2 antigen, a Prostate Specific Antigen (PSA), ERG, Androgen receptor (AR), PAK6, Prostate Stem Cell Antigen (PSCA), NY-ESO-1, a Stratum Corneum Chymotryptic Enzyme (SCCE) antigen, Wilms tumor antigen 1 (WT-1), HIV-1 Gag, human telomerase reverse transcriptase (hTERT), Proteinase 3, Tyrosinase Related Protein 2 (TRP2), High Molecular Weight Melanoma Associated Antigen (HMW-MAA), synovial sarcoma, X (SSX)-2, carcinoembryonic antigen (CEA), Melanoma-Associated Antigen E (MAGE-A, MAGE 1, MAGE2, MAGE3, MAGE4), interleukin-13 Receptor alpha (IL13-R alpha), Carbonic anhydrase IX (CALX), survivin, GP100, an angiogenic antigen, a ras protein, a p53 protein, a p97 melanoma antigen, KLH antigen, carcinoembryonic antigen (CEA), gp100, MART1 antigen, TRP-2, HSP-70, beta-HCG, or Testisin.
131. The system of any of embodiments 107 or 128, wherein said tumor or cancer comprises a breast cancer or tumor, a cervical cancer or tumor, an Her2 expressing cancer or tumor, a melanoma, a pancreatic cancer or tumor, an ovarian cancer or tumor, a gastric cancer or tumor, a carcinomatous lesion of the pancreas, a pulmonary adenocarcinoma, a glioblastoma multiforme, a colorectal adenocarcinoma, a pulmonary squamous adenocarcinoma, a gastric adenocarcinoma, an ovarian surface epithelial neoplasm, an oral squamous cell carcinoma, non-small-cell lung carcinoma, an endometrial carcinoma, a bladder cancer or tumor, a head and neck cancer or tumor, a prostate carcinoma, a renal cancer or tumor, a bone cancer or tumor, a blood cancer, or a brain cancer or tumor.
132. The system of any one of embodiments 100-131, wherein said one or more neo-epitope comprise a infectious disease-associated-specific epitope.
133. The system of embodiment 132, wherein said infections disease is an infectious viral disease.
134. The system of embodiment 132, wherein said infections disease is an infectious bacterial disease.
135. The system of embodiment 132, wherein said the infectious disease is caused by one of the following pathogens: Leishmania, Entamoeba histolytica (which causes amebiasis), Trichuris, BCG/Tuberculosis, Malaria, Plasmodium falciparum, Plasmodium malariae, Plasmodium vivax, Rotavirus, Cholera, Diptheria-Tetanus, Pertussis, Haemophilus influenzae, Hepatitis B, Human papilloma virus, Influenza seasonal), Influenza A (H1N1) Pandemic, Measles and Rubella, Mumps, Meningococcus A+C, Oral Polio Immunotherapies, mono, bi and trivalent, Pneumococcal, Rabies, Tetanus Toxoid, Yellow Fever, Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers, Arenaviruses (LCM, Junin virus, Machupo virus, Guanarito virus, Lassa Fever), Bunyaviruses (Hantaviruses, Rift Valley Fever), Flaviruses (Dengue), Filoviruses (Ebola, Marburg), Burkholderia pseudomallei, Coxiella burnetii (Q fever), Brucella species (brucellosis), Burkholderia mallei (glanders), Chlamydia psittaci (Psittacosis), Ricin toxin (from Ricinus communis), Epsilon toxin of Clostridium perfringens, Staphylococcus enterotoxin B, Typhus fever (Rickettsia prowazekii), other Rickettsias, Food- and Waterborne Pathogens, Bacteria (Diarrheagenic E. coli, Pathogenic Vibrios, Shigella species, Salmonella BCG/, Campylobacter jejuni, Yersinia enterocolitica), Viruses (Caliciviruses, Hepatitis A, West Nile Virus, LaCrosse, Calif. encephalitis, VEE, EEE, WEE, Japanese Encephalitis Virus, Kyasanur Forest Virus, Nipah virus, hantaviruses, Tickborne hemorrhagic fever viruses, Chikungunya virus, Crimean-Congo Hemorrhagic fever virus, Tickborne encephalitis viruses, Hepatitis B virus, Hepatitis C virus, Herpes Simplex virus (HSV), Human immunodeficiency virus (HIV), Human papillomavirus (HPV)), Protozoa (Cryptosporidium parvum, Cyclospora cayatanensis, Giardia lamblia, Entamoeba histolytica, Toxoplasma), Fungi (Microsporidia), Yellow fever, Tuberculosis, including drug-resistant TB, Rabies, Prions, Severe acute respiratory syndrome associated coronavirus (SARS-CoV), Coccidioides posadasii, Coccidioides immitis, Bacterial vaginosis, Chlamydia trachomatis, Cytomegalovirus, Granuloma inguinale, Hemophilus ducreyi, Neisseria gonorrhea, Treponema pallidum, Streptococcus mutans, or Trichomonas vaginalis.
136. The system of any one of embodiments 100-135, wherein said delivery vector is cultivated, or cryopreserved, or any combination thereof, and administered as a form of treatment to that subject either alone or in combination with other potentially beneficial treatments for said disease.
137. The system of any one of embodiments 100-136, wherein administering said delivery vector optionally transformed with said plasmid is accomplished as part of an immunogenic composition comprising said recombinant delivery vector and an adjuvant to said subject.
138. The system of embodiment 137, wherein administering said immunogenic composition further comprises the step of concomitantly or sequentially administering one or more immunogenic compositions comprising a delivery vector expressing a different peptide comprising one or more neo-epitopes and an adjuvant.
139. The system of any one of embodiments 137-138, wherein said adjuvant comprises wherein said adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.
140. A process for creating a personalized immunotherapy for a subject having a disease or condition, the process comprising the steps of:
a. comparing one or more open reading frames (ORF) in nucleic acid sequences extracted from a disease-bearing biological sample with one or more ORF in nucleic acid sequences extracted from a healthy biological sample, wherein said comparing identifies one or more nucleic acid sequences encoding one or more peptides comprising one or more neo-epitopes encoded within said one or more ORF from the disease-bearing sample;
b. transforming an attenuated Listeria strain with a vector comprising a nucleic acid sequence encoding one or more peptides comprising said one or more neo-epitopes identified in a.; and, alternatively storing said attenuated recombinant Listeria for administering to said subject at a pre-determined period or administering a composition comprising said attenuated recombinant Listeria strain to said subject, and wherein said administering results in the generation of a personalized T-cell immune response against said disease or said condition; optionally,
c. Obtaining a second biological sample from said subject comprising a T-cell clone or T-infiltrating cell from said T-cell immune response and characterizing specific peptides comprising one or more neo-epitopes bound by MHC Class I or MHC Class II molecules on said T cells, wherein said one or more neo-epitopes are immunogenic;
d. Screening for and selecting a nucleic acid construct encoding one or more peptides comprising one or more immunogenic neo-epitope identified in c.; and,
e. Transforming a second attenuated recombinant Listeria strain with a vector comprising a nucleic acid sequence encoding one or more peptides comprising said one or more immunogenic neo-epitopes; and, alternatively storing said second attenuated recombinant Listeria for administering to said subject at a pre-determined period or administering a second composition comprising said second attenuated recombinant Listeria strain to said subject,

wherein said process creates a personalized immunotherapy for said subject.

141. The process of embodiment 140, wherein said comparing comprises a use of a screening assay or screening tool and associated digital software for comparing one or more ORF in nucleic acid sequences extracted from said disease-bearing biological sample with one or more ORF in nucleic acid sequences extracted from said healthy biological sample,
i. wherein said associated digital software comprises access to a sequence database that allows screening of mutations within said ORF in said nucleic acid sequences extracted from said disease-bearing biological sample for identification of immunogenic potential of said neo-epitopes.
142. The process of any one of embodiments 140-141, wherein the process of obtaining a second biological sample from said subject comprises obtaining a biological sample comprising T-cell clones or T-infiltrating cells that expand following administration of said second composition comprising said attenuated recombinant Listeria strain.
143. The process of any one of embodiments 140-142, wherein said biological sample is tissue, cells, blood or sera.
144. The process of any one of embodiments 140-143, wherein the process of characterizing comprises the steps of:
i. Identifying, isolating and expanding T cell clones or T-infiltrating cells that respond against said disease;
ii. Screening for and identifying one or more peptides comprising one or more immunogenic neo-epitopes loaded on specific MHC Class I or MHC Class II molecules to which a T-cell receptor on said T cells binds.
145. The process of embodiment 144, wherein said screening for and identifying comprises T-cell receptor sequencing, multiplex based flow cytometry, or high-performance liquid chromatography.
146. The process of embodiment 145, wherein said sequencing comprises the use of associated digital software and database.
147. The process of any one of embodiments 140-146, wherein said disease or condition is an infectious disease, or a tumor or a cancer.
148. The process of embodiment 147, wherein said infectious disease comprises a viral or bacterial infection.
149. The process of any one of embodiments 140-148, wherein said disease-bearing biological sample is obtained from said subject having said disease or condition.
150. The process of any one of embodiments 140-149, wherein said healthy biological sample is obtained from said subject having said disease or condition.
151. The process of any one of embodiments 140-150, wherein said sequencing of said nucleic acid sequences are determined using exome sequencing or transcriptome sequencing.
152. The process of any one of embodiments 140-151, wherein said one or more neo-epitopes comprise linear neo-epitopes.
153. The process of any one of embodiments 140-152, wherein said one or more neo-epitopes comprise a solvent-exposed epitope.
154. The process of any one of embodiments 140-153, wherein said attenuated recombinant Listeria secretes said one or more peptides comprising one or more immunogenic neo-epitopes.
155. The process of any one of claims 140-154, wherein said one or more immunogenic neo-epitopes comprise a T-cell epitope.
156. The process of any one of embodiments 140-155, wherein said transforming is accomplished using a plasmid or phage vector.
157. The process of any one of embodiments 140-156, wherein said one or more peptides comprising one or more immunogenic neo-epitopes are each fused to an immunogenic polypeptide or fragment thereof.
158. The process of any one of embodiments 140-157, wherein said transforming is accomplished using a plasmid vector comprising a minigene nucleic acid construct, said construct comprising one or more open reading frames encoding a chimeric protein, wherein said chimeric protein comprises:
a. a bacterial secretion signal sequence,
b. a ubiquitin (Ub) protein,
c. said one or more peptides comprising said one or more neo-epitopes,
wherein said signal sequence, said ubiquitin and said one or more peptides in a.-c. are operatively linked or arranged in tandem from the amino-terminus to the carboxy-terminus.
159. The process of any one of embodiments 140-158, further comprising culturing and characterizing said attenuated recombinant Listeria strain to confirm expression and secretion of said one or more peptides.
160. The process of any one of embodiments 156-158, wherein said plasmid is an integrative plasmid.
161. The process of any one of embodiments 156-158, wherein said plasmid is an extrachromosomal multicopy plasmid.
162. The process of embodiment 156-158, wherein said plasmid is stably maintained in said Listeria strain in the absence of antibiotic selection.
163. The process of embodiment 157, wherein said immunogenic polypeptide is a mutated Listeriolysin O (LLO) protein, a truncated LLO (tLLO) protein, a truncated ActA protein, or a PEST amino acid sequence.
164. The process of embodiment 163, wherein said tLLO protein is set forth in SEQ ID NO: 3.
165. The process of embodiment 163, wherein said actA is set forth in SEQ ID NO: 12-13 and 15-18.
166. The process of embodiment 163, wherein said PEST amino acid sequence is selected from the sequences set forth in SEQ ID NOs: 5-10.
167. The process of embodiment 163, wherein said mutated LLO comprises a mutation in a cholesterol-binding domain (CBD).
168. The process of embodiment 167, wherein said mutation comprises a substitution of residue C484, W491, or W492 of SEQ ID NO: 2, or any combination thereof.
169. The process of embodiment 167, wherein said mutation comprises a substitution of 1-11 amino acid within the CBD set forth in SEQ ID NO: 68 with a 1-50 amino acid non-LLO peptide, wherein said non-LLO peptide comprises a peptide comprising a neo-epitope.
170. The process of embodiment 167, wherein said mutation comprises a deletion of a 1-11 amino acid within the CBD as set forth in SEQ ID NO: 68.
171. The process of any one of embodiments 140-170, wherein said one or more peptides are comprised by a heterologous antigen or a self-antigen associated with said disease.
172. The process of embodiment 171, wherein said heterologous antigen or said self-antigen is a tumor-associated antigen or a fragment thereof.
173. The process of any one of embodiments 140-172, wherein said one or more neo-epitopes comprise a cancer-specific or tumor-specific epitope.
174. The process of any one of embodiments 171-173, wherein said tumor-associated antigen or fragment thereof comprises a Human Papilloma Virus (HPV)-16-E6, HPV-16-E7, HPV-18-E6, HPV-18-E7, a Her/2-neu antigen, a chimeric Her2 antigen, a Prostate Specific Antigen (PSA), bivalent PSA, ERG, Androgen receptor (AR), PAK6, Prostate Stem Cell Antigen (PSCA), NY-ESO-1, a Stratum Corneum Chymotryptic Enzyme (SCCE) antigen, Wilms tumor antigen 1 (WT-1), HIV-1 Gag, human telomerase reverse transcriptase (hTERT), Proteinase 3, Tyrosinase Related Protein 2 (TRP2), High Molecular Weight Melanoma Associated Antigen (HMW-MAA), synovial sarcoma, X (SSX)-2, carcinoembryonic antigen (CEA), Melanoma-Associated Antigen E (MAGE-A, MAGE 1, MAGE2, MAGE3, MAGE4), interleukin-13 Receptor alpha (IL13-R alpha), Carbonic anhydrase IX (CAIX), survivin, GP100, an angiogenic antigen, a ras protein, a p53 protein, a p97 melanoma antigen, KLH antigen, carcinoembryonic antigen (CEA), gp100, MART1 antigen, TRP-2, HSP-70, beta-HCG, or Testisin.
175. The process of any one of embodiments 147 and 173, wherein said tumor or cancer comprises a breast cancer or tumor, a cervical cancer or tumor, an Her2 expressing cancer or tumor, a melanoma, a pancreatic cancer or tumor, an ovarian cancer or tumor, a gastric cancer or tumor, a carcinomatous lesion of the pancreas, a pulmonary adenocarcinoma, a glioblastoma multiforme, a colorectal adenocarcinoma, a pulmonary squamous adenocarcinoma, a gastric adenocarcinoma, an ovarian surface epithelial neoplasm, an oral squamous cell carcinoma, non-mall-cell lung carcinoma, an endometrial carcinoma, a bladder cancer or tumor, a head and neck cancer or tumor, a prostate carcinoma, a renal cancer or tumor, a bone cancer or tumor, a blood cancer, or a brain cancer or tumor.
176. The process of any one of embodiments 140-175, wherein said one or more neo-epitopes comprise an infectious disease-associated-specific epitope.
177. The process of embodiment 176, wherein said infections disease is an infectious viral disease.
178. The process of embodiment 176, wherein said infections disease is an infectious bacterial disease.
179. The process of embodiment 176, wherein said the infectious disease is caused by one of the following pathogens: Leishmania, Entamoeba histolytica (which causes amebiasis), Trichuris, BCG/Tuberculosis, Malaria, Plasmodium falciparum, Plasmodium malariae, Plasmodium vivax, Rotavirus, Cholera, Diptheria-Tetanus, Pertussis, Haemophilus influenzae, Hepatitis B, Human papilloma virus, Influenza seasonal), Influenza A (H1N1) Pandemic, Measles and Rubella, Mumps, Meningococcus A+C, Oral Polio Immunotherapies, mono, bi and trivalent, Pneumococcal, Rabies, Tetanus Toxoid, Yellow Fever, Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers, Arenaviruses (LCM, Junin virus, Machupo virus, Guanarito virus, Lassa Fever), Bunyaviruses (Hantaviruses, Rift Valley Fever), Flaviruses (Dengue), Filoviruses (Ebola, Marburg), Burkholderia pseudomallei, Coxiella burnetii (Q fever), Brucella species (brucellosis), Burkholderia mallei (glanders), Chlamydia psittaci (Psittacosis), Ricin toxin (from Ricinus communis), Epsilon toxin of Clostridium perfringens, Staphylococcus enterotoxin B, Typhus fever (Rickettsia prowazekii), other Rickettsias, Food- and Waterborne Pathogens, Bacteria (Diarrheagenic E. coli, Pathogenic Vibrios, Shigella species, Salmonella BCG/, Campylobacter jejuni, Yersinia enterocolitica), Viruses (Caliciviruses, Hepatitis A, West Nile Virus, LaCrosse, Calif. encephalitis, VEE, EEE, WEE, Japanese Encephalitis Virus, Kyasanur Forest Virus, Nipah virus, hantaviruses, Tickborne hemorrhagic fever viruses, Chikungunya virus, Crimean-Congo Hemorrhagic fever virus, Tickborne encephalitis viruses, Hepatitis B virus, Hepatitis C virus, Herpes Simplex virus (HSV), Human immunodeficiency virus (HIV), Human papillomavirus (HPV)), Protozoa (Cryptosporidium parvum, Cyclospora cayatanensis, Giardia lamblia, Entamoeba histolytica, Toxoplasma), Fungi (Microsporidia), Yellow fever, Tuberculosis, including drug-resistant TB, Rabies, Prions, Severe acute respiratory syndrome associated coronavirus (SARS-CoV), Coccidioides posadasii, Coccidioides immitis, Bacterial vaginosis, Chlamydia trachomatis, Cytomegalovirus, Granuloma inguinale, Hemophilus ducreyi, Neisseria gonorrhea, Treponema pallidum, Streptococcus mutans, or Trichomonas vaginalis.
180. The process of any one of embodiments 140-179, wherein said attenuated Listeria comprises a mutation in one or more endogenous genes.
181. The process of embodiment 180, wherein said endogenous gene mutation is selected from an actA gene mutation, a prfA mutation, an actA and in1B double mutation, a dal/dal gene double mutation, or a dal/dat/actA gene triple mutation, or a combination thereof.
182. The process of any one of embodiments 180-181, wherein said mutation comprises an inactivation, truncation, deletion, replacement or disruption of the gene or genes.
183. The process of any one of embodiments 140-182, wherein said vector further comprises an open reading frame or a second nucleic acid sequence comprising an open reading frame encoding a metabolic enzyme.
184. The process of embodiment 183, wherein said metabolic enzyme encoded by said open reading frame is an alanine racemase enzyme or a D-amino acid transferase enzyme.
185. The process of any one of embodiments 140-184, wherein said Listeria is Listeria monocytogenes.
186. The process of any one of embodiments 140-185, further comprising administering an adjuvant to said subject.
187. The process of embodiment 176, wherein said adjuvant comprises wherein said adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.
188. The process of any one of embodiments 140-187, further comprising administering an immune checkpoint inhibitor antagonist.
189. The process of embodiment 188, wherein said immune checkpoint inhibitor is an anti-PD-L1/PD-L2 antibody or fragment thereof, an anti-PD-1 antibody or fragment thereof, an anti-CTLA-4 antibody or fragment thereof, or an anti-B7-H4 antibody or fragment thereof.
190. The process of any one of embodiments 140-189, wherein said administering generates a personalized enhanced anti-disease, or anti-condition immune response in said subject.
191. The process of embodiment 190, wherein said immune response comprises an anti-cancer or anti-tumor response.
192. The process of embodiment 190, wherein said immune response comprises an anti-infectious disease response.
193. The process of embodiment 192, wherein said infectious disease comprises a viral infection.
194. The process of embodiment 192, wherein said infectious disease comprises a bacterial infection.
195. The process of any one of embodiments 140-194, wherein said process allows personalized treatment or prevention of said disease or condition in said subject.
196. The process of any one of embodiments 140-191, wherein said personalized immunotherapy increases survival time in said subject having said disease or condition.
197. A recombinant attenuated Listeria strain produced by the process of any one of embodiments 140-185.
198. A process for creating a personalized immunotherapy for a subject having a disease or condition, the process comprising the steps of:
a. comparing one or more open reading frames (ORF) in nucleic acid sequences extracted from a disease-bearing biological sample with one or more ORF in nucleic acid sequences extracted from a healthy biological sample, wherein said comparing identifies one or more nucleic acid sequences encoding one or more peptides comprising one or more neo-epitopes encoded within said one or more ORF from the disease-bearing sample;
b. transforming a vector with a nucleic acid sequence encoding one or more peptides comprising said one or more neo-epitopes identified in a., or generating a DNA immunotherapy vector or a peptide immunotherapy vector using said nucleic acid sequence encoding one or more peptides comprising said one or more neo-epitopes identified in a.; and, alternatively storing said vector or said DNA immunotherapy or said peptide immunotherapy for administering to said subject at a pre-determined period or administering a composition comprising said vector, said DNA immunotherapy or said peptide immunotherapy to said subject, and wherein said administering results in the generation of a personalized T-cell immune response against said disease or said condition; and optionally,
c. Obtaining a second biological sample from said subject comprising a T-cell clone or T-infiltrating cell from said T-cell immune response and characterizing specific peptides comprising one or more immunogenic neo-epitopes bound by MHC Class I or MHC Class II molecules on said T cells;
d. Screening for and selecting a nucleic acid construct encoding one or more peptides comprising one or more immunogenic neo-epitope identified in c.; and,
e. Transforming a second vector with a nucleic acid sequence comprising one or more open reading frames encoding one or more peptides comprising said one or more immunogenic neo-epitopes or generating a DNA immunotherapy vector or a peptide immunotherapy vector using said nucleic acid sequence encoding one or more peptides comprising said one or more immunogenic neo-epitopes identified in c.; and, alternatively storing said vector or said DNA immunotherapy or said peptide immunotherapy for administering to said subject at a pre-determined period, or administering a composition comprising said vector, said DNA immunotherapy or said peptide immunotherapy to said subject,

wherein said process creates a personalized immunotherapy for said subject.

199. The process of embodiment 198, wherein said comparing comprises a use of a screening assay or screening tool and associated digital software for comparing one or more ORF in nucleic acid sequences extracted from said disease-bearing biological sample with one or more ORF in nucleic acid sequences extracted from said healthy biological sample, ii. wherein said associated digital software comprises access to a sequence database that allows screening of mutations within said ORF in said nucleic acid sequences extracted from said disease-bearing biological sample for identification of immunogenic potential of said neo-epitopes.
200. The process of any one of embodiments 198-199, wherein the process of obtaining a second biological sample from said subject comprises obtaining a second biological sample comprising T-cell clones or T-infiltrating cells that expand following administration of said second composition comprising said vector, said DNA immunotherapy or said peptide immunotherapy.
201. The process of any one of embodiments 198-200, wherein said biological sample is tissue, cells, blood or sera.
202. The process of any one of embodiments 198-201, wherein the process of characterizing comprises the steps of:
i. Identifying, isolating and expanding T cell clones or T-infiltrating cells that respond against said disease;
ii. Screening for and identifying one or more peptides comprising one or more immunogenic neo-epitopes loaded on specific MHC Class I or MHC Class II molecules to which a T-cell receptor on said T cells binds.
203. The process of embodiment 202, wherein said screening for and identifying comprises T-cell receptor sequencing, multiplex based flow cytometry, or high-performance liquid chromatography.
204. The process of embodiment 203, wherein said sequencing comprises the use of associated digital software and database.
205. The process of any one of embodiments 198-204 wherein said disease or condition is an infectious disease, or a tumor or a cancer.
206. The process of embodiment 205, wherein said infectious disease comprises a viral or bacterial infection.
207. The process of any one of embodiments 198-206, wherein said disease-bearing biological sample is obtained from said subject having said disease or condition.
208. The process of any one of embodiments 198-207, wherein said healthy biological sample is obtained from said subject having said disease or condition.
209. The process of any one of embodiments 198-208, wherein said sequencing of said nucleic acid sequences are determined using exome sequencing or transcriptome sequencing.
210. The process of any one of embodiments 198-209, wherein said one or more neo-epitopes comprise linear neo-epitopes.
211. The process of any one of embodiments 198-210, wherein said one or more neo-epitopes comprise a solvent-exposed epitope.
212. The process of any one of embodiments 198-211, wherein said one or more immunogenic neo-epitopes comprise a T-cell epitope.
213. The process of any one of embodiments 198-212, wherein said vector is a vaccinia virus or a virus-like particle.
214. The process of any one of embodiments 198-213, further comprising culturing and characterizing said vaccinia virus or virus-like particle to confirm expression of said one or more peptides.
215. The process of any one of embodiments 198-212, wherein said DNA immunotherapy comprises a nucleic acid sequence comprising one or more ORF encoding one or more peptides comprising one or more immunogenic neo-epitopes.
216. The process of embodiment 215, wherein said nucleic acid sequence is in the form of a plasmid.
217. The process of any one of embodiments 216, wherein said plasmid is an integrative or an extrachrosomomal multicopy plasmid.
218. The process of any one of embodiments 59-78, wherein said one or more peptides comprising one or more immunogenic neo-epitopes are each fused to an immunogenic polypeptide or fragment thereof.
219. The process of any one of embodiments 198-212, wherein said peptide immunotherapy comprises one or more peptides comprising one or more immunogenic neo-epitopes, wherein each peptide is fused to or mixed with an immunogenic polypeptide or fragment thereof.
220. The process of any one of embodiments 218-219, wherein said immunogenic polypeptide is a mutated Listeriolysin O (LLO) protein, a truncated LLO (tLLO) protein, a truncated ActA protein, or a PEST amino acid sequence.
221. The process of embodiment 220, wherein said tLLO protein is set forth in SEQ ID NO: 3.
222. The process of embodiment 220, wherein said actA is set forth in SEQ ID NO: 12-13 and 15-18.
223. The process of embodiment 220, wherein said PEST amino acid sequence is selected from the sequences set forth in SEQ ID NOs: 5-10.
224. The process of embodiment 220, wherein said mutated LLO comprises a mutation in a cholesterol-binding domain (CBD).
225. The process of embodiment 224, wherein said mutation comprises a substitution of residue C484, W491, or W492 of SEQ ID NO: 2, or any combination thereof.
226. The process of embodiment 224, wherein said mutation comprises a substitution of 1-11 amino acid within the CBD set forth in SEQ ID NO: 68 with a 1-50 amino acid non-LLO peptide, wherein said non-LLO peptide comprises a peptide comprising a neo-epitope.
227. The process of embodiment 224, wherein said mutation comprises a deletion of a 1-11 amino acid within the CBD as set forth in SEQ ID NO: 68.
228. The process of any one of embodiments 198-227, wherein said one or more peptides are comprised by a heterologous antigen or a self-antigen associated with said disease.
229. The process of embodiment 228, wherein said heterologous antigen or said self-antigen is a tumor-associated antigen or a fragment thereof.
230. The process of any one of embodiments 198-229, wherein said one or more neo-epitopes comprise a cancer-specific or tumor-specific epitope.
231. The process of any one of embodiments 229-230, wherein said tumor-associated antigen or fragment thereof comprises a Human Papilloma Virus (HPV)-16-E6, HPV-16-E7, HPV-18-E6, HPV-18-E7, a Her/2-neu antigen, a chimeric Her2 antigen, a Prostate Specific Antigen (PSA), bivalent PSA, ERG, Androgen receptor (AR), PAK6, Prostate Stem Cell Antigen (PSCA), NY-ESO-1, a Stratum Corneum Chymotryptic Enzyme (SCCE) antigen, Wilms tumor antigen 1 (WT-1), HIV-1 Gag, human telomerase reverse transcriptase (hTERT), Proteinase 3, Tyrosinase Related Protein 2 (TRP2), High Molecular Weight Melanoma Associated Antigen (HMW-MAA), synovial sarcoma, X (SSX)-2, carcinoembryonic antigen (CEA), Melanoma-Associated Antigen E (MAGE-A, MAGE 1, MAGE2, MAGE3, MAGE4), interleukin-13 Receptor alpha (IL13-R alpha), Carbonic anhydrase IX (CAIX), survivin, GP100, an angiogenic antigen, a ras protein, a p53 protein, a p97 melanoma antigen, KLH antigen, carcinoembryonic antigen (CEA), gp100, MART1 antigen, TRP-2, HSP-70, beta-HCG, or Testisin.
232. The process of any one of embodiments 205 and 230, wherein said tumor or cancer comprises a breast cancer or tumor, a cervical cancer or tumor, an Her2 expressing cancer or tumor, a melanoma, a pancreatic cancer or tumor, an ovarian cancer or tumor, a gastric cancer or tumor, a carcinomatous lesion of the pancreas, a pulmonary adenocarcinoma, a glioblastoma multiforme, a colorectal adenocarcinoma, a pulmonary squamous adenocarcinoma, a gastric adenocarcinoma, an ovarian surface epithelial neoplasm, an oral squamous cell carcinoma, non-mall-cell lung carcinoma, an endometrial carcinoma, a bladder cancer or tumor, a head and neck cancer or tumor, a prostate carcinoma, a renal cancer or tumor, a bone cancer or tumor, a blood cancer, or a brain cancer or tumor.
233. The process of any one of embodiments 198-232, wherein said one or more neo-epitopes comprise an infectious disease-associated-specific epitope.
234. The process of embodiment 233, wherein said infectious disease is an infectious viral disease or an infectious bacterial disease.
235. The process of embodiment 234, wherein said the infectious disease is caused by one of the following pathogens: Leishmania, Entamoeba histolytica (which causes amebiasis), Trichuris, BCG/Tuberculosis, Malaria, Plasmodium falciparum, Plasmodium malariae, Plasmodium vivax, Rotavirus, Cholera, Diptheria-Tetanus, Pertussis, Haemophilus influenzae, Hepatitis B, Human papilloma virus, Influenza seasonal), Influenza A (H1N1) Pandemic, Measles and Rubella, Mumps, Meningococcus A+C, Oral Polio Immunotherapies, mono, bi and trivalent, Pneumococcal, Rabies, Tetanus Toxoid, Yellow Fever, Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers, Arenaviruses (LCM, Junin virus, Machupo virus, Guanarito virus, Lassa Fever), Bunyaviruses (Hantaviruses, Rift Valley Fever), Flaviruses (Dengue), Filoviruses (Ebola, Marburg), Burkholderia pseudomallei, Coxiella burnetii (Q fever), Brucella species (brucellosis), Burkholderia mallei (glanders), Chlamydia psittaci (Psittacosis), Ricin toxin (from Ricinus communis), Epsilon toxin of Clostridium perfringens, Staphylococcus enterotoxin B, Typhus fever (Rickettsia prowazekii), other Rickettsias, Food- and Waterborne Pathogens, Bacteria (Diarrheagenic E. coli, Pathogenic Vibrios, Shigella species, Salmonella BCG/, Campylobacter jejuni, Yersinia enterocolitica), Viruses (Caliciviruses, Hepatitis A, West Nile Virus, LaCrosse, Calif. encephalitis, VEE, EEE, WEE, Japanese Encephalitis Virus, Kyasanur Forest Virus, Nipah virus, hantaviruses, Tickborne hemorrhagic fever viruses, Chikungunya virus, Crimean-Congo Hemorrhagic fever virus, Tickborne encephalitis viruses, Hepatitis B virus, Hepatitis C virus, Herpes Simplex virus (HSV), Human immunodeficiency virus (HIV), Human papillomavirus (HPV)), Protozoa (Cryptosporidium parvum, Cyclospora cayatanensis, Giardia lamblia, Entamoeba histolytica, Toxoplasma), Fungi (Microsporidia), Yellow fever, Tuberculosis, including drug-resistant TB, Rabies, Prions, Severe acute respiratory syndrome associated coronavirus (SARS-CoV), Coccidioides posadasii, Coccidioides immitis, Bacterial vaginosis, Chlamydia trachomatis, Cytomegalovirus, Granuloma inguinale, Hemophilus ducreyi, Neisseria gonorrhea, Treponema pallidum, Streptococcus mutans, or Trichomonas vaginalis.
236. The process of any one of embodiments 198-235, further comprising administering an adjuvant to said subject.
237. The process of embodiment 236, wherein said adjuvant comprises wherein said adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.
238. The process of any one of embodiments 198-237, further comprising administering an immune checkpoint inhibitor antagonist.
239. The process of embodiment 238, wherein said immune checkpoint inhibitor is an anti-PD-L1/PD-L2 antibody or fragment thereof, an anti-PD-1 antibody or fragment thereof, an anti-CTLA-4 antibody or fragment thereof, or an anti-B7-H4 antibody or fragment thereof.
240. The process of any one of embodiments 198-239, wherein said administering generates a personalized enhanced anti-disease, or anti-condition immune response in said subject.
241. The process of embodiment 240, wherein said immune response comprises an anti-cancer or anti-tumor response.
242. The process of embodiment 240, wherein said immune response comprises an anti-infectious disease response.
243. The process of embodiment 242, wherein said infectious disease comprises a viral infection or bacterial infection.
244. The process of any one of embodiments 198-243, wherein said process allows personalized treatment or prevention of said disease or condition in said subject.
245. The process of any one of embodiments 198-244, wherein said personalized immunotherapy increases survival time in said subject having said disease or condition.
246. A viral-like particle produced by the process of any one of embodiments 198-214, 218, and 220-235.
247. A vaccinia virus strain produced by the process of any one of embodiments 198-214, 218, and 220-235.
248. A DNA immunotherapy produced by the process of any one of embodiments 198-212, 215-218, and 220-235.
249. A peptide immunotherapy produced by the process of any one of embodiments 198-212, 219, and 220-235.
250. A pharmaceutical composition comprising the Listeria of embodiment 197.
251. A pharmaceutical composition comprising the viral-like particle of embodiment 246.
252. A pharmaceutical composition comprising the vaccinia virus strain of embodiment 247.
253. A pharmaceutical composition comprising the DNA immunotherapy of embodiment 248.
254. A pharmaceutical composition comprising the peptide immunotherapy of embodiment 249.
255. A system for creating personalized immunotherapy for a subject, comprising: at least one processor and at least one storage medium containing program instructions for execution by the processor, the program instructions causing the processor to execute steps comprising:
(a) receiving output data containing all neo-epitopes and the human leukocyte antigen (HLA) type of the subject;
(b) scoring the hydrophobicity of each neo-epitope and removing epitopes that score above a certain threshold;
(c) numerically rating the remaining neo-epitopes based on their ability to bind to subject HLA and on their predictive MHC binding scores;
(d) inserting the amino acid sequence of each neo-epitope into a plasmid;
(e) scoring the hydrophobicity of each construct and removing any constructs that score above a certain threshold;
(f) reverse translating the amino acid sequence of each construct into the corresponding DNA sequence, starting with the highest scored construct;
(g) inserting additional neo-epitopes into the plasmid construct in order of ranking until a predetermined upper limit is reached;
(h) adding a DNA sequence tag to the end of the construct in order to measure the immunotherapeutic response in the subject; and
(i) optimizing the DNA sequence encoding the neo-epitopes and the DNA sequence tag for expression and secretion in Listeria monocytogenes.
   256. The system of embodiment 255, wherein the preferred output data is in FASTA format.
   257. The system of any embodiment 255-256, wherein the hydrophobicity is scaled using the Kyte-Doolittle hydropathy plot.
   258. The system of any embodiment 255-257, wherein all neo-epitopes scoring above a 1.6 on the Kyte-Doolittle plot are removed or de-selected.
   259. The system of any embodiment 255-258, wherein each neo-epitope's ability to bind to subject HLA is rated using the Immune Epitope Database (IED).
   260. The system of any embodiment 255-259, wherein each neo-epitope is 21 amino acids in size (21 mer).
   261. The system of any embodiment 255-260, wherein the DNA tag is linked to the neo-epitopes via a linker.
   262. The system of any embodiment 255-261, wherein the linker is a 4× glycine linker.
   263. The system of any embodiment 255-262, wherein the DNA sequence tag of step (h) is SIINFEKL-6×His.
   264. The system of any embodiment 255-263, wherein neo-epitopes known to have immunosuppressive properties are removed from consideration before step (a).

In the following examples, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### EXAMPLES

### Materials and Experimental Methods (Examples 1-2)

### Cell Lines

The C57BL/6 syngeneic TC-1 tumor was immortalized with HPV-16 E6 and E7 and transformed with the c-Ha-ras oncogene. TC-1, provided by T. C. Wu (Johns Hopkins University School of Medicine, Baltimore, Md.) is a highly tumorigenic lung epithelial cell expressing low levels of with HPV-16 E6 and E7 and transformed with the c-Ha-ras oncogene. TC-1 was grown in RPMI 1640, 10% FCS, 2 mM L-glutamine, 100 U/ml penicillin, 100 μg/ml streptomycin, 100 μM nonessential amino acids, 1 mM sodium pyruvate, 50 micromolar (mcM) 2-ME, 400 microgram (mcg)/ml G418, and 10% National Collection Type Culture-109 medium at 37° with 10% CO₂. C3 is a mouse embryo cell from C57BL/6 mice immortalized with the complete genome of HPV 16 and transformed with pEJ-ras. EL-4/E7 is the thymoma EL-4 retrovirally transduced with E7.

L. monocytogenes Strains and Propagation

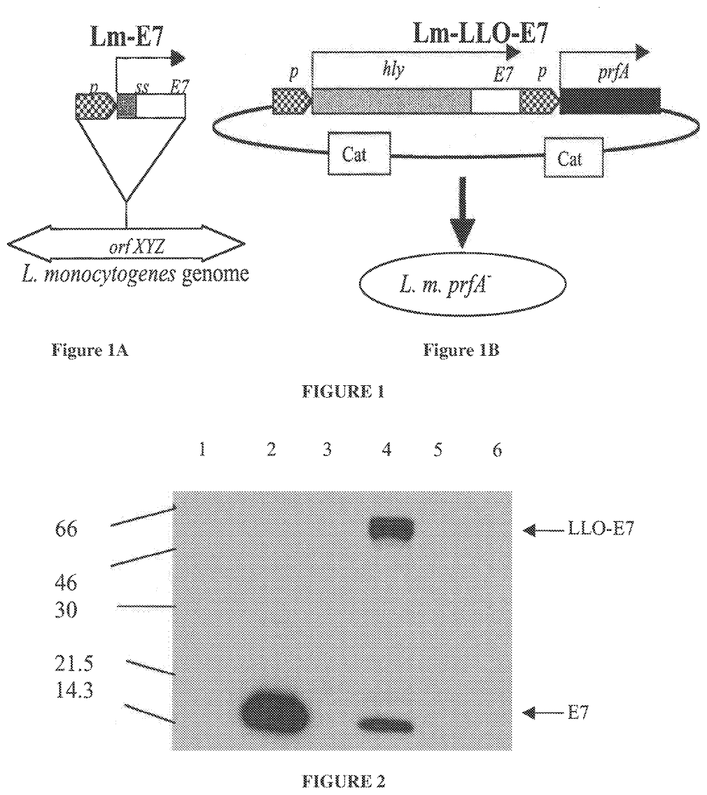

Listeria strains used were Lm-LLO-E7, also referred to herein as ADXS11-001, (hly-E7 fusion gene in an episomal expression system; FIG. 1A), Lm-E7 (single-copy E7 gene cassette integrated into Listeria genome), Lm-LLO-NP (“DP-L2028”; hly-NP fusion gene in an episomal expression system), and Lm-Gag (“ZY-18”; single-copy HIV-1 Gag gene cassette integrated into the chromosome). E7 was amplified by PCR using the primers 5′-GGCTCGAGCATGGAGATACACC-3′ (SEQ ID No: 24; XhoI site is underlined) and 5′-GGGGACTAGTTTATGGTTTCTGAGAACA-3′ (SEQ ID No: 25; SpeI site is underlined) and ligated into pCR2.1 (Invitrogen, San Diego, Calif.). E7 was excised from pCR2.1 by XhoI/SpeI digestion and ligated into pGG-55. The hly-E7 fusion gene and the pluripotential transcription factor prfA were cloned into pAM401, a multicopy shuttle plasmid (Wirth R et al, J Bacteriol, 165: 831, 1986), generating pGG-55. The hly promoter drives the expression of the first 441 AA of the hly gene product, (lacking the hemolytic C-terminus, referred to below as “ΔLLO,” and having the sequence set forth in SEQ ID No: 3), which is joined by the XhoI site to the E7 gene, yielding a hly-E7 fusion gene that is transcribed and secreted as LLO-E7. Transformation of a prfA negative strain of Listeria, XFL-7 (provided by Dr. Hao Shen, University of Pennsylvania), with pGG-55 selected for the retention of the plasmid in vivo (FIGS. 1A-B). The hly promoter and gene fragment were generated using primers 5′-GGGGGCTAGCCCTCCTTTGATTAGTATATTC-3′ (SEQ ID No: 26; NheI site is underlined) and 5′-CTCCCTCGAGATCATAATTTACTTCATC-3′ (SEQ ID No: 27; XhoI site is underlined). The prfA gene was PCR amplified using primers 5′-GACTACAAGGACGATGACCGACAAGTGATAACCCGGGATCTAAATAAATCCGTT T-3′ (SEQ ID No: 28; XbaI site is underlined) and 5′-CCCGTCGACCAGCTCTTCTTGGTGAAG-3′ (SEQ ID No: 29; SalI site is underlined). Lm-E7 was generated by introducing an expression cassette containing the hly promoter and signal sequence driving the expression and secretion of E7 into the orfZ domain of the LM genome. E7 was amplified by PCR using the primers 5′-GCGGATCCCATGGAGATACACCTAC-3′ (SEQ ID No: 30; BamHI site is underlined) and 5′-GCTCTAGATTATGGTTTCTGAG-3′ (SEQ ID No: 31; XbaI site is underlined). E7 was then ligated into the pZY-21 shuttle vector. LM strain 10403S was transformed with the resulting plasmid, pZY-21-E7, which includes an expression cassette inserted in the middle of a 1.6-kb sequence that corresponds to the orfX, Y, Z domain of the LM genome. The homology domain allows for insertion of the E7 gene cassette into the orfZ domain by homologous recombination. Clones were screened for integration of the E7 gene cassette into the orfZ domain. Bacteria were grown in brain heart infusion medium with (Lm-LLO-E7 and Lm-LLO-NP) or without (Lm-E7 and ZY-18) chloramphenicol (20 μg/ml). Bacteria were frozen in aliquots at −80° C. Expression was verified by Western blotting (FIG. 2).

### Western Blotting

Listeria strains were grown in Luria-Bertoni medium at 37° C. and were harvested at the same optical density measured at 600 nm. The supernatants were TCA precipitated and resuspended in 1× sample buffer supplemented with 0.1 N NaOH. Identical amounts of each cell pellet or each TCA-precipitated supernatant were loaded on 4-20% Tris-glycine SDS-PAGE gels (NOVEX, San Diego, Calif.). The gels were transferred to polyvinylidene difluoride and probed with an anti-E7 monoclonal antibody (mAb) (Zymed Laboratories, South San Francisco, Calif.), then incubated with HRP-conjugated anti-mouse secondary Ab (Amersham Pharmacia Biotech, Little Chalfont, U.K.), developed with Amersham ECL detection reagents, and exposed to Hyperfilm (Amersham Pharmacia Biotech).

### Measurement of Tumor Growth

Tumors were measured every other day with calipers spanning the shortest and longest surface diameters. The mean of these two measurements was plotted as the mean tumor diameter in millimeters against various time points. Mice were sacrificed when the tumor diameter reached 20 mm. Tumor measurements for each time point are shown only for surviving mice.

### Effects of Listeria Recombinants on Established Tumor Growth

Six- to 8-wk-old C57BL/6 mice (Charles River) received 2×10⁵ TC-1 cells s.c. on the left flank. One week following tumor inoculation, the tumors had reached a palpable size of 4-5 mm in diameter. Groups of eight mice were then treated with 0.1 LD₅₀ i.p. Lm-LLO-E7 (10⁷ CFU), Lm-E7 (10⁶ CFU), Lm-LLO-NP (10⁷ CFU), or Lm-Gag (5×10⁵ CFU) on days 7 and 14.

### ⁵¹Cr Release Assay

C57BL/6 mice, 6-8 wk old, were immunized i.p. with 0.1LD₅₀ Lm-LLO-E7, Lm-E7, Lm-LLO-NP, or Lm-Gag. Ten days post-immunization, spleens were harvested. Splenocytes were established in culture with irradiated TC-1 cells (100:1, splenocytes:TC-1) as feeder cells; stimulated in vitro for 5 days, then used in a standard ⁵¹Cr release assay, using the following targets: EL-4, EL-4/E7, or EL-4 pulsed with E7 H-2b peptide (RAHYNIVTF). E:T cell ratios, performed in triplicate, were 80:1, 40:1, 20:1, 10:1, 5:1, and 2.5:1. Following a 4-h incubation at 37° C., cells were pelleted, and 50 μl supernatant was removed from each well. Samples were assayed with a Wallac 1450 scintillation counter (Gaithersburg, Md.). The percent specific lysis was determined as [(experimental counts per minute (cpm)−spontaneous cpm)/(total cpm−spontaneous cpm)]×100.

### TC-1-Specific Proliferation

C57BL/6 mice were immunized with 0.1 LD₅₀ and boosted by i.p. injection 20 days later with 1 LD₅₀ Lm-LLO-E7, Lm-E7, Lm-LLO-NP, or Lm-Gag. Six days after boosting, spleens were harvested from immunized and naive mice. Splenocytes were established in culture at 5×10⁵/well in flat-bottom 96-well plates with 2.5×10⁴, 1.25×10⁴, 6×10³, or 3×10³ irradiated TC-1 cells/well as a source of E7 Ag, or without TC-1 cells or with 10 μg/ml Con A. Cells were pulsed 45 h later with 0.5 μCi [³H]thymidine/well. Plates were harvested 18 h later using a Tomtec harvester 96 (Orange, Conn.), and proliferation was assessed with a Wallac 1450 scintillation counter. The change in cpm was calculated as experimental cpm−no Ag cpm.

### Flow Cytometric Analysis

C57BL/6 mice were immunized intravenously (i.v.) with 0.1 LD₅₀ Lm-LLO-E7 or Lm-E7 and boosted 30 days later. Three-color flow cytometry for CD8 (53-6.7, PE conjugated), CD62 ligand (CD62L; MEL-14, APC conjugated), and E7 H-2Db tetramer was performed using a FACSCalibur® flow cytometer with CellQuest® software (Becton Dickinson, Mountain View, Calif.). Splenocytes harvested 5 days after the boost were stained at room temperature (rt) with H-2Db tetramers loaded with the E7 peptide (RAHYNIVTF) or a control (HIV-Gag) peptide. Tetramers were used at a 1/200 dilution and were provided by Dr. Larry R. Pease (Mayo Clinic, Rochester, Minn.) and by the NIAID Tetramer Core Facility and the NIH AIDS Research and Reference Reagent Program. Tetramer⁺, CD8⁺, CD62L^{low} cells were analyzed.

### B16F0-Ova Experiment

24 C57BL/6 mice were inoculated with 5×10⁵ B16F0-Ova cells. On days 3, 10 and 17, groups of 8 mice were immunized with 0.1 LD₅₀ Lm-OVA (10⁶ cfu), Lm-LLO-OVA (10⁸ cfu) and eight animals were left untreated.

### Statistics

For comparisons of tumor diameters, mean and SD of tumor size for each group were determined, and statistical significance was determined by Student's t test. p<0.05 was considered significant.

### Example 1: LLO-Antigen Fusions Induce Anti-Tumor Immunity

### Results

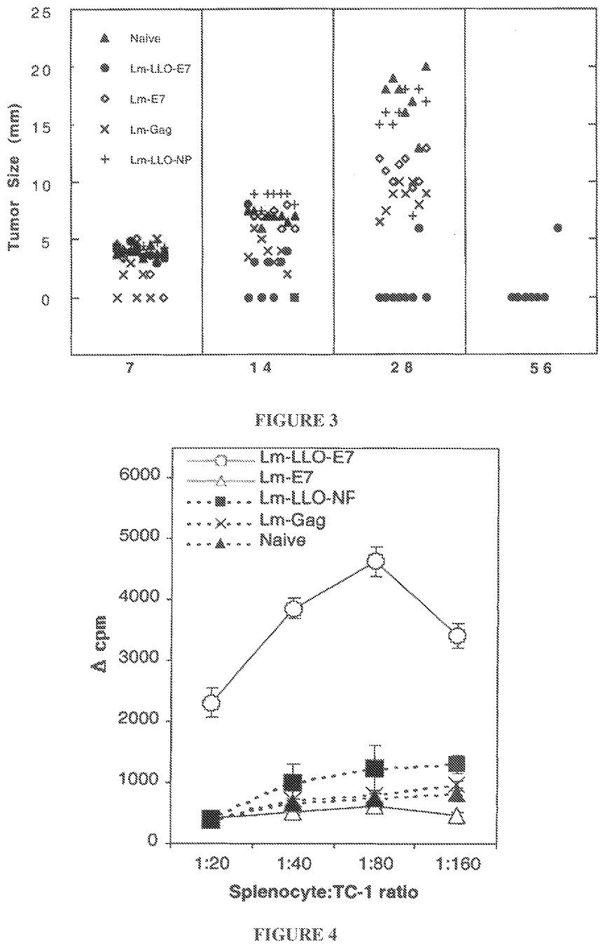

Lm-E7 and Lm-LLO-E7 were compared for their abilities to impact on TC-1 growth. Subcutaneous tumors were established on the left flank of C57BL/6 mice. Seven days later tumors had reached a palpable size (4-5 mm). Mice were vaccinated on days 7 and 14 with 0.1 LD₅₀ Lm-E7, Lm-LLO-E7, or, as controls, Lm-Gag and Lm-LLO-NP. Lm-LLO-E7 induced complete regression of 75% of established TC-1 tumors, while tumor growth was controlled in the other 2 mice in the group (FIG. 3). By contrast, immunization with Lm-E7 and Lm-Gag did not induce tumor regression. This experiment was repeated multiple times, always with very similar results. In addition, similar results were achieved for Lm-LLO-E7 under different immunization protocols. In another experiment, a single immunization was able to cure mice of established 5 mm TC-1 tumors.

In other experiments, similar results were obtained with 2 other E7-expressing tumor cell lines: C3 and EL-4/E7. To confirm the efficacy of vaccination with Lm-LLO-E7, animals that had eliminated their tumors were re-challenged with TC-1 or EL-4/E7 tumor cells on day 60 or day 40, respectively. Animals immunized with Lm-LLO-E7 remained tumor free until termination of the experiment (day 124 in the case of TC-1 and day 54 for EL-4/E7).

Thus, expression of an antigen as a fusion protein with ALLO enhances the immunogenicity of the antigen.

### Example 2: LM-LLO-E7 Treatment Elicits TC-1 Specific Splenocyte Proliferation

To measure induction of T cells by Lm-E7 with Lm-LLO-E7, TC-1-specific proliferative responses, a measure of antigen-specific immunocompetence, were measured in immunized mice. Splenocytes from Lm-LLO-E7-immunized mice proliferated when exposed to irradiated TC-1 cells as a source of E7, at splenocyte: TC-1 ratios of 20:1, 40:1, 80:1, and 160:1 (FIG. 4). Conversely, splenocytes from Lm-E7 and rLm control-immunized mice exhibited only background levels of proliferation.

### Example 3: ActA-E7 and PEST-E7 Fusions Confer Anti-Tumor Immunity

### Materials and Methods

### Construction of Lm-ActA-E7

Lm-ActA-E7 is a recombinant strain of LM, comprising a plasmid that expresses the E7 protein fused to a truncated version of the actA protein. Lm-actA-E7 was generated by introducing a plasmid vector pDD-1, constructed by modifying pDP-2028, into Listeria. pDD-1 comprises an expression cassette expressing a copy of the 310 bp hly promoter and the hly signal sequence (ss), which drives the expression and secretion of ActA-E7; 1170 bp of the actA gene that comprises four PEST sequences (SEQ ID NO: 19) (the truncated ActA polypeptide consists of the first 390 AA of the molecule, SEQ ID NO: 11); the 300 bp HPV E7 gene; the 1019 bp prfA gene (controls expression of the virulence genes); and the CAT gene (chloramphenicol resistance gene) for selection of transformed bacteria clones (Sewell et al. (2004), Arch. Otolaryngol. Head Neck Surg., 130: 92-97).

The hly promoter (pHly) and gene fragment were PCR amplified from pGG55 (Example 1) using primer 5′-GGGGTCTAGACCTCCTTTGATTAGTATATTC-3′ (Xba I site is underlined; SEQ ID NO: 32) and primer 5′-ATCTTCGCTATCTGTCGCCGCGGCGCGTGCTTCAGTTTGTTGCGC-′3 (Not I site is underlined. The first 18 nucleotides are the ActA gene overlap; SEQ ID NO: 33). The actA gene was PCR amplified from the LM 10403s wild type genome using primer 5′-GCGCAACAAACTGAAGCAGCGGCCGCGGCGACAGATAGCGAAGAT-3′ (NotI site is underlined; SEQ ID NO: 34) and primer 5′-TGTAGGTGTATCTCCATGCTCGAGAGCTAGGCGATCAATTTC-3′ (XhoI site is underlined; SEQ ID NO: 35). The E7 gene was PCR amplified from pGG55 (pLLO-E7) using primer 5′-GGAATTGATCGCCTAGCTCTCGAGCATGGAGATACACCTACA-3′ (XhoI site is underlined; SEQ ID NO: 36) and primer 5′-AAACGGATTTATTTAGATCCCGGGTTATGGTTTCTGAGAACA-3′ (XmaI site is underlined; SEQ ID NO: 37). The prfA gene was PCR amplified from the LM 10403s wild-type genome using primer 5′-TGTTCTCAGAAACCATAACCCGGGATCTAAATAAATCCGTTT-3′ (XmaI site is underlined; SEQ ID NO: 38) and primer 5′-GGGGGTCGACCAGCTCTTCTTGGTGAAG-3′ (SalI site is underlined; SEQ ID NO: 39). The hly promoter-actA gene fusion (pHly-actA) was PCR generated and amplified from purified pHly DNA and purified actA DNA using the upstream pHly primer (SEQ ID NO: 32) and downstream actA primer (SEQ ID NO: 35).

The E7 gene fused to the prfA gene (E7-prfA) was PCR generated and amplified from purified E7 DNA and purified prfA DNA using the upstream E7 primer (SEQ ID NO: 36) and downstream prfA gene primer (SEQ ID NO: 39).

The pHly-actA fusion product fused to the E7-prfA fusion product was PCR generated and amplified from purified fused pHly-actA DNA product and purified fused E7-prfA DNA product using the upstream pHly primer (SEQ ID NO: 32) and downstream prfA gene primer (SEQ ID NO: 39) and ligated into pCRII (Invitrogen, La Jolla, Calif.). Competent E. coli (TOP10′F, Invitrogen, La Jolla, Calif.) were transformed with pCRII-ActAE7. After lysis and isolation, the plasmid was screened by restriction analysis using BamHI (expected fragment sizes 770 bp and 6400 bp (or when the insert was reversed into the vector: 2500 bp and 4100 bp)) and BstXI (expected fragment sizes 2800 bp and 3900 bp) and also screened with PCR analysis using the upstream pHly primer (SEQ ID NO: 32) and the downstream prfA gene primer (SEQ ID NO: 39).

The pHly-actA-E7-prfA DNA insert was excised from pCRII by double digestion with Xba I and Sal I and ligated into pDP-2028 also digested with Xba I and Sal I. After transforming TOP10′F competent E. coli (Invitrogen, La Jolla, Calif.) with expression system pActAE7, chloramphenicol resistant clones were screened by PCR analysis using the upstream pHly primer (SEQ ID NO: 32) and the downstream PrfA gene primer (SEQ ID NO: 39). A clone comprising pActAE7 was grown in brain heart infusion medium (with chloramphenicol (20 mcg (microgram)/ml (milliliter), Difco, Detroit, Mich.) and pActAE7 was isolated from the bacteria cell using a midiprep DNA purification system kit (Promega, Madison, Wis.). A prfA-negative strain of penicillin-treated Listeria (strain XFL-7) was transformed with expression system pActAE7, as described in Ikonomidis et al. (1994, J. Exp. Med. 180: 2209-2218) and clones were selected for the retention of the plasmid in vivo. Clones were grown in brain heart infusion with chloramphenicol (20 mcg/ml) at 37° C. Bacteria were frozen in aliquots at −80° C.

### Immunoblot Verification of Antigen Expression

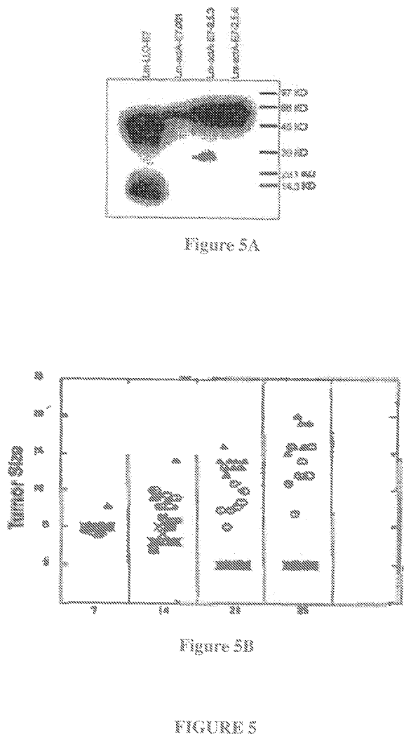

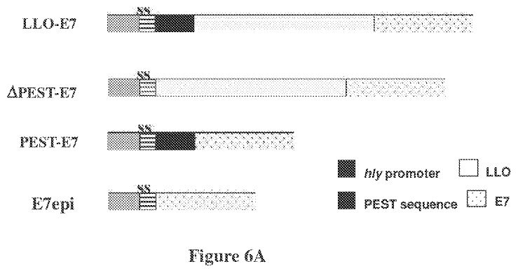

To verify that Lm-ActA-E7 secretes ActA-E7, (about 64 kD), Listeria strains were grown in Luria-Bertoni (LB) medium at 37° C. Protein was precipitated from the culture supernatant with trichloroacetic acid (TCA) and resuspended in 1× sample buffer with 0.1N sodium hydroxide. Identical amounts of each TCA precipitated supernatant were loaded on 4% to 20% Tris-glycine sodium dodecyl sulfate-polyacrylamide gels (NOVEX, San Diego, Calif.). Gels were transferred to polyvinylidene difluoride membranes and probed with 1:2500 anti-E7 monoclonal antibody (Zymed Laboratories, South San Francisco, Calif.), then with 1:5000 horseradish peroxidase-conjugated anti-mouse IgG (Amersham Pharmacia Biotech, Little Chalfont, England). Blots were developed with Amersham enhanced chemiluminescence detection reagents and exposed to autoradiography film (Amersham) (FIG. 5A).

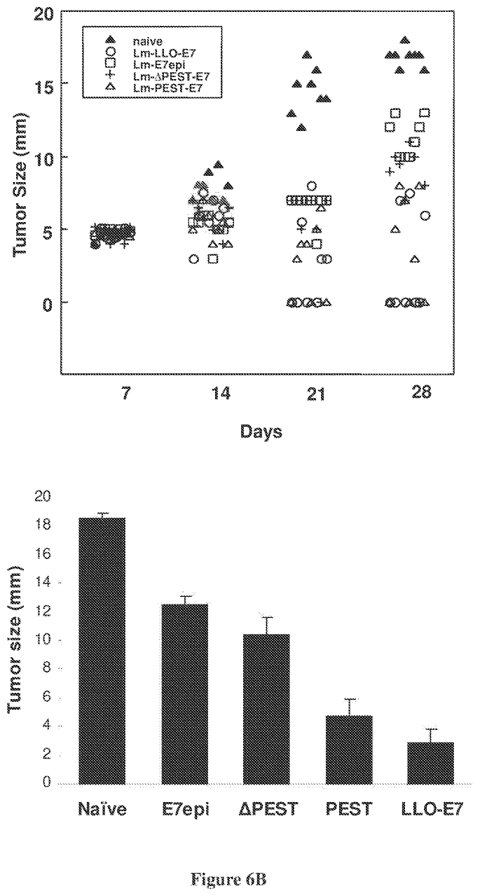

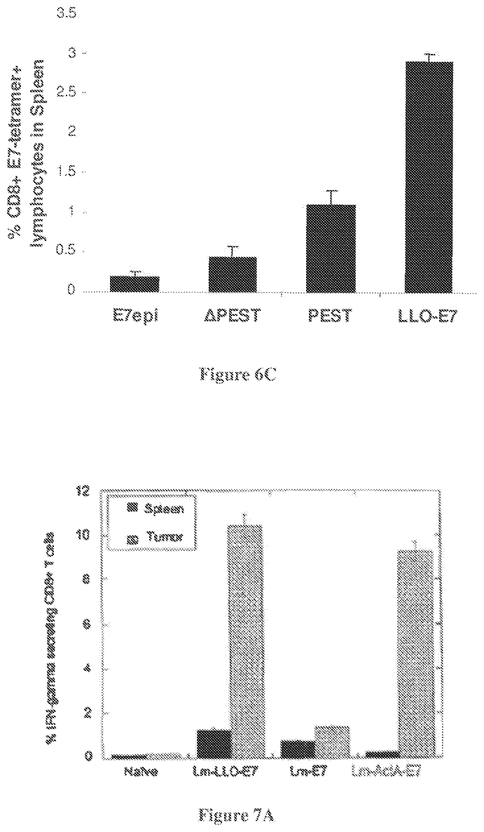

### Construction of Lm-PEST-E7, Lm-ΔPEST-E7, and Lm-E7epi (FIG. 6A)

Lm-PEST-E7 is identical to Lm-LLO-E7, except that it contains only the promoter and PEST sequence of the hly gene, specifically the first 50 AA of LLO. To construct Lm-PEST-E7, the hly promoter and PEST regions were fused to the full-length E7 gene using the SOE (gene splicing by overlap extension) PCR technique. The E7 gene and the hly-PEST gene fragment were amplified from the plasmid pGG-55, which contains the first 441 AA of LLO, and spliced together by conventional PCR techniques. To create a final plasmid, pVS16.5, the hly-PEST-E7 fragment and the prfA gene were subcloned into the plasmid pAM401, which includes a chloramphenicol resistance gene for selection in vitro, and the resultant plasmid was used to transform XFL-7.

Lm-ΔPEST-E7 is a recombinant Listeria strain that is identical to Lm-LLO-E7 except that it lacks the PEST sequence. It was made essentially as described for Lm-PEST-E7, except that the episomal expression system was constructed using primers designed to remove the PEST-containing region (bp 333-387) from the hly-E7 fusion gene. Lm-E7epi is a recombinant strain that secretes E7 without the PEST region or LLO. The plasmid used to transform this strain contains a gene fragment of the hly promoter and signal sequence fused to the E7 gene. This construct differs from the original Lm-E7, which expressed a single copy of the E7 gene integrated into the chromosome. Lm-E7epi is completely isogenic to Lm-LLO-E7, Lm-PEST-E7, and Lm-ΔPEST-E7 except for the form of the E7 antigen expressed.

### Results

To compare the anti-tumor immunity induced by Lm-ActA-E7 versus Lm-LLO-E7, 2×10⁵ TC-1 tumor cells were implanted subcutaneously in mice and allowed to grow to a palpable size (approximately 5 millimeters [mm]). Mice were immunized i.p. with one LD₅₀ of either Lm-ActA-E7 (5×10⁸ CFU), (crosses) Lm-LLO-E7 (10⁸ CFU) (squares) or Lm-E7 (10⁶ CFU) (circles) on days 7 and 14. By day 26, all of the animals in the Lm-LLO-E7 and Lm-ActA-E7 were tumor free and remained so, whereas all of the naive animals (triangles) and the animals immunized with Lm-E7 grew large tumors (FIG. 5B). Thus, vaccination with ActA-E7 fusions causes tumor regression.

In addition, Lm-LLO-E7, Lm-PEST-E7, Lm-ΔPEST-E7, and Lm-E7epi were compared for their ability to cause regression of E7-expressing tumors. s.c. TC-1 tumors were established on the left flank of 40 C57BL/6 mice. After tumors had reached 4-5 mm, mice were divided into 5 groups of 8 mice. Each groups was treated with 1 of 4 recombinant LM immunotherapies, and 1 group was left untreated. Lm-LLO-E7 and Lm-PEST-E7 induced regression of established tumors in 5/8 and 3/8 cases, respectively. There was no statistical difference between the average tumor size of mice treated with Lm-PEST-E7 or Lm-LLO-E7 at any time point. However, the immunotherapies that expressed E7 without the PEST sequences, Lm-ΔPEST-E7 and Lm-E7epi, failed to cause tumor regression in all mice except one (FIG. 6B, top panel). This was representative of 2 experiments, wherein a statistically significant difference in mean tumor sizes at day 28 was observed between tumors treated with Lm-LLO-E7 or Lm-PEST-E7 and those treated with Lm-E7epi or Lm-ΔPEST-E7; P<0.001, Student's t test; FIG. 6B, bottom panel). In addition, increased percentages of tetramer-positive splenocytes were seen reproducibly over 3 experiments in the spleens of mice vaccinated with PEST-containing immunotherapies (FIG. 6C). Thus, vaccination with PEST-E7 fusions causes tumor regression.

### Example 4: Fusion of E7 to LLO, Acta, or A Pest-Like Sequence Enhances E7-Specific Immunity and Generates Tumor-Infiltrating E7-Specific CD8⁺ Cells

### Materials and Experimental Methods

500 mcl (microliter) of MATRIGEL®, comprising 100 mcl of 2×10⁵ TC-1 tumor cells in phosphate buffered saline (PBS) plus 400 mcl of MATRIGEL® (BD Biosciences, Franklin Lakes, N.J.) were implanted subcutaneously on the left flank of 12 C57BL/6 mice (n=3). Mice were immunized intraperitoneally on day 7, 14 and 21, and spleens and tumors were harvested on day 28. Tumor MATRIGELs were removed from the mice and incubated at 4° C. overnight in tubes containing 2 milliliters (ml) of RP 10 medium on ice. Tumors were minced with forceps, cut into 2 mm blocks, and incubated at 37° C. for 1 hour with 3 ml of enzyme mixture (0.2 mg/ml collagenase-P, 1 mg/ml DNAse-1 in PBS). The tissue suspension was filtered through nylon mesh and washed with 5% fetal bovine serum+0.05% of NaN₃ in PBS for tetramer and IFN-gamma staining.

Splenocytes and tumor cells were incubated with 1 micromole (mcm) E7 peptide for 5 hours in the presence of brefeldin A at 10⁷ cells/ml. Cells were washed twice and incubated in 50 mcl of anti-mouse Fc receptor supernatant (2.4 G2) for 1 hour or overnight at 4° C. Cells were stained for surface molecules CD8 and CD62L, permeabilized, fixed using the permeabilization kit Golgi-stop® or Golgi-Plug® (Pharmingen, San Diego, Calif.), and stained for IFN-gamma. 500,000 events were acquired using two-laser flow cytometer FACSCalibur and analyzed using Cellquest Software (Becton Dickinson, Franklin Lakes, N.J.). Percentages of IFN-gamma secreting cells within the activated (CD62L^{low}) CD8⁺ T cells were calculated.

For tetramer staining, H-2D^{b} tetramer was loaded with phycoerythrin (PE)-conjugated E7 peptide (RAHYNIVTF, SEQ ID NO: 40), stained at rt for 1 hour, and stained with anti-allophycocyanin (APC) conjugated MEL-14 (CD62L) and FITC-conjugated CD8⁺ at 4° C. for 30 min Cells were analyzed comparing tetramer⁺CD8⁺ CD62L^{low} cells in the spleen and in the tumor.

### Results

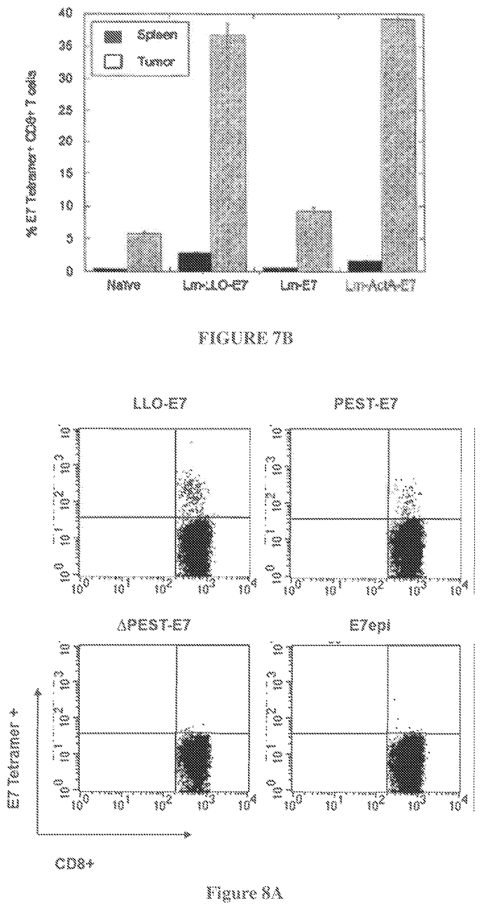

To analyze the ability of Lm-ActA-E7 to enhance antigen specific immunity, mice were implanted with TC-1 tumor cells and immunized with either Lm-LLO-E7 (1×10⁷ CFU), Lm-E7 (1×10⁶ CFU), or Lm-ActA-E7 (2×10⁸ CFU), or were untreated (naïve). Tumors of mice from the Lm-LLO-E7 and Lm-ActA-E7 groups contained a higher percentage of IFN-gamma-secreting CD8⁺ T cells (FIG. 7A) and tetramer-specific CD8⁺ cells (FIG. 7B) than in Lm-E7 or naive mice.

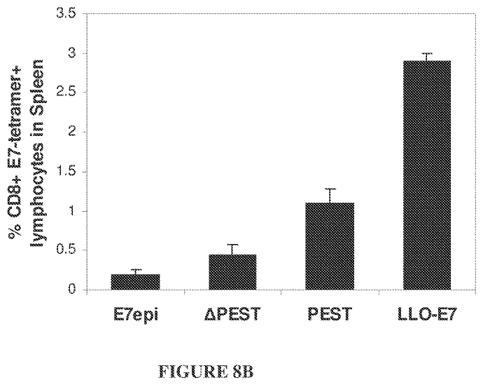

In another experiment, tumor-bearing mice were administered Lm-LLO-E7, Lm-PEST-E7, Lm-ΔPEST-E7, or Lm-E7epi, and levels of E7-specific lymphocytes within the tumor were measured. Mice were treated on days 7 and 14 with 0.1 LD₅₀ of the 4 immunotherapies. Tumors were harvested on day 21 and stained with antibodies to CD62L, CD8, and with the E7/Db tetramer. An increased percentage of tetramer-positive lymphocytes within the tumor were seen in mice vaccinated with Lm-LLO-E7 and Lm-PEST-E7 (FIG. 8A). This result was reproducible over three experiments (FIG. 8B).

Thus, Lm-LLO-E7, Lm-ActA-E7, and Lm-PEST-E7 are each efficacious at induction of tumor-infiltrating CD8⁺ T cells and tumor regression.

### Example 5: LLO and ActA Fusions Reduce Autochthonous (Spontaneous) Tumors in E6/E7 Transgenic Mice

To determine the impact of the Lm-LLO-E7 and Lm-ActA-E7 immunotherapies on autochthonous tumors in the E6/E7 transgenic mouse, 6 to 8 week old mice were immunized with 1×10⁸ Lm-LLO-E7 or 2.5×10⁸ Lm-ActA-E7 once per month for 8 months. Mice were sacrificed 20 days after the last immunization and their thyroids removed and weighed. This experiment was performed twice (Table 1).

TABLE 1
Thyroid weight (mg) in unvaccinated and vaccinated transgenic mice at 8
months of age (mg)*.

| | | Lm-LLO- | | Lm-LLO- | | Lm-ActA- | |
|---|---|---|---|---|---|---|---|
| Untreated | ±S.D. | NP | ±S.D. | E7 | ±S.D. | E7 | ±S.D. |
| Expt. 1 | 123 | 385 | 130 | 225 | 54 | 305 | 92 |
| 408 | | | | | | | |
| Expt. 2 | 94 | 503 | 86 | 239 | 68 | 275 | 84 |
| 588 | | | | | | | |

*Statistical analyses performed using Student's t test showed that the difference in thyroid weight between Lm-LLO-NP treated mice and untreated mice was not significant but that the difference between Lm-LLO-E7 and Lm-ActA-E7 treated mice was highly significant (p < 0.001)

The difference in thyroid weight between Lm-LLO-E7 treated mice and untreated mice and between Lm-LLO-ActA treated mice and untreated mice was significant (p<0.001 and p<0.05, respectively) for both experiments, while the difference between Lm-LLO-NP treated mice (irrelevant antigen control) and untreated mice was not significant (Student's t test), showing that Lm-LLO-E7 and Lm-ActA-E7 controlled spontaneous tumor growth. Thus, immunotherapies of the present invention prevent formation of new E7-expressing tumors.

To summarize the findings in the above Examples, LLO-antigen and ActA-antigen fusions (a) induce tumor-specific immune response that include tumor-infiltrating antigen-specific T cells; and are capable of inducing tumor regression and controlling tumor growth of both normal and particularly aggressive tumors; (b) overcome tolerance to self antigens; and (c) prevent spontaneous tumor growth. These findings are generalizable to a large number of antigens, PEST-like sequences, and tumor types, as evidenced by their successful implementation with a variety of different antigens, PEST-like sequences, and tumor types.

### Example 6: LM-LLO-E7 Immunotherapies are Safe and Improve Clinical Indicators in Cervical Cancer Patients

### Materials and Experimental Methods

Inclusion Criteria.

All patients in the trial were diagnosed with “advanced, progressive or recurrent cervical cancer,” and an assessment at the time of entry indicated that all were staged as having IVB disease. All patients manifested a positive immune response to an anergy panel containing 3 memory antigens selected from candidin, mumps, tetanus, or Tuberculin Purified Protein Derivative (PPD); were not pregnant or HIV positive, had taken no investigational drugs within 4 weeks, and were not receiving steroids.

Protocol:

Patients were administered 2 vaccinations at a 3-week interval as a 30-minute intravenous (IV) infusion in 250 ml of normal saline to inpatients. After 5 days, patients received a single course of IV ampicillin and were released with an additional 10 days of oral ampicillin Karnofsky Performance Index, which is a measurement of overall vitality and quality of life such as appetite, ability to complete daily tasks, restful sleep, etc, was used to determine overall well-being. In addition, the following indicators of safety and general wellbeing were determined: alkaline phosphatase; bilirubin, both direct and total; gamma glutamyl transpeptidase (ggt); cholesterol; systole, diastole, and heart rate; Eastern Collaborative Oncology Group's (ECOG)'s criteria for assessing disease progression—a Karnofsky like—quality of life indicator; hematocrit; hemoglobin; platelet levels; lymphocytes levels; AST (aspartate aminotransferase); ALT (alanine aminotransferase); and LDH (lactate dehydrogenase). Patients were followed at 3 weeks and 3 months subsequent to the second dosing, at which time Response Evaluation Criteria in Solid Tumors (RECIST) scores of the patients were determined, scans were performed to determine tumor size, and blood samples were collected for immunological analysis at the end of the trial, which includes the evaluation of IFN-γ, IL-4, CD4+ and CD8⁺ cell populations.

Listeria Strains:

The creation of LM-LLO-E7 is described in Example 1.

### Results

Prior to the clinical trial, a preclinical experiment was performed to determine the anti-tumor efficacy of intravenous (i.v.) vs. i.p. administration of LM-LLO-E7. A tumor containing 1×10⁴ TC-1 cells was established sub-cutaneously. On days 7 and 14, mice were immunized with either 10⁸ LM-LLO-E7 i.p. or LM-LLO-E7 i.v. at doses of 10⁸, 10⁷, 10⁶, or 10⁵. At day 35, 5/8 of the mice that received 10⁸ LM-LLO-E7 by either route or 10⁷ LM-LLO-E7 i.v, and 4/8 of the mice that received 10⁶ LM-LLO-E7 i.v, were cured. By contrast, doses of less than 10⁷ or in some cases even 10⁸ LM-LLO-E7 administered i.p. were ineffective at controlling tumor growth. Thus, i.v. administration of LM-LLO-E7 is more effective than i.p. administration.

### Clinical Trial

A phase I/II clinical trial was conducted to assess safety and efficacy of LM-LLO-E7 immunotherapies in patients with advanced, progressive, or recurrent cervical cancer. 5 patients each were assigned to cohorts 1-2, which received 1×10⁹ or 3.3×10⁹ CFU, respectfully. An additional 5 patients each will be assigned to cohorts 3-4, which will receive 1×10¹⁰ or 3.31×10¹⁰ CFU, respectfully.

### Safety Data

### First Cohort

All patients in the first cohort reported onset of mild-to-moderate fever and chills within 1-2 hours after onset of the infusion. Some patients exhibited vomiting, with or without nausea. With 1 exception (described below), a single dose of a non-steroidal agent such as paracetamol was sufficient to resolve these symptoms. Modest, transient cardiovascular effects were observed, consistent with, and sharing the time course of, the fever. No other adverse effects were reported.

At this late stage of cervical cancer, 1 year survival is typically 10-15% of patients and no tumor therapy has ever been effective. Indeed, Patient 2 was a young patient with very aggressive disease who passed away shortly after completing the trial.

Quantitative blood cultures were assessed on days 2, 3, and 5 post-administration. Of the 5 evaluable patients in this cohort, 4 exhibited no serum Listeria at any time and 1 had a very small amount (35 cfu) of circulating Listeria on day 2, with no detectable Listeria on day 3 or 5.

Patient 5 responded to initial vaccination with mild fever over the 48 hours subsequent to administration, and was treated with anti-inflammatory agents. On 1 occasion, the fever rose to moderate severity (at no time above 38.4° C.), after which she was given a course of ampicillin, which resolved the fever. During the antibiotic administration she experienced mild urticaria, which ended after antibiotic administration. Blood cultures were all sterile, cardiovascular data were within the range observed for other patients, and serum chemistry values were normal, showing that this patient had no Listerial disease. Further, the anergy panel indicated a robust response to 1/3 memory antigens, indicating the presence of functional immunity (similar to the other patients). Patient 5 subsequently evidenced a response similar to all other patients upon receiving the boost.

### Second Cohort and Overall Safety Observations

In both cohorts, minor and transient changes in liver function tests were observed following infusion. These changes were determined by the attending physician monitoring the trial to have no clinical significance, and were expected for a short-lived infection of bacteria that are rapidly removed from the systemic circulation to the liver and spleen. In general, all the safety indicators described in the Methods section above displayed little or no net change, indicative of an excellent safety profile. The side effect profile in this cohort was virtually identical to that seen in the in the initial cohort and appeared to be a dose independent series of symptoms related to the consequences of cytokines and similar agents that occur consequent to the induction of an iatrogenic infection. No serum Listeria was observed at any time and no dose limiting toxicity was observed in either cohort.

### Efficacy—First Cohort

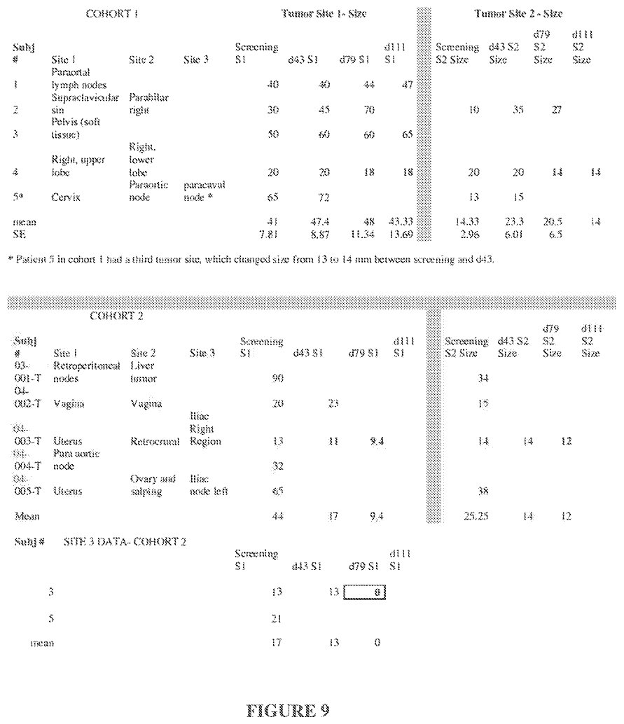

The following indications of efficacy were observed in the 3 patients in the first cohort that finished the trial: (FIG. 9).

Patient 1 entered the trial with 2 tumors of 20 mm each, which shrunk to 18 and 14 mm over the course of the trial, indicating therapeutic efficacy of the immunotherapy. In addition, patient 1 entered the trial with a Karnofsky Performance Index of 70, which rose to 90 after dosing. In the Safety Review Panel meeting, Sinis̆a Radulovic, the chairman of the Department of Oncology, Institute for Oncology and Radiology, Belgrade, Serbia presented the results to a representative of the entity conducting the trials; Michael Kurman, an independent oncologist who works as a consultant for the entity; Kevin Ault, an academic gynecologic oncologist at Emory University who conducted the phase III Gardasil trials for Merck and the Cervarix trials for Glaxo SmithKline; and Tate Thigpen, a founder of the Gynecologic Oncology Group at NCI and professor of gynecologic oncology at the University of Mississippi. In the opinion of Dr. Radulovic, patient 1 exhibited a clinical benefit from treatment with the immunotherapy.

Before passing away, Patient 2 exhibited a mixed response, with 1/2 tumors shrinking.

Patient 3 enrolled with paraneoplastic disease, (an epiphenomenon of cancer wherein the overall debilitated state of the patient has other sequelae that are secondary to the cancer), including an elevation of platelet count to 936×10⁹/ml. The count decreased to 405×10⁹/ml, approximately a normal level, following the first dose.

Patient 4 entered the trial with 2 tumors of 20 mm each, which shrunk to 18 and 14 mm over the course of the trial, indicating therapeutic efficacy of the immunotherapy. Patient 4 exhibited a weight gain of 1.6 Kg and an increased hemoglobin count of approximately 10% between the first and second doses.

### Efficacy—Second Cohort and General Observations

In the lowest dose cohort, 2 patients demonstrated the shrinkage of tumors. The timing of this effect was consistent with that observed in immunological responses, in that it followed chronologically development of the immune response. One of the 2 patients in the second cohort evaluated so far for tumor burden exhibited a dramatic tumor load reduction at a post-vaccination time point. At the start of the trial, this patient had 3 tumors of 13, 13, and 14 mm After the 2 doses of the immunotherapy, 2 of the tumor had shrunk to 9.4 and 12 mm, and the third was no longer detectable.

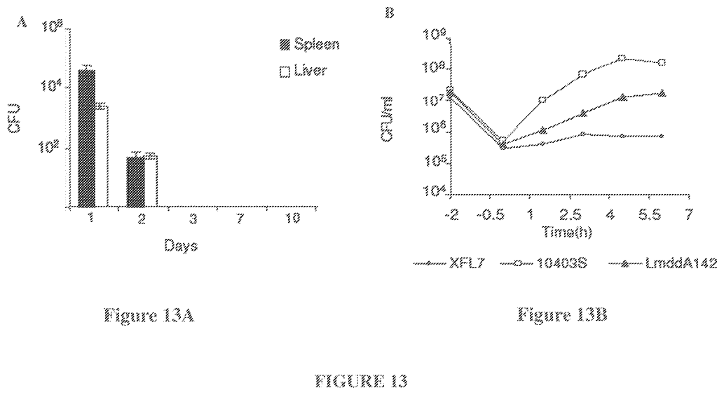

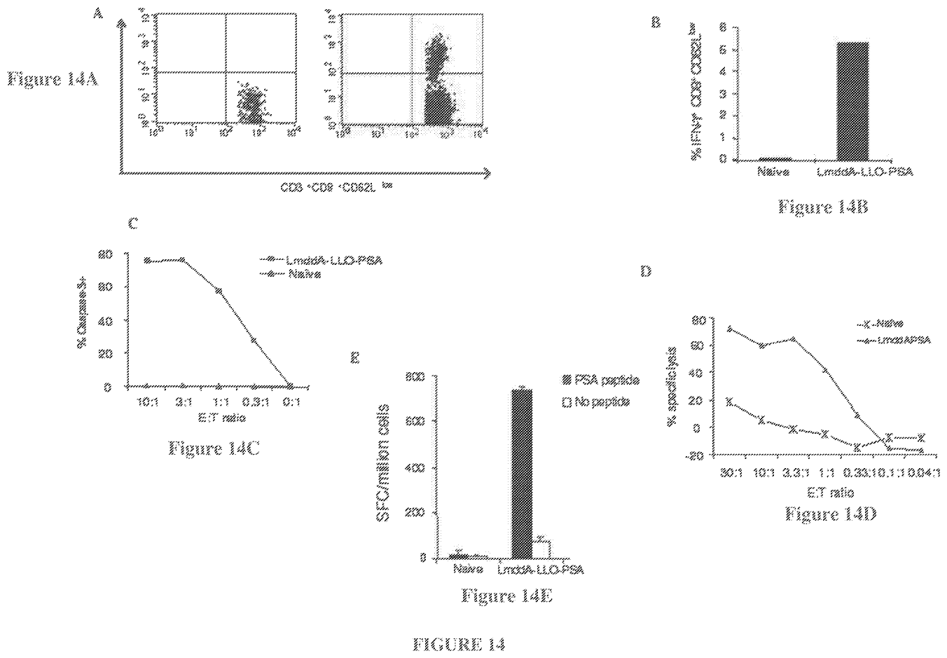

Tumors loads for the 2 cohorts are depicted in FIG. 13B. In summary, even relatively low doses of LM-LLO-E7, administered in a therapeutic regimen containing a priming injection and a single boost, achieved 3 objective responses out of 6 patients for whom data has been collected.

### Discussion

At this late stage of cervical cancer, 1 year survival is typically 10-15% of patients and no tumor therapy has ever been effective. No treatment has shown to be effective in reversing stage IVB cervical cancer. Despite the difficulty of treating cervical cancer at this stage, an anti-tumor effect was observed in 2/6 patients. In addition, other indications of efficacy were observed in patients that finished the trial, as described hereinabove.

Thus, LM-LLO-E7 is safe in human subjects and improves clinical indicators of cervical cancer patients, even when administered at relatively low doses. Additional positive results are likely to be observed when the dose and number of booster vaccinations is increased; and/or when antibiotics are administered in smaller doses or at a later time point after infusion. Pre-clinical studies have shown that a dose increase of a single order of magnitude can cause dramatic changes in response rate (e.g. a change from 0% response rate to 50-100% complete remission rate. Additional booster doses are also very likely to further enhance the immune responses obtained. Moreover, the positive effects of the therapeutic immune response observed are likely to continue with the passage of additional time, as the immune system continues to attack the cancer.

### Example 7: Construction of Attenuated Listeria Strain-LmddΔactA and Insertion of the Human Klk3 Gene in Frame to the Hly Gene in the Lmdd and Lmdda Strains

### Materials and Methods

A recombinant Lm was developed that secretes PSA fused to tLLO (Lm-LLO-PSA), which elicits a potent PSA-specific immune response associated with regression of tumors in a mouse model for prostate cancer, wherein the expression of tLLO-PSA is derived from a plasmid based on pGG55 (Table 2), which confers antibiotic resistance to the vector. We recently developed a new strain for the PSA immunotherapy based on the pADV142 plasmid, which has no antibiotic resistance markers, and referred as LmddA-142 (Table 3). This new strain is 10 times more attenuated than Lm-LLO-PSA. In addition, LmddA-142 was slightly more immunogenic and significantly more efficacious in regressing PSA expressing tumors than the Lm-LLO-PSA.

TABLE 2
Plasmids and strains

| Plasmids | Features |
|---|---|
| pGG55 | pAM401/pGB354 shuttle plasmid with gram(−) and |
| | gram(+) cm resistance, LLO-E7 expression |
| | cassette and a copy of Lm prfA gene |
| pTV3 | Derived from pGG55 by deleting cm genes and |
| | inserting the Lm dal gene |
| pADV119 | Derived from pTV3 by deleting the prfA gene |
| pADV134 | Derived from pADV119 by replacing the Lm dal |
| | gene by the Bacillus dal gene |
| pADV142 | Derived from pADV134 by replacing HPV16 e7 |
| | with klk3 |
| pADV168 | Derived from pADV134 by replacing HPV16 e7 |
| | with hmw-maa₂₁₆₀₋₂₂₅₈ |
| Strains | Genotype |
| 10403S | Wild-type Listeria monocytogenes:: str |
| XFL-7 | 10403S prfA⁽⁻⁾ |
| Lmdd | 10403S dal⁽⁻⁾ dat⁽⁻⁾ |
| LmnddA | 10403S dal⁽⁻⁾ dat⁽⁻⁾ actA⁽⁻⁾ |
| LmddA-134 | 10403S dal⁽⁻⁾ dat⁽⁻⁾ actA⁽⁻⁾ pADV134 |
| LmddA-142 | 10403S dal⁽⁻⁾ dat⁽⁻⁾ actA⁽⁻⁾ pADV142 |
| Lmdd-143 | 10403S dal⁽⁻⁾ dat⁽⁻⁾ with klk3 fused to the hly |
| | gene in the chromosome |
| LmddA-143 | 10403S dal⁽⁻⁾ dat⁽⁻⁾ actA⁽⁻⁾ with klk3 fused to |
| | the hly gene in the chromosome |
| LmddA-168 | 10403S dal⁽⁻⁾ dat⁽⁻⁾ actA⁽⁻⁾ pADV168 |
| Lmdd-143/134 | Lmdd-143 pADV134 |
| LmddA-143/134 | LmddA-143 pADV134 |
| Lmdd-143/168 | Lmdd-143 pADV168 |
| LmddA-143/168 | LmddA-143 pADV168 |

The sequence of the plasmid pAdv142 (6523 bp) was as follows:

cggagtgtatactggcttactatgttggcactgatgagggtgtcagtgaagtgcttcatgtggcaggagaaaaaaggctgcaccggtgc gtcagcagaatatgtgatacaggatatattccgcttcctcgctcactgactcgctacgctcggtcgttcgactgcggcgagcggaaatg gcttacgaacggggcggagatttcctggaagatgccaggaagatacttaacagggaagtgagagggccgcggcaaagccgtttttcc ataggctccgcccccctgacaagcatcacgaaatctgacgctcaaatcagtggtggcgaaacccgacaggactataaagataccagg cgtttccccctggcggctccctcgtgcgctctcctgttcctgcctttcggtttaccggtgtcattccgctgttatggccgcgtttgtctcattc cacgcctgacactcagttccgggtaggcagttcgctccaagctggactgtatgcacgaaccccccgttcagtccgaccgctgcgcctt atccggtaactatcgtcttgagtccaacccggaaagacatgcaaaagcaccactggcagcagccactggtaattgatttagaggagtta gtcttgaagtcatgcgccggttaaggctaaactgaaaggacaagttttggtgactgcgctcctccaagccagttacctcggttcaaagag ttggtagctcagagaaccttcgaaaaaccgccctgcaaggcggttttttcgttttcagagcaagagattacgcgcagaccaaaacgatct caagaagatcatcttattaatcagataaaatatttctagccctcctttgattagtatattcctatcttaaagttacttttatgtggaggcattaaca tttgttaatgacgtcaaaaggatagcaagactagaataaagctataaagcaagcatataatattgcgtttcatctttagaagcgaatttcgc caatattataattatcaaaagagaggggtggcaaacggtatttggcattattaggttaaaaaatgtagaaggagagtgaaacccatgaaa aaaataatgctagtttttattacacttatattagttagtctaccaattgcgcaacaaactgaagcaaaggatgcatctgcattcaataaagaa aattcaatttcatccatggcaccaccagcatctccgcctgcaagtcctaagacgccaatcgaaaagaaacacgcggatgaaatcgataa gtatatacaaggattggattacaataaaaacaatgtattagtataccacggagatgcagtgacaaatgtgccgccaagaaaaggttaca aagatggaaatgaatatattgttgtggagaaaaagaagaaatccatcaatcaaaataatgcagacattcaagttgtgaatgcaatttcgag cctaacctatccaggtgctctcgtaaaagcgaattcggaattagtagaaaatcaaccagatgttctccctgtaaaacgtgattcattaaca ctcagcattgatttgccaggtatgactaatcaagacaataaaatagttgtaaaaaatgccactaaatcaaacgttaacaacgcagtaaata cattagtggaaagatggaatgaaaaatatgctcaagcttatccaaatgtaagtgcaaaaattgattatgatgacgaaatggcttacagtga atcacaattaattgcgaaataggtacagcatttaaagctgtaaataatagcttgaatgtaaacttcggcgcaatcagtgaagggaaaatg caagaagaagtcattagattaaacaaatttactataacgtgaatgttaatgaacctacaagaccaccagattatcggcaaagctgttact aaagagcagttgcaagcgcttggagtgaatgcagaaaatcctcctgcatatatctcaagtgtggcgtatggccgtcaagtttatttgaaat tatcaactaattcccatagtactaaagtaaaagctgatttgatgctgccgtaagcggaaaatctgtctcaggtgatgtagaactaacaaat atcatcaaaaattcttccttcaaagccgtaatttacggaggttccgcaaaagatgaagttcaaatcatcgacggcaacctcggagacttac gcgatattagaaaaaaggcgctactataatcgagaaacaccaggagacccattgcttatacaacaaacacctaaaagacaatgaatta gctgttattaaaaacaactcagaatatattgaaacaacttcaaaagcttatacagatggaaaaattaacatcgatcactctggaggatacgt tgctcaattcaacatttcagggatgaagtaaattatgatctcgagattgtgggaggctgggagtgcgagaagcattcccaaccctggca ggtgcttgtggcctctcgtggcagggcagtctgcggcggtgactggtgcacccccagtgggtcctcacagctgcccactgcatcagg aacaaaagcgtgatcttgctgggtcggcacagcctgatcatcctgaagacacaggccaggtatttcaggtcagccacagatcccaca cccgctctacgatatgagcctcctgaagaatcgattcctcaggccaggtgatgactccagccacgacctcatgctgctccgcctgtcag agcctgccgagctcacggatgctgtgaaggtcatggacctgcccacccaggagccagcactggggaccacctgctacgcctcaggc tggggcagcattgaaccagaggagttcttgaccccaaagaaacttcagtgtgtggacctccatgttatttccaatgacgtgtgtgcgcaa gttcaccctcagaaggtgaccaagttcatgctgtgtgctggacgctggacagggggcaaaagcacctgctcgggtgattctgggggc ccacttgtctgttatggtgtgcttcaaggtatcacgtcatggggcagtgaaccatgtgccctgcccgaaaggccaccctgtacaccaag gtggtgcattaccggaagtggatcaaggacaccatcgtggccaaccccTAAcccgggccactaactcaacgctagtagtggattta atcccaaatgagccaacagaaccagaaccagaaacagaacaagtaacattggagttagaaatggaagaagaaaaaagcaatgatttc gtgtgaataatgcacgaaatcattgcttattatttaaaaagcgatatactagatataacgaaacaacgaactgaataaagaatacaaaaaa agagccacgaccagttaaagcctgagaaactttaactgcgagccttaattgattaccaccaatcaattaaagaagtcgagacccaaaatt tggtaaagtatttaattactttattaatcagatacttaaatatctgtaaacccattatatcgggtttttgaggggatttcaagtctttaagaagata ccaggcaatcaattaagaaaaacttagttgattgccttttttgttgtgattcaactttgatcgtagcttctaactaattaattttcgtaagaaagg agaacagctgaatgaatatcccattgagtagaaactgtgcttcatgacggcttgttaaagtacaaatttaaaaatagtaaaattcgctcaat cactaccaagccaggtaaaagtaaaggggctatattgcgtatcgctcaaaaaaaagcatgattggcggacgtggcgttgactgacttc cgaagaagcgattcacgaaaatcaagatacatttacgcattggacaccaaacgatatcgttatggtacgtatgcagacgaaaaccgttc atacactaaaggacattctgaaaacaatttaagacaaatcaataccttctttattgattttgatattcacacggaaaaagaaactatttcagca agcgatattttaacaacagctattgatttaggttttatgcctacgttaattatcaaatctgataaaggttatcaagcatattttgttttagaaacg ccagtctatgtgacttcaaaatcagaatttaaatctgtcaaagcagccaaaataatctcgcaaaatatccgagaatattaggaaagtattg ccagttgatctaacgtgcaatcattagggattgctcgtataccaagaacggacaatgtagaattattgatcccaattaccgttattattcaa agaatggcaagattggtctttcaaacaaacagataataagggctttactcgttcaagtctaacggttttaagcggtacagaaggcaaaaa acaagtagatgaaccctggataatctcttattgcacgaaacgaaattacaggagaaaagggatagtagggcgcaatagcgttatgata ccctctctttagcctactttagttcaggctattcaatcgaaacgtgcgaatataatatgtttgagtttaataatcgattagatcaacccttagaa gaaaaagaagtaatcaaaattgttagaagtgcctattcagaaaactatcaaggggctaatagggaatacattaccattctttgcaaagctt gggtatcaagtgatttaaccagtaaagatttatagtccgtcaagggtggataaattcaagaaaaaaagaagcgaacgtcaacgtgaca tttgtcagaatggaaagaagatttaatggcttatattagcgaaaaaagcgatgtatacaagccttatttagcgacgaccaaaaaagagatt agagaagtgctaggcattcctgaacggacattagataaattgctgaaggtactgaaggcgaatcaggaaattttctttaagattaaacca ggaagaaatggtggcattcaacttgctagtgttaaatcattgttgctatcgatcattaaattaaaaaaagaagaacgagaaagctatataa aggcgctgacagcttcgtttaatttagaacgtacatttattcaagaaactctaaacaaattggcagaacgccccaaaacggacccacaac tcgatttgtttagctacgatacaggctgaaaataaaacccgcactatgccattacatttatatctatgatacgtgtttgatttattgctggcta gcttaattgcttatatttacctgcaataaaggatttcttacttccattatactcccattttccaaaaacatacggggaacacgggaacttattgt acaggccacctcatagttaatggtttcgagccttcctgcaatctcatccatggaaatatattcatccccctgccggcctattaatgtgactttt gtgcccggcggatattcctgatccagctccaccataaattggtccatgcaaattcggccggcaattttcaggcgttttcccttcacaagga tgtcggtccctttcaattttcggagccagccgtccgcatagcctacaggcaccgtcccgatccatgtgtctttttccgctgtgtactcggct ccgtagctgacgctctcgccttttctgatcagtttgacatgtgacagtgtcgaatgcagggtaaatgccggacgcagctgaaacggtatc tcgtccgacatgtcagcagacgggcgaaggccatacatgccgatgccgaatctgactgcattaaaaaagccttttttcagccggagtcc agcggcgctgttcgcgcagtggaccattagattctttaacggcagcggagcaatcagctctttaaagcgctcaaactgcattaagaaat agcctctttctttttcatccgctgtcgcaaaatgggtaaatacccctttgcactttaaacgagggttgcggtcaagaattgccatcacgttct gaacttcttcctctgtttttacaccaagtctgttcatccccgtatcgaccttcagatgaaaatgaagagaaccttttttcgtgtggcgggctgc ctcctgaagccattcaacagaataacctgttaaggtcacgtcatactcagcagcgattgccacatactccgggggaaccgcgccaagc accaatataggcgccttcaatccctttttgcgcagtgaaatcgcttcatccaaaatggccacggccaagcatgaagcacctgcgtcaag agcagcctttgctgtttctgcatcaccatgcccgtaggcgtttgctttcacaactgccatcaagtggacatgttcaccgatatgttttttcata ttgctgacattttcctttatcgcggacaagtcaatttccgcccacgtatctctgtaaaaaggttttgtgctcatggaaaactcctctcttttttca gaaaatcccagtacgtaattaagtatttgagaattaattttatattgattaatactaagtttacccagttttcacctaaaaaacaaatgatgaga taatagctccaaaggctaaagaggactataccaactatttgttaattaa (SEQ ID NO: 41). This plasmid was sequenced at Genewiz facility from the E. coli strain on 2-20-08.

The strain Lm dal dat (Lmdd) was attenuated by the irreversible deletion of the virulence factor, ActA. An in-frame deletion of actA in the Lmdaldat (Lmdd) background was constructed to avoid any polar effects on the expression of downstream genes. The Lm dal dat ΔactA contains the first 19 amino acids at the N-terminal and 28 amino acid residues of the C-terminal with a deletion of 591 amino acids of ActA.

The actA deletion mutant was produced by amplifying the chromosomal region corresponding to the upstream (657 bp-oligo's Adv 271/272) and downstream (625 bp-oligo's Adv 273/274) portions of actA and joining by PCR. The sequence of the primers used for this amplification is given in the Table 3. The upstream and downstream DNA regions of actA were cloned in the pNEB193 at the EcoRI/PstI restriction site and from this plasmid, the EcoRI/PstI was further cloned in the temperature sensitive plasmid pKSV7, resulting in ΔactA/pKSV7 (pAdv120).

TABLE 3
Sequence of primers that was used for the amplification of DNA
sequences upstream and downstream of actA

| Primer | Sequence | SEQ ID NO: |
|---|---|---|
| Adv271-actAF1 | cg GAATTCGGATCCgcgccaaatcattggttgattg | 42 |
| Adv272-actAR1 | gcgaGTCGACgtcggggttaatcgtaatgcaattggc | 43 |
| Adv273-actAF2 | gcgaGTCGACccatacgacgttaattcttgcaatg | 44 |
| Adv274-actAR2 | gataCTGCAGGGATCCttcccttctcggtaatcagtcac | 45 |

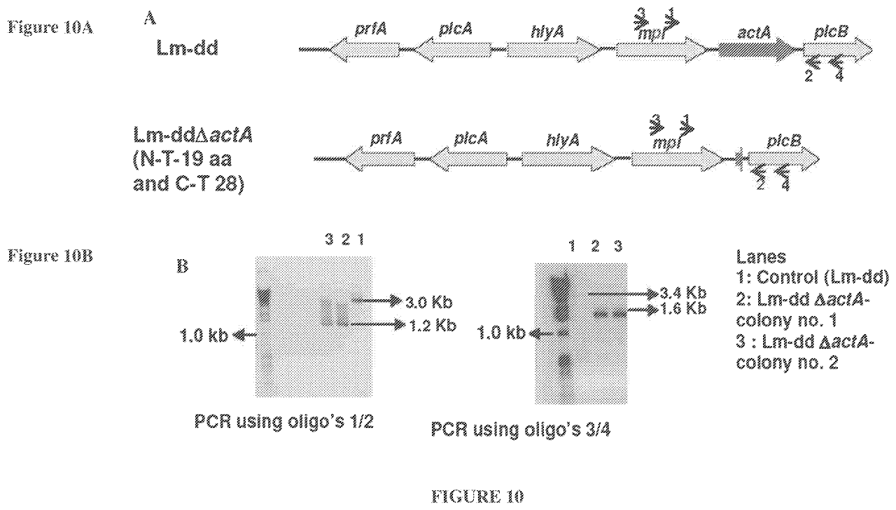

The deletion of the gene from its chromosomal location was verified using primers that bind externally to the actA deletion region, which are shown in FIG. 10A and FIG. 10B as primer 3 (Adv 305-tgggatggccaagaaattc, SEQ ID NO: 46) and primer 4 (Adv304-ctaccatgtcttccgttgcttg; SEQ ID NO: 47). The PCR analysis was performed on the chromosomal DNA isolated from Lmdd and LmddΔactA. The sizes of the DNA fragments after amplification with two different sets of primer pairs 1/2 and 3/4 in Lmdd chromosomal DNA was expected to be 3.0 Kb and 3.4 Kb. On the other hand, the expected sizes of PCR using the primer pairs 1/2 and 3/4 for the LmdddΔactA was 1.2 Kb and 1.6 Kb. Thus, PCR analysis in FIG. 10A and FIG. 10B confirms that the 1.8 kb region of actA was deleted in the LmdddΔactA strain. DNA sequencing was also performed on PCR products to confirm the deletion of actA containing region in the strain, LmdddΔactA.

### Example 8: Construction of the Antibiotic-Independent Episomal Expression System for Antigen Delivery by Lm Vectors

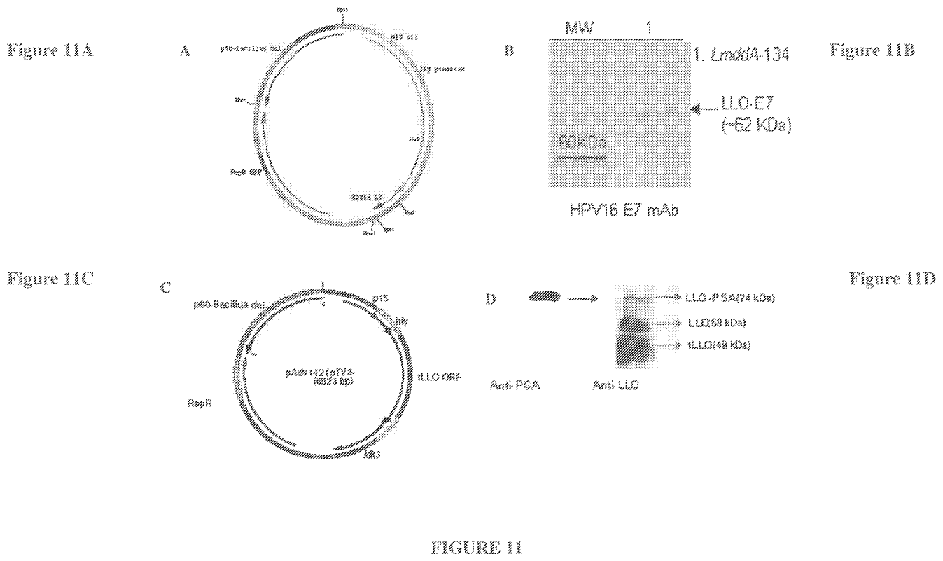

The antibiotic-independent episomal expression system for antigen delivery by Lm vectors (pAdv142) is the next generation of the antibiotic-free plasmid pTV3 (Verch et al., Infect Immun, 2004. 72(11):6418-25, incorporated herein by reference). The gene for virulence gene transcription activator, prfA was deleted from pTV3 since Listeria strain Lmdd contains a copy of prfA gene in the chromosome. Additionally, the cassette for p60-Listeria dal at the NheI/PacI restriction site was replaced by p60-Bacillus subtilis dal resulting in plasmid pAdv134 (FIG. 11A). The similarity of the Listeria and Bacillus dal genes is ˜30%, virtually eliminating the chance of recombination between the plasmid and the remaining fragment of the dal gene in the Lmdd chromosome. The plasmid pAdv134 contained the antigen expression cassette tLLO-E7. The LmddA strain was transformed with the pADV134 plasmid and expression of the LLO-E7 protein from selected clones confirmed by Western blot (FIG. 11B). The Lmdd system derived from the 10403S wild-type strain lacks antibiotic resistance markers, except for the Lmdd streptomycin resistance.

Further, pAdv134 was restricted with XhoI/XmaI to clone human PSA, klk3 resulting in the plasmid, pAdv142. The new plasmid, pAdv142 (FIG. 11C, Table 2) contains Bacillus dal (B-Dal) under the control of Listeria p60 promoter. The shuttle plasmid, pAdv142 complemented the growth of both E. coli ala drx MB2159 as well as Listeria monocytogenes strain Lmdd in the absence of exogenous D-alanine. The antigen expression cassette in the plasmid pAdv142 consists of hly promoter and LLO-PSA fusion protein (FIG. 11C).

The plasmid pAdv142 was transformed to the Listeria background strains, LmddactA strain resulting in Lm-ddA-LLO-PSA. The expression and secretion of LLO-PSA fusion protein by the strain, Lm-ddA-LLO-PSA was confirmed by Western Blot using anti-LLO and anti-PSA antibody (FIG. 11D). There was stable expression and secretion of LLO-PSA fusion protein by the strain, Lm-ddA-LLO-PSA after two in vivo passages.

### Example 9: In Vitro and In Vivo Stability of the Strain LmddA-LLO-PSA

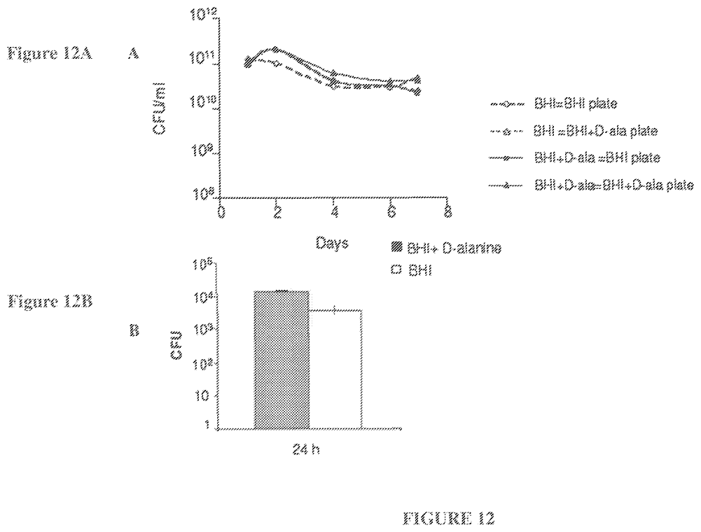

The in vitro stability of the plasmid was examined by culturing the LmddA-LLO-PSA Listeria strain in the presence or absence of selective pressure for eight days. The selective pressure for the strain LmddA-LLO-PSA is D-alanine. Therefore, the strain LmddA-LLO-PSA was passaged in Brain-Heart Infusion (BHI) and BHI+100 μg/ml D-alanine. CFUs were determined for each day after plating on selective (BHI) and non-selective (BHI+D-alanine) medium. It was expected that a loss of plasmid will result in higher CFU after plating on non-selective medium (BHI+D-alanine). As depicted in FIG. 12A, there was no difference between the number of CFU in selective and non-selective medium. This suggests that the plasmid pAdv142 was stable for at least 50 generations, when the experiment was terminated.

Plasmid maintenance in vivo was determined by intravenous injection of 5×10⁷ CFU LmddA-LLO-PSA, in C57BL/6 mice. Viable bacteria were isolated from spleens homogenized in PBS at 24 h and 48 h. CFUs for each sample were determined at each time point on BHI plates and BHI+100 mg/ml D-alanine. After plating the splenocytes on selective and non-selective medium, the colonies were recovered after 24 h. Since this strain is highly attenuated, the bacterial load is cleared in vivo in 24 h. No significant differences of CFUs were detected on selective and non-selective plates, indicating the stable presence of the recombinant plasmid in all isolated bacteria (FIG. 12B).

### Example 10: In Vivo Passaging, Virulence and Clearance of the Strain LmddA-142 (LmddA-LLO-PSA)

LmddA-142 is a recombinant Listeria strain that secretes the episomally expressed tLLO-PSA fusion protein. To determine a safe dose, mice were immunized with LmddA-LLO-PSA at various doses and toxic effects were determined. LmddA-LLO-PSA caused minimum toxic effects (data not shown). The results suggested that a dose of 10⁸ CFU of LmddA-LLO-PSA was well tolerated by mice. Virulence studies indicate that the strain LmddA-LLO-PSA was highly attenuated.

The in vivo clearance of LmddA-LLO-PSA after administration of the safe dose, 10⁸ CFU intraperitoneally in C57BL/6 mice, was determined. There were no detectable colonies in the liver and spleen of mice immunized with LmddA-LLO-PSA after day 2. Since this strain is highly attenuated, it was completely cleared in vivo at 48 h (FIG. 13A).

To determine if the attenuation of LmddA-LLO-PSA attenuated the ability of the strain LmddA-LLO-PSA to infect macrophages and grow intracellularly, a cell infection assay was performed. Mouse macrophage-like cell line such as J774A.1, were infected in vitro with Listeria constructs and intracellular growth was quantified. The positive control strain, wild type Listeria strain 10403S grows intracellularly, and the negative control XFL7, a prfA mutant, cannot escape the phagolysosome and thus does not grow in J774 cells. The intracytoplasmic growth of LmddA-LLO-PSA was slower than 10403S due to the loss of the ability of this strain to spread from cell to cell (FIG. 13B). The results indicate that LmddA-LLO-PSA has the ability to infect macrophages and grow intracytoplasmically.

### Example 11: Immunogenicity of the Strain-LmddA-LLO-PSA in C57BL/6 Mice

The PSA-specific immune responses elicited by the construct LmddA-LLO-PSA in C57BL/6 mice were determined using PSA tetramer staining. Mice were immunized twice with LmddA-LLO-PSA at one week intervals and the splenocytes were stained for PSA tetramer on day 6 after the boost. Staining of splenocytes with the PSA-specific tetramer showed that LmddA-LLO-PSA elicited 23% of PSA tetramer⁺CD8⁺CD62L^{low} cells (FIG. 14A). The functional ability of the PSA-specific T cells to secrete IFN-γ after stimulation with PSA peptide for 5 h was examined using intracellular cytokine staining. There was a 200-fold increase in the percentage of CD8⁺CD62L^{low}IFN-γ secreting cells stimulated with PSA peptide in the LmddA-LLO-PSA group compared to the naïve mice (FIG. 14B), indicating that the LmddA-LLO-PSA strain is very immunogenic and primes high levels of functionally active PSA CD8+ T cell responses against PSA in the spleen.

To determine the functional activity of cytotoxic T cells generated against PSA after immunizing mice with LmddA-LLO-PSA, we tested the ability of PSA-specific CTLs to lyse cells EL4 cells pulsed with H-2D^{b} peptide in an in vitro assay. A FACS-based caspase assay (FIG. 14C) and Europium release (FIG. 14D) were used to measure cell lysis. Splenocytes of mice immunized with LmddA-LLO-PSA contained CTLs with high cytolytic activity for the cells that display PSA peptide as a target antigen.

Elispot was performed to determine the functional ability of effector T cells to secrete IFN-γ after 24 h stimulation with antigen. Using ELISpot, a 20-fold increase in the number of spots for IFN-γ in splenocytes from mice immunized with LmddA-LLO-PSA stimulated with specific peptide when compared to the splenocytes of the naïve mice was observed (FIG. 14E).

### Example 12: Immunization with the LmddA-142 Strains Induces Regression of a Tumor Expressing PSA and Infiltration of the Tumor by PSA-Specific CTLs

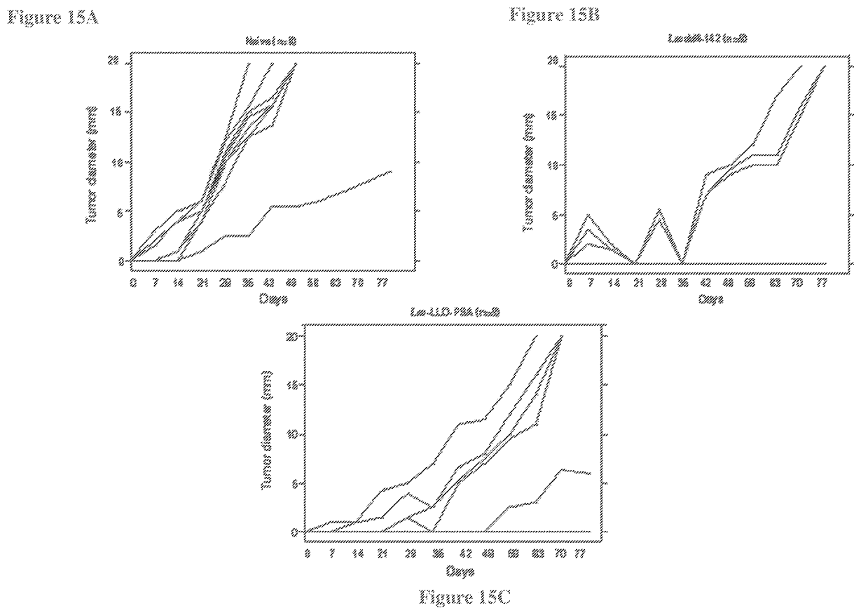

The therapeutic efficacy of the construct LmddA-142 (LmddA-LLO-PSA) was determined using a prostrate adenocarcinoma cell line engineered to express PSA (Tramp-C1-PSA (TPSA); Shahabi et al., 2008). Mice were subcutaneously implanted with 2×10⁶ TPSA cells. When tumors reached the palpable size of 4-6 mm, on day 6 after tumor inoculation, mice were immunized three times at one week intervals with 10⁸ CFU LmddA-142, 10⁷ CFU Lm-LLO-PSA (positive control) or left untreated. The naïve mice developed tumors gradually (FIG. 15A). The mice immunized with LmddA-142 were all tumor-free until day 35 and gradually 3 out of 8 mice developed tumors, which grew at a much slower rate as compared to the naïve mice (FIG. 15B). Five out of eight mice remained tumor free through day 70. As expected, Lm-LLO-PSA-vaccinated mice had fewer tumors than naïve controls and tumors developed more slowly than in controls (FIG. 15C). Thus, the construct LmddA-LLO-PSA could regress 60% of the tumors established by TPSA cell line and slow the growth of tumors in other mice. Cured mice that remained tumor free were rechallenged with TPSA tumors on day 68.

Immunization of mice with the LmddA-142 can control the growth and induce regression of 7-day established Tramp-C1 tumors that were engineered to express PSA in more than 60% of the experimental animals (FIG. 15B), compared to none in the untreated group (FIG. 15A). The LmddA-142 was constructed using a highly attenuated vector (LmddA) and the plasmid pADV142 (Table 2).

Further, the ability of PSA-specific CD8 lymphocytes generated by the LmddA-LLO-PSA construct to infiltrate tumors was investigated. Mice were subcutaneously implanted with a mixture of tumors and matrigel followed by two immunizations at seven day intervals with naïve or control (Lm-LLO-E7) Listeria, or with LmddA-LLO-PSA. Tumors were excised on day 21 and were analyzed for the population of CD8⁺CD62L^{low} PSA^{tetramer+} and CD4⁺ CD25⁺FoxP3⁺ regulatory T cells infiltrating in the tumors.

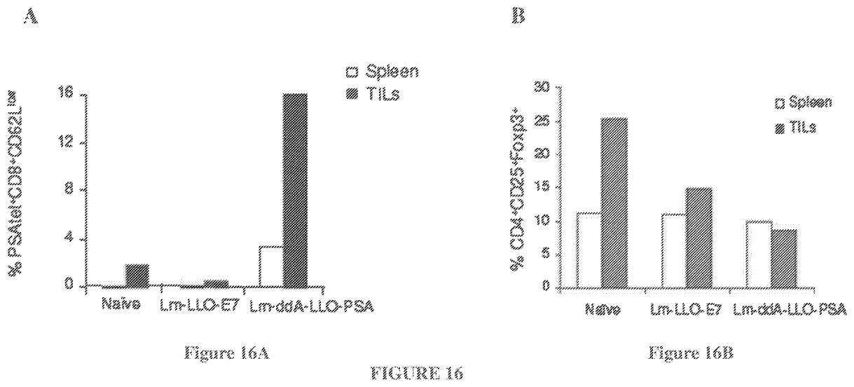

A very low number of CD8⁺CD62L^{low} PSA^{tetramer+} tumor infiltrating lymphocytes (TILs) specific for PSA that were present in the both naïve and Lm-LLO-E7 control immunized mice was observed. However, there was a 10-30-fold increase in the percentage of PSA-specific CD8⁺CD62L^{low} PSA^{tetramer+} TILs in the mice immunized with LmddA-LLO-PSA (FIG. 7A). Interestingly, the population of CD8⁺CD62L^{low} PSA^{tetramer+} cells in spleen was 7.5 fold less than in tumor (FIG. 16A).

In addition, the presence of CD4⁺/CD25⁺/Foxp3⁺ T regulatory cells (Tregs) in the tumors of untreated mice and Listeria immunized mice was determined. Interestingly, immunization with Listeria resulted in a considerable decrease in the number of CD4⁺ CD25⁺FoxP3⁺ T-regs in tumor but not in spleen (FIG. 16B). However, the construct LmddA-LLO-PSA had a stronger impact in decreasing the frequency of CD4⁺ CD25⁺FoxP3⁺ T-regs in tumors when compared to the naïve and Lm-LLO-E7 immunized group (FIG. 16B).

Thus, the LmddA-142 immunotherapy can induce PSA-specific CD8+ T cells that are able to infiltrate the tumor site (FIG. 16A). Interestingly, immunization with LmddA-142 was associated with a decreased number of regulatory T cells in the tumor (FIG. 16B), probably creating a more favorable environment for an efficient anti-tumor CTL activity.

### Example 13: Lmdd-143 and LmddA-143 Secretes a Functional LLO Despite the PSA Fusion

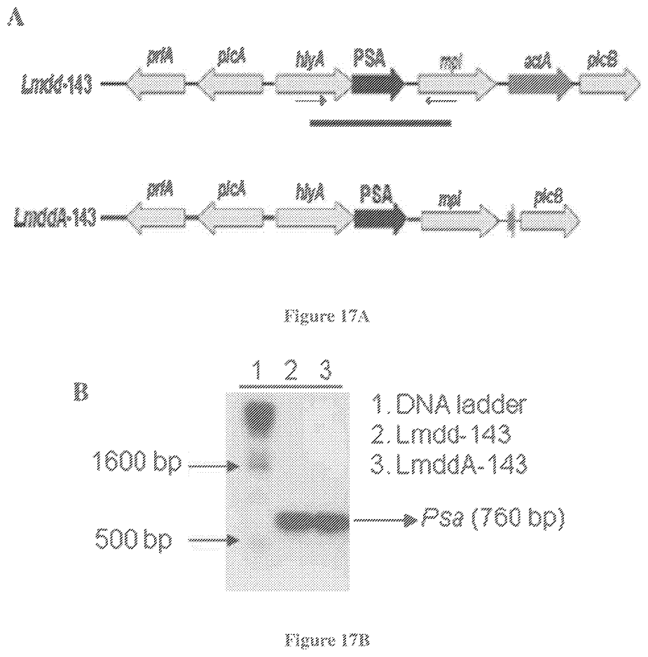

The Lmdd-143 and LmddA-143 contain the full-length human klk3 gene, which encodes the PSA protein, inserted by homologous recombination downstream and in frame with the hly gene in the chromosome. These constructs were made by homologous recombination using the pKSV7 plasmid (Smith and Youngman, Biochimie 1992; 74 (7-8) p705-711), which has a temperature-sensitive replicon, carrying the hly-klk3-mpl recombination cassette. Because of the plasmid excision after the second recombination event, the antibiotic resistance marker used for integration selection is lost. Additionally, the actA gene is deleted in the LmddA-143 strain (FIG. 17A). The insertion of klk3 in frame with hly into the chromosome was verified by PCR (FIG. 17B) and sequencing (data not shown) in both constructs.

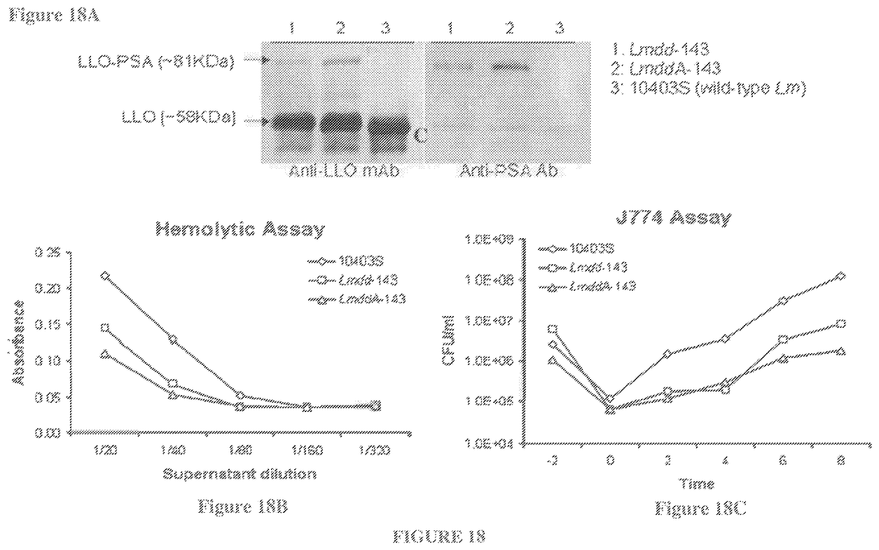

One important aspect of these chromosomal constructs is that the production of LLO-PSA would not completely abolish the function of LLO, which is required for escape of Listeria from the phagosome, cytosol invasion and efficient immunity generated by L. monocytogenes. Western-blot analysis of secreted proteins from Lmdd-143 and LmddA-143 culture supernatants revealed an ˜81 kDa band corresponding to the LLO-PSA fusion protein and an ˜60 kDa band, which is the expected size of LLO (FIG. 18A), indicating that LLO is either cleaved from the LLO-PSA fusion or still produced as a single protein by L. monocytogenes, despite the fusion gene in the chromosome. The LLO secreted by Lmdd-143 and LmddA-143 retained 50% of the hemolytic activity, as compared to the wild-type L. monocytogenes 10403S (FIG. 18B). In agreement with these results, both Lmdd-143 and LmddA-143 were able to replicate intracellularly in the macrophage-like J774 cell line (FIG. 18C).

### Example 14: Both Lmdd-143 and LmddA-143 Elicit Cell-Mediated Immune Responses Against the PSA Antigen

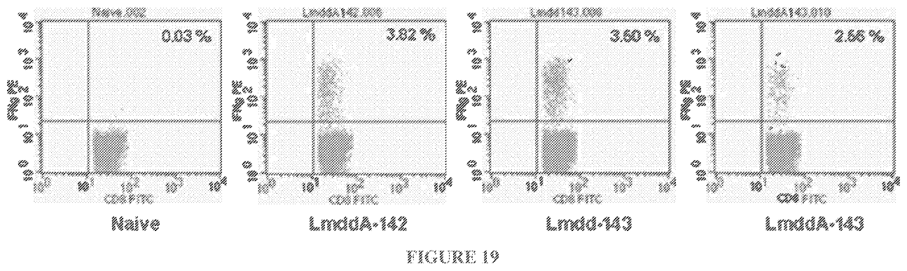

After showing that both Lmdd-143 and LmddA-143 were able to secrete PSA fused to LLO, the question of if these strains could elicit PSA-specific immune responses in vivo was investigated. C57Bl/6 mice were either left untreated or immunized twice with the Lmdd-143, LmddA-143 or LmddA-142. PSA-specific CD8+ T cell responses were measured by stimulating splenocytes with the PSA₆₅₋₇₄ peptide and intracellular staining for IFN-γ. As shown in FIG. 19, the immune response induced by the chromosomal and the plasmid-based vectors is similar

### Materials and Methods (Examples 15-20)

Oligonucleotides were synthesized by Invitrogen (Carlsbad, Calif.) and DNA sequencing was done by Genewiz Inc., South Plainfield, N.J. Flow cytometry reagents were purchased from Becton Dickinson Biosciences (BD, San Diego, Calif.). Cell culture media, supplements and all other reagents, unless indicated, were from Sigma (St. Louise, Mo.). Her2/neu HLA-A2 peptides were synthesized by EZbiolabs (Westfield, Ind.). Complete RPMI 1640 (C-RPMI) medium contained 2 mM glutamine, 0.1 mM non-essential amino acids, and 1 mM sodium pyruvate, 10% fetal bovine serum, penicillin/streptomycin, Hepes (25 mM). The polyclonal anti-LLO antibody was described previously and anti-Her2/neu antibody was purchased from Sigma.

### Mice and Cell Lines

All animal experiments were performed according to approved protocols by IACUC at the University of Pennsylvania or Rutgers University. FVB/N mice were purchased from Jackson laboratories (Bar Harbor, Me.). The FVB/N Her2/neu transgenic mice, which overexpress the rat Her2/neu onco-protein were housed and bred at the animal core facility at the University of Pennsylvania. The NT-2 tumor cell line expresses high levels of rat Her2/neu protein, was derived from a spontaneous mammary tumor in these mice and grown as described previously. DHFR-G8 (3T3/neu) cells were obtained from ATCC and were grown according to the ATCC recommendations. The EMT6-Luc cell line was a generous gift from Dr. John Ohlfest (University of Minnesota, Minn.) and was grown in complete C-RPMI medium. Bioluminescent work was conducted under guidance by the Small Animal Imaging Facility (SAIF) at the University of Pennsylvania (Philadelphia, Pa.).

### Listeria Constructs and Antigen Expression

Her2/neu-pGEM7Z was kindly provided by Dr. Mark Greene at the University of Pennsylvania and contained the full-length human Her2/neu (hHer2) gene cloned into the pGEM7Z plasmid (Promega, Madison Wis.). This plasmid was used as a template to amplify three segments of hHer-2/neu, namely, EC1, EC2, and IC1, by PCR using pfx DNA polymerase (Invitrogen) and the oligos indicated in Table 4.

TABLE 4
Primers for cloning of Human her-2-Chimera

| | | | Amino acid |
|---|---|---|---|
| | | Base pair | region or |
| | DNA sequence | region | junctions |
| Her-2- | TGATCTCGAGACCCACCTGGACATGCTC | 120-510 | 40-170 |
| Chimera | (SEQ ID NO: 48) | | |
| (F) | | | |
| HerEC1- | CTACCAGGACACGATTTTGTGGAAG- | 510/1077 | 170/359 |
| EC2F | AATATCCAGGAGTTTGCTGGCTGC | | |
| (Junction) | (SEQ ID NO: 49) | | |
| HerEC1- | GCAGCCAGCAAACTCCTGGATATT- | | |
| EC2R | CTTCCACAAAATCGTGTCCTGGTAG | | |
| (Junction) | (SEQ ID NO: 50) | | |
| HerEC2- | CTGCCACCAGCTGTGCGCCCGAGGG- | 1554/2034 | 518/679 |
| ICIF | CAGCAGAAGATCCGGAAGTACACGA | | |
| (Junction) | (SEQ ID NO: 51) | | |
| HerEC2- | TCGTGTACTTCCGGATCTTCTGCTGCCC | | |
| ICIR | TCGGGC GCACAGCTGGTGGCAG | | |
| (Junction) | (SEQ ID NO: 84) | | |
| Her-2- | GTGGCCCGGGTCTAGATTAGTCTAAGA | 2034-2424 | 679-808 |
| Chimera | GGCAGCCATAGG | | |
| (R) | (SEQ ID NO: 52) | | |

The Her-2/neu chimera construct was generated by direct fusion by the SOEing PCR method and each separate hHer-2/neu segment as templates. Primers are shown in Table 5.

TABLE 5

| | | Base | Amino |
|---|---|---|---|
| | | pair | acid |
| | DNA sequence | region | region |
| Her-2- | CCGCCTCGAGGCCGCGAGCACCCAAGTG | 58-979 | 20-326 |
| EC1(F) | (SEQ ID NO: 53) | | |
| Her-2- | CGCGACTAGTTTAATCCTCTGCTGTCACCTC | | |
| EC1(R) | (SEQ ID NO: 54) | | |
| Her-2- | CCGCCTCGAGTACCTTTCTACGGACGTG | 907-1504 | 303-501 |
| EC2(F) | (SEQ ID NO: 55) | | |
| Her-2- | CGCGACTAGTTTACTCTGGCCGGTTGGCAG | | |
| EC2(R) | (SEQ ID NO: 56) | | |
| Her-2- | CCGCCTCGAGCAGCAGAAGATCCGGAAGTAC | 2034- | 679-1081 |
| Her-2- | (SEQ ID NO: 57) | 3243 | |
| IC1(F) | | | |
| Her-2- | CGCGACTAGTTTAAGCCCCTTCGGAGGGTG | | |
| IC1(R) | (SEQ ID NO: 58) | | |

Sequence of Primers for Amplification of Different Segments Human Her2 Regions

ChHer2 gene was excised from pAdv138 using XhoI and SpeI restriction enzymes, and cloned in frame with a truncated, non-hemolytic fragment of LLO in the Lmdd shuttle vector, pAdv134. The sequences of the insert, LLO and hly promoter were confirmed by DNA sequencing analysis. This plasmid was electroporated into electro-competent actA, dal, dat mutant Listeria monocytogenes strain, LmddA and positive clones were selected on Brain Heart infusion (BHI) agar plates containing streptomycin (250 μg/ml). In some experiments similar Listeria strains expressing hHer2/neu (Lm-hHer2) fragments were used for comparative purposes. In all studies, an irrelevant Listeria construct (Lm-control) was included to account for the antigen independent effects of Listeria on the immune system. Lm-controls were based on the same Listeria platform as ADXS31-164 (LmddA-ChHer2), but expressed a different antigen such as HPV16-E7 or NY-ESO-1. Expression and secretion of fusion proteins from Listeria were tested. Each construct was passaged twice in vivo.

### Cytotoxicity Assay

Groups of 3-5 FVB/N mice were immunized three times with one week intervals with 1×10⁸ colony forming units (CFU) of Lm-LLO-ChHer2, ADXS31-164, Lm-hHer2 ICI or Lm-control (expressing an irrelevant antigen) or were left naive. NT-2 cells were grown in vitro, detached by trypsin and treated with mitomycin C (250 μg/ml in serum free C-RPMI medium) at 37° C. for 45 minutes. After 5 washes, they were co-incubated with splenocytes harvested from immunized or naïve animals at a ratio of 1:5 (Stimulator: Responder) for 5 days at 37° C. and 5% CO₂. A standard cytotoxicity assay was performed using europium labeled 3T3/neu (DHFR-G8) cells as targets according to the method previously described. Released europium from killed target cells was measured after 4 hour incubation using a spectrophotometer (Perkin Elmer, Victor²) at 590 nm. Percent specific lysis was defined as (lysis in experimental group-spontaneous lysis)/(Maximum lysis-spontaneous lysis).

Interferon-γSecretion by Splenocytes from Immunized Mice

Groups of 3-5 FVB/N or HLA-A2 transgenic mice were immunized three times with one week intervals with 1×10⁸ CFU of ADXS31-164, a negative Listeria control (expressing an irrelevant antigen) or were left naïve. Splenocytes from FVB/N mice were isolated one week after the last immunization and co-cultured in 24 well plates at 5×10⁶ cells/well in the presence of mitomycin C treated NT-2 cells in C-RPMI medium. Splenocytes from the HLA-A2 transgenic mice were incubated in the presence of 1 μM of HLA-A2 specific peptides or 1 μg/ml of a recombinant His-tagged ChHer2 protein, produced in E. coli and purified by a nickel based affinity chromatography system. Samples from supernatants were obtained 24 or 72 hours later and tested for the presence of interferon-γ (IFN-γ) using mouse IFN-γ Enzyme-linked immunosorbent assay (ELISA) kit according to manufacturer's recommendations.

### Tumor Studies in Her2 Transgenic Animals

Six weeks old FVB/N rat Her2/neu transgenic mice (9-14/group) were immunized 6 times with 5×10⁸ CFU of Lm-LLO-ChHer2, ADXS31-164 or Lm-control. They were observed twice a week for the emergence of spontaneous mammary tumors, which were measured using an electronic caliper, for up to 52 weeks. Escaped tumors were excised when they reached a size 1 cm² in average diameter and preserved in RNAlater at −20° C. In order to determine the effect of mutations in the Her2/neu protein on the escape of these tumors, genomic DNA was extracted using a genomic DNA isolation kit, and sequenced.

### Effect of ADXS31-164 on Regulatory T Cells in Spleens and Tumors

Mice were implanted subcutaneously (s.c.) with 1×10⁶ NT-2 cells. On days 7, 14 and 21, they were immunized with 1×10⁸ CFUs of ADXS31-164, LmddA-control or left naïve. Tumors and spleens were extracted on day 28 and tested for the presence of CD3⁺/CD4⁺/FoxP3⁺ Tregs by FACS analysis. Briefly, splenocytes were isolated by homogenizing the spleens between two glass slides in C-RPMI medium. Tumors were minced using a sterile razor blade and digested with a buffer containing DNase (12 U/ml), and collagenase (2 mg/ml) in PBS. After 60 min incubation at RT with agitation, cells were separated by vigorous pipetting. Red blood cells were lysed by RBC lysis buffer followed by several washes with complete RPMI-1640 medium containing 10% FBS. After filtration through a nylon mesh, tumor cells and splenocytes were resuspended in FACS buffer (2% FBS/PBS) and stained with anti-CD3-PerCP-Cy5.5, CD4-FITC, CD25-APC antibodies followed by permeabilization and staining with anti-Foxp3-PE. Flow cytometry analysis was performed using 4-color FACS calibur (BD) and data were analyzed using cell quest software (BD).

### Statistical Analysis

The log-rank Chi-Squared test was used for survival data and student's t-test for the CTL and ELISA assays, which were done in triplicates. A p-value of less than 0.05 (marked as *) was considered statistically significant in these analyzes. All statistical analysis was done with either Prism software, V.4.0a (2006) or SPSS software, V.15.0 (2006). For all FVB/N rat Her2/neu transgenic studies we used 8-14 mice per group, for all wild-type FVB/N studies we used at least 8 mice per group unless otherwise stated. All studies were repeated at least once except for the long term tumor study in Her2/neu transgenic mouse model.

### Example 15: Generation of L. monocytogenes Strains that Secrete LLO Fragments Fused to Her-2 Fragments: Construction of ADXS31-164

Construction of the chimeric Her2/neu gene (ChHer2) was as follows. Briefly, ChHer2 gene was generated by direct fusion of two extracellular (aa 40-170 and aa 359-433) and one intracellular fragment (aa 678-808) of the Her2/neu protein by SOEing PCR method. The chimeric protein harbors most of the known human MHC class I epitopes of the protein. ChHer2 gene was excised from the plasmid, pAdv138 (which was used to construct Lm-LLO-ChHer2) and cloned into LmddA shuttle plasmid, resulting in the plasmid pAdv164 (FIG. 20A). There are two major differences between these two plasmid backbones. 1) Whereas pAdv138 uses the chloramphenicol resistance marker (cat) for in vitro selection of recombinant bacteria, pAdv164 harbors the D-alanine racemase gene (dal) from Bacillus subtilis, which uses a metabolic complementation pathway for in vitro selection and in vivo plasmid retention in LmddA strain which lacks the dal-dat genes. This immunotherapy platform was designed and developed to address FDA concerns about the antibiotic resistance of the engineered Listeria immunotherapy strains. 2) Unlike pAdv138, pAdv164 does not harbor a copy of the prfA gene in the plasmid (see sequence below and FIG. 20A), as this is not necessary for in vivo complementation of the Lmdd strain. The LmddA immunotherapy strain also lacks the actA gene (responsible for the intracellular movement and cell-to-cell spread of Listeria) so the recombinant immunotherapy strains derived from this backbone are 100 times less virulent than those derived from the Lmdd, its parent strain. LmddA-based immunotherapies are also cleared much faster (in less than 48 hours) than the Lmdd-based immunotherapies from the spleens of the immunized mice. The expression and secretion of the fusion protein tLLO-ChHer2 from this strain was comparable to that of the Lm-LLO-ChHer2 in TCA precipitated cell culture supernatants after 8 hours of in vitro growth (FIG. 20B) as a band of ˜104 KD was detected by an anti-LLO antibody using Western Blot analysis. The Listeria backbone strain expressing only tLLO was used as negative control.

pAdv164 sequence (7075 base pairs) (see FIGS. 20A and 20B):

(SEQ ID NO: 87)
cggagtgtatactggcttactatgaggcactgatgagggtgtcagtgaa
gtgcttcatgtggcaggagaaaaaaggctgcaccggtgcgtcagcagaa
tatgtgatacaggatatattccgcacctcgctcactgactcgctacgct
cggtcgttcgactgcggcgagcggaaatggcttacgaacggggcggaga
tacctggaagatgccaggaagatacttaacagggaagtgagagggccgc
ggcaaagccgtattccataggctccgcccccctgacaagcatcacgaaa
tctgacgctcaaatcagtggtggcgaaacccgacaggactataaagata
ccaggcgtttccccctggcggctccctcgtgcgctctcctgttcctgcc
tttcggtttaccggtgtcattccgctgttatggccgcgtttgtctcatt
ccacgcctgacactcagaccgggtaggcagttcgctccaagctggactg
tatgcacgaaccccccgttcagtccgaccgctgcgccttatccggtaac
tatcgtcttgagtccaacccggaaagacatgcaaaagcaccactggcag
cagccactggtaattgatttagaggagttagtcttgaagtcatgcgccg
gttaaggctaaactgaaaggacaagattggtgactgcgctcctccaagc
cagttacctcggacaaagagaggtagctcagagaaccacgaaaaaccgc
cctgcaaggcggattacgattcagagcaagagattacgcgcagaccaaa
acgatctcaagaagatcatcttattaatcagataaaatatactagccct
cattgattagtatattcctatcttaaagttactatatgtggaggcatta
acatagttaatgacgtcaaaaggatagcaagactagaataaagctataa
agcaagcatataatattgcgatcatattagaagcgaatttcgccaatat
tataattatcaaaagagaggggtggcaaacggtataggcattattaggt
taaaaaatgtagaaggagagtgaaacccatgaaaaaaataatgctagta
ttattacacttatattagttagtctaccaattgcgcaacaaactgaagc
aaaggatgcatctgcattcaataaagaaaattcaatttcatccatggca
ccaccagcatctccgcctgcaagtcctaagacgccaatcgaaaagaaac
acgcggatgaaatcgataagtatatacaaggattggattacaataaaaa
caatgtattagtataccacggagatgcagtgacaaatgtgccgccaaga
aaaggttacaaagatggaaatgaatatattgagtggagaaaaagaagaa
atccatcaatcaaaataatgcagacattcaagagtgaatgcaatttcga
gcctaacctatccaggtgctctcgtaaaagcgaattcggaattagtaga
aaatcaaccagatgactccctgtaaaacgtgattcattaacactcagca
ttgatttgccaggtatgactaatcaagacaataaaatagagtaaaaaat
gccactaaatcaaacgttaacaacgcagtaaatacattagtggaaagat
ggaatgaaaaatatgctcaagcttatccaaatgtaagtgcaaaaattga
ttatgatgacgaaatggcttacagtgaatcacaattaattgcgaaatag
gtacagcatttaaagctgtaaataatagcttgaatgtaaacttcggcgc
aatcagtgaagggaaaatgcaagaagaagtcattagattaaacaaattt
actataacgtgaatgttaatgaacctacaagaccaccagattatcggca
aagctgaactaaagagcagttgcaagcgcttggagtgaatgcagaaaat
cctcctgcatatatctcaagtgtggcgtatggccgtcaagtttatttga
aattatcaactaattcccatagtactaaagtaaaagctgatttgatgct
gccgtaagcggaaaatctgtctcaggtgatgtagaactaacaaatatca
tcaaaaattatcatcaaagccgtaatttacggaggaccgcaaaagatga
agttcaaatcatcgacggcaacctcggagacttacgcgatattagaaaa
aaggcgctactataatcgagaaacaccaggagacccattgcttatacaa
caaacttcctaaaagacaatgaattagctgttattaaaaacaactcaga
atatattgaaacaacttcaaaagcttatacagatggaaaaattaacatc
gatcactctggaggatacgagctcaattcaacatacttgggatgaagta
aattatgatctcgagacccacctggacatgctccgccacctctaccagg
gctgccaggtggtgcagggaaacctggaactcacctacctgcccaccaa
tgccagcctgtccacctgcaggatatccaggaggtgcagggctacgtgc
tcatcgctcacaaccaagtgaggcaggtcccactgcagaggctgcggat
tgtgcgaggcacccagctattgaggacaactatgccctggccgtgctag
acaatggagacccgctgaacaataccacccctgtcacaggggcctcccc
aggaggcctgcgggagctgcagcttcgaagcctcacagagatcttgaaa
ggaggggtcttgatccagcggaacccccagctctgctaccaggacacga
ttagtggaagaatatccaggagtagctggctgcaagaagatctagggag
cctggcatactgccggagagattgatggggacccagcctccaacactgc
cccgctccagccagagcagctccaagtgatgagactctggaagagatca
caggttacctatacatctcagcatggccggacagcctgcctgacctcag
cgtatccagaacctgcaagtaatccggggacgaattctgcacaatggcg
cctactcgctgaccctgcaagggctgggcatcagctggctggggctgcg
ctcactgagggaactgggcagtggactggccctcatccaccataacacc
cacctctgcttcgtgcacacggtgccctgggaccagctattcggaaccc
gcaccaagctctgctccacactgccaaccggccagaggacgagtgtgtg
ggcgagggcctggcctgccaccagctgtgcgcccgagggcagcagaaga
tccggaagtacacgatgcggagactgctgcaggaaacggagctggtgga
gccgctgacacctagcggagcgatgcccaaccaggcgcagatgcggatc
ctgaaagagacggagctgaggaaggtgaaggtgatggatctggcgctta
ggcacagtctacaagggcatctggatccctgatggggagaatgtgaaaa
ttccagtggccatcaaagtgagagggaaaacacatcccccaaagccaac
aaagaaatcttagacgaagcatacgtgatggctggtgtgggctccccat
atgtctcccgccactgggcatctgcctgacatccacggtgcagctggtg
acacagcttatgccctatggctgcctcttagactaatctagacccgggc
cactaactcaacgctagtagtggatttaatcccaaatgagccaacagaa
ccagaaccagaaacagaacaagtaacattggagttagaaatggaagaag
aaaaaagcaatgatttcgtgtgaataatgcacgaaatcattgcttatta
tttaaaaagcgatatactagatataacgaaacaacgaactgaataaaga
atacaaaaaaagagccacgaccagttaaagcctgagaaactttaactgc
gagccttaattgattaccaccaatcaattaaagaagtcgagacccaaaa
taggtaaagtatttaattactttattaatcagatacttaaatatctgta
aacccattatatcgggtattgaggggatttcaagtattaagaagatacc
aggcaatcaattaagaaaaacttagttgattgccattagagtgattcaa
ctagatcgtagatctaactaattaattacgtaagaaaggagaacagctg
aatgaatatccatttgagtagaaactgtgcttcatgacggcagttaaag
tacaaatttaaaaatagtaaaattcgctcaatcactaccaagccaggta
aaagtaaaggggctatattgcgtatcgctcaaaaaaaagcatgattggc
ggacgtggcgttgactgacttccgaagaagcgattcacgaaaatcaaga
tacatttacgcattggacaccaaacgatatcgttatggtacgtatgcag
acgaaaaccgttcatacactaaaggacattctgaaaacaatttaagaca
aatcaataccttcatattgattagatattcacacggaaaaagaaactat
ttcagcaagcgatatataacaacagctattgatttaggattatgcctac
gttaattatcaaatctgataaaggttatcaagcatattagattagaaac
gccagtctatgtgacttcaaaatcagaatttaaatctgtcaaagcagcc
aaaataatctcgcaaaatatccgagaatattaggaaagtattgccagtt
gatctaacgtgcaatcattagggattgctcgtataccaagaacggacaa
tgtagaattattgatcccaattaccgttattattcaaagaatggcaaga
ttggtattcaaacaaacagataataagggattactcgttcaagtctaac
ggattaagcggtacagaaggcaaaaaacaagtagatgaaccctggataa
tctcttattgcacgaaacgaaattacaggagaaaagggatagtagggcg
caatagcgttatgataccctctattagcctactttagttcaggctattc
aatcgaaacgtgcgaatataatatgatgagataataatcgattagatca
acccttagaagaaaaagaagtaatcaaaattgttagaagtgcctattca
gaaaactatcaaggggctaatagggaatacattaccattattgcaaagc
agggtatcaagtgatttaaccagtaaagatttatagtccgtcaagggtg
gataaattcaagaaaaaaagaagcgaacgtcaacgtgacatagtcagaa
tggaaagaagatttaatggcttatattagcgaaaaaagcgatgtataca
agccttatttagcgacgaccaaaaaagagattagagaagtgctaggcat
tcctgaacggacattagataaattgctgaaggtactgaaggcgaatcag
gaaattactttaagattaaaccaggaagaaatggtggcattcaacttgc
tagtgttaaatcattgagctatcgatcattaaattaaaaaaagaagaac
gagaaagctatataaaggcgctgacagcttcgataatttagaacgtaca
tttattcaagaaactctaaacaaattggcagaacgccccaaaacggacc
cacaactcgatttgatagctacgatacaggctgaaaataaaacccgcac
tatgccattacatttatatctatgatacgtgatgatactagctggctag
cttaattgcttatatttacctgcaataaaggatacttacttccattata
ctcccattaccaaaaacatacggggaacacgggaacttattgtacaggc
cacctcatagttaatggatcgagccacctgcaatctcatccatggaaat
atattcatccccctgccggcctattaatgtgacttagtgcccggcggat
attcctgatccagctccaccataaattggtccatgcaaattcggccggc
aattacaggcgattccatcacaaggatgtcggtccattcaattacggag
ccagccgtccgcatagcctacaggcaccgtcccgatccatgtgtattac
cgctgtgtactcggctccgtagctgacgctctcgccattctgatcagat
gacatgtgacagtgtcgaatgcagggtaaatgccggacgcagctgaaac
ggtatctcgtccgacatgtcagcagacgggcgaaggccatacatgccga
tgccgaatctgactgcattaaaaaagccattacagccggagtccagcgg
cgctgacgcgcagtggaccattagattattaacggcagcggagcaatca
gctattaaagcgctcaaactgcattaagaaatagcctctactattcatc
cgctgtcgcaaaatgggtaaatacccctagcactttaaacgagggagcg
gtcaagaattgccatcacgactgaacttcacctctgatttacaccaagt
ctgacatccccgtatcgaccacagatgaaaatgaagagaaccattacgt
gtggcgggctgcctcctgaagccattcaacagaataacctgttaaggtc
acgtcatactcagcagcgattgccacatactccgggggaaccgcgccaa
gcaccaatataggcgcatcaatccattagcgcagtgaaatcgcttcatc
caaaatggccacggccaagcatgaagcacctgcgtcaagagcagcattg
ctgatctgcatcaccatgcccgtaggcgtagcatcacaactgccatcaa
gtggacatgacaccgatatgattacatattgctgacattaccatatcgc
ggacaagtcaataccgcccacgtatctctgtaaaaaggattgtgctcat
ggaaaactcctctattatcagaaaatcccagtacgtaattaagtatttg
agaattaatatatattgattaatactaagatacccagattcacctaaaa
aacaaatgatgagataatagctccaaaggctaaagaggactataccaac
tatttgaaattaa

### Example 16: ADXS31-164 is as Immunogenic as Lm-LLO-ChHER2

Immunogenic properties of ADXS31-164 in generating anti-Her2/neu specific cytotoxic T cells were compared to those of the Lm-LLO-ChHer2 immunotherapy in a standard CTL assay. Both immunotherapies elicited strong but comparable cytotoxic T cell responses toward Her2/neu antigen expressed by 3T3/neu target cells. Accordingly, mice immunized with a Listeria expressing only an intracellular fragment of Her2-fused to LLO showed lower lytic activity than the chimeras which contain more MHC class I epitopes. No CTL activity was detected in naïve animals or mice injected with the irrelevant Listeria immunotherapy (FIG. 21A). ADXS31-164 was also able to stimulate the secretion of IFN-γ by the splenocytes from wild type FVB/N mice (FIG. 21B). This was detected in the culture supernatants of these cells that were co-cultured with mitomycin C treated NT-2 cells, which express high levels of Her2/neu antigen (FIG. 21C).

Proper processing and presentation of the human MHC class I epitopes after immunizations with ADXS31-164 was tested in HLA-A2 mice. Splenocytes from immunized HLA-A2 transgenics were co-incubated for 72 hours with peptides corresponding to mapped HLA-A2 restricted epitopes located at the extracellular (HLYQGCQVV SEQ ID NO: 59 or KIFGSLAFL SEQ ID NO: 60) or intracellular (RLLQETELV SEQ ID NO: 61) domains of the Her2/neu molecule (FIG. 21C). A recombinant ChHer2 protein was used as positive control and an irrelevant peptide or no peptide as negative controls. The data from this experiment show that ADXS31-164 is able to elicit anti-Her2/neu specific immune responses to human epitopes that are located at different domains of the targeted antigen.

### Example 17: ADXS31-164 was More Efficacious than Lm-LLO-ChHER2 in Preventing the Onset of Spontaneous Mammary Tumors

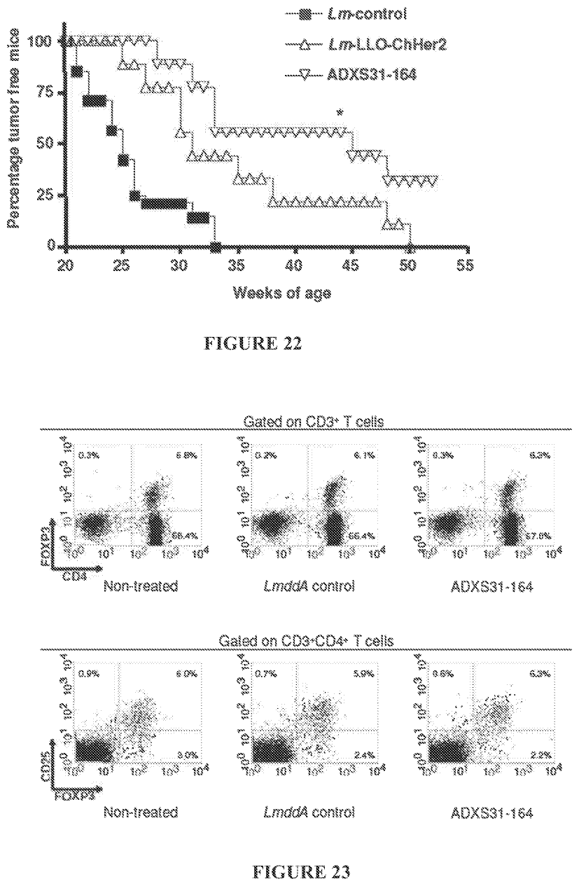

Anti-tumor effects of ADXS31-164 were compared to those of Lm-LLO-ChHer2 in Her2/neu transgenic animals which develop slow growing, spontaneous mammary tumors at 20-25 weeks of age. All animals immunized with the irrelevant Listeria-control immunotherapy developed breast tumors within weeks 21-25 and were sacrificed before week 33. In contrast, Listeria-Her2/neu recombinant immunotherapies caused a significant delay in the formation of the mammary tumors. On week 45, more than 50% of ADXS31-164 vaccinated mice (5 out of 9) were still tumor free, as compared to 25% of mice immunized with Lm-LLO-ChHer2. At week 52, 2 out of 8 mice immunized with ADXS31-164 still remained tumor free, whereas all mice from other experimental groups had already succumbed to their disease (FIG. 22). These results indicate that despite being more attenuated, ADXS31-164 is more efficacious than Lm-LLO-ChHer2 in preventing the onset of spontaneous mammary tumors in Her2/neu transgenic animals.

### Example 18: Mutations in HER2/Neu Gene Upon Immunization with ADXS31-164

Mutations in the MHC class I epitopes of Her2/neu have been considered responsible for tumor escape upon immunization with small fragment immunotherapies or trastuzumab (Herceptin), a monoclonal antibody that targets an epitope in the extracellular domain of Her2/neu. To assess this, genomic material was extracted from the escaped tumors in the transgenic animals and sequenced the corresponding fragments of the neu gene in tumors immunized with the chimeric or control immunotherapies. Mutations were not observed within the Her-2/neu gene of any vaccinated tumor samples suggesting alternative escape mechanisms (data not shown).

### Example 19: ADXS31-164 Causes a Significant Decrease in Intra-Tumoral T Regulatory Cells

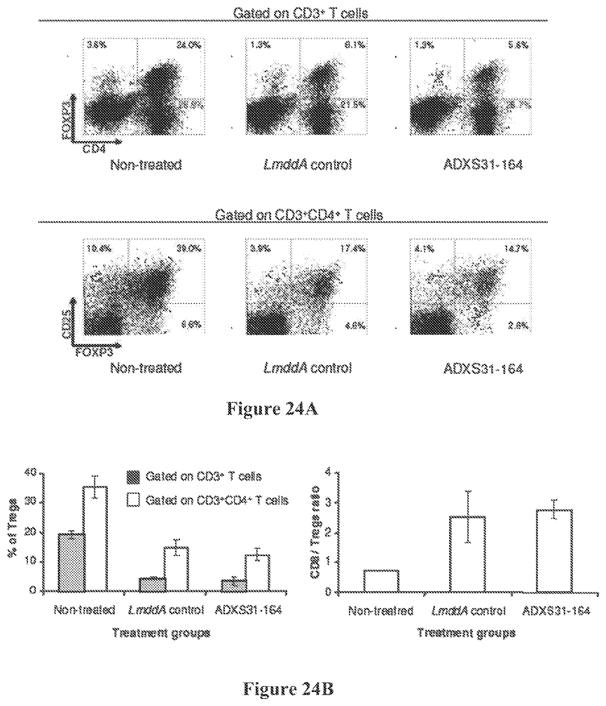

To elucidate the effect of ADXS31-164 on the frequency of regulatory T cells in spleens and tumors, mice were implanted with NT-2 tumor cells. Splenocytes and intra-tumoral lymphocytes were isolated after three immunizations and stained for Tregs, which were defined as CD3⁺/CD4⁺/CD25⁺/FoxP3⁺ cells, although comparable results were obtained with either FoxP3 or CD25 markers when analyzed separately. The results indicated that immunization with ADXS31-164 had no effect on the frequency of Tregs in the spleens, as compared to an irrelevant Listeria immunotherapy or the naïve animals (FIG. 23). In contrast, immunization with the Listeria immunotherapies caused a considerable impact on the presence of Tregs in the tumors (FIG. 24A). Whereas in average 19.0% of all CD3+ T cells in untreated tumors were Tregs, this frequency was reduced to 4.2% for the irrelevant immunotherapy and 3.4% for ADXS31-164, a 5-fold reduction in the frequency of intra-tumoral Tregs (FIG. 24B). The decrease in the frequency of intra-tumoral Tregs in mice treated with either of the LmddA immunotherapies could not be attributed to differences in the sizes of the tumors. In a representative experiment, the tumors from mice immunized with ADXS31-164 were significantly smaller [mean diameter (mm)±SD, 6.71±0.43, n=5] than the tumors from untreated mice (8.69±0.98, n=5, p<0.01) or treated with the irrelevant immunotherapy (8.41±1.47, n=5, p=0.04), whereas comparison of these last two groups showed no statistically significant difference in tumor size (p=0.73). The lower frequency of Tregs in tumors treated with LmddA immunotherapies resulted in an increased intratumoral CD8/Tregs ratio, suggesting that a more favorable tumor microenvironment can be obtained after immunization with LmddA immunotherapies. However, only the immunotherapy expressing the target antigen HER2/neu (ADXS31-164) was able to reduce tumor growth, indicating that the decrease in Tregs has an effect only in the presence on antigen-specific responses in the tumor.

### Example 20: Peripheral Immunization with ADXS31-164 can Delay the Growth of a Metastatic Breast Cancer Cell Line in the Brain

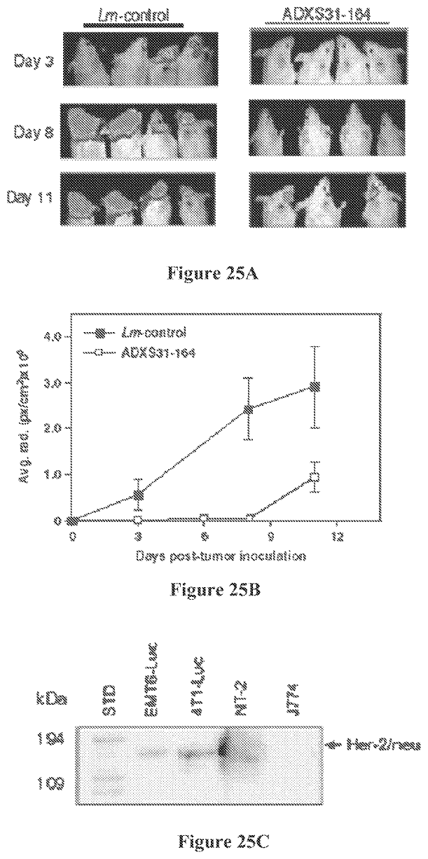

Mice were immunized IP with ADXS31-164 or irrelevant Lm-control immunotherapies and then implanted intra-cranially with 5,000 EMT6-Luc tumor cells, expressing luciferase and low levels of Her2/neu (FIG. 25A). Tumors were monitored at different times post-inoculation by ex vivo imaging of anesthetized mice. On day 8 post-tumor inoculation tumors were detected in all control animals, but none of the mice in ADXS31-164 group showed any detectable tumors (FIGS. 25A and 25B). ADXS31-164 could clearly delay the onset of these tumors, as on day 11 post-tumor inoculation all mice in negative control group had already succumbed to their tumors, but all mice in ADXS31-164 group were still alive and only showed small signs of tumor growth. These results strongly suggest that the immune responses obtained with the peripheral administration of ADXS31-164 could possibly reach the central nervous system and that LmddA-based immunotherapies might have a potential use for treatment of CNS tumors.

### Example 21: Peptide “Minigene” Expression System

### Materials and Methods

This expression system is designed to facilitate cloning of panels of recombinant proteins containing distinct peptide moieties at the carboxy-terminus. This is accomplished by a simple PCR reaction utilizing a sequence encoding one of the SS-Ub-Peptide constructs as a template. By using a primer that extends into the carboxy-terminal region of the Ub sequence and introducing codons for the desired peptide sequence at the 3′ end of the primer, a new SS-Ub-Peptide sequence can be generated in a single PCR reaction. The 5′ primer encoding the bacterial promoter and first few nucleotides of the ActA signal sequence is the same for all constructs. The constructs generated using this strategy are represented schematically in FIGS. 26A-26C. In this example, two constructs are described. One contains a model peptide antigen presented on mouse MHC class I and the second construct indicates where a therapeutically relevant peptide, such as one derived from a human glioblastoma (GBM) TAA, would be substituted. For clarity, we have designated the constructs diagramed in FIGS. 26A-C as containing an ActA₁₋₁₀₀ secretion signal. However, an LLO based secretion signal could be substituted with equal effect.

One of the advantages of the proposed system is that it will be possible to load cells with multiple peptides using a single Listeria vector construct. Multiple peptides will be introduce into recombinant attenuated Listeria (e.g. prfA mutant Listeria or a dal/dat/actA mutant Listeria) using a modification of the single peptide expression system described above. A chimeric protein encoding multiple distinct peptides from sequential SS-Ub-Peptide sequences encoded in one insert. Shine-Dalgarno ribosome binding sites are introduced before each SS-Ub-Peptide coding sequence to enable separate translation of each of the peptide constructs. FIG. 26C demonstrates a schematic representation of a construct designed to express 4 separate peptide antigens from one strain of recombinant Listeria. Since this is strictly a representation of the general expression strategy, we have included 4 distinct MHC class I binding peptides derived from known mouse or human tumor associated- or infectious disease antigens.

### Materials & Methods (Examples 22-24)

Plasmid pAdv142 and strain LmddA142 have been described above at Example 7.

Additional details are provided below.

Construction of Plasmid pAdv142 and Strain LmddA142

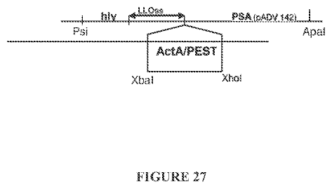

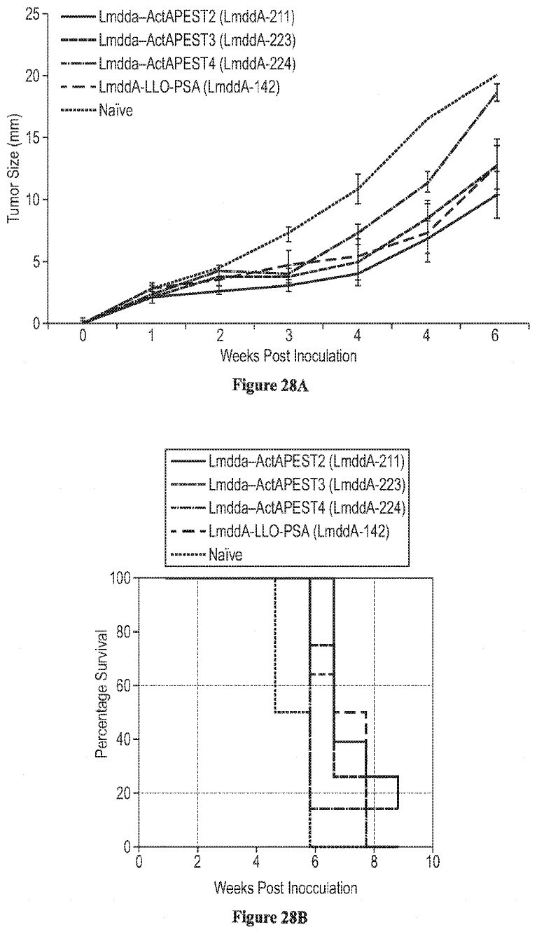

This plasmid is next generation of the antibiotic free plasmid, pTV3 that was previously constructed by Verch et al. The unnecessary copy of the virulence gene transcription activator, prfA was deleted from plasmid pTV3 since Lm-ddA contains a copy of prfA gene in the chromosome. Therefore, the presence of prfA gene in the dal containing plasmid was not essential. Additionally, the cassette for p60-Listeria dal at the NheI/PacI restriction site was replaced by p60-Bacillus subtilis dal (dal_{Bs}) resulting in the plasmid pAdv134. Further, pAdv134 was restricted with XhoI/XmaI to clone human PSA, klk3 resulting in the plasmid, pAdv142. The new plasmid pAdv 142 (FIG. 11C) contains dal_{Bs} and its expression was under the control of Lm p60 promoter. The shuttle plasmid pAdv142 could complement the growth of both E. coli ala drx MB2159 as well as Lmdd in the absence of exogenous addition of D-alanine. The antigen expression cassette in the plasmid pAdv 142 consists of hly promoter and tLLO-PSA fusion protein (FIG. 27).

The plasmid pAdv142 was transformed to the Listeria background strain, LmddA resulting in LmddA142 or ADXS31-142. The expression and secretion of LLO-PSA fusion protein by the strain, ADXS31-142 was confirmed by western analysis using anti-LLO and anti-PSA antibody and is shown in FIG. 11D. There was stable expression and secretion of LLO-PSA fusion protein by the strain, ADXS31-142 after two in vivo passages in C57BL/6 mice.

### Construction of LmddA211, LmddA223 and LmddA224 Strains

The different ActA/PEST regions were cloned in the plasmid pAdv142 to create the three different plasmids pAdv211, pAdv223 and pAdv224 containing different truncated fragments of ActA protein.

LLO Signal Sequence (LLOss)-ActAPEST2 (pAdv211)/LmddA211

First two fragments PsiI-LLOss-XbaI (817 bp in size) and LLOss-XbaI-ActA-PEST2 (602 bp in size) were amplified and then fused together by using SOEing PCR method with an overlap of 25 bases. This PCR product now contains PsiI-LLOss-XbaI-ActAPEST2-XhoI a fragment of 762 bp in size. The new PsiI-LLOss-XbaI-ActAPEST2-XhoI PCR product and pAdv142 (LmddA-PSA) plasmid were digested with PsiI/XhoI restriction enzymes and purified. Ligation was set up and transformed into MB2159 electro competent cells and plated onto LB agar plates. The PsiI-LLOss-XbaI-ActAPEST2/pAdv 142 (PSA) clones were selected and screened by insert-specific PCR reaction PsiI-LLOss-XbaI-ActAPEST2/pAdv 142 (PSA) clones #9, 10 were positive and the plasmid purified by mini preparation. Following screening of the clones by PCR screen, the inserts from positive clones were sequenced. The plasmid PsiI-LLOss-XbaI-ActAPEST2/pAdv 142 (PSA) referred as pAdv211.10 was transformed into Listeria LmddA mutant electro competent cells and plated onto BHI/strep agar plates. The resulting LmddA211 strain was screened by colony PCR. Several Listeria colonies were selected and screened for the expression and secretion of endogenous LLO and ActAPEST2-PSA (LA229-PSA) proteins. There was stable expression of ActAPEST2-PSA fusion proteins after two in vivo passages in mice.

### LLOss-ActAPEST3 and PEST4:

ActAPEST3 and ActAPEST4 fragments were created by PCR method. PCR products containing LLOss-XbaI-ActAPEST3-XhoI (839 bp in size) and LLOss-XbaI-ActAPEST4-XhoI a fragments (1146 bp in size) were cloned in pAdv142. The resulting plasmid pAdv223 (PsiI-LLOss-XbaI-ActAPEST3-XhoI/pAdv 142) and pAdv224 (PsiI-LLOss-XbaI-ActAPEST4/pAdv 142) clones were selected and screened by insert-specific PCR reaction. The plasmids pAdv223 and pAdv224 were transformed to the LmddA backbone resulting in LmddA223 and LmddA224, respectively. Several Listeria colonies were selected and screened for the expression and secretion of endogenous LLO, ActAPEST3-PSA (LmddA223) or ActAPEST4-PSA (LmddA224) proteins. There was stable expression and secretion of the fusion protein ActAPEST3-PSA (LmddA223) or ActAPEST4-PSA (LmddA224) after two in vivo passages in mice.

### Experimental Plan 1

The therapeutic efficacy of the ActA-PEST-PSA (PEST3, PEST2 and PEST4 sequences) and tLLO-PSA using TPSA23 (PSA expressing tumor model) were evaluated and compared. Untreated mice were used as control group. In parallel evaluated the immune responses were also using intracellular cytokine staining for interferon-gamma and PSA tetramer staining.

### For the Tumor Regression Study.

Ten groups of eight C57BL/6 mice (7 weeks old males) were implanted subcutaneously with 1×10⁶ of TPSA23 cells on day 0. On Day 6 they received immunization which was followed by 2 booster doses which were 1 week apart. Tumor growth was monitored every week until they reached a size of 1.2 cm in average diameter.

### Immunogenicity Study.

2 groups of C57BL/6 mice (7 weeks old males) were immunized 3 times with one week interval with the immunotherapies listed in the table below. Six days after the last boost injection, mice were sacrificed, and the spleens will be harvested and the immune responses were tested for tetramer staining and IFN-γ secretion by intracellular cytokine staining

### Experimental Plan 2

This experiment was a repeat of Experimental plan 1, however, the Naïve, tLLO, ActA/PEST2-PSA and tLLO-PSA groups were only included Similar to Experimental plan 1, the therapeutic efficacy was evaluated using TPSA23 (PSA expressing tumor model). Five C57BL/6 mice per group were implanted subcutaneously with 1×10⁶ of TPSA23 cells on day 0. On Day 6 they received immunization (1×10⁸ CFU/mL) which was followed by booster 1 week later. Spleen and tumor was collected on day 6 post last treatment. The immune response was monitored using PSA pentamer staining in both spleen and tumor.

### Materials & Methods:

TPSA23 cells are cultured in complete medium. Two days prior to implanting tumor cells in mice, TPSA23 cells were sub-cultured in complete media. On the day of the experiment (Day 0), cells were trypsinized and washed twice with PBS. Cells were counted and re-suspended at a concentration of 1×10⁶ cells/200 ul in PBS/mouse for injection. Tumor cells were injected subcutaneously in the flank of each mouse.

### Complete Medium for TPSA23 Cells

Complete medium for TPSA23 cells was prepared by mixing 430 ml of DMEM with Glucose, 45 ml of fetal calf serum (FCS), 25 ml of Nu-Serum IV, 5 ml 100× L-Glutamine, of 100 mM Na-Pyruvate, 5 ml of 10,000 U/mL Penicillin/Streptomycin. 0.005 mg/ml of Bovine Insulin and 10 nM of Dehydroisoandrosterone was added to the flask while splitting cells.

Complete Medium for Splenocytes (c-RPMI)

Complete medium was prepared by mixing 450 ml of RPMI 1640, 50 ml of fetal calf serum (FCS), 5 ml of 1M HEPES, 5 ml of 100× Non-essential amino acids (NEAA), 5 ml of 100× L-Glutamine, 5 ml of 100 mM Na-Pyruvate, 5 ml of 10,000 U/mL Penicillin/Streptomycin and 129 ul of 14.6M 2-Mercaptoethanol.

### Preparing Isolated Splenocytes

Work was performed in biohazard hood. Spleens were harvested from experimental and control mice groups using sterile forceps and scissors. They were transport in 15 ml tubes containing 10 ml PBS to the lab. Spleen from each mouse was processed separately. Spleen was taken in a sterile Petri dish and mashed using the back of plunger from a 3 mL syringe. Spleen cells were transferred to a 15 ml tube containing 10 ml of RPMI 1640. Cells were pelleted by centrifugation at 1,000 RPM for 5 min at 4° C. The supernatant was discarded in 10% bleach. Cell pellet was gently broken by tapping. RBC was lysed by adding 2 ml of RBC lysis buffer per spleen to the cell pellet. RBC lysis was allowed for 2 min. Immediately, 10 ml of c-RPMI medium was added to the cell suspension to deactivate RBC lysis buffer. Cells were pelleted by centrifugation at 1,000 RPM for 5 min at 4° C. The supernatant was discarded and cell pellet was re-suspended in 10 ml of c-RPMI and passed through a cell strainer. Cells were counted using hemocytometer and the viability was checked by mixing 10 ul of cell suspension with 90 ul of Trypan blue stain. About 2×10⁶ cells were used for pentamer staining. (Note: each spleen should yield 1-2×10⁸ cells).

Preparing Single Cell Suspension from Tumors Using Miltenyi Mouse Tumor Dissociation Kit

Enzyme mix was prepared by adding 2.35 mL of RPMI 1640, 100 μL of Enzyme D, 50 μL of Enzyme R, and 12.5 μL of Enzyme A into a gentleMACS C Tube. Tumor (0.04-1 g) was cut into small pieces of 2-4 mm and transferred into the gentleMACS C Tube containing the enzyme mix. The tube was attached upside down onto the sleeve of the gentle MACS Dissociator and the Program m_impTumor_02 was run. After termination of the program, C Tube was detached from the gentle MACS Dissociator. The sample was incubated for 40 minutes at 37° C. with continuous rotation using the MACSmix Tube Rotator. After completion of incubation the C tube was again attached upside down onto the sleeve of the gentle MACS Dissociator and the program m_impTumor_03 was run twice. The cell suspension was filtered through 70 μm filter placed on a 15 mL tube. The filter was also washed with 10 mL of RPMI 1640. The cells were centrifuged at 300×g for 7 minutes. The supernatant was discarded and the cells were re-suspended in 10 ml of RPMI 1640. At this point one can divide the cells for pentamer staining.

### Pentamer Staining of Splenocytes

The PSA-specific T cells were detected using commercially available PSA-H-2D^{b} pentamer from ProImmune using manufacturers recommended protocol. Splenocytes were stained for CD8, CD62L, CD3 and Pentamer. While tumor cells were stained for CD8, CD62L, CD45 and Pentamer. The CD3⁺CD8⁺ CD62L^{low} cells were gated to determine the frequency of CD3⁺CD8⁺ CD62L^{low} PSA pentamer⁺ cells. The stained cells were acquired and analyzed on FACS Calibur using Cell quest software.

### Materials Needed for Pentamer Staining

Splenocytes (preparation described above), Pro5® Recombinant MHC PSA Pentamer conjugated to PE. (Note: Ensure that the stock Pentamer is stored consistently at 4° C. in the dark, with the lid tightly closed), anti-CD3 antibody conjugated to PerCP Cy5.5, anti-CD8 antibody conjugated to FITC and anti-CD62L antibody conjugated to APC, wash buffer (0.1% BSA in PBS) and fix solution (1% heat inactivated fetal calf serum (HI-FCBS), 2.5% formaldehyde in PBS)

### Standard Staining Protocol

Pro5® PSA Pentamer was centrifuged in a chilled microcentrifuge at 14,000×g for 5-10 minutes to remove any protein aggregates present in the solution. These aggregates may contribute to non-specific staining if included in test volume. 2×10⁶ splenocytes were allocated per staining condition and 1 ml of wash buffer was added per tube. Cells were centrifuged at 500×g for 5 min in a chilled centrifuge at 4° C. The cell pellet was re-suspended in the residual volume (˜50 μl). All tubes were chilled on ice for all subsequent steps, except where otherwise indicated. 10 μl of labeled Pentamer was added to the cells and mixed by pipetting. The cells were incubated at room temperature (22° C.) for 10 minutes, shielded from light. Cells were washed with 2 ml of wash buffer per tube and re-suspend in residual liquid (˜50 μl). An optimal amount of anti-CD3, anti-CD8 and anti-CD62L antibodies were added (1:100 dilution) and mixed by pipetting. Single stain control samples were also made at this point. Samples were incubated on ice for 20 minutes, shielded from light. Cells were washed twice with 2 ml wash buffer per tube. The cell pellet was re-suspended in the residual volume (˜50 μl). 200 μl of fix solution was added to each tube and vortexed. The tubes were stored in dark in the refrigerator until ready for data acquisition. (Note: the morphology of the cell changes after fixing, so it is advisable to leave the samples for 3 hours before proceeding with data acquisition. Samples can be stored for up to 2 days).

### Intracellular Cytokine Staining (IFN-γ) Protocol:

2×10⁷ cells/ml splenocytes were taken in FACS tubes and 100 μl of Brefeldin A (BD Golgi Plug) was added to the tube. For stimulation, 2 μM Peptide was added to the tube and the cells were incubated at room temperature for 10-15 minutes. For positive control samples, PMA (10 ng/ml) (2×) and ionomycin (1 μg/ml) (2×) was added to corresponding tubes. 100 μl of medium from each treatment was added to the corresponding wells in a U-bottom 96-well plate. 100 μl of cells were added to the corresponding wells (200 μl final volume−medium+cells). The plate was centrifuged at 600 rpm for 2 minutes and incubated at 37° C. 5% CO₂ for 5 hours. Contents from the plate was transferred to FACS tubes. 1 ml of FACS buffer was added to each tube and centrifuged at 1200 rpm for 5 min. The supernatant was discarded. 200 μl of 2.4G2 supernatant and 10 μl of rabbit serum was added to the cells and incubated for 10 minutes at room temperature. The cells were washed with 1 mL of FACS buffer. The cells were collected by centrifugation at 1200 rpm for 5 minutes. Cells were suspended in 50 μl of FACS buffer containing the fluorochrome-conjugated monoclonal antibodies (CD8 FITC, CD3 PerCP-Cy5.5, CD62L APC) and incubated at 4° C. for 30 minutes in the dark. Cells were washed twice with 1 mL FACS buffer and re-suspended in 200 μl of 4% formalin solution and incubated at 4° C. for 20 min. The cells were washed twice with 1 mL FACS buffer and re-suspended in BD Perm/Wash (0.25 ml/tube) for 15 minutes. Cells were collected by centrifugation and re-suspended in 50 μl of BD Perm/Wash solution containing the fluorochrome-conjugated monoclonal antibody for the cytokine of interest (IFNg-PE). The cells were incubated at 4° C. for 30 minutes in the dark. Cells were washed twice using BD Perm/Wash (1 ml per tube) and re-suspended in 200 μl FACS buffer prior to analysis.

### Results

### Example 22: Vaccination with Recombinant Listeria Constructs Leads to Tumor Regression

The data showed that by week 1, all groups had developed tumor with the average size of 2-3 mm. On week 3 (Day 20) mice immunized with ActA/PEST2 (also known as “LA229”)-PSA, ActA/PEST3-PSA and ActA/PEST3-PSA and LmddA-142 (ADXS31-142), which expresses a tLLO fused to PSA showed, tumor regression and slow down of the tumor growth. By week 6, all mice in naïve and most in ActAPEST4-PSA treated group had big tumors and had to be euthanized (FIG. 28A). However, LmddA-142, ActA-PEST2 and ActA-PEST3 mice groups showed better tumor regression and survival rate (FIGS. 28A and 28B).

### Example 23: Vaccination with Recombinant Listeria Generates High Levels of Antigen-Specific T Cells

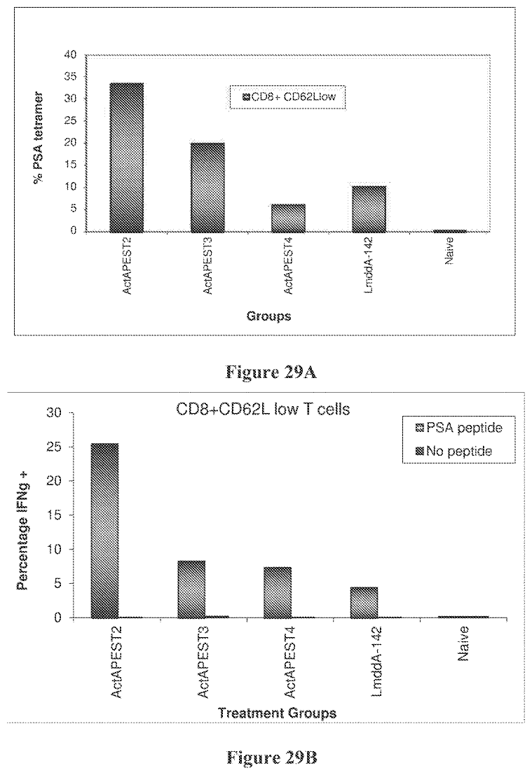

LmddA-ActAPEST2-PSA immunotherapy generated high levels of PSA-specific T cells response compared to LmddA-ActAPEST (3 or 4)-PSA, or LmddA-142 (FIG. 29A). The magnitude of PSA tetramer specific T cells in PSA-specific immunotherapies was 30 fold higher than naïve mice. Similarly, higher levels of IFN-γ secretion was observed for LmddA-ActAPEST2-PSA immunotherapy in response to stimulation with PSA-specific antigen (FIG. 29B).

### Example 24: Vaccination with Acta/Pest2 (La229) Generates a High Number of Antigen-Specific Cd8+ T Cells in Spleen

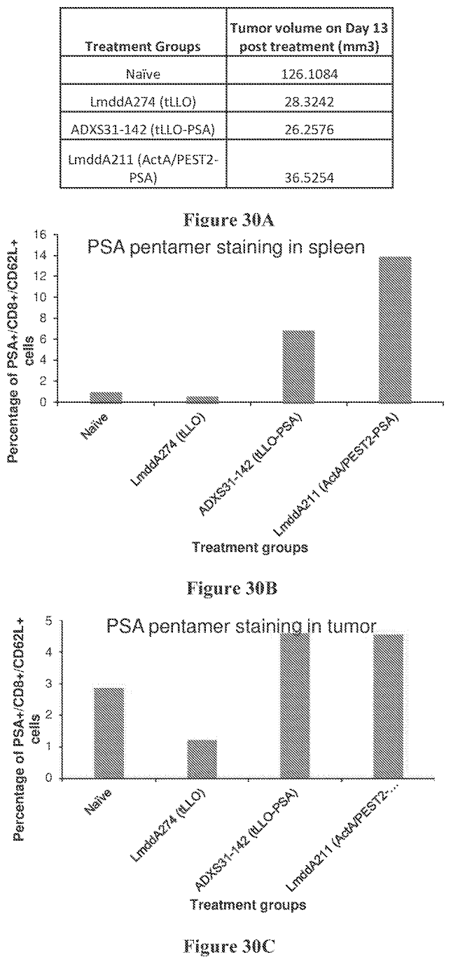

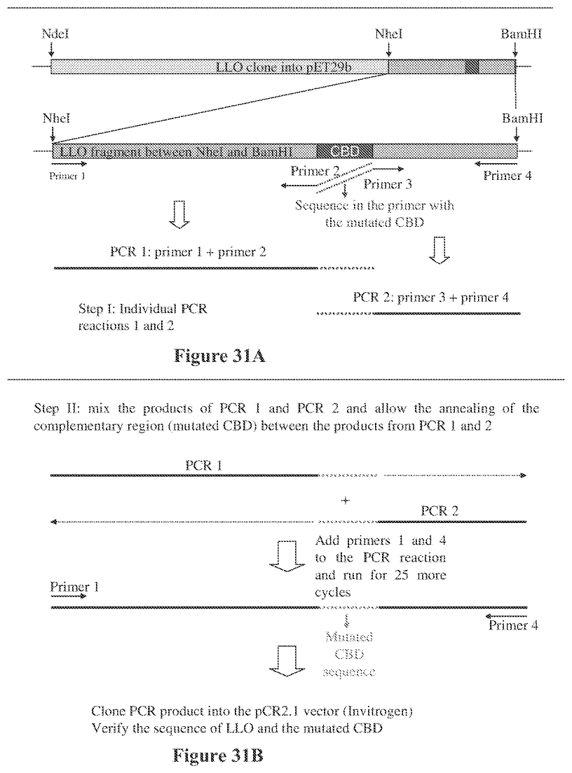

Lm expressing ActA/PEST2 fused PSA was able to generate higher numbers of PSA specific CD8+ T cells in spleen compared to Lm expressing tLLO fused PSA or tLLO treated group. The number of PSA specific CD8+ T cells infiltrating tumors were similar for both Lm-tLLO-PSA and Lm-ActA/PEST2-PSA immunized mice (FIGS. 30B and 30C). Also, tumor regression ability of Lm expressing ActA/PEST2-PSA was similar to that seen for LmddA-142 which expresses tLLO-PSA (FIG. 30A).

### Example 25: Site-Directed Mutagenesis of the LLO Cholesterol-Binding Domain

Site-directed mutagenesis was performed on LLO to introduce inactivating point mutations in the CBD, using the following strategy. The resulting protein is termed “mutLLO”:

Subcloning of LLO into pET29b

The amino acid sequence of wild-type LLO is:

(SEQ ID NO: 2)
MKKIMLVFITLILVSLPIAQQTEAKDASAFNKENSISSVAPPASPPASPK
TPIEKKHADEIDKYIQGLDYNKNNVLVYHGDAVTNVPPRKGYKDGNEYIV
VEKKKKSINQNNADIQVVNAISSLTYPGALVKANSELVENQPDVLPVKRD
SLTLSIDLPGMTNQDNKIVVKNATKSNVNNAVNTLVERWNEKYAQAYSNV
SAKIDYDDEMAYSESQLIAKFGTAFKAVNNSLNVNFGAISEGKMQEEVIS
FKQIYYNVNVNEPTRPSRFFGKAVTKEQLQALGVNAENPPAYISSVAYGR
QVYLKLSTNSHSTKVKAAFDAAVSGKSVSGDVELTNIIKNSSFKAVIYGG
SAKDEVQIIDGNLGDLRDILKKGATFNRETPGVPIAYTTNFLKDNELAVI
KNNSEYIETTSKAYTDGKINIDHSGGYVAQFNISWDEVNYDPEGNEIVQH
KNWSENNKSKLAHFTSSIYLPGNARNINVYAKE [symbol] TGLAWE [symbol] RTVIDDR
NLPLVKNRNISIWGTTLYPKYSNKVDNPIE.

The signal peptide and the cholesterol-binding domain (CBD) are underlined, with 3 critical residues in the CBD (C484, W491, and W492) in bold-italics.

A 6×His tag (HHHHHH) was added to the C-terminal region of LLO. The amino acid sequence of His-tagged LLO is:

(SEQ ID NO: 62)
MKKIMLVFITLILVSLPIAQQTEAKDASAFNKENSISSVAPPASPPASPK
TPIEKKHADEIDKYIQGLDYNKNNVLVYHGDAVTNVPPRKGYKDGNEYIV
VEKKKKSINQNNADIQVVNAISSLTYPGALVKANSELVENQPDVLPVKRD
SLTLSIDLPGMTNQDNKIVVKNATKSNVNNAVNTLVERWNEKYAQAYSNV
SAKIDYDDEMAYSESQLIAKFGTAFKAVNNSLNVNFGAISEGKMQEEVIS
FKQIYYNVNVNEPTRPSRFFGKAVTKEQLQALGVNAENPPAYISSVAYGR
QVYLKLSTNSHSTKVKAAFDAAVSGKSVSGDVELTNIIKNSSFKAVIYGG
SAKDEVQIIDGNLGDLRDILKKGATFNRETPGVPIAYTTNFLKDNELAVI
KNNSEYIETTSKAYTDGKINIDHSGGYVAQFNISWDEVNYDPEGNEIVQH
KNWSENNKSKLAHFTSSIYLPGNARNINVYAKE [symbol] TGLAWE [symbol] RTVIDDR
NLPLVKNRNISIWGTTLYPKYSNKVDNPIEHHHHHH.

A gene encoding a His-tagged LLO protein was digested with NdeI/BamHI, and the NdeI/BamHI was subcloned into the expression vector pET29b, between the NdeI and BamHI sites. The sequence of the gene encoding the LLO protein is:

(SEQ ID NO: 63)
catatgaaggatgcatctgcattcaataaagaaaattcaatttcatccgt
ggcaccaccagcatctccgcctgcaagtcctaagacgccaatcgaaaaga
aacacgcggatgaaatcgataagtatatacaaggattggattacaataaa
aacaatgtattagtataccacggagatgcagtgacaaatgtgccgccaag
aaaaggttacaaagatggaaatgaatatattgttgtggagaaaaagaaga
aatccatcaatcaaaataatgcagacattcaagttgtgaatgcaatttcg
agcctaacctatccaggtgctctcgtaaaagcgaattcggaattagtaga
aaatcaaccagatgttctccctgtaaaacgtgattcattaacactcagca
ttgatttgccaggtatgactaatcaagacaataaaatagttgtaaaaaat
gccactaaatcaaacgttaacaacgcagtaaatacattagtggaaagatg
gaatgaaaaatatgctcaagcttattcaaatgtaagtgcaaaaattgatt
atgatgacgaaatggcttacagtgaatcacaattaattgcgaaatttggt
acagcatttaaagctgtaaataatagcttgaatgtaaacttcggcgcaat
cagtgaagggaaaatgcaagaagaagtcattagttttaaacaaatttact
ataacgtgaatgttaatgaacctacaagaccttccagatttttcggcaaa
gctgttactaaagagcagttgcaagcgcttggagtgaatgcagaaaatcc
tcctgcatatatctcaagtgtggcgtatggccgtcaagtttatttgaaat
tatcaactaattcccatagtactaaagtaaaagctgcttttgatgctgcc
gtaagcggaaaatctgtctcaggtgatgtagaactaacaaatatcatcaa
aaattcttccttcaaagccgtaatttacggaggttccgcaaaagatgaag
ttcaaatcatcgacggcaacctcggagacttacgcgatattttgaaaaaa
ggcgctacttttaatcgagaaacaccaggagttcccattgcttatacaac
aaacttcctaaaagacaatgaattagctgttattaaaaacaactcagaat
atattgaaacaacttcaaaagcttatacagatggaaaaattaacatcgat
cactctggaggatacgttgctcaattcaacatttcttgggatgaagtaaa
ttatgatcctgaaggtaacgaaattgttcaacataaaaactggagcgaaa
acaataaaagcaagctagctcatttcacatcgtccatctatttgcctggt
aacgcgagaaatattaatgtttacgctaaagaa [symbol] actggtttagctt
gggaa [symbol] agaacggtaattgatgaccggaacttaccacttgtgaa
aaatagaaatatctccatctggggcaccacgctttatccgaaatatagta
ataaagtagataatccaatcgaacaccaccaccaccaccactaataagga
tcc.

The underlined sequences are, starting from the beginning of the sequence, the NdeI site, the NheI site, the CBG-encoding region, the 6×His tag, and the BamHI site. The CBD resides to be mutated in the next step are in bold-italics.

### Splicing by Overlap Extension (SOE) PCR

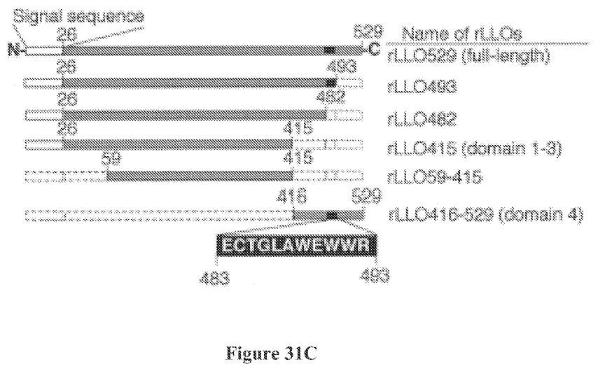

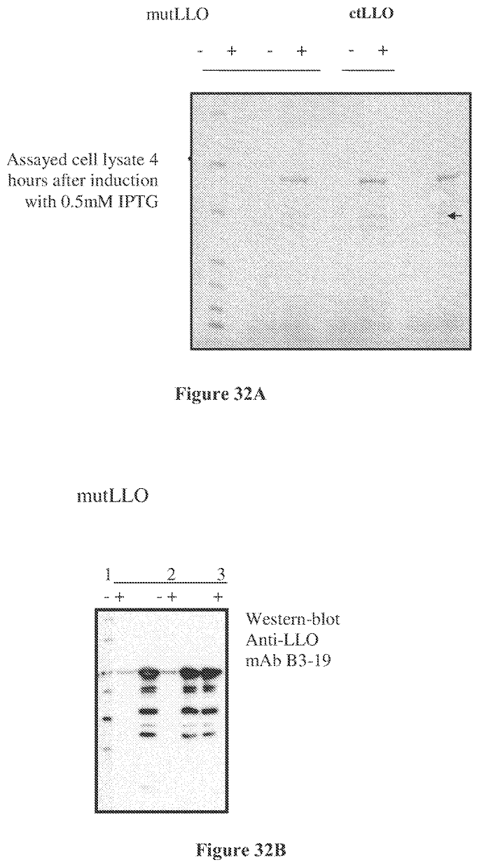

Step 1: PCR reactions #1 and #2 were performed on the pET29b-LLO template. PCR reaction #1, utilizing primers #1 and #2, amplified the fragment between the NheI site and the CBD, inclusive, introducing a mutation into the CBD. PCR reaction #2, utilizing primers #3 and #4, amplified the fragment between the CBD and the BamHI site, inclusive, introducing the same mutation into the CBD (FIG. 31A).

PCR reaction #1 cycle: A) 94° C. 2 min 30 sec, B) 94° C. 30 sec, C) 55° C. 30 sec, D) 72° C. 1 min, Repeat steps B to D 29 times (30 cycles total), E) 72° C. 10 min.

PCR reaction #2 cycle: A) 94° C. 2 min 30 sec, B) 94° C. 30 sec, C) 60° C. 30 sec, D) 72° C. 1 min, Repeat steps B to D 29 times (30 cycles total), E) 72° C. 10 min.

Step 2: The products of PCR reactions #1 and #2 were mixed, allowed to anneal (at the mutated CBD-encoding region), and PCR was performed with primers #1 and #4 for 25 more cycles (FIG. 31B). PCR reaction cycle: A) 94° C. 2 min 30 sec, B) 94° C. 30 sec, C) 72° C. 1 min, Repeat steps B to C 9 times (10 cycles total), Add primers #1 and #4, D) 94° C. 30 sec, E) 55° C. 30 sec, F) 72° C. 1 min, Repeat steps D to F 24 times (25 cycles total), G) 72° C. 10 min

Primer Sequences:
Primer 1:
GCTAGCTCATTTCACATCGT (SEQ ID NO: 64; NheI sequence
is underlined).
Primer 2:
TCT [symbol] TTCCCAAGCTAAACCAGT [symbol] TTCTTTAGCGTAAACATT
AATATT (SEQ ID NO: 65; CBD-encodingsequence is
underlined; mutated codons are in bold-italics).
Primer 3:
GAA [symbol] ACTGGTTTAGCTTGGGAA [symbol] AGAACGGTAATTGATGAC
CGGAAC (SEQ ID NO: 66; CBD-encoding sequence is
underlined; mutated codons are in bold-italics).
Primer 4:
GGATCCTTATTAGTGGTGGTGGTGGTGGTGTTCGATTGG (SEQ
ID NO: 67; BamHI sequence is underlined).

The wild-type CBD sequence is ECTGLAWEWWR (SEQ ID NO: 68).

The mutated CBD sequence is EATGLAWEAAR (SEQ ID NO: 69).

The sequence of the mutated NheI-BamHI fragment is

(SEQ ID NO: 70)
GCTAGCTCATTTCACATCGTCCATCTATTTGCCTGGTAACGCGAGAAATA
TTAATGTTTACGCTAAAGAA [symbol] ACTGGTTTAGCTTGGGAA [symbol] AG
AACGGTAATTGATGACCGGAACTTACCACTTGTGAAAAATAGAAATATC
TCCATCTGGGGCACCACGCTTTATCCGAAATATAGTAATAAAGTAGATA
ATCCAATCGAACACCACCACCACCACCACTAATAAGGATCC.

### Example 26: Replacement of Part of the LLO CBD with a CTL Epitope

Site-directed mutagenesis was performed on LLO to replace 9 amino acids (AA) of the CBD with a CTL epitope from the antigen NY-ESO-1. The sequence of the CBD (SEQ ID NO: 68) was replaced with the sequence

(SEQ ID NO: 71
ESLLMWITQCR;

mutated residues underlined), which contains the HLA-A2 restricted epitope 157-165 from NY-ESO-1, termed “ctLLO.”

The subcloning strategy used was similar to the previous Example.

The primers used were as follows:

Primer 1:
GCTAGCTCATTTCACATCGT (SEQ ID NO: 64; NheI sequence
is underlined).
Primer 2:
TCT [symbol] TTCTTTAGCGTAAACATTA
ATATT (SEQ ID NO: 72; CBD-encoding sequence is
underlined; mutated (NY-ESO-1) codons are in
bold-italics).
Primer 3:
GAA [symbol] AGAACGGTAATTGATGACC
GGAAC (SEQ ID NO: 73; CBD-encoding sequence is
underlined; mutated (NY-ESO-1) codons are in
bold-italics).
Primer 4:
GGATCCTTATTAGTGGTGGTGGTGGTGGTGTTCGATTGG (SEQ
ID NO: 67; BamHI sequence is underlined).

The sequence of the resulting NheI/BamHI fragment is as follows:

(SEQ ID NO: 74)
GCTAGCTCATTTCACATCGTCCATCTATTTGCCTGGTAACGCGAGAAATA
TTAATGTTTACGCTAAAGAA [symbol] AG
AACGGTAATTGATGACCGGAACTTACCACTTGTGAAAAATAGAAATATCT
CCATCTGGGGCACCACGCTTTATCCGAAATATAGTAATAAAGTAGATAAT
CCAATCGAACACCACCACCACCACCACTAATAAGGATCC.

### Example 27: mutLLO and ctLLO are able to be Expressed and Purified in E. coli Expression Systems

To show that mutLLO and ctLLO could be expressed in E. coli, E. coli were transformed with pET29b and induced with 0.5 mM IPTG, then cell lysates were harvested 4 hours later and the total proteins were separated in a SDS-PAGE gel and subject to Coomassie staining (FIG. 32A) and anti-LLO Western blot, using monoclonal antibody B3-19 (FIG. 32B). Thus, LLO proteins containing point mutations or substitutions in the CBD can be expressed and purified in E. coli expression systems.

### Example 28: mutLLO and ctLLO Exhibit Significant Reduction in Hemolytic Activity

### Materials and Experimental Methods

### Hemolysis Assay

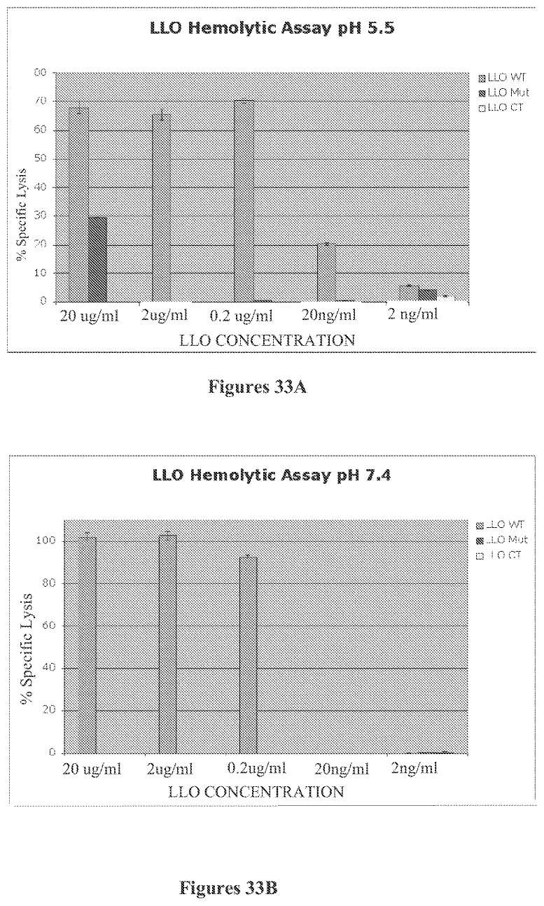

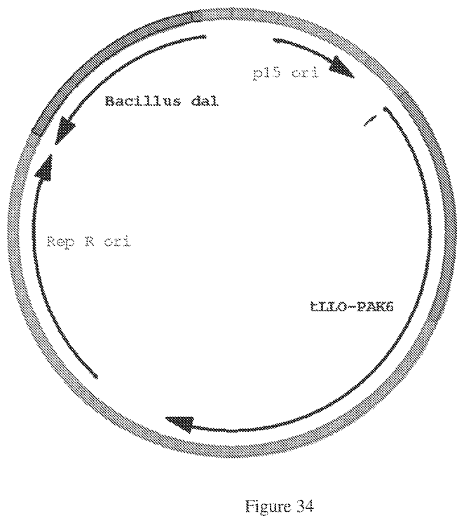

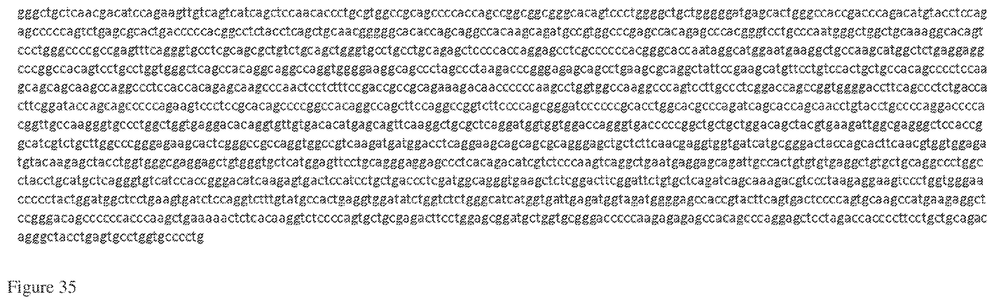

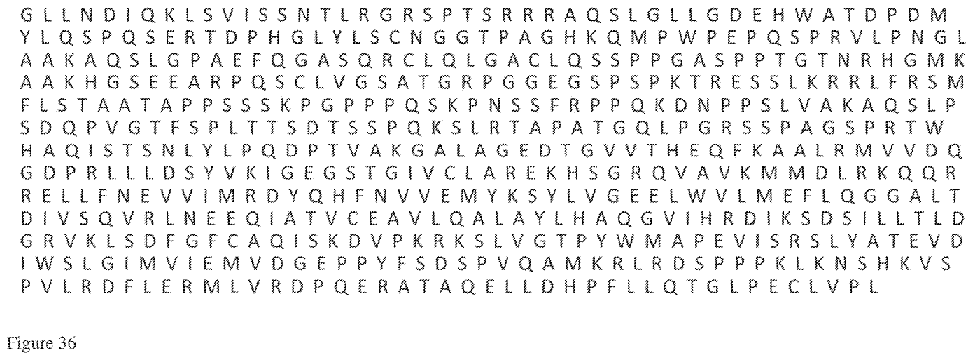

1. Wild-type and mutated LLO were diluted to the dilutions indicated in FIGS. 33A-B in 900 μl of 1×PBS-cysteine (PBS adjusted to pH 5.5 with 0.5 M Cysteine hydrochloride or was adjusted to 7.4). 2. LLO was activated by incubating at 37° C. for 30 minutes. 3. Sheep red blood cells (200 μl/sample) were washed twice in PBS-cysteine and 3 to 5 times in 1×PBS until the supernatant was relatively clear. 4. The final pellet of sheep red blood cells was resuspended in PBS-cysteine and 100 μl of the cell suspension was added to the 900 μl of the LLO solution (10% final solution). 5. 50 μl of sheep red blood cells was added to 950 μl of water+10% Tween 20 (Positive control for lysis, will contain 50% the amount of lysed cells as the total amount of cells add to the other tubes; “50% control.”) 6. All tubes were mixed gently and incubated at 37° C. for 45 minutes. 7. Red blood cells were centrifuged in a microcentrifuge for 10 minutes at 1500 rpm. 8. A 200 μl aliquot of the supernatant was transferred to 96-well ELISA plate and read at 570 nm to measure the concentration of released hemoglobin after hemolysis, and samples were titered according to the 50% control.

### Results

The hemolytic activity of mutLLO and ctLLO was determined using a sheep red blood cell assay. mutLLO exhibited significantly reduced (between 100-fold and 1000-fold) hemolytic titer at pH 5.5, and undetectable hemolytic activity at pH 7.4. ctLLO exhibited undetectable hemolytic activity at either pH (FIGS. 33A-B).

Thus, point (mutLLO) or substitution (ctLLO) mutation of LLO CBD residues, including C484, W491, and W492, abolishes or severely reduces hemolytic activity. Further, replacement of the CBD with a heterologous antigenic peptide is an effective means of creating an immunogenic carrier of a heterologous epitope, with significantly reduced hemolytic activity relative to wild-type LLO.

### Example 29: Fully Enclosed Single Use Cell Growth System

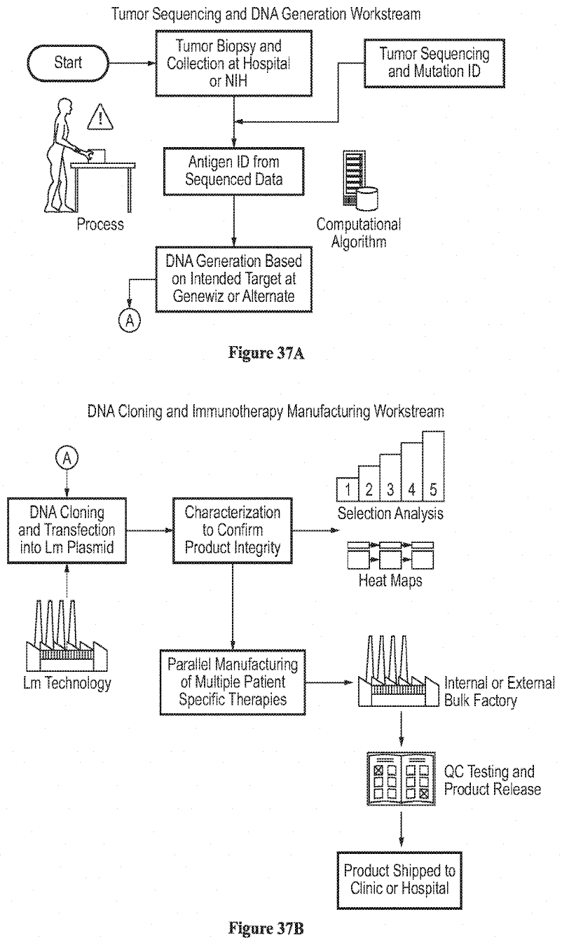

The innovative system leverages readily available bioprocessing components and technologies arranged in a unique configuration to grow the engineered Lm bacteria, concentrate the fermentation broth, wash and purify the cells, exchange the fermentation media for formulation buffer, and dispense the patient-specific doses into ready-to-use IV bags using a single fully enclosed system. This type of system provides a complete segregation and control of each patient's immunotherapy. This system is particularly well suited for integration in the overall work stream of identification and clinical use of personalized neo-epitope targeting immunotherapeutics (FIG. 37 A-B).

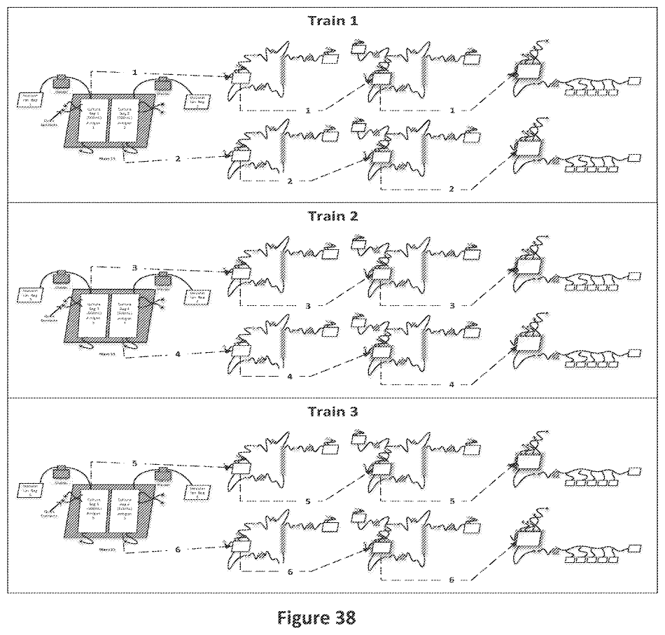

The custom designed system is assembled using single use bioprocessing bags, patient IV bags, sampling bags, tubing, filters, quick connectors, and sensors. Its small footprint allows manufacture for an individual patient but can be replicated to manufacture product for multiple patients in parallel (FIG. 38). The entire assembly is comprised of 4 sections: 1) Inoculation and Fermentation, 2) Concentration, 3) Diafiltration, and 4) Drug Product Fill. Since the system has a fully enclosed fluid flow path and is sterilized prior to use, final formulated immunotherapies are dispensed directly into IV bags, frozen and shipped to the healthcare center. Therefore, this eliminates the need for the typical fill/finish and packaging involved when dispensing into vials or pre-filled syringes. This addresses the expectation for rapid turnaround and delivery to the patient.

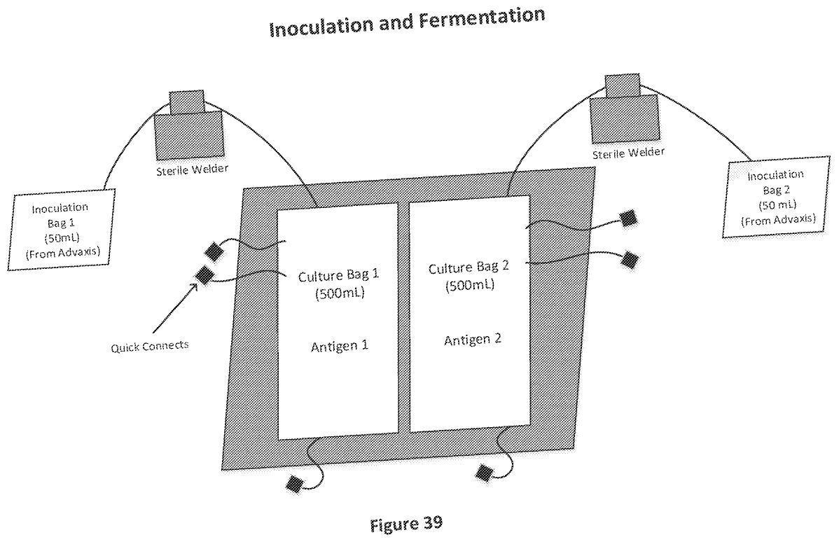

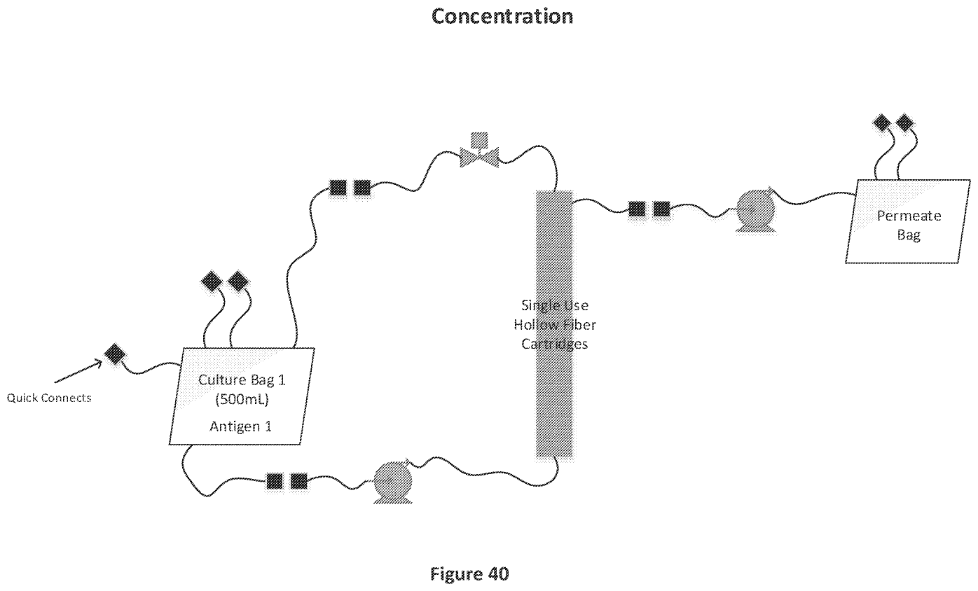

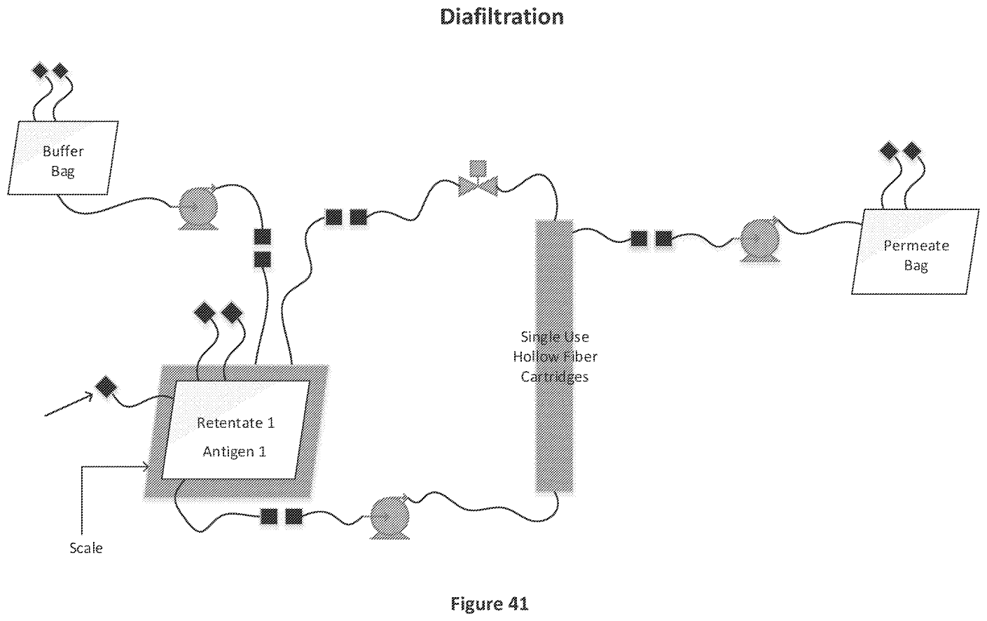

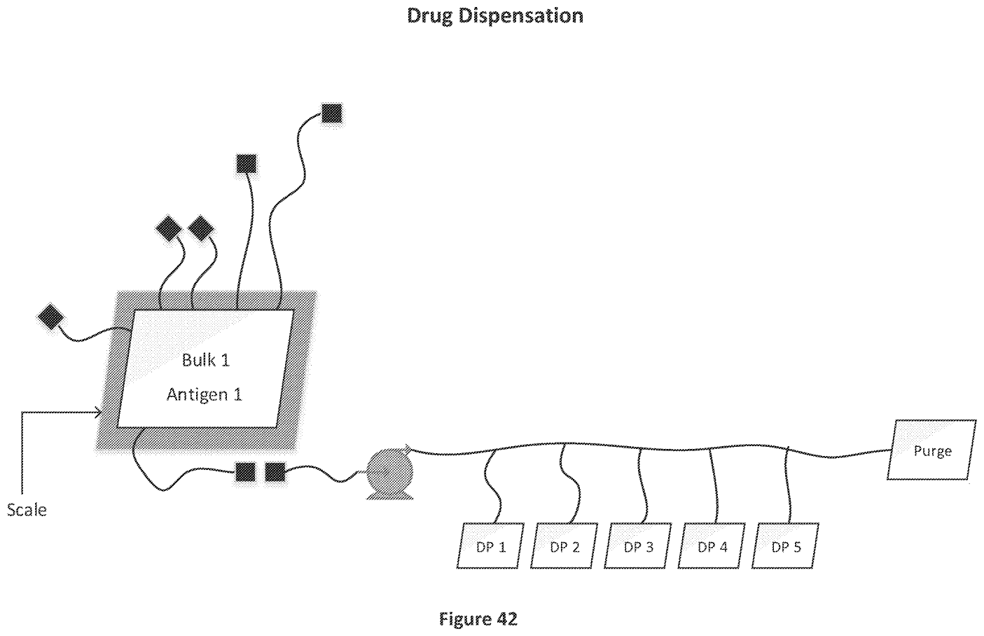

The Inoculation and Fermentation section of the assembly (FIG. 39) is filled with growth media and warmed to the specified temperature. The cell bank is then inoculated into either a single use rocking style bag fermentor or into a single use agitated bioreactor vessel. Once the bacteria grows to a specific density, the Concentration section of the assembly (FIG. 40) is used to remove the fermentation media and concentrate the batch using a hollow fiber filter. A wash/formulation buffer bag is connected to the Diafiltration section of the assembly (FIG. 41) and the bacterial cells are washed/purified, the remaining media is exchanged with formulation buffer via a cross flow filtration in the hollow fiber filter, and the product is diluted to the final concentration Finally, the batch is aliquoted into sterile single use IV bags and sampling bags for QC testing using the Drug Product Fill section of the assembly (FIG. 42). The patient-specific immunotherapy is supplied frozen in a small volume parenteral IV bag containing a pure culture strain of the live attenuated engineered Lm bacteria at a specified concentration. Prior to patient administration, the IV bag is thawed, cells re-suspended, and the required dose withdrawn with a syringe and added to the larger infusion IV bag.

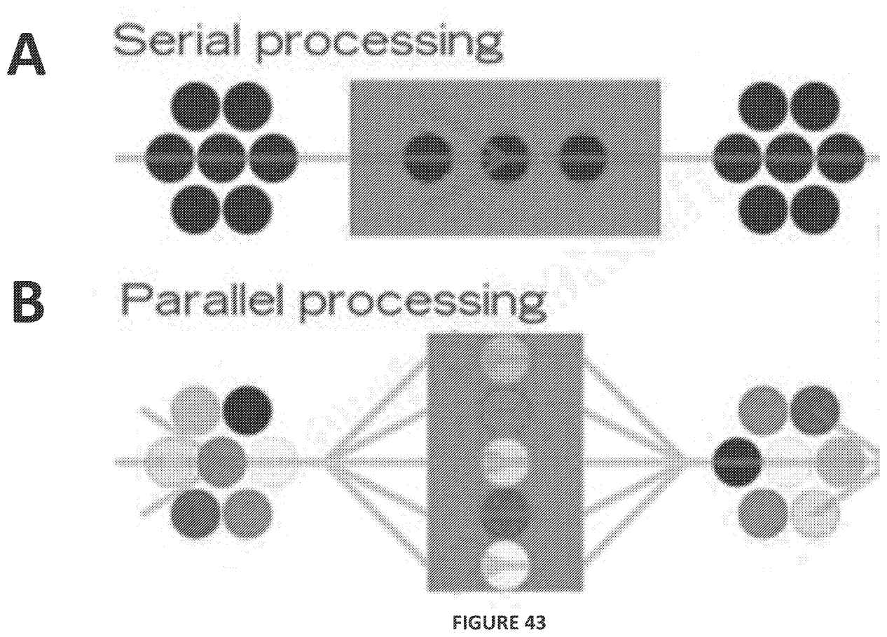

Several fully enclosed assemblies are used in parallel to manufacture personalized immunotherapeutic compositions either for several patients or for a single patient (FIG. 43). In order to increase throughput, additional rockers or agitated vessel bioreactors systems are added to the processing train, as required (see e.g. FIG. 38).

The fully enclosed design of the growth system allow complete quality control of immunotherapeutic compositions while in the process of manufacture, resulting in additional time savings. A full analytical control strategy is implemented in parallel with growing Listeria delivery vector (Table 6). Thus the dispensed product is ready for immediate delivery to the patient with no additional testing required.

TABLE 6
Analytical Control Strategy

| Parameter | Quality Attribute | Test Method | Test Duration | | Comment |
|---|---|---|---|---|---|
| Identity | Plasmid ID | PCR | 5 | days | 3 days + 2VCC |
| Safety | Attenuation | Macrophage or THP1 | 5 | days | 3 days + 2VCC |
| General | Solution Appearance | | 1 | day | |
| General | pH | | 1 | day | |
| General | Osmolality | | 1 | day | |
| Content | Fill Weight | In Process Test | 0 | day | |
| Content | Viable Cell Count | Plate | 2 | days | |
| Content | Plasmid Copy Number | PCR | 5 | days | 3 days + 2VCC |
| Potency | In vitro Potency | J774 Infectivity | 5-10 | days | 3-7 days + 2VCC |
| | | Intracellular Express | | | |
| Purity | Plasmid Stability | | 5 | days | |
| Purity | Microbial Purity | Plate Method | 21 | days | Need Rapid ID |
| | | | | | method |
| Purity | Percent of Live and | | 5 | days | |
| | Dead Cells | | | | |
| Safety | Endotoxin | | 5 | days | |

### Example 30: Construction of a Neo-Epitope Expression Vector

Constructing the Lm vector comprising one or more neo-epitopes is performed using the steps detailed below.

### Whole Genome Sequencing

First, comparative whole genome sequencing including locating non-synonymous mutations present in approximately >20% of tumor cells is performed and the results are provided in FASTA format. Matched normal/tumor samples from whole exomes are sequenced by an outside vendor, and output data is given in the preferred FASTA format listing all neo-antigens as 21 amino acid sequence peptides, for example a peptide having 10 non-mutant amino acids on either side of a mutant amino acid. Also included are patient HLA types.

DNA and RNA from a biological sample obtained from human tissue (or any non-human animal) are extracted in triplicates. Another source of neo-antigens could be from sequencing metastases or circulating tumor cells. They may contain additional mutations that are not resident in the initial biopsy but could be included in the vector to specifically target cytotoxic T cells (CTC's) or metastases that have mutated differently than the primary biopsy that was sequenced. Triplicates of each sample are sequenced by DNA exome sequencing. In brief, 3 μg purified genomic DNA (gDNA) are fragmented to about 150-200 bp using an ultrasound device. Fragments are end repaired, 5′ phosphorylated, 3′ adenylated, and then Illumina paired end adapters are ligated to the gDNA fragments according to the manufacturer's instructions. Enriched pre capture and flow cell specific sequences are added using Illumina PE PCR primers. About 500 ng of adapter ligated, PCR enriched gDNA fragments are hybridized to biotinylated exome (human exome or any other non-human animal exome e.g. mouse, guinea pig, rat, dog, sheep). RNA library baits for 24 hrs at 65° C. Hybridized gDNA/RNA bait complexes are then removed using streptavidin coated magnetic beads, washed and the RNA baits cleaved off. These eluted gDNA fragments are PCR amplified and then sequenced on an Illumina sequencing apparatus.

### RNA Gene Expression Profiling (RNA-Seq)

Barcoded mRNA-seq cDNA libraries are prepared in triplicates from a total of about 5 μg of total RNA, then, in brief, mRNA are isolated and fragmented. Following, mRNA fragments are converted to cDNA and connected to specific Illumina adaptors, clustered and sequenced according to standard illumine protocol. The output sequence reads are aligned to a referenced sequence (RefSeq). Genome alignments and transcriptome alignments are made. Reads are also aligned to exon-exon junctions. Expression values are determined by intersecting read coordinates with those of RefSeq transcripts, counting overlapping exon and exon junction reads, and normalized to standard normalizing units such as RPKM expression units (Reads which map per Kilobase of transcript per Million mapped reads).

### Detecting Mutations

Fragments of isolated gDNA from a disease or condition bearing tissue sample are aligned to referenced matched gDNA of a healthy tissue, by vendor available software, e.g. Samtools, GATK, and Somatic Sniper.

About 10 flanking amino acids on each side of the detected mutation are incorporated to accommodate class 1 MHC-1 presentation, in order to provide at least some of the different HLA TCR reading frames.

Table 7 shows a sample list of 50 neo-epitope peptides wherein each mutation is indicated by a Bolded amino acid letter and is flanked by 10 amino acids on each side providing a 21 amino acid peptide neo-epitope.

TABLE 7

| Name | Sequence1 | Sequence ID NO: |
|---|---|---|
| MUT1 | FMVAVAHVAAFLLEDRAVCV | 88 |
| MUT2 | AENVEQVLVTSIQGAVDYPDP | 89 |
| MUT3 | SFKKKFEECQHNIIKLQNGHT | 90 |
| MUT4 | SALIESLNQKTQSTGDHPQPT | 91 |
| MUT5 | KAYLPVNESFAFTADLRSNTG | 92 |
| MUT6 | HTLLEITEESGAVLVDKSDSD | 93 |
| MUT7 | SVMCTYSPPLDKLFCQLAKTC | 94 |
| MUT8 | ESGKHKYRQTAMFTATMPPAV | 95 |
| MUT9 | AAPSAASSPADVQSLKKAMSS | 96 |
| MUT10 | SQLFSLNPRGRSLVTAGRIDR | 97 |
| MUT11 | SLARGPLSEAGLALFDPYSKE | 98 |
| MUT12 | QKKLCHLSSTGLPRETIASLP | 99 |
| MUT13 | LTASNMEGKSWPSEVLVCTTS | 100 |
| MUT14 | YAAQQHETFLTNGDRAGFLIG | 101 |
| MUT15 | QAKVPFSEETQNLILPYISDM | 102 |
| MUT16 | CNRAGEKHCFSSNEAARDFGG | 103 |
| MUT17 | RNPQFLDPVLAYLMKGLCEKP | 104 |
| MUT18 | LECERGKQEAKLLAERSRFED | 105 |
| MUT19 | APLEWLRYFDKKELELMLCGM | 106 |
| MUT20 | KAFLHWYTGEAMDEMEFTEAE | 107 |
| MUT21 | DEVALVEGVQSLGFTYLRLKD | 108 |
| MUT22 | DFSQLQRNILPSNPRVTRFHI | 109 |
| MUT23 | ISTNGSFIRLLDAFKGVVMHT | 110 |
| MUT24 | ITPPTTTTKKARVSTPKPATP | 111 |
| MUT25 | NYNTSHLNNDVWQIFENPVDW | 112 |
| MUT26 | QKTLHNLLRKVVPSFSAEIER | 113 |
| MUT27 | VELCPGNKYEMRRHGTTHSLV | 114 |
| MUT28 | GIDKLTQLKKPFLVNNKINKI | 115 |
| MUT29 | GTTILNCFHDVLSGKLSGGS | 116 |
| MUT30 | PSFQEFVDWENVSPELNSTDQ | 117 |
| MUT31 | PALVEEYLERGNFVANDLDWL | 118 |
| MUT32 | ELKACKPNGKRNPYCEVSMGS | 119 |
| MUT33 | SPFPAAVILRDALHMARGLKY | 120 |
| MUT34 | QQLDTYILKNVVAFSRTDKYR | 121 |
| MUT35 | SFVGQTRVLMINGEEVEETEL | 122 |
| MUT36 | AFFINFIAIYHHASRAIPFGT | 123 |
| MUT37 | GLALPNNYCDVCLGDSKINKK | 124 |
| MUT38 | EGQISIAKYENCPKDNPMYYC | 125 |
| MUT39 | NFKRKRVAAFQKNLIEMSELE | 126 |
| MUT40 | KMKGELGMMLILQNVIQKTTT | 127 |
| MUT41 | SIECKGIDKEINESKNTHLDI | 128 |
| MUT42 | ELEAAIETVVCTFFTFAGREG | 129 |
| MUT43 | SLSHREREQMKATLNYEDHCF | 130 |
| MUT44 | HIKAFDRTFANNPGPMVVFAT | 131 |
| MUT45 | ITSNFVIPSEYWVEEKEEKQK | 132 |
| MUT46 | GLVTFQAFIDVMSRETTDTDT | 133 |
| MUT47 | HLLGRLAAIVGKQVLLGRKVV | 134 |
| MUT48 | HWNDLAVIPAGVVHNWDFEPR | 135 |
| MUT49 | SMDHKTGTIAMQNTTQLRSRY | 136 |
| MUT50 | QPLRRLVLHVVSAAQAERLAR | 137 |

1 Bolded letter indicates mutated amino acid

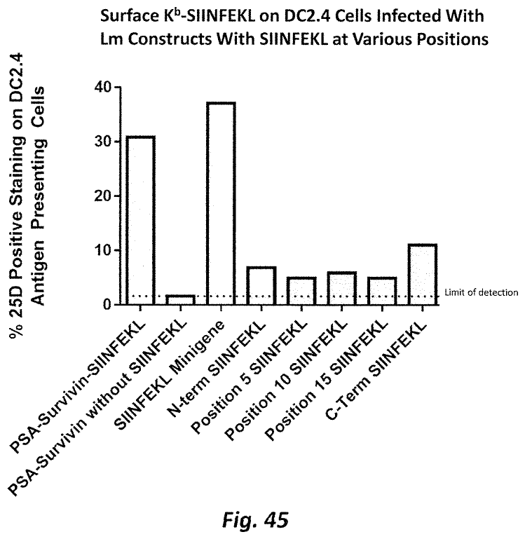

Output FASTA file is used to design patient-specific constructs, either manually or by programmed script according to one or more of criteria detailed below. The programmed script automates the creation of the personalized plasma construct containing one or more neo-epitopes for each subject using a series of protocols (FIG. 45). The output FASTA file is inputted and after running the protocols, the DNA sequence of a LM vector including one or more neo-epitopes is outputted. The software program is useful for creating personalized immunotherapy for each subject.

Prioritization of Neo-Epitopes for Incorporation into Constructs.

Neo-epitopes are scored by Kyte and Doolittle hydropathy index 21 amino acid window, all scoring above cutoff (around 1.6) are excluded as they are unlikely to be secretable by Listeria monocytogenes. The remaining 21 amino acid long peptides are then scored for their ability to bind patient HLA (for example by using IEDB, Immune epitope database and analysis source, www.iedb.org/) and ranked by best MHC binding score from each 21 amino acid sequence peptide. Cut-offs may be different for different expression vectors such as Salmonella.

Determination of the number of constructs vs. mutational burden, are performed to determine efficiency of expression and secretion of neo-epitopes. Ranges of linear neo-epitopes are tested, starting with about 50 epitopes per vector. In certain cases constructs will include at least one neo-epitope per vector. The number of vectors to be used is determined considering for example the efficiency of translation and secretion of multiple epitopes from a single vector, and the MOI needed for each Lm vector harboring specific neo-epitopes, or in reference to the number of neo-epitopes. Another consideration can be by predefining groups of known tumor-associated mutations/mutations found in circulating tumor cells/known cancer “driver” mutations/known chemotherapy resistance mutations and giving them priority in the 21 amino acid sequence peptide selection. This can be accomplished by screening identified mutated genes against the COSMIC (Catalogue of somatic mutations in cancer, cancer.Sanger.ac.uk) or Cancer Genome Analysis or other similar cancer-associated gene database. Further, screening for immunosuppressive epitopes (T-reg epitopes, IL-10 inducing T helper epitopes, etc.) is utilized to de-selected or to avoid immunosuppressive influences on the vector. Selected codons are codon optimized to efficient translation and secretion according to specific Listeria strain. Example for codons optimized for L. monocytogenes as known in the art is presented in table 8.

TABLE 8
Preliminary Listeria monocytogenes
preferred (most common) codon table

A = GCA
C = TGT
D = GAT
E = GAA
F = TTC
G = GGT
H = CAT
I = ATT
K = AAA
L = TTA
M = ATG
N = AAC
P = CCA
Q = CAA
R = CGT
S = TCT
T = ACA
V = GTT
W = TGG
Y = TAT
STOP = TAA

The remaining 21 amino acid peptide neo-epitopes are assembled into a pAdv134-MCS (SEQ ID NO: 138) plasmid, or optionally into pAdv134, exchanging the LLO-E7 cassette as shown in Example 8 above, to create the tLLO-neo-epitope-tag fusion polypeptide. The compatible insert as an amino acid sequence and the whole insert are rechecked by Kyte and Doolittle test to confirm no hydropathy problems across the whole construct. If needed, the insert order is rearranged or the problem 21 amino acid sequence peptides is removed from construct.

The construct amino acid sequence is reverse translated into the corresponding DNA sequence for DNA synthesis/cloning into pAdv134-MCS SEQ ID NO: 138:

cggagtgtatactggcttactatgaggcactgatgagggtgtcagtgaag
tgcttcatgtggcaggagaaaaaaggctgcaccggtgcgtcagcagaata
tgtgatacaggatatattccgcacctcgctcactgactcgctacgctcgg
tcgttcgactgcggcgagcggaaatggcttacgaacggggcggagatacc
tggaagatgccaggaagatacttaacagggaagtgagagggccgcggcaa
agccgtattccataggctccgcccccctgacaagcatcacgaaatctgac
gctcaaatcagtggtggcgaaacccgacaggactataaagataccaggcg
taccccctggcggctccctcgtgcgctctcctgacctgccatcggatacc
ggtgtcattccgctgttatggccgcgtagtctcattccacgcctgacact
cagaccgggtaggcagttcgctccaagctggactgtatgcacgaaccccc
cgttcagtccgaccgctgcgccttatccggtaactatcgtcttgagtcca
acccggaaagacatgcaaaagcaccactggcagcagccactggtaattga
tttagaggagttagtcttgaagtcatgcgccggttaaggctaaactgaaa
ggacaagattggtgactgcgctcctccaagccagttacctcggacaaaga
gaggtagctcagagaaccacgaaaaaccgccctgcaaggcggattacgat
tcagagcaagagattacgcgcagaccaaaacgatctcaagaagatcatct
tattaatcagataaaatatactagccctccatgattagtatattcctatc
ttaaagttactatatgtggaggcattaacatagttaatgacgtcaaaagg
atagcaagactagaataaagctataaagcaagcatataatattgcgatca
tcatagaagcgaatttcgccaatattataattatcaaaagagaggggtgg
caaacggtataggcattattaggttaaaaaatgtagaaggagagtgaaac
ccatgaaaaaaataatgctagtattattacacttatattagttagtctac
caattgcgcaacaaactgaagcaaaggatgcatctgcattcaataaagaa
aattcaatttcatccatggcaccaccagcatctccgcctgcaagtcctaa
gacgccaatcgaaaagaaacacgcggatgaaatcgataagtatatacaag
gattggattacaataaaaacaatgtattagtataccacggagatgcagtg
acaaatgtgccgccaagaaaaggttacaaagatggaaatgaatatattga
gtggagaaaaagaagaaatccatcaatcaaaataatgcagacattcaaga
gtgaatgcaatttcgagcctaacctatccaggtgctctcgtaaaagcgaa
ttcggaattagtagaaaatcaaccagatgactccctgtaaaacgtgattc
attaacactcagcattgatttgccaggtatgactaatcaagacaataaaa
tagagtaaaaaatgccactaaatcaaacgttaacaacgcagtaaatacat
tagtggaaagatggaatgaaaaatatgctcaagcttatccaaatgtaagt
gcaaaaattgattatgatgacgaaatggcttacagtgaatcacaattaat
tgcgaaataggtacagcatttaaagctgtaaataatagcttgaatgtaaa
cttcggcgcaatcagtgaagggaaaatgcaagaagaagtcattagattaa
acaaatttactataacgtgaatgttaatgaacctacaagaccaccagatt
atcggcaaagctgttactaaagagcagagcaagcgcaggagtgaatgcag
aaaatcctcctgcatatatctcaagtgtggcgtatggccgtcaagatatt
tgaaattatcaactaattcccatagtactaaagtaaaagctgcattgatg
ctgccgtaagcggaaaatctgtctcaggtgatgtagaactaacaaatatc
atcaaaaattcaccacaaagccgtaatttacggaggaccgcaaaagatga
agttcaaatcatcgacggcaacctcggagacttacgcgatattagaaaaa
aggcgctactataatcgagaaacaccaggagacccattgcttatacaaca
aacacctaaaagacaatgaattagctgttattaaaaacaactcagaatat
attgaaacaacttcaaaagcttatacagatggaaaaattaacatcgatca
ctctggaggatacgttgctcaattcaacatttcagggatgaagtaaatta
tgatCTCGAGGAGCTCCTGCAGTCTAGAGTCGACACTAGTGGATCCAGAT
CTCCCGGGccactaactcaacgctagtagtggatttaatcccaaatgagc
caacagaaccagaaccagaaacagaacaagtaacattggagttagaaatg
gaagaagaaaaaagaatgatttcgtgtgaataatgcacgaaatcattgct
tattatttaaaaagcgatatactagatataacgaaacaacgaactgaata
aagaatacaaaaaaagagccacgaccagttaaagcctgagaaactttaac
tgcgagccttaattgattaccaccaatcaattaaagaagtcgagacccaa
aataggtaaagtatttaattactttattaatcagatacttaaatatctgt
aaacccattatatcgggtattgaggggatttcaagtcataagaagatacc
aggcaatcaattaagaaaaacttagttgattgccattagagtgattcaac
tagatcgtagcttctaactaattaattacgtaagaaaggagaacagctga
atgaatatcccttttgagtagaaactgtgcttcatgacggcagttaaagt
acaaatttaaaaatagtaaaattcgctcaatcactaccaagccaggtaaa
agtaaaggggctatattgcgtatcgctcaaaaaaaagcatgattggcgga
cgtggcgttgactgacttccgaagaagcgattcacgaaaatcaagataca
tttacgcattggacaccaaacgatatcgttatggtacgtatgcagacgaa
aaccgttcatacactaaaggacattctgaaaacaatttaagacaaatcaa
taccactttattgattagatattcacacggaaaaagaaactatttcagca
agcgatatataacaacagctattgatttaggattatgcctacgttaatta
tcaaatctgataaaggttatcaagcatattagattagaaacgccagtcta
tgtgacttcaaaatcagaatttaaatctgtcaaagcagccaaaataatct
cgcaaaatatccgagaatattaggaaagtcatgccagttgatctaacgtg
caatcattagggattgctcgtataccaagaacggacaatgtagaattatt
gatcccaattaccgttattcatcaaagaatggcaagattggtcatcaaac
aaacagataataagggcatactcgttcaagtctaacggattaagcggtac
agaaggcaaaaaacaagtagatgaaccctggataatctcttattgcacga
aacgaaattacaggagaaaagggatagtagggcgcaatagcgttatgata
ccctctcatagcctactttagttcaggctattcaatcgaaacgtgcgaat
ataatatgatgagataataatcgattagatcaacccttagaagaaaaaga
agtaatcaaaattgttagaagtgcctattcagaaaactatcaaggggcta
atagggaatacattaccattcatgcaaagcagggtatcaagtgatttaac
cagtaaagatttatagtccgtcaagggtggataaattcaagaaaaaaaga
agcgaacgtcaacgtgacatagtcagaatggaaagaagatttaatggctt
atattagcgaaaaaagcgatgtatacaagccttatttagcgacgaccaaa
aaagagattagagaagtgctaggcattcctgaacggacattagataaatt
gctgaaggtactgaaggcgaatcaggaaattactttaagattaaaccagg
aagaaatggtggcattcaacttgctagtgttaaatcattgagctatcgat
cattaaattaaaaaaagaagaacgagaaagctatataaaggcgctgacag
cttcgataatttagaacgtacatttattcaagaaactctaaacaaattgg
cagaacgccccaaaacggacccacaactcgatttgatagctacgatacag
gctgaaaataaaacccgcactatgccattacatttatatctatgatacgt
gatgatactagctggctagcttaattgcttatatttacctgcaataaagg
atacttacttccattatactcccattaccaaaaacatacggggaacacgg
gaacttattgtacaggccacctcatagttaatggtacgagccacctgcaa
tctcatccatggaaatatattcatccccctgccggcctattaatgtgact
tagtgcccggcggatattcctgatccagctccaccataaattggtccatg
caaattcggccggcaattacaggcgattcccacacaaggatgtcggtccc
atcaattacggagccagccgtccgcatagcctacaggcaccgtcccgatc
catgtgtcataccgctgtgtactcggctccgtagctgacgctctcgccat
tctgatcagatgacatgtgacagtgtcgaatgcagggtaaatgccggacg
cagctgaaacggtatctcgtccgacatgtcagcagacgggcgaaggccat
acatgccgatgccgaatctgactgcattaaaaaagccattacagccggag
tccagcggcgctgacgcgcagtggaccattagattcataacggcagcgga
gcaatcagctcataaagcgctcaaactgcattaagaaatagcctctacta
ttcatccgctgtcgcaaaatgggtaaataccccatgcactttaaacgagg
gagcggtcaagaattgccatcacgactgaacttcacctctgatttacacc
aagtctgacatccccgtatcgaccacagatgaaaatgaagagaaccatta
cgtgtggcgggctgcctcctgaagccattcaacagaataacctgttaagg
tcacgtcatactcagcagcgattgccacatactccgggggaaccgcgcca
agcaccaatataggcgccacaatcccatttgcgcagtgaaatcgcttcat
ccaaaatggccacggccaagcatgaagcacctgcgtcaagagcagccatg
ctgatctgcatcaccatgcccgtaggcgtttgctttcacaactgccatca
agtggacatgttcaccgatatgttttttcatattgctgacattttccttt
atcacggacaagtcaatttccgcccacgtatctctgtaaaaaggttttgt
gctc atggaaaactcctctcttttttcagaaaatcccagtacgtaatta
agtatttgagaattaattttatattgattaatactaagtttacccagttt
tcacctaaaaaacaaatgatgagataatagctccaaaggctaaagaggac
tataccaactatttgttaattaa; Capital letters refer to
multi-cloning site by outside vendor.

Individual 21 amino acid peptides sequences and the SIINFEKL-6×His tag DNA sequences (for example SEQ ID NO: 87) are optimized for expression and secretion in L. monocytogenes while the 4× glycine linker sequences are one of eleven preset DNA sequences (G1-G11, SEQ ID NO: 76-86). Linker sequence codons are varied to avoid excess repetition to better enable DNA synthesis. Examples of the different sequence codons (G1-G11, SEQ ID NO: 76-86) for 4× glycine linkers are presented in Table 9.

TABLE 9
4x glycine linker DNA sequences and terminal
tag sequence

| Name | Sequence | Sequence ID NO: |
|---|---|---|
| G1 | GGTGGTGGAGGA | 76 |
| G2 | GGTGGAGGTGGA | 77 |
| G3 | GGTGGAGGAGGT | 78 |
| G4 | GGAGGTGGTGGA | 79 |
| G5 | GGAGGAGGTGGT | 80 |
| G6 | GGAGGTGGAGGT | 81 |
| G7 | GGAGGAGGAGGT | 82 |
| G8 | GGAGGAGGTGGA | 83 |
| G9 | GGAGGTGGAGGA | 84 |
| G10 | GGTGGAGGAGGA | 85 |
| G11 | GGAGGAGGAGGA | 86 |
| C-terminal | ARSIINFEKLSHHHHHH | 87 |
| SIINFEKL | | |
| and 6xHis | | |
| AA sequence | | |

Each neo-epitope is connected with a linker sequence to the following neo-epitope encoded on the same vector. The final neo-epitope in an insert is fused to a TAG sequence followed by a stop codon. The TAG fused is set forth in SEQ ID NO: 87, a C-terminal SIINFEKL and 6×His amino acid sequence. The TAG allows for easy detection of the tLLO-neo-epitope during for example secretion from the Lm vector or when testing construct for affinity to specific T-cells, or presentation by antigen presenting cells. The linker is 4× glycine DNA sequence, selected from a group comprising G1-G11 (SEQ ID NO: 76-86) accordingly, or any combination thereof.

If there are more usable 21 amino acid peptides than can fit into a single plasmid (maximum payload currently being tested), the different 21 amino acid peptides are designated into 1^{st}, 2^{nd}, etc. construct by priority rank as needed/desired. The priority of assignment to one of multiple vectors composing the entire set of desired neo-epitopes is determined based on factors like relative size, priority of transcription, and overall hydrophobicity of the translated polypeptide.

In one embodiment, the construct structure disclosed herein comprises a nucleic acid sequence encoding a N terminal truncated LLO fused to one or more 21 mer neo-epitope(s) amino acid sequence flanked by a linker sequence and followed by at least one second neo epitope flanked by another linker and terminated by a SIINFEKL-6×His tag- and 2 stop codons closing the open reading frame: pHly-tLLO-21mer #1-4× glycine linker G1-21mer #2-4× glycine linker G2- . . . -SIINFEKL-6×His tag-2× stop codon. In another embodiment, the above construct's expression is driven by an hly gene promoter sequence or other suitable promoter sequence known in the art and further disclosed herein. It will be appreciated by a skilled artisan that each 21 mer neo-epitope sequence may also be fused to an immunogenic polypeptide such as a tLLO, truncated ActA or PEST amino acid sequence disclosed herein.

Different linker sequences are distributed between the neo-epitopes for minimizing repeats. This reduces possible secondary structures thereby allowing efficient transcription, translation, secretion, maintenance, or stabilization of the plasmid including the insert within the Lm recombinant vector strain population.

DNA synthesis is achieved by ordering nucleotide sequence from a vendor comprising the construct including the open reading frame comprising tLLO or tActA or ActA or PEST amino acid sequence fused to at least one neo-epitope. Additionally or alternatively multiple neo-epitopes are separated by one or more linker 4× glycine sequences. Additionally or alternatively inserts are constructed to comprise the desired sequence by molecular biology technics for example: by sewing PCR with specific over lapping primers and specific primers, or ligating different nucleotide sequences by an appropriate enzyme (e.g., Ligase), optionally following dissection by restriction enzymes, and any combination thereof.

In an embodiment different linker sequences are distributed between the neo-epitopes for minimizing repeats. This reduces possible secondary structures thereby allowing efficient transcription, translation, secretion, maintenance, or stabilization of the plasmid including the insert within the Lm recombinant vector strain population.

Selected DNA inserts are synthesized by techniques standard in the art (e.g., PCR, DNA replication—bio-replication, oligonucleotide chemical synthesis) and cloned to a plasmid, for example as presented in Example 8. The plasmid is then transfected or conjugated into Lm vector. Additionally or alternatively, the insert is integrated into a phage vector and inserted into Lm vector by phage infection. Confirmation of the construct is performed utilizing techniques known in the art, for example bacterial colony PCR with insert specific primers, or purifying said plasmid and sequencing at least a portion comprising the insert.

### Example 31: Expression of Neo-Epitopes from Neo-Epitope Expression Vector

As cancer is driven by mutations, the capability of providing a comprehensive map of somatic mutations in individual tumors provides a powerful tool to better understand and intervene against cancer. Human cancers carry 10s to 100s of non-synonymous mutations. See, e.g., Castle et al. (2012) Cancer Res. 72(5):1081-1091, herein incorporated by reference in its entirety for all purposes. However, shared mutations among patients are rare, and the great majority of mutations are patient-specific, which has hindered exploitation of the mutanome for the development of broadly applicable drugs.

In this example, neo-epitope expression vectors were constructed as in Example 30 based on approximately 200 non-synonymous mutations identified in non-small-cell lung cancer tissue that are not present in healthy lung tissue. Tissues came from UMassMed cancer center of excellence tissue bank (http://www.umassmed.edu/ccoe/core-services/tissue-and-tumor-band/banked-tumor-by-organ-of-origin). Others typically screen based on predictive algorithms for immunogenicity of the epitopes. These algorithms are at best 20% accurate in predicting which peptide will generate a T cell response. This is done because they cannot include all 200 mutations. Here, all mutations could be included, so no screening/predictive algorithms were used. Screening was performed for hydrophobicity, to determine what is likely to be secretable by the Lm strain (i.e. not too hydrophobic). The non-synonymous mutations (neo-epitopes) are provided in the table below.

| | | | SEQ ID NO | SEQ ID NO for |
|---|---|---|---|---|
| | | Hydropathy | for Amino | Nucleotide |
| | Name | Score | Acid Sequence | Sequence |
| 1 | >RERE\|p.R523P\|nonsynonymous SNV [mutant] | −0.852 | 140 | 141 |
| 2 | >CA6\|p.M68V\|nonsynonymous SNV [mutant] | −0.814 | 142 | 143 |
| 3 | >AKR7A2\|p.R19S\|nonsynonymous SNV [mutant] | 0.305 | 144 | 145 |
| 4 | >KIAA0319L\|p.E927Q\|nonsynonymous SNV [mutant] | 0.314 | 146 | 147 |
| 5 | >EFCAB14\|p.S166F\|nonsynonymous SNV [mutant] | −0.062 | 148 | 149 |
| 6 | >FOXD2\|p.S180C\|nonsynonymous SNV [mutant] | −0.31 | 150 | 151 |
| 7 | >COL11A1\|p.L377M\|nonsynonymous SNV [mutant] | −0.295 | 152 | 153 |
| 8 | >PHGDH\|p.T213A\|nonsynonymous SNV [mutant] | 0.448 | 154 | 155 |
| 9 | >HIST2H2BE\|p.N85S\|nonsynonymous SNV [mutant] | −0.748 | 156 | 157 |
| 10 | >LYSMD1\|p.T97P\|nonsynonymous SNV [mutant] | −0.519 | 158 | 159 |
| 11 | >NES\|p.R113Q\|nonsynonymous SNV [mutant] | −1.414 | 160 | 161 |
| 12 | >SLAMF1\|p.I306T\|nonsynonymous SNV [mutant] | −0.295 | 162 | 163 |
| 13 | >ILDR2\|p.S270C\|nonsynonymous SNV [mutant] | −0.148 | 164 | 165 |
| 14 | >TIPRL\|p.S177C\|nonsynonymous SNV [mutant] | 1.457 | 166 | 167 |
| 15 | >PRRC2C\|p.I444M\|nonsynonymous SNV [mutant] | −2.424 | 168 | 169 |
| 16 | >SEC16B\|p.R975S\|nonsynonymous SNV [mutant] | −0.657 | 170 | 171 |
| 17 | >SMG7\|p.E479K\|nonsynonymous SNV [mutant] | −0.076 | 172 | 173 |
| 18 | >PRG4\|p.P99Q\|nonsynonymous SNV [mutant] | −2.114 | 174 | 175 |
| 19 | >PTPRC\|p.Y1120S\|nonsynonymous SNV [mutant] | −1.567 | 176 | 177 |
| 20 | >PLXNA2\|p.R1042W\|nonsynonymous SNV [mutant] | −0.729 | 178 | 179 |
| 21 | >VASH2\|p.T120I\|nonsynonymous SNV [mutant] | 0.095 | 180 | 181 |
| 22 | >USH2A\|p.S1231I\|nonsynonymous SNV [mutant] | 0.714 | 182 | 183 |
| 23 | >DISC1\|p.E726Q\|nonsynonymous SNV [mutant] | −0.881 | 184 | 185 |
| 24 | >KIF26B\|p.H264D\|nonsynonymous SNV [mutant] | −0.724 | 186 | 187 |
| 25 | >ARMC4\|p.Q321K\|nonsynonymous SNV [mutant] | −1.324 | 188 | 189 |
| 26 | >FRMPD2\|p.Q91K\|nonsynonymous SNV [mutant] | −0.986 | 190 | 191 |
| 27 | >TET1\|p.E995K\|nonsynonymous SNV [mutant] | −0.829 | 192 | 193 |
| 28 | >AIFM2\|p.E219Q\|nonsynonymous SNV [mutant] | −0.848 | 194 | 195 |
| 29 | >PLCE1\|p.S61L\|nonsynonymous SNV [mutant] | 1.443 | 196 | 197 |
| 30 | >GPAM\|p.G444V\|nonsynonymous SNV [mutant] | −1.562 | 198 | 199 |
| 31 | >TECTB\|p.A132S\|nonsynonymous SNV [mutant] | −0.4 | 200 | 201 |
| 32 | >HABP2\|p.W356R\|nonsynonymous SNV [mutant] | 0.871 | 202 | 203 |
| 33 | >ADRB1\|p.E250K\|nonsynonymous SNV [mutant] | −0.248 | 204 | 205 |
| 34 | >DOCK1\|p.W103C\|nonsynonymous SNV [mutant] | −1.024 | 206 | 207 |
| 35 | >KRTAP5-3\|p.S166C\|nonsynonymous SNV [mutant] | 0.143 | 208 | 209 |
| 36 | >OR51S1\|p.A58V\|nonsynonymous SNV [mutant] | 0.624 | 210 | 211 |
| 37 | >DCDC5\|p.R345K\|nonsynonymous SNV [mutant] | 0.905 | 212 | 213 |
| 38 | >OR5M8\|p.E82Q\|nonsynonymous SNV [mutant] | −0.01 | 214 | 215 |
| 39 | >OR1S1\|p.G154V\|nonsynonymous SNV [mutant] | 1.243 | 216 | 217 |
| 40 | >MS4A6A\|p.S22C\|nonsynonymous SNV [mutant] | −0.305 | 218 | 219 |
| 41 | >MS4A4A\|p.W32L\|nonsynonymous SNV [mutant] | −0.01 | 220 | 221 |
| 42 | >MAP3K11\|p.S300C\|nonsynonymous SNV [mutant] | 0.362 | 222 | 223 |
| 43 | >PCNXL3\|p.V1269L\| nonsynonymous SNV [mutant] | 0.871 | 224 | 225 |
| 44 | >RCE1\|p.E22Q\|nonsynonymous SNV [mutant] | −0.343 | 226 | 227 |
| 45 | >CARNS1\|p.L351V\|nonsynonymous SNV [mutant] | 1.014 | 228 | 229 |
| 46 | >PDE2A\|p.D232H\|nonsynonymous SNV [mutant] | 0.357 | 230 | 231 |
| 47 | >MYO7A\|p.D2029Y\|nonsynonymous SNV [mutant] | −0.481 | 232 | 233 |
| 48 | >FAT3\|p.I3772M\|nonsynonymous SNV [mutant] | −0.186 | 234 | 235 |
| 49 | >FUT4\|p.E298Q\|nonsynonymous SNV [mutant] | −0.981 | 236 | 237 |
| 50 | >B3GAT1\|p.D122N\|nonsynonymous SNV [mutant] | −0.319 | 238 | 239 |
| 51 | >CACNA1C\|p.P1820T\|nonsynonymous SNV [mutant] | −1.352 | 240 | 241 |
| 52 | >SLC2A3\|p.G109E\|nonsynonymous SNV [mutant] | 1.052 | 242 | 243 |
| 53 | >SLCO1C1\|p.G396A\|nonsynonymous SNV [mutant] | 0.957 | 244 | 245 |
| 54 | >NELL2\|p.S596I\|nonsynonymous SNV [mutant] | −0.7 | 246 | 247 |
| 55 | >KMT2D\|p.E2866K\|nonsynonymous SNV [mutant] | −0.424 | 248 | 249 |
| 56 | >PAN2\|p.E630Q\|nonsynonymous SNV [mutant] | −0.49 | 250 | 251 |
| 57 | >LRIG3\|p.I341V\|nonsynonymous SNV [mutant] | 0.033 | 252 | 253 |
| 58 | >ZDHHC17\|p.D154N\|nonsynonymous SNV [mutant] | 0.181 | 254 | 255 |
| 59 | >OTOGL\|p.L43V\|nonsynonymous SNV [mutant] | −0.79 | 256 | 257 |
| 60 | >PPFIA2\|p.S16R\|nonsynonymous SNV [mutant] | −1.443 | 258 | 259 |
| 61 | >ALDH1L2\|p.K754N\|nonsynonymous SNV [mutant] | −0.367 | 260 | 261 |
| 62 | >ATP8A2\|p.E680Q\|nonsynonymous SNV [mutant] | 0.348 | 262 | 263 |
| 63 | >MTUS2\|p.W145R\|nonsynonymous SNV [mutant] | −0.681 | 264 | 265 |
| 64 | >MTUS2\|p.T550K\|nonsynonymous SNV [mutant] | −0.552 | 266 | 267 |
| 65 | >BRCA2\|p.K2750N\|nonsynonymous SNV [mutant] | 0.31 | 268 | 269 |
| 66 | >NBEA\|p.E2100Q\|nonsynonymous SNV [mutant] | −0.19 | 270 | 271 |
| 67 | >RAB20\|p.S52C\|nonsynonymous SNV [mutant] | −0.8 | 272 | 273 |
| 68 | >F7\|p.A429T\|nonsynonymous SNV [mutant] | 0.162 | 274 | 275 |
| 69 | >NPAS3\|p.G35R\|nonsynonymous SNV [mutant] | −1.71 | 276 | 277 |
| 70 | >DDX24\|p.T554K\|nonsynonymous SNV [mutant] | −0.695 | 278 | 279 |
| 71 | >DYNC1H1\|p.V2568I\|nonsynonymous SNV [mutant] | −0.257 | 280 | 281 |
| 72 | >KIF26A\|p.A254S\|nonsynonymous SNV [mutant] | 0.795 | 282 | 283 |
| 73 | >HERC2\|p.S319C\|nonsynonymous SNV [mutant] | −0.105 | 284 | 285 |
| 74 | >MTMR10\|p.E387K\|nonsynonymous SNV [mutant] | 0.076 | 286 | 287 |
| 75 | >ARHGAP11A\|p.E36D\|nonsynonymous SNV [mutant] | −1.067 | 288 | 289 |
| 76 | >SLC27A2\|p.Y500N\|nonsynonymous SNV [mutant] | −0.652 | 290 | 291 |
| 77 | >PRTG\|p.N908S\|nonsynonymous SNV [mutant] | −0.181 | 292 | 293 |
| 78 | >ALDH1A2\|p.S22L\|nonsynonymous SNV [mutant] | 0.49 | 294 | 295 |
| 79 | >CHTF18\|p.A858T\|nonsynonymous SNV [mutant] | −0.938 | 296 | 297 |
| 80 | >IFT140\|p.R1404W\|nonsynonymous SNV [mutant] | −0.49 | 298 | 299 |
| 81 | >SNX29\|p.D644E\|nonsynonymous SNV [mutant] | −0.743 | 300 | 301 |
| 82 | >EEF2K\|p.D425G\|nonsynonymous SNV [mutant] | −1.514 | 302 | 303 |
| 83 | >QPRT\|p.R102W\|nonsynonymous SNV [mutant] | 0.671 | 304 | 305 |
| 84 | >CD2BP2\|p.S49G\|nonsynonymous SNV [mutant] | −2.076 | 306 | 307 |
| 85 | >PMFBP1\|p.L835P\|nonsynonymous SNV [mutant] | −1.405 | 308 | 309 |
| 86 | >PLCG2\|p.S1192C\|nonsynonymous SNV [mutant] | −0.3 | 310 | 311 |
| 87 | >ADAD2\|p.G44A\|nonsynonymous SNV [mutant] | −0.238 | 312 | 313 |
| 88 | >ZNF469\|p.G680D\|nonsynonymous SNV [mutant] | −0.162 | 314 | 135 |
| 89 | >MYH13\|p.M80I\|nonsynonymous SNV [mutant] | −0.71 | 316 | 317 |
| 90 | >TRPV2\|p.Q199H\|nonsynonymous SNV [mutant] | −0.114 | 318 | 319 |
| 91 | >LRRC75A\|p.Q199E\|nonsynonymous SNV [mutant] | −1.243 | 320 | 321 |
| 92 | >ATXN7L3\|p.L249V\|nonsynonymous SNV [mutant] | −0.2 | 322 | 323 |
| 93 | >HOXB2\|p.P91Q\|nonsynonymous SNV [mutant] | −0.938 | 324 | 325 |
| 94 | >MPO\|p.F508L\|nonsynonymous SNV [mutant] | −0.676 | 326 | 327 |
| 95 | >TRIM37\|p.R192W\|nonsynonymous SNV [mutant] | 0.243 | 328 | 329 |
| 96 | >CHMP1B\|p.Q146H\|nonsynonymous SNV [mutant] | −0.29 | 330 | 331 |
| 97 | >SEH1L\|p.D118N\|nonsynonymous SNV [mutant] | −0.071 | 332 | 333 |
| 98 | >KCTD1\|p.S137L\|nonsynonymous SNV [mutant] | −0.548 | 334 | 335 |
| 99 | >EVI5L\|p.P715S\|nonsynonymous SNV [mutant] | 0.1 | 336 | 337 |
| 100 | >KEAP1\|p.E218K\|nonsynonymous SNV [mutant] | −0.371 | 338 | 339 |
| 101 | >MRI1\|p.G69E\|nonsynonymous SNV [mutant] | 1.338 | 340 | 341 |
| 102 | >ZNF257\|p.T304N\|nonsynonymous SNV [mutant] | −1.019 | 342 | 343 |
| 103 | >VSTM2B\|p.E161Q\|nonsynonymous SNV [mutant] | −0.414 | 344 | 345 |
| 104 | >DMKN\|p.D93H\|nonsynonymous SNV [mutant] | 0.224 | 346 | 347 |
| 105 | >BCKDHA\|p.E238K\|nonsynonymous SNV [mutant] | 0.086 | 348 | 349 |
| 106 | >CEACAM16\|p.S155R\|nonsynonymous SNV [mutant] | −0.752 | 350 | 351 |
| 107 | >NKPD1\|p.Q125E\|nonsynonymous SNV [mutant] | −0.338 | 352 | 353 |
| 108 | >EXOC3L2\|p.R39L\|nonsynonymous SNV [mutant] | −0.267 | 354 | 355 |
| 109 | >CA11\|p.Q282H\|nonsynonymous SNV [mutant] | −0.748 | 356 | 357 |
| 110 | >NLRP8\|p.R781S\|nonsynonymous SNV [mutant] | 0.11 | 358 | 3.59 |
| 111 | >ZNF470\|p.F462L\|nonsynonymous SNV [mutant] | −0.557 | 360 | 361 |
| 112 | >ZNF586\|p.R56T\|nonsynonymous SNV [mutant] | −0.314 | 362 | 363 |
| 113 | >ZSCAN1\|p.Q134P\|nonsynonymous SNV [mutant] | −0.419 | 364 | 365 |
| 114 | >TPO\|p.A90E\|nonsynonymous SNV [mutant] | −0.171 | 366 | 367 |
| 115 | >LTBP1\|p.V937L\|nonsynonymous SNV [mutant] | 0.748 | 368 | 369 |
| 116 | >AFF3\|p.E31Q\|nonsynonymous SNV [mutant] | −2.981 | 370 | 371 |
| 117 | >CKAP2L\|p.K30N\|nonsynonymous SNV [mutant] | −1.19 | 372 | 373 |
| 118 | >MYO7B\|p.A1791V\|nonsynonymous SNV [mutant] | −0.843 | 374 | 375 |
| 119 | >TANC1\|p.K906M\|nonsynonymous SNV [mutant] | 0.648 | 376 | 377 |
| 120 | >SLC4A10\|p.G309V\|nonsynonymous SNV [mutant] | 0.186 | 378 | 379 |
| 121 | >SCN2A\|p.S661C\|nonsynonymous SNV [mutant] | 0.824 | 380 | 381 |
| 122 | >SP9\|p.T14K\|nonsynonymous SNV [mutant] | 0.329 | 382 | 383 |
| 123 | >TTN\|p.C8217Y\|nonsynonymous SNV [mutant] | −0.695 | 384 | 385 |
| 124 | >HECW2\|p.D1350H\|nonsynonymous SNV [mutant] | −0.405 | 386 | 387 |
| 125 | >PARD3B\|p.Y789C\|nonsynonymous SNV [mutant] | −0.819 | 388 | 389 |
| 126 | >DIS3L2\|p.P77S\|nonsynonymous SNV [mutant] | 0.114 | 390 | 391 |
| 127 | >LZTS3\|p.E465K\|nonsynonymous SNV [mutant] | 0.081 | 392 | 393 |
| 128 | >KCNG1\|p.R205H\|nonsynonymous SNV [mutant] | −1.376 | 394 | 395 |
| 129 | >COL20A1\|p.N255D\|nonsynonymous SNV [mutant] | −0.119 | 396 | 397 |
| 130 | >BRWD1\|p.A2213V\|nonsynonymous SNV [mutant] | −0.586 | 398 | 399 |
| 131 | >DSCAM\|p.F271L\|nonsynonymous SNV [mutant] | −0.043 | 400 | 401 |
| 132 | >KRTAP10-4\|p.S221T\|nonsynonymous SNV [mutant] | 0.048 | 402 | 403 |
| 133 | >NEFH\|p.V446A\|nonsynonymous SNV [mutant] | −0.729 | 404 | 405 |
| 134 | >SFI1\|p.T128A\|nonsynonymous SNV [mutant] | −0.21 | 406 | 407 |
| 135 | >POLR3H\|p.R149C\|nonsynonymous SNV [mutant] | −0.062 | 408 | 409 |
| 136 | >STAB1\|p.S681R\|nonsynonymous SNV [mutant] | −0.352 | 410 | 411 |
| 137 | >SLC25A26\|p.S82L\|nonsynonymous SNV [mutant] | −0.219 | 412 | 413 |
| 138 | >EPHA6\|p.V196L\|nonsynonymous SNV [mutant] | −0.462 | 414 | 415 |
| 139 | >PLXNA1\|p.E607K\|nonsynonymous SNV [mutant] | −0.619 | 416 | 417 |
| 140 | >DNAJC13\|p.K514I\|nonsynonymous SNV [mutant] | −0.433 | 418 | 419 |
| 141 | >ESYT3\|p.K496N\|nonsynonymous SNV [mutant] | −0.905 | 420 | 421 |
| 142 | >GPR149\|p.R145G\|nonsynonymous SNV [mutant] | 0.09 | 422 | 423 |
| 143 | >PDCD10\|p.E68Q\|nonsynonymous SNV [mutant] | −0.033 | 424 | 425 |
| 144 | >MECOM\|p.A78T\|nonsynonymous SNV [mutant] | −0.39 | 426 | 427 |
| 145 | >KIAA0226\|p.R150K\|nonsynonymous SNV [mutant] | −0.176 | 428 | 429 |
| 146 | >MSX1\|p.S92L\|nonsynonymous SNV [mutant] | 0.043 | 430 | 431 |
| 147 | >LIMCH1\|p.Q60H\|nonsynonymous SNV [mutant] | −0.648 | 432 | 433 |
| 148 | >PTPN13\|p.Q2276H\|nonsynonymous SNV [mutant] | 0 | 434 | 435 |
| 149 | >PDHA2\|p.C179Y\|nonsynonymous SNV [mutant] | 0.2 | 436 | 437 |
| 150 | >EXOSC9\|p.L266F\|nonsynonymous SNV [mutant] | −0.11 | 438 | 439 |
| 151 | >TBC1D9\|p.E837Q\|nonsynonymous SNV [mutant] | −0.005 | 440 | 441 |
| 152 | >FGG\|p.G294E\|nonsynonymous SNV [mutant] | −0.757 | 442 | 443 |
| 153 | >SLC9A3\|p.E821Q\|nonsynonymous SNV [mutant] | −0.662 | 444 | 445 |
| 154 | >NSUN2\|p.F48L\|nonsynonymous SNV [mutant] | −0.6 | 446 | 447 |
| 155 | >SPEF2\|p.F1436S\|nonsynonymous SNV [mutant] | −0.495 | 448 | 449 |
| 156 | >ITGA2\|p.P43T\|nonsynonymous SNV [mutant] | −0.162 | 450 | 451 |
| 157 | >IPO11\|p.N30S\|nonsynonymous SNV [mutant] | 0.076 | 452 | 453 |
| 158 | >NR2F1\|p.V380M\|nonsynonymous SNV [mutant] | 0.833 | 454 | 455 |
| 159 | >SLCO4C1\|p.K663N\|nonsynonymous SNV [mutant] | 0.781 | 456 | 457 |
| 160 | >WDR55\|p.F237L\|nonsynonymous SNV [mutant] | −0.114 | 458 | 459 |
| 161 | >PCDHA9\|p.T662S\|nonsynonymous SNV [mutant] | 0.286 | 460 | 461 |
| 162 | >PCDHGA12\|p.K590M\|nonsynonymous SNV | −0.138 | 462 | 463 |
| | [mutant]RSAEPGYLVTMVVAVDRDSGQ | | | |
| 163 | >SLC6A7\|p.D151H\|nonsynonymous SNV [mutant] | 0.162 | 464 | 465 |
| 164 | >TCOF1\|p.P566S\|nonsynonymous SNV [mutant] | −0.048 | 466 | 467 |
| 165 | >LCP2\|p.P138H\|nonsynonymous SNV [mutant] | −2.005 | 468 | 469 |
| 166 | >KCNIP1\|p.I19M\|nonsynonymous SNV [mutant] | 0.448 | 470 | 471 |
| 167 | >BTN3A1\|p.A186S\|nonsynonymous SNV [mutant] | −0.39 | 472 | 473 |
| 168 | >ZBTB12\|p.C266S\|nonsynonymous SNV [mutant] | −0.348 | 474 | 475 |
| 169 | >CYP21A2\|p.E295K\|nonsynonymous SNV [mutant] | −1.167 | 476 | 477 |
| 170 | >TREM2\|p.E202V\|nonsynonymous SNV [mutant] | 0.633 | 478 | 479 |
| 171 | >TTK\|p.L309F\|nonsynonymous SNV [mutant] | −1.219 | 480 | 481 |
| 172 | >SYNCRIP\|p.D284H\|nonsynonymous SNV [mutant] | −0.667 | 482 | 483 |
| 173 | >HS3ST5\|p.R82G\|nonsynonymous SNV [mutant] | −1.048 | 484 | 485 |
| 174 | >RFX6\|p.Y802C\|nonsynonymous SNV [mutant] | −1.086 | 486 | 487 |
| 175 | >SYNE1\|p.T5594A\|nonsynonymous SNV [mutant] | −0.438 | 488 | 489 |
| 176 | >CYP2W1\|p.R328H\|nonsynonymous SNV [mutant] | −0.729 | 490 | 491 |
| 177 | >GNAT3\|p.E216Q\|nonsynonymous SNV [mutant] | 0.433 | 492 | 493 |
| 178 | >SEMA3C\|p.Y141C\|nonsynonymous SNV [mutant] | −0.752 | 494 | 495 |
| 179 | >PCLO\|p.E1590D\|nonsynonymous SNV [mutant] | −1.938 | 496 | 497 |
| 180 | >SAMD9\|p.I635V\|nonsynonymous SNV [mutant] | −0.114 | 498 | 499 |
| 181 | >COG5\|p.A54G\|nonsynonymous SNV [mutant] | 0.11 | 500 | 501 |
| 182 | >CBLL1\|p.S471F\|nonsynonymous SNV [mutant] | −1.19 | 502 | 503 |
| 183 | >FOXP2\|p.S139C\|nonsynonymous SNV [mutant] | −0.129 | 504 | 505 |
| 184 | >MDFIC\|p.L78F\|nonsynonymous SNV [mutant] | −0.786 | 506 | 507 |
| 185 | >IMPDH1\|p.P138L\|nonsynonymous SNV [mutant] | −0.148 | 508 | 509 |
| 186 | >TRIM24\|p.T772S\|nonsynonymous SNV [mutant] | −0.376 | 510 | 511 |
| 187 | >CASP2\|p.A338S\|nonsynonymous SNV [mutant] | −1.533 | 512 | 513 |
| 188 | >ASIC3\|p.L531Q\|nonsynonymous SNV [mutant] | −0.41 | 514 | 515 |
| 189 | >INSIG1\|p.L126F\|nonsynonymous SNV [mutant] | −0.09 | 516 | 517 |
| 190 | >TNKS\|p.I1189F\|nonsynonymous SNV [mutant] | −0.205 | 518 | 519 |
| 191 | >CHD7\|p.Q1704E\|nonsynonymous SNV [mutant] | −0.748 | 520 | 521 |
| 192 | >ZFHX4\|p.T3413A\|nonsynonymous SNV [mutant] | −0.381 | 522 | 523 |
| 193 | >CNGB3\|p.L227H\|nonsynonymous SNV [mutant] | 1.152 | 524 | 525 |
| 194 | >HAS2\|p.M118V\|nonsynonymous SNV [mutant] | 0.11 | 526 | 527 |
| 195 | >LRRC6\|p.D453N\|nonsynonymous SNV [mutant] | −1.286 | 528 | 529 |
| 196 | >PLEC\|p.E1404D\|nonsynonymous SNV [mutant] | −1.4 | 530 | 531 |
| 197 | >RORB\|p.R372G\|nonsynonymous SNV [mutant] | 0.267 | 532 | 533 |
| 198 | >SPATA31C2\|p.A1072E\|nonsynonymous SNV [mutant] | −1.09 | 534 | 535 |
| 199 | >SVEP1\|p.R146S\|nonsynonymous SNV [mutant] | −0.571 | 536 | 537 |
| 200 | >REXO4\|p.T241S\|nonsynonymous SNV [mutant] | 0.581 | 538 | 539 |
| 201 | >RPS6KA3\|p.P617L\|nonsynonymous SNV [mutant] | −0.081 | 540 | 541 |
| 202 | >SMC1A\|p.K402Q\|nonsynonymous SNV [mutant] | −1.81 | 542 | 543 |
| 203 | >MSN\|p.K211N\|nonsynonymous SNV [mutant] | −0.395 | 544 | 545 |
| 204 | >TEX11\|p.S163C\|nonsynonymous SNV [mutant] | −0.148 | 546 | 547 |
| 205 | >SERPINA7\|p.K290R\|nonsynonymous SNV [mutant] | −0.695 | 548 | 549 |
| 206 | >IL9R\|p.V287E\|nonsynonymous SNV [mutant] | −1.505 | 550 | 551 |

### Reagents

The following reagents were used to test the lung permutation constructs:
Bacteria: Lmdda constructs tagged grown overnight in BHI
Cell lines: DC2.4
2% trypsin in HBSS
RPMI 10% FBS glutamax
FACS Buffer (PBS 2% FBS)
Cell counting solution
Gentamicin antibiotic
25D-APC conjugated antibody 100×

### Harvesting Antigen Presenting Cells

In some experiments, murine dendritic DC2.4 cells were stimulated with 20 ng/mL recombinant mouse IFN gamma for 48 hours. Media was removed and collected into two 50 mL conical tubes per flask. A volume of 10 ml 2% trypsin HBSS solution is added to the flask to remove residual FBS and was decanted into the two 50 mL collection tubes equally (5 mL: each). A volume of 10 mL 2% trypsin HBSS solution was added to the flask to coat, and adherence was checked under a microscope, and a 5 min incubation followed at 37° C. The suspension was collected into the two 50-mL collection tubes (5 mL each) and spun for 5 minutes at 1200 rpm. The supernatant was discarded, and the pellet was resuspended in tube 1 with 25 mL RPMI 10% FBS glutamax solution. This was then decanted into the second collection tube to combine the two tubes into one.

Tubes (1.5 mL) were then labeled for counting. A volume of 135 uL counting solution and a volume of 15 uL cells was added, and the cells were incubated for 2 minutes at room temperature. The DC2.4 cells were then set up for infection in 24-well plates. The 24-well plates were incubated overnight at 37° C. (5% CO₂). The plates were then spun for 5 seconds at 2000 rpm, supernatant was removed, and 1 mL fresh c-RPMI was added to each well.

### Infection

The cells were then infected with Lmdda-PSA-Survivin-tag expressing vectors (grown overnight dry 37). Total Lmdda-neo construct cfu were 1×10⁹/mL The Lmdda was spun down in 1.5 mL and resuspended in 1 mL room temperature RPMI-10% FBS media. A volume of the Lmdda was added to the DC2.4 wells to reach the correct MOI for 2×10⁶ cells (MOI: 10=20 uL Lmdda). The plate was then spun at 1200 rpm for 15 minutes and placed in an incubator at 37° C. with 5% CO₂ for a four hour infection. To stop Lmdda killing of the cells, 10 ug/mL gentamicin was added after 1 hour of the incubation.

Staining with 25D-APC (SIINFEKL) and Flow Cytometry

After four hours of infection, the plate was spun for 30 seconds at 2000 rpm, and the supernatant was discarded. To block the cells were resuspended in 200 uL 2.4G2 and transferred to a 96-well plate for 10 minute on ice. The cells were washed with FACS buffer (PBS+2% FBS). Staining master mix was then added, and the cells were vortexed and placed on ice for 20 minutes. The cells were then washed with FACS buffer and resuspended in approximately 300 uL FACS buffer (depending on size of pellet/cell number). The samples were then run on the flow cytometer for detection of 25D-APC.

### Experiment 1

TABLE 10
Samples Tested for Detection of 25D-APC.

| Sample # | Lung Construct | Order of Neo-Epitopes |
|---|---|---|
| 1 | SVN-tag | |
| 2 | SVN-no | |
| 3 | PSMA | |
| 4 | R1 | 160, 171, 19, 129, 96, 127, 115, 42, 131, 196, 118, 36, 113, 56, 30, |
| | (SEQ ID NOS: 552, 553) | 84, 80, 32, 200, 21 |
| | (DNA sequence, peptide | |
| | sequence) | |
| 5 | R2 | 178, 182, 148, 3, 106, 173, 187, 52, 168, 160, 65, 119, 181, 16, 23, |
| | (SEQ ID NOS: 554, 555) | 156, 31, 122, 42, 54 |
| 6 | R5 | 48, 178, 65, 165, 50, 185, 55, 119, 84, 180, 98, 35, 166, 110, 176, 49, |
| | (SEQ ID NOS: 556, 557) | 41, 163, 20, 152 |
| 7 | R6 | 105, 66, 65, 69, 152, 3, 46, 91, 79, 114, 58, 73, 125, 8, 163, 156, 31, |
| | (SEQ ID NOS: 558, 559) | 44, 7, 103 |
| 8 | R7 opt | 158, 176, 36, 150, 56, 15, 120, 40, 167, 29, 187, 5, 102, 1, 91, 44, 22, |
| | (SEQ ID NOS: 560, 561) | 108, 193, 68 |
| 9 | R8 | 38, 31, 89, 22, 152, 169, 82, 144, 184, 49, 30, 5, 187, 94, 95, 167, |
| | (SEQ ID NOS: 562, 563) | 194, 118, 136, 9 |
| 10 | R9 | 65, 144, 127, 194, 94, 186, 67, 32, 117, 7, 92, 165, 179, 8, 10, 129, |
| | (SEQ ID NOS: 564, 565) | 145, 130, 104, 60 |
| 11 | R10 opt | 8, 116, 29, 185, 194, 47, 95, 101, 51, 21, 195, 162, 123, 10, 20, 63, |
| | (SEQ ID NOS: 566, 567) | 117, 2, 184, 130 |
| 12 | R11 | 184, 129, 92, 154, 159, 167, 40, 67, 113, 189, 77, 18, 150, 87, 196, |
| | (SEQ ID NOS: 568, 569) | 31, 13, 100, 15, 22 |
| 13 | R 14 opt | 69, 155, 100, 1, 81, 139, 78, 154, 49, 38, 52, 5, 133, 39, 42, 161, 83, |
| | (SEQ ID NOS: 570, 571) | 166, 163, 157 |
| 14 | R18 | 155, 75, 35, 135, 112, 19, 59, 160, 14, 16, 103, 120, 127, 31, 157, |
| | (SEQ ID NOS: 572, 573) | 167, 12, 52, 70, 177 |
| 15 | R19 | 94, 57, 174, 149, 117, 24, 170, 20, 12, 176, 146, 127, 108, 53, 46, 27, |
| | (SEQ ID NOS: 574, 575) | 157, 29, 59, 130 |
| 16 | R20 | 128, 97, 122, 170, 18, 106, 37, 100, 49, 132, 185, 19, 59, 167, 188, |
| | (SEQ ID NOS: 576, 577) | 115, 127, 64, 169, 117 |
| 17 | rM 2 | LCMV, 14, 169, 28, VSV, 144, 195, 189, PSA, 90, 101, 155, VV, |
| | (SEQ ID NOS: 578, 579) | 102, 43, 26, IAV, 82, 31, 95 |
| 18 | 1-20 | 1-20 |
| | (SEQ ID NOS: 580, 581) | |
| 19 | 21-40 opt | 21-40 except #39 moved to 4^{th} position |
| | (SEQ ID NOS: 582, 583) | |
| 20 | 41-60 | 41-60 |
| | (SEQ ID NOS: 584, 585) | |
| 21 | 81-100 | 81-100 |
| | (SEQ ID NOS: 586, 587) | |
| 22 | 101-120 | 101-120 |
| | (SEQ ID NOS: 588, 589) | |
| 23 | 121-140 | 121-140 |
| | (SEQ ID NOS: 590, 591) | |
| 24 | 141-160 | 141-160 |
| | (SEQ ID NOS: 592, 593) | |
| 25 | 61-80 H (SEQ ID NO: 618) | 61-80 |
| | (peptide sequence only) | |
| 26 | 81-100 H (SEQ ID NO: 587) | 81-100 |
| | (peptide sequence only) | |
| 27 | 101-120 H (SEQ ID NO: | 101-120 |
| | 589) (peptide sequence only) | |
| 28 | 121-140 H (SEQ ID NO: | 121-140 |
| | 591) (peptide sequence only) | |
| 29 | 141-160 H (SEQ ID NO: | 141-160 |
| | 593) (peptide sequence only) | |
| 30 | No Infection | |
| 31 | Unstained | |

An “H” at the end of the construct (such as “121-140 H”) indicates that the peptide sequence is identical to the construct lacking the H, but the underlying nucleotide sequence which resulted in the same peptide sequence was modified.

TABLE 11
Detection of 25D-APC.

| | | | | MFI Ratio | Percent Ratio |
|---|---|---|---|---|---|
| Sample | Lung | live \| Mean | Percent | (Exp-Background)/ | (Exp-Background)/ |
| # | Construct | (R 670-20) | Positive | (SVN-background) | (SVN-background) |
| 1 | SVN-tag | 605 Positive Ctrl | 16.7 | — | — |
| 2 | SVN-no | 115 Background | .30 | — | — |
| 3 | PSMA | 253 PSMA Ctrl | 5.13 | 0.281633 | 0.294512 |
| 4 | R1 | 350 | 8.61 | 0.479592 | 0.506707 |
| 5 | R2 | 309 | 6.81 | 0.395918 | 0.396951 |
| 6 | R5 | 162 | 1.13 | 0.095918 | 0.05061 |
| 7 | R6 | 286 | 6.59 | 0.34898 | 0.383537 |
| 8 | R7 opt | 152 | .39 | 0.07551 | 0.005488 |
| 9 | R8 | 155 | .60 | 0.081633 | 0.018293 |
| 10 | R9 | 788 | 18.9 | 1.373469 | 1.134146 |
| 11 | R10 opt | 182 | .99 | 0.136735 | 0.042073 |
| 12 | R11 | 222 | 2.40 | 0.218367 | 0.128049 |
| 13 | R 14 opt | 112 | .09 | −0.00612 | −0.0128 |
| 14 | R18 | 117 | .26 | 0.004082 | −0.00244 |
| 15 | R19 | 109 | .1 | −0.01224 | −0.0122 |
| 16 | R20 | 203 | 3.3 | 0.179592 | 0.182927 |
| 17 | rM 2 | 116 | .15 | 0.002041 | −0.00915 |
| 18 | 1-20 | 134 | .27 | 0.038776 | −0.00183 |
| 19 | 21-40 opt | 129 | .26 | 0.028571 | −0.00244 |
| 20 | 41-60 | 299 | 6.52 | 0.37551 | 0.379268 |
| 21 | 81-100 | 187 | 1.51 | 0.146939 | 0.07378 |
| 22 | 101-120 | 428 | 10 | 0.638776 | 0.591463 |
| 23 | 121-140 | 368 | 9.26 | 0.516327 | 0.546341 |
| 24 | 141-160 | 144 | .61 | 0.059184 | 0.018902 |
| 25 | 61-80 H | 312 | 7.68 | 0.402041 | 0.45 |
| 26 | 81-100 H | 151 | .59 | 0.073469 | 0.017683 |
| 27 | 101-120 H | 363 | 8.05 | 0.506122 | 0.472561 |
| 28 | 121-140 H | 281 | 5.33 | 0.338776 | 0.306707 |
| 29 | 141-160 H | 204 | 2.04 | 0.181633 | 0.106098 |
| 30 | No Infection | 102 | .07 | −0.02653 | −0.01402 |
| 31 | Unstained | 63.8 | .04 | −0.10449 | −0.01585 |

Detection of the C-terminal SIINFEKL tag with the 25D-APC conjugated antibody is shown in Table 11. As indicated in Table 11, the SVN-tag and PSMA-tag positive controls showed high levels of positive staining, whereas the SVN-no tag, the no infection, and the unstained negative controls were below the limit of detection. Similarly, samples 4-7, 10, 12, 16, 20-23, 25, and 27-29 showed high levels of positive staining. This demonstrates confirmation that the neo-antigens express and secrete in antigen-presenting cells upon infection.

### Experiment 2

The above was repeated in a second experiment with additional lung neo-epitope constructs, as indicated in Table 12. In this experiment, the tag was moved to different locations within the lung constructs.

TABLE 12
Samples Tested for Detection of 25D-APC.

| Sample | | |
|---|---|---|
| # | Lung Construct | Explanation of Sample |
| 1 | SVN | |
| 2 | No Tag | |
| 3 | 21416 | 121-135-SIINFEKL-136-140-6xHIS |
| | (SEQ ID NOS: 594, | |
| | 595) (DNA sequence, | |
| | peptide sequence) | |
| 4 | 21417 | SIINFEKL-random8 1-20-6xHIS |
| | (SEQ ID NOS: 596, | |
| | 597) | |
| 5 | 21419 | random8 1-10-SIINFEKL-11-20-6xHIS |
| | (SEQ ID NOS: 598, | |
| | 599) | |
| 6 | 20724 (SEQ ID NO: | 2712 1-20-tags H |
| | 581) (peptide sequence | |
| | only) | |
| 7 | 21412 | 41-55-SIINFEKL-56-60-6xHIS |
| | (SEQ ID NOS: 600, | |
| | 601) | |
| 8 | 20726 (SEQ ID NO: | 2712 41-60-tags H |
| | 585) (peptide sequence | |
| | only) | |
| 9 | 20725 (SEQ ID NO: | 2712 21-40opt-tags H |
| | 583) (peptide sequence | |
| | only) | |
| 10 | 21411 | 41-50-SIINFEKL-51-60-6xHIS |
| | (SEQ ID NOS: 602, | |
| | 603) | |
| 11 | 21409 | SIINFEKL-41 -60-6xHIS |
| | (SEQ ID NOS: 604, | |
| | 605) | |
| 12 | 21420 | random8 1-15-SIINFEKL-16-20-6xHIS |
| | (SEQ ID NOS: 606, | |
| | 607) | |
| 13 | 21418 (SEQ ID NOS: | 2712 random8 1-5-SIINFEKL-6-20-HIS |
| | 619, 620) | |
| 14 | 19411 (SEQ ID NOS: | 2712 random16opt-tags |
| | 621, 622) | |
| 15 | 41-60 (SEQ ID NOS: | 41-60 |
| | 584, 585) | |
| 16 | no infection | |
| 17 | unstained | |

TABLE 13
Detection of 25D-APC.

| | | | | MFI Ratio | Percent Ratio |
|---|---|---|---|---|---|
| Sample | Lung | live \| Mean | Percent | (Exp-Background)/ | (Exp-Background)/ |
| # | Construct | (R 670-20) | Positive | (SVN-background) | (SVN-background) |
| 1 | SVN | 325 | 2.46 | — | — |
| 2 | No Tag | 239 | 0.29 | — | — |
| 3 | 21416 | 285 | 1.16 | 0.534884 | 0.400922 |
| 4 | 21417 | 248 | 0.4 | 0.104651 | 0.050691 |
| 5 | 21419 | 223 | 0.23 | −0.18605 | −0.02765 |
| 6 | 20724 | 236 | 0.3 | −0.03488 | 0.004608 |
| 7 | 21412 | 255 | 0.79 | 0.186047 | 0.230415 |
| 8 | 20726 | 265 | 1.12 | 0.302326 | 0.382488 |
| 9 | 20725 | 218 | 0.24 | −0.24419 | −0.02304 |
| 10 | 21411 | 269 | 0.45 | 0.348837 | 0.073733 |
| 11 | 21409 | 240 | 0.32 | 0.011628 | 0.013825 |
| 12 | 21420 | 231 | 0.16 | −0.09302 | −0.05991 |
| 13 | 21918 | 222 | 0.31 | −0.19767 | 0.009217 |
| 14 | 19411 | 228 | 0.25 | −0.12791 | −0.01843 |
| 15 | 41-60 | 232 | 0.46 | −0.0814 | 0.078341 |
| 16 | no infection | 263 | 0.81 | 0.27907 | 0.239631 |
| 17 | unstained | 78.4 | 0.02 | −1.86744 | −0.12442 |

Detection of the C-terminal SIINFEKL tag with the 25D-APC conjugated antibody is shown in Table 13. As indicated in Table 13, the SVN-tag positive control showed high levels of positive staining, whereas the no tag, the no infection, and the unstained negative controls were below the limit of detection. Similarly, samples 3, 7, and 8 showed high levels of positive staining. This demonstrates confirmation that the neo-antigens express and secrete in antigen-presenting cells upon infection.

### Experiment 3

The above was repeated in a third experiment with additional lung constructs, as indicated in Table 14. In these lung constructs, the tag was moved to different locations in the construct.

TABLE 14
Samples Tested for Detection of 25D-APC.

| Sample # | Lung Construct | Order of Neo-Epitopes and Tags |
|---|---|---|
| 1 | PSA Survivin | |
| 2 | Minigene | |
| 3 | No Tag | |
| 4 | 21409 | SIINFEKL-41-60-6xHIS |
| | (SEQ ID NOS: 604, | |
| | 605) (DNA sequence, | |
| | peptide sequence) | |
| 5 | 21410 | 41-45-SIINFEKL-46-60-6xHIS |
| | (SEQ ID NOS: 608, | |
| | 609) | |
| 6 | 21411 | 41-50-SIINFEKL-51-60-6xHIS |
| | (SEQ ID NOS: 602, | |
| | 603) | |
| 7 | 21412 | 41-55-SIINFEKL-56-60-6xHIS |
| | (SEQ ID NOS: 600, | |
| | 601) | |
| 8 | 21414 | 121-125-SIINFEKL-126-140-6xHIS |
| | (SEQ ID NOS: 610, | |
| | 611) | |
| 9 | 21415 | 121-130-SIINFEKL-131-140-6xHIS |
| | (SEQ ID NOS: 612, | |
| | 613) | |
| 10 | 21416 | 121-135-SIINFEKL-136-140-6xHIS |
| | (SEQ ID NOS: 595, | |
| | 595) | |
| 11 | 41-60 | 41-60-SIINFEKL-6xHIS |
| | (SEQ ID NOS: 614, | |
| | 615) | |
| 12 | 121-140 | 121-140-SIINFEKL-6xHIS |
| | (SEQ ID NOS: 616, | |
| | 618) | |
| 13 | no inf | |

TABLE 15
Detection of 25D-APC.

| | | | Percent Ratio | | MFI Ratio |
|---|---|---|---|---|---|
| Sample | Lung | Percent | (Exp-Background)/ | | (Exp-Background)/ |
| # | Construct | Positive | (SVN-background) | MFI | (SVN-background) |
| 1 | PSA | 30.9 | | 1392 | |
| | Survivin | | | | |
| 2 | Minigene | 37.4 | | 2350 | |
| 3 | No Tag | 1.62 | | 424 | |
| 4 | 21409 | 6.85 | 0.17862 | 553 | 0.133264 |
| 5 | 21410 | 1.58 | −0.00137 | 422 | −0.00207 |
| 6 | 21411 | 5.92 | 0.146858 | 507 | 0.085744 |
| 7 | 21412 | 11.2 | 0.327186 | 692 | 0.27686 |
| 8 | 21414 | 4.95 | 0.11373 | 536 | 0.115702 |
| 9 | 21415 | 13.5 | 0.405738 | 741 | 0.327479 |
| 10 | 21416 | 4.96 | 0.114071 | 538 | 0.117769 |
| 11 | 41-60 | 11 | 0.320355 | 678 | 0.262397 |
| 12 | 121-140 | 12.2 | 0.361339 | 709 | 0.294421 |
| 13 | no inf | 0.01 | −0.05499 | 140 | −0.29339 |

Detection of the C-terminal SIINFEKL tag with the 25D-APC conjugated antibody is shown in Table 15. As indicated in Table 15, the PSA Survivin and the Minigene positive controls showed high levels of positive staining, whereas the no tag and the no infection negative controls were below the limit of detection. Similarly, samples 4 and 5-12 showed high levels of positive staining. This demonstrates confirmation that the neo-antigens express and secrete in antigen-presenting cells upon infection.

FIG. 45 shows surface K^{b}-SIINFEKL on DC2.4 cells infected with Lm constructs with SIINFEKL at various positions. The graph depicts a summary of the raw 25D data, depicting that the SIINFEKL tag identifies a secreted neo-epitope whether SIINFEKL is located at the C-terminus, the N-terminus, or in between. The last five bars correspond with the following constructs: 2712 SIINFEKL-121-140-6×HIS; 2712 121-125-SIINFEKL-126-140-6×HIS; 2712 121-130-SIINFEKL-131-140-6×HIS; 2712 121-135-SIINFEKL-136-140-6×HIS; and 2712 121-140-SIINFEKL-6×HIS, respectively.

### Experiment 4

The above was repeated in a fourth experiment with additional Lmdda constructs, as indicated in Table 16.

TABLE 16
Samples Tested for Detection of 25D-APC.

| | Lung | | |
|---|---|---|---|
| Sample # | Construct | Strain Name | Neo-Epitopes from N-term to C-term |
| 1 | SVN | | |
| 2 | No Tag | | |
| 3 | 599 | 1-5 | 1-5 |
| | | (SEQ ID NOS: 623, 624) | |
| | | (DNA sequence, peptide | |
| | | sequence) | |
| 4 | 600 | 6-10 | 6-10 |
| | | (SEQ ID NOS: 625, 626) | |
| 5 | 601 | 11-15 | 11-15 |
| | | (SEQ ID NOS: 627, 628) | |
| 6 | 605 | 1-20r1 11-20 | 13, 11, 9, 7, 20, 5, 2, 4, 3, 12 |
| | | (SEQ ID NOS: 629, 630) | |
| 7 | 606 | 1-20r1 1-5 | 10, 19, 16, 17, 15 |
| | | (SEQ ID NOS: 631, 632) | |
| 8 | 607 | 1-20r1 6-10 | 8, 6, 18, 14, 1 |
| | | (SEQ ID NOS: 633, 634) | |
| 9 | 608 | 1-20r1 11-15 | 13, 11, 9, 7, 20 |
| | | (SEQ ID NOS: 635, 636) | |
| 10 | 609 | 1-20r1 16-20 | 5, 2, 4, 3, 12 |
| | | (SEQ ID NOS: 637, 638) | |
| 11 | 614 | 1-20r2 6-10 | 18, 7, 16, 17, 6 |
| | | (SEQ ID NOS: 639, 640) | |
| 12 | 615 | 1-20r2 11-15 | 20, 12, 4, 8, 2 |
| | | (SEQ ID NOS: 641, 642) | |
| 13 | 619 | 21-40 1-5 | 21-25 |
| | | (SEQ ID NOS: 643, 644) | |
| 14 | 620 | 21-40 6-10 | 26-30 |
| | | (SEQ ID NOS: 645, 646) | |
| 15 | 622 | 21-40 16-20 | 36-40 |
| | | (SEQ ID NOS: 647, 648) | |
| 16 | 623 | 21-40r1 | 38, 22, 31, 36, 35, 24, 32, 33, 29, 39, 30, 28, 34, |
| | | (SEQ ID NOS: 649, 650) | 37, 21, 27, 25, 40, 26, 23 |
| 17 | 624 | 21-40r1 1-10 | 38, 22, 31, 36, 35, 24, 32, 33, 29, 39 |
| | | (SEQ ID NOS: 651, 652) | |
| 18 | 625 | 21-40r1 11-20 | 30, 28, 34, 37, 21, 27, 25, 40, 26, 23 |
| | | (SEQ ID NOS: 653, 654) | |
| 19 | 626 | 21-40r1 1-5 | 38, 22, 31, 36, 35 |
| | | (SEQ ID NOS: 655, 656) | |
| 20 | 627 | 21-40r1 6-10 | 24, 32, 33, 29, 39 |
| | | (SEQ ID NOS: 657, 658) | |
| 21 | 628 | 21-40r1 11-15 | 30, 28, 34, 37, 21 |
| | | (SEQ ID NOS: 659, 660) | |
| 22 | 629 | 21-40r1 16-20 | 5, 2, 4, 3, 12 |
| | | (SEQ ID NOS: 661, 662) | |
| 23 | 632 | 21-40r2 11-20 | 38, 22, 25, 27, 33, 26, 31, 24, 40, 29 |
| | | (SEQ ID NOS: 663, 664) | |
| 24 | 634 | 21-40r2 6-10 | 35, 21, 28, 34, 36 |
| | | (SEQ ID NOS: 665, 666) | |
| 25 | 398 | 2712 random3 | 71, 45, 89, 122, 31, 199, 95, 131, 35, 192, 154, |
| | | (SEQ ID NOS: 667, 668) | 136, 185, 124, 194, 73, 150, 159, 93, 190 |
| 26 | 412 | 2712 random17 | 64, 156, 93, 179, 187, 119, 90, 55, 9, 14, 153, 59, |
| | | (SEQ ID NOS: 669, 670) | 78, 151, 107, 170, 134, 148, 97, 29 |
| 27 | 413 | SIINFEKL 121-140-6xHIS | 121-140 |
| | | (SEQ ID NOS: 671, 672) | |
| 28 | 602 | 16-20 | 16-20 |
| | | (SEQ ID NOS: 673, 674) | |
| 29 | 603 | 1-20r1 | 10, 19, 16, 17, 15, 8, 6, 18, 14, 1, 13, 11, 9, 7, 20, |
| | | (SEQ ID NOS: 675, 676) | 5, 2, 4, 3, 12 |
| 30 | 604 | 1-20r1 1-10 | 10, 19, 16, 17, 15, 8, 6, 18, 14, 1 |
| | | (SEQ ID NOS: 677, 678) | |
| 31 | ISG15 | | |

TABLE 17
Detection of 25D-APC.

| | | | | Percent Ratio |
|---|---|---|---|---|
| Sample | Lung | | Percent | (Exp-Background)/ |
| # | Construct | Strain Name | Positive | (SVN-background) |
| 1 | SVN | | 14.0% | |
| 2 | No Tag | | 1.43% | |
| 3 | 599 | 1-5 | 9.56% | 0.646778 |
| 4 | 600 | 6-10 | 12.7% | 0.896579 |
| 5 | 601 | 11-15 | 4.67% | 0.257757 |
| 6 | 605 | 1-20r1 11-20 | 15.5% | 1.119332 |
| 7 | 606 | 1-20r1 1-5 | 15.1% | 1.08751 |
| 8 | 607 | 1-20r1 6-10 | 6.50% | 0.403341 |
| 9 | 608 | 1-20r1 11-15 | 6.51% | 0.404137 |
| 10 | 609 | 1-20r1 16-20 | 15.0% | 1.079554 |
| 11 | 614 | 1-20r2 6-10 | 7.49% | 0.4821 |
| 12 | 615 | 1-20r2 11-15 | 8.88% | 0.592681 |
| 13 | 619 | 21-40 1-5 | 0.51% | −0.07319 |
| 14 | 620 | 21-40 6-10 | 0.47% | −0.07637 |
| 15 | 622 | 21-40 16-20 | 5.54% | 0.326969 |
| 16 | 623 | 21-40r1 | 0.88% | −0.04375 |
| 17 | 624 | 21-40r1 1-10 | 1.62% | 0.015115 |
| 18 | 625 | 21-40r1 11-20 | 11.5% | 0.801114 |
| 19 | 626 | 21-40r1 1-5 | 3.48% | 0.163087 |
| 20 | 627 | 21-40r1 6-10 | 0.64% | −0.06285 |
| 21 | 628 | 21-40r1 11-15 | 9.81% | 0.666667 |
| 22 | 629 | 21-40r1 16-20 | 17.0% | 1.238663 |
| 23 | 632 | 21-40r2 11-20 | 0.84% | −0.04694 |
| 24 | 634 | 21-40r2 6-10 | 4.19% | 0.21957 |
| 25 | 398 | 2712 random3 | 1.61% | 0.01432 |
| 26 | 412 | 2712 random 17 | 0.86% | −0.04535 |
| 27 | 413 | SIINFEKL 121-140-6xHIS | 3.13% | 0.135243 |
| 28 | 602 | 16-20 | 2.33% | 0.071599 |
| 29 | 603 | 1-20r1 | 2.46% | 0.081941 |
| 30 | 604 | 1-20r1 1-10 | 4.04% | 0.207637 |
| 31 | ISG15 | | 11.0% | 0.761337 |

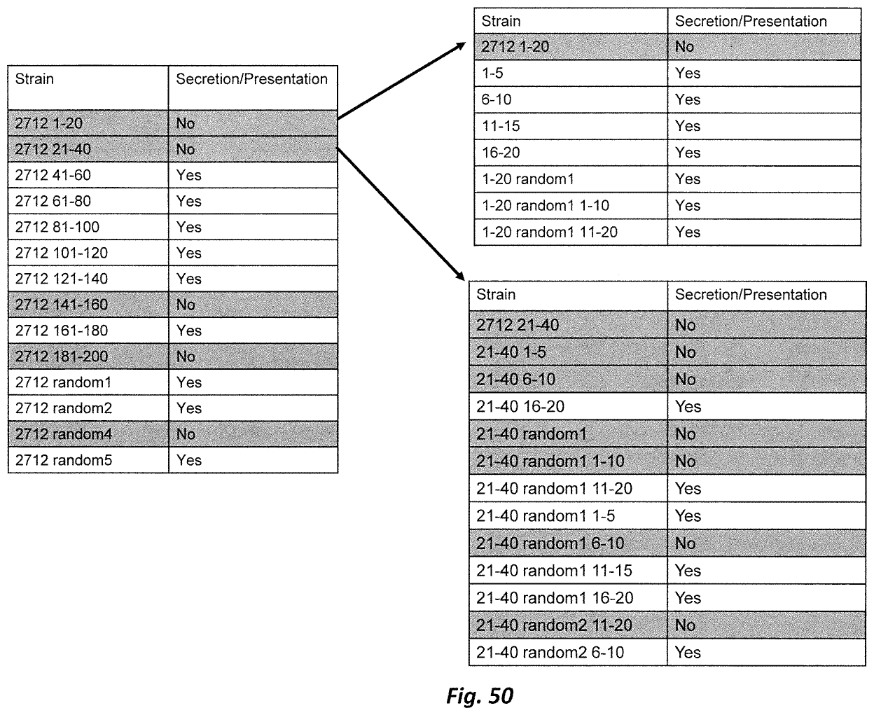

Detection of the C-terminal SIINFEKL tag with the 25D-APC conjugated antibody is shown in Table 17. As indicated in Table 17, the SVN positive control showed high levels of positive staining, whereas the no tag negative control showed a low level of staining. Similarly, samples 43-12, 15, 18, 19, 21, 22, 24, and 27-30 showed high levels of positive staining. This demonstrates confirmation that the neo-antigens express and secrete in antigen-presenting cells upon infection. FIG. 50 shows the effects of randomization of the order of neo-epitopes on presentation and secretion of the neo-epitopes. Ordering 1 thru 20 sequentially does not secrete. However, randomizing the entire order, or breaking down individual pieces, or randomizing those pieces results in successful secretion. Likewise, ordering 21-40 sequentially does not secrete. Individual regions of that 20′mer (1-5, 6-10) do not work, and other regions work (16-20). However, randomizing individual regions results in the successful secretion of each individual region.

### Example 32: Therapeutic Effects of Lm Neo-Antigen Constructs in B16F10 Murine Melanoma Model

After non-synonymous mutations are identified in cancer cells that are not present in corresponding healthy cells, major efforts are typically invested to determine the mutational functional impact, such as cancer driver versus passenger status, to form a basis for selecting therapeutic targets. However, little attention has been devoted to either define the immunogenicity of these mutations or characterize the immune responses they elicit. From the immunologic perspective, mutations may be particularly potent vaccination targets, as they can create neo-antigens that are not subject to central immune tolerance. When attention has been devoted to define the immunogenicity of these mutations or characterize the immune responses they elicit, efforts are typically directed to narrowing down the non-synonymous mutations to a single mutation to be included in a peptide for immunization. For example, in Castle et al., 962 non-synonymous point mutations were identified in B16F10 murine melanoma cells, with 563 of those mutations in expressed genes. Fifty of these mutations were selected based on selection criteria including low false discovery rate (FDR) confident value, location in an expressed gene, and predicted immunogenicity. Out of these 50, only 16 were found to elicit immune responses in immunized mice, and only 11 of the 16 induced an immune response preferentially recognizing the mutated epitope. Two of the mutations were then found to induce tumor growth inhibition. See, e.g., Castle et al. (2012) Cancer Res. 72(5):1081-1091, herein incorporated by reference in its entirety for all purposes. In the constructs described in the following experiments, however, our data suggest that Neo 20 and Neo 30 are better at controlling tumor growth. In our constructs, Neo-12 contains the 12 most immunogenic epitopes. Neo-12 contains both tumor controlling epitopes (Mut30 and Mut44, as disclosed above in Table 7). Neo 20 contains Mut30-Mut2-Mut3-Mut3-Mut4 . . . Mut19). Neo 30 contains Mut30-Mut2-Mut3 . . . Mut-29). Neo 20 and Neo 30 only contain one of the tumor controlling epitopes identified by Castle (Mut30), and then they contain both immunogenic and non-immunogenic epitopes. Despite not having multiple tumor controlling epitopes, and containing many non-tumor controlling and even non-immunogenic epitopes,

### Experiment 1

To determine therapeutic response generated by Lm neo-antigen constructs, a tumor regression study was designed to examine the therapeutic effects of such constructs on tumor growth in the B16F10 C57Bl/6 murine melanoma model. Specifically, Lm neo-antigen vectors were designed with 12 neo-antigens (Lm-Castle 12, containing Mut30, Mut5, Mut17, Mut20, Mut22, Mut24, Mut25, Mut44, Mut46, Mut48, and Mut50) or 20 neo-antigens (Lm-Castle 20, containing Mut30, Mut2, Mut3, Mut4, Mut5, Mut6, Mut7, Mut8, Mut9, Mut10, Mut11, Mut12, Mut13, Mut14, Mut15, Mut16, Mut17, Mut18, Mut19, and Mut20) identified by Castle et al. See, e.g., Castle et al. (2012) Cancer Res. 72(5):1081-1091, herein incorporated by reference in its entirety for all purposes.

Tumor Cell Line Expansion.

The B16F10 melanoma cell line was cultured in c-RPMI containing 10% FBS (50 mL) and 1× Glutamax (5 mL). The c-RPMI media includes the following components:

| | | |
|---|---|---|
| RPMI 1640 | 450 | mL |
| FCS | 50 | mL |
| HEPES | 5 | mL |
| NEAA | 5 | ml |
| L-Glutamine | 5 | mL |
| Na-Pyruvate | 5 | mL |
| Pen/step | 5 | mL |
| 2-ME (14.6M) | 129 | μL |

Tumor Inoculation.

On Day 0, B16F10 cells were trypsinized and washed twice with media. Cells were counted and re-suspended at a concentration of 1×10⁵ cells/200 uL of PBS for injection. B16F10 cells were then implanted subcutaneously in the right flank of each mouse. Mice were vaccinated on Day 3 of the study. Tumors were measured and recorded twice per week until reaching a size of 12 mm in diameter. Once tumors met sacrifice criteria, mice were euthanized, and tumors were excised and measured.

Immunotherapy Treatment.

On Day 3, immunotherapies and treatments began. Groups were treated with Lm (IP), and boosted twice. Details are listed in Table 18.

TABLE 18
Treatment Schedule.

| | B16F10 Tumor | Dose 1: | | |
|---|---|---|---|---|
| | Inoculation | Treatments at 1 | | |
| Groups | 1 × 10⁵ | week intervals | Dose 2: | Dose 3: |
| (10 mice/group) | cells/200 uL/mouse | 21 JAN. 2016 | 28 FEB. 2016 | 10 FEB. 2016 |
| 1-PBS ONLY | 18 JAN. 2016 | 200 uL/mouse | 200 uL/mouse | NA |
| (neg control) | | | | |
| 2-Poly (I:C) ONLY | 18 JAN. 2016 | (50 ug in 200 uL | (50 ug in 200 uL | NA |
| (50 ug in 200 uL PBS) | | PBS-SQ) | PBS- SQ) | |
| (neg control) | | | | |
| 3- LmddA-274 ONLY | 18 JAN. 2016 | 1 × 10⁸ IP | 1 × 10⁸ IP | NA |
| (neg control) | | | | |
| 4-Lm-Castle 12 | 18 JAN. 2016 | 1 × 10⁸ IP | 1 × 10⁸ IP | 1 × 10⁸ IP |
| (SEQ ID NO: 679) | | | | |
| 5-Lm Castle 20 | 18 JAN. 2016 | 1 × 10⁸ IP | 1 × 10⁸ IP | 1 × 10⁸ IP |
| (SEQ ID NO: 680) | | | | |

Immunotherapy Treatment Preparation.

1. PBS ONLY—200 uL/mouse IP.

2. LmddA-274 (Titer: 1.5×10⁹ CFU/mL)
a. Thaw 1 vial from −80° C. in 37° C. water bath.
b. Spin at 14,000 rpm for 2 min and discard supernatant.
c. Wash 2 times with 1 mL PBS and discard PBS.
d. Re-suspend in PBS to a final concentration of 5×10⁸ CFU/mL.

3. Lm-Castle 12 (Titer: 1.59×10⁹ CFU/mL and Lm-Castle 20 (Titer: 1.6×10⁹ CFU/mL)
a. Thaw 1 vial from −80° C. in 37° C. water bath.
b. Spin at 14,000 rpm for 2 min and discard supernatant.
c. Wash 2 times with 1 mL PBS and discard PBS.
d. Re-suspend in PBS to a final concentration of 5×10⁸ CFU/mL.

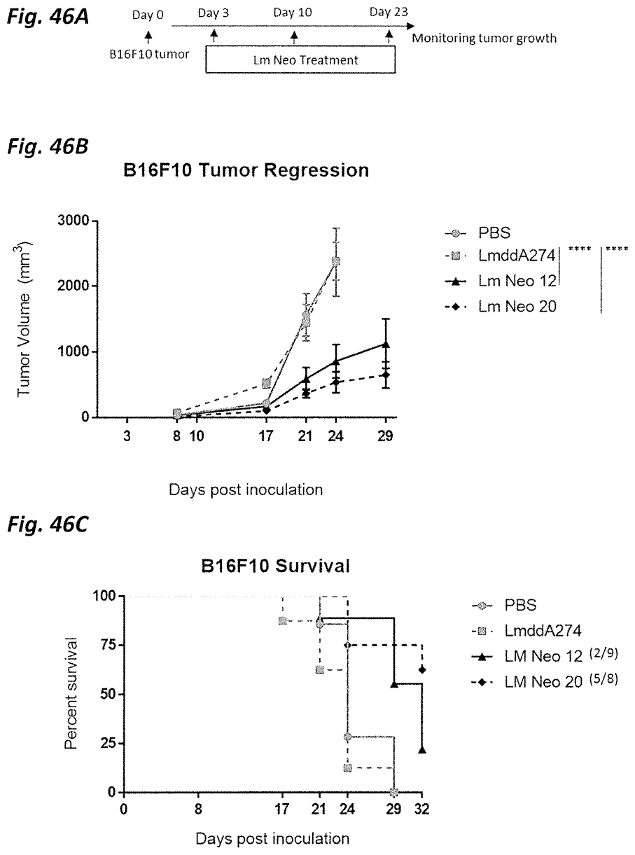

As shown in FIG. 46B, growth of tumors was inhibited by Lm-Neo 12 and Lm-Neo 20 as compared with the control groups (PBS and LmddA274). LmddA274 is the Listeria control, and is an empty vector. It includes the truncated LLO (tLLO), however no neo-epitopes are attached. In addition, Lm-Neo 20, which contained 20 neo-antigens, inhibited tumor growth to a greater extent than Lm-Neo 12, which contained 12 neo-antigens. Likewise, Lm-Neo 20 and Lm-Neo 12 each result in increased survival time when compared with the control groups, with Lm-Neo 20 providing the greatest protective effect (FIG. 46C). These data show that vaccination with Lm carrying neo-epitopes is able to confer antitumoral effects, and increasing the number of neo-epitopes increases the antitumoral effects.

### Experiment 2

To further compare therapeutic responses generated by different Lm neo-antigen constructs, a tumor regression study was designed to examine the therapeutic effects of such constructs on tumor growth in the B16F10 C57Bl/6 murine melanoma model. Specifically, Lm neo-antigen vectors were designed with 12 neo-antigens (Lm-Castle 12), 20 neo-antigens (Lm-Castle 20), or 39 neo-antigens (Lm-Castle 39; no linker, no 20-29 (Lm-Castle 30)) identified by Castle et al. See, e.g., Castle et al. (2012) Cancer Res. 72(5):1081-1091, herein incorporated by reference in its entirety for all purposes.

Tumor Cell Line Expansion.

The B16F10 melanoma cell line was cultured in c-RPMI containing 10% FBS (50 mL) and 1× Glutamax (5 mL).

Tumor Inoculation.

On Day 0, B16F10 cells were trypsinized and washed twice with media. Cells were counted and re-suspended at a concentration of 1×10⁵ cells/200 uL of PBS for injection. B16F10 cells were then implanted subcutaneously in the right flank of each mouse. Mice were vaccinated on Day 4 of the study. Tumors were measured and recorded twice per week until reaching a size of 1500 mm³ in volume. Once tumors met sacrifice criteria, mice were euthanized, and tumors were excised and measured.

Immunotherapy Treatment.

On Day 4, immunotherapies and treatments began. Animals were treated once every 7 days until the end of the study. Groups were treated with either PBS, LmddA274, Lm-Castle 12, Lm-Castle 20, Lm-Castle 39 no linker no 20-29, detailed in Table 19.

TABLE 19
Treatment Schedule.

| | B16F10 | | | | | |
|---|---|---|---|---|---|---|
| | Tumor | | | | | |
| | Inoculation | | | | | |
| Groups | 1 × 10⁵ | | | | | |
| (10 = N/ | cells/200 uL/ | Dose 1: | Dose 2: | Dose 3: | Dose 4: | Dose 5: |
| group) | mouse | 01MAR16 | 08MAR16 | 15MAR16 | 22MAR16 | 29MAR16 |
| 1-PBS | 26FEB16 | 200 uL/ | 200 uL/ | 200 uL/ | 200 uL/ | 200 uL/ |
| ONLY | | Mouse | Mouse | Mouse | Mouse | Mouse |
| (neg | | IP | IP | IP | IP | IP |
| control) | | | | | | |
| 2-LmddA- | 26FEB16 | 1 × 10⁸ IP | 1 × 10⁸ IP | 1 × 10⁸ IP | 1 × 10⁸ IP | 1 × 10⁸ IP |
| 274 ONLY | | | | | | |
| (neg | | | | | | |
| control) | | | | | | |
| 3-Lm | 26FEB16 | 1 × 10⁸ IP | 1 × 10⁸ IP | 1 × 10⁸ IP | 1 × 10⁸ IP | 1 × 10⁸ IP |
| Castle 12 | | | | | | |
| (SEQ ID | | | | | | |
| NO: 679) | | | | | | |
| 4-Lm | 26FEB16 | 1 × 10⁸ IP | 1 × 10⁸ IP | 1 × 10⁸ IP | 1 × 10⁸ IP | 1 × 10⁸ IP |
| Castle 20 | | | | | | |
| (SEQ ID | | | | | | |
| NO: 680) | | | | | | |
| 5-Lm | 26FEB16 | 1 × 10⁸ IP | 1 × 10⁸ IP | 1 × 10⁸ IP | 1 × 10⁸ IP | 1 × 10⁸ IP |
| Castle 39 | | | | | | |
| (no link no | | | | | | |
| 20-29) | | | | | | |
| (also | | | | | | |
| called Lm | | | | | | |
| Castle 30) | | | | | | |
| (SEQ ID | | | | | | |
| NO: 681) | | | | | | |

Immunotherapy Treatment Preparation.
1. PBS ONLY—200 uL/mouse IP.
2. LmddA-274 (Titer: 1.7×10⁹ CFU/mL)
   a. Thaw 1 vial from −80° C. in 37° C. water bath.
   b. Spin at 14,000 rpm for 2 min and discard supernatant.
   c. Wash 2 times with 1 mL PBS and discard PBS.
   d. Re-suspend in PBS to a final concentration of 5×10⁸ CFU/mL.
3. Lm-Castle 12 (Titer: 1.59×10⁹ CFU/mL and Lm-Castle 20 (Titer: 1.6×10⁹ CFU/mL) and Lm-Castle 39) Titer: 1×10⁹ CFU/mL)
   a. Thaw 1 vial from −80° C. in 37° C. water bath.
   b. Spin at 14,000 rpm for 2 min and discard supernatant.
   c. Wash 2 times with 1 mL PBS and discard PBS.
   d. Re-suspend in PBS to a final concentration of 5×10⁸ CFU/mL.

Harvesting Details.

The spleen from each mouse was collected in an individual tube containing 5 mL of c-RPMI medium. Detailed steps are described below. All tumors were excised and measured at termination of the study.
1. Harvest spleens using sterile forceps and scissors.
2. Mash each spleen in wash medium (RPMI only) using two glass slides or the back of plunger from a 3 mL syringe.
3. Transfer cells in the medium to a 15 mL tube.
4. Pellet cells at 1,000 RPM for 5 min at room temperature.
5. Discard supernatant, re-suspend cells in the remaining wash buffer gently, and add 2 mL RBC lysis buffer per spleen to the cell pellet. Mix cells gently with lysis buffer by tapping the tube and wait for 1 min.
6 Immediately add 10 mL of c-RPMI medium to the cell suspension to deactivate the lysis buffer.
7. Spin cells at 1,000 for 5 min at room temperature.
8. Pass the cells through a cell strainer and wash them one more time with 10 mL c-RPMI.
9. Count cells using hemocytometer/moxi flow and check the viability by Trypan blue staining. Each spleen should yield ˜1-2×10⁸ cells.
10. Divide the cells for staining
11. Follow immudex dextramer staining protocol: with the one exception of adding the cell surface antibodies (CD8, CD62L) in 2.4G2 instead of staining buffer (www.immudex.com/media/12135/tf1003.03_general_staining_procedure_mhc_dextramer.pdf).

CD8+ T Cell Response.

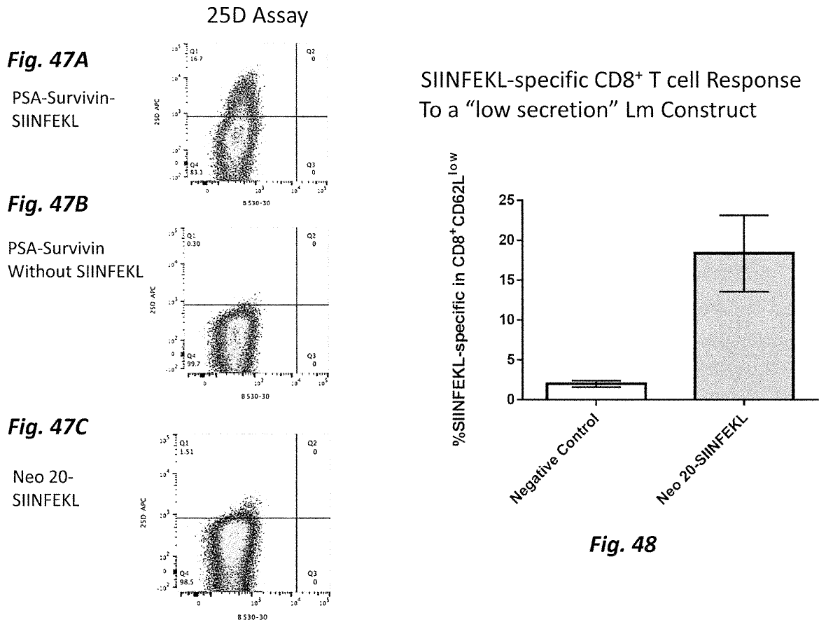

25D assays were done as explained above to measure expression and secretion of the Lm-Neo 20 construct in antigen presenting cells. FIG. 47A is a positive control (PSA-Survivin-SIINFEKL), FIG. 47B is a negative control (PSA-Survivin without SIINFEKL), and FIG. 47C is the Lm-Neo 20 (with SIINFEKL tag at C-terminus). As indicated in FIG. 47, the Lm-Neo 20 expresses and is secreted, but only at low levels compared to the positive control. However, despite these low secretion levels, a specific CD8+ T cell response to SIINFEKL was observed. FIG. 48 shows the SIINFEKL-specific CD8+ T cell response to the “low secretion” Lm-Neo 20 construct. As shown in FIG. 48, approximately 20% of the CD8+ T cells are specific for antigens in the Lm Neo 20 construct.

Antitumor Effects.

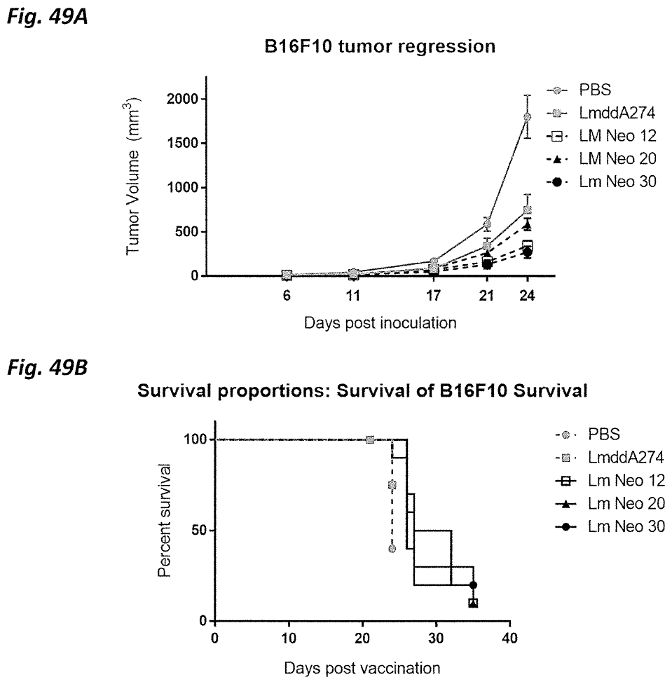

As shown in FIG. 49A, growth of tumors was inhibited by Lm-Neo 12, Lm-Neo 20, and Lm-Neo 30 as compared with the control groups (PBS and Lmdd A274). In addition, Lm-Neo 30, which contained 30 neo-antigens, inhibited tumor growth to a greater extent than Lm-Neo 20, which contained 20 neo-antigens, which inhibited tumor growth to a greater extent than Lm-Neo 12, which contained 12 neo-antigens. Likewise, Lm-Neo 30, Lm-Neo 20, and Lm-Neo 12 each result in increased survival time when compared with the control groups, with Lm-Neo 30 providing the greatest protective effect and Lm-Neo 20 providing the next greatest protective effect (FIG. 46C). These data show that vaccination with Lm carrying neo-epitopes is able to confer antitumoral effects, and increasing the number of neo-epitopes increases the antitumoral effects.

### Example 33: Neo-Epitope-Specific Immunity in Mice Immunized with Lm Neo-Antigen Constructs

An experiment was designed to evaluate the generation of neo-epitope and signal peptide-specific responses in C57BL/6 mice after immunization with r M2, 1-20, 81-100, 101-120, 121-140, Lm 2712#1 & Lm 2712#3 neo-epitope constructs. The neo-epitope and signal peptide-specific immune response will be detected by pentamer staining using the known T cell H-2 D^{b} PSA65-73 (HCIRNKSVI), VvB8R(TSYKPBSV), IAV PA(SSLENFRAYV) as well as neo-epitope peptide-specific responses as evaluated by intracellular cytokine staining for IFN-γ. The details of immunization schedule and strains are given in Table 20.

TABLE 20
Immunization Schedule.

| | Titer- | | | | | Spleen |
|---|---|---|---|---|---|---|
| Immunotherapy/Group | CFU/mL | Mice/Group | Dose 1 | Dose 2 | Dose 3 | harvest |
| 1) r M 2 (VV B8R) | 1.04 × 10⁹ | 5 | 22MAR16 | 05APR16 | 19APR16 | 26APR16 |
| 2) 1-20 | 1.16 × 10⁹ | 5 | 22MAR16 | 05APR16 | 19APR16 | 26APR16 |
| 3) 81-100 | 1.12 × 10⁹ | 5 | 22MAR16 | 05APR16 | 19APR16 | 26APR16 |
| 4) 101-120 | 1.09 × 10⁹ | 5 | 22MAR16 | 05APR16 | 19APR16 | 26APR16 |
| 5) 121-140 | 0.96 × 10⁹ | 5 | 22MAR16 | 05APR16 | 19APR16 | 26APR16 |
| 6) 2712#1 (PSA) | 0.95 × 10⁹ | 5 | 22MAR16 | 05APR16 | 19APR16 | 26APR16 |
| 7) 2712#3 (IAV PA | 1 × 10⁹ | 5 | 22MAR16 | 05APR16 | 19APR16 | 26APR16 |

Each of these constructs target mutations from the same 2712 Lung sample used in all of the lung construct experiments. rM2 is a random order of 20 21′mers from the 200 non-synonomous mutation pool. 1-20 includes the first 20 non-synonomous mutations, in that order. The same applies for 81-100, 101-120, and 121-140. 2712#1 includes 50 21-mers (1-50). 2712 #3 includes 50 21-mers (101-150).

Immunotherapy Preparation.
1. Thaw 1 vial from −80° C. in 37° C. water bath.
2. Spin at 14,000 rpm for 2 min and discard supernatant.
3. Wash 2 times with 1 mL PBS and discard PBS.
4. Re-suspend in PBS to appropriate final concentration.

Preparing Isolated Splenocytes.
1. Harvest spleens using sterile forceps and scissors.
2. Mash each spleen in wash medium (RPMI only) using two glass slides or the back of plunger from a 3 mL syringe.
3. Transfer cells in the medium to a 15 mL tube.
4. Pellet cells at 1,000 RPM for 5 min at room temperature.
5. Discard supernatant, re-suspend cells in the remaining wash buffer gently, and add 2 mL RBC lysis buffer per spleen to the cell pellet. Mix cells gently with lysis buffer by tapping the tube and wait for 1 min.
6 Immediately add 10 mL of c-RPMI medium to the cell suspension to deactivate the lysis buffer.
7. Spin cells at 1,000 for 5 min at room temperature.
8. Pass the cells through a cell strainer and wash them one more time with 10 mL c-RPMI.
9. Count cells using hemocytometer/moxi flow and check the viability by Trypan blue staining. Each spleen should yield ˜1-2×10⁸ cells.
10. Divide the cells for pentamer staining and ELISpot.

ELISPOT for IFN Gamma

Day 1. Tubes to Prepare:

PMA (dilute 1:1000 in complete medium). Cells were prepared as mentioned below (5×10⁶/ml) for each mouse).

| | | | |
|---|---|---|---|
| 1) | Prepare complete medium (with BME) | 100 | mL |
| 2) | Grp 1/mouse 1 cells (5 × 10⁶/mL of complete medium with BME) | 2 | mL |
| 3) | Grp 2/mouse 1 cells (5 × 10⁶/mL of complete medium with BME) | 2 | mL |
| 4) | Grp 3/mouse 1 cells (5 × 10⁶/mL of complete medium with BME) | 2 | mL |
| 5) | Grp 1/mouse 1 cells (5 × 10⁴/mL of complete medium with BME) | 2 | mL |
| 6) | Grp 2/mouse 1 cells (5 × 10⁴/mL of complete medium with BME) | 2 | mL |
| 7) | Grp 3/mouse 1 cells (5 × 10⁴/mL of complete medium with BME) | 2 | mL |
| 8) | No peptide (Medium from tube 1) | 3 | mL |
| 9) | E7 peptide (add 2 ul of 1 mM peptide/mL of medium from tube 1)− | 16 | mL |
| 10) | PMA (add 10 ul of 1 ug/mL stock/mL of medium from tube 1) + Ionomycin (add 1 | 5 | mL |
| | μL of 1 mg/mL stock/mL of medium from tube 1) | | |

Stimulation with Peptide:
a) Wash plate 4 times with sterile PBS (200 uL/well).
b) Add 200 uL/well of complete medium. Incubate for at least 30 min at RT.
c) Remove the medium and add the cell suspension (100 uL/well)+stimulants (100 uL/well) as planned in the table.
d) Wrap the plate in aluminum foil and incubate the plate at 37° C., 5% CO₂ for 24 h.

Day 2. Detection of Spots.
a) Remove cells by emptying the plate and wash 5 times with PBS (200 uL/well).
b) Add 100 uL/well of diluted R4-6A2-biotin and incubate for 2 h at room temperature.
c) Wash 5 times with PBS (200 uL/well).
d) Add 100 uL/well of diluted Streptavidin-ALP and incubate for 1 h at room temperature.
e) Wash 5 times with PBS (200 uL/well).
f) Add 100 uL/well of filtered (0.45 μm filter) ready to use substrate solution (BCIP/NBT) and develop until distinct spots emerge.
g) Stop color development by washing extensively in tap water.
h) Leave the plate to dry and count spots the next day.

| Antibody | Dilutions (uL) |
|---|---|
| R4-6A2 | 40 uL + 39960 PBS with 0.5% FCS |
| Streptavidin-ALP | 40 uL + 39960 PBS with 0.5% FCS |

Results.

Table 21 details whether we were able to detect secretion of the 21′mers via 25D assay.

TABLE 21
Lung immunogenicity summary

| Strain | Secretion/presentation by 25D Assay |
|---|---|
| 1) rM 2 (VV B8R) | No |
| 2) 1-20 | No |
| 3) 81-100 | Minimal but positive |
| 4) 101-120 | Yes |
| 5) 121-140 | Yes |
| 6) 2712 #1 | No |
| 7) 2712 #3 | No |

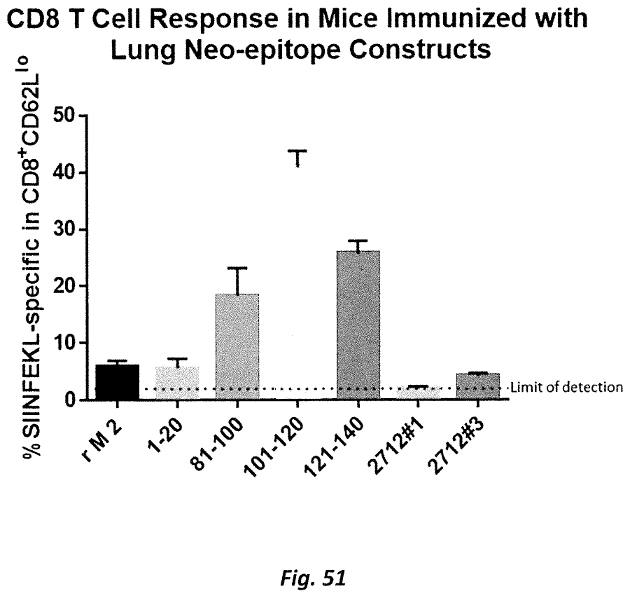

FIG. 51. summarizes the SIINFEKL-specific CD8 T cell response in mice immunized with the various constructs. An immune response against SIINFEKL (e.g. surrogate tag for the neo-epitope 21-mer amino acid chain being secreted into the host and the host generating an immune response against the 21-mer amino acid chain), was detected in all constructs, except the 2712 #1 50-21-mer construct.

Of importance, 3 constructs (r M 2, 1-20 & 2712 #3) that did not screen as a positive screener via 25D assay did in fact generate an in vivo immune response (although the response is less pronounced than constructs that screened positive by 25D). Additionally, an immune response to constructs up to 50 21-mers was able to be generated.

### Example 34: Testing of 27-mers

A 25D assay was performed, as described above, using Neo-eptiope constructs comprised of 27 amino acid “27-mers.” The results are shown below in Table 22, and show that the constructs may be composed of oligomers other than 21-mers.

TABLE 22
Detection of 25D-APC (Assay using 27-mers)

| | | | Sequence: Neo-eptiope constructs |
|---|---|---|---|
| | % 25D | | comprised of 27 amino acid: |
| Name | Positive | Pass | “27′mers.” |
| Svn | 14.6% | YES | Same PSA-Survivin-SIINFEKL-tag |
| | | | sequence in all other 25D assays |
| No Tag | 2.66% | NO | Same PSA-Survivin-no tag sequence |
| | | | in all other 25D assays |
| B16_6 | 16.6% | YES | SEQ ID NO: 682 |
| B16_8 | 17.2% | YES | SEQ ID NO: 683 |
| B16_1-13 | 9.61% | YES | SEQ ID NO: 684 |
| B16_14-27 | 2.71% | NO | SEQ ID NO: 685 |
| B16_M30 | 20.4% | YES | SEQ ID NO: 686 |
| B16_M20 | 17.4% | YES | SEQ ID NO: 687 |
| B16_M50 | 2.23% | YES | SEQ ID NO: 688 |

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A process for creating a personalized immunotherapy for a subject having a disease or condition, the process comprising the steps of:
(a) comparing one or more open reading frames (ORFs) in nucleic acid sequences extracted from a disease-bearing biological sample from the subject with one or more ORFs in nucleic acid sequences extracted from a healthy biological sample, wherein the comparing identifies one or more nucleic acid sequences encoding one or more peptides comprising one or more neo-epitopes encoded within the one or more ORFs from the disease-bearing sample;
(b) transforming an attenuated Listeria strain with a vector comprising a nucleic acid sequence encoding the one or more peptides comprising the one or more neo-epitopes identified in step (a); and
(c) alternatively (i) storing the attenuated recombinant Listeria strain for administering to the subject at a pre-determined period, or (ii) administering a composition comprising the attenuated recombinant Listeria strain to the subject, wherein the administering results in the generation of a personalized T-cell immune response against the disease or condition.

2. The process of claim 1, further comprising:
(d) obtaining a second biological sample from the subject comprising a T-cell clone or T-infiltrating cell from the T-cell immune response in step (c) and characterizing specific peptides comprising one or more immunogenic neo-epitopes bound by MHC Class I or MHC Class II molecules on the T cells;
(e) screening for and selecting a nucleic acid construct encoding the one or more peptides comprising the one or more immunogenic neo-epitopes identified in step (d);
(f) transforming a second attenuated recombinant Listeria strain with a vector comprising a nucleic acid sequence encoding the one or more peptides comprising the one or more immunogenic neo-epitopes; and
(g) alternatively (i) storing the second attenuated recombinant Listeria for administering to the subject at a pre-determined period, or (ii) administering a second composition comprising the second attenuated recombinant Listeria strain to the subject.

3. The process of any preceding claim, wherein each of the one or more peptides comprising the one or more neo-epitopes is about 5-50 amino acids in length.

4. The process of claim 3, wherein each of the one or more peptides comprising the one or more neo-epitopes is about 8-27 amino acids in length.

5. The process of any preceding claim, wherein the one or more neo-epitopes comprise 5-100 neo-epitopes.

6. The process of claim 5, wherein the one or more neo-epitopes comprise 15-35 neo-epitopes, 8-11 neo-epitopes or 11-16 neo-epitopes.

7. The process of any preceding claim, wherein the one or more neo-epitopes comprise a plurality of neo-epitopes, wherein step (b) further comprises one or more iterations of randomizing the order of the one or more peptides comprising the plurality of neo-epitopes within the nucleic acid sequence of step (b).

8. The process of any preceding claim, wherein the process is repeated to create a plurality of attenuated recombinant Listeria strains, each comprising a different set of one or more neo-epitopes.

9. The process of claim 8, wherein the plurality of attenuated recombinant Listeria strains comprises 5-10, 10-15, 15-20, 10-20, 20-30, 30-40, or 40-50 attenuated recombinant Listeria strains.

10. The process of claim 8 or 9, wherein the combination of the plurality of attenuated recombinant Listeria strains comprises about 5-10, 10-15, 15-20, 10-20, 20-30, 30-40, 40-50, 50-60, 60-70, 70-80, 80-90, 90-100, or 100-200 neo-epitopes.

11. The process of any preceding claim, wherein the comparing in step (a) comprises use of a screening assay or screening tool and associated digital software for comparing the one or more ORFs in the nucleic acid sequences extracted from the disease-bearing biological sample with the one or more ORFs in the nucleic acid sequences extracted from the healthy biological sample,
wherein the associated digital software comprises access to a sequence database that allows screening of mutations within the ORFs in the nucleic acid sequences extracted from the disease-bearing biological sample for identification of immunogenic potential of the neo-epitopes.

12. The process of any preceding claim, wherein the process of obtaining a second biological sample from the subject in step (d) comprises obtaining a biological sample comprising T-cell clones or T-infiltrating cells that expand following administration of the composition comprising the attenuated recombinant Listeria strain.

13. The process of claim 12, wherein the disease-bearing biological sample is tissue, cells, blood, or sera.

14. The process of any preceding claim, wherein the process of characterizing in step (d) comprises the steps of:
(i) identifying, isolating, and expanding T cell clones or T-infiltrating cells that respond against the disease; and
(ii) screening for and identifying one or more peptides comprising one or more immunogenic neo-epitopes loaded on specific MHC Class I or MHC Class II molecules to which a T-cell receptor on the T cells binds.

15. The process of claim 14, wherein the screening for and identifying in step (ii) comprises T-cell receptor sequencing, multiplex based flow cytometry, or high-performance liquid chromatography.

16. The process of claim 15, wherein the sequencing comprises the use of associated digital software and database.

17. The process of any preceding claim, wherein the disease or condition is an infectious disease, a tumor, or a cancer.

18. The process of any preceding claim, wherein the infectious disease comprises a viral or bacterial infection.

19. The process of any preceding claim, wherein the healthy biological sample is obtained from the subject having the disease or condition.

20. The process of any preceding claim, wherein the nucleic acid sequences extracted from the disease-bearing biological sample and the nucleic acid sequences extracted from the healthy biological sample are determined using exome sequencing or transcriptome sequencing.

21. The process of any preceding claim, wherein the one or more neo-epitopes comprise linear neo-epitopes.

22. The process of any preceding claim, wherein the one or more neo-epitopes comprise a solvent-exposed epitope.

23. The process of any preceding claim, wherein the attenuated recombinant Listeria secretes the one or more peptides comprising the one or more neo-epitopes.

24. The process of any preceding claim, wherein the one or more neo-epitopes comprise a T-cell epitope.

25. The process of any preceding claim, wherein the transforming in step (b) is accomplished using a plasmid or phage vector.

26. The process of any preceding claim, wherein the one or more peptides comprising the one or more neo-epitopes are each fused to an immunogenic polypeptide or fragment thereof.

27. The process of any preceding claim, wherein the transforming in step (b) is accomplished using a plasmid vector comprising a minigene nucleic acid construct, the construct comprising one or more open reading frames encoding a chimeric protein, wherein the chimeric protein comprises:
a. a bacterial secretion signal sequence;
b. a ubiquitin (Ub) protein; and
c. the one or more peptides comprising the one or more neo-epitopes,
wherein the bacterial secretion signal sequence, the ubiquitin protein, and the one or more peptides are operatively linked or arranged in tandem from the amino-terminus to the carboxy-terminus.

28. The process of any preceding claim, wherein step (b) further comprises culturing and characterizing the attenuated recombinant Listeria strain to confirm expression and secretion of the one or more peptides.

29. The process of any preceding claim 25, wherein the plasmid is an integrative plasmid.

30. The process of claim 29, wherein the plasmid is an extrachromosomal multicopy plasmid.

31. The process of claim 29 or 30, wherein the plasmid is stably maintained in the Listeria strain in the absence of antibiotic selection.

32. The process of claim 26, wherein the immunogenic polypeptide is a mutated Listeriolysin O (LLO) protein, a truncated LLO (tLLO) protein, a truncated ActA protein, or a PEST amino acid sequence.

33. The process of claim 32, wherein the tLLO protein is set forth in SEQ ID NO: 3.

34. The process of claim 32, wherein the actA is set forth in SEQ ID NO: 12-13 and 15-18.

35. The process of claim 32, wherein the PEST amino acid sequence is selected from the sequences set forth in SEQ ID NOs: 5-10.

36. The process of claim 32, wherein the mutated LLO comprises a mutation in a cholesterol-binding domain (CBD).

37. The process of claim 36, wherein the mutation comprises a substitution of residue C484, W491, or W492 of SEQ ID NO: 2, or any combination thereof.

38. The process of claim 36, wherein the mutation comprises a substitution of 1-11 amino acid within the CBD set forth in SEQ ID NO: 68 with a 1-50 amino acid non-LLO peptide, wherein the non-LLO peptide comprises a peptide comprising a neo-epitope.

39. The process of claim 36, wherein the mutation comprises a deletion of a 1-11 amino acid within the CBD as set forth in SEQ ID NO: 68.

40. The process of any preceding claim, wherein the one or more peptides comprise a heterologous antigen or a self-antigen associated with the disease.

41. The process of any preceding claim, wherein the heterologous antigen or the self-antigen is a tumor-associated antigen or a fragment thereof.

42. The process of any preceding claim, wherein the one or more neo-epitopes comprise a cancer-specific or tumor-specific epitope.

43. The process of any preceding claim, wherein the tumor-associated antigen or fragment thereof comprises a Human Papilloma Virus (HPV)-16-E6, HPV-16-E7, HPV-18-E6, HPV-18-E7, a Her/2-neu antigen, a chimeric Her2 antigen, a Prostate Specific Antigen (PSA), bivalent PSA, ERG, Androgen receptor (AR), PAK6, Prostate Stem Cell Antigen (PSCA), NY-ESO-1, a Stratum Corneum Chymotryptic Enzyme (SCCE) antigen, Wilms tumor antigen 1 (WT-1), HIV-1 Gag, human telomerase reverse transcriptase (hTERT), Proteinase 3, Tyrosinase Related Protein 2 (TRP2), High Molecular Weight Melanoma Associated Antigen (HMW-MAA), synovial sarcoma, X (SSX)-2, carcinoembryonic antigen (CEA), Melanoma-Associated Antigen E (MAGE-A, MAGE 1, MAGE2, MAGE3, MAGE4), interleukin-13 Receptor alpha (IL13-R alpha), Carbonic anhydrase IX (CAIX), survivin, GP100, an angiogenic antigen, a ras protein, a p53 protein, a p97 melanoma antigen, KLH antigen, carcinoembryonic antigen (CEA), gp100, MART1 antigen, TRP-2, HSP-70, beta-HCG, or Testisin.

44. The process of any preceding claim, wherein the tumor or cancer comprises a breast cancer or tumor, a cervical cancer or tumor, a Her2-expressing cancer or tumor, a melanoma, a pancreatic cancer or tumor, an ovarian cancer or tumor, a gastric cancer or tumor, a carcinomatous lesion of the pancreas, a pulmonary adenocarcinoma, a glioblastoma multiforme, a colorectal adenocarcinoma, a pulmonary squamous adenocarcinoma, a gastric adenocarcinoma, an ovarian surface epithelial neoplasm, an oral squamous cell carcinoma, non-small-cell lung carcinoma, an endometrial carcinoma, a bladder cancer or tumor, a head and neck cancer or tumor, a prostate carcinoma, a renal cancer or tumor, a bone cancer or tumor, a blood cancer, or a brain cancer or tumor.

45. The process of any preceding claim, wherein the one or more neo-epitopes comprise an infectious-disease-associated epitope.

46. The process of any preceding claim, wherein the infectious disease is an infectious viral disease.

47. The process of any preceding claim, wherein the infectious disease is an infectious bacterial disease.

48. The process of claim 47, wherein the infectious disease is caused by one of the following pathogens: Leishmania, Entamoeba histolytica (which causes amebiasis), Trichuris, BCG/Tuberculosis, Malaria, Plasmodium falciparum, Plasmodium malariae, Plasmodium vivax, Rotavirus, Cholera, Diptheria-Tetanus, Pertussis, Haemophilus influenzae, Hepatitis B, Human papilloma virus, Influenza seasonal), Influenza A (H1N1) Pandemic, Measles and Rubella, Mumps, Meningococcus A+C, Oral Polio Immunotherapies, mono, bi and trivalent, Pneumococcal, Rabies, Tetanus Toxoid, Yellow Fever, Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers, Arenaviruses (LCM, Junin virus, Machupo virus, Guanarito virus, Lassa Fever), Bunyaviruses (Hantaviruses, Rift Valley Fever), Flaviruses (Dengue), Filoviruses (Ebola, Marburg), Burkholderia pseudomallei, Coxiella burnetii (Q fever), Brucella species (brucellosis), Burkholderia mallei (glanders), Chlamydia psittaci (Psittacosis), Ricin toxin (from Ricinus communis), Epsilon toxin of Clostridium perfringens, Staphylococcus enterotoxin B, Typhus fever (Rickettsia prowazekii), other Rickettsias, Food- and Waterborne Pathogens, Bacteria (Diarrheagenic E. coli, Pathogenic Vibrios, Shigella species, Salmonella BCG/, Campylobacter jejuni, Yersinia enterocolitica), Viruses (Caliciviruses, Hepatitis A, West Nile Virus, LaCrosse, Calif. encephalitis, VEE, EEE, WEE, Japanese Encephalitis Virus, Kyasanur Forest Virus, Nipah virus, hantaviruses, Tickborne hemorrhagic fever viruses, Chikungunya virus, Crimean-Congo Hemorrhagic fever virus, Tickborne encephalitis viruses, Hepatitis B virus, Hepatitis C virus, Herpes Simplex virus (HSV), Human immunodeficiency virus (HIV), Human papillomavirus (HPV)), Protozoa (Cryptosporidium parvum, Cyclospora cayatanensis, Giardia lamblia, Entamoeba histolytica, Toxoplasma), Fungi (Microsporidia), Yellow fever, Tuberculosis, including drug-resistant TB, Rabies, Prions, Severe acute respiratory syndrome associated coronavirus (SARS-CoV), Coccidioides posadasii, Coccidioides immitis, Bacterial vaginosis, Chlamydia trachomatis, Cytomegalovirus, Granuloma inguinale, Hemophilus ducreyi, Neisseria gonorrhea, Treponema pallidum, Streptococcus mutans, or Trichomonas vaginalis.

49. The process of any preceding claim, wherein the attenuated Listeria comprises a mutation in one or more endogenous genes.

50. The process of any preceding claim, wherein the mutation is selected from an actA gene mutation, a prfA mutation, an actA and inlB double mutation, a dal/dal gene double mutation, a dal/dat/actA gene triple mutation, or a combination thereof.

51. The process of any preceding claim, wherein the mutation comprises an inactivation, truncation, deletion, replacement, or disruption of the one or more endogenous genes.

52. The process of any preceding claim, wherein the vector further comprises an open reading frame encoding a metabolic enzyme.

53. The process of claim 52, wherein the metabolic enzyme is an alanine racemase enzyme or a D-amino acid transferase enzyme.

54. The process of any preceding claim, wherein the Listeria is Listeria monocytogenes.

55. The process of any preceding claim, wherein step (c)(ii) further comprises administering an adjuvant to the subject.

56. The process of claim 55, wherein the adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

57. The process of any preceding claim, wherein step (c)(ii) further comprises administering an immune checkpoint inhibitor antagonist.

58. The process of claim 57, wherein the immune checkpoint inhibitor is an anti-PD-L1/PD-L2 antibody or fragment thereof, an anti-PD-1 antibody or fragment thereof, an anti-CTLA-4 antibody or fragment thereof, or an anti-B7-H4 antibody or fragment thereof.

60. The process of any preceding claim, wherein the administering in step (c)(ii) generates a personalized enhanced anti-disease or anti-condition immune response in the subject.

61. The process of claim 60, wherein the immune response comprises an anti-cancer or anti-tumor response.

62. The process of claim 60, wherein the immune response comprises an anti-infectious disease response.

63. The process of claim 62, wherein the infectious disease comprises a viral infection.

64. The process of claim 62, wherein the infectious disease comprises a bacterial infection.

65. The process of any preceding claim, wherein the process allows personalized treatment or prevention of the disease or condition in the subject.

66. The process of any preceding claim, wherein the personalized immunotherapy increases survival time in the subject having the disease or condition.

67. A recombinant attenuated Listeria strain produced by the process of any one of claims 1-66.

68. A process for creating a personalized immunotherapy for a subject having a disease or condition, the process comprising the steps of:
(a) comparing one or more open reading frames (ORFs) in nucleic acid sequences extracted from a disease-bearing biological sample with one or more ORFs in nucleic acid sequences extracted from a healthy biological sample, wherein the comparing identifies one or more nucleic acid sequences encoding one or more peptides comprising one or more neo-epitopes encoded within the one or more ORFs from the disease-bearing sample;
(b) transforming a vector with a nucleic acid sequence encoding the one or more peptides comprising the one or more neo-epitopes identified in step (a), or generating a DNA immunotherapy vector or a peptide immunotherapy vector using the nucleic acid sequence encoding the one or more peptides comprising the one or more neo-epitopes identified in step (a); and
(c) alternatively (i) storing the vector or the DNA immunotherapy or the peptide immunotherapy for administering to the subject at a pre-determined period, or (ii) administering a composition comprising the vector, the DNA immunotherapy, or the peptide immunotherapy to the subject, and wherein the administering results in the generation of a personalized T-cell immune response against the disease or condition.

69. The process of claim 68, further comprising:
(d) obtaining a second biological sample from the subject comprising a T-cell clone or T-infiltrating cell from the T-cell immune response in step (c) and characterizing specific peptides comprising one or more immunogenic neo-epitopes bound by MHC Class I or MHC Class II molecules on the T cells;
(e) screening for and selecting a nucleic acid construct encoding the one or more peptides comprising the one or more immunogenic neo-epitopes identified in step (d);
(f) transforming a second vector with a nucleic acid sequence comprising the one or more open reading frames encoding the one or more peptides comprising the one or more immunogenic neo-epitopes or generating a second DNA immunotherapy vector or a second peptide immunotherapy vector using the nucleic acid sequence encoding the one or more peptides comprising the one or more immunogenic neo-epitopes identified in step (d); and
(g) alternatively (i) storing the second vector or the second DNA immunotherapy or the second peptide immunotherapy for administering to the subject at a pre-determined period, or administering a composition comprising the second vector, the second DNA immunotherapy, or the second peptide immunotherapy to the subject.

70. The process of any one of claim 68 or 69, wherein each of the one or more peptides comprising the one or more neo-epitopes is about 5-50 amino acids in length.

71. The process of claim 70, wherein each of the one or more peptides comprising the one or more neo-epitopes is about 8-27 amino acids in length.

72. The process of any one of claim 68 or 69, wherein the one or more neo-epitopes comprise 5-100 neo-epitopes.

73. The process of claim 72, wherein the one or more neo-epitopes comprise 15-35 neo-epitopes, 8-11 neo-epitopes or 11-16 neo-epitopes.

74. The process of any one of claims 68-73, wherein the one or more neo-epitopes comprise a plurality of neo-epitopes, wherein step (b) further comprises one or more iterations of randomizing the order of the one or more peptides comprising the plurality of neo-epitopes within the nucleic acid sequence of step (b).

75. The process of any one of claims 68-74, wherein the method is repeated to create a plurality of vectors, DNA immunotherapies, or peptide immunotherapies, each comprising a different set of one or more neo-epitopes.

76. The process of claim 75, wherein the plurality of vectors, DNA immunotherapies, or peptide immunotherapies comprises 5-10, 10-15, 15-20, 10-20, 20-30, 30-40, or 40-50 attenuated recombinant Listeria strains.

77. The process of claim 75 or 76, wherein the combination of the plurality of vectors, DNA immunotherapies, or peptide immunotherapies comprises about 5-10, 10-15, 15-20, 10-20, 20-30, 30-40, 40-50, 50-60, 60-70, 70-80, 80-90, 90-100, or 100-200 neo-epitopes.

78. The process of claim 77, wherein the comparing in step (a) comprises use of a screening assay or screening tool and associated digital software for comparing the one or more ORFs in the nucleic acid sequences extracted from the disease-bearing biological sample with the one or more ORFs in the nucleic acid sequences extracted from the healthy biological sample,
wherein the associated digital software comprises access to a sequence database that allows screening of mutations within the ORFs in the nucleic acid sequences extracted from the disease-bearing biological sample for identification of immunogenic potential of the neo-epitopes.

79. The process of any one of claims 69-78, wherein the process of obtaining a second biological sample from the subject in step (d) comprises obtaining a second biological sample comprising T-cell clones or T-infiltrating cells that expand following administration of the composition comprising the vector, the DNA immunotherapy, or the peptide immunotherapy.

80. The process of any one of claims 68-79, wherein the disease-bearing biological sample is tissue, cells, blood, or sera.

81. The process of any one of claims 69-80, wherein the process of characterizing in step (d) comprises the steps of:
(i) identifying, isolating, and expanding T cell clones or T-infiltrating cells that respond against the disease; and
(ii) screening for and identifying one or more peptides comprising one or more immunogenic neo-epitopes loaded on specific MHC Class I or MHC Class II molecules to which a T-cell receptor on the T cells binds.

82. The process of claim 81, wherein the screening for and identifying in step (ii) comprises T-cell receptor sequencing, multiplex based flow cytometry, or high-performance liquid chromatography.

83. The process of claim 82, wherein the sequencing comprises the use of associated digital software and database.

84. The process of any one of claims 68-83 wherein the disease or condition is an infectious disease, a tumor, or a cancer.

85. The process of claim 84, wherein the infectious disease comprises a viral or bacterial infection.

86. The process of any one of claims 68-86, wherein the healthy biological sample is obtained from the subject having the disease or condition.

87. The process of any one of claims 68-87, wherein the nucleic acid sequences extracted from the disease-bearing biological sample and the nucleic acid sequence extracted from the healthy biological sample are determined using exome sequencing or transcriptome sequencing.

89. The process of any one of claims 68-88, wherein the one or more neo-epitopes comprise linear neo-epitopes.

90. The process of any one of claims 68-89, wherein the one or more neo-epitopes comprise a solvent-exposed epitope.

91. The process of any one of claims 68-90, wherein the one or more neo-epitopes comprise a T-cell epitope.

92. The process of any one of claims 68-91, wherein the vector is a vaccinia virus or a virus-like particle.

93. The process of claim 92, wherein step (b) further comprises culturing and characterizing the vaccinia virus or virus-like particle to confirm expression of the one or more peptides.

94. The process of any one of claims 68-93, wherein the DNA immunotherapy comprises the nucleic acid sequence comprising the one or more peptides comprising the one or more immunogenic neo-epitopes.

95. The process of claim 94, wherein the nucleic acid sequence is in the form of a plasmid.

96. The process of any one of claims 68-95, wherein the plasmid is an integrative or an extrachrosomomal multicopy plasmid.

97. The process of any one of claims 68-96, wherein the one or more peptides comprising the one or more neo-epitopes are each fused to an immunogenic polypeptide or fragment thereof.

98. The process of any one of claims 68-96, wherein the peptide immunotherapy comprises the one or more peptides comprising the one or more neo-epitopes, wherein each peptide is fused to or mixed with an immunogenic polypeptide or fragment thereof.

99. The process of any one of claims 97-98, wherein the immunogenic polypeptide is a mutated Listeriolysin O (LLO) protein, a truncated LLO (tLLO) protein, a truncated ActA protein, or a PEST amino acid sequence.

100. The process of claim 99, wherein the tLLO protein is set forth in SEQ ID NO: 3.

101. The process of claim 99, wherein the actA is set forth in SEQ ID NO: 12-13 and 15-18.

102. The process of claim 99, wherein the PEST amino acid sequence is selected from the sequences set forth in SEQ ID NOs: 5-10.

103. The process of claim 99, wherein the mutated LLO comprises a mutation in a cholesterol-binding domain (CBD).

104. The process of claim 103, wherein the mutation comprises a substitution of residue C484, W491, or W492 of SEQ ID NO: 2, or any combination thereof.

105. The process of claim 103, wherein the mutation comprises a substitution of 1-11 amino acid within the CBD set forth in SEQ ID NO: 68 with a 1-50 amino acid non-LLO peptide, wherein the non-LLO peptide comprises a peptide comprising a neo-epitope.

106. The process of claim 103, wherein the mutation comprises a deletion of a 1-11 amino acid within the CBD as set forth in SEQ ID NO: 68.

107. The process of any one of claims 68-106, wherein the one or more peptides comprise a heterologous antigen or a self-antigen associated with the disease.

108. The process of claim 107, wherein the heterologous antigen or the self-antigen is a tumor-associated antigen or a fragment thereof.

109. The process of any one of claims 68-108, wherein the one or more neo-epitopes comprise a cancer-specific or tumor-specific epitope.

110. The process of any one of claims 108-109, wherein the tumor-associated antigen or fragment thereof comprises a Human Papilloma Virus (HPV)-16-E6, HPV-16-E7, HPV-18-E6, HPV-18-E7, a Her/2-neu antigen, a chimeric Her2 antigen, a Prostate Specific Antigen (PSA), bivalent PSA, ERG, Androgen receptor (AR), PAK6, Prostate Stem Cell Antigen (PSCA), NY-ESO-1, a Stratum Corneum Chymotryptic Enzyme (SCCE) antigen, Wilms tumor antigen 1 (WT-1), HIV-1 Gag, human telomerase reverse transcriptase (hTERT), Proteinase 3, Tyrosinase Related Protein 2 (TRP2), High Molecular Weight Melanoma Associated Antigen (HMW-MAA), synovial sarcoma, X (SSX)-2, carcinoembryonic antigen (CEA), Melanoma-Associated Antigen E (MAGE-A, MAGE 1, MAGE2, MAGE3, MAGE4), interleukin-13 Receptor alpha (IL13-R alpha), Carbonic anhydrase IX (CAIX), survivin, GP100, an angiogenic antigen, a ras protein, a p53 protein, a p97 melanoma antigen, KLH antigen, carcinoembryonic antigen (CEA), gp100, MART 1 antigen, TRP-2, HSP-70, beta-HCG, or Testisin.

111. The process of claim 84, wherein the tumor or cancer comprises a breast cancer or tumor, a cervical cancer or tumor, a Her2-expressing cancer or tumor, a melanoma, a pancreatic cancer or tumor, an ovarian cancer or tumor, a gastric cancer or tumor, a carcinomatous lesion of the pancreas, a pulmonary adenocarcinoma, a glioblastoma multiforme, a colorectal adenocarcinoma, a pulmonary squamous adenocarcinoma, a gastric adenocarcinoma, an ovarian surface epithelial neoplasm, an oral squamous cell carcinoma, non-small-cell lung carcinoma, an endometrial carcinoma, a bladder cancer or tumor, a head and neck cancer or tumor, a prostate carcinoma, a renal cancer or tumor, a bone cancer or tumor, a blood cancer, or a brain cancer or tumor.

112. The process of any one of claims 68-111, wherein the one or more neo-epitopes comprise an infectious-disease-associated epitope.

113. The process of claim 112, wherein the infectious disease is an infectious viral disease or an infectious bacterial disease.

114. The process of claim 113, wherein the infectious disease is caused by one of the following pathogens: Leishmania, Entamoeba histolytica (which causes amebiasis), Trichuris, BCG/Tuberculosis, Malaria, Plasmodium falciparum, Plasmodium malariae, Plasmodium vivax, Rotavirus, Cholera, Diptheria-Tetanus, Pertussis, Haemophilus influenzae, Hepatitis B, Human papilloma virus, Influenza seasonal), Influenza A (H1N1) Pandemic, Measles and Rubella, Mumps, Meningococcus A+C, Oral Polio Immunotherapies, mono, bi and trivalent, Pneumococcal, Rabies, Tetanus Toxoid, Yellow Fever, Bacillus anthracis (anthrax), Clostridium botulinum toxin (botulism), Yersinia pestis (plague), Variola major (smallpox) and other related pox viruses, Francisella tularensis (tularemia), Viral hemorrhagic fevers, Arenaviruses (LCM, Junin virus, Machupo virus, Guanarito virus, Lassa Fever), Bunyaviruses (Hantaviruses, Rift Valley Fever), Flaviruses (Dengue), Filoviruses (Ebola, Marburg), Burkholderia pseudomallei, Coxiella burnetii (Q fever), Brucella species (brucellosis), Burkholderia mallei (glanders), Chlamydia psittaci (Psittacosis), Ricin toxin (from Ricinus communis), Epsilon toxin of Clostridium perfringens, Staphylococcus enterotoxin B, Typhus fever (Rickettsia prowazekii), other Rickettsias, Food- and Waterborne Pathogens, Bacteria (Diarrheagenic E. coli, Pathogenic Vibrios, Shigella species, Salmonella BCG/, Campylobacter jejuni, Yersinia enterocolitica), Viruses (Caliciviruses, Hepatitis A, West Nile Virus, LaCrosse, Calif. encephalitis, VEE, EEE, WEE, Japanese Encephalitis Virus, Kyasanur Forest Virus, Nipah virus, hantaviruses, Tickborne hemorrhagic fever viruses, Chikungunya virus, Crimean-Congo Hemorrhagic fever virus, Tickborne encephalitis viruses, Hepatitis B virus, Hepatitis C virus, Herpes Simplex virus (HSV), Human immunodeficiency virus (HIV), Human papillomavirus (HPV)), Protozoa (Cryptosporidium parvum, Cyclospora cayatanensis, Giardia lamblia, Entamoeba histolytica, Toxoplasma), Fungi (Microsporidia), Yellow fever, Tuberculosis, including drug-resistant TB, Rabies, Prions, Severe acute respiratory syndrome associated coronavirus (SARS-CoV), Coccidioides posadasii, Coccidioides immitis, Bacterial vaginosis, Chlamydia trachomatis, Cytomegalovirus, Granuloma inguinale, Hemophilus ducreyi, Neisseria gonorrhea, Treponema pallidum, Streptococcus mutans, or Trichomonas vaginalis.

115. The process of any one of claims 68-114, wherein step (c)(ii) further comprises administering an adjuvant to the subject.

116. The process of claim 115, wherein the adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

117. The process of any one of claims 68-116, wherein step (c)(ii) further comprises administering an immune checkpoint inhibitor antagonist.

118. The process of claim 117, wherein the immune checkpoint inhibitor is an anti-PD-L1/PD-L2 antibody or fragment thereof, an anti-PD-1 antibody or fragment thereof, an anti-CTLA-4 antibody or fragment thereof, or an anti-B7-H4 antibody or fragment thereof.

119. The process of any one of claims 68-118, wherein the administering in step (c)(ii) generates a personalized enhanced anti-disease, or anti-condition immune response in said subject.

120. The process of claim 119, wherein the immune response comprises an anti-cancer or anti-tumor response.

121. The process of claim 119, wherein the immune response comprises an anti-infectious disease response.

122. The process of claim 121, wherein the infectious disease comprises a viral infection or bacterial infection.

123. The process of any one of claims 68-122, wherein the process allows personalized treatment or prevention of the disease or condition in the subject.

124. An immunogenic mixture of compositions comprising one or more attenuated recombinant Listeria strains produced by the process of any one of claims 68-123.

125. An immunogenic mixture of compositions comprising one or more attenuated recombinant Listeria strains, wherein each attenuated recombinant Listeria strain comprises a nucleic acid sequence encoding one or more peptides comprising one or more neo-epitopes present in a disease-bearing biological sample from a subject having a disease or condition.

126. The immunogenic mixture of claim 125, wherein the one or more attenuated recombinant Listeria strains comprise a plurality of attenuated recombinant Listeria strains, where the nucleic acid sequence in each attenuated recombinant Listeria strain encodes a different set of one or more neo-epitopes.

127. The immunogenic mixture of any one of claims 125-126, wherein the plurality of attenuated recombinant Listeria strains comprises 5-10, 10-15, 15-20, 10-20, 20-30, 30-40, or 40-50 attenuated recombinant Listeria strains.

128. The immunogenic mixture of any one of claims 125-127, wherein the combination of the plurality of attenuated recombinant Listeria strains comprises about 5-10, 10-15, 15-20, 10-20, 20-30, 30-40, 40-50, 50-60, 60-70, 70-80, 80-90, 90-100, or 100-200 neo-epitopes.

129. The immunogenic mixture of claim 128, wherein each of the attenuated recombinant Listeria strains in the mixture comprises a nucleic acid molecule encoding a fusion polypeptide or chimeric protein comprising one or more neo-epitopes.

130. The immunogenic mixture of compositions of claim 129, wherein each of the recombinant Listeria strains in the mixture expresses 1-20 neo-epitopes.

131. A method of eliciting a personalized anti-tumor response in a subject, the method comprising the step of concomitantly or sequentially administering to the subject the immunogenic mixture of any one of claims 128-131.

132. A method of preventing or treating a tumor in a subject, the method comprising concomitantly or sequentially administering to the subject the immunogenic mixture of any one of claims 125-130.

132. A nucleic acid construct encoding a chimeric protein comprising the following elements: an immunogenic polypeptide fused to a first neo-epitope amino acid sequence, wherein the first neo-epitope amino acid sequence is operatively linked to a second neo-epitope amino acid sequence via a first linker sequence, wherein the second neo-epitope amino acid sequence is operatively linked to at least one additional neo-epitope amino acid sequence via a second linker sequence.

133. A nucleic acid construct encoding a chimeric protein comprising the following elements: an N-terminal truncated LLO (tLLO) fused to a first neo-epitope amino acid sequence, wherein the first neo-epitope amino acid sequence is operatively linked to a second neo-epitope amino acid sequence via a first linker sequence, wherein the second neo-epitope amino acid sequence is operatively linked to at least one additional neo-epitope amino acid sequence via a second linker sequence, and wherein a last neo-epitope is operatively linked to a histidine tag at the C-terminus via a third linker sequence.

134. The nucleic acid construct of claim 133, wherein the first, the second, and the at least one addition neo-epitope amino acid sequences each about 5-50 amino acids.

135. The nucleic acid construct of claim 134, wherein the first, the second, and the at least one addition neo-epitope amino acid sequences each about 8-27 amino acids, 8-11 amino acids or 11-16 amino acids.

136. The nucleic acid construct of claim 135, wherein the first, the second, and the at least one addition neo-epitope amino acid sequences each comprises 21 amino acids.

137. The nucleic acid construct of any one of claims 132-136, wherein the nucleic acid construct encodes 5-100 neo-epitopes.

138. The nucleic acid construct of claim 137, wherein the nucleic acid construct encodes 15-35 neo-epitopes.

139. The nucleic acid construct of any one of claims 132-138, wherein the elements are arranged or are operatively linked from N-terminus to C-terminus.

140. The nucleic acid construct of any one of claims 137-139, wherein the tLLO is operatively linked to a promoter sequence.

141. The nucleic acid construct of claim 140, wherein the promoter sequence is an hly promoter sequence.

142. The nucleic acid construct of any one of claims 132-141, wherein the nucleic acid construct comprises 2 stop codons following the sequence encoding the histidine tag.

143. The nucleic acid construct of any one of claims 132-142, wherein the histidine tag is a 6× histidine tag that is operatively linked at the N-terminus to a SIINFEKL peptide.

144. The nucleic acid construct of any one of claims 132-143, wherein one or more of the first, second, and third linker sequences is a 4× glycine linker.

145. The nucleic acid construct of any one of claims 132-144, wherein the construct comprises the following components: pHly-tLLO-21mer #1-4× glycine linker G1-21mer #2-4× glycine linker G2- . . . -SIINFEKL-6×His tag-2× stop codon.

146. A chimeric protein encoded by the nucleic acid construct of any one of claims 132-145.

147. A recombinant Listeria strain comprising the nucleic acid construct of any one of claims 132-145 or expressing the chimeric protein of claim 146.


SEQUENCE LISTING (688 sequences)

SEQ ID NO: 1 - protein, 32 aa, Listeria monocytogenes
KENSISSMAPPASPPASPKTPIEKKHADEIDK

SEQ ID NO: 2 - protein, 529 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 3 - protein, 441 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 4 - protein, 416 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 5 - protein, 14 aa, Listeria monocytogenes
KTEEQPSEVNTGPR

SEQ ID NO: 6 - protein, 28 aa, Listeria monocytogenes
KASVTDTSEGDLDSSMQSADESTPQPLK

SEQ ID NO: 7 - protein, 20 aa, Listeria monocytogenes
KNEEVNASDFPPPPTDEELR

SEQ ID NO: 8 - protein, 33 aa, Listeria monocytogenes
RGGIPTSEEFSSLNSGDFTDDENSETTEEEIDR

SEQ ID NO: 9 - protein, 17 aa, Listeria monocytogenes
KQNTASTETTTTNEQPK

SEQ ID NO: 10 - protein, 17 aa, Listeria monocytogenes
KQNTANTETTTTNEQPK

SEQ ID NO: 11 - protein, 633 aa, Listeria monocytogenes
MRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSEEFSSLNSGDFTDDENSETTEEEIDRLADLRDRGTGKHSRNAGFLPLNPFISSPVPSLTPKVPKISAPALISDITKKAPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 12 - protein, 390 aa, Listeria monocytogenes
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 13 - protein, 100 aa, Listeria monocytogenes
MGLNRFMRAMMVVFITANCITINPDIIFAATDSEDSSLNTDEWEEEKTEEQPSEVNTGPRYETAREVSSRDIKELEKSNKVRNTNKADLIAMLKEKAEKG

SEQ ID NO: 14 - protein, 639 aa, Listeria monocytogenes
MGLNRFMRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSEEFSSLNSGDFTDDENSETTEEEIDRLADLRDRGTGKHSRNAGFLPLNPFISSPVPSLTPKVPKISAPALISDITKKAPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 15 - protein, 93 aa, Listeria monocytogenes
ATDSEDSSLNTDEWEEEKTEEQPSEVNTGPRYETAREVSSRDIEELEKSNKVKNTNKADLIAMLKAKAEKGPNNNNNNGEQTGNVAINEEASG

SEQ ID NO: 16 - protein, 200 aa, Listeria monocytogenes
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDK

SEQ ID NO: 17 - protein, 303 aa, Listeria monocytogenes
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSS

SEQ ID NO: 18 - protein, 370 aa, Listeria monocytogenes
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSE

SEQ ID NO: 19 - DNA, 1170 bp, Listeria monocytogenes
atgcgtgcgatgatggtggttttcattactgccaattgcattacgattaaccccgacataatatttgcagcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagacca

SEQ ID NO: 20 - protein, 98 aa, Listeria monocytogenes
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 21 - protein, 105 aa, Listeria monocytogenes
MHGPKATLQDIVLHLEPQNEIPVDLLCHEQLSDSEEENDEIDGVNHQHLPARRAEPQRHTMLCMCCKCEARIELVVESSADDLRAFQQLFLNTLSFVCPWCASQQ

SEQ ID NO: 22 - protein, 1263 aa, Listeria monocytogenes
GAGACCCACCTGGACATGCTCCGCCACCTCTACCAGGGCTGCCAGGTGGTGCAGGGAAACCTGGAACTCACCTACCTGCCCACCAATGCCAGCCTGTCCTTCCTGCAGGATATCCAGGAGGTGCAGGGCTACGTGCTCATCGCTCACAACCAAGTGAGGCAGGTCCCACTGCAGAGGCTGCGGATTGTGCGAGGCACCCAGCTCTTTGAGGACAACTATGCCCTGGCCGTGCTAGACAATGGAGACCCGCTGAACAATACCACCCCTGTCACAGGGGCCTCCCCAGGAGGCCTGCGGGAGCTGCAGCTTCGAAGCCTCACAGAGATCTTGAAAGGAGGGGTCTTGATCCAGCGGAACCCCCAGCTCTGCTACCAGGACACGATTTTGTGGAAGAATATCCAGGAGTTTGCTGGCTGCAAGAAGATCTTTGGGAGCCTGGCATTTCTGCCGGAGAGCTTTGATGGGGACCCAGCCTCCAACACTGCCCCGCTCCAGCCAGAGCAGCTCCAAGTGTTTGAGACTCTGGAAGAGATCACAGGTTACCTATACATCTCAGCATGGCCGGACAGCCTGCCTGACCTCAGCGTCTTCCAGAACCTGCAAGTAATCCGGGGACGAATTCTGCACAATGGCGCCTACTCGCTGACCCTGCAAGGGCTGGGCATCAGCTGGCTGGGGCTGCGCTCACTGAGGGAACTGGGCAGTGGACTGGCCCTCATCCACCATAACACCCACCTCTGCTTCGTGCACACGGTGCCCTGGGACCAGCTCTTTCGGAACCCGCACCAAGCTCTGCTCCACACTGCCAACCGGCCAGAGGACGAGTGTGTGGGCGAGGGCCTGGCCTGCCACCAGCTGTGCGCCCGAGGGCAGCAGAAGATCCGGAAGTACACGATGCGGAGACTGCTGCAGGAAACGGAGCTGGTGGAGCCGCTGACACCTAGCGGAGCGATGCCCAACCAGGCGCAGATGCGGATCCTGAAAGAGACGGAGCTGAGGAAGGTGAAGGTGCTTGGATCTGGCGCTTTTGGCACAGTCTACAAGGGCATCTGGATCCCTGATGGGGAGAATGTGAAAATTCCAGTGGCCATCAAAGTGTTGAGGGAAAACACATCCCCCAAAGCCAACAAAGAAATCTTAGACGAAGCATACGTGATGGCTGGTGTGGGCTCCCCATATGTCTCCCGCCTTCTGGGCATCTGCCTGACATCCACGGTGCAGCTGGTGACACAGCTTATGCCCTATGGCTGCCTCTTAGACTAA

SEQ ID NO: 23 - protein, 420 aa, Listeria monocytogenes [sequence omitted]

SEQ ID NO: 24 - DNA, 22 bp, Listeria monocytogenes
ggctcgagcatggagatacacc

SEQ ID NO: 25 - DNA, 28 bp, Listeria monocytogenes
ggggactagtttatggtttctgagaaca

SEQ ID NO: 26 - DNA, 31 bp, Listeria monocytogenes
gggggctagccctcctttgattagtatattc

SEQ ID NO: 27 - DNA, 28 bp, Listeria monocytogenes
ctccctcgagatcataatttacttcatc

SEQ ID NO: 28 - DNA, 55 bp, Listeria monocytogenes
gactacaaggacgatgaccgacaagtgataacccgggatctaaataaatccgttt

SEQ ID NO: 29 - DNA, 27 bp, Listeria monocytogenes
cccgtcgaccagctcttcttggtgaag

SEQ ID NO: 30 - DNA, 25 bp, Listeria monocytogenes
gcggatcccatggagatacacctac

SEQ ID NO: 31 - DNA, 22 bp, Listeria monocytogenes
gctctagattatggtttctgag

SEQ ID NO: 32 - DNA, 31 bp, Listeria monocytogenes
ggggtctagacctcctttgattagtatattc

SEQ ID NO: 33 - DNA, 45 bp, Listeria monocytogenes
atcttcgctatctgtcgccgcggcgcgtgcttcagtttgttgcgc

SEQ ID NO: 34 - DNA, 45 bp, Listeria monocytogenes
gcgcaacaaactgaagcagcggccgcggcgacagatagcgaagat

SEQ ID NO: 35 - DNA, 42 bp, Listeria monocytogenes
tgtaggtgtatctccatgctcgagagctaggcgatcaatttc

SEQ ID NO: 36 - DNA, 42 bp, Listeria monocytogenes
ggaattgatcgcctagctctcgagcatggagatacacctaca

SEQ ID NO: 37 - DNA, 42 bp, Listeria monocytogenes
aaacggatttatttagatcccgggttatggtttctgagaaca

SEQ ID NO: 38 - DNA, 42 bp, Listeria monocytogenes
tgttctcagaaaccataacccgggatctaaataaatccgttt

SEQ ID NO: 39 - DNA, 28 bp, Listeria monocytogenes
gggggtcgaccagctcttcttggtgaag

SEQ ID NO: 40 - protein, 9 aa, Listeria monocytogenes
RAHYNIVTF

SEQ ID NO: 41 - DNA, 6523 bp, Escherichia coli
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaacccctaacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcgcggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 42 - DNA, 36 bp, Listeria monocytogenes
cggaattcggatccgcgccaaatcattggttgattg

SEQ ID NO: 43 - DNA, 37 bp, Listeria monocytogenes
gcgagtcgacgtcggggttaatcgtaatgcaattggc

SEQ ID NO: 44 - DNA, 35 bp, Listeria monocytogenes
gcgagtcgacccatacgacgttaattcttgcaatg

SEQ ID NO: 45 - DNA, 39 bp, Listeria monocytogenes
gatactgcagggatccttcccttctcggtaatcagtcac

SEQ ID NO: 46 - DNA, 19 bp, Listeria monocytogenes
tgggatggccaagaaattc

SEQ ID NO: 47 - DNA, 22 bp, Listeria monocytogenes
ctaccatgtcttccgttgcttg

SEQ ID NO: 48 - DNA, 28 bp, artificial
  Human/e-coli chimera
tgatctcgagacccacctggacatgctc

SEQ ID NO: 49 - DNA, 49 bp, Artificial Sequence
  human/e. coli chimera
ctaccaggacacgattttgtggaagaatatccaggagtttgctggctgc

SEQ ID NO: 50 - DNA, 49 bp, artificial
  human/e. coli chimera
gcagccagcaaactcctggatattcttccacaaaatcgtgtcctggtag

SEQ ID NO: 51 - DNA, 50 bp, artificial
  human/e. coli chimera
ctgccaccagctgtgcgcccgagggcagcagaagatccggaagtacacga

SEQ ID NO: 52 - DNA, 39 bp, artificial
  human/e. coli chimera
gtggcccgggtctagattagtctaagaggcagccatagg

SEQ ID NO: 53 - DNA, 31 bp, artificial
  human/e. coli chimera
cgcgactagtttaatcctctgctgtcacctc

SEQ ID NO: 54 - DNA, 31 bp, artificial; identical to SEQ ID NO: 53
  human/e. coli chimera

SEQ ID NO: 55 - DNA, 28 bp, artificial
  human/e. coli chimera
ccgcctcgagtacctttctacggacgtg

SEQ ID NO: 56 - DNA, 30 bp, artificial
  human/e. coli chimera
cgcgactagtttactctggccggttggcag

SEQ ID NO: 57 - DNA, 31 bp, artificial
  human/e. coli chimera
ccgcctcgagcagcagaagatccggaagtac

SEQ ID NO: 58 - DNA, 30 bp, artificial
  human/e. coli chimera
cgcgactagtttaagccccttcggagggtg

SEQ ID NO: 59 - protein, 9 aa, homo sapiens
HLYQGCQVV

SEQ ID NO: 60 - protein, 9 aa, homo sapiens
KIFGSLAFL

SEQ ID NO: 61 - protein, 9 aa, homo sapiens
RLLQETELV

SEQ ID NO: 62 - protein, 535 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIEHHHHHH

SEQ ID NO: 63 - DNA, 1551 bp, Listeria monocytogenes
catatgaaggatgcatctgcattcaataaagaaaattcaatttcatccgtggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttattcaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatcctgaaggtaacgaaattgttcaacataaaaactggagcgaaaacaataaaagcaagctagctcatttcacatcgtccatctatttgcctggtaacgcgagaaatattaatgtttacgctaaagaatgcactggtttagcttgggaatggtggagaacggtaattgatgaccggaacttaccacttgtgaaaaatagaaatatctccatctggggcaccacgctttatccgaaatatagtaataaagtagataatccaatcgaacaccaccaccaccaccactaataaggatcc

SEQ ID NO: 64 - DNA, 20 bp, Listeria monocytogenes
gctagctcatttcacatcgt

SEQ ID NO: 65 - DNA, 54 bp, Listeria monocytogenes
tcttgcagcttcccaagctaaaccagtcgcttctttagcgtaaacattaatatt

SEQ ID NO: 66 - DNA, 54 bp, Listeria monocytogenes
gaagcgactggtttagcttgggaagctgcaagaacggtaattgatgaccggaac

SEQ ID NO: 67 - DNA, 39 bp, Listeria monocytogenes
ggatccttattagtggtggtggtggtggtgttcgattgg

SEQ ID NO: 68 - protein, 11 aa, Listeria monocytogenes
ECTGLAWEWWR

SEQ ID NO: 69 - protein, 11 aa, Listeria monocytogenes
EATGLAWEAAR

SEQ ID NO: 70 - DNA, 238 bp, Listeria monocytogenes
gctagctcatttcacatcgtccatctatttgcctggtaacgcgagaaatattaatgtttacgctaaagaagcgactggtttagcttgggaagctgcaagaacggtaattgatgaccggaacttaccacttgtgaaaaatagaaatatctccatctggggcaccacgctttatccgaaatatagtaataaagtagataatccaatcgaacaccaccaccaccaccactaataaggatcc

SEQ ID NO: 71 - protein, 11 aa, Listeria monocytogenes
ESLLMWITQCR

SEQ ID NO: 72 - DNA, 54 bp, Listeria monocytogenes
tctgcactgggtgatccacatcagcaggctttctttagcgtaaacattaatatt

SEQ ID NO: 73 - DNA, 54 bp, Listeria monocytogenes
gaaagcctgctgatgtggatcacccagtgcagaacggtaattgatgaccggaac

SEQ ID NO: 74 - DNA, 238 bp, Listeria monocytogenes
gctagctcatttcacatcgtccatctatttgcctggtaacgcgagaaatattaatgtttacgctaaagaaagcctgctgatgtggatcacccagtgcagaacggtaattgatgaccggaacttaccacttgtgaaaaatagaaatatctccatctggggcaccacgctttatccgaaatatagtaataaagtagataatccaatcgaacaccaccaccaccaccactaataaggatcc

SEQ ID NO: 75 - protein, 8 aa, Listeria monocytogenes
SIINFEKL

SEQ ID NO: 76 - DNA, 12 bp, artificial
  linker
ggtggtggagga

SEQ ID NO: 77 - DNA, 12 bp, artificial
  linker
ggtggaggtgga

SEQ ID NO: 78 - DNA, 12 bp, artificial
  linker
ggtggaggaggt

SEQ ID NO: 79 - DNA, 12 bp, artificial
  linker
ggaggtggtgga

SEQ ID NO: 80 - DNA, 12 bp, artificial
  linker
ggaggaggtggt

SEQ ID NO: 81 - DNA, 12 bp, artificial
  linker
ggaggtggaggt

SEQ ID NO: 82 - DNA, 12 bp, artificial
  linker
ggaggaggaggt

SEQ ID NO: 83 - DNA, 12 bp, artificial
  linker
ggaggaggtgga

SEQ ID NO: 84 - DNA, 12 bp, artificial
  linker
ggaggtggagga

SEQ ID NO: 85 - DNA, 12 bp, artificial
  linker
ggtggaggagga

SEQ ID NO: 86 - DNA, 12 bp, artificial
  linker
ggaggaggagga

SEQ ID NO: 87 - protein, 17 aa, Listeria monocytogenes
ARSIINFEKLSHHHHHH

SEQ ID NO: 88 - protein, 20 aa, Listeria monocytogenes
FMVAVAHVAAFLLEDRAVCV

SEQ ID NO: 89 - protein, 21 aa, Listeria monocytogenes
AENVEQVLVTSIQGAVDYPDP

SEQ ID NO: 90 - protein, 21 aa, Listeria monocytogenes
SFKKKFEECQHNIIKLQNGHT

SEQ ID NO: 91 - protein, 21 aa, Listeria monocytogenes
SALIESLNQKTQSTGDHPQPT

SEQ ID NO: 92 - protein, 21 aa, Listeria monocytogenes
KAYLPVNESFAFTADLRSNTG

SEQ ID NO: 93 - protein, 21 aa, Listeria monocytogenes
HTLLEITEESGAVLVDKSDSD

SEQ ID NO: 94 - protein, 21 aa, Listeria monocytogenes
SVMCTYSPPLDKLFCQLAKTC

SEQ ID NO: 95 - protein, 21 aa, Listeria monocytogenes
ESGKHKYRQTAMFTATMPPAV

SEQ ID NO: 96 - protein, 21 aa, Listeria monocytogenes
AAPSAASSPADVQSLKKAMSS

SEQ ID NO: 97 - protein, 21 aa, Listeria monocytogenes
SQLFSLNPRGRSLVTAGRIDR

SEQ ID NO: 98 - protein, 21 aa, Listeria monocytogenes
SLARGPLSEAGLALFDPYSKE

SEQ ID NO: 99 - protein, 21 aa, Listeria monocytogenes
QKKLCHLSSTGLPRETIASLP

SEQ ID NO: 100 - protein, 21 aa, Listeria monocytogenes
LTASNMEGKSWPSEVLVCTTS

SEQ ID NO: 101 - protein, 21 aa, Listeria monocytogenes
YAAQQHETFLTNGDRAGFLIG

SEQ ID NO: 102 - protein, 21 aa, Listeria monocytogenes
QAKVPFSEETQNLILPYISDM

SEQ ID NO: 103 - protein, 21 aa, Listeria monocytogenes
CNRAGEKHCFSSNEAARDFGG

SEQ ID NO: 104 - protein, 21 aa, Listeria monocytogenes
RNPQFLDPVLAYLMKGLCEKP

SEQ ID NO: 105 - protein, 21 aa, Listeria monocytogenes
LECERGKQEAKLLAERSRFED

SEQ ID NO: 106 - protein, 21 aa, Listeria monocytogenes
APLEWLRYFDKKELELMLCGM

SEQ ID NO: 107 - protein, 21 aa, Listeria monocytogenes
KAFLHWYTGEAMDEMEFTEAE

SEQ ID NO: 108 - protein, 21 aa, Listeria monocytogenes
DEVALVEGVQSLGFTYLRLKD

SEQ ID NO: 109 - protein, 21 aa, Listeria monocytogenes
DFSQLQRNILPSNPRVTRFHI

SEQ ID NO: 110 - protein, 21 aa, Listeria monocytogenes
ISTNGSFIRLLDAFKGVVMHT

SEQ ID NO: 111 - protein, 21 aa, Listeria monocytogenes
ITPPTTTTKKARVSTPKPATP

SEQ ID NO: 112 - protein, 21 aa, Listeria monocytogenes
NYNTSHLNNDVWQIFENPVDW

SEQ ID NO: 113 - protein, 21 aa, Listeria monocytogenes
QKTLHNLLRKVVPSFSAEIER

SEQ ID NO: 114 - protein, 21 aa, Listeria monocytogenes
VELCPGNKYEMRRHGTTHSLV

SEQ ID NO: 115 - protein, 21 aa, Listeria monocytogenes
GIDKLTQLKKPFLVNNKINKI

SEQ ID NO: 116 - protein, 20 aa, Listeria monocytogenes
GTTILNCFHDVLSGKLSGGS

SEQ ID NO: 117 - protein, 21 aa, Listeria monocytogenes
PSFQEFVDWENVSPELNSTDQ

SEQ ID NO: 118 - protein, 21 aa, Listeria monocytogenes
PALVEEYLERGNFVANDLDWL

SEQ ID NO: 119 - protein, 21 aa, Listeria monocytogenes
ELKACKPNGKRNPYCEVSMGS

SEQ ID NO: 120 - protein, 21 aa, Listeria monocytogenes
SPFPAAVILRDALHMARGLKY

SEQ ID NO: 121 - protein, 21 aa, Listeria monocytogenes
QQLDTYILKNVVAFSRTDKYR

SEQ ID NO: 122 - protein, 21 aa, Listeria monocytogenes
SFVGQTRVLMINGEEVEETEL

SEQ ID NO: 123 - protein, 21 aa, Listeria monocytogenes
AFFINFIAIYHHASRAIPFGT

SEQ ID NO: 124 - protein, 21 aa, Listeria monocytogenes
GLALPNNYCDVCLGDSKINKK

SEQ ID NO: 125 - protein, 21 aa, Listeria monocytogenes
EGQISIAKYENCPKDNPMYYC

SEQ ID NO: 126 - protein, 21 aa, Listeria monocytogenes
NFKRKRVAAFQKNLIEMSELE

SEQ ID NO: 127 - protein, 21 aa, Listeria monocytogenes
KMKGELGMMLILQNVIQKTTT

SEQ ID NO: 128 - protein, 21 aa, Listeria monocytogenes
SIECKGIDKEINESKNTHLDI

SEQ ID NO: 129 - protein, 21 aa, Listeria monocytogenes
ELEAAIETVVCTFFTFAGREG

SEQ ID NO: 130 - protein, 21 aa, Listeria monocytogenes
SLSHREREQMKATLNYEDHCF

SEQ ID NO: 131 - protein, 21 aa, Listeria monocytogenes
HIKAFDRTFANNPGPMVVFAT

SEQ ID NO: 132 - protein, 21 aa, Listeria monocytogenes
ITSNFVIPSEYWVEEKEEKQK

SEQ ID NO: 133 - protein, 21 aa, Listeria monocytogenes
GLVTFQAFIDVMSRETTDTDT

SEQ ID NO: 134 - protein, 21 aa, Listeria monocytogenes
HLLGRLAAIVGKQVLLGRKVV

SEQ ID NO: 135 - protein, 21 aa, Listeria monocytogenes
SMDHKTGTIAMQNTTQLRSRY

SEQ ID NO: 136 - protein, 21 aa, Listeria monocytogenes
SMDHKTGTIAMQNTTQLRSRY

SEQ ID NO: 137 - protein, 21 aa, Listeria monocytogenes
QPLRRLVLHVVSAAQAERLAR

SEQ ID NO: 138 - DNA, 5851 bp, Listeria monocytogenes
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgaggagctcctgcagtctagagtcgacactagtggatccagatctcccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcacggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 139 - DNA, 678 bp, Listeria monocytogenes
atgaaaaaaataatgctagtttttattacacttatattagttagtctaccaattgcgcaacaaactgaagcatctagagcgacagatagcgaagattccagtctaaacacagatgaatgggaagaagaaaaaacagaagagcagccaagcgaggtaaatacgggaccaagatacgaaactgcacgtgaagtaagttcacgtgatattgaggaactagaaaaatcgaataaagtgaaaaatacgaacaaagcagacctaatagcaatgttgaaagcaaaagcagagaaaggtccgaataacaataataacaacggtgagcaaacaggaaatgtggctataaatgaagaggcttcaggagtcgaccgaccaactctgcaagtggagcgtcgtcatccaggtctgtcatcggatagcgcagcggaaattaaaaaaagaagaaaagccatagcgtcgtcggatagtgagcttgaaagccttacttatccagataaaccaacaaaagcaaataagagaaaagtggcgaaagagtcagttgtggatgcttctgaaagtgacttagattctagcatgcagtcagcagacgagtctacaccacaacctttaaaagcaaatcaaaaaccatttttccctaaagtatttaaaaaaataaaagatgcggggaaatgggtacgtgataaa

SEQ ID NO: 140 - protein, 21 aa, Homo sapiens
TTSKDWHHGGPENILLCTDCR

SEQ ID NO: 141 - DNA, 63 bp, Homo sapiens
acaacatctaaagattggcatcatggtggtccagaaaacattttattatgtacagattgtcgt

SEQ ID NO: 142 - protein, 21 aa, Homo sapiens
RYNPSLKGLNVTGYETQAGEF

SEQ ID NO: 143 - DNA, 63 bp, Homo sapiens
cgttataacccatctttaaaaggtttaaacgttacaggttatgaaacacaagcaggtgaattc

SEQ ID NO: 144 - protein, 21 aa, Homo sapiens
VSRAAVHCALSSPPPEARALA

SEQ ID NO: 145 - DNA, 63 bp, Homo sapiens
gtttctcgtgcagcagttcattgtgcattatcttctccaccaccagaagcacgtgcattagca

SEQ ID NO: 146 - protein, 21 aa, Homo sapiens
VQLRDGDSNCQWSVLYVIIAT

SEQ ID NO: 147 - DNA, 63 bp, Homo sapiens
gttcaattacgtgatggtgattctaactgtcaatggtctgttttatatgttattattgcaaca

SEQ ID NO: 148 - protein, 21 aa, Homo sapiens
EMNKQISLLTFAVNHLKANVK

SEQ ID NO: 149 - DNA, 63 bp, Homo sapiens
gaaatgaacaaacaaatttctttattaacattcgcagttaaccatttaaaagcaaacgttaaa

SEQ ID NO: 150 - protein, 21 aa, Homo sapiens
AWQNSIRHNLCLNDCFVKIPR

SEQ ID NO: 151 - DNA, 63 bp, Homo sapiens
gcatggcaaaactctattcgtcataacttatgtttaaacgattgtttcgttaaaattccacgt

SEQ ID NO: 152 - protein, 21 aa, Homo sapiens
DGLPGPPGTMMMLPFRYGGDG

SEQ ID NO: 153 - DNA, 63 bp, Homo sapiens
gatggtttaccaggtccaccaggtacaatgatgatgttaccattccgttatggtggtgatggt

SEQ ID NO: 154 - protein, 21 aa, Homo sapiens
ITVHTPLLPSATGLLNDNTFA

SEQ ID NO: 155 - DNA, 63 bp, Homo sapiens
attacagttcatacaccattattaccatctgcaacaggtttattaaacgataacacattcgca

SEQ ID NO: 156 - protein, 21 aa, Homo sapiens
AGEASRLAHYSKRSTITSREI

SEQ ID NO: 157 - DNA, 63 bp, Homo sapiens
gcaggtgaagcatctcgtttagcacattattctaaacgttctacaattacatctcgtgaaatt

SEQ ID NO: 158 - protein, 21 aa, Homo sapiens
GEVLPTPGQEPPTPIHDLSAS

SEQ ID NO: 159 - DNA, 63 bp, Homo sapiens
ggtgaagttttaccaacaccaggtcaagaaccaccaacaccaattcatgatttatctgcatct

SEQ ID NO: 160 - protein, 21 aa, Homo sapiens
ERTTEEVARNQRAVEAEKCAR

SEQ ID NO: 161 - DNA, 63 bp, Homo sapiens
gaacgtacaacagaagaagttgcacgtaaccaacgtgcagttgaagcagaaaaatgtgcacgt

SEQ ID NO: 162 - protein, 21 aa, Homo sapiens
SFPAQDPCTTTYVAATEPVPE

SEQ ID NO: 163 - DNA, 63 bp, Homo sapiens
tctttcccagcacaagatccatgtacaacaacatatgttgcagcaacagaaccagttccagaa

SEQ ID NO: 164 - protein, 21 aa, Homo sapiens
PSVPLGGAPSCGMLMDKPHPP

SEQ ID NO: 165 - DNA, 63 bp, Homo sapiens
ccatctgttccattaggtggtgcaccatcttgtggtatgttaatggataaaccacatccacca

SEQ ID NO: 166 - protein, 21 aa, Homo sapiens
SLSVKIRVMPCSFFLLLRFFL

SEQ ID NO: 167 - DNA, 63 bp, Homo sapiens
tctttatctgttaaaattcgtgttatgccatgttctttcttcttattattacgtttcttctta

SEQ ID NO: 168 - protein, 21 aa, Homo sapiens
SKQQVADEDEMWKQRRRQQSE

SEQ ID NO: 169 - DNA, 63 bp, Homo sapiens
tctaaacaacaagttgcagatgaagatgaaatgtggaaacaacgtcgtcgtcaacaatctgaa

SEQ ID NO: 170 - protein, 21 aa, Homo sapiens
PPLPDVSAFSSGRGGGEGRGS

SEQ ID NO: 171 - DNA, 63 bp, Homo sapiens
ccaccattaccagatgtttctgcattctcttctggtcgtggtggtggtgaaggtcgtggttct

SEQ ID NO: 172 - protein, 21 aa, Homo sapiens
NQPRLIQCENKVGKLLFITEI

SEQ ID NO: 173 - DNA, 63 bp, Homo sapiens
aaccaaccacgtttaattcaatgtgaaaacaaagttggtaaattattattcattacagaaatt

SEQ ID NO: 174 - protein, 21 aa, Homo sapiens
KSTTKRSPKPQNKKKTKKVIE

SEQ ID NO: 175 - DNA, 63 bp, Homo sapiens
aaatctacaacaaaacgttctccaaaaccacaaaacaaaaaaaaaacaaaaaaagttattgaa

SEQ ID NO: 176 - protein, 21 aa, Homo sapiens
KRKDSRTVYQSQYTNWSVEQL

SEQ ID NO: 177 - DNA, 63 bp, Homo sapiens
aaacgtaaagattctcgtacagtttatcaatctcaatatacaaactggtctgttgaacaatta

SEQ ID NO: 178 - protein, 21 aa, Homo sapiens
NLQFEYIDDPWVQRIEPEWSI

SEQ ID NO: 179 - DNA, 63 bp, Homo sapiens
aacttacaattcgaatatattgatgatccatgggttcaacgtattgaaccagaatggtctatt

SEQ ID NO: 180 - protein, 21 aa, Homo sapiens
ELMDKPLTFRILSDLIFDFED

SEQ ID NO: 181 - DNA, 63 bp, Homo sapiens
gaattaatggataaaccattaacattccgtattttatctgatttaattttcgatttcgaagat

SEQ ID NO: 182 - protein, 21 aa, Homo sapiens
QACTSGGCLHILPITVTTAQA

SEQ ID NO: 183 - DNA, 63 bp, Homo sapiens
caagcatgtacatctggtggttgtttacatattttaccaattacagttacaacagcacaagca

SEQ ID NO: 184 - protein, 21 aa, Homo sapiens
VEDERQMDDLQGAAPPIPPRL

SEQ ID NO: 185 - DNA, 63 bp, Homo sapiens
gttgaagatgaacgtcaaatggatgatttacaaggtgcagcaccaccaattccaccacgttta

SEQ ID NO: 186 - protein, 21 aa, Homo sapiens
TSNYTGFANKDGSKPSSLGVS

SEQ ID NO: 187 - DNA, 63 bp, Homo sapiens
acatctaactatacaggtttcgcaaacaaagatggttctaaaccatcttctttaggtgtttct

SEQ ID NO: 188 - protein, 21 aa, Homo sapiens
KLGISFSEDQKKEKDQLGKAP

SEQ ID NO: 189 - DNA, 63 bp, Homo sapiens
aaattaggtatttctttctctgaagatcaaaaaaaagaaaaagatcaattaggtaaagcacca

SEQ ID NO: 190 - protein, 21 aa, Homo sapiens
AAPFKAPELLKGQSEDEQPDA

SEQ ID NO: 191 - DNA, 63 bp, Homo sapiens
gcagcaccattcaaagcaccagaattattaaaaggtcaatctgaagatgaacaaccagatgca

SEQ ID NO: 192 - protein, 21 aa, Homo sapiens
ATNQASTKSHKYSKVTNSLSL

SEQ ID NO: 193 - DNA, 63 bp, Homo sapiens
gcaacaaaccaagcatctacaaaatctcataaatattctaaagttacaaactctttatcttta

SEQ ID NO: 194 - protein, 21 aa, Homo sapiens
VSNLEELPLNQYREYIKVQTD

SEQ ID NO: 195 - DNA, 63 bp, Homo sapiens
gtttctaacttagaagaattaccattaaaccaatatcgtgaatatattaaagttcaaacagat

SEQ ID NO: 196 - protein, 21 aa, Homo sapiens
AGCQLWLCTLLAVLKVGWLFP

SEQ ID NO: 197 - DNA, 63 bp, Homo sapiens
gcaggttgtcaattatggttatgtacattattagcagttttaaaagttggttggttattccca

SEQ ID NO: 198 - protein, 21 aa, Homo sapiens
PSRPSDAADEVRDTSINESRN

SEQ ID NO: 199 - DNA, 63 bp, Homo sapiens
ccatctcgtccatctgatgcagcagatgaagttcgtgatacatctattaacgaatctcgtaac

SEQ ID NO: 200 - protein, 21 aa, Homo sapiens
TYHSTYLVNQSAFDQRVATVH

SEQ ID NO: 201 - DNA, 63 bp, Homo sapiens
acatatcattctacatatttagttaaccaatctgcattcgatcaacgtgttgcaacagttcat

SEQ ID NO: 202 - protein, 21 aa, Homo sapiens
FCGGALIHPCRVLTAAHCTDI

SEQ ID NO: 203 - DNA, 63 bp, Homo sapiens
ttctgtggtggtgcattaattcatccatgtcgtgttttaacagcagcacattgtacagatatt

SEQ ID NO: 204 - protein, 21 aa, Homo sapiens
MAFVYLRVFRKAQKQVKKIDS

SEQ ID NO: 205 - DNA, 63 bp, Homo sapiens
atggcattcgtttatttacgtgttttccgtaaagcacaaaaacaagttaaaaaaattgattct

SEQ ID NO: 206 - protein, 21 aa, Homo sapiens
TTTLREWSTICRQLYVQDNRE

SEQ ID NO: 207 - DNA, 63 bp, Homo sapiens
acaacaacattacgtgaatggtctacaatttgtcgtcaattatatgttcaagataaccgtgaa

SEQ ID NO: 208 - protein, 21 aa, Homo sapiens
SQSSCCKPCCCQSSCCKPCCC

SEQ ID NO: 209 - DNA, 63 bp, Homo sapiens
tctcaatcttcttgttgtaaaccatgttgttgtcaatcttcttgttgtaaaccatgttgttgt

SEQ ID NO: 210 - protein, 21 aa, Homo sapiens
LGNGTILWIIVLQPALHRPMH

SEQ ID NO: 211 - DNA, 63 bp, Homo sapiens
ttaggtaacggtacaattttatggattattgttttacaaccagcattacatcgtccaatgcat

SEQ ID NO: 212 - protein, 21 aa, Homo sapiens
LVLAVSMTKTKNEVCGYPVIV

SEQ ID NO: 213 - DNA, 63 bp, Homo sapiens
ttagttttagcagtttctatgacaaaaacaaaaaacgaagtttgtggttatccagttattgtt

SEQ ID NO: 214 - protein, 21 aa, Homo sapiens
FSSNVTPKMLQIFLSEKKSIS

SEQ ID NO: 215 - DNA, 63 bp, Homo sapiens
ttctcttctaacgttacaccaaaaatgttacaaattttcttatctgaaaaaaaatctatttct

SEQ ID NO: 216 - protein, 21 aa, Homo sapiens
NYTILMRPRFVILLTVISWFL

SEQ ID NO: 217 - DNA, 63 bp, Homo sapiens
aactatacaattttaatgcgtccacgtttcgttattttattaacagttatttcttggttctta

SEQ ID NO: 218 - protein, 21 aa, Homo sapiens
IVLPSNVINFCQAEKPEPTNQ

SEQ ID NO: 219 - DNA, 63 bp, Homo sapiens
attgttttaccatctaacgttattaacttctgtcaagcagaaaaaccagaaccaacaaaccaa

SEQ ID NO: 220 - protein, 21 aa, Homo sapiens
GNMAVIHSHLLKGLQEKFLKG

SEQ ID NO: 221 - DNA, 63 bp, Homo sapiens
ggtaacatggcagttattcattctcatttattaaaaggtttacaagaaaaattcttaaaaggt

SEQ ID NO: 222 - protein, 21 aa, Homo sapiens
APEVIKASTFCKGSDVWSFGV

SEQ ID NO: 223 - DNA, 63 bp, Homo sapiens
gcaccagaagttattaaagcatctacattctgtaaaggttctgatgtttggtctttcggtgtt

SEQ ID NO: 224 - protein, 21 aa, Homo sapiens
AFHAFAQPFALPHSAMLFVQA

SEQ ID NO: 225 - DNA, 63 bp, Homo sapiens
gcattccatgcattcgcacaaccattcgcattaccacattctgcaatgttattcgttcaagca

SEQ ID NO: 226 - protein, 21 aa, Homo sapiens
LSVSRPERPPQSAALGGLGPG

SEQ ID NO: 227 - DNA, 63 bp, Homo sapiens
ttatctgtttctcgtccagaacgtccaccacaatctgcagcattaggtggtttaggtccaggt

SEQ ID NO: 228 - protein, 21 aa, Homo sapiens
VALELNGGLCVEACGALEGLW

SEQ ID NO: 229 - DNA, 63 bp, Homo sapiens
gttgcattagaattaaacggtggtttatgtgttgaagcatgtggtgcattagaaggtttatgg

SEQ ID NO: 230 - protein, 21 aa, Homo sapiens
KILQLCGELYHLDASSLQLKV

SEQ ID NO: 231 - DNA, 63 bp, Homo sapiens
aaaattttacaattatgtggtgaattatatcatttagatgcatcttctttacaattaaaagtt

SEQ ID NO: 232 - protein, 21 aa, Homo sapiens
PKLLRELVPQYLIRQVSPDDW

SEQ ID NO: 233 - DNA, 63 bp, Homo sapiens
ccaaaattattacgtgaattagttccacaatatttaattcgtcaagtttctccagatgattgg

SEQ ID NO: 234 - protein, 21 aa, Homo sapiens
HALMTYSTARMSFVCPRFYRN

SEQ ID NO: 235 - DNA, 63 bp, Homo sapiens
catgcattaatgacatattctacagcacgtatgtctttcgtttgtccacgtttctatcgtaac

SEQ ID NO: 236 - protein, 21 aa, Homo sapiens
PGQRWVWMNFQSPSHSPGLRS

SEQ ID NO: 237 - DNA, 63 bp, Homo sapiens
ccaggtcaacgttgggtttggatgaacttccaatctccatctcattctccaggtttacgttct

SEQ ID NO: 238 - protein, 21 aa, Homo sapiens
VPNLHWLVVENAPRRTPLTAR

SEQ ID NO: 239 - DNA, 63 bp, Homo sapiens
gttccaaacttacattggttagttgttgaaaacgcaccacgtcgtacaccattaacagcacgt

SEQ ID NO: 240 - protein, 21 aa, Homo sapiens
KLSSNRERHVTMCEDLELRRD

SEQ ID NO: 241 - DNA, 63 bp, Homo sapiens
aaattatcttctaaccgtgaacgtcatgttacaatgtgtgaagatttagaattacgtcgtgat

SEQ ID NO: 242 - protein, 21 aa, Homo sapiens
LLAVTGGCFMELCKVAKSVEM

SEQ ID NO: 243 - DNA, 63 bp, Homo sapiens
ttattagcagttacaggtggttgtttcatggaattatgtaaagttgcaaaatctgttgaaatg

SEQ ID NO: 244 - protein, 21 aa, Homo sapiens
QSSSRANFVIALINIPAVALG

SEQ ID NO: 245 - DNA, 63 bp, Homo sapiens
caatcttcttctcgtgcaaacttcgttattgcattaattaacattccagcagttgcattaggt

SEQ ID NO: 246 - protein, 21 aa, Homo sapiens
GYHDNGMFSPIGESCEDIDEC

SEQ ID NO: 247 - DNA, 63 bp, Homo sapiens
ggttatcatgataacggtatgttctctccaattggtgaatcttgtgaagatattgatgaatgt

SEQ ID NO: 248 - protein, 21 aa, Homo sapiens
GISTRLPGPGKPVPGPAGPAQ

SEQ ID NO: 249 - DNA, 63 bp, Homo sapiens
ggtatttctacacgtttaccaggtccaggtaaaccagttccaggtccagcaggtccagcacaa

SEQ ID NO: 250 - protein, 21 aa, Homo sapiens
ILTQLHQDMQQLEIPQAYRGA

SEQ ID NO: 251 - DNA, 63 bp, Homo sapiens
attttaacacaattacatcaagatatgcaacaattagaaattccacaagcatatcgtggtgca

SEQ ID NO: 252 - protein, 21 aa, Homo sapiens
LILQGNRIRSVTKKAFTGLDA

SEQ ID NO: 253 - DNA, 63 bp, Homo sapiens
ttaattttacaaggtaaccgtattcgttctgttacaaaaaaagcattcacaggtttagatgca

SEQ ID NO: 254 - protein, 21 aa, Homo sapiens
MKYGADPSLINGEGCSCIHLA

SEQ ID NO: 255 - DNA, 63 bp, Homo sapiens
atgaaatatggtgcagatccatctttaattaacggtgaaggttgttcttgtattcatttagca

SEQ ID NO: 256 - protein, 21 aa, Homo sapiens
GFNENRQKRAVLAAQFEATSP

SEQ ID NO: 257 - DNA, 63 bp, Homo sapiens
ggtttcaacgaaaaccgtcaaaaacgtgcagttttagcagcacaattcgaagcaacatctcca

SEQ ID NO: 258 - protein, 21 aa, Homo sapiens
MPTINEDTPMRQRGSQSSGSD

SEQ ID NO: 259 - DNA, 63 bp, Homo sapiens
atgccaacaattaacgaagatacaccaatgcgtcaacgtggttctcaatcttctggttctgat

SEQ ID NO: 260 - protein, 21 aa, Homo sapiens
FVTRVVEEIKNMKIGDPLDRS

SEQ ID NO: 261 - DNA, 63 bp, Homo sapiens
ttcgttacacgtgttgttgaagaaattaaaaacatgaaaattggtgatccattagatcgttct

SEQ ID NO: 262 - protein, 21 aa, Homo sapiens
NLLLLGATAIQDRLQAGVPET

SEQ ID NO: 263 - DNA, 63 bp, Homo sapiens
aacttattattattaggtgcaacagcaattcaagatcgtttacaagcaggtgttccagaaaca

SEQ ID NO: 264 - protein, 21 aa, Homo sapiens
RSIQGPSLSSRRNVMSEASLD

SEQ ID NO: 265 - DNA, 63 bp, Homo sapiens
cgttctattcaaggtccatctttatcttctcgtcgtaacgttatgtctgaagcatctttagat

SEQ ID NO: 266 - protein, 21 aa, Homo sapiens
LNIPAPLHPEKTVNMTYQPTT

SEQ ID NO: 267 - DNA, 63 bp, Homo sapiens
ttaaacattccagcaccattacatccagaaaaaacagttaacatgacatatcaaccaacaaca

SEQ ID NO: 268 - protein, 21 aa, Homo sapiens
LKNGRLTVGQNIILHGAELVG

SEQ ID NO: 269 - DNA, 63 bp, Homo sapiens
ttaaaaaacggtcgtttaacagttggtcaaaacattattttacatggtgcagaattagttggt

SEQ ID NO: 270 - protein, 21 aa, Homo sapiens
HAEALLKAAIQYGTEEDVVKS

SEQ ID NO: 271 - DNA, 63 bp, Homo sapiens
catgcagaagcattattaaaagcagcaattcaatatggtacagaagaagatgttgttaaatct

SEQ ID NO: 272 - protein, 21 aa, Homo sapiens
YLKQWRSYNICIWDTAGREQF

SEQ ID NO: 273 - DNA, 63 bp, Homo sapiens
tatttaaaacaatggcgttcttataacatttgtatttgggatacagcaggtcgtgaacaattc

SEQ ID NO: 274 - protein, 21 aa, Homo sapiens
TGIVSWGQGCTTVGHFGVYTR

SEQ ID NO: 275 - DNA, 63 bp, Homo sapiens
acaggtattgtttcttggggtcaaggttgtacaacagttggtcatttcggtgtttatacacgt

SEQ ID NO: 276 - protein, 21 aa, Homo sapiens
KSRDAARSRRRKENFEFYELA

SEQ ID NO: 277 - DNA, 63 bp, Homo sapiens
aaatctcgtgatgcagcacgttctcgtcgtcgtaaagaaaacttcgaattctatgaattagca

SEQ ID NO: 278 - protein, 21 aa, Homo sapiens
MRGKPKVIDLKRNEATVETLT

SEQ ID NO: 279 - DNA, 63 bp, Homo sapiens
atgcgtggtaaaccaaaagttattgatttaaaacgtaacgaagcaacagttgaaacattaaca

SEQ ID NO: 280 - protein, 21 aa, Homo sapiens
ETHKVAAPDVIVPTLDTVRHE

SEQ ID NO: 281 - DNA, 63 bp, Homo sapiens
gaaacacataaagttgcagcaccagatgttattgttccaacattagatacagttcgtcatgaa

SEQ ID NO: 282 - protein, 21 aa, Homo sapiens
CLAEAAVAAVSVADTVRECPP

SEQ ID NO: 283 - DNA, 63 bp, Homo sapiens
tgtttagcagaagcagcagttgcagcagtttctgttgcagatacagttcgtgaatgtccacca

SEQ ID NO: 284 - protein, 21 aa, Homo sapiens
AILLLLQLWDCGAQETDNERS

SEQ ID NO: 285 - DNA, 63 bp, Homo sapiens
gcaattttattattattacaattatgggattgtggtgcacaagaaacagataacgaacgttct

SEQ ID NO: 286 - protein, 21 aa, Homo sapiens
YVRAFLKHSAKLVYMLESKHL

SEQ ID NO: 287 - DNA, 63 bp, Homo sapiens
tatgttcgtgcattcttaaaacattctgcaaaattagtttatatgttagaatctaaacattta

SEQ ID NO: 288 - protein, 21 aa, Homo sapiens
VRGQCDRRRHDTAATEIGGKI

SEQ ID NO: 289 - DNA, 63 bp, Homo sapiens
gttcgtggtcaatgtgatcgtcgtcgtcatgatacagcagcaacagaaattggtggtaaaatt

SEQ ID NO: 290 - protein, 21 aa, Homo sapiens
FQHIADYLPSNARPRFLRIQD

SEQ ID NO: 291 - DNA, 63 bp, Homo sapiens
ttccaacatattgcagattatttaccatctaacgcacgtccacgtttcttacgtattcaagat

SEQ ID NO: 292 - protein, 21 aa, Homo sapiens
SNEVGEGPFSSSVELAVLPKE

SEQ ID NO: 293 - DNA, 63 bp, Homo sapiens
tctaacgaagttggtgaaggtccattctcttcttctgttgaattagcagttttaccaaaagaa

SEQ ID NO: 294 - protein, 21 aa, Homo sapiens
VKADPAALMALLHLLPSPTPN

SEQ ID NO: 295 - DNA, 63 bp, Homo sapiens
gttaaagcagatccagcagcattaatggcattattacatttattaccatctccaacaccaaac

SEQ ID NO: 296 - protein, 21 aa, Homo sapiens
IEVEKMRRAETSARVENSPQV

SEQ ID NO: 297 - DNA, 63 bp, Homo sapiens
attgaagttgaaaaaatgcgtcgtgcagaaacatctgcacgtgttgaaaactctccacaagtt

SEQ ID NO: 298 - protein, 21 aa, Homo sapiens
TAYRFLEEMRWRLPLANMSYY

SEQ ID NO: 299 - DNA, 63 bp, Homo sapiens
acagcatatcgtttcttagaagaaatgcgttggcgtttaccattagcaaacatgtcttattat

SEQ ID NO: 300 - protein, 21 aa, Homo sapiens
VPGDLSQTSEEQSLSDFEISN

SEQ ID NO: 301 - DNA, 63 bp, Homo sapiens
gttccaggtgatttatctcaaacatctgaagaacaatctttatctgatttcgaaatttctaac

SEQ ID NO: 302 - protein, 21 aa, Homo sapiens
FSDLDNMASRGHDHLDNHRES

SEQ ID NO: 303 - DNA, 63 bp, Homo sapiens
ttctctgatttagataacatggcatctcgtggtcatgatcatttagataaccatcgtgaatct

SEQ ID NO: 304 - protein, 21 aa, Homo sapiens
GPAHCLLLGEWVALNTLARCS

SEQ ID NO: 305 - DNA, 63 bp, Homo sapiens
ggtccagcacattgtttattattaggtgaatgggttgcattaaacacattagcacgttgttct

SEQ ID NO: 306 - protein, 21 aa, Homo sapiens
SRFKGKHSLDGDEEEDDDDGG

SEQ ID NO: 307 - DNA, 63 bp, Homo sapiens
tctcgtttcaaaggtaaacattctttagatggtgatgaagaagaagatgatgatgatggtggt

SEQ ID NO: 308 - protein, 21 aa, Homo sapiens
AENTRLCTKAPGPSRTESTQR

SEQ ID NO: 309 - DNA, 63 bp, Homo sapiens
gcagaaaacacacgtttatgtacaaaagcaccaggtccatctcgtacagaatctacacaacgt

SEQ ID NO: 310 - protein, 21 aa, Homo sapiens
VFCEMRPVLECEEELYSSCRQ

SEQ ID NO: 311 - DNA, 63 bp, Homo sapiens
gttttctgtgaaatgcgtccagttttagaatgtgaagaagaattatattcttcttgtcgtcaa

SEQ ID NO: 312 - protein, 21 aa, Homo sapiens
PLPAQAQSAWAPAPAPATYRA

SEQ ID NO: 313 - DNA, 63 bp, Homo sapiens
ccattaccagcacaagcacaatctgcatgggcaccagcaccagcaccagcaacatatcgtgca

SEQ ID NO: 314 - protein, 21 aa, Homo sapiens
PFPADGLGAEDAFQCLEETPF

SEQ ID NO: 315 - DNA, 63 bp, Homo sapiens
ccattcccagcagatggtttaggtgcagaagatgcattccaatgtttagaagaaacaccattc

SEQ ID NO: 316 - protein, 21 aa, Homo sapiens
LTLNNDQVFPINPPKFDKIED

SEQ ID NO: 317 - DNA, 63 bp, Homo sapiens
ttaacattaaacaacgatcaagttttcccaattaacccaccaaaattcgataaaattgaagat

SEQ ID NO: 318 - protein, 21 aa, Homo sapiens
VHARACGRFFHKGQGTCFYFG

SEQ ID NO: 319 - DNA, 63 bp, Homo sapiens
gttcatgcacgtgcatgtggtcgtttcttccataaaggtcaaggtacatgtttctatttcggt

SEQ ID NO: 320 - protein, 21 aa, Homo sapiens
HPAPPHHTGTEWQPVDPGRAA

SEQ ID NO: 321 - DNA, 63 bp, Homo sapiens
catccagcaccaccacatcatacaggtacagaatggcaaccagttgatccaggtcgtgcagca

SEQ ID NO: 322 - protein, 21 aa, Homo sapiens
EQRRTVRIYFVGPSAVLPEVE

SEQ ID NO: 323 - DNA, 63 bp, Homo sapiens
gaacaacgtcgtacagttcgtatttatttcgttggtccatctgcagttttaccagaagttgaa

SEQ ID NO: 324 - protein, 21 aa, Homo sapiens
PPPPPLPAAPQAPEFPWMKEK

SEQ ID NO: 325 - DNA, 63 bp, Homo sapiens
ccaccaccaccaccattaccagcagcaccacaagcaccagaattcccatggatgaaagaaaaa

SEQ ID NO: 326 - protein, 21 aa, Homo sapiens
FRYGHTLIQPLMFRLDNRYQP

SEQ ID NO: 327 - DNA, 63 bp, Homo sapiens
ttccgttatggtcatacattaattcaaccattaatgttccgtttagataaccgttatcaacca

SEQ ID NO: 328 - protein, 21 aa, Homo sapiens
IRNAVEMMIAWLDTQLKNKLI

SEQ ID NO: 329 - DNA, 63 bp, Homo sapiens
attcgtaacgcagttgaaatgatgattgcatggttagatacacaattaaaaaacaaattaatt

SEQ ID NO: 330 - protein, 21 aa, Homo sapiens
MSSTTTLTTPHNQVDMLLQEM

SEQ ID NO: 331 - DNA, 63 bp, Homo sapiens
atgtcttctacaacaacattaacaacaccacataaccaagttgatatgttattacaagaaatg

SEQ ID NO: 332 - protein, 21 aa, Homo sapiens
TLVDSRTSVTNVKFAPKHMGL

SEQ ID NO: 333 - DNA, 63 bp, Homo sapiens
acattagttgattctcgtacatctgttacaaacgttaaattcgcaccaaaacatatgggttta

SEQ ID NO: 334 - protein, 21 aa, Homo sapiens
WKQDRETGRFLRPCECLVVRV

SEQ ID NO: 335 - DNA, 63 bp, Homo sapiens
tggaaacaagatcgtgaaacaggtcgtttcttacgtccatgtgaatgtttagttgttcgtgtt

SEQ ID NO: 336 - protein, 21 aa, Homo sapiens
LLKGPPPFEDSLAFDGLSLAR

SEQ ID NO: 337 - DNA, 63 bp, Homo sapiens
ttattaaaaggtccaccaccattcgaagattctttagcattcgatggtttatctttagcacgt

SEQ ID NO: 338 - protein, 21 aa, Homo sapiens
YMHFGEVAKQKEFFNLSHCQL

SEQ ID NO: 339 - DNA, 63 bp, Homo sapiens
tatatgcatttcggtgaagttgcaaaacaaaaagaattcttcaacttatctcattgtcaatta

SEQ ID NO: 340 - protein, 21 aa, Homo sapiens
LAVELQAGAGEPGLAALVAFV

SEQ ID NO: 341 - DNA, 63 bp, Homo sapiens
ttagcagttgaattacaagcaggtgcaggtgaaccaggtttagcagcattagttgcattcgtt

SEQ ID NO: 342 - protein, 21 aa, Homo sapiens
FKWSSALTTLNQHKRIHTGEK

SEQ ID NO: 343 - DNA, 63 bp, Homo sapiens
ttcaaatggtcttctgcattaacaacattaaaccaacataaacgtattcatacaggtgaaaaa

SEQ ID NO: 344 - protein, 21 aa, Homo sapiens
RFAPPNMQAAQAVSHIQSSGP

SEQ ID NO: 345 - DNA, 63 bp, Homo sapiens
cgtttcgcaccaccaaacatgcaagcagcacaagcagtttctcatattcaatcttctggtcca

SEQ ID NO: 346 - protein, 21 aa, Homo sapiens
VRQVPGFGVAHALGNRVGEAA

SEQ ID NO: 347 - DNA, 63 bp, Homo sapiens
gttcgtcaagttccaggtttcggtgttgcacatgcattaggtaaccgtgttggtgaagcagca

SEQ ID NO: 348 - protein, 21 aa, Homo sapiens
ANRVVICYFGKGAASEGDAHA

SEQ ID NO: 349 - DNA, 63 bp, Homo sapiens
gcaaaccgtgttgttatttgttatttcggtaaaggtgcagcatctgaaggtgatgcacatgca

SEQ ID NO: 350 - protein, 21 aa, Homo sapiens
RRDTLRLMCSRPSPTAEVRWF

SEQ ID NO: 351 - DNA, 63 bp, Homo sapiens
cgtcgtgatacattacgtttaatgtgttctcgtccatctccaacagcagaagttcgttggttc

SEQ ID NO: 352 - protein, 21 aa, Homo sapiens
TVPKEPASAPEAPTLPTTAPA

SEQ ID NO: 353 - DNA, 63 bp, Homo sapiens
acagttccaaaagaaccagcatctgcaccagaagcaccaacattaccaacaacagcaccagca

SEQ ID NO: 354 - protein, 21 aa, Homo sapiens
VKAQTRAALLLVLQEDEEHWG

SEQ ID NO: 355 - DNA, 63 bp, Homo sapiens
gttaaagcacaaacacgtgcagcattattattagttttacaagaagatgaagaacattggggt

SEQ ID NO: 356 - protein, 21 aa, Homo sapiens
LSQNPPSQIFHSLSGNSRPLQ

SEQ ID NO: 357 - DNA, 63 bp, Homo sapiens
ttatctcaaaacccaccatctcaaattttccattctttatctggtaactctcgtccattacaa

SEQ ID NO: 358 - protein, 21 aa, Homo sapiens
VESKAVKLLCSVLRSPRCRLQ

SEQ ID NO: 359 - DNA, 63 bp, Homo sapiens
gttgaatctaaagcagttaaattattatgttctgttttacgttctccacgttgtcgtttacaa

SEQ ID NO: 360 - protein, 21 aa, Homo sapiens
PYECNICEKALSHRGSLTLHQ

SEQ ID NO: 361 - DNA, 63 bp, Homo sapiens
ccatatgaatgtaacatttgtgaaaaagcattatctcatcgtggttctttaacattacatcaa

SEQ ID NO: 362 - protein, 21 aa, Homo sapiens
MSVGNIGNYLTTSPASLNPEE

SEQ ID NO: 363 - DNA, 63 bp, Homo sapiens
atgtctgttggtaacattggtaactatttaacaacatctccagcatctttaaacccagaagaa

SEQ ID NO: 364 - protein, 21 aa, Homo sapiens
VLVSLDSVEPPDWSFGEEEDG

SEQ ID NO: 365 - DNA, 63 bp, Homo sapiens
gttttagtttctttagattctgttgaaccaccagattggtctttcggtgaagaagaagatggt

SEQ ID NO: 366 - protein, 21 aa, Homo sapiens
PEPTSGVIAREAEIMETSIQA

SEQ ID NO: 367 - DNA, 63 bp, Homo sapiens
ccagaaccaacatctggtgttattgcacgtgaagcagaaattatggaaacatctattcaagca

SEQ ID NO: 368 - protein, 21 aa, Homo sapiens
GVCGEAFCENLEGSFLCVCAD

SEQ ID NO: 369 - DNA, 63 bp, Homo sapiens
ggtgtttgtggtgaagcattctgtgaaaacttagaaggttctttcttatgtgtttgtgcagat

SEQ ID NO: 370 - protein, 21 aa, Homo sapiens
EPDRNALRRKQRERRNQETQQ

SEQ ID NO: 371 - DNA, 63 bp, Homo sapiens
gaaccagatcgtaacgcattacgtcgtaaacaacgtgaacgtcgtaaccaagaaacacaacaa

SEQ ID NO: 372 - protein, 21 aa, Homo sapiens
QEYLAAKGKLNSQNTKPYLKS

SEQ ID NO: 373 - DNA, 63 bp, Homo sapiens
caagaatatttagcagcaaaaggtaaattaaactctcaaaacacaaaaccatatttaaaatct

SEQ ID NO: 374 - protein, 21 aa, Homo sapiens
KQPPHQVEVEVAEQNVSRICH

SEQ ID NO: 375 - DNA, 63 bp, Homo sapiens
aaacaaccaccacatcaagttgaagttgaagttgcagaacaaaacgtttctcgtatttgtcat

SEQ ID NO: 376 - protein, 21 aa, Homo sapiens
SLRNLYTPNVMVSRLLILGGA

SEQ ID NO: 377 - DNA, 63 bp, Homo sapiens
tctttacgtaacttatatacaccaaacgttatggtttctcgtttattaattttaggtggtgca

SEQ ID NO: 378 - protein, 21 aa, Homo sapiens
DLHFMKKIPPVAEASNILVGE

SEQ ID NO: 379 - DNA, 63 bp, Homo sapiens
gatttacatttcatgaaaaaaattccaccagttgcagaagcatctaacattttagttggtgaa

SEQ ID NO: 380 - protein, 21 aa, Homo sapiens
NGVVSLVGGPCTLTSAGQLLP

SEQ ID NO: 381 - DNA, 63 bp, Homo sapiens
aacggtgttgtttctttagttggtggtccatgtacattaacatctgcaggtcaattattacca

SEQ ID NO: 382 - protein, 21 aa, Homo sapiens
SILGEEPRFGKTPLAMLAATC

SEQ ID NO: 383 - DNA, 63 bp, Homo sapiens
tctattttaggtgaagaaccacgtttcggtaaaacaccattagcaatgttagcagcaacatgt

SEQ ID NO: 384 - protein, 21 aa, Homo sapiens
DEYLISQSERYSITMTEKSTI

SEQ ID NO: 385 - DNA, 63 bp, Homo sapiens
gatgaatatttaatttctcaatctgaacgttattctattacaatgacagaaaaatctacaatt

SEQ ID NO: 386 - protein, 21 aa, Homo sapiens
LCDLSDLEYLHEEFHQSLQWM

SEQ ID NO: 387 - DNA, 63 bp, Homo sapiens
ttatgtgatttatctgatttagaatatttacatgaagaattccatcaatctttacaatggatg

SEQ ID NO: 388 - protein, 21 aa, Homo sapiens
ESFRAAIDKSCDGPEEIEADG

SEQ ID NO: 389 - DNA, 63 bp, Homo sapiens
gaatctttccgtgcagcaattgataaatcttgtgatggtccagaagaaattgaagcagatggt

SEQ ID NO: 390 - protein, 21 aa, Homo sapiens
TLIQGVLRINSKKFHEAFIPS

SEQ ID NO: 391 - DNA, 63 bp, Homo sapiens
acattaattcaaggtgttttacgtattaactctaaaaaattccatgaagcattcattccatct

SEQ ID NO: 392 - protein, 21 aa, Homo sapiens
KQASLELGEGKLPAACLKPAL

SEQ ID NO: 393 - DNA, 63 bp, Homo sapiens
aaacaagcatctttagaattaggtgaaggtaaattaccagcagcatgtttaaaaccagcatta

SEQ ID NO: 394 - protein, 21 aa, Homo sapiens
DSEGPAEGEGHLGRCMRRLRD

SEQ ID NO: 395 - DNA, 63 bp, Homo sapiens
gattctgaaggtccagcagaaggtgaaggtcatttaggtcgttgtatgcgtcgtttacgtgat

SEQ ID NO: 396 - protein, 21 aa, Homo sapiens
AVRRLRYKGGDTFTGLALTHV

SEQ ID NO: 397 - DNA, 63 bp, Homo sapiens
gcagttcgtcgtttacgttataaaggtggtgatacattcacaggtttagcattaacacatgtt

SEQ ID NO: 398 - protein, 21 aa, Homo sapiens
VRTCMHNQKDVVQMPSETLKA

SEQ ID NO: 399 - DNA, 63 bp, Homo sapiens
gttcgtacatgtatgcataaccaaaaagatgttgttcaaatgccatctgaaacattaaaagca

SEQ ID NO: 400 - protein, 21 aa, Homo sapiens
DNMPLELSGRLQKTVTGLLIE

SEQ ID NO: 401 - DNA, 63 bp, Homo sapiens
gataacatgccattagaattatctggtcgtttacaaaaaacagttacaggtttattaattgaa

SEQ ID NO: 402 - protein, 21 aa, Homo sapiens
QSSCQPACCTTSSCQQACCVP

SEQ ID NO: 403 - DNA, 63 bp, Homo sapiens
caatcttcttgtcaaccagcatgttgtacaacatcttcttgtcaacaagcatgttgtgttcca

SEQ ID NO: 404 - protein, 21 aa, Homo sapiens
IKVKSEEKIKAVEKSEKETVI

SEQ ID NO: 405 - DNA, 63 bp, Homo sapiens
attaaagttaaatctgaagaaaaaattaaagcagttgaaaaatctgaaaaagaaacagttatt

SEQ ID NO: 406 - protein, 21 aa, Homo sapiens
VRRTKLQMQTAALEFRQRIIL

SEQ ID NO: 407 - DNA, 63 bp, Homo sapiens
gttcgtcgtacaaaattacaaatgcaaacagcagcattagaattccgtcaacgtattatttta

SEQ ID NO: 408 - protein, 21 aa, Homo sapiens
DLYMDTGEEICFRVVDESFVD

SEQ ID NO: 409 - DNA, 63 bp, Homo sapiens
gatttatatatggatacaggtgaagaaatttgtttccgtgttgttgatgaatctttcgttgat

SEQ ID NO: 410 - protein, 21 aa, Homo sapiens
ALNTSTCPPNRVKLDIFPKEC

SEQ ID NO: 411 - DNA, 63 bp, Homo sapiens
gcattaaacacatctacatgtccaccaaaccgtgttaaattagatattttcccaaaagaatgt

SEQ ID NO: 412 - protein, 21 aa, Homo sapiens
EYVKWFLHADLSSYLTPMKHM

SEQ ID NO: 413 - DNA, 63 bp, Homo sapiens
gaatatgttaaatggttcttacatgcagatttatcttcttatttaacaccaatgaaacatatg

SEQ ID NO: 414 - protein, 21 aa, Homo sapiens
ISRDAAQKIYLEMKFTLRDCN

SEQ ID NO: 415 - DNA, 63 bp, Homo sapiens
atttctcgtgatgcagcacaaaaaatttatttagaaatgaaattcacattacgtgattgtaac

SEQ ID NO: 416 - protein, 21 aa, Homo sapiens
NCSFEDFTESKSVLEDGRIHC

SEQ ID NO: 417 - DNA, 63 bp, Homo sapiens
aactgttctttcgaagatttcacagaatctaaatctgttttagaagatggtcgtattcattgt

SEQ ID NO: 418 - protein, 21 aa, Homo sapiens
SKKFLENLLEIFNSHVDHGTG

SEQ ID NO: 419 - DNA, 63 bp, Homo sapiens
tctaaaaaattcttagaaaacttattagaaattttcaactctcatgttgatcatggtacaggt

SEQ ID NO: 420 - protein, 21 aa, Homo sapiens
SSYVKLSVGKNTHTSKTCPHN

SEQ ID NO: 421 - DNA, 63 bp, Homo sapiens
tcttcttatgttaaattatctgttggtaaaaacacacatacatctaaaacatgtccacataac

SEQ ID NO: 422 - protein, 21 aa, Homo sapiens
HRGVGSQTASGRSGQVLGVVL

SEQ ID NO: 423 - DNA, 63 bp, Homo sapiens
catcgtggtgttggttctcaaacagcatctggtcgttctggtcaagttttaggtgttgtttta

SEQ ID NO: 424 - protein, 21 aa, Homo sapiens
LTQDIIMKILQKKSVEVNFTE

SEQ ID NO: 425 - DNA, 63 bp, Homo sapiens
ttaacacaagatattattatgaaaattttacaaaaaaaatctgttgaagttaacttcacagaa

SEQ ID NO: 426 - protein, 21 aa, Homo sapiens
IYIPDDIPIPTEFELRESNMP

SEQ ID NO: 427 - DNA, 63 bp, Homo sapiens
atttatattccagatgatattccaattccaacagaattcgaattacgtgaatctaacatgcca

SEQ ID NO: 428 - protein, 21 aa, Homo sapiens
RPLLGDRQYIKKFYTDAAFLL

SEQ ID NO: 429 - DNA, 63 bp, Homo sapiens
cgtccattattaggtgatcgtcaatatattaaaaaattctatacagatgcagcattcttatta

SEQ ID NO: 430 - protein, 21 aa, Homo sapiens
PSEGVQAAGGLAQPLGVPPGS

SEQ ID NO: 431 - DNA, 63 bp, Homo sapiens
ccatctgaaggtgttcaagcagcaggtggtttagcacaaccattaggtgttccaccaggttct

SEQ ID NO: 432 - protein, 21 aa, Homo sapiens
SLDSFGSRSRHTPSPDVVLRG

SEQ ID NO: 433 - DNA, 63 bp, Homo sapiens
tctttagattctttcggttctcgttctcgtcatacaccatctccagatgttgttttacgtggt

SEQ ID NO: 434 - protein, 21 aa, Homo sapiens
PLPTTVGDFWHMIWEQKSTVI

SEQ ID NO: 435 - DNA, 63 bp, Homo sapiens
ccattaccaacaacagttggtgatttctggcatatgatttgggaacaaaaatctacagttatt

SEQ ID NO: 436 - protein, 21 aa, Homo sapiens
GPLGAGIALAYKYKGNDEICL

SEQ ID NO: 437 - DNA, 63 bp, Homo sapiens
ggtccattaggtgcaggtattgcattagcatataaatataaaggtaacgatgaaatttgttta

SEQ ID NO: 438 - protein, 21 aa, Homo sapiens
VKVAEITELIFKALENDQKVR

SEQ ID NO: 439 - DNA, 63 bp, Homo sapiens
gttaaagttgcagaaattacagaattaattttcaaagcattagaaaacgatcaaaaagttcgt

SEQ ID NO: 440 - protein, 21 aa, Homo sapiens
LEELYALFKAQHLTSCYWGGS

SEQ ID NO: 441 - DNA, 63 bp, Homo sapiens
ttagaagaattatatgcattattcaaagcacaacatttaacatcttgttattggggtggttct

SEQ ID NO: 442 - protein, 21 aa, Homo sapiens
STADYAMFKVEPEADKYRLTY

SEQ ID NO: 443 - DNA, 63 bp, Homo sapiens
tctacagcagattatgcaatgttcaaagttgaaccagaagcagataaatatcgtttaacatat

SEQ ID NO: 444 - protein, 21 aa, Homo sapiens
VDSFLQADGPQERPPAALPES

SEQ ID NO: 445 - DNA, 63 bp, Homo sapiens
gttgattctttcttacaagcagatggtccacaagaacgtccaccagcagcattaccagaatct

SEQ ID NO: 446 - protein, 21 aa, Homo sapiens
YPEIVKENKLLEHYYQELKIV

SEQ ID NO: 447 - DNA, 63 bp, Homo sapiens
tatccagaaattgttaaagaaaacaaattattagaacattattatcaagaattaaaaattgtt

SEQ ID NO: 448 - protein, 21 aa, Homo sapiens
NELYLSQEDFSINGNIKVFPD

SEQ ID NO: 449 - DNA, 63 bp, Homo sapiens
aacgaattatatttatctcaagaagatttctctattaacggtaacattaaagttttcccagat

SEQ ID NO: 450 - protein, 21 aa, Homo sapiens
GLPEAKIFSGTSSEQFGYAVQ

SEQ ID NO: 451 - DNA, 63 bp, Homo sapiens
ggtttaccagaagcaaaaattttctctggtacatcttctgaacaattcggttatgcagttcaa

SEQ ID NO: 452 - protein, 21 aa, Homo sapiens
YKPVQHVTALSTVSSCHKMVS

SEQ ID NO: 453 - DNA, 63 bp, Homo sapiens
tataaaccagttcaacatgttacagcattatctacagtttcttcttgtcataaaatggtttct

SEQ ID NO: 454 - protein, 21 aa, Homo sapiens
LLLRLPSLRTMSSSVIEQLFF

SEQ ID NO: 455 - DNA, 63 bp, Homo sapiens
ttattattacgtttaccatctttacgtacaatgtcttcttctgttattgaacaattattcttc

SEQ ID NO: 456 - protein, 21 aa, Homo sapiens
KGACWIYDNINMAHMLVAISV

SEQ ID NO: 457 - DNA, 63 bp, Homo sapiens
aaaggtgcatgttggatttatgataacattaacatggcacatatgttagttgcaatttctgtt

SEQ ID NO: 458 - protein, 21 aa, Homo sapiens
CGSSEGTIYLLNWNGFGATSD

SEQ ID NO: 459 - DNA, 63 bp, Homo sapiens
tgtggttcttctgaaggtacaatttatttattaaactggaacggtttcggtgcaacatctgat

SEQ ID NO: 460 - protein, 21 aa, Homo sapiens
KDHGEPALTASATVLVSLVES

SEQ ID NO: 461 - DNA, 63 bp, Homo sapiens
aaagatcatggtgaaccagcattaacagcatctgcaacagttttagtttctttagttgaatct

SEQ ID NO: 462 - protein, 21 aa, Homo sapiens
RSAEPGYLVTMVVAVDRDSGQ

SEQ ID NO: 463 - DNA, 63 bp, Homo sapiens
cgttctgcagaaccaggttatttagttacaatggttgttgcagttgatcgtgattctggtcaa

SEQ ID NO: 464 - protein, 21 aa, Homo sapiens
LFYLFASLTSHLPWEHCGNWW

SEQ ID NO: 465 - DNA, 63 bp, Homo sapiens
ttattctatttattcgcatctttaacatctcatttaccatgggaacattgtggtaactggtgg

SEQ ID NO: 466 - protein, 21 aa, Homo sapiens
SDGEVPTAVASAQEKSLGNIL

SEQ ID NO: 467 - DNA, 63 bp, Homo sapiens
tctgatggtgaagttccaacagcagttgcatctgcacaagaaaaatctttaggtaacatttta

SEQ ID NO: 468 - protein, 21 aa, Homo sapiens
YESPNEEEEAHVEDDADYEPP

SEQ ID NO: 469 - DNA, 63 bp, Homo sapiens
tatgaatctccaaacgaagaagaagaagcacatgttgaagatgatgcagattatgaaccacca

SEQ ID NO: 470 - protein, 21 aa, Homo sapiens
KLGFVKFAQTMFKLITGTLSK

SEQ ID NO: 471 - DNA, 63 bp, Homo sapiens
aaattaggtttcgttaaattcgcacaaacaatgttcaaattaattacaggtacattatctaaa

SEQ ID NO: 472 - protein, 21 aa, Homo sapiens
GLEKTASISISDPFFRSAQRW

SEQ ID NO: 473 - DNA, 63 bp, Homo sapiens
ggtttagaaaaaacagcatctatttctatttctgatccattcttccgttctgcacaacgttgg

SEQ ID NO: 474 - protein, 21 aa, Homo sapiens
VAPPQGVVKASYSLSEDAEGE

SEQ ID NO: 475 - DNA, 63 bp, Homo sapiens
gttgcaccaccacaaggtgttgttaaagcatcttattctttatctgaagatgcagaaggtgaa

SEQ ID NO: 476 - protein, 21 aa, Homo sapiens
QQRLQEELDHKLGPGASSSRV

SEQ ID NO: 477 - DNA, 63 bp, Homo sapiens
caacaacgtttacaagaagaattagatcataaattaggtccaggtgcatcttcttctcgtgtt

SEQ ID NO: 478 - protein, 21 aa, Homo sapiens
PPGLLVLLWQVTTLPEHCFSW

SEQ ID NO: 479 - DNA, 63 bp, Homo sapiens
ccaccaggtttattagttttattatggcaagttacaacattaccagaacattgtttctcttgg

SEQ ID NO: 480 - protein, 21 aa, Homo sapiens
RQTSRSECRDFVVPGSKPSGN

SEQ ID NO: 481 - DNA, 63 bp, Homo sapiens
cgtcaaacatctcgttctgaatgtcgtgatttcgttgttccaggttctaaaccatctggtaac

SEQ ID NO: 482 - protein, 21 aa, Homo sapiens
DYAFIHFDERHGAVKAMEEMN

SEQ ID NO: 483 - DNA, 63 bp, Homo sapiens
gattatgcattcattcatttcgatgaacgtcatggtgcagttaaagcaatggaagaaatgaac

SEQ ID NO: 484 - protein, 21 aa, Homo sapiens
RKGNASKEQVGLHDLVQQLPK

SEQ ID NO: 485 - protein, 63 aa, Homo sapiens
CGTAAAGGTAACGCATCTAAAGAACAAGTTGGTTTACATGATTTAGTTCAACAATTACCAAAA

SEQ ID NO: 486 - protein, 21 aa, Homo sapiens
TLPPNSPNGYCGSNINYPESH

SEQ ID NO: 487 - DNA, 63 bp, Homo sapiens
acattaccaccaaactctccaaacggttattgtggttctaacattaactatccagaatctcat

SEQ ID NO: 488 - protein, 21 aa, Homo sapiens
EAMTEKLQYLASVYCTEKMSQ

SEQ ID NO: 489 - DNA, 63 bp, Homo sapiens
gaagcaatgacagaaaaattacaatatttagcatctgtttattgtacagaaaaaatgtctcaa

SEQ ID NO: 490 - protein, 21 aa, Homo sapiens
VQGRVQEELDHVLGPGRTPRL

SEQ ID NO: 491 - DNA, 63 bp, Homo sapiens
gttcaaggtcgtgttcaagaagaattagatcatgttttaggtccaggtcgtacaccacgttta

SEQ ID NO: 492 - protein, 21 aa, Homo sapiens
SERKKWIHCFQGVTCIIFCAA

SEQ ID NO: 493 - DNA, 63 bp, Homo sapiens
tctgaacgtaaaaaatggattcattgtttccaaggtgttacatgtattattttctgtgcagca

SEQ ID NO: 494 - protein, 21 aa, Homo sapiens
GSGAFSPVCTCLNRGRRSEDQ

SEQ ID NO: 495 - DNA, 63 bp, Homo sapiens
ggttctggtgcattctctccagtttgtacatgtttaaaccgtggtcgtcgttctgaagatcaa

SEQ ID NO: 496 - protein, 21 aa, Homo sapiens
NQLKEISSSTDSQKKEETKGK

SEQ ID NO: 497 - DNA, 63 bp, Homo sapiens
aaccaattaaaagaaatttcttcttctacagattctcaaaaaaaagaagaaacaaaaggtaaa

SEQ ID NO: 498 - protein, 21 aa, Homo sapiens
TQSSKRLLPSVGLSTVLLKKE

SEQ ID NO: 499 - DNA, 63 bp, Homo sapiens
acacaatcttctaaacgtttattaccatctgttggtttatctacagttttattaaaaaaagaa

SEQ ID NO: 500 - protein, 21 aa, Homo sapiens
LGARGSGAAAGTVRELLQDGC

SEQ ID NO: 501 - DNA, 63 bp, Homo sapiens
ttaggtgcacgtggttctggtgcagcagcaggtacagttcgtgaattattacaagatggttgt

SEQ ID NO: 502 - protein, 21 aa, Homo sapiens
PPPPRLQGPPFQTPLPGPHHP

SEQ ID NO: 503 - DNA, 63 bp, Homo sapiens
ccaccaccaccacgtttacaaggtccaccattccaaacaccattaccaggtccacatcatcca

SEQ ID NO: 504 - protein, 21 aa, Homo sapiens
MQQILQQQVLCPQQLQALLQQ

SEQ ID NO: 505 - DNA, 63 bp, Homo sapiens
atgcaacaaattttacaacaacaagttttatgtccacaacaattacaagcattattacaacaa

SEQ ID NO: 506 - protein, 21 aa, Homo sapiens
GELIRTQPQRFPQLQTSAQVP

SEQ ID NO: 507 - DNA, 63 bp, Homo sapiens
ggtgaattaattcgtacacaaccacaacgtttcccacaattacaaacatctgcacaagttcca

SEQ ID NO: 508 - protein, 21 aa, Homo sapiens
AKMRHGFSGILITETGTMGSK

SEQ ID NO: 509 - DNA, 63 bp, Homo sapiens
gcaaaaatgcgtcatggtttctctggtattttaattacagaaacaggtacaatgggttctaaa

SEQ ID NO: 510 - protein, 21 aa, Homo sapiens
SLLLNSSQSSSSEETVLRSDA

SEQ ID NO: 511 - DNA, 63 bp, Homo sapiens
tctttattattaaactcttctcaatcttcttcttctgaagaaacagttttacgttctgatgca

SEQ ID NO: 512 - protein, 21 aa, Homo sapiens
GVDQQDGKNHSGSPGCEESDA

SEQ ID NO: 513 - DNA, 63 bp, Homo sapiens
ggtgttgatcaacaagatggtaaaaaccattctggttctccaggttgtgaagaatctgatgca

SEQ ID NO: 514 - protein, 21 aa, Homo sapiens
PRPDILDMPSQHVAFPSSPQI

SEQ ID NO: 515 - DNA, 63 bp, Homo sapiens
ccacgtccagatattttagatatgccatctcaacatgttgcattcccatcttctccacaaatt

SEQ ID NO: 516 - protein, 21 aa, Homo sapiens
MLIPFCEELNFKTTWLFHKTR

SEQ ID NO: 517 - DNA, 63 bp, Homo sapiens
atgttaattccattctgtgaagaattaaacttcaaaacaacatggttattccataaaacacgt

SEQ ID NO: 518 - protein, 21 aa, Homo sapiens
ERMLFHGSPFFNAIIHKGFDE

SEQ ID NO: 519 - DNA, 63 bp, Homo sapiens
gaacgtatgttattccatggttctccattcttcaacgcaattattcataaaggtttcgatgaa

SEQ ID NO: 520 - protein, 21 aa, Homo sapiens
KGKKVKAQSTEPVVQDADWLA

SEQ ID NO: 521 - DNA, 63 bp, Homo sapiens
aaaggtaaaaaagttaaagcacaatctacagaaccagttgttcaagatgcagattggttagca

SEQ ID NO: 522 - protein, 21 aa, Homo sapiens
FICRKCQMMFADEDAAVNHQK

SEQ ID NO: 523 - DNA, 63 bp, Homo sapiens
ttcatttgtcgtaaatgtcaaatgatgttcgcagatgaagatgcagcagttaaccatcaaaaa

SEQ ID NO: 524 - protein, 21 aa, Homo sapiens
LYLLWLLLVTHAYNWNCCFIP

SEQ ID NO: 525 - DNA, 63 bp, Homo sapiens
ttatatttattatggttattattagttacacatgcatataactggaactgttgtttcattcca

SEQ ID NO: 526 - protein, 21 aa, Homo sapiens
RLTYPGIKVVVVIDGNSEDDL

SEQ ID NO: 527 - DNA, 63 bp, Homo sapiens
cgtttaacatatccaggtattaaagttgttgttgttattgatggtaactctgaagatgattta

SEQ ID NO: 528 - protein, 21 aa, Homo sapiens
PEPKIIPSEENPTFEDNPEVP

SEQ ID NO: 529 - DNA, 63 bp, Homo sapiens
ccagaaccaaaaattattccatctgaagaaaacccaacattcgaagataacccagaagttcca

SEQ ID NO: 530 - protein, 21 aa, Homo sapiens
EIERHGEKVEDCQRFAKQYIN

SEQ ID NO: 531 - DNA, 63 bp, Homo sapiens
gaaattgaacgtcatggtgaaaaagttgaagattgtcaacgtttcgcaaaacaatatattaac

SEQ ID NO: 532 - protein, 21 aa, Homo sapiens
FSSAVLISPDGAWLIEPRKVQ

SEQ ID NO: 533 - protein, 63 aa, Homo sapiens
TTCTCTTCTGCAGTTTTAATTTCTCCAGATGGTGCATGGTTAATTGAACCACGTAAAGTTCAA

SEQ ID NO: 534 - protein, 21 aa, Homo sapiens
ILEENMSLCHERHASKVNQQR

SEQ ID NO: 535 - DNA, 63 bp, Homo sapiens
attttagaagaaaacatgtctttatgtcatgaacgtcatgcatctaaagttaaccaacaacgt

SEQ ID NO: 536 - protein, 21 aa, Homo sapiens
RVDYISTRRASQHKCALLLQE

SEQ ID NO: 537 - DNA, 63 bp, Homo sapiens
cgtgttgattatatttctacacgtcgtgcatctcaacataaatgtgcattattattacaagaa

SEQ ID NO: 538 - protein, 21 aa, Homo sapiens
LVKEQAFGGLSRALALDCEMV

SEQ ID NO: 539 - DNA, 63 bp, Homo sapiens
ttagttaaagaacaagcattcggtggtttatctcgtgcattagcattagattgtgaaatggtt

SEQ ID NO: 540 - protein, 21 aa, Homo sapiens
LLYTMLTGYTLFANGPDDTPE

SEQ ID NO: 541 - DNA, 63 bp, Homo sapiens
ttattatatacaatgttaacaggttatacattattcgcaaacggtccagatgatacaccagaa

SEQ ID NO: 542 - protein, 21 aa, Homo sapiens
KVETEAKIKQQLREIEENQKR

SEQ ID NO: 543 - DNA, 63 bp, Homo sapiens
aaagttgaaacagaagcaaaaattaaacaacaattacgtgaaattgaagaaaaccaaaaacgt

SEQ ID NO: 544 - protein, 21 aa, Homo sapiens
YGVNYFSIKNNKGSELWLGVD

SEQ ID NO: 545 - DNA, 63 bp, Homo sapiens
tatggtgttaactatttctctattaaaaacaacaaaggttctgaattatggttaggtgttgat

SEQ ID NO: 546 - protein, 21 aa, Homo sapiens
DLTMEKITVECDHFRVLSYQA

SEQ ID NO: 547 - DNA, 63 bp, Homo sapiens
gatttaacaatggaaaaaattacagttgaatgtgatcatttccgtgttttatcttatcaagca

SEQ ID NO: 548 - protein, 21 aa, Homo sapiens
VEAAMSSKTLRKWNRLLQKGW

SEQ ID NO: 549 - DNA, 63 bp, Homo sapiens
gttgaagcagcaatgtcttctaaaacattacgtaaatggaaccgtttattacaaaaaggttgg

SEQ ID NO: 550 - protein, 21 aa, Homo sapiens
TGQWSEWSQPECFQAPQRQGP

SEQ ID NO: 551 - DNA, 63 bp, Homo sapiens
acaggtcaatggtctgaatggtctcaaccagaatgtttccaagcaccacaacgtcaaggtcca

SEQ ID NO: 552 - DNA, 1317 bp, artificial
  synthetic
tgtggttcttctgaaggtacaatttatttattaaactggaacggtttcggtgcaacatctgatcgtcaaacatctcgttctgaatgtcgtgatttcgttgttccaggttctaaaccatctggtaacaaacgtaaagattctcgtacagtttatcaatctcaatatacaaactggtctgttgaacaattagcagttcgtcgtttacgttataaaggtggtgatacattcacaggtttagcattaacacatgttatgtcttctacaacaacattaacaacaccacataaccaagttgatatgttattacaagaaatgaaacaagcatctttagaattaggtgaaggtaaattaccagcagcatgtttaaaaccagcattaggtgtttgtggtgaagcattctgtgaaaacttagaaggttctttcttatgtgtttgtgcagatgcaccagaagttattaaagcatctacattctgtaaaggttctgatgtttggtctttcggtgttgataacatgccattagaattatctggtcgtttacaaaaaacagttacaggtttattaattgaagaaattgaacgtcatggtgaaaaagttgaagattgtcaacgtttcgcaaaacaatatattaacaaacaaccaccacatcaagttgaagttgaagttgcagaacaaaacgtttctcgtatttgtcatttaggtaacggtacaattttatggattattgttttacaaccagcattacatcgtccaatgcatgttttagtttctttagattctgttgaaccaccagattggtctttcggtgaagaagaagatggtattttaacacaattacatcaagatatgcaacaattagaaattccacaagcatatcgtggtgcaccatctcgtccatctgatgcagcagatgaagttcgtgatacatctattaacgaatctcgtaactctcgtttcaaaggtaaacattctttagatggtgatgaagaagaagatgatgatgatggtggtacagcatatcgtttcttagaagaaatgcgttggcgtttaccattagcaaacatgtcttattatttctgtggtggtgcattaattcatccatgtcgtgttttaacagcagcacattgtacagatattttagttaaagaacaagcattcggtggtttatctcgtgcattagcattagattgtgaaatggttgaattaatggataaaccattaacattccgtattttatctgatttaattttcgatttcgaagatgcacgtagtataatcaactttgaaaaactgagtcatcatcatcatcatcattaataa

SEQ ID NO: 553 - protein, 437 aa, artificial
  synthetic
CGSSEGTIYLLNWNGFGATSDRQTSRSECRDFVVPGSKPSGNKRKDSRTVYQSQYTNWSVEQLAVRRLRYKGGDTFTGLALTHVMSSTTTLTTPHNQVDMLLQEMKQASLELGEGKLPAACLKPALGVCGEAFCENLEGSFLCVCADAPEVIKASTFCKGSDVWSFGVDNMPLELSGRLQKTVTGLLIEEIERHGEKVEDCQRFAKQYINKQPPHQVEVEVAEQNVSRICHLGNGTILWIIVLQPALHRPMHVLVSLDSVEPPDWSFGEEEDGILTQLHQDMQQLEIPQAYRGAPSRPSDAADEVRDTSINESRNSRFKGKHSLDGDEEEDDDDGGTAYRFLEEMRWRLPLANMSYYFCGGALIHPCRVLTAAHCTDILVKEQAFGGLSRALALDCEMVELMDKPLTFRILSDLIFDFEDARSIINFEKLSHHHHHH

SEQ ID NO: 554 - DNA, 1317 bp, artificial
  synthetic
ggttctggtgcattctctccagtttgtacatgtttaaaccgtggtcgtcgttctgaagatcaaccaccaccaccacgtttacaaggtccaccattccaaacaccattaccaggtccacatcatccaccattaccaacaacagttggtgatttctggcatatgatttgggaacaaaaatctacagttattgtttctcgtgcagcagttcattgtgcattatcttctccaccaccagaagcacgtgcattagcacgtcgtgatacattacgtttaatgtgttctcgtccatctccaacagcagaagttcgttggttccgtaaaggtaacgcatctaaagaacaagttggtttacatgatttagttcaacaattaccaaaaggtgttgatcaacaagatggtaaaaaccattctggttctccaggttgtgaagaatctgatgcattattagcagttacaggtggttgtttcatggaattatgtaaagttgcaaaatctgttgaaatggttgcaccaccacaaggtgttgttaaagcatcttattctttatctgaagatgcagaaggtgaatgtggttcttctgaaggtacaatttatttattaaactggaacggtttcggtgcaacatctgatttaaaaaacggtcgtttaacagttggtcaaaacattattttacatggtgcagaattagttggttctttacgtaacttatatacaccaaacgttatggtttctcgtttattaattttaggtggtgcattaggtgcacgtggttctggtgcagcagcaggtacagttcgtgaattattacaagatggttgtccaccattaccagatgtttctgcattctcttctggtcgtggtggtggtgaaggtcgtggttctgttgaagatgaacgtcaaatggatgatttacaaggtgcagcaccaccaattccaccacgtttaggtttaccagaagcaaaaattttctctggtacatcttctgaacaattcggttatgcagttcaaacatatcattctacatatttagttaaccaatctgcattcgatcaacgtgttgcaacagttcattctattttaggtgaagaaccacgtttcggtaaaacaccattagcaatgttagcagcaacatgtcatcgtggtgttggttctcaaacagcatctggtcgttctggtcaagttttaggtgttgttttaggttatcatgataacggtatgttctctccaattggtgaatcttgtgaagatattgatgaatgtgcacgtagtataatcaactttgaaaaactgagtcatcatcatcatcatcattaataa

SEQ ID NO: 555 - protein, 437 aa, artificial
  synthetic
GSGAFSPVCTCLNRGRRSEDQPPPPRLQGPPFQTPLPGPHHPPLPTTVGDFWHMIWEQKSTVIVSRAAVHCALSSPPPEARALARRDTLRLMCSRPSPTAEVRWFRKGNASKEQVGLHDLVQQLPKGVDQQDGKNHSGSPGCEESDALLAVTGGCFMELCKVAKSVEMVAPPQGVVKASYSLSEDAEGECGSSEGTIYLLNWNGFGATSDLKNGRLTVGQNIILHGAELVGSLRNLYTPNVMVSRLLILGGALGARGSGAAAGTVRELLQDGCPPLPDVSAFSSGRGGGEGRGSVEDERQMDDLQGAAPPIPPRLGLPEAKIFSGTSSEQFGYAVQTYHSTYLVNQSAFDQRVATVHSILGEEPRFGKTPLAMLAATCHRGVGSQTASGRSGQVLGVVLGYHDNGMFSPIGESCEDIDECARSIINFEKLSHHHHHH

SEQ ID NO: 556 - DNA, 1317 bp, artificial
  synthetic
catgcattaatgacatattctacagcacgtatgtctttcgtttgtccacgtttctatcgtaacggttctggtgcattctctccagtttgtacatgtttaaaccgtggtcgtcgttctgaagatcaattaaaaaacggtcgtttaacagttggtcaaaacattattttacatggtgcagaattagttggttatgaatctccaaacgaagaagaagaagcacatgttgaagatgatgcagattatgaaccaccagttccaaacttacattggttagttgttgaaaacgcaccacgtcgtacaccattaacagcacgtgcaaaaatgcgtcatggtttctctggtattttaattacagaaacaggtacaatgggttctaaaggtatttctacacgtttaccaggtccaggtaaaccagttccaggtccagcaggtccagcacaatctttacgtaacttatatacaccaaacgttatggtttctcgtttattaattttaggtggtgcatctcgtttcaaaggtaaacattctttagatggtgatgaagaagaagatgatgatgatggtggtacacaatcttctaaacgtttattaccatctgttggtttatctacagttttattaaaaaaagaatggaaacaagatcgtgaaacaggtcgtttcttacgtccatgtgaatgtttagttgttcgtgtttctcaatcttcttgttgtaaaccatgttgttgtcaatcttcttgttgtaaaccatgttgttgtaaattaggtttcgttaaattcgcacaaacaatgttcaaattaattacaggtacattatctaaagttgaatctaaagcagttaaattattatgttctgttttacgttctccacgttgtcgtttacaagttcaaggtcgtgttcaagaagaattagatcatgttttaggtccaggtcgtacaccacgtttaccaggtcaacgttgggtttggatgaacttccaatctccatctcattctccaggtttacgttctggtaacatggcagttattcattctcatttattaaaaggtttacaagaaaaattcttaaaaggtttattctatttattcgcatctttaacatctcatttaccatgggaacattgtggtaactggtggaacttacaattcgaatatattgatgatccatgggttcaacgtattgaaccagaatggtctatttctacagcagattatgcaatgttcaaagttgaaccagaagcagataaatatcgtttaacatatgcacgtagtataatcaactttgaaaaactgagtcatcatcatcatcatcattaataa

SEQ ID NO: 557 - protein, 437 aa, artificial
  synthetic
HALMTYSTARMSFVCPRFYRNGSGAFSPVCTCLNRGRRSEDQLKNGRLTVGQNIILHGAELVGYESPNEEEEAHVEDDADYEPPVPNLHWLVVENAPRRTPLTARAKMRHGFSGILITETGTMGSKGISTRLPGPGKPVPGPAGPAQSLRNLYTPNVMVSRLLILGGASRFKGKHSLDGDEEEDDDDGGTQSSKRLLPSVGLSTVLLKKEWKQDRETGRFLRPCECLVVRVSQSSCCKPCCCQSSCCKPCCCKLGFVKFAQTMFKLITGTLSKVESKAVKLLCSVLRSPRCRLQVQGRVQEELDHVLGPGRTPRLPGQRWVWMNFQSPSHSPGLRSGNMAVIHSHLLKGLQEKFLKGLFYLFASLTSHLPWEHCGNWWNLQFEYIDDPWVQRIEPEWSISTADYAMFKVEPEADKYRLTYARSIINFEKLSHHHHHH

SEQ ID NO: 558 - DNA, 1317 bp, artificial
  synthetic
gcaaaccgtgttgttatttgttatttcggtaaaggtgcagcatctgaaggtgatgcacatgcacatgcagaagcattattaaaagcagcaattcaatatggtacagaagaagatgttgttaaatctttaaaaaacggtcgtttaacagttggtcaaaacattattttacatggtgcagaattagttggtaaatctcgtgatgcagcacgttctcgtcgtcgtaaagaaaacttcgaattctatgaattagcatctacagcagattatgcaatgttcaaagttgaaccagaagcagataaatatcgtttaacatatgtttctcgtgcagcagttcattgtgcattatcttctccaccaccagaagcacgtgcattagcaaaaattttacaattatgtggtgaattatatcatttagatgcatcttctttacaattaaaagttcatccagcaccaccacatcatacaggtacagaatggcaaccagttgatccaggtcgtgcagcaattgaagttgaaaaaatgcgtcgtgcagaaacatctgcacgtgttgaaaactctccacaagttccagaaccaacatctggtgttattgcacgtgaagcagaaattatggaaacatctattcaagcaatgaaatatggtgcagatccatctttaattaacggtgaaggttgttcttgtattcatttagcagcaattttattattattacaattatgggattgtggtgcacaagaaacagataacgaacgttctgaatctttccgtgcagcaattgataaatcttgtgatggtccagaagaaattgaagcagatggtattacagttcatacaccattattaccatctgcaacaggtttattaaacgataacacattcgcattattctatttattcgcatctttaacatctcatttaccatgggaacattgtggtaactggtggggtttaccagaagcaaaaattttctctggtacatcttctgaacaattcggttatgcagttcaaacatatcattctacatatttagttaaccaatctgcattcgatcaacgtgttgcaacagttcatttatctgtttctcgtccagaacgtccaccacaatctgcagcattaggtggtttaggtccaggtgatggtttaccaggtccaccaggtacaatgatgatgttaccattccgttatggtggtgatggtcgtttcgcaccaccaaacatgcaagcagcacaagcagtttctcatattcaatcttctggtccagcacgtagtataatcaactttgaaaaactgagtcatcatcatcatcatcattaataa

SEQ ID NO: 559 - protein, 437 aa, artificial
  synthetic
ANRVVICYFGKGAASEGDAHAHAEALLKAAIQYGTEEDVVKSLKNGRLTVGQNIILHGAELVGKSRDAARSRRRKENFEFYELASTADYAMFKVEPEADKYRLTYVSRAAVHCALSSPPPEARALAKILQLCGELYHLDASSLQLKVHPAPPHHTGTEWQPVDPGRAAIEVEKMRRAETSARVENSPQVPEPTSGVIAREAEIMETSIQAMKYGADPSLINGEGCSCIHLAAILLLLQLWDCGAQETDNERSESFRAAIDKSCDGPEEIEADGITVHTPLLPSATGLLNDNTFALFYLFASLTSHLPWEHCGNWWGLPEAKIFSGTSSEQFGYAVQTYHSTYLVNQSAFDQRVATVHLSVSRPERPPQSAALGGLGPGDGLPGPPGTMMMLPFRYGGDGRFAPPNMQAAQAVSHIQSSGPARSIINFEKLSHHHHHH

SEQ ID NO: 560 - DNA, 1317 bp, artificial
  synthetic
ttattattacgtttaccatctttacgtacaatgtcttcttctgttattgaacaattattcttcgttcaaggtcgtgttcaagaagaattagatcatgttttaggtccaggtcgtacaccacgtttattaggtaacggtacaattttatggattattgttttacaaccagcattacatcgtccaatgcatgttaaagttgcagaaattacagaattaattttcaaagcattagaaaacgatcaaaaagttcgtattttaacacaattacatcaagatatgcaacaattagaaattccacaagcatatcgtggtgcatctaaacaacaagttgcagatgaagatgaaatgtggaaacaacgtcgtcgtcaacaatctgaagatttacatttcatgaaaaaaattccaccagttgcagaagcatctaacattttagttggtgaaattgttttaccatctaacgttattaacttctgtcaagcagaaaaaccagaaccaacaaaccaaggtttagaaaaaacagcatctatttctatttctgatccattcttccgttctgcacaacgttgggcaggttgtcaattatggttatgtacattattagcagttttaaaagttggttggttattcccaggtgttgatcaacaagatggtaaaaaccattctggttctccaggttgtgaagaatctgatgcagaaatgaacaaacaaatttctttattaacattcgcagttaaccatttaaaagcaaacgttaaattcaaatggtcttctgcattaacaacattaaaccaacataaacgtattcatacaggtgaaaaaacaacatctaaagattggcatcatggtggtccagaaaacattttattatgtacagattgtcgtcatccagcaccaccacatcatacaggtacagaatggcaaccagttgatccaggtcgtgcagcattatctgtttctcgtccagaacgtccaccacaatctgcagcattaggtggtttaggtccaggtcaagcatgtacatctggtggttgtttacatattttaccaattacagttacaacagcacaagcagttaaagcacaaacacgtgcagcattattattagttttacaagaagatgaagaacattggggtttatatttattatggttattattagttacacatgcatataactggaactgttgtttcattccaacaggtattgtttcttggggtcaaggttgtacaacagttggtcatttcggtgtttatacacgtgcacgtagtataatcaactttgaaaaactgagtcatcatcatcatcatcattaataa

SEQ ID NO: 561 - protein, 437 aa, artificial
  synthetic
LLLRLPSLRTMSSSVIEQLFFVQGRVQEELDHVLGPGRTPRLLGNGTILWIIVLQPALHRPMHVKVAEITELIFKALENDQKVRILTQLHQDMQQLEIPQAYRGASKQQVADEDEMWKQRRRQQSEDLHFMKKIPPVAEASNILVGEIVLPSNVINFCQAEKPEPTNQGLEKTASISISDPFFRSAQRWAGCQLWLCTLLAVLKVGWLFPGVDQQDGKNHSGSPGCEESDAEMNKQISLLTFAVNHLKANVKFKWSSALTTLNQHKRIHTGEKTTSKDWHHGGPENILLCTDCRHPAPPHHTGTEWQPVDPGRAALSVSRPERPPQSAALGGLGPGQACTSGGCLHILPITVTTAQAVKAQTRAALLLVLQEDEEHWGLYLLWLLLVTHAYNWNCCFIPTGIVSWGQGCTTVGHFGVYTRARSIINFEKLSHHHHHH

SEQ ID NO: 562 - DNA, 1317 bp, artificial
  synthetic
ttctcttctaacgttacaccaaaaatgttacaaattttcttatctgaaaaaaaatctatttctacatatcattctacatatttagttaaccaatctgcattcgatcaacgtgttgcaacagttcatttaacattaaacaacgatcaagttttcccaattaacccaccaaaattcgataaaattgaagatcaagcatgtacatctggtggttgtttacatattttaccaattacagttacaacagcacaagcatctacagcagattatgcaatgttcaaagttgaaccagaagcagataaatatcgtttaacatatcaacaacgtttacaagaagaattagatcataaattaggtccaggtgcatcttcttctcgtgttttctctgatttagataacatggcatctcgtggtcatgatcatttagataaccatcgtgaatctatttatattccagatgatattccaattccaacagaattcgaattacgtgaatctaacatgccaggtgaattaattcgtacacaaccacaacgtttcccacaattacaaacatctgcacaagttccaccaggtcaacgttgggtttggatgaacttccaatctccatctcattctccaggtttacgttctccatctcgtccatctgatgcagcagatgaagttcgtgatacatctattaacgaatctcgtaacgaaatgaacaaacaaatttctttattaacattcgcagttaaccatttaaaagcaaacgttaaaggtgttgatcaacaagatggtaaaaaccattctggttctccaggttgtgaagaatctgatgcattccgttatggtcatacattaattcaaccattaatgttccgtttagataaccgttatcaaccaattcgtaacgcagttgaaatgatgattgcatggttagatacacaattaaaaaacaaattaattggtttagaaaaaacagcatctatttctatttctgatccattcttccgttctgcacaacgttggcgtttaacatatccaggtattaaagttgttgttgttattgatggtaactctgaagatgatttaaaacaaccaccacatcaagttgaagttgaagttgcagaacaaaacgtttctcgtatttgtcatgcattaaacacatctacatgtccaccaaaccgtgttaaattagatattttcccaaaagaatgtgcaggtgaagcatctcgtttagcacattattctaaacgttctacaattacatctcgtgaaattgcacgtagtataatcaactttgaaaaactgagtcatcatcatcatcatcattaataa

SEQ ID NO: 563 - protein, 437 aa, artificial
  synthetic
FSSNVTPKMLQIFLSEKKSISTYHSTYLVNQSAFDQRVATVHLTLNNDQVFPINPPKFDKIEDQACTSGGCLHILPITVTTAQASTADYAMFKVEPEADKYRLTYQQRLQEELDHKLGPGASSSRVFSDLDNMASRGHDHLDNHRESIYIPDDIPIPTEFELRESNMPGELIRTQPQRFPQLQTSAQVPPGQRWVWMNFQSPSHSPGLRSPSRPSDAADEVRDTSINESRNEMNKQISLLTFAVNHLKANVKGVDQQDGKNHSGSPGCEESDAFRYGHTLIQPLMFRLDNRYQPIRNAVEMMIAWLDTQLKNKLIGLEKTASISISDPFFRSAQRWRLTYPGIKVVVVIDGNSEDDLKQPPHQVEVEVAEQNVSRICHALNTSTCPPNRVKLDIFPKECAGEASRLAHYSKRSTITSREIARSIINFEKLSHHHHHH

SEQ ID NO: 564 - DNA, 1317 bp, artificial
  synthetic
ttaaaaaacggtcgtttaacagttggtcaaaacattattttacatggtgcagaattagttggtatttatattccagatgatattccaattccaacagaattcgaattacgtgaatctaacatgccaaaacaagcatctttagaattaggtgaaggtaaattaccagcagcatgtttaaaaccagcattacgtttaacatatccaggtattaaagttgttgttgttattgatggtaactctgaagatgatttattccgttatggtcatacattaattcaaccattaatgttccgtttagataaccgttatcaaccatctttattattaaactcttctcaatcttcttcttctgaagaaacagttttacgttctgatgcatatttaaaacaatggcgttcttataacatttgtatttgggatacagcaggtcgtgaacaattcttctgtggtggtgcattaattcatccatgtcgtgttttaacagcagcacattgtacagatattcaagaatatttagcagcaaaaggtaaattaaactctcaaaacacaaaaccatatttaaaatctgatggtttaccaggtccaccaggtacaatgatgatgttaccattccgttatggtggtgatggtgaacaacgtcgtacagttcgtatttatttcgttggtccatctgcagttttaccagaagttgaatatgaatctccaaacgaagaagaagaagcacatgttgaagatgatgcagattatgaaccaccaaaccaattaaaagaaatttcttcttctacagattctcaaaaaaaagaagaaacaaaaggtaaaattacagttcatacaccattattaccatctgcaacaggtttattaaacgataacacattcgcaggtgaagttttaccaacaccaggtcaagaaccaccaacaccaattcatgatttatctgcatctgcagttcgtcgtttacgttataaaggtggtgatacattcacaggtttagcattaacacatgttcgtccattattaggtgatcgtcaatatattaaaaaattctatacagatgcagcattcttattagttcgtacatgtatgcataaccaaaaagatgttgttcaaatgccatctgaaacattaaaagcagttcgtcaagttccaggtttcggtgttgcacatgcattaggtaaccgtgttggtgaagcagcaatgccaacaattaacgaagatacaccaatgcgtcaacgtggttctcaatcttctggttctgatgcacgtagtataatcaactttgaaaaactgagtcatcatcatcatcatcattaataa

SEQ ID NO: 565 - protein, 437 aa, artificial
  synthetic
LKNGRLTVGQNIILHGAELVGIYIPDDIPIPTEFELRESNMPKQASLELGEGKLPAACLKPALRLTYPGIKVVVVIDGNSEDDLFRYGHTLIQPLMFRLDNRYQPSLLLNSSQSSSSEETVLRSDAYLKQWRSYNICIWDTAGREQFFCGGALIHPCRVLTAAHCTDIQEYLAAKGKLNSQNTKPYLKSDGLPGPPGTMMMLPFRYGGDGEQRRTVRIYFVGPSAVLPEVEYESPNEEEEAHVEDDADYEPPNQLKEISSSTDSQKKEETKGKITVHTPLLPSATGLLNDNTFAGEVLPTPGQEPPTPIHDLSASAVRRLRYKGGDTFTGLALTHVRPLLGDRQYIKKFYTDAAFLLVRTCMHNQKDVVQMPSETLKAVRQVPGFGVAHALGNRVGEAAMPTINEDTPMRQRGSQSSGSDARSIINFEKLSHHHHHH

SEQ ID NO: 566 - DNA, 1317 bp, artificial
  synthetic
attacagttcatacaccattattaccatctgcaacaggtttattaaacgataacacattcgcagaaccagatcgtaacgcattacgtcgtaaacaacgtgaacgtcgtaaccaagaaacacaacaagcaggttgtcaattatggttatgtacattattagcagttttaaaagttggttggttattcccagcaaaaatgcgtcatggtttctctggtattttaattacagaaacaggtacaatgggttctaaacgtttaacatatccaggtattaaagttgttgttgttattgatggtaactctgaagatgatttaccaaaattattacgtgaattagttccacaatatttaattcgtcaagtttctccagatgattggattcgtaacgcagttgaaatgatgattgcatggttagatacacaattaaaaaacaaattaattttagcagttgaattacaagcaggtgcaggtgaaccaggtttagcagcattagttgcattcgttaaattatcttctaaccgtgaacgtcatgttacaatgtgtgaagatttagaattacgtcgtgatgaattaatggataaaccattaacattccgtattttatctgatttaattttcgatttcgaagatccagaaccaaaaattattccatctgaagaaaacccaacattcgaagataacccagaagttccacgttctgcagaaccaggttatttagttacaatggttgttgcagttgatcgtgattctggtcaagatgaatatttaatttctcaatctgaacgttattctattacaatgacagaaaaatctacaattggtgaagttttaccaacaccaggtcaagaaccaccaacaccaattcatgatttatctgcatctaacttacaattcgaatatattgatgatccatgggttcaacgtattgaaccagaatggtctattcgttctattcaaggtccatctttatcttctcgtcgtaacgttatgtctgaagcatctttagatcaagaatatttagcagcaaaaggtaaattaaactctcaaaacacaaaaccatatttaaaatctcgttataacccatctttaaaaggtttaaacgttacaggttatgaaacacaagcaggtgaattcggtgaattaattcgtacacaaccacaacgtttcccacaattacaaacatctgcacaagttccagttcgtacatgtatgcataaccaaaaagatgttgttcaaatgccatctgaaacattaaaagcagcacgtagtataatcaactttgaaaaactgagtcatcatcatcatcatcattaataa

SEQ ID NO: 567 - protein, 437 aa, artificial
  synthetic
ITVHTPLLPSATGLLNDNTFAEPDRNALRRKQRERRNQETQQAGCQLWLCTLLAVLKVGWLFPAKMRHGFSGILITETGTMGSKRLTYPGIKVVVVIDGNSEDDLPKLLRELVPQYLIRQVSPDDWIRNAVEMMIAWLDTQLKNKLILAVELQAGAGEPGLAALVAFVKLSSNRERHVTMCEDLELRRDELMDKPLTFRILSDLIFDFEDPEPKIIPSEENPTFEDNPEVPRSAEPGYLVTMVVAVDRDSGQDEYLISQSERYSITMTEKSTIGEVLPTPGQEPPTPIHDLSASNLQFEYIDDPWVQRIEPEWSIRSIQGPSLSSRRNVMSEASLDQEYLAAKGKLNSQNTKPYLKSRYNPSLKGLNVTGYETQAGEFGELIRTQPQRFPQLQTSAQVPVRTCMHNQKDVVQMPSETLKAARSIINFEKLSHHHHHH

SEQ ID NO: 568 - DNA, 1317 bp, artificial
  synthetic
ggtgaattaattcgtacacaaccacaacgtttcccacaattacaaacatctgcacaagttccagcagttcgtcgtttacgttataaaggtggtgatacattcacaggtttagcattaacacatgttgaacaacgtcgtacagttcgtatttatttcgttggtccatctgcagttttaccagaagttgaatatccagaaattgttaaagaaaacaaattattagaacattattatcaagaattaaaaattgttaaaggtgcatgttggatttatgataacattaacatggcacatatgttagttgcaatttctgttggtttagaaaaaacagcatctatttctatttctgatccattcttccgttctgcacaacgttggattgttttaccatctaacgttattaacttctgtcaagcagaaaaaccagaaccaacaaaccaatatttaaaacaatggcgttcttataacatttgtatttgggatacagcaggtcgtgaacaattcgttttagtttctttagattctgttgaaccaccagattggtctttcggtgaagaagaagatggtatgttaattccattctgtgaagaattaaacttcaaaacaacatggttattccataaaacacgttctaacgaagttggtgaaggtccattctcttcttctgttgaattagcagttttaccaaaagaaaaatctacaacaaaacgttctccaaaaccacaaaacaaaaaaaaaacaaaaaaagttattgaagttaaagttgcagaaattacagaattaattttcaaagcattagaaaacgatcaaaaagttcgtccattaccagcacaagcacaatctgcatgggcaccagcaccagcaccagcaacatatcgtgcagaaattgaacgtcatggtgaaaaagttgaagattgtcaacgtttcgcaaaacaatatattaacacatatcattctacatatttagttaaccaatctgcattcgatcaacgtgttgcaacagttcatccatctgttccattaggtggtgcaccatcttgtggtatgttaatggataaaccacatccaccatatatgcatttcggtgaagttgcaaaacaaaaagaattcttcaacttatctcattgtcaattatctaaacaacaagttgcagatgaagatgaaatgtggaaacaacgtcgtcgtcaacaatctgaacaagcatgtacatctggtggttgtttacatattttaccaattacagttacaacagcacaagcagcacgtagtataatcaactttgaaaaactgagtcatcatcatcatcatcattaataa

SEQ ID NO: 569 - protein, 437 aa, artificial
  synthetic
GELIRTQPQRFPQLQTSAQVPAVRRLRYKGGDTFTGLALTHVEQRRTVRIYFVGPSAVLPEVEYPEIVKENKLLEHYYQELKIVKGACWIYDNINMAHMLVAISVGLEKTASISISDPFFRSAQRWIVLPSNVINFCQAEKPEPTNQYLKQWRSYNICIWDTAGREQFVLVSLDSVEPPDWSFGEEEDGMLIPFCEELNFKTTWLFHKTRSNEVGEGPFSSSVELAVLPKEKSTTKRSPKPQNKKKTKKVIEVKVAEITELIFKALENDQKVRPLPAQAQSAWAPAPAPATYRAEIERHGEKVEDCQRFAKQYINTYHSTYLVNQSAFDQRVATVHPSVPLGGAPSCGMLMDKPHPPYMHFGEVAKQKEFFNLSHCQLSKQQVADEDEMWKQRRRQQSEQACTSGGCLHILPITVTTAQAARSIINFEKLSHHHHHH

SEQ ID NO: 570 - DNA, 1317 bp, artificial
  synthetic
aaatctcgtgatgcagcacgttctcgtcgtcgtaaagaaaacttcgaattctatgaattagcaaacgaattatatttatctcaagaagatttctctattaacggtaacattaaagttttcccagattatatgcatttcggtgaagttgcaaaacaaaaagaattcttcaacttatctcattgtcaattaacaacatctaaagattggcatcatggtggtccagaaaacattttattatgtacagattgtcgtgttccaggtgatttatctcaaacatctgaagaacaatctttatctgatttcgaaatttctaacaactgttctttcgaagatttcacagaatctaaatctgttttagaagatggtcgtattcattgtgttaaagcagatccagcagcattaatggcattattacatttattaccatctccaacaccaaactatccagaaattgttaaagaaaacaaattattagaacattattatcaagaattaaaaattgttccaggtcaacgttgggtttggatgaacttccaatctccatctcattctccaggtttacgttctttctcttctaacgttacaccaaaaatgttacaaattttcttatctgaaaaaaaatctatttctttattagcagttacaggtggttgtttcatggaattatgtaaagttgcaaaatctgttgaaatggaaatgaacaaacaaatttctttattaacattcgcagttaaccatttaaaagcaaacgttaaaattaaagttaaatctgaagaaaaaattaaagcagttgaaaaatctgaaaaagaaacagttattaactatacaattttaatgcgtccacgtttcgttattttattaacagttatttcttggttcttaaaagatcatggtgaaccagcattaacagcatctgcaacagttttagtttctttagttgaatctgcaccagaagttattaaagcatctacattctgtaaaggttctgatgtttggtctttcggtgttggtccagcacattgtttattattaggtgaatgggttgcattaaacacattagcacgttgttctaaattaggtttcgttaaattcgcacaaacaatgttcaaattaattacaggtacattatctaaattattctatttattcgcatctttaacatctcatttaccatgggaacattgtggtaactggtggtataaaccagttcaacatgttacagcattatctacagtttcttcttgtcataaaatggtttctgcacgtagtataatcaactttgaaaaactgagtcatcatcatcatcatcattaataa

SEQ ID NO: 571 - protein, 437 aa, artificial
  synthetic
KSRDAARSRRRKENFEFYELANELYLSQEDFSINGNIKVFPDYMHFGEVAKQKEFFNLSHCQLTTSKDWHHGGPENILLCTDCRVPGDLSQTSEEQSLSDFEISNNCSFEDFTESKSVLEDGRIHCVKADPAALMALLHLLPSPTPNYPEIVKENKLLEHYYQELKIVPGQRWVWMNFQSPSHSPGLRSFSSNVTPKMLQIFLSEKKSISLLAVTGGCFMELCKVAKSVEMEMNKQISLLTFAVNHLKANVKIKVKSEEKIKAVEKSEKETVINYTILMRPRFVILLTVISWFLKDHGEPALTASATVLVSLVESAPEVIKASTFCKGSDVWSFGVGPAHCLLLGEWVALNTLARCSKLGFVKFAQTMFKLITGTLSKLFYLFASLTSHLPWEHCGNWWYKPVQHVTALSTVSSCHKMVSARSIINFEKLSHHHHHH

SEQ ID NO: 572 - DNA, 1317 bp, artificial
  synthetic
aacgaattatatttatctcaagaagatttctctattaacggtaacattaaagttttcccagatgttcgtggtcaatgtgatcgtcgtcgtcatgatacagcagcaacagaaattggtggtaaaatttctcaatcttcttgttgtaaaccatgttgttgtcaatcttcttgttgtaaaccatgttgttgtgatttatatatggatacaggtgaagaaatttgtttccgtgttgttgatgaatctttcgttgatatgtctgttggtaacattggtaactatttaacaacatctccagcatctttaaacccagaagaaaaacgtaaagattctcgtacagtttatcaatctcaatatacaaactggtctgttgaacaattaggtttcaacgaaaaccgtcaaaaacgtgcagttttagcagcacaattcgaagcaacatctccatgtggttcttctgaaggtacaatttatttattaaactggaacggtttcggtgcaacatctgattctttatctgttaaaattcgtgttatgccatgttctttcttcttattattacgtttcttcttaccaccattaccagatgtttctgcattctcttctggtcgtggtggtggtgaaggtcgtggttctcgtttcgcaccaccaaacatgcaagcagcacaagcagtttctcatattcaatcttctggtccagatttacatttcatgaaaaaaattccaccagttgcagaagcatctaacattttagttggtgaaaaacaagcatctttagaattaggtgaaggtaaattaccagcagcatgtttaaaaccagcattaacatatcattctacatatttagttaaccaatctgcattcgatcaacgtgttgcaacagttcattataaaccagttcaacatgttacagcattatctacagtttcttcttgtcataaaatggtttctggtttagaaaaaacagcatctatttctatttctgatccattcttccgttctgcacaacgttggtctttcccagcacaagatccatgtacaacaacatatgttgcagcaacagaaccagttccagaattattagcagttacaggtggttgtttcatggaattatgtaaagttgcaaaatctgttgaaatgatgcgtggtaaaccaaaagttattgatttaaaacgtaacgaagcaacagttgaaacattaacatctgaacgtaaaaaatggattcattgtttccaaggtgttacatgtattattttctgtgcagcagcacgtagtataatcaactttgaaaaactgagtcatcatcatcatcatcattaataa

SEQ ID NO: 573 - protein, 437 aa, artificial
  synthetic
NELYLSQEDFSINGNIKVFPDVRGQCDRRRHDTAATEIGGKISQSSCCKPCCCQSSCCKPCCCDLYMDTGEEICFRVVDESFVDMSVGNIGNYLTTSPASLNPEEKRKDSRTVYQSQYTNWSVEQLGFNENRQKRAVLAAQFEATSPCGSSEGTIYLLNWNGFGATSDSLSVKIRVMPCSFFLLLRFFLPPLPDVSAFSSGRGGGEGRGSRFAPPNMQAAQAVSHIQSSGPDLHFMKKIPPVAEASNILVGEKQASLELGEGKLPAACLKPALTYHSTYLVNQSAFDQRVATVHYKPVQHVTALSTVSSCHKMVSGLEKTASISISDPFFRSAQRWSFPAQDPCTTTYVAATEPVPELLAVTGGCFMELCKVAKSVEMMRGKPKVIDLKRNEATVETLTSERKKWIHCFQGVTCIIFCAAARSIINFEKLSHHHHHH

SEQ ID NO: 574 - DNA, 1317 bp, artificial
  synthetic
ttccgttatggtcatacattaattcaaccattaatgttccgtttagataaccgttatcaaccattaattttacaaggtaaccgtattcgttctgttacaaaaaaagcattcacaggtttagatgcaacattaccaccaaactctccaaacggttattgtggttctaacattaactatccagaatctcatggtccattaggtgcaggtattgcattagcatataaatataaaggtaacgatgaaatttgtttacaagaatatttagcagcaaaaggtaaattaaactctcaaaacacaaaaccatatttaaaatctacatctaactatacaggtttcgcaaacaaagatggttctaaaccatcttctttaggtgtttctccaccaggtttattagttttattatggcaagttacaacattaccagaacattgtttctcttggaacttacaattcgaatatattgatgatccatgggttcaacgtattgaaccagaatggtctatttctttcccagcacaagatccatgtacaacaacatatgttgcagcaacagaaccagttccagaagttcaaggtcgtgttcaagaagaattagatcatgttttaggtccaggtcgtacaccacgtttaccatctgaaggtgttcaagcagcaggtggtttagcacaaccattaggtgttccaccaggttctaaacaagcatctttagaattaggtgaaggtaaattaccagcagcatgtttaaaaccagcattagttaaagcacaaacacgtgcagcattattattagttttacaagaagatgaagaacattggggtcaatcttcttctcgtgcaaacttcgttattgcattaattaacattccagcagttgcattaggtaaaattttacaattatgtggtgaattatatcatttagatgcatcttctttacaattaaaagttgcaacaaaccaagcatctacaaaatctcataaatattctaaagttacaaactctttatctttatataaaccagttcaacatgttacagcattatctacagtttcttcttgtcataaaatggtttctgcaggttgtcaattatggttatgtacattattagcagttttaaaagttggttggttattcccaggtttcaacgaaaaccgtcaaaaacgtgcagttttagcagcacaattcgaagcaacatctccagttcgtacatgtatgcataaccaaaaagatgttgttcaaatgccatctgaaacattaaaagcagcacgtagtataatcaactttgaaaaactgagtcatcatcatcatcatcattaataa

SEQ ID NO: 575 - protein, 437 aa, artificial
  synthetic
FRYGHTLIQPLMFRLDNRYQPLILQGNRIRSVTKKAFTGLDATLPPNSPNGYCGSNINYPESHGPLGAGIALAYKYKGNDEICLQEYLAAKGKLNSQNTKPYLKSTSNYTGFANKDGSKPSSLGVSPPGLLVLLWQVTTLPEHCFSWNLQFEYIDDPWVQRIEPEWSISFPAQDPCTTTYVAATEPVPEVQGRVQEELDHVLGPGRTPRLPSEGVQAAGGLAQPLGVPPGSKQASLELGEGKLPAACLKPALVKAQTRAALLLVLQEDEEHWGQSSSRANFVIALINIPAVALGKILQLCGELYHLDASSLQLKVATNQASTKSHKYSKVTNSLSLYKPVQHVTALSTVSSCHKMVSAGCQLWLCTLLAVLKVGWLFPGFNENRQKRAVLAAQFEATSPVRTCMHNQKDVVQMPSETLKAARSIINFEKLSHHHHHH

SEQ ID NO: 576 - DNA, 1317 bp, artificial
  synthetic
gattctgaaggtccagcagaaggtgaaggtcatttaggtcgttgtatgcgtcgtttacgtgatacattagttgattctcgtacatctgttacaaacgttaaattcgcaccaaaacatatgggtttatctattttaggtgaagaaccacgtttcggtaaaacaccattagcaatgttagcagcaacatgtccaccaggtttattagttttattatggcaagttacaacattaccagaacattgtttctcttggaaatctacaacaaaacgttctccaaaaccacaaaacaaaaaaaaaacaaaaaaagttattgaacgtcgtgatacattacgtttaatgtgttctcgtccatctccaacagcagaagttcgttggttcttagttttagcagtttctatgacaaaaacaaaaaacgaagtttgtggttatccagttattgtttatatgcatttcggtgaagttgcaaaacaaaaagaattcttcaacttatctcattgtcaattaccaggtcaacgttgggtttggatgaacttccaatctccatctcattctccaggtttacgttctcaatcttcttgtcaaccagcatgttgtacaacatcttcttgtcaacaagcatgttgtgttccagcaaaaatgcgtcatggtttctctggtattttaattacagaaacaggtacaatgggttctaaaaaacgtaaagattctcgtacagtttatcaatctcaatatacaaactggtctgttgaacaattaggtttcaacgaaaaccgtcaaaaacgtgcagttttagcagcacaattcgaagcaacatctccaggtttagaaaaaacagcatctatttctatttctgatccattcttccgttctgcacaacgttggccacgtccagatattttagatatgccatctcaacatgttgcattcccatcttctccacaaattggtgtttgtggtgaagcattctgtgaaaacttagaaggttctttcttatgtgtttgtgcagataaacaagcatctttagaattaggtgaaggtaaattaccagcagcatgtttaaaaccagcattattaaacattccagcaccattacatccagaaaaaacagttaacatgacatatcaaccaacaacacaacaacgtttacaagaagaattagatcataaattaggtccaggtgcatcttcttctcgtgttcaagaatatttagcagcaaaaggtaaattaaactctcaaaacacaaaaccatatttaaaatctgcacgtagtataatcaactttgaaaaactgagtcatcatcatcatcatcattaataa

SEQ ID NO: 577 - protein, 437 aa, artificial
  synthetic
DSEGPAEGEGHLGRCMRRLRDTLVDSRTSVTNVKFAPKHMGLSILGEEPRFGKTPLAMLAATCPPGLLVLLWQVTTLPEHCFSWKSTTKRSPKPQNKKKTKKVIERRDTLRLMCSRPSPTAEVRWFLVLAVSMTKTKNEVCGYPVIVYMHFGEVAKQKEFFNLSHCQLPGQRWVWMNFQSPSHSPGLRSQSSCQPACCTTSSCQQACCVPAKMRHGFSGILITETGTMGSKKRKDSRTVYQSQYTNWSVEQLGFNENRQKRAVLAAQFEATSPGLEKTASISISDPFFRSAQRWPRPDILDMPSQHVAFPSSPQIGVCGEAFCENLEGSFLCVCADKQASLELGEGKLPAACLKPALLNIPAPLHPEKTVNMTYQPTTQQRLQEELDHKLGPGASSSRVQEYLAAKGKLNSQNTKPYLKSARSIINFEKLSHHHHHH

SEQ ID NO: 578 - DNA, 1317 bp, artificial
  synthetic
ccagttgaaattgcaattttccaaccacaaaacggtcaattcattcatttctatcgtgaaccatctttatctgttaaaattcgtgttatgccatgttctttcttcttattattacgtttcttcttacaacaacgtttacaagaagaattagatcataaattaggtccaggtgcatcttcttctcgtgttgtttctaacttagaagaattaccattaaaccaatatcgtgaatatattaaagttcaaacagataaatctttatctgatttacgtggttatgtttatcaaggtttaaaatctggtaacgtttctattatttatattccagatgatattccaattccaacagaattcgaattacgtgaatctaacatgccaccagaaccaaaaattattccatctgaagaaaacccaacattcgaagataacccagaagttccaatgttaattccattctgtgaagaattaaacttcaaaacaacatggttattccataaaacacgttgggttttaacagcagcacattgtattcgtaacaaatctgttattttattaggtcgtcattctgttcatgcacgtgcatgtggtcgtttcttccataaaggtcaaggtacatgtttctatttcggtttagcagttgaattacaagcaggtgcaggtgaaccaggtttagcagcattagttgcattcgttaacgaattatatttatctcaagaagatttctctattaacggtaacattaaagttttcccagataactctattcatgcaaaaattacatcttataaattcgaatctgttaacttcgattctaaaattttcaaatggtcttctgcattaacaacattaaaccaacataaacgtattcatacaggtgaaaaagcattccatgcattcgcacaaccattcgcattaccacattctgcaatgttattcgttcaagcagcagcaccattcaaagcaccagaattattaaaaggtcaatctgaagatgaacaaccagatgcattaccaccaaacttctcttctttagaaaacttccgtgcatatgttgatggtttcgaaccaaacttctctgatttagataacatggcatctcgtggtcatgatcatttagataaccatcgtgaatctacatatcattctacatatttagttaaccaatctgcattcgatcaacgtgttgcaacagttcatattcgtaacgcagttgaaatgatgattgcatggttagatacacaattaaaaaacaaattaattgcacgtagtataatcaactttgaaaaactgagtcatcatcatcatcatcattaataa

SEQ ID NO: 579 - protein, 437 aa, artificial
  synthetic
PVEIAIFQPQNGQFIHFYREPSLSVKIRVMPCSFFLLLRFFLQQRLQEELDHKLGPGASSSRVVSNLEELPLNQYREYIKVQTDKSLSDLRGYVYQGLKSGNVSIIYIPDDIPIPTEFELRESNMPPEPKIIPSEENPTFEDNPEVPMLIPFCEELNFKTTWLFHKTRWVLTAAHCIRNKSVILLGRHSVHARACGRFFHKGQGTCFYFGLAVELQAGAGEPGLAALVAFVNELYLSQEDFSINGNIKVFPDNSIHAKITSYKFESVNFDSKIFKWSSALTTLNQHKRIHTGEKAFHAFAQPFALPHSAMLFVQAAAPFKAPELLKGQSEDEQPDALPPNFSSLENFRAYVDGFEPNFSDLDNMASRGHDHLDNHRESTYHSTYLVNQSAFDQRVATVHIRNAVEMMIAWLDTQLKNKLIARSIINFEKLSHHHHHH

SEQ ID NO: 580 - DNA, 1317 bp, artificial
  synthetic
acaacatctaaagattggcatcatggtggtccagaaaacattttattatgtacagattgtcgtcgttataacccatctttaaaaggtttaaacgttacaggttatgaaacacaagcaggtgaattcgtttctcgtgcagcagttcattgtgcattatcttctccaccaccagaagcacgtgcattagcagttcaattacgtgatggtgattctaactgtcaatggtctgttttatatgttattattgcaacagaaatgaacaaacaaatttctttattaacattcgcagttaaccatttaaaagcaaacgttaaagcatggcaaaactctattcgtcataacttatgtttaaacgattgtttcgttaaaattccacgtgatggtttaccaggtccaccaggtacaatgatgatgttaccattccgttatggtggtgatggtattacagttcatacaccattattaccatctgcaacaggtttattaaacgataacacattcgcagcaggtgaagcatctcgtttagcacattattctaaacgttctacaattacatctcgtgaaattggtgaagttttaccaacaccaggtcaagaaccaccaacaccaattcatgatttatctgcatctgaacgtacaacagaagaagttgcacgtaaccaacgtgcagttgaagcagaaaaatgtgcacgttctttcccagcacaagatccatgtacaacaacatatgttgcagcaacagaaccagttccagaaccatctgttccattaggtggtgcaccatcttgtggtatgttaatggataaaccacatccaccatctttatctgttaaaattcgtgttatgccatgttctttcttcttattattacgtttcttcttatctaaacaacaagttgcagatgaagatgaaatgtggaaacaacgtcgtcgtcaacaatctgaaccaccattaccagatgtttctgcattctcttctggtcgtggtggtggtgaaggtcgtggttctaaccaaccacgtttaattcaatgtgaaaacaaagttggtaaattattattcattacagaaattaaatctacaacaaaacgttctccaaaaccacaaaacaaaaaaaaaacaaaaaaagttattgaaaaacgtaaagattctcgtacagtttatcaatctcaatatacaaactggtctgttgaacaattaaacttacaattcgaatatattgatgatccatgggttcaacgtattgaaccagaatggtctattgcacgtagtataatcaactttgaaaaactgagtcatcatcatcatcatcattaataa

SEQ ID NO: 581 - protein, 437 aa, artificial
  synthetic
TTSKDWHHGGPENILLCTDCRRYNPSLKGLNVTGYETQAGEFVSRAAVHCALSSPPPEARALAVQLRDGDSNCQWSVLYVIIATEMNKQISLLTFAVNHLKANVKAWQNSIRHNLCLNDCFVKIPRDGLPGPPGTMMMLPFRYGGDGITVHTPLLPSATGLLNDNTFAAGEASRLAHYSKRSTITSREIGEVLPTPGQEPPTPIHDLSASERTTEEVARNQRAVEAEKCARSFPAQDPCTTTYVAATEPVPEPSVPLGGAPSCGMLMDKPHPPSLSVKIRVMPCSFFLLLRFFLSKQQVADEDEMWKQRRRQQSEPPLPDVSAFSSGRGGGEGRGSNQPRLIQCENKVGKLLFITEIKSTTKRSPKPQNKKKTKKVIEKRKDSRTVYQSQYTNWSVEQLNLQFEYIDDPWVQRIEPEWSIARSIINFEKLSHHHHHH

SEQ ID NO: 582 - DNA, 1317 bp, artificial
  synthetic
gaattaatggataaaccattaacattccgtattttatctgatttaattttcgatttcgaagatcaagcatgtacatctggtggttgtttacatattttaccaattacagttacaacagcacaagcagttgaagatgaacgtcaaatggatgatttacaaggtgcagcaccaccaattccaccacgtttaaactatacaattttaatgcgtccacgtttcgttattttattaacagttatttcttggttcttaacatctaactatacaggtttcgcaaacaaagatggttctaaaccatcttctttaggtgtttctaaattaggtatttctttctctgaagatcaaaaaaaagaaaaagatcaattaggtaaagcaccagcagcaccattcaaagcaccagaattattaaaaggtcaatctgaagatgaacaaccagatgcagcaacaaaccaagcatctacaaaatctcataaatattctaaagttacaaactctttatctttagtttctaacttagaagaattaccattaaaccaatatcgtgaatatattaaagttcaaacagatgcaggttgtcaattatggttatgtacattattagcagttttaaaagttggttggttattcccaccatctcgtccatctgatgcagcagatgaagttcgtgatacatctattaacgaatctcgtaacacatatcattctacatatttagttaaccaatctgcattcgatcaacgtgttgcaacagttcatttctgtggtggtgcattaattcatccatgtcgtgttttaacagcagcacattgtacagatattatggcattcgtttatttacgtgttttccgtaaagcacaaaaacaagttaaaaaaattgattctacaacaacattacgtgaatggtctacaatttgtcgtcaattatatgttcaagataaccgtgaatctcaatcttcttgttgtaaaccatgttgttgtcaatcttcttgttgtaaaccatgttgttgtttaggtaacggtacaattttatggattattgttttacaaccagcattacatcgtccaatgcatttagttttagcagtttctatgacaaaaacaaaaaacgaagtttgtggttatccagttattgttttctcttctaacgttacaccaaaaatgttacaaattttcttatctgaaaaaaaatctatttctattgttttaccatctaacgttattaacttctgtcaagcagaaaaaccagaaccaacaaaccaagcacgtagtataatcaactttgaaaaactgagtcatcatcatcatcatcattaataa

SEQ ID NO: 583 - protein, 437 aa, artificial
  synthetic
ELMDKPLTFRILSDLIFDFEDQACTSGGCLHILPITVTTAQAVEDERQMDDLQGAAPPIPPRLNYTILMRPRFVILLTVISWFLTSNYTGFANKDGSKPSSLGVSKLGISFSEDQKKEKDQLGKAPAAPFKAPELLKGQSEDEQPDAATNQASTKSHKYSKVTNSLSLVSNLEELPLNQYREYIKVQTDAGCQLWLCTLLAVLKVGWLFPPSRPSDAADEVRDTSINESRNTYHSTYLVNQSAFDQRVATVHFCGGALIHPCRVLTAAHCTDIMAFVYLRVFRKAQKQVKKIDSTTTLREWSTICRQLYVQDNRESQSSCCKPCCCQSSCCKPCCCLGNGTILWIIVLQPALHRPMHLVLAVSMTKTKNEVCGYPVIVFSSNVTPKMLQIFLSEKKSISIVLPSNVINFCQAEKPEPTNQARSIINFEKLSHHHHHH

SEQ ID NO: 584 - DNA, 1317 bp, artificial
  synthetic
ggtaacatggcagttattcattctcatttattaaaaggtttacaagaaaaattcttaaaaggtgcaccagaagttattaaagcatctacattctgtaaaggttctgatgtttggtctttcggtgttgcattccatgcattcgcacaaccattcgcattaccacattctgcaatgttattcgttcaagcattatctgtttctcgtccagaacgtccaccacaatctgcagcattaggtggtttaggtccaggtgttgcattagaattaaacggtggtttatgtgttgaagcatgtggtgcattagaaggtttatggaaaattttacaattatgtggtgaattatatcatttagatgcatcttctttacaattaaaagttccaaaattattacgtgaattagttccacaatatttaattcgtcaagtttctccagatgattggcatgcattaatgacatattctacagcacgtatgtctttcgtttgtccacgtttctatcgtaacccaggtcaacgttgggtttggatgaacttccaatctccatctcattctccaggtttacgttctgttccaaacttacattggttagttgttgaaaacgcaccacgtcgtacaccattaacagcacgtaaattatcttctaaccgtgaacgtcatgttacaatgtgtgaagatttagaattacgtcgtgatttattagcagttacaggtggttgtttcatggaattatgtaaagttgcaaaatctgttgaaatgcaatcttcttctcgtgcaaacttcgttattgcattaattaacattccagcagttgcattaggtggttatcatgataacggtatgttctctccaattggtgaatcttgtgaagatattgatgaatgtggtatttctacacgtttaccaggtccaggtaaaccagttccaggtccagcaggtccagcacaaattttaacacaattacatcaagatatgcaacaattagaaattccacaagcatatcgtggtgcattaattttacaaggtaaccgtattcgttctgttacaaaaaaagcattcacaggtttagatgcaatgaaatatggtgcagatccatctttaattaacggtgaaggttgttcttgtattcatttagcaggtttcaacgaaaaccgtcaaaaacgtgcagttttagcagcacaattcgaagcaacatctccaatgccaacaattaacgaagatacaccaatgcgtcaacgtggttctcaatcttctggttctgatgcacgtagtataatcaactttgaaaaactgagtcatcatcatcatcatcattaataa

SEQ ID NO: 585 - protein, 437 aa, artificial
  synthetic
GNMAVIHSHLLKGLQEKFLKGAPEVIKASTFCKGSDVWSFGVAFHAFAQPFALPHSAMLFVQALSVSRPERPPQSAALGGLGPGVALELNGGLCVEACGALEGLWKILQLCGELYHLDASSLQLKVPKLLRELVPQYLIRQVSPDDWHALMTYSTARMSFVCPRFYRNPGQRWVWMNFQSPSHSPGLRSVPNLHWLVVENAPRRTPLTARKLSSNRERHVTMCEDLELRRDLLAVTGGCFMELCKVAKSVEMQSSSRANFVIALINIPAVALGGYHDNGMFSPIGESCEDIDECGISTRLPGPGKPVPGPAGPAQILTQLHQDMQQLEIPQAYRGALILQGNRIRSVTKKAFTGLDAMKYGADPSLINGEGCSCIHLAGFNENRQKRAVLAAQFEATSPMPTINEDTPMRQRGSQSSGSDARSIINFEKLSHHHHHH

SEQ ID NO: 586 - DNA, 1317 bp, artificial
  synthetic
gttccaggtgatttatctcaaacatctgaagaacaatctttatctgatttcgaaatttctaacttctctgatttagataacatggcatctcgtggtcatgatcatttagataaccatcgtgaatctggtccagcacattgtttattattaggtgaatgggttgcattaaacacattagcacgttgttcttctcgtttcaaaggtaaacattctttagatggtgatgaagaagaagatgatgatgatggtggtgcagaaaacacacgtttatgtacaaaagcaccaggtccatctcgtacagaatctacacaacgtgttttctgtgaaatgcgtccagttttagaatgtgaagaagaattatattcttcttgtcgtcaaccattaccagcacaagcacaatctgcatgggcaccagcaccagcaccagcaacatatcgtgcaccattcccagcagatggtttaggtgcagaagatgcattccaatgtttagaagaaacaccattcttaacattaaacaacgatcaagttttcccaattaacccaccaaaattcgataaaattgaagatgttcatgcacgtgcatgtggtcgtttcttccataaaggtcaaggtacatgtttctatttcggtcatccagcaccaccacatcatacaggtacagaatggcaaccagttgatccaggtcgtgcagcagaacaacgtcgtacagttcgtatttatttcgttggtccatctgcagttttaccagaagttgaaccaccaccaccaccattaccagcagcaccacaagcaccagaattcccatggatgaaagaaaaattccgttatggtcatacattaattcaaccattaatgttccgtttagataaccgttatcaaccaattcgtaacgcagttgaaatgatgattgcatggttagatacacaattaaaaaacaaattaattatgtcttctacaacaacattaacaacaccacataaccaagttgatatgttattacaagaaatgacattagttgattctcgtacatctgttacaaacgttaaattcgcaccaaaacatatgggtttatggaaacaagatcgtgaaacaggtcgtttcttacgtccatgtgaatgtttagttgttcgtgttttattaaaaggtccaccaccattcgaagattctttagcattcgatggtttatctttagcacgttatatgcatttcggtgaagttgcaaaacaaaaagaattcttcaacttatctcattgtcaattagcacgtagtataatcaactttgaaaaactgagtcatcatcatcatcatcattaataa

SEQ ID NO: 587 - protein, 437 aa, artificial
  synthetic
VPGDLSQTSEEQSLSDFEISNFSDLDNMASRGHDHLDNHRESGPAHCLLLGEWVALNTLARCSSRFKGKHSLDGDEEEDDDDGGAENTRLCTKAPGPSRTESTQRVFCEMRPVLECEEELYSSCRQPLPAQAQSAWAPAPAPATYRAPFPADGLGAEDAFQCLEETPFLTLNNDQVFPINPPKFDKIEDVHARACGRFFHKGQGTCFYFGHPAPPHHTGTEWQPVDPGRAAEQRRTVRIYFVGPSAVLPEVEPPPPPLPAAPQAPEFPWMKEKFRYGHTLIQPLMFRLDNRYQPIRNAVEMMIAWLDTQLKNKLIMSSTTTLTTPHNQVDMLLQEMTLVDSRTSVTNVKFAPKHMGLWKQDRETGRFLRPCECLVVRVLLKGPPPFEDSLAFDGLSLARYMHFGEVAKQKEFFNLSHCQLARSIINFEKLSHHHHHH

SEQ ID NO: 588 - DNA, 1317 bp, artificial
  synthetic
ttagcagttgaattacaagcaggtgcaggtgaaccaggtttagcagcattagttgcattcgttttcaaatggtcttctgcattaacaacattaaaccaacataaacgtattcatacaggtgaaaaacgtttcgcaccaccaaacatgcaagcagcacaagcagtttctcatattcaatcttctggtccagttcgtcaagttccaggtttcggtgttgcacatgcattaggtaaccgtgttggtgaagcagcagcaaaccgtgttgttatttgttatttcggtaaaggtgcagcatctgaaggtgatgcacatgcacgtcgtgatacattacgtttaatgtgttctcgtccatctccaacagcagaagttcgttggttcacagttccaaaagaaccagcatctgcaccagaagcaccaacattaccaacaacagcaccagcagttaaagcacaaacacgtgcagcattattattagttttacaagaagatgaagaacattggggtttatctcaaaacccaccatctcaaattttccattctttatctggtaactctcgtccattacaagttgaatctaaagcagttaaattattatgttctgttttacgttctccacgttgtcgtttacaaccatatgaatgtaacatttgtgaaaaagcattatctcatcgtggttctttaacattacatcaaatgtctgttggtaacattggtaactatttaacaacatctccagcatctttaaacccagaagaagttttagtttctttagattctgttgaaccaccagattggtctttcggtgaagaagaagatggtccagaaccaacatctggtgttattgcacgtgaagcagaaattatggaaacatctattcaagcaggtgtttgtggtgaagcattctgtgaaaacttagaaggttctttcttatgtgtttgtgcagatgaaccagatcgtaacgcattacgtcgtaaacaacgtgaacgtcgtaaccaagaaacacaacaacaagaatatttagcagcaaaaggtaaattaaactctcaaaacacaaaaccatatttaaaatctaaacaaccaccacatcaagttgaagttgaagttgcagaacaaaacgtttctcgtatttgtcattctttacgtaacttatatacaccaaacgttatggtttctcgtttattaattttaggtggtgcagatttacatttcatgaaaaaaattccaccagttgcagaagcatctaacattttagttggtgaagcacgtagtataatcaactttgaaaaactgagtcatcatcatcatcatcattaataa

SEQ ID NO: 589 - protein, 437 aa, artificial
  synthetic
LAVELQAGAGEPGLAALVAFVFKWSSALTTLNQHKRIHTGEKRFAPPNMQAAQAVSHIQSSGPVRQVPGFGVAHALGNRVGEAAANRVVICYFGKGAASEGDAHARRDTLRLMCSRPSPTAEVRWFTVPKEPASAPEAPTLPTTAPAVKAQTRAALLLVLQEDEEHWGLSQNPPSQIFHSLSGNSRPLQVESKAVKLLCSVLRSPRCRLQPYECNICEKALSHRGSLTLHQMSVGNIGNYLTTSPASLNPEEVLVSLDSVEPPDWSFGEEEDGPEPTSGVIAREAEIMETSIQAGVCGEAFCENLEGSFLCVCADEPDRNALRRKQRERRNQETQQQEYLAAKGKLNSQNTKPYLKSKQPPHQVEVEVAEQNVSRICHSLRNLYTPNVMVSRLLILGGADLHFMKKIPPVAEASNILVGEARSIINFEKLSHHHHHH

SEQ ID NO: 590 - DNA, 1317 bp, artificial
  synthetic
aacggtgttgtttctttagttggtggtccatgtacattaacatctgcaggtcaattattaccatctattttaggtgaagaaccacgtttcggtaaaacaccattagcaatgttagcagcaacatgtgatgaatatttaatttctcaatctgaacgttattctattacaatgacagaaaaatctacaattttatgtgatttatctgatttagaatatttacatgaagaattccatcaatctttacaatggatggaatctttccgtgcagcaattgataaatcttgtgatggtccagaagaaattgaagcagatggtacattaattcaaggtgttttacgtattaactctaaaaaattccatgaagcattcattccatctaaacaagcatctttagaattaggtgaaggtaaattaccagcagcatgtttaaaaccagcattagattctgaaggtccagcagaaggtgaaggtcatttaggtcgttgtatgcgtcgtttacgtgatgcagttcgtcgtttacgttataaaggtggtgatacattcacaggtttagcattaacacatgttgttcgtacatgtatgcataaccaaaaagatgttgttcaaatgccatctgaaacattaaaagcagataacatgccattagaattatctggtcgtttacaaaaaacagttacaggtttattaattgaacaatcttcttgtcaaccagcatgttgtacaacatcttcttgtcaacaagcatgttgtgttccaattaaagttaaatctgaagaaaaaattaaagcagttgaaaaatctgaaaaagaaacagttattgttcgtcgtacaaaattacaaatgcaaacagcagcattagaattccgtcaacgtattattttagatttatatatggatacaggtgaagaaatttgtttccgtgttgttgatgaatctttcgttgatgcattaaacacatctacatgtccaccaaaccgtgttaaattagatattttcccaaaagaatgtgaatatgttaaatggttcttacatgcagatttatcttcttatttaacaccaatgaaacatatgatttctcgtgatgcagcacaaaaaatttatttagaaatgaaattcacattacgtgattgtaacaactgttctttcgaagatttcacagaatctaaatctgttttagaagatggtcgtattcattgttctaaaaaattcttagaaaacttattagaaattttcaactctcatgttgatcatggtacaggtgcacgtagtataatcaactttgaaaaactgagtcatcatcatcatcatcattaataa

SEQ ID NO: 591 - protein, 437 aa, artificial
  synthetic
NGVVSLVGGPCTLTSAGQLLPSILGEEPRFGKTPLAMLAATCDEYLISQSERYSITMTEKSTILCDLSDLEYLHEEFHQSLQWMESFRAAIDKSCDGPEEIEADGTLIQGVLRINSKKFHEAFIPSKQASLELGEGKLPAACLKPALDSEGPAEGEGHLGRCMRRLRDAVRRLRYKGGDTFTGLALTHVVRTCMHNQKDVVQMPSETLKADNMPLELSGRLQKTVTGLLIEQSSCQPACCTTSSCQQACCVPIKVKSEEKIKAVEKSEKETVIVRRTKLQMQTAALEFRQRIILDLYMDTGEEICFRVVDESFVDALNTSTCPPNRVKLDIFPKECEYVKWFLHADLSSYLTPMKHMISRDAAQKIYLEMKFTLRDCNNCSFEDFTESKSVLEDGRIHCSKKFLENLLEIFNSHVDHGTGARSIINFEKLSHHHHHH

SEQ ID NO: 592 - DNA, 1317 bp, artificial
  synthetic
tcttcttatgttaaattatctgttggtaaaaacacacatacatctaaaacatgtccacataaccatcgtggtgttggttctcaaacagcatctggtcgttctggtcaagttttaggtgttgttttattaacacaagatattattatgaaaattttacaaaaaaaatctgttgaagttaacttcacagaaatttatattccagatgatattccaattccaacagaattcgaattacgtgaatctaacatgccacgtccattattaggtgatcgtcaatatattaaaaaattctatacagatgcagcattcttattaccatctgaaggtgttcaagcagcaggtggtttagcacaaccattaggtgttccaccaggttcttctttagattctttcggttctcgttctcgtcatacaccatctccagatgttgttttacgtggtccattaccaacaacagttggtgatttctggcatatgatttgggaacaaaaatctacagttattggtccattaggtgcaggtattgcattagcatataaatataaaggtaacgatgaaatttgtttagttaaagttgcagaaattacagaattaattttcaaagcattagaaaacgatcaaaaagttcgtttagaagaattatatgcattattcaaagcacaacatttaacatcttgttattggggtggttcttctacagcagattatgcaatgttcaaagttgaaccagaagcagataaatatcgtttaacatatgttgattctttcttacaagcagatggtccacaagaacgtccaccagcagcattaccagaatcttatccagaaattgttaaagaaaacaaattattagaacattattatcaagaattaaaaattgttaacgaattatatttatctcaagaagatttctctattaacggtaacattaaagttttcccagatggtttaccagaagcaaaaattttctctggtacatcttctgaacaattcggttatgcagttcaatataaaccagttcaacatgttacagcattatctacagtttcttcttgtcataaaatggtttctttattattacgtttaccatctttacgtacaatgtcttcttctgttattgaacaattattcttcaaaggtgcatgttggatttatgataacattaacatggcacatatgttagttgcaatttctgtttgtggttcttctgaaggtacaatttatttattaaactggaacggtttcggtgcaacatctgatgcacgtagtataatcaactttgaaaaactgagtcatcatcatcatcatcattaataa

SEQ ID NO: 593 - protein, 437 aa, artificial
  synthetic
SSYVKLSVGKNTHTSKTCPHNHRGVGSQTASGRSGQVLGVVLLTQDIIMKILQKKSVEVNFTEIYIPDDIPIPTEFELRESNMPRPLLGDRQYIKKFYTDAAFLLPSEGVQAAGGLAQPLGVPPGSSLDSFGSRSRHTPSPDVVLRGPLPTTVGDFWHMIWEQKSTVIGPLGAGIALAYKYKGNDEICLVKVAEITELIFKALENDQKVRLEELYALFKAQHLTSCYWGGSSTADYAMFKVEPEADKYRLTYVDSFLQADGPQERPPAALPESYPEIVKENKLLEHYYQELKIVNELYLSQEDFSINGNIKVFPDGLPEAKIFSGTSSEQFGYAVQYKPVQHVTALSTVSSCHKMVSLLLRLPSLRTMSSSVIEQLFFKGACWIYDNINMAHMLVAISVCGSSEGTIYLLNWNGFGATSDARSIINFEKLSHHHHHH

SEQ ID NO: 594 - DNA, 1317 bp, artificial
  synthetic
aacggtgttgtttctttagttggtggtccatgtacattaacatctgcaggtcaattattaccatctattttaggtgaagaaccacgtttcggtaaaacaccattagcaatgttagcagcaacatgtgatgaatatttaatttctcaatctgaacgttattctattacaatgacagaaaaatctacaattttatgtgatttatctgatttagaatatttacatgaagaattccatcaatctttacaatggatggaatctttccgtgcagcaattgataaatcttgtgatggtccagaagaaattgaagcagatggtacattaattcaaggtgttttacgtattaactctaaaaaattccatgaagcattcattccatctaaacaagcatctttagaattaggtgaaggtaaattaccagcagcatgtttaaaaccagcattagattctgaaggtccagcagaaggtgaaggtcatttaggtcgttgtatgcgtcgtttacgtgatgcagttcgtcgtttacgttataaaggtggtgatacattcacaggtttagcattaacacatgttgttcgtacatgtatgcataaccaaaaagatgttgttcaaatgccatctgaaacattaaaagcagataacatgccattagaattatctggtcgtttacaaaaaacagttacaggtttattaattgaacaatcttcttgtcaaccagcatgttgtacaacatcttcttgtcaacaagcatgttgtgttccaattaaagttaaatctgaagaaaaaattaaagcagttgaaaaatctgaaaaagaaacagttattgttcgtcgtacaaaattacaaatgcaaacagcagcattagaattccgtcaacgtattattttagatttatatatggatacaggtgaagaaatttgtttccgtgttgttgatgaatctttcgttgatgcacgtagtataatcaactttgaaaaactgagtgcattaaacacatctacatgtccaccaaaccgtgttaaattagatattttcccaaaagaatgtgaatatgttaaatggttcttacatgcagatttatcttcttatttaacaccaatgaaacatatgatttctcgtgatgcagcacaaaaaatttatttagaaatgaaattcacattacgtgattgtaacaactgttctttcgaagatttcacagaatctaaatctgttttagaagatggtcgtattcattgttctaaaaaattcttagaaaacttattagaaattttcaactctcatgttgatcatggtacaggtcatcatcatcatcatcattaataa

SEQ ID NO: 595 - protein, 437 aa, artificial
  synthetic
NGVVSLVGGPCTLTSAGQLLPSILGEEPRFGKTPLAMLAATCDEYLISQSERYSITMTEKSTILCDLSDLEYLHEEFHQSLQWMESFRAAIDKSCDGPEEIEADGTLIQGVLRINSKKFHEAFIPSKQASLELGEGKLPAACLKPALDSEGPAEGEGHLGRCMRRLRDAVRRLRYKGGDTFTGLALTHVVRTCMHNQKDVVQMPSETLKADNMPLELSGRLQKTVTGLLIEQSSCQPACCTTSSCQQACCVPIKVKSEEKIKAVEKSEKETVIVRRTKLQMQTAALEFRQRIILDLYMDTGEEICFRVVDESFVDARSIINFEKLSALNTSTCPPNRVKLDIFPKECEYVKWFLHADLSSYLTPMKHMISRDAAQKIYLEMKFTLRDCNNCSFEDFTESKSVLEDGRIHCSKKFLENLLEIFNSHVDHGTGHHHHHH

SEQ ID NO: 596 - DNA, 1317 bp, artificial
  synthetic
gcacgtagtataatcaactttgaaaaactgagtttctcttctaacgttacaccaaaaatgttacaaattttcttatctgaaaaaaaatctatttctacatatcattctacatatttagttaaccaatctgcattcgatcaacgtgttgcaacagttcatttaacattaaacaacgatcaagttttcccaattaacccaccaaaattcgataaaattgaagatcaagcatgtacatctggtggttgtttacatattttaccaattacagttacaacagcacaagcatctacagcagattatgcaatgttcaaagttgaaccagaagcagataaatatcgtttaacatatcaacaacgtttacaagaagaattagatcataaattaggtccaggtgcatcttcttctcgtgttttctctgatttagataacatggcatctcgtggtcatgatcatttagataaccatcgtgaatctatttatattccagatgatattccaattccaacagaattcgaattacgtgaatctaacatgccaggtgaattaattcgtacacaaccacaacgtttcccacaattacaaacatctgcacaagttccaccaggtcaacgttgggtttggatgaacttccaatctccatctcattctccaggtttacgttctccatctcgtccatctgatgcagcagatgaagttcgtgatacatctattaacgaatctcgtaacgaaatgaacaaacaaatttctttattaacattcgcagttaaccatttaaaagcaaacgttaaaggtgttgatcaacaagatggtaaaaaccattctggttctccaggttgtgaagaatctgatgcattccgttatggtcatacattaattcaaccattaatgttccgtttagataaccgttatcaaccaattcgtaacgcagttgaaatgatgattgcatggttagatacacaattaaaaaacaaattaattggtttagaaaaaacagcatctatttctatttctgatccattcttccgttctgcacaacgttggcgtttaacatatccaggtattaaagttgttgttgttattgatggtaactctgaagatgatttaaaacaaccaccacatcaagttgaagttgaagttgcagaacaaaacgtttctcgtatttgtcatgcattaaacacatctacatgtccaccaaaccgtgttaaattagatattttcccaaaagaatgtgcaggtgaagcatctcgtttagcacattattctaaacgttctacaattacatctcgtgaaattcatcatcatcatcatcattaataa

SEQ ID NO: 597 - protein, 437 aa, artificial
  synthetic
ARSIINFEKLSFSSNVTPKMLQIFLSEKKSISTYHSTYLVNQSAFDQRVATVHLTLNNDQVFPINPPKFDKIEDQACTSGGCLHILPITVTTAQASTADYAMFKVEPEADKYRLTYQQRLQEELDHKLGPGASSSRVFSDLDNMASRGHDHLDNHRESIYIPDDIPIPTEFELRESNMPGELIRTQPQRFPQLQTSAQVPPGQRWVWMNFQSPSHSPGLRSPSRPSDAADEVRDTSINESRNEMNKQISLLTFAVNHLKANVKGVDQQDGKNHSGSPGCEESDAFRYGHTLIQPLMFRLDNRYQPIRNAVEMMIAWLDTQLKNKLIGLEKTASISISDPFFRSAQRWRLTYPGIKVVVVIDGNSEDDLKQPPHQVEVEVAEQNVSRICHALNTSTCPPNRVKLDIFPKECAGEASRLAHYSKRSTITSREIHHHHHH

SEQ ID NO: 598 - DNA, 1317 bp, artificial
  synthetic
ttctcttctaacgttacaccaaaaatgttacaaattttcttatctgaaaaaaaatctatttctacatatcattctacatatttagttaaccaatctgcattcgatcaacgtgttgcaacagttcatttaacattaaacaacgatcaagttttcccaattaacccaccaaaattcgataaaattgaagatcaagcatgtacatctggtggttgtttacatattttaccaattacagttacaacagcacaagcatctacagcagattatgcaatgttcaaagttgaaccagaagcagataaatatcgtttaacatatcaacaacgtttacaagaagaattagatcataaattaggtccaggtgcatcttcttctcgtgttttctctgatttagataacatggcatctcgtggtcatgatcatttagataaccatcgtgaatctatttatattccagatgatattccaattccaacagaattcgaattacgtgaatctaacatgccaggtgaattaattcgtacacaaccacaacgtttcccacaattacaaacatctgcacaagttccaccaggtcaacgttgggtttggatgaacttccaatctccatctcattctccaggtttacgttctgcacgtagtataatcaactttgaaaaactgagtccatctcgtccatctgatgcagcagatgaagttcgtgatacatctattaacgaatctcgtaacgaaatgaacaaacaaatttctttattaacattcgcagttaaccatttaaaagcaaacgttaaaggtgttgatcaacaagatggtaaaaaccattctggttctccaggttgtgaagaatctgatgcattccgttatggtcatacattaattcaaccattaatgttccgtttagataaccgttatcaaccaattcgtaacgcagttgaaatgatgattgcatggttagatacacaattaaaaaacaaattaattggtttagaaaaaacagcatctatttctatttctgatccattcttccgttctgcacaacgttggcgtttaacatatccaggtattaaagttgttgttgttattgatggtaactctgaagatgatttaaaacaaccaccacatcaagttgaagttgaagttgcagaacaaaacgtttctcgtatttgtcatgcattaaacacatctacatgtccaccaaaccgtgttaaattagatattttcccaaaagaatgtgcaggtgaagcatctcgtttagcacattattctaaacgttctacaattacatctcgtgaaattcatcatcatcatcatcattaataa

SEQ ID NO: 599 - protein, 437 aa, artificial
  synthetic
FSSNVTPKMLQIFLSEKKSISTYHSTYLVNQSAFDQRVATVHLTLNNDQVFPINPPKFDKIEDQACTSGGCLHILPITVTTAQASTADYAMFKVEPEADKYRLTYQQRLQEELDHKLGPGASSSRVFSDLDNMASRGHDHLDNHRESIYIPDDIPIPTEFELRESNMPGELIRTQPQRFPQLQTSAQVPPGQRWVWMNFQSPSHSPGLRSARSIINFEKLSPSRPSDAADEVRDTSINESRNEMNKQISLLTFAVNHLKANVKGVDQQDGKNHSGSPGCEESDAFRYGHTLIQPLMFRLDNRYQPIRNAVEMMIAWLDTQLKNKLIGLEKTASISISDPFFRSAQRWRLTYPGIKVVVVIDGNSEDDLKQPPHQVEVEVAEQNVSRICHALNTSTCPPNRVKLDIFPKECAGEASRLAHYSKRSTITSREIHHHHHH

SEQ ID NO: 600 - DNA, 1317 bp, artificial
  synthetic
ggtaacatggcagttattcattctcatttattaaaaggtttacaagaaaaattcttaaaaggtgcaccagaagttattaaagcatctacattctgtaaaggttctgatgtttggtctttcggtgttgcattccatgcattcgcacaaccattcgcattaccacattctgcaatgttattcgttcaagcattatctgtttctcgtccagaacgtccaccacaatctgcagcattaggtggtttaggtccaggtgttgcattagaattaaacggtggtttatgtgttgaagcatgtggtgcattagaaggtttatggaaaattttacaattatgtggtgaattatatcatttagatgcatcttctttacaattaaaagttccaaaattattacgtgaattagttccacaatatttaattcgtcaagtttctccagatgattggcatgcattaatgacatattctacagcacgtatgtctttcgtttgtccacgtttctatcgtaacccaggtcaacgttgggtttggatgaacttccaatctccatctcattctccaggtttacgttctgttccaaacttacattggttagttgttgaaaacgcaccacgtcgtacaccattaacagcacgtaaattatcttctaaccgtgaacgtcatgttacaatgtgtgaagatttagaattacgtcgtgatttattagcagttacaggtggttgtttcatggaattatgtaaagttgcaaaatctgttgaaatgcaatcttcttctcgtgcaaacttcgttattgcattaattaacattccagcagttgcattaggtggttatcatgataacggtatgttctctccaattggtgaatcttgtgaagatattgatgaatgtggtatttctacacgtttaccaggtccaggtaaaccagttccaggtccagcaggtccagcacaagcacgtagtataatcaactttgaaaaactgagtattttaacacaattacatcaagatatgcaacaattagaaattccacaagcatatcgtggtgcattaattttacaaggtaaccgtattcgttctgttacaaaaaaagcattcacaggtttagatgcaatgaaatatggtgcagatccatctttaattaacggtgaaggttgttcttgtattcatttagcaggtttcaacgaaaaccgtcaaaaacgtgcagttttagcagcacaattcgaagcaacatctccaatgccaacaattaacgaagatacaccaatgcgtcaacgtggttctcaatcttctggttctgatcatcatcatcatcatcattaataa

SEQ ID NO: 601 - protein, 437 aa, artificial
  synthetic
GNMAVIHSHLLKGLQEKFLKGAPEVIKASTFCKGSDVWSFGVAFHAFAQPFALPHSAMLFVQALSVSRPERPPQSAALGGLGPGVALELNGGLCVEACGALEGLWKILQLCGELYHLDASSLQLKVPKLLRELVPQYLIRQVSPDDWHALMTYSTARMSFVCPRFYRNPGQRWVWMNFQSPSHSPGLRSVPNLHWLVVENAPRRTPLTARKLSSNRERHVTMCEDLELRRDLLAVTGGCFMELCKVAKSVEMQSSSRANFVIALINIPAVALGGYHDNGMFSPIGESCEDIDECGISTRLPGPGKPVPGPAGPAQARSIINFEKLSILTQLHQDMQQLEIPQAYRGALILQGNRIRSVTKKAFTGLDAMKYGADPSLINGEGCSCIHLAGFNENRQKRAVLAAQFEATSPMPTINEDTPMRQRGSQSSGSDHHHHHH

SEQ ID NO: 602 - DNA, 1317 bp, artificial
  synthetic
ggtaacatggcagttattcattctcatttattaaaaggtttacaagaaaaattcttaaaaggtgcaccagaagttattaaagcatctacattctgtaaaggttctgatgtttggtctttcggtgttgcattccatgcattcgcacaaccattcgcattaccacattctgcaatgttattcgttcaagcattatctgtttctcgtccagaacgtccaccacaatctgcagcattaggtggtttaggtccaggtgttgcattagaattaaacggtggtttatgtgttgaagcatgtggtgcattagaaggtttatggaaaattttacaattatgtggtgaattatatcatttagatgcatcttctttacaattaaaagttccaaaattattacgtgaattagttccacaatatttaattcgtcaagtttctccagatgattggcatgcattaatgacatattctacagcacgtatgtctttcgtttgtccacgtttctatcgtaacccaggtcaacgttgggtttggatgaacttccaatctccatctcattctccaggtttacgttctgttccaaacttacattggttagttgttgaaaacgcaccacgtcgtacaccattaacagcacgtgcacgtagtataatcaactttgaaaaactgagtaaattatcttctaaccgtgaacgtcatgttacaatgtgtgaagatttagaattacgtcgtgatttattagcagttacaggtggttgtttcatggaattatgtaaagttgcaaaatctgttgaaatgcaatcttcttctcgtgcaaacttcgttattgcattaattaacattccagcagttgcattaggtggttatcatgataacggtatgttctctccaattggtgaatcttgtgaagatattgatgaatgtggtatttctacacgtttaccaggtccaggtaaaccagttccaggtccagcaggtccagcacaaattttaacacaattacatcaagatatgcaacaattagaaattccacaagcatatcgtggtgcattaattttacaaggtaaccgtattcgttctgttacaaaaaaagcattcacaggtttagatgcaatgaaatatggtgcagatccatctttaattaacggtgaaggttgttcttgtattcatttagcaggtttcaacgaaaaccgtcaaaaacgtgcagttttagcagcacaattcgaagcaacatctccaatgccaacaattaacgaagatacaccaatgcgtcaacgtggttctcaatcttctggttctgatcatcatcatcatcatcattaataa

SEQ ID NO: 603 - protein, 437 aa, artificial
  synthetic
GNMAVIHSHLLKGLQEKFLKGAPEVIKASTFCKGSDVWSFGVAFHAFAQPFALPHSAMLFVQALSVSRPERPPQSAALGGLGPGVALELNGGLCVEACGALEGLWKILQLCGELYHLDASSLQLKVPKLLRELVPQYLIRQVSPDDWHALMTYSTARMSFVCPRFYRNPGQRWVWMNFQSPSHSPGLRSVPNLHWLVVENAPRRTPLTARARSIINFEKLSKLSSNRERHVTMCEDLELRRDLLAVTGGCFMELCKVAKSVEMQSSSRANFVIALINIPAVALGGYHDNGMFSPIGESCEDIDECGISTRLPGPGKPVPGPAGPAQILTQLHQDMQQLEIPQAYRGALILQGNRIRSVTKKAFTGLDAMKYGADPSLINGEGCSCIHLAGFNENRQKRAVLAAQFEATSPMPTINEDTPMRQRGSQSSGSDHHHHHH

SEQ ID NO: 604 - DNA, 1317 bp, artificial
  synthetic
gcacgtagtataatcaactttgaaaaactgagtggtaacatggcagttattcattctcatttattaaaaggtttacaagaaaaattcttaaaaggtgcaccagaagttattaaagcatctacattctgtaaaggttctgatgtttggtctttcggtgttgcattccatgcattcgcacaaccattcgcattaccacattctgcaatgttattcgttcaagcattatctgtttctcgtccagaacgtccaccacaatctgcagcattaggtggtttaggtccaggtgttgcattagaattaaacggtggtttatgtgttgaagcatgtggtgcattagaaggtttatggaaaattttacaattatgtggtgaattatatcatttagatgcatcttctttacaattaaaagttccaaaattattacgtgaattagttccacaatatttaattcgtcaagtttctccagatgattggcatgcattaatgacatattctacagcacgtatgtctttcgtttgtccacgtttctatcgtaacccaggtcaacgttgggtttggatgaacttccaatctccatctcattctccaggtttacgttctgttccaaacttacattggttagttgttgaaaacgcaccacgtcgtacaccattaacagcacgtaaattatcttctaaccgtgaacgtcatgttacaatgtgtgaagatttagaattacgtcgtgatttattagcagttacaggtggttgtttcatggaattatgtaaagttgcaaaatctgttgaaatgcaatcttcttctcgtgcaaacttcgttattgcattaattaacattccagcagttgcattaggtggttatcatgataacggtatgttctctccaattggtgaatcttgtgaagatattgatgaatgtggtatttctacacgtttaccaggtccaggtaaaccagttccaggtccagcaggtccagcacaaattttaacacaattacatcaagatatgcaacaattagaaattccacaagcatatcgtggtgcattaattttacaaggtaaccgtattcgttctgttacaaaaaaagcattcacaggtttagatgcaatgaaatatggtgcagatccatctttaattaacggtgaaggttgttcttgtattcatttagcaggtttcaacgaaaaccgtcaaaaacgtgcagttttagcagcacaattcgaagcaacatctccaatgccaacaattaacgaagatacaccaatgcgtcaacgtggttctcaatcttctggttctgatcatcatcatcatcatcattaataa

SEQ ID NO: 605 - protein, 437 aa, artificial
  synthetic
ARSIINFEKLSGNMAVIHSHLLKGLQEKFLKGAPEVIKASTFCKGSDVWSFGVAFHAFAQPFALPHSAMLFVQALSVSRPERPPQSAALGGLGPGVALELNGGLCVEACGALEGLWKILQLCGELYHLDASSLQLKVPKLLRELVPQYLIRQVSPDDWHALMTYSTARMSFVCPRFYRNPGQRWVWMNFQSPSHSPGLRSVPNLHWLVVENAPRRTPLTARKLSSNRERHVTMCEDLELRRDLLAVTGGCFMELCKVAKSVEMQSSSRANFVIALINIPAVALGGYHDNGMFSPIGESCEDIDECGISTRLPGPGKPVPGPAGPAQILTQLHQDMQQLEIPQAYRGALILQGNRIRSVTKKAFTGLDAMKYGADPSLINGEGCSCIHLAGFNENRQKRAVLAAQFEATSPMPTINEDTPMRQRGSQSSGSDHHHHHH

SEQ ID NO: 606 - DNA, 1317 bp, artificial
  synthetic
ttctcttctaacgttacaccaaaaatgttacaaattttcttatctgaaaaaaaatctatttctacatatcattctacatatttagttaaccaatctgcattcgatcaacgtgttgcaacagttcatttaacattaaacaacgatcaagttttcccaattaacccaccaaaattcgataaaattgaagatcaagcatgtacatctggtggttgtttacatattttaccaattacagttacaacagcacaagcatctacagcagattatgcaatgttcaaagttgaaccagaagcagataaatatcgtttaacatatcaacaacgtttacaagaagaattagatcataaattaggtccaggtgcatcttcttctcgtgttttctctgatttagataacatggcatctcgtggtcatgatcatttagataaccatcgtgaatctatttatattccagatgatattccaattccaacagaattcgaattacgtgaatctaacatgccaggtgaattaattcgtacacaaccacaacgtttcccacaattacaaacatctgcacaagttccaccaggtcaacgttgggtttggatgaacttccaatctccatctcattctccaggtttacgttctccatctcgtccatctgatgcagcagatgaagttcgtgatacatctattaacgaatctcgtaacgaaatgaacaaacaaatttctttattaacattcgcagttaaccatttaaaagcaaacgttaaaggtgttgatcaacaagatggtaaaaaccattctggttctccaggttgtgaagaatctgatgcattccgttatggtcatacattaattcaaccattaatgttccgtttagataaccgttatcaaccaattcgtaacgcagttgaaatgatgattgcatggttagatacacaattaaaaaacaaattaattgcacgtagtataatcaactttgaaaaactgagtggtttagaaaaaacagcatctatttctatttctgatccattcttccgttctgcacaacgttggcgtttaacatatccaggtattaaagttgttgttgttattgatggtaactctgaagatgatttaaaacaaccaccacatcaagttgaagttgaagttgcagaacaaaacgtttctcgtatttgtcatgcattaaacacatctacatgtccaccaaaccgtgttaaattagatattttcccaaaagaatgtgcaggtgaagcatctcgtttagcacattattctaaacgttctacaattacatctcgtgaaattcatcatcatcatcatcattaataa

SEQ ID NO: 607 - protein, 437 aa, artificial
  synthetic
FSSNVTPKMLQIFLSEKKSISTYHSTYLVNQSAFDQRVATVHLTLNNDQVFPINPPKFDKIEDQACTSGGCLHILPITVTTAQASTADYAMFKVEPEADKYRLTYQQRLQEELDHKLGPGASSSRVFSDLDNMASRGHDHLDNHRESIYIPDDIPIPTEFELRESNMPGELIRTQPQRFPQLQTSAQVPPGQRWVWMNFQSPSHSPGLRSPSRPSDAADEVRDTSINESRNEMNKQISLLTFAVNHLKANVKGVDQQDGKNHSGSPGCEESDAFRYGHTLIQPLMFRLDNRYQPIRNAVEMMIAWLDTQLKNKLIARSIINFEKLSGLEKTASISISDPFFRSAQRWRLTYPGIKVVVVIDGNSEDDLKQPPHQVEVEVAEQNVSRICHALNTSTCPPNRVKLDIFPKECAGEASRLAHYSKRSTITSREIHHHHHH

SEQ ID NO: 608 - DNA, 1317 bp, artificial; identical to SEQ ID NO: 604
  synthetic

SEQ ID NO: 609 - protein, 437 aa, artificial; identical to SEQ ID NO: 605
  synthetic

SEQ ID NO: 610 - DNA, 1317 bp, artificial
  synthetic
aacggtgttgtttctttagttggtggtccatgtacattaacatctgcaggtcaattattaccatctattttaggtgaagaaccacgtttcggtaaaacaccattagcaatgttagcagcaacatgtgatgaatatttaatttctcaatctgaacgttattctattacaatgacagaaaaatctacaattttatgtgatttatctgatttagaatatttacatgaagaattccatcaatctttacaatggatggaatctttccgtgcagcaattgataaatcttgtgatggtccagaagaaattgaagcagatggtgcacgtagtataatcaactttgaaaaactgagtacattaattcaaggtgttttacgtattaactctaaaaaattccatgaagcattcattccatctaaacaagcatctttagaattaggtgaaggtaaattaccagcagcatgtttaaaaccagcattagattctgaaggtccagcagaaggtgaaggtcatttaggtcgttgtatgcgtcgtttacgtgatgcagttcgtcgtttacgttataaaggtggtgatacattcacaggtttagcattaacacatgttgttcgtacatgtatgcataaccaaaaagatgttgttcaaatgccatctgaaacattaaaagcagataacatgccattagaattatctggtcgtttacaaaaaacagttacaggtttattaattgaacaatcttcttgtcaaccagcatgttgtacaacatcttcttgtcaacaagcatgttgtgttccaattaaagttaaatctgaagaaaaaattaaagcagttgaaaaatctgaaaaagaaacagttattgttcgtcgtacaaaattacaaatgcaaacagcagcattagaattccgtcaacgtattattttagatttatatatggatacaggtgaagaaatttgtttccgtgttgttgatgaatctttcgttgatgcattaaacacatctacatgtccaccaaaccgtgttaaattagatattttcccaaaagaatgtgaatatgttaaatggttcttacatgcagatttatcttcttatttaacaccaatgaaacatatgatttctcgtgatgcagcacaaaaaatttatttagaaatgaaattcacattacgtgattgtaacaactgttctttcgaagatttcacagaatctaaatctgttttagaagatggtcgtattcattgttctaaaaaattcttagaaaacttattagaaattttcaactctcatgttgatcatggtacaggtcatcatcatcatcatcattaataa

SEQ ID NO: 611 - protein, 437 aa, artificial
  synthetic
NGVVSLVGGPCTLTSAGQLLPSILGEEPRFGKTPLAMLAATCDEYLISQSERYSITMTEKSTILCDLSDLEYLHEEFHQSLQWMESFRAAIDKSCDGPEEIEADGARSIINFEKLSTLIQGVLRINSKKFHEAFIPSKQASLELGEGKLPAACLKPALDSEGPAEGEGHLGRCMRRLRDAVRRLRYKGGDTFTGLALTHVVRTCMHNQKDVVQMPSETLKADNMPLELSGRLQKTVTGLLIEQSSCQPACCTTSSCQQACCVPIKVKSEEKIKAVEKSEKETVIVRRTKLQMQTAALEFRQRIILDLYMDTGEEICFRVVDESFVDALNTSTCPPNRVKLDIFPKECEYVKWFLHADLSSYLTPMKHMISRDAAQKIYLEMKFTLRDCNNCSFEDFTESKSVLEDGRIHCSKKFLENLLEIFNSHVDHGTGHHHHHH

SEQ ID NO: 612 - DNA, 1317 bp, artificial
  synthetic
aacggtgttgtttctttagttggtggtccatgtacattaacatctgcaggtcaattattaccatctattttaggtgaagaaccacgtttcggtaaaacaccattagcaatgttagcagcaacatgtgatgaatatttaatttctcaatctgaacgttattctattacaatgacagaaaaatctacaattttatgtgatttatctgatttagaatatttacatgaagaattccatcaatctttacaatggatggaatctttccgtgcagcaattgataaatcttgtgatggtccagaagaaattgaagcagatggtacattaattcaaggtgttttacgtattaactctaaaaaattccatgaagcattcattccatctaaacaagcatctttagaattaggtgaaggtaaattaccagcagcatgtttaaaaccagcattagattctgaaggtccagcagaaggtgaaggtcatttaggtcgttgtatgcgtcgtttacgtgatgcagttcgtcgtttacgttataaaggtggtgatacattcacaggtttagcattaacacatgttgttcgtacatgtatgcataaccaaaaagatgttgttcaaatgccatctgaaacattaaaagcagcacgtagtataatcaactttgaaaaactgagtgataacatgccattagaattatctggtcgtttacaaaaaacagttacaggtttattaattgaacaatcttcttgtcaaccagcatgttgtacaacatcttcttgtcaacaagcatgttgtgttccaattaaagttaaatctgaagaaaaaattaaagcagttgaaaaatctgaaaaagaaacagttattgttcgtcgtacaaaattacaaatgcaaacagcagcattagaattccgtcaacgtattattttagatttatatatggatacaggtgaagaaatttgtttccgtgttgttgatgaatctttcgttgatgcattaaacacatctacatgtccaccaaaccgtgttaaattagatattttcccaaaagaatgtgaatatgttaaatggttcttacatgcagatttatcttcttatttaacaccaatgaaacatatgatttctcgtgatgcagcacaaaaaatttatttagaaatgaaattcacattacgtgattgtaacaactgttctttcgaagatttcacagaatctaaatctgttttagaagatggtcgtattcattgttctaaaaaattcttagaaaacttattagaaattttcaactctcatgttgatcatggtacaggtcatcatcatcatcatcattaataa

SEQ ID NO: 613 - protein, 437 aa, artificial
  synthetic
NGVVSLVGGPCTLTSAGQLLPSILGEEPRFGKTPLAMLAATCDEYLISQSERYSITMTEKSTILCDLSDLEYLHEEFHQSLQWMESFRAAIDKSCDGPEEIEADGTLIQGVLRINSKKFHEAFIPSKQASLELGEGKLPAACLKPALDSEGPAEGEGHLGRCMRRLRDAVRRLRYKGGDTFTGLALTHVVRTCMHNQKDVVQMPSETLKAARSIINFEKLSDNMPLELSGRLQKTVTGLLIEQSSCQPACCTTSSCQQACCVPIKVKSEEKIKAVEKSEKETVIVRRTKLQMQTAALEFRQRIILDLYMDTGEEICFRVVDESFVDALNTSTCPPNRVKLDIFPKECEYVKWFLHADLSSYLTPMKHMISRDAAQKIYLEMKFTLRDCNNCSFEDFTESKSVLEDGRIHCSKKFLENLLEIFNSHVDHGTGHHHHHH

SEQ ID NO: 614 - DNA, 1317 bp, artificial; identical to SEQ ID NO: 584
  synthetic

SEQ ID NO: 615 - protein, 437 aa, artificial; identical to SEQ ID NO: 585
  synthetic

SEQ ID NO: 616 - DNA, 1317 bp, artificial; identical to SEQ ID NO: 590
  synthetic

SEQ ID NO: 617 - protein, 437 aa, artificial; identical to SEQ ID NO: 591
  synthetic

SEQ ID NO: 618 - protein, 437 aa, artificial
  synthetic
FVTRVVEEIKNMKIGDPLDRSNLLLLGATAIQDRLQAGVPETRSIQGPSLSSRRNVMSEASLDLNIPAPLHPEKTVNMTYQPTTLKNGRLTVGQNIILHGAELVGHAEALLKAAIQYGTEEDVVKSYLKQWRSYNICIWDTAGREQFTGIVSWGQGCTTVGHFGVYTRKSRDAARSRRRKENFEFYELAMRGKPKVIDLKRNEATVETLTETHKVAAPDVIVPTLDTVRHECLAEAAVAAVSVADTVRECPPAILLLLQLWDCGAQETDNERSYVRAFLKHSAKLVYMLESKHLVRGQCDRRRHDTAATEIGGKIFQHIADYLPSNARPRFLRIQDSNEVGEGPFSSSVELAVLPKEVKADPAALMALLHLLPSPTPNIEVEKMRRAETSARVENSPQVTAYRFLEEMRWRLPLANMSYYARSIINFEKLSHHHHHH

SEQ ID NO: 619 - DNA, 1317 bp, artificial
  synthetic
ttctcttctaacgttacaccaaaaatgttacaaattttcttatctgaaaaaaaatctatttctacatatcattctacatatttagttaaccaatctgcattcgatcaacgtgttgcaacagttcatttaacattaaacaacgatcaagttttcccaattaacccaccaaaattcgataaaattgaagatcaagcatgtacatctggtggttgtttacatattttaccaattacagttacaacagcacaagcatctacagcagattatgcaatgttcaaagttgaaccagaagcagataaatatcgtttaacatatgcacgtagtataatcaactttgaaaaactgagtcaacaacgtttacaagaagaattagatcataaattaggtccaggtgcatcttcttctcgtgttttctctgatttagataacatggcatctcgtggtcatgatcatttagataaccatcgtgaatctatttatattccagatgatattccaattccaacagaattcgaattacgtgaatctaacatgccaggtgaattaattcgtacacaaccacaacgtttcccacaattacaaacatctgcacaagttccaccaggtcaacgttgggtttggatgaacttccaatctccatctcattctccaggtttacgttctccatctcgtccatctgatgcagcagatgaagttcgtgatacatctattaacgaatctcgtaacgaaatgaacaaacaaatttctttattaacattcgcagttaaccatttaaaagcaaacgttaaaggtgttgatcaacaagatggtaaaaaccattctggttctccaggttgtgaagaatctgatgcattccgttatggtcatacattaattcaaccattaatgttccgtttagataaccgttatcaaccaattcgtaacgcagttgaaatgatgattgcatggttagatacacaattaaaaaacaaattaattggtttagaaaaaacagcatctatttctatttctgatccattcttccgttctgcacaacgttggcgtttaacatatccaggtattaaagttgttgttgttattgatggtaactctgaagatgatttaaaacaaccaccacatcaagttgaagttgaagttgcagaacaaaacgtttctcgtatttgtcatgcattaaacacatctacatgtccaccaaaccgtgttaaattagatattttcccaaaagaatgtgcaggtgaagcatctcgtttagcacattattctaaacgttctacaattacatctcgtgaaattcatcatcatcatcatcattaataa

SEQ ID NO: 620 - protein, 437 aa, artificial
  synthetic
FSSNVTPKMLQIFLSEKKSISTYHSTYLVNQSAFDQRVATVHLTLNNDQVFPINPPKFDKIEDQACTSGGCLHILPITVTTAQASTADYAMFKVEPEADKYRLTYARSIINFEKLSQQRLQEELDHKLGPGASSSRVFSDLDNMASRGHDHLDNHRESIYIPDDIPIPTEFELRESNMPGELIRTQPQRFPQLQTSAQVPPGQRWVWMNFQSPSHSPGLRSPSRPSDAADEVRDTSINESRNEMNKQISLLTFAVNHLKANVKGVDQQDGKNHSGSPGCEESDAFRYGHTLIQPLMFRLDNRYQPIRNAVEMMIAWLDTQLKNKLIGLEKTASISISDPFFRSAQRWRLTYPGIKVVVVIDGNSEDDLKQPPHQVEVEVAEQNVSRICHALNTSTCPPNRVKLDIFPKECAGEASRLAHYSKRSTITSREIHHHHHH

SEQ ID NO: 621 - DNA, 1317 bp, artificial
  synthetic
caatcttcttctcgtgcaaacttcgttattgcattaattaacattccagcagttgcattaggtgtttctaacttagaagaattaccattaaaccaatatcgtgaatatattaaagttcaaacagatttaaaaaacggtcgtttaacagttggtcaaaacattattttacatggtgcagaattagttggtttaattttacaaggtaaccgtattcgttctgttacaaaaaaagcattcacaggtttagatgcattagaagaattatatgcattattcaaagcacaacatttaacatcttgttattggggtggttctatgttaattccattctgtgaagaattaaacttcaaaacaacatggttattccataaaacacgtaaccaattaaaagaaatttcttcttctacagattctcaaaaaaaagaagaaacaaaaggtaaatctcgtttcaaaggtaaacattctttagatggtgatgaagaagaagatgatgatgatggtggttctaacgaagttggtgaaggtccattctcttcttctgttgaattagcagttttaccaaaagaaatggcattcgtttatttacgtgttttccgtaaagcacaaaaacaagttaaaaaaattgattctacatctaactatacaggtttcgcaaacaaagatggttctaaaccatcttctttaggtgtttctgcatggcaaaactctattcgtcataacttatgtttaaacgattgtttcgttaaaattccacgtaacttacaattcgaatatattgatgatccatgggttcaacgtattgaaccagaatggtctattgttttagtttctttagattctgttgaaccaccagattggtctttcggtgaagaagaagatggtcgtgttgattatatttctacacgtcgtgcatctcaacataaatgtgcattattattacaagaagcaacaaaccaagcatctacaaaatctcataaatattctaaagttacaaactctttatctttagaacgtacaacagaagaagttgcacgtaaccaacgtgcagttgaagcagaaaaatgtgcacgtgcagcaccattcaaagcaccagaattattaaaaggtcaatctgaagatgaacaaccagatgcaccattaccagcacaagcacaatctgcatgggcaccagcaccagcaccagcaacatatcgtgcagttgcaccaccacaaggtgttgttaaagcatcttattctttatctgaagatgcagaaggtgaagcacgtagtataatcaactttgaaaaactgagtcatcatcatcatcatcattaataa

SEQ ID NO: 622 - protein, 437 aa, artificial
  synthetic
QSSSRANFVIALINIPAVALGVSNLEELPLNQYREYIKVQTDLKNGRLTVGQNIILHGAELVGLILQGNRIRSVTKKAFTGLDALEELYALFKAQHLTSCYWGGSMLIPFCEELNFKTTWLFHKTRNQLKEISSSTDSQKKEETKGKSRFKGKHSLDGDEEEDDDDGGSNEVGEGPFSSSVELAVLPKEMAFVYLRVFRKAQKQVKKIDSTSNYTGFANKDGSKPSSLGVSAWQNSIRHNLCLNDCFVKIPRNLQFEYIDDPWVQRIEPEWSIVLVSLDSVEPPDWSFGEEEDGRVDYISTRRASQHKCALLLQEATNQASTKSHKYSKVTNSLSLERTTEEVARNQRAVEAEKCARAAPFKAPELLKGQSEDEQPDAPLPAQAQSAWAPAPAPATYRAVAPPQGVVKASYSLSEDAEGEARSIINFEKLSHHHHHH

SEQ ID NO: 623 - DNA, 372 bp, artificial
  synthetic
acaacatctaaagattggcatcatggtggtccagaaaacattttattatgtacagattgtcgtcgttataacccatctttaaaaggtttaaacgttacaggttatgaaacacaagcaggtgaattcgtttctcgtgcagcagttcattgtgcattatcttctccaccaccagaagcacgtgcattagcagttcaattacgtgatggtgattctaactgtcaatggtctgttttatatgttattattgcaacagaaatgaacaaacaaatttctttattaacattcgcagttaaccatttaaaagcaaacgttaaagcacgtagtataatcaactttgaaaaactgagtcatcatcatcatcatcattaataa

SEQ ID NO: 624 - protein, 122 aa, artificial
  synthetic
TTSKDWHHGGPENILLCTDCRRYNPSLKGLNVTGYETQAGEFVSRAAVHCALSSPPPEARALAVQLRDGDSNCQWSVLYVIIATEMNKQISLLTFAVNHLKANVKARSIINFEKLSHHHHHH

SEQ ID NO: 625 - DNA, 372 bp, artificial
  synthetic
gcatggcaaaactctattcgtcataacttatgtttaaacgattgtttcgttaaaattccacgtgatggtttaccaggtccaccaggtacaatgatgatgttaccattccgttatggtggtgatggtattacagttcatacaccattattaccatctgcaacaggtttattaaacgataacacattcgcagcaggtgaagcatctcgtttagcacattattctaaacgttctacaattacatctcgtgaaattggtgaagttttaccaacaccaggtcaagaaccaccaacaccaattcatgatttatctgcatctgcacgtagtataatcaactttgaaaaactgagtcatcatcatcatcatcattaataa

SEQ ID NO: 626 - protein, 122 aa, artificial
  synthetic
AWQNSIRHNLCLNDCFVKIPRDGLPGPPGTMMMLPFRYGGDGITVHTPLLPSATGLLNDNTFAAGEASRLAHYSKRSTITSREIGEVLPTPGQEPPTPIHDLSASARSIINFEKLSHHHHHH

SEQ ID NO: 627 - DNA, 372 bp, artificial
  synthetic
gaacgtacaacagaagaagttgcacgtaaccaacgtgcagttgaagcagaaaaatgtgcacgttctttcccagcacaagatccatgtacaacaacatatgttgcagcaacagaaccagttccagaaccatctgttccattaggtggtgcaccatcttgtggtatgttaatggataaaccacatccaccatctttatctgttaaaattcgtgttatgccatgttctttcttcttattattacgtttcttcttatctaaacaacaagttgcagatgaagatgaaatgtggaaacaacgtcgtcgtcaacaatctgaagcacgtagtataatcaactttgaaaaactgagtcatcatcatcatcatcattaataa

SEQ ID NO: 628 - protein, 122 aa, artificial
  synthetic
ERTTEEVARNQRAVEAEKCARSFPAQDPCTTTYVAATEPVPEPSVPLGGAPSCGMLMDKPHPPSLSVKIRVMPCSFFLLLRFFLSKQQVADEDEMWKQRRRQQSEARSIINFEKLSHHHHHH

SEQ ID NO: 629 - DNA, 687 bp, artificial
  synthetic
ccatctgttccattaggtggtgcaccatcttgtggtatgttaatggataaaccacatccaccagaacgtacaacagaagaagttgcacgtaaccaacgtgcagttgaagcagaaaaatgtgcacgtgcaggtgaagcatctcgtttagcacattattctaaacgttctacaattacatctcgtgaaattgatggtttaccaggtccaccaggtacaatgatgatgttaccattccgttatggtggtgatggtaacttacaattcgaatatattgatgatccatgggttcaacgtattgaaccagaatggtctattgaaatgaacaaacaaatttctttattaacattcgcagttaaccatttaaaagcaaacgttaaacgttataacccatctttaaaaggtttaaacgttacaggttatgaaacacaagcaggtgaattcgttcaattacgtgatggtgattctaactgtcaatggtctgttttatatgttattattgcaacagtttctcgtgcagcagttcattgtgcattatcttctccaccaccagaagcacgtgcattagcatctttcccagcacaagatccatgtacaacaacatatgttgcagcaacagaaccagttccagaagcacgtagtataatcaactttgaaaaactgagtcatcatcatcatcatcattaataa

SEQ ID NO: 630 - protein, 227 aa, artificial
  synthetic
PSVPLGGAPSCGMLMDKPHPPERTTEEVARNQRAVEAEKCARAGEASRLAHYSKRSTITSREIDGLPGPPGTMMMLPFRYGGDGNLQFEYIDDPWVQRIEPEWSIEMNKQISLLTFAVNHLKANVKRYNPSLKGLNVTGYETQAGEFVQLRDGDSNCQWSVLYVIIATVSRAAVHCALSSPPPEARALASFPAQDPCTTTYVAATEPVPEARSIINFEKLSHHHHHH

SEQ ID NO: 631 - DNA, 372 bp, artificial
  synthetic
ggtgaagttttaccaacaccaggtcaagaaccaccaacaccaattcatgatttatctgcatctaaacgtaaagattctcgtacagtttatcaatctcaatatacaaactggtctgttgaacaattaccaccattaccagatgtttctgcattctcttctggtcgtggtggtggtgaaggtcgtggttctaaccaaccacgtttaattcaatgtgaaaacaaagttggtaaattattattcattacagaaatttctaaacaacaagttgcagatgaagatgaaatgtggaaacaacgtcgtcgtcaacaatctgaagcacgtagtataatcaactttgaaaaactgagtcatcatcatcatcatcattaataa

SEQ ID NO: 632 - protein, 122 aa, artificial
  synthetic
GEVLPTPGQEPPTPIHDLSASKRKDSRTVYQSQYTNWSVEQLPPLPDVSAFSSGRGGGEGRGSNQPRLIQCENKVGKLLFITEISKQQVADEDEMWKQRRRQQSEARSIINFEKLSHHHHHH

SEQ ID NO: 633 - DNA, 372 bp, artificial
  synthetic
attacagttcatacaccattattaccatctgcaacaggtttattaaacgataacacattcgcagcatggcaaaactctattcgtcataacttatgtttaaacgattgtttcgttaaaattccacgtaaatctacaacaaaacgttctccaaaaccacaaaacaaaaaaaaaacaaaaaaagttattgaatctttatctgttaaaattcgtgttatgccatgttctttcttcttattattacgtttcttcttaacaacatctaaagattggcatcatggtggtccagaaaacattttattatgtacagattgtcgtgcacgtagtataatcaactttgaaaaactgagtcatcatcatcatcatcattaataa

SEQ ID NO: 634 - protein, 122 aa, artificial
  synthetic
ITVHTPLLPSATGLLNDNTFAAWQNSIRHNLCLNDCFVKIPRKSTTKRSPKPQNKKKTKKVIESLSVKIRVMPCSFFLLLRFFLTTSKDWHHGGPENILLCTDCRARSIINFEKLSHHHHHH

SEQ ID NO: 635 - DNA, 372 bp, artificial
  synthetic
ccatctgttccattaggtggtgcaccatcttgtggtatgttaatggataaaccacatccaccagaacgtacaacagaagaagttgcacgtaaccaacgtgcagttgaagcagaaaaatgtgcacgtgcaggtgaagcatctcgtttagcacattattctaaacgttctacaattacatctcgtgaaattgatggtttaccaggtccaccaggtacaatgatgatgttaccattccgttatggtggtgatggtaacttacaattcgaatatattgatgatccatgggttcaacgtattgaaccagaatggtctattgcacgtagtataatcaactttgaaaaactgagtcatcatcatcatcatcattaataa

SEQ ID NO: 636 - protein, 122 aa, artificial
  synthetic
PSVPLGGAPSCGMLMDKPHPPERTTEEVARNQRAVEAEKCARAGEASRLAHYSKRSTITSREIDGLPGPPGTMMMLPFRYGGDGNLQFEYIDDPWVQRIEPEWSIARSIINFEKLSHHHHHH

SEQ ID NO: 637 - DNA, 372 bp, artificial
  synthetic
gaaatgaacaaacaaatttctttattaacattcgcagttaaccatttaaaagcaaacgttaaacgttataacccatctttaaaaggtttaaacgttacaggttatgaaacacaagcaggtgaattcgttcaattacgtgatggtgattctaactgtcaatggtctgttttatatgttattattgcaacagtttctcgtgcagcagttcattgtgcattatcttctccaccaccagaagcacgtgcattagcatctttcccagcacaagatccatgtacaacaacatatgttgcagcaacagaaccagttccagaagcacgtagtataatcaactttgaaaaactgagtcatcatcatcatcatcattaataa

SEQ ID NO: 638 - protein, 122 aa, artificial
  synthetic
EMNKQISLLTFAVNHLKANVKRYNPSLKGLNVTGYETQAGEFVQLRDGDSNCQWSVLYVIIATVSRAAVHCALSSPPPEARALASFPAQDPCTTTYVAATEPVPEARSIINFEKLSHHHHHH

SEQ ID NO: 639 - DNA, 372 bp, artificial
  synthetic
aaatctacaacaaaacgttctccaaaaccacaaaacaaaaaaaaaacaaaaaaagttattgaagatggtttaccaggtccaccaggtacaatgatgatgttaccattccgttatggtggtgatggtccaccattaccagatgtttctgcattctcttctggtcgtggtggtggtgaaggtcgtggttctaaccaaccacgtttaattcaatgtgaaaacaaagttggtaaattattattcattacagaaattgcatggcaaaactctattcgtcataacttatgtttaaacgattgtttcgttaaaattccacgtgcacgtagtataatcaactttgaaaaactgagtcatcatcatcatcatcattaataa

SEQ ID NO: 640 - protein, 122 aa, artificial
  synthetic
KSTTKRSPKPQNKKKTKKVIEDGLPGPPGTMMMLPFRYGGDGPPLPDVSAFSSGRGGGEGRGSNQPRLIQCENKVGKLLFITEIAWQNSIRHNLCLNDCFVKIPRARSIINFEKLSHHHHHH

SEQ ID NO: 641 - DNA, 372 bp, artificial
  synthetic
aacttacaattcgaatatattgatgatccatgggttcaacgtattgaaccagaatggtctatttctttcccagcacaagatccatgtacaacaacatatgttgcagcaacagaaccagttccagaagttcaattacgtgatggtgattctaactgtcaatggtctgttttatatgttattattgcaacaattacagttcatacaccattattaccatctgcaacaggtttattaaacgataacacattcgcacgttataacccatctttaaaaggtttaaacgttacaggttatgaaacacaagcaggtgaattcgcacgtagtataatcaactttgaaaaactgagtcatcatcatcatcatcattaataa

SEQ ID NO: 642 - protein, 122 aa, artificial
  synthetic
NLQFEYIDDPWVQRIEPEWSISFPAQDPCTTTYVAATEPVPEVQLRDGDSNCQWSVLYVIIATITVHTPLLPSATGLLNDNTFARYNPSLKGLNVTGYETQAGEFARSIINFEKLSHHHHHH

SEQ ID NO: 643 - DNA, 372 bp, artificial
  synthetic
gaattaatggataaaccattaacattccgtattttatctgatttaattttcgatttcgaagatcaagcatgtacatctggtggttgtttacatattttaccaattacagttacaacagcacaagcagttgaagatgaacgtcaaatggatgatttacaaggtgcagcaccaccaattccaccacgtttaaactatacaattttaatgcgtccacgtttcgttattttattaacagttatttcttggttcttaacatctaactatacaggtttcgcaaacaaagatggttctaaaccatcttctttaggtgtttctgcacgtagtataatcaactttgaaaaactgagtcatcatcatcatcatcattaataa

SEQ ID NO: 644 - protein, 122 aa, artificial
  synthetic
ELMDKPLTFRILSDLIFDFEDQACTSGGCLHILPITVTTAQAVEDERQMDDLQGAAPPIPPRLNYTILMRPRFVILLTVISWFLTSNYTGFANKDGSKPSSLGVSARSIINFEKLSHHHHHH

SEQ ID NO: 645 - DNA, 372 bp, artificial
  synthetic
aaattaggtatttctttctctgaagatcaaaaaaaagaaaaagatcaattaggtaaagcaccagcagcaccattcaaagcaccagaattattaaaaggtcaatctgaagatgaacaaccagatgcagcaacaaaccaagcatctacaaaatctcataaatattctaaagttacaaactctttatctttagtttctaacttagaagaattaccattaaaccaatatcgtgaatatattaaagttcaaacagatgcaggttgtcaattatggttatgtacattattagcagttttaaaagttggttggttattcccagcacgtagtataatcaactttgaaaaactgagtcatcatcatcatcatcattaataa

SEQ ID NO: 646 - protein, 122 aa, artificial
  synthetic
KLGISFSEDQKKEKDQLGKAPAAPFKAPELLKGQSEDEQPDAATNQASTKSHKYSKVTNSLSLVSNLEELPLNQYREYIKVQTDAGCQLWLCTLLAVLKVGWLFPARSIINFEKLSHHHHHH

SEQ ID NO: 647 - DNA, 372 bp, artificial
  synthetic
tctcaatcttcttgttgtaaaccatgttgttgtcaatcttcttgttgtaaaccatgttgttgtttaggtaacggtacaattttatggattattgttttacaaccagcattacatcgtccaatgcatttagttttagcagtttctatgacaaaaacaaaaaacgaagtttgtggttatccagttattgttttctcttctaacgttacaccaaaaatgttacaaattttcttatctgaaaaaaaatctatttctattgttttaccatctaacgttattaacttctgtcaagcagaaaaaccagaaccaacaaaccaagcacgtagtataatcaactttgaaaaactgagtcatcatcatcatcatcattaataa

SEQ ID NO: 648 - protein, 122 aa, artificial
  synthetic
SQSSCCKPCCCQSSCCKPCCCLGNGTILWIIVLQPALHRPMHLVLAVSMTKTKNEVCGYPVIVFSSNVTPKMLQIFLSEKKSISIVLPSNVINFCQAEKPEPTNQARSIINFEKLSHHHHHH

SEQ ID NO: 649 - DNA, 1317 bp, artificial
  synthetic
ttctcttctaacgttacaccaaaaatgttacaaattttcttatctgaaaaaaaatctatttctcaagcatgtacatctggtggttgtttacatattttaccaattacagttacaacagcacaagcaacatatcattctacatatttagttaaccaatctgcattcgatcaacgtgttgcaacagttcatttaggtaacggtacaattttatggattattgttttacaaccagcattacatcgtccaatgcattctcaatcttcttgttgtaaaccatgttgttgtcaatcttcttgttgtaaaccatgttgttgtacatctaactatacaggtttcgcaaacaaagatggttctaaaccatcttctttaggtgtttctttctgtggtggtgcattaattcatccatgtcgtgttttaacagcagcacattgtacagatattatggcattcgtttatttacgtgttttccgtaaagcacaaaaacaagttaaaaaaattgattctgcaggttgtcaattatggttatgtacattattagcagttttaaaagttggttggttattcccaaactatacaattttaatgcgtccacgtttcgttattttattaacagttatttcttggttcttaccatctcgtccatctgatgcagcagatgaagttcgtgatacatctattaacgaatctcgtaacgtttctaacttagaagaattaccattaaaccaatatcgtgaatatattaaagttcaaacagatacaacaacattacgtgaatggtctacaatttgtcgtcaattatatgttcaagataaccgtgaattagttttagcagtttctatgacaaaaacaaaaaacgaagtttgtggttatccagttattgttgaattaatggataaaccattaacattccgtattttatctgatttaattttcgatttcgaagatgcaacaaaccaagcatctacaaaatctcataaatattctaaagttacaaactctttatctttaaaattaggtatttctttctctgaagatcaaaaaaaagaaaaagatcaattaggtaaagcaccaattgttttaccatctaacgttattaacttctgtcaagcagaaaaaccagaaccaacaaaccaagcagcaccattcaaagcaccagaattattaaaaggtcaatctgaagatgaacaaccagatgcagttgaagatgaacgtcaaatggatgatttacaaggtgcagcaccaccaattccaccacgtttagcacgtagtataatcaactttgaaaaactgagtcatcatcatcatcatcattaataa

SEQ ID NO: 650 - protein, 437 aa, artificial
  synthetic
FSSNVTPKMLQIFLSEKKSISQACTSGGCLHILPITVTTAQATYHSTYLVNQSAFDQRVATVHLGNGTILWIIVLQPALHRPMHSQSSCCKPCCCQSSCCKPCCCTSNYTGFANKDGSKPSSLGVSFCGGALIHPCRVLTAAHCTDIMAFVYLRVFRKAQKQVKKIDSAGCQLWLCTLLAVLKVGWLFPNYTILMRPRFVILLTVISWFLPSRPSDAADEVRDTSINESRNVSNLEELPLNQYREYIKVQTDTTTLREWSTICRQLYVQDNRELVLAVSMTKTKNEVCGYPVIVELMDKPLTFRILSDLIFDFEDATNQASTKSHKYSKVTNSLSLKLGISFSEDQKKEKDQLGKAPIVLPSNVINFCQAEKPEPTNQAAPFKAPELLKGQSEDEQPDAVEDERQMDDLQGAAPPIPPRLARSIINFEKLSHHHHHH

SEQ ID NO: 651 - DNA, 687 bp, artificial
  synthetic
ttctcttctaacgttacaccaaaaatgttacaaattttcttatctgaaaaaaaatctatttctcaagcatgtacatctggtggttgtttacatattttaccaattacagttacaacagcacaagcaacatatcattctacatatttagttaaccaatctgcattcgatcaacgtgttgcaacagttcatttaggtaacggtacaattttatggattattgttttacaaccagcattacatcgtccaatgcattctcaatcttcttgttgtaaaccatgttgttgtcaatcttcttgttgtaaaccatgttgttgtacatctaactatacaggtttcgcaaacaaagatggttctaaaccatcttctttaggtgtttctttctgtggtggtgcattaattcatccatgtcgtgttttaacagcagcacattgtacagatattatggcattcgtttatttacgtgttttccgtaaagcacaaaaacaagttaaaaaaattgattctgcaggttgtcaattatggttatgtacattattagcagttttaaaagttggttggttattcccaaactatacaattttaatgcgtccacgtttcgttattttattaacagttatttcttggttcttagcacgtagtataatcaactttgaaaaactgagtcatcatcatcatcatcattaataa

SEQ ID NO: 652 - protein, 227 aa, artificial
  synthetic
FSSNVTPKMLQIFLSEKKSISQACTSGGCLHILPITVTTAQATYHSTYLVNQSAFDQRVATVHLGNGTILWIIVLQPALHRPMHSQSSCCKPCCCQSSCCKPCCCTSNYTGFANKDGSKPSSLGVSFCGGALIHPCRVLTAAHCTDIMAFVYLRVFRKAQKQVKKIDSAGCQLWLCTLLAVLKVGWLFPNYTILMRPRFVILLTVISWFLARSIINFEKLSHHHHHH

SEQ ID NO: 653 - DNA, 687 bp, artificial
  synthetic
ccatctcgtccatctgatgcagcagatgaagttcgtgatacatctattaacgaatctcgtaacgtttctaacttagaagaattaccattaaaccaatatcgtgaatatattaaagttcaaacagatacaacaacattacgtgaatggtctacaatttgtcgtcaattatatgttcaagataaccgtgaattagttttagcagtttctatgacaaaaacaaaaaacgaagtttgtggttatccagttattgttgaattaatggataaaccattaacattccgtattttatctgatttaattttcgatttcgaagatgcaacaaaccaagcatctacaaaatctcataaatattctaaagttacaaactctttatctttaaaattaggtatttctttctctgaagatcaaaaaaaagaaaaagatcaattaggtaaagcaccaattgttttaccatctaacgttattaacttctgtcaagcagaaaaaccagaaccaacaaaccaagcagcaccattcaaagcaccagaattattaaaaggtcaatctgaagatgaacaaccagatgcagttgaagatgaacgtcaaatggatgatttacaaggtgcagcaccaccaattccaccacgtttagcacgtagtataatcaactttgaaaaactgagtcatcatcatcatcatcattaataa

SEQ ID NO: 654 - protein, 227 aa, artificial
  synthetic
PSRPSDAADEVRDTSINESRNVSNLEELPLNQYREYIKVQTDTTTLREWSTICRQLYVQDNRELVLAVSMTKTKNEVCGYPVIVELMDKPLTFRILSDLIFDFEDATNQASTKSHKYSKVTNSLSLKLGISFSEDQKKEKDQLGKAPIVLPSNVINFCQAEKPEPTNQAAPFKAPELLKGQSEDEQPDAVEDERQMDDLQGAAPPIPPRLARSIINFEKLSHHHHHH

SEQ ID NO: 655 - DNA, 372 bp, artificial
  synthetic
ttctcttctaacgttacaccaaaaatgttacaaattttcttatctgaaaaaaaatctatttctcaagcatgtacatctggtggttgtttacatattttaccaattacagttacaacagcacaagcaacatatcattctacatatttagttaaccaatctgcattcgatcaacgtgttgcaacagttcatttaggtaacggtacaattttatggattattgttttacaaccagcattacatcgtccaatgcattctcaatcttcttgttgtaaaccatgttgttgtcaatcttcttgttgtaaaccatgttgttgtgcacgtagtataatcaactttgaaaaactgagtcatcatcatcatcatcattaataa

SEQ ID NO: 656 - protein, 122 aa, artificial
  synthetic
FSSNVTPKMLQIFLSEKKSISQACTSGGCLHILPITVTTAQATYHSTYLVNQSAFDQRVATVHLGNGTILWIIVLQPALHRPMHSQSSCCKPCCCQSSCCKPCCCARSIINFEKLSHHHHHH

SEQ ID NO: 657 - DNA, 372 bp, artificial
  synthetic
acatctaactatacaggtttcgcaaacaaagatggttctaaaccatcttctttaggtgtttctttctgtggtggtgcattaattcatccatgtcgtgttttaacagcagcacattgtacagatattatggcattcgtttatttacgtgttttccgtaaagcacaaaaacaagttaaaaaaattgattctgcaggttgtcaattatggttatgtacattattagcagttttaaaagttggttggttattcccaaactatacaattttaatgcgtccacgtttcgttattttattaacagttatttcttggttcttagcacgtagtataatcaactttgaaaaactgagtcatcatcatcatcatcattaataa

SEQ ID NO: 658 - protein, 122 aa, artificial
  synthetic
TSNYTGFANKDGSKPSSLGVSFCGGALIHPCRVLTAAHCTDIMAFVYLRVFRKAQKQVKKIDSAGCQLWLCTLLAVLKVGWLFPNYTILMRPRFVILLTVISWFLARSIINFEKLSHHHHHH

SEQ ID NO: 659 - DNA, 372 bp, artificial
  synthetic
ccatctcgtccatctgatgcagcagatgaagttcgtgatacatctattaacgaatctcgtaacgtttctaacttagaagaattaccattaaaccaatatcgtgaatatattaaagttcaaacagatacaacaacattacgtgaatggtctacaatttgtcgtcaattatatgttcaagataaccgtgaattagttttagcagtttctatgacaaaaacaaaaaacgaagtttgtggttatccagttattgttgaattaatggataaaccattaacattccgtattttatctgatttaattttcgatttcgaagatgcacgtagtataatcaactttgaaaaactgagtcatcatcatcatcatcattaataa

SEQ ID NO: 660 - protein, 122 aa, artificial
  synthetic
PSRPSDAADEVRDTSINESRNVSNLEELPLNQYREYIKVQTDTTTLREWSTICRQLYVQDNRELVLAVSMTKTKNEVCGYPVIVELMDKPLTFRILSDLIFDFEDARSIINFEKLSHHHHHH

SEQ ID NO: 661 - DNA, 372 bp, artificial
  synthetic
gcaacaaaccaagcatctacaaaatctcataaatattctaaagttacaaactctttatctttaaaattaggtatttctttctctgaagatcaaaaaaaagaaaaagatcaattaggtaaagcaccaattgttttaccatctaacgttattaacttctgtcaagcagaaaaaccagaaccaacaaaccaagcagcaccattcaaagcaccagaattattaaaaggtcaatctgaagatgaacaaccagatgcagttgaagatgaacgtcaaatggatgatttacaaggtgcagcaccaccaattccaccacgtttagcacgtagtataatcaactttgaaaaactgagtcatcatcatcatcatcattaataa

SEQ ID NO: 662 - protein, 122 aa, artificial
  synthetic
ATNQASTKSHKYSKVTNSLSLKLGISFSEDQKKEKDQLGKAPIVLPSNVINFCQAEKPEPTNQAAPFKAPELLKGQSEDEQPDAVEDERQMDDLQGAAPPIPPRLARSIINFEKLSHHHHHH

SEQ ID NO: 663 - DNA, 687 bp, artificial
  synthetic
ttctcttctaacgttacaccaaaaatgttacaaattttcttatctgaaaaaaaatctatttctcaagcatgtacatctggtggttgtttacatattttaccaattacagttacaacagcacaagcaaaattaggtatttctttctctgaagatcaaaaaaaagaaaaagatcaattaggtaaagcaccagcaacaaaccaagcatctacaaaatctcataaatattctaaagttacaaactctttatctttaatggcattcgtttatttacgtgttttccgtaaagcacaaaaacaagttaaaaaaattgattctgcagcaccattcaaagcaccagaattattaaaaggtcaatctgaagatgaacaaccagatgcaacatatcattctacatatttagttaaccaatctgcattcgatcaacgtgttgcaacagttcatacatctaactatacaggtttcgcaaacaaagatggttctaaaccatcttctttaggtgtttctattgttttaccatctaacgttattaacttctgtcaagcagaaaaaccagaaccaacaaaccaagcaggttgtcaattatggttatgtacattattagcagttttaaaagttggttggttattcccagcacgtagtataatcaactttgaaaaactgagtcatcatcatcatcatcattaataa

SEQ ID NO: 664 - protein, 227 aa, artificial
  synthetic
FSSNVTPKMLQIFLSEKKSISQACTSGGCLHILPITVTTAQAKLGISFSEDQKKEKDQLGKAPATNQASTKSHKYSKVTNSLSLMAFVYLRVFRKAQKQVKKIDSAAPFKAPELLKGQSEDEQPDATYHSTYLVNQSAFDQRVATVHTSNYTGFANKDGSKPSSLGVSIVLPSNVINFCQAEKPEPTNQAGCQLWLCTLLAVLKVGWLFPARSIINFEKLSHHHHHH

SEQ ID NO: 665 - DNA, 372 bp, artificial
  synthetic
tctcaatcttcttgttgtaaaccatgttgttgtcaatcttcttgttgtaaaccatgttgttgtgaattaatggataaaccattaacattccgtattttatctgatttaattttcgatttcgaagatgtttctaacttagaagaattaccattaaaccaatatcgtgaatatattaaagttcaaacagatacaacaacattacgtgaatggtctacaatttgtcgtcaattatatgttcaagataaccgtgaattaggtaacggtacaattttatggattattgttttacaaccagcattacatcgtccaatgcatgcacgtagtataatcaactttgaaaaactgagtcatcatcatcatcatcattaataa

SEQ ID NO: 666 - protein, 122 aa, artificial
  synthetic
SQSSCCKPCCCQSSCCKPCCCELMDKPLTFRILSDLIFDFEDVSNLEELPLNQYREYIKVQTDTTTLREWSTICRQLYVQDNRELGNGTILWIIVLQPALHRPMHARSIINFEKLSHHHHHH

SEQ ID NO: 667 - DNA, 1317 bp, artificial
  synthetic
gaaacacataaagttgcagcaccagatgttattgttccaacattagatacagttcgtcatgaagttgcattagaattaaacggtggtttatgtgttgaagcatgtggtgcattagaaggtttatggttaacattaaacaacgatcaagttttcccaattaacccaccaaaattcgataaaattgaagattctattttaggtgaagaaccacgtttcggtaaaacaccattagcaatgttagcagcaacatgtacatatcattctacatatttagttaaccaatctgcattcgatcaacgtgttgcaacagttcatcgtgttgattatatttctacacgtcgtgcatctcaacataaatgtgcattattattacaagaaattcgtaacgcagttgaaatgatgattgcatggttagatacacaattaaaaaacaaattaattgataacatgccattagaattatctggtcgtttacaaaaaacagttacaggtttattaattgaatctcaatcttcttgttgtaaaccatgttgttgtcaatcttcttgttgtaaaccatgttgttgtttcatttgtcgtaaatgtcaaatgatgttcgcagatgaagatgcagcagttaaccatcaaaaatatccagaaattgttaaagaaaacaaattattagaacattattatcaagaattaaaaattgttgcattaaacacatctacatgtccaccaaaccgtgttaaattagatattttcccaaaagaatgtgcaaaaatgcgtcatggtttctctggtattttaattacagaaacaggtacaatgggttctaaattatgtgatttatctgatttagaatatttacatgaagaattccatcaatctttacaatggatgcgtttaacatatccaggtattaaagttgttgttgttattgatggtaactctgaagatgatttagcaattttattattattacaattatgggattgtggtgcacaagaaacagataacgaacgttctgttaaagttgcagaaattacagaattaattttcaaagcattagaaaacgatcaaaaagttcgtaaaggtgcatgttggatttatgataacattaacatggcacatatgttagttgcaatttctgttccaccaccaccaccattaccagcagcaccacaagcaccagaattcccatggatgaaagaaaaagaacgtatgttattccatggttctccattcttcaacgcaattattcataaaggtttcgatgaagcacgtagtataatcaactttgaaaaactgagtcatcatcatcatcatcattaataa

SEQ ID NO: 668 - protein, 437 aa, artificial
  synthetic
ETHKVAAPDVIVPTLDTVRHEVALELNGGLCVEACGALEGLWLTLNNDQVFPINPPKFDKIEDSILGEEPRFGKTPLAMLAATCTYHSTYLVNQSAFDQRVATVHRVDYISTRRASQHKCALLLQEIRNAVEMMIAWLDTQLKNKLIDNMPLELSGRLQKTVTGLLIESQSSCCKPCCCQSSCCKPCCCFICRKCQMMFADEDAAVNHQKYPEIVKENKLLEHYYQELKIVALNTSTCPPNRVKLDIFPKECAKMRHGFSGILITETGTMGSKLCDLSDLEYLHEEFHQSLQWMRLTYPGIKVVVVIDGNSEDDLAILLLLQLWDCGAQETDNERSVKVAEITELIFKALENDQKVRKGACWIYDNINMAHMLVAISVPPPPPLPAAPQAPEFPWMKEKERMLFHGSPFFNAIIHKGFDEARSIINFEKLSHHHHHH

SEQ ID NO: 669 - DNA, 1317 bp, artificial
  synthetic
ttaaacattccagcaccattacatccagaaaaaacagttaacatgacatatcaaccaacaacaggtttaccagaagcaaaaattttctctggtacatcttctgaacaattcggttatgcagttcaaccaccaccaccaccattaccagcagcaccacaagcaccagaattcccatggatgaaagaaaaaaaccaattaaaagaaatttcttcttctacagattctcaaaaaaaagaagaaacaaaaggtaaaggtgttgatcaacaagatggtaaaaaccattctggttctccaggttgtgaagaatctgatgcatctttacgtaacttatatacaccaaacgttatggtttctcgtttattaattttaggtggtgcagttcatgcacgtgcatgtggtcgtttcttccataaaggtcaaggtacatgtttctatttcggtggtatttctacacgtttaccaggtccaggtaaaccagttccaggtccagcaggtccagcacaagcaggtgaagcatctcgtttagcacattattctaaacgttctacaattacatctcgtgaaatttctttatctgttaaaattcgtgttatgccatgttctttcttcttattattacgtttcttcttagttgattctttcttacaagcagatggtccacaagaacgtccaccagcagcattaccagaatctggtttcaacgaaaaccgtcaaaaacgtgcagttttagcagcacaattcgaagcaacatctccagttaaagcagatccagcagcattaatggcattattacatttattaccatctccaacaccaaacttagaagaattatatgcattattcaaagcacaacatttaacatcttgttattggggtggttctacagttccaaaagaaccagcatctgcaccagaagcaccaacattaccaacaacagcaccagcaccaccaggtttattagttttattatggcaagttacaacattaccagaacattgtttctcttgggttcgtcgtacaaaattacaaatgcaaacagcagcattagaattccgtcaacgtattattttaccattaccaacaacagttggtgatttctggcatatgatttgggaacaaaaatctacagttattacattagttgattctcgtacatctgttacaaacgttaaattcgcaccaaaacatatgggtttagcaggttgtcaattatggttatgtacattattagcagttttaaaagttggttggttattcccagcacgtagtataatcaactttgaaaaactgagtcatcatcatcatcatcattaataa

SEQ ID NO: 670 - protein, 437 aa, artificial
  synthetic
LNIPAPLHPEKTVNMTYQPTTGLPEAKIFSGTSSEQFGYAVQPPPPPLPAAPQAPEFPWMKEKNQLKEISSSTDSQKKEETKGKGVDQQDGKNHSGSPGCEESDASLRNLYTPNVMVSRLLILGGAVHARACGRFFHKGQGTCFYFGGISTRLPGPGKPVPGPAGPAQAGEASRLAHYSKRSTITSREISLSVKIRVMPCSFFLLLRFFLVDSFLQADGPQERPPAALPESGFNENRQKRAVLAAQFEATSPVKADPAALMALLHLLPSPTPNLEELYALFKAQHLTSCYWGGSTVPKEPASAPEAPTLPTTAPAPPGLLVLLWQVTTLPEHCFSWVRRTKLQMQTAALEFRQRIILPLPTTVGDFWHMIWEQKSTVITLVDSRTSVTNVKFAPKHMGLAGCQLWLCTLLAVLKVGWLFPARSIINFEKLSHHHHHH

SEQ ID NO: 671 - DNA, 1317 bp, artificial
  synthetic
gcacgtagtataatcaactttgaaaaactgagtaacggtgttgtttctttagttggtggtccatgtacattaacatctgcaggtcaattattaccatctattttaggtgaagaaccacgtttcggtaaaacaccattagcaatgttagcagcaacatgtgatgaatatttaatttctcaatctgaacgttattctattacaatgacagaaaaatctacaattttatgtgatttatctgatttagaatatttacatgaagaattccatcaatctttacaatggatggaatctttccgtgcagcaattgataaatcttgtgatggtccagaagaaattgaagcagatggtacattaattcaaggtgttttacgtattaactctaaaaaattccatgaagcattcattccatctaaacaagcatctttagaattaggtgaaggtaaattaccagcagcatgtttaaaaccagcattagattctgaaggtccagcagaaggtgaaggtcatttaggtcgttgtatgcgtcgtttacgtgatgcagttcgtcgtttacgttataaaggtggtgatacattcacaggtttagcattaacacatgttgttcgtacatgtatgcataaccaaaaagatgttgttcaaatgccatctgaaacattaaaagcagataacatgccattagaattatctggtcgtttacaaaaaacagttacaggtttattaattgaacaatcttcttgtcaaccagcatgttgtacaacatcttcttgtcaacaagcatgttgtgttccaattaaagttaaatctgaagaaaaaattaaagcagttgaaaaatctgaaaaagaaacagttattgttcgtcgtacaaaattacaaatgcaaacagcagcattagaattccgtcaacgtattattttagatttatatatggatacaggtgaagaaatttgtttccgtgttgttgatgaatctttcgttgatgcattaaacacatctacatgtccaccaaaccgtgttaaattagatattttcccaaaagaatgtgaatatgttaaatggttcttacatgcagatttatcttcttatttaacaccaatgaaacatatgatttctcgtgatgcagcacaaaaaatttatttagaaatgaaattcacattacgtgattgtaacaactgttctttcgaagatttcacagaatctaaatctgttttagaagatggtcgtattcattgttctaaaaaattcttagaaaacttattagaaattttcaactctcatgttgatcatggtacaggtcatcatcatcatcatcattaataa

SEQ ID NO: 672 - protein, 437 aa, artificial
  synthetic
ARSIINFEKLSNGVVSLVGGPCTLTSAGQLLPSILGEEPRFGKTPLAMLAATCDEYLISQSERYSITMTEKSTILCDLSDLEYLHEEFHQSLQWMESFRAAIDKSCDGPEEIEADGTLIQGVLRINSKKFHEAFIPSKQASLELGEGKLPAACLKPALDSEGPAEGEGHLGRCMRRLRDAVRRLRYKGGDTFTGLALTHVVRTCMHNQKDVVQMPSETLKADNMPLELSGRLQKTVTGLLIEQSSCQPACCTTSSCQQACCVPIKVKSEEKIKAVEKSEKETVIVRRTKLQMQTAALEFRQRIILDLYMDTGEEICFRVVDESFVDALNTSTCPPNRVKLDIFPKECEYVKWFLHADLSSYLTPMKHMISRDAAQKIYLEMKFTLRDCNNCSFEDFTESKSVLEDGRIHCSKKFLENLLEIFNSHVDHGTGHHHHHH

SEQ ID NO: 673 - DNA, 372 bp, artificial
  synthetic
ccaccattaccagatgtttctgcattctcttctggtcgtggtggtggtgaaggtcgtggttctaaccaaccacgtttaattcaatgtgaaaacaaagttggtaaattattattcattacagaaattaaatctacaacaaaacgttctccaaaaccacaaaacaaaaaaaaaacaaaaaaagttattgaaaaacgtaaagattctcgtacagtttatcaatctcaatatacaaactggtctgttgaacaattaaacttacaattcgaatatattgatgatccatgggttcaacgtattgaaccagaatggtctattgcacgtagtataatcaactttgaaaaactgagtcatcatcatcatcatcattaataa

SEQ ID NO: 674 - protein, 122 aa, artificial
  synthetic
PPLPDVSAFSSGRGGGEGRGSNQPRLIQCENKVGKLLFITEIKSTTKRSPKPQNKKKTKKVIEKRKDSRTVYQSQYTNWSVEQLNLQFEYIDDPWVQRIEPEWSIARSIINFEKLSHHHHHH

SEQ ID NO: 675 - DNA, 1317 bp, artificial
  synthetic
ggtgaagttttaccaacaccaggtcaagaaccaccaacaccaattcatgatttatctgcatctaaacgtaaagattctcgtacagtttatcaatctcaatatacaaactggtctgttgaacaattaccaccattaccagatgtttctgcattctcttctggtcgtggtggtggtgaaggtcgtggttctaaccaaccacgtttaattcaatgtgaaaacaaagttggtaaattattattcattacagaaatttctaaacaacaagttgcagatgaagatgaaatgtggaaacaacgtcgtcgtcaacaatctgaaattacagttcatacaccattattaccatctgcaacaggtttattaaacgataacacattcgcagcatggcaaaactctattcgtcataacttatgtttaaacgattgtttcgttaaaattccacgtaaatctacaacaaaacgttctccaaaaccacaaaacaaaaaaaaaacaaaaaaagttattgaatctttatctgttaaaattcgtgttatgccatgttctttcttcttattattacgtttcttcttaacaacatctaaagattggcatcatggtggtccagaaaacattttattatgtacagattgtcgtccatctgttccattaggtggtgcaccatcttgtggtatgttaatggataaaccacatccaccagaacgtacaacagaagaagttgcacgtaaccaacgtgcagttgaagcagaaaaatgtgcacgtgcaggtgaagcatctcgtttagcacattattctaaacgttctacaattacatctcgtgaaattgatggtttaccaggtccaccaggtacaatgatgatgttaccattccgttatggtggtgatggtaacttacaattcgaatatattgatgatccatgggttcaacgtattgaaccagaatggtctattgaaatgaacaaacaaatttctttattaacattcgcagttaaccatttaaaagcaaacgttaaacgttataacccatctttaaaaggtttaaacgttacaggttatgaaacacaagcaggtgaattcgttcaattacgtgatggtgattctaactgtcaatggtctgttttatatgttattattgcaacagtttctcgtgcagcagttcattgtgcattatcttctccaccaccagaagcacgtgcattagcatctttcccagcacaagatccatgtacaacaacatatgttgcagcaacagaaccagttccagaagcacgtagtataatcaactttgaaaaactgagtcatcatcatcatcatcattaataa

SEQ ID NO: 676 - protein, 437 aa, artificial
  synthetic
GEVLPTPGQEPPTPIHDLSASKRKDSRTVYQSQYTNWSVEQLPPLPDVSAFSSGRGGGEGRGSNQPRLIQCENKVGKLLFITEISKQQVADEDEMWKQRRRQQSEITVHTPLLPSATGLLNDNTFAAWQNSIRHNLCLNDCFVKIPRKSTTKRSPKPQNKKKTKKVIESLSVKIRVMPCSFFLLLRFFLTTSKDWHHGGPENILLCTDCRPSVPLGGAPSCGMLMDKPHPPERTTEEVARNQRAVEAEKCARAGEASRLAHYSKRSTITSREIDGLPGPPGTMMMLPFRYGGDGNLQFEYIDDPWVQRIEPEWSIEMNKQISLLTFAVNHLKANVKRYNPSLKGLNVTGYETQAGEFVQLRDGDSNCQWSVLYVIIATVSRAAVHCALSSPPPEARALASFPAQDPCTTTYVAATEPVPEARSIINFEKLSHHHHHH

SEQ ID NO: 677 - DNA, 687 bp, artificial
  synthetic
ggtgaagttttaccaacaccaggtcaagaaccaccaacaccaattcatgatttatctgcatctaaacgtaaagattctcgtacagtttatcaatctcaatatacaaactggtctgttgaacaattaccaccattaccagatgtttctgcattctcttctggtcgtggtggtggtgaaggtcgtggttctaaccaaccacgtttaattcaatgtgaaaacaaagttggtaaattattattcattacagaaatttctaaacaacaagttgcagatgaagatgaaatgtggaaacaacgtcgtcgtcaacaatctgaaattacagttcatacaccattattaccatctgcaacaggtttattaaacgataacacattcgcagcatggcaaaactctattcgtcataacttatgtttaaacgattgtttcgttaaaattccacgtaaatctacaacaaaacgttctccaaaaccacaaaacaaaaaaaaaacaaaaaaagttattgaatctttatctgttaaaattcgtgttatgccatgttctttcttcttattattacgtttcttcttaacaacatctaaagattggcatcatggtggtccagaaaacattttattatgtacagattgtcgtgcacgtagtataatcaactttgaaaaactgagtcatcatcatcatcatcattaataa

SEQ ID NO: 678 - protein, 227 aa, artificial
  synthetic
GEVLPTPGQEPPTPIHDLSASKRKDSRTVYQSQYTNWSVEQLPPLPDVSAFSSGRGGGEGRGSNQPRLIQCENKVGKLLFITEISKQQVADEDEMWKQRRRQQSEITVHTPLLPSATGLLNDNTFAAWQNSIRHNLCLNDCFVKIPRKSTTKRSPKPQNKKKTKKVIESLSVKIRVMPCSFFLLLRFFLTTSKDWHHGGPENILLCTDCRARSIINFEKLSHHHHHH

SEQ ID NO: 679 - DNA, 945 bp, artificial
  synthetic
ccatctttccaagaattcgttgattgggaaaacgtttctccagaattaaactctacagatcaaggtggtggaggaaaagcatatttaccagttaacgaatctttcgcattcacagcagatttacgttctaacacaggtggtggaggtggacgtaacccacaattcttagatccagttttagcatatttaatgaaaggtttatgtgaaaaaccaggtggaggaggtaaagcattcttacattggtatacaggtgaagcaatggatgaaatggaattcacagaagcagaaggaggtggtggagatttctctcaattacaacgtaacattttaccatctaacccacgtgttacacgtttccatattggaggaggtggtattacaccaccaacaacaacaacaaaaaaagcacgtgtttctacaccaaaaccagcaacaccaggaggtggaggtaactataacacatctcatttaaacaacgatgtttggcaaattttcgaaaacccagttgattggggaggaggaggtcatattaaagcattcgatcgtacattcgcaaacaacccaggtccaatggttgttttcgcaacaggaggaggtggaattacatctaacttcgttattccatctgaatattgggttgaagaaaaagaagaaaaacaaaaaggaggtggaggaggtttagttacattccaagcattcattgatgttatgtctcgtgaaacaacagatacagatacaggtggaggaggacattggaacgatttagcagttattccagcaggtgttgttcataactgggatttcgaaccacgtggaggaggaggacaaccattacgtcgtttagttttacatgttgtttctgcagcacaagcagaacgtttagcacgtgcacgttctattattaacttcgaaaaattatctcatcatcatcatcatcattaataa

SEQ ID NO: 680 - DNA, 1485 bp, artificial
  synthetic
ccatctttccaagaattcgttgattgggaaaacgtttctccagaattaaactctacagatcaaggtggtggaggattcatggttgcagttgcacatgttgcagcattcttattagaagatcgtgcagtttgtgttggaggaggaggagcagaaaacgttgaacaagttttagttacatctattcaaggtgcagttgattatccagatccaggtggaggtggatctttcaaaaaaaaattcgaagaatgtcaacataacattattaaattacaaaacggtcatacaggtggaggaggttctgcattaattgaatctttaaaccaaaaaacacaatctacaggtgatcatccacaaccaacaggaggtggtggaaaagcatatttaccagttaacgaatctttcgcattcacagcagatttacgttctaacacaggtggaggaggtggtcatacattattagaaattacagaagaatctggtgcagttttagttgataaatctgattctgatggaggtggaggttctgttatgtgtacatattctccaccattagataaattattctgtcaattagcaaaaacatgtggaggaggaggtgaatctggtaaacataaatatcgtcaaacagcaatgttcacagcaacaatgccaccagcagttggaggaggtggagcagcaccatctgcagcatcttctccagcagatgttcaatctttaaaaaaagcaatgtcttctggaggtggaggatctcaattattctctttaaacccacgtggtcgttctttagttacagcaggtcgtattgatcgtggtggaggaggatctttagcacgtggtccattatctgaagcaggtttagcattattcgatccatattctaaagaaggaggaggaggacaaaaaaaattatgtcatttatcttctacaggtttaccacgtgaaacaattgcatctttaccaggtggtggaggattaacagcatctaacatggaaggtaaatcttggccatctgaagttttagtttgtacaacatctggtggaggtggatatgcagcacaacaacatgaaacattcttaacaaacggtgatcgtgcaggtttcttaattggtggtggaggaggtcaagcaaaagttccattctctgaagaaacacaaaacttaattttaccatatatttctgatatgggaggtggtggatgtaaccgtgcaggtgaaaaacattgtttctcttctaacgaagcagcacgtgatttcggtggtggaggaggtggtcgtaacccacaattcttagatccagttttagcatatttaatgaaaggtttatgtgaaaaaccaggaggtggaggtttagaatgtgaacgtggtaaacaagaagcaaaattattagcagaacgttctcgtttcgaagatggaggaggaggtgcaccattagaatggttacgttatttcgataaaaaagaattagaattaatgttatgtggtatg

SEQ ID NO: 681 - DNA, 1884 bp, artificial
  synthetic
ccatctttccaagaattcgttgattgggaaaacgtttctccagaattaaactctacagatcaagcagaaaacgttgaacaagttttagttacatctattcaaggtgcagttgattatccagatccatctttcaaaaaaaaattcgaagaatgtcaacataacattattaaattacaaaacggtcatacatctgcattaattgaatctttaaaccaaaaaacacaatctacaggtgatcatccacaaccaacaaaagcatatttaccagttaacgaatctttcgcattcacagcagatttacgttctaacacaggtcatacattattagaaattacagaagaatctggtgcagttttagttgataaatctgattctgattctgttatgtgtacatattctccaccattagataaattattctgtcaattagcaaaaacatgtgaatctggtaaacataaatatcgtcaaacagcaatgttcacagcaacaatgccaccagcagttgcagcaccatctgcagcatcttctccagcagatgttcaatctttaaaaaaagcaatgtcttcttctcaattattctctttaaacccacgtggtcgttctttagttacagcaggtcgtattgatcgttctttagcacgtggtccattatctgaagcaggtttagcattattcgatccatattctaaagaacaaaaaaaattatgtcatttatcttctacaggtttaccacgtgaaacaattgcatctttaccattaacagcatctaacatggaaggtaaatcttggccatctgaagttttagtttgtacaacatcttatgcagcacaacaacatgaaacattcttaacaaacggtgatcgtgcaggtttcttaattggtcaagcaaaagttccattctctgaagaaacacaaaacttaattttaccatatatttctgatatgtgtaaccgtgcaggtgaaaaacattgtttctcttctaacgaagcagcacgtgatttcggtggtcgtaacccacaattcttagatccagttttagcatatttaatgaaaggtttatgtgaaaaaccattagaatgtgaacgtggtaaacaagaagcaaaattattagcagaacgttctcgtttcgaagatgcaccattagaatggttacgttatttcgataaaaaagaattagaattaatgttatgtggtatgccagcattagttgaagaatatttagaacgtggtaacttcgttgcaaacgatttagattggttagaattaaaagcatgtaaaccaaacggtaaacgtaacccatattgtgaagtttctatgggttcttctccattcccagcagcagttattttacgtgatgcattacatatggcacgtggtttaaaatatcaacaattagatacatatattttaaaaaacgttgttgcattctctcgtacagataaatatcgttctttcgttggtcaaacacgtgttttaatgattaacggtgaagaagttgaagaaacagaattagcattcttcattaacttcattgcaatttatcatcatgcatctcgtgcaattccattcggtacaggtttagcattaccaaacaactattgtgatgtttgtttaggtgattctaaaattaacaaaaaagaaggtcaaatttctattgcaaaatatgaaaactgtccaaaagataacccaatgtattattgtaacttcaaacgtaaacgtgttgcagcattccaaaaaaacttaattgaaatgtctgaattagaaaaaatgaaaggtgaattaggtatgatgttaattttacaaaacgttattcaaaaaacaacaacagcacgttctattattaacttcgaaaaattatctcatcatcatcatcatcattaataa

SEQ ID NO: 682 - protein, 179 aa, artificial
  synthetic
FRRKAFLHWYTGEAMDEMEFTEAESNMPKPDFSQLQRNILPSNPRVTRFHINWDTAVITPPTTTTKKARVSTPKPATPSTDNHSGLVTFQAFIDVMSRETTDTDTADQSHCHWNDLAVIPAGVVHNWDFEPRKVSGFSQPLRRLVLHVVSAAQAERLARAEEARSIINFEKLSHHHHHH

SEQ ID NO: 683 - protein, 233 aa, artificial
  synthetic
FRRKAFLHWYTGEAMDEMEFTEAESNMPKPDFSQLQRNILPSNPRVTRFHINWDTAVITPPTTTTKKARVSTPKPATPSTDNHSGLVTFQAFIDVMSRETTDTDTADQSHCHWNDLAVIPAGVVHNWDFEPRKVSGFSQPLRRLVLHVVSAAQAERLARAEEPSKPSFQEFVDWENVSPELNSTDQPFLEFKHIKAFDRTFANNPGPMVVFATPGMARSIINFEKLSHHHHHH

SEQ ID NO: 684 - protein, 368 aa, artificial
  synthetic
SGCYFMVAVAHVAAFLLEDRAVCVERFMTESFKKKFEECQHNIIKLQNGHTSLAPRVSALIESLNQKTQSTGDHPQPTDTTGSSPDVHTLLEITEESGAVLVDKSDSDTAKSVMCTYSPPLDKLFCQLAKTCPVQQRAAAPSAASSPADVQSLKKAMSSLQNRGQSQLFSLNPRGRSLVTAGRIDREELASSSLARGPLSEAGLALFDPYSKEESTKFRLTASNMEGKSWPSEVLVCTTSPDRAVTYAAQQHETFLTNGDRAGFLIGDGAWLPQAKVPFSEETQNLILPYISDMNFVVETLECERGKQEAKLLAERSRFEDEKQNEVAPLEWLRYFDKKELELMLCGMQEIARSIINFEKLSHHHHHH

SEQ ID NO: 685 - protein, 395 aa, artificial
  synthetic
LILISTNGSFIRLLDAFKGVVMHTFGGREGVELCPGNKYEMRRHGTTHSLVIHDDNIPALVEEYLERGNFVANDLDWLLALDSGSPFPAAVILRDALHMARGLKYLHQHLTQQLDTYILKNVVAFSRTDKYRQLPLVLSFVGQTRVLMINGEEVEETELMGFGPDGLALPNNYCDVCLGDSKINKKTGQSCTEGQISIAKYENCPKDNPMYYCNKKELINFKRKRVAAFQKNLIEMSELEIKHNREKMKGELGMMLILQNVIQKTTTPGEHSLSIECKGIDKEINESKNTHLDIPRITLTELEAAIETVVCTFFTFAGREGRKGGRGHLLGRLAAIVGKQVLLGRKVVVVREKFSMDHKTGTIAMQNTTQLRSRYELTARSIINFEKLSHHHHHH

SEQ ID NO: 686 - protein, 44 aa, artificial
  synthetic
PSKPSFQEFVDWENVSPELNSTDQPFLARSIINFEKLSHHHHHH

SEQ ID NO: 687 - protein, 44 aa, artificial
  synthetic
FRRKAFLHWYTGEAMDEMEFTEAESNMARSIINFEKLSHHHHHH

SEQ ID NO: 688 - protein, 44 aa, artificial
  synthetic
GFSQPLRRLVLHVVSAAQAERLARAEEARSIINFEKLSHHHHHH